(19) United States
(12) Patent Application Publication
(10) Pub. No.: US 2024/0287138 A1
(43) Pub. Date: Aug. 29, 2024

(54) CYCLIC PEPTIDES FOR INHIBITING TNF RECEPTOR 1 ACTIVITY

(71) Applicant: Merck Sharp & Dohme LLC, Rahway, NJ (US)

(72) Inventors: Kaustav Biswas, Newton, MA (US); Nicolas C. Boyer, Somerville, MA (US); Brandon D. Cash, Natick, MA (US); Fa-Xiang Ding, Staten Island, NY (US); John R. Frost, Summit, NJ (US); Michael B. Garrigou, Newton, MA (US); James P. Jewell, Newton, MA (US); Ahmet Kekec, Hoboken, NJ (US); Hu-Jung Julie Lee, Cranford, NJ (US); Songnian Lin, Holmdel, NJ (US); Ashwin U. Rao, Morganville, NJ (US); Daniel J. Sindhikara, East Brunswick, NJ (US); Murray Wan, Medford, MA (US); Yuping Zhu, Basking Ridge, NJ (US); Susan L. Zultanski, Brooklyn, NY (US); Tony Siu, Wellesley, MA (US); Andrew M. Haidle, Somerville, MA (US); Claudio Mapelli, Yardley, PA (US); Luca Gambini, Rome (IT); Federica Orvieto, Rome (IT); Roberta Tozzi, Rome (IT); Nicolo Maria Pasquini, Rome (IT); Luigi Abate, Rome (IT); Giordano Proietti, Rome (IT); Michael Wuo, Cambridge, MA (US); Ryan Chau, Boston, MA (US)

(21) Appl. No.: 18/429,587

(22) Filed: Feb. 1, 2024

Related U.S. Application Data
(60) Provisional application No. 63/483,140, filed on Feb. 3, 2023.

(51) Int. Cl. C07K 7/54; A61P 29/00

(52) U.S. Cl. CPC C07K 7/54; A61P 29/00

(57) ABSTRACT

Provided are compounds of the Formula (I), or their pharmaceutically acceptable salts,

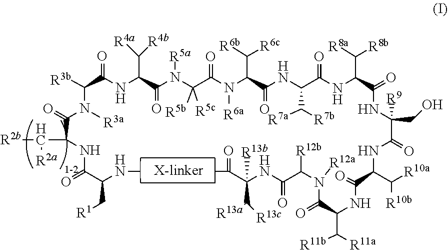

can inhibit TNFR1 and are expected to have utility as therapeutic agents, for example, for treating inflammatory bowel diseases, rheumatoid arthritis, junvenile rheumatoid arthritis, psoriasis, psoriatic arthritis, ankylosing spondylitis, non-radiographic axial spondyloarthritis and hidradenitis suppurativa. The disclosure also provides pharmaceutical compositions which comprise the compounds disclosed herein or pharmaceutically acceptable salts thereof. The disclosure also relates to methods for use of the compounds or their pharmaceutically acceptable salts in the therapy and prophylaxis of inflammatory bowel diseases, rheumatoid arthritis, juvenile rheumatoid arthritis, psoriasis, psoriatic arthritis, ankylosing spondylitis, non-radiographic axial spondyloarthritis and hidradenitis suppurativa, and for preparing pharmaceuticals for this purpose.

### CROSS-REFERENCE TO RELATED APPLICATIONS

This application claims the benefit of U.S. Provisional Patent Application Ser. No. 63/483,140 filed Feb. 3, 2023, the entire contents of which is incorporated by reference herein.

### REFERENCE TO SEQUENCE LISTING SUBMITTED ELECTRONICALLY

The contents of the electronic sequence listing (25644-WO-PCT_SL.xml; Size: 1,347,000 bytes; and Date of Creation: Jan. 19, 2024) are herein incorporated by reference in their entirety.

### FIELD OF THE INVENTION

The present disclosure relates to certain cyclic peptides that inhibit TNF receptor 1 (TNFR1) activity, pharmaceutical compositions comprising such peptides, and methods for using the compounds for treating, inhibiting, or ameliorating one or more autoimmune and inflammatory disease states that could benefit from inhibiting TNFR1, including inflammatory bowel diseases (IBD), rheumatoid arthritis (RA), juvenile rheumatoid arthritis (JRA), psoriasis, psoriatic arthritis, ankylosing spondylitis, non-radiographic axial spondyloarthritis, hidradenitis suppurativa and other dermatological disorders, as well as other neurological, neurodegenerative, metabolic and ocular disorders.

### BACKGROUND OF THE INVENTION

Therapeutics which target the signaling of the cytokine tumor necrosis factor alpha (TNFα), comprising the anti-TNFα biologics which emerged in the 1990s, continue to be among the standard of care (SOC) for several prevalent autoimmune and inflammatory diseases including ulcerative colitis (UC), Crohn's disease (CD), collectively referred to as IBD, RA, juvenile RA, psoriasis, psoriatic arthritis, ankylosing spondylitis, non-radiographic axial spondyloarthritis, and hidradenitis suppurativa. TNFα is a pleiotropic cytokine that affects the function of a variety of cell types. It triggers cellular responses from the induction of inflammatory gene expression programs, the stimulation of cellular proliferation and differentiation, to the activation of cellular suicide programs such as apoptosis and necroptosis. It is expressed as a type II single spanning transmembrane protein and as a soluble variant released after proteolytic processing.

TNFα self-assembles into homo-trimeric molecules and both the transmembrane and soluble form interact with the two known membrane receptors of TNFα, TNFR1 and TNFR2, each exerting distinct biological effects. Both receptors of TNFα are typical representatives of the broader TNF receptor superfamily. As such, TNFR1 and TNFR2 are single-spanning type I transmembrane proteins characterized by having several cysteine-rich domains in their extracellular domains. Soluble forms of TNFR1 and TNFR2 have also been described and result from alternative splicing or shedding. TNFR1 is expressed by almost all cell types and mediates the well-known pro-inflammatory, cytotoxic, and gene inductive signaling actions of TNFα. TNFR2 is expressed by more restricted cell types, including myeloid cells, regulatory T-cells, glial cells, some endothelial cell types, epithelial cells, fibroblasts, and certain T- and B-cell subsets and induces immunosuppressive/homeostatic effects of the cytokine on immune cells and in tissue regeneration.

The mechanism of action (MOA) of anti-TNFα biologics such as infliximab, adalimumab, golimumab and certolizumab, involves binding to the cytokine TNFα and thus, inhibition of engagement with both TNFR1 and TNFR2.

A key limitation of anti-TNFα SOC biologics as a medicine class is the high rate of loss of response (upwards of >70% within one year of treatment) which in many cases is caused by the development of anti-drug antibodies (ADA, ˜60% among IBD patients) which lead to undesirable changes in the pharmacokinetic and pharmacodynamic properties of these drugs (See, Colombel et al., “Adalimumab for Maintenance of Clinical Response and Remission in Patients with Crohn's Disease”, Gastroenterology 132 (1): 52-65 (2007); and Vaisman-Mentesh, A. et al., “The Molecular Mechanisms that underlie the Immune Biology of Anti-drug Antibody Formation following Treatment with Monoclonal Antibodies”, Frontiers in Immunology, 11:1951 (2020)).

The limitations of anti-TNF therapy may depend on TNF's pleiotropic biological functions via two distinct TNF receptors. In different animal disease models, genetic deletion of TNFR1 is typically associated with or reduced disease, whereas TNFR2 ablation exacerbates disease. These and other data indicate that soluble TNFα/TNFR1 signaling mainly mediates pro-apoptotic and inflammatory responses, whereas TNFR2 contributes to immune regulation and tissue regeneration. Therefore, reagents that selectively target TNFRs might be superior to global TNF blockade because they allow a differential activation and/or inhibition of TNFRs. Selective blocking of TNFα/TNFR1 signaling, which will preserve functional TNFα/TNFR2 signaling, seems to be sufficient to interfere with pathological TNFα signaling. In contrast to global TNF blockers that neutralize soluble and transmembrane TNFα, this class of therapeutics may induce less severe side-effects and may be therapeutic for other diseases such as MS or neurodegenerative diseases, where complete TNF inhibition is contraindicative (See, Fischer et al., “Selective Targeting of TNF Receptors as a Novel Therapeutic Approach”, Front. Cell Dev. Biol. 8:401(2020); Dong et al., “Targeting of Tumor Necrosis Factor Alpha Receptors as a Therapeutic Strategy for Neurodegenerative Disorders”, Antibodies, 4:4 (2015))

There is a need for additional, therapeutic approaches beyond the standard of care of current anti-TNFα biologics medications for slowing the progression of prevalent autoimmune and inflammatory diseases.

### SUMMARY OF THE DISCLOSURE

The present disclosure provides certain cyclic peptides that reduce inflammation by selectively inhibiting TNFR1 to specifically attenuate the proinflammatory activities of TNFα mediated by TNFR1 signaling, and to spare/passively enable TNFα-TNFR2 pro-homeostatic signaling. These cyclic peptides can be valuable pharmaceutically active compounds for the treatment of prevalent autoimmune and inflammatory diseases including ulcerative colitis (UC), Crohn's disease (CD), collectively referred to as inflammatory bowel diseases (IBD), as well as rheumatoid arthritis (RA), juvenile rheumatoid arthritis (JRA), psoriasis and other inflammatory conditions that can be treated by blockade of TNFα signaling like psoriatic arthritis, ankylosing spondylitis, non-radiographic axial spondyloarthritis, hidradenitis suppurativa, and other dermatological disorders, as well as other neurological, neurodegenerative, metabolic and ocular disorders.

In one aspect, the present disclosure provides compounds of Formula (I)

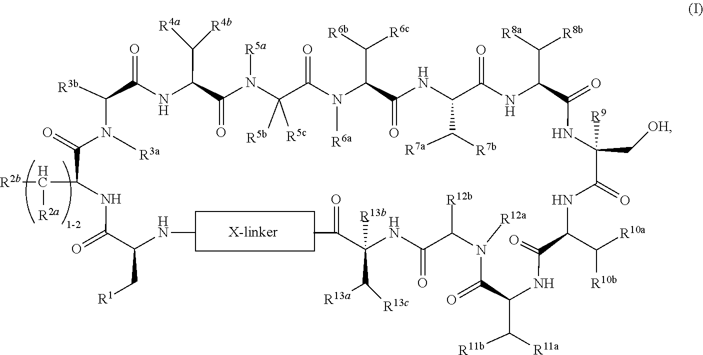

and their pharmaceutically acceptable salts.

The compounds of the present invention have the potential to provide superior, more durable efficacy over non-selective anti-TNFα SOC, through inhibition of TNFR1-driven inflammation that concomitantly spares TNFR2-dependent homeostasis, mucosal/tissue healing, and immune regulation. Accordingly, in another aspect, the present disclosure provides a method for treating autoimmune and inflammatory diseases (e.g., ulcerative colitis, Crohn's disease, collectively referred to as IBD, rheumatoid arthritis, juvenile rheumatoid arthritis, psoriasis, psoriatic arthritis, ankylosing spondylitis, non-radiographic axial spondyloarthritis and hidradenitis suppurativa) comprising administering a therapeutically effective amount of the compound of the disclosure to a subject in need thereof. In some embodiments, the administration comprises an oral administration of the compound.

The disclosure furthermore provides processes for preparing compounds of the disclosure and pharmaceutical compositions which comprise compounds of the disclosure and a pharmaceutically acceptable carrier.

### DETAILED DESCRIPTION OF THE INVENTION

### Compounds of the Disclosure

In one embodiment, the present disclosure provides a compound having structural Formula (I) or a pharmaceutically acceptable salt thereof:

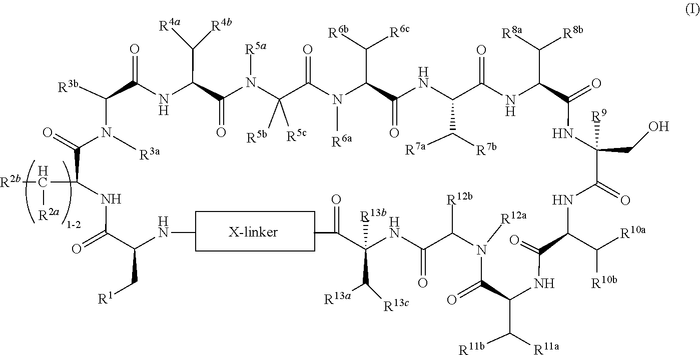

R¹ is selected from hydrogen, C₁₋₁₀ alkyl, (C₁₋₆ alkyl)₀₋₂ amino(C₀₋₁₀ alkyl), (C₁₋₆ alkyl)₀₋₂ amino(C₀₋₁₀ alkyl)oxy(C₀₋₆ alkyl), (C₁₋₆ alkyl)₃N⁺(C₀₋₆ alkyl), aryl(C₀₋₁₀ alkyl), heteroaryl(C₀₋₁₀ alkyl), (C₃₋₁₂)cycloalkyl(C₀₋₁₀ alkyl), heterocycloalkyl(C₀₋₁₀ alkyl), C₁₋₁₀ fluoroalkyl, C₂₋₁₀ alkenyl, (C₀₋₆ alkyl)carbonylamino(C₀₋₆ alkyl), (C₀₋₆ alkyl)-2 aminocarbonyl(C₀₋₆ alkyl), (C₁₋₆ alkyl)₀₋₂ aminocarbonylamino(C₀₋₆ alkyl), arylcarbonylamino(C₀₋₆ alkyl), arylaminocarbonyl(C₀₋₆ alkyl), heteroarylcarbonylamino(C₀₋₆ alkyl), heteroarylaminocarbonyl(C₀₋₆ alkyl), C₁₋₆ alkyloxy, (C₁₋₆ alkyl)oxy(C₀₋₆ alkyl), ((C₃₋₁₂)cycloalkyl)oxy(C₀₋₆ alkyl), ((C₃₋₁₂)cycloalkyl C₀₋₆ alkyl)oxy(C₀₋₆ alkyl), (C₀₋₆ alkyl)carboxy(C₀₋₆ alkyl), N⁻═N⁺═N—(C₀₋₆ alkyl), and H₂N—C(═NH)NH—(C₀₋₆ alkyl),
wherein R¹ is substituted by 0, 1, 2, 3, or 4 R^{1a} substituents each independently selected from C₁₋₆ alkyl, amino, cyano, halo, hydroxy, (C₃₋₁₂)cycloalkyloxy, and C₁₋₆ alkyloxy;
each R^{2a} is independently selected from hydrogen, hydroxy, C₁₋₄ alkyl, fluoro, and C₁₋₄ alkyloxy;
R^{2b} is selected from aryl, heteroaryl, cycloalkyl, and heterocycloalkyl, wherein said heteroaryl contains at least one nitrogen atom,
wherein R^{2b} is substituted by 0, 1, or 2 R^{2c} independently selected from C₁₋₆ alkyl, amino(C₀₋₆ alkyl), (C₁₋₆ alkyl)₀₋₂ amino(C₀₋₆ alkyl), (C₁₋₆ alkyl)₃N⁺(C₀₋₆ alkyl), aminocarbonyl(C₀₋₆ alkyl), (C₁₋₆ alkyl)₀₋₂ aminocarbonyl(C₀₋₆ alkyl), hydroxy, C₁₋₆ alkyloxy, halo, (C₁₋₆ alkyl)₀₋₂ amino(C₀₋₆ alkyloxy), (C₁₋₆ alkyl)₃N⁺(C₀₋₆ alkyloxy), (C₁₋₆ alkyloxy)carbonyl(C₀₋₆ alkyl), carboxy(C₀₋₆ alkyl), carboxy(C₁₋₆ alkyl)oxy(C₀₋₆ alkyl), C₁₋₆ haloalkyl, C₁₋₆ haloalkyloxy, and C₁₋₆ alkyloxy;
R^{3a} is selected from hydrogen, hydroxy, hydroxy(C₁₋₆ alkyl), amino, amino(C₁₋₆ alkyl), C₁₋₁₀ alkyl, (C₃₋₁₂)cycloalkyl(C₀₋₁₀ alkyl), (C₀₋₆ alkyl)thio(C₁₋₆ alkyl), and carboxy(C₁₋₆ alkyl), wherein R^{3a} may be substituted by 0, 1, or 2 R^{3c} substituents;
R^{3b} is selected from hydrogen, C₁₋₁₀ alkyl, hydroxy(C₁₋₆ alkyl), amino(C₁₋₁₀ alkyl), (C₁₋₆ alkyl)₀₋₂ amino(C₁₋₁₀ alkyl), (C₁₋₆ alkyl)₃N⁺(C₁₋₆ alkyl), C₁₋₆ haloalkyl, aryl(C₀₋₁₀ alkyl), heteroaryl(C₀₋₁₀ alkyl), (C₃₋₁₂)cycloalkyl(C₀₋₁₀ alkyl), heterocycloalkyl(C₀₋₁₀ alkyl), (C₁₋₆ alkyl)oxy(C₁₋₆ alkyl), (C₃₋₁₂)cycloalkyloxy(C₁₋₆ alkyl), carboxy(C₁₋₆ alkyl), aminocarbonyl(C₁₋₆ alkyl), (C₁₋₆ alkyl)₀₋₂ aminocarbonyl(C₁₋₆ alkyl), aminocarbonylamino(C₁₋₆ alkyl), (C₁₋₆ alkyl)₀₋₂ aminocarbonylamino(C₁₋₆ alkyl), (C₀₋₆ alkyl)thio(C₁₋₆ alkyl), (C₁₋₆ alkyl)SO₂(C₁₋₆ alkyl), and (C₁₋₆ alkyl)sulfinyl(C₁₋₆ alkyl), wherein R^{3b} may be substituted by 0, 1, or 2 R^{3c} substituents;
each R^{3c} is independently selected from halo, C₁₋₆ alkyl, amino, (C₁₋₆ alkyl)₀₋₂ amino(C₀₋₆ alkyl), (C₁₋₆ alkyl)₃N⁺—, (C₁₋₆ alkyl)SO₂(C₀₋₆ alkyl), cyano, cyano(C₁₋₆ alkyl), hydroxy, hydroxy(C₁₋₆ alkyl), (C₁₋₆ alkyl)oxy(C₀₋₆ alkyl), aminocarbonyl(C₀₋₆ alkyl), and (C₀₋₆) carboxy(C₀₋₆ alkyl);
and wherein R^{3a} and R^{3b}, together with the atoms to which they are attached, may form a saturated ring system, wherein said saturated ring system may be substituted by 0, 1, or 2 R^{3c} substituents;
R^{4a} is selected from hydrogen, C₁₋₆ alkyl, hydroxy, C₁₋₄ alkyloxy, and fluoro;
R^{4b} is selected from bicyclic heteroaryl(C₀₋₃ alkyl), and bicyclic aryl(C₀₋₃ alkyl),
wherein R^{4b} is substituted with 0, 1, or 2 R^{4c} substituents each independently selected from halo, hydroxy, cyano, nitro, carboxy, carboxy(C₁₋₆ alkyl), (C₁₋₆ alkyloxy)carbonyl(C₀₋₆ alkyl), C₁₋₆ alkyloxy, (C₁₋₆ alkyl)oxy(C₁₋₆ alkyl), C₁₋₆ alkyl, and C₁₋₆ haloalkyl;
R^{5a} is selected from hydrogen, C₁₋₁₀ alkyl, C₁₋₁₀ fluoroalkyl, carboxy(C₁₋₁₀ alkyl), hydroxy, hydroxy(C₁₋₁₀ alkyl), cyano(C₁₋₁₀ alkyl), heterocycloalkyl(C₀₋₁₀ alkyl), aminocarbonyl(C₁₋₆ alkyl), (C₁₋₆ alkyl)₀₋₂ aminocarbonyl(C₁₋₆ alkyl), (carboxy(C₁₋₁₀ alkyl))oxy(C₁₋₆ alkyl), aryl(C₀₋₆ alkyl), (C₃₋₁₂)cycloalkyl(C₀₋₆ alkyl), heteroaryl(C₀₋₆ alkyl), (C₁₋₆ alkyl)oxy(C₁₋₆ alkyl), (C₁₋₆ alkyloxy)carbonyl(C₀₋₆ alkyl), amino, amino(C₁₋₆ alkyl), (C₁₋₆ alkyl)₀₋₂ amino(C₀₋₆ alkyl), amino(C₁₋₆ alkyl)carbonylamino(C₁₋₆ alkyl), (C₁₋₆ alkyl)₀₋₂ amino(C₁₋₆ alkyl)carbonylamino(C₁₋₆ alkyl), (C₁₋₆ alkyl)₃N⁺(C₂₋₁₀ alkyl), (C₁₋₆ alkyl)₃N⁺(C₁₋₆ alkyl)carbonylamino(C₁₋₆ alkyl), and C₁₋₁₀ fluoroalkyl, and wherein R^{5a} is substituted by 0, 1, or 2 R^{5d} substituents;
R^{5b} is selected from hydrogen, C₁₋₁₀ alkyl, hydroxy(C₁₋₁₀ alkyl), carboxy(C₁₋₆ alkyl), (C₁₋₆ alkyl)oxy(C₁₋₆ alkyl), aminocarbonyl(C₁₋₆ alkyl), (C₁₋₆ alkyl)₀₋₂ aminocarbonyl(C₁₋₆ alkyl), carboxy(C₁₋₁₀ alkyl)oxy(C₁₋₆ alkyl), cyano(C₁₋₁₀ alkyl), amino(C₁₋₆ alkyl), (C₁₋₆ alkyl)₀₋₂ amino(C₁₋₆ alkyl), amino(C₁₋₆ alkyl)carbonylamino(C₁₋₆ alkyl), (C₁₋₆ alkyl)₀₋₂ amino(C₁₋₆ alkyl)carbonylamino(C₁₋₆ alkyl), (C₁₋₆ alkyl)₃N⁺(C₂₋₆ alkyl)oxy(C₁₋₆ alkyl), (C₁₋₆ alkyl)₃N⁺(C₁₋₆ alkyl)carbonylamino(C₁₋₆ alkyl), (C₁₋₆ alkyl)₃N⁺(C₁₋₆ alkyl), (heterocycloalkyl(C₀₋₁₀ alkyl), (C₃₋₁₂)cycloalkyl(C₀₋₁₀ alkyl), and C₁₋₁₀ haloalkyl, wherein R^{5b} is substituted by 0, 1, 2, or 3 R^{5e} substituents,
wherein R^{5a} and R^{5b}, together with the atoms to which they are attached, form a saturated mono- or bi-cyclic ring, wherein said mono- or bi-cyclic ring is substituted with 0, 1, 2, or 3 R^{5d} and 0, 1, 2, or 3 R^{5e} substituents;
each R^{5d} is independently selected from selected from halo, hydroxy, hydroxy(C₁₋₁₀ alkyl), C₁₋₁₀ alkyl, carboxy, carboxy(C₁₋₆ alkyl), C₁₋₆ alkyloxy, (C₁₋₆ alkyl)oxy(C₁₋₆ alkyl), amino, amino(C₁₋₆ alkyl), (C₁₋₆ alkyl)₀₋₂ amino(C₀₋₆ alkyl), amino(C₁₋₆ alkyl)carbonylamino(C₀₋₆ alkyl), (C₁₋₆ alkyl)₀₋₂ amino(C₁₋₆ alkyl)carbonylamino(C₀₋₆ alkyl), (C₁₋₆ alkyl)₃N⁺(C₀₋₆ alkyl), (C₁₋₆ alkyl)₃N⁺(C₂₋₆ alkyl)oxy(C₀₋₆ alkyl), (C₁₋₆ alkyl)₃N⁺(C₁₋₆ alkyl)carbonylamino(C₀₋₆ alkyl), (carboxy(C₁₋₆ alkyl))oxy(C₁₋₆ alkyl), cyano(C₀₋₆ alkyl), tetrazolyl(C₀₋₆ alkyl), and C₁₋₆ haloalkyl, and two R^{5d} substituents together with the atom they are attached to may join together to form a saturated ring;
each R^{5e} is independently selected from halo, hydroxy, hydroxy(C₁₋₁₀ alkyl), C₁₋₁₀ alkyl, carboxy, carboxy(C₁₋₆ alkyl), C₁₋₆ alkyloxy, (C₁₋₆ alkyl)oxy(C₁₋₆ alkyl), amino, amino(C₁₋₆ alkyl), (C₁₋₆ alkyl)₀₋₂ amino(C₀₋₆ alkyl), amino(C₁₋₆ alkyl)carbonylamino(C₀₋₆ alkyl), (C₁₋₆ alkyl)₀₋₂ amino(C₁₋₆ alkyl)carbonylamino(C₀₋₆ alkyl), (C₁₋₆ alkyl)₃N⁺(C₀₋₆ alkyl), (C₁₋₆ alkyl)₃N⁺(C₂₋₆ alkyl)oxy(C₀₋₆ alkyl), (C₁₋₆ alkyl)₃N⁺(C₁₋₆ alkyl)carbonylamino(C₀₋₆ alkyl), (carboxy(C₁₋₆ alkyl))oxy(C₁₋₆ alkyl), cyano(C₀₋₆ alkyl), tetrazolyl(C₀₋₆ alkyl), and C₁₋₆ haloalkyl, and two R^{5e} substituents together with the atom they are attached to may join together to form a saturated ring;
R^{5c} is hydrogen, C₁₋₄ alkyl, hydroxy(C₁₋₄ alkyl), C₁₋₄ alkyloxy, or (C₁₋₄ alkyl)oxy(C₁₋₄ alkyl);
R^{6a} is selected from hydrogen, hydroxy, amino, C₁₋₄ alkyloxy, and C₁₋₆ alkyl;
R^{6b} is selected from hydrogen, C₁₋₆ alkyl, hydroxy, C₁₋₄ alkyloxy, and fluoro;
R^{6c} is selected from C₁₋₁₀ alkyl, C₁₋₆ alkyloxy, (C₁₋₆ alkyl)oxy(C₁₋₆ alkyl), C₁₋₁₀ haloalkyl, aryl(C₀₋₆ alkyl), heteroaryl(C₀₋₁₀ alkyl), (C₁₋₆ alkyl)SO₂(C₀₋₆ alkyl), hydroxy, hydroxy(C₁₋₆ alkyl), amino(C₀₋₆ alkyl), (C₁₋₆ alkyl)₀₋₂ amino(C₀₋₆ alkyl), aminocarbonyl(C₀₋₆ alkyl), (C₁₋₆ alkyl)₀₋₂ aminocarbonyl(C₀₋₆ alkyl), (C₁₋₆ alkyl)carbonylamino(C₀₋₆ alkyl), (C₁₋₆ alkyl)₀₋₂ aminocarbonylamino(C₀₋₆ alkyl), and carboxy(C₀₋₆ alkyl);
R^{7a} is selected from hydrogen, C₁₋₆ alkyl, hydroxy, C₁₋₄ alkyloxy, and fluoro;
R^{7b} is selected from aryl(C₀₋₆ alkyl), heteroaryl(C₀₋₆ alkyl), and (C₃₋₁₂)cycloalkyl(C₀-6 alkyl),
wherein R^{7b} is substituted by 0, 1, 2, or 3 R^{7c} substituents each independently selected from C₁₋₆ alkyl, C₁₋₁₀ fluoroalkyl, C₁₋₁₀ fluoroalkyloxy, (C₁₋₆ alkyl)₀₋₂ amino(C₀₋₅ alkyl), (C₁₋₆ alkyl)₃N⁺(C₀₋₅ alkyl), carboxy(C₀₋₆ alkyl), (C₁₋₆ alkyloxy)carbonyl(C₀₋₆ alkyl), carboxy(C₁₋₆ alkyl)oxy(C₀₋₆ alkyl), aminocarbonyl(C₀₋₆ alkyl), (C₁₋₆ alkyl)₀₋₂ aminocarbonyl(C₀₋₆ alkyl), hydroxy, hydroxy(C₁₋₆ alkyl), halo, —(C₀₋₅ alkyl)-(S(═O)₂OH), —(C₀₋₅ alkyl)-(S(═O)₂NH₂), amino(C₀₋₆ alkyl)oxy(C₀₋₆ alkyl), (C₁₋₆ alkyl)₀₋₂ amino(C₀₋₆ alkyl)oxy(C₀₋₆ alkyl), (C₁₋₆ alkyl)₃N⁺(C₀₋₆ alkyl)oxy(C₀₋₆ alkyl), aminocarbonylamino(C₀₋₆ alkyl), (C₁₋₆ alkyl)₀₋₂ aminocarbonylamino(C₀₋₆ alkyl), C₁₋₁₀ haloalkyl, C₁₋₁₀ haloalkyloxy, and (C₁₋₆ alkyl)oxy(C₀₋₆ alkyl);
R^{8a} is selected from hydrogen, C₁₋₆ alkyl, hydroxy, C₁₋₄ alkyloxy, C₁₋₆ fluoroalkyl, C₁₋₄ fluoroalkyloxy, and halo;
R^{8b} is selected from bicyclic aryl(C₀₋₃ alkyl) and bicyclic heteroaryl(C₀₋₃ alkyl), wherein R^{8b} is substituted by 0, 1, or 2 R^{8c} substituents each independently selected from C₁₋₄ alkyl, halo, cyano, nitro, carboxy, amino, hydroxy, C₁₋₆ fluoroalkyl, C₁₋₆ fluoroalkyloxy, C₁₋₆ alkyloxy, amino(C₁₋₃ alkyl), and hydroxy(C₁₋₆ alkyl);
R⁹ is selected from hydrogen, and C₁₋₄ alkyl;
R^{10a} is selected from hydrogen, hydroxy, C₁₋₆ alkyl, C₁₋₄ alkyloxy, and fluoro;
R^{10b} is selected from (C₃₋₁₂)cycloalkyl(C₀₋₃ alkyl), aryl(C₀₋₃ alkyl), and heteroaryl(C₀₋₃ alkyl), wherein said heteroaryl contains 1, 2, or 3 nitrogen atoms,
wherein R^{10b} is substituted by 0, 1, or 2 R^{10c} wherein each R^{10c} is independently selected from C₁₋₁₀ alkyl, C₁₋₁₀ fluoroalkyl, C₁₋₆ haloalkyl, C₁₋₁₀ fluoroalkyloxy, amino, amino(C₁₋₆ alkyl), (C₁₋₆ alkyl)₀₋₂ amino(C₀₋₆ alkyl), (C₁₋₆ alkyl)₃N⁺(C₀₋₆ alkyl), hydroxy, hydroxy(C₁₋₆ alkyl), cyano, halo, aminocarbonyl, aminocarbonyl(C₁₋₆ alkyl), (C₁₋₆ alkyl)₀₋₂ aminocarbonyl(C₀₋₆ alkyl), (C₁₋₆ alkyl)₀₋₂ aminocarbonylamino(C₀₋₆ alkyl), (C₁₋₆ alkyl)carbonylamino(C₀₋₆ alkyl), carboxy(C₀₋₆ alkyl), (C₁₋₆ alkoxy)carbonyl(C₀₋₆ alkyl), carboxy(C₁₋₆ alkyl)oxy(C₀₋₆ alkyl), —(C₀₋₅ alkyl)-(S(═O)₂OH), —(C₀₋₅ alkyl)-(S(═O)₂NH₂), amino(C₀₋₆ alkyl)oxy(C₀₋₆ alkyl), (C₁₋₆ alkyl)₀₋₂ amino(C₀₋₆ alkyl)oxy(C₀₋₆ alkyl), (C₁₋₆ alkyl)₃N⁺(C₀₋₆ alkyl)oxy(C₀₋₆ alkyl), (C₁₋₆ alkyl)oxy(C₀₋₆ alkyl), C₁₋₆ haloalkyloxy, (C₃₋₁₂)cycloalkyl(C₀₋₆ alkyl), and heterocycloalkyl(C₀₋₆ alkyl);
R^{11a} is selected from hydrogen, hydroxy, C₁₋₆ alkyl, C₁₋₄ alkyloxy, and fluoro;
R^{11b} is selected from aryl(C₀₋₃ alkyl), heteroaryl(C₀₋₃ alkyl), wherein said heteroaryl contains 1, 2, or 3 nitrogen atoms, and H₂N—C(═NH)NH—(C₁₋₆ alkyl),
wherein R^{11b} is substituted by 0, 1, 2, 3, or 4 R^{11c} substituents each independently selected from C₁₋₆ alkyl, amino, amino(C₁₋₆ alkyl), (C₁₋₆ alkyl)₀₋₂ amino(C₀₋₆ alkyl), (C₁₋₆ alkyl)₃N⁺(C₀₋₆ alkyl), hydroxy, hydroxy(C₁₋₆ alkyl), cyano, halo, aminocarbonyl, aminocarbonyl(C₁₋₆ alkyl), (C₁₋₆ alkyl)₀₋₂ aminocarbonyl(C₀₋₆ alkyl), carboxy(C₀₋₆ alkyl), carboxy(C₁₋₆ alkyl)oxy(C₀₋₆ alkyl), amino(C₀₋₆ alkyl)oxy(C₀₋₆ alkyl), (C₁₋₆ alkyl)₀₋₂ amino(C₀₋₆ alkyl)oxy(C₀₋₆ alkyl), (C₁₋₆ alkyl)₃N⁺(C₀₋₆ alkyl)oxy(C₀₋₆ alkyl), (C₁₋₆ alkyl)oxy(C₀₋₆ alkyl), C₁₋₆ haloalkyloxy, C₁₋₆ haloalkyl, (C₃₋₁₂)cycloalkyl(C₀₋₆ alkyl), heterocycloalkyl(C₀₋₆ alkyl), ((C₁₋₆ alkyl)carbonyl)heterocycloalkyl(C₀₋₁₀ alkyl), and ((C₁₋₆ alkyl)carbonyloxy)heterocycloalkyl(C₀₋₁₀ alkyl);
R^{12a} is selected from hydrogen, hydroxy, amino, C₁₋₁₀ alkyl, and (C₃₋₁₂)cycloalkyl(C₀₋₆ alkyl);
R^{12b} is selected from hydrogen, C₁₋₁₀ alkyl, and (C₃₋₁₂)cycloalkyl(C₀₋₆ alkyl); wherein R^{12a} and R^{12b}, together with the atoms to which they are attached, may form a saturated ring;
R^{13a} is selected from hydrogen, hydroxy, C₁₋₆ alkyl, C₁₋₄ alkyloxy, and fluoro;
R^{13b} is selected from hydrogen, and C₁₋₄ alkyl;
R^{13c} is selected from a bicyclic nitrogen-containing heteroaryl having 1 or 2 nitrogen and bicyclic-aryl and wherein R^{13c} is substituted independently by 0, 1, or 2 R^{13d} substituents each independently selected from halo, C₁₋₆ alkyl, carboxy(C₀₋₄ alkyl), C₁₋₄ haloalkyloxy, and C₁₋₄ alkyloxy;

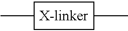

is selected from

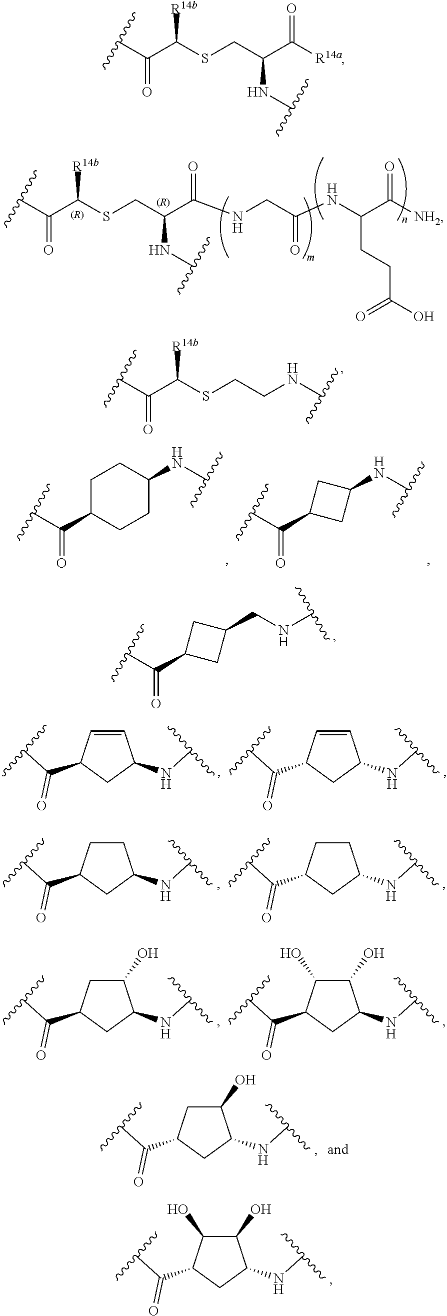

wherein R^{14a} is selected from amino, hydroxy, (C₁₋₆ alkyl)₀₋₂ amino, and C₁₋₆ alkyloxy;
m is selected from 1, 2, 3, or 4;
n is selected from 1, 2, 3, or 4; and
R^{14b} is selected from hydrogen, C₁₋₈ alkyl, aryl(C₀₋₆ alkyl), and heteroaryl(C₀₋₆ alkyl), wherein R^{14b} is substituted by 0, 1, 2, or 3 halo groups.

In a first embodiment of the invention, R¹ is selected from aminomethyl, aminoethyl, aminopropyl, aminobutyl, aminopentyl, phenyl, phenylmethyl, phenylethyl, phenylpropyl, styryl, biphenyl, naphthyl, pyridyl, pyridazinyl, pyrimidyl, pyrazinyl, imidazolyl, pyrazolyl, furyl, thiophenyl, oxazolyl, isoxazolyl, thiazolyl, isothiazolyl, oxadiazolyl, biphenylmethyl, naphthylmethyl, pyridylmethyl, pyridazinylmethyl, pyrimidylmethyl, pyrazinylmethyl, imidazolylmethyl, pyrazolylmethyl, furylmethyl, thiophenylmethyl, oxazolylmethyl, isoxazolylmethyl, thiazolylmethyl, isothiazolylmethyl, oxadiazolylmethyl, bicyclo[1.1.1.]pentyl, (bicyclo[1.1.1.]pentyl)methyl, phenylcarbonylaminoethyl, aminocarbonylmethyl, aminocarbonylethyl, aminocarbonylpropyl, aminocarbonylisopropyl, aminocarbonylbutyl, aminocarbonylaminomethyl, aminocarbonylaminoethyl, aminocarbonylaminopropyl, aminocarbonylaminobutyl, methyl, ethyl, propyl, isopropyl, butyl, sec-butyl, isobutyl, tert-butyl, n-pentyl, isopentyl, neopentyl, n-hexyl, isohexyl, n-heptyl, n-octyl, cyclopropyl, cyclobutyl, cyclopentyl, cyclohexyl, cyclopropylmethyl, cyclobutylmethyl, cyclopentylmethyl, cyclohexylmethyl, adamantyl, carboxy, carboxymethyl, carboxyethyl, azidomethyl, azidoethyl, azidopropyl, phenylaminocarbonylmethyl, pyridylaminocarbonylmethyl, (pyridylcarbonylamino)methyl, guanidino, guanidinomethyl, guanidinoethyl, guanidinopropyl, guanidinobutyl, fluoromethyl, difluoromethyl, trifluoromethyl, fluoroethyl, difluoroethyl, trifluoroethyl, pentafluoroethyl, fluoropropyl, difluoropropyl, trifluoropropyl, pentafluoropropyl, heptafluoropropyl, trifluorobutyl, N,N,N-trimethylmethylammonium, N,N,N-trimethyleth-1-ylammonium, N,N,N-trimethylpropan-1-ylammonium, N,N,N-trimethylbut-1-ylammonium, methylamino, methylaminomethyl, methylaminoethyl, methylaminopropyl, methylaminobutyl, dimethylamino, dimethylaminomethyl, dimethylaminoethyl, dimethylaminopropyl, dimethylaminobutyl, ethylamino, ethylaminomethyl, ethylaminoethyl, ethylaminopropyl, ethylaminobutyl, diethylamino, diethylaminomethyl, diethylaminoethyl, diethylaminopropyl, diethylaminobutyl, aminoethoxy, aminoethoxymethyl, isoxazolylcarbonylaminomethyl, methoxy, ethoxy, propoxy, isopropoxy, cyclopropoxy, cyclopropoxymethyl, cyclopropylmethoxy, cyclopropylmethoxymethyl, cyclopropylmethoxyethyl, methoxymethyl, ethoxymethyl, methoxyethyl, ethoxyethyl, vinyl, prop-2-enyl, but-3-enyl, and pent-4-enyl, and the other groups are as provided in the general Formula (I) above.

In a second embodiment of the invention, R¹ is selected from 3-aminopropyl, 4-aminobutyl, phenyl, phenylmethyl, bicyclo[1.1.1.]pentyl, phenylcarbonylaminoethyl, aminocarbonylmethyl, aminocarbonylaminoethyl, n-propyl, n-butyl, isobutyl, isopentyl, n-pentyl, n-hexyl, 1-azidoethyl, 2-azidoethyl, azidopropyl, 3-azidopropyl, pyridylaminocarbonylmethyl, 3-guanidinopropyl, 2,2-difluoropropyl, 4,4,4-trifluorobutyl, N,N,N-trimethylpropan-1-ylammonium, methylaminopropyl, dimethylaminopropyl, isoxazolylcarbonylaminomethyl, ethoxy, methoxymethyl, prop-2-enyl, cyclopropylmethoxy, and aminoethoxy, and the other groups are as provided in the general Formula (I) above or as in the first embodiment.

In a third embodiment of the invention, R^{1a} substituents are each independently selected from C₁₋₆ alkyl, amino, cyano, halo, and hydroxy, and the other groups are as provided in the general Formula (I) above or as in the first through second embodiments.

In a fourth embodiment of the invention, each R^{2a} is independently selected from hydrogen, hydroxy, methyl, ethyl, methoxy, ethoxy, fluoro, and the other groups are as provided in the general Formula (I) above, or as in the first through third embodiments.

In a fifth embodiment of the invention, each R^{2a} is independently selected from hydrogen, hydroxy, and methyl, and the other groups are as provided in the general Formula (I) above, or as in the first through fourth embodiments.

In a sixth embodiment of the invention, R^{2b} is selected from phenyl, benzyl, biphenyl, naphthyl, pyridyl, pyridazinylpyrimidyl, pyrazinyl, imidazolyl, pyrazolyl, oxazolyl, isoxazolyl, thiazolyl, isothiazolyl, oxadiazolyl, and bicyclo[1.1.1]pentyl, and the other groups are as provided in the general Formula (I) above, or as in the first through fifth embodiments.

In a seventh embodiment, R^{2b} is selected from phenyl, pyridyl, pyrimidyl, pyridazinyl, imidazolyl, and bicyclo[1.1.1]pentyl, and the other groups are as provided in the general Formula (I) above, or as in the first through sixth embodiments.

In an eighth embodiment, each R^{2c} is independently selected from aminomethyl, hydroxy, methoxy, ethoxy, difluoromethoxy, trifluoromethoxy, fluoromethyl, difluoromethyl, trifluoromethyl, trifluoroethyl, methyl, ethyl, n-propyl, isopropyl, cyclopropyl, n-butyl, tert-butyl, fluoro, chloro, bromo, iodo, aminoethoxy, N-methylaminoethoxy, N-ethylaminoethoxy, N,N-dimethylaminoethoxy,

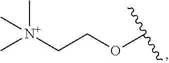 [SMILES: *OCC[N+](C)(C)C]

carboxy, carboxymethoxy, (carboxymethoxy)methyl, aminocarbonyl, N,N-dimethylaminocarbonyl, and aminocarbonylmethyl, and the other groups are as provided in the general Formula (I) above, or as in the first through seventh embodiments.

In a ninth embodiment, each R^{2c} is independently selected from aminomethyl, hydroxy, methoxy, fluoro, carboxy, and aminocarbonyl, and the other groups are as provided in the general Formula (I) above, or as in the first through eighth embodiments.

In a tenth embodiment of the invention, R^{3a} is selected from hydrogen, methyl, ethyl, n-propyl, isopropyl, n-butyl, 2-methylpropyl, tert-butyl, cyclopropyl, cyclopropylmethyl, cyclopropylethyl, cyclobutyl, cyclobutylmethyl, cyclobutylethyl, aminoethyl, aminopropyl, aminobutyl, hydroxyethyl, hydroxypropyl, hydroxybutyl, carboxymethyl, carboxyethyl, carboxypropyl, carboxybutyl, thioethyl, and thiopropyl, wherein R^{3a} may be substituted by 0, 1, or 2 R^{3c} substituents each independently selected from fluoro, chloro, methyl, ethyl, n-propyl, isopropyl, cyclopropyl, n-butyl, sec-butyl, tert-butyl, amino, aminomethyl, N-methylamino, N-methylaminomethyl, N-ethylamino, N-ethylaminomethyl, N,N-dimethylamino, N,N-dimethylaminomethyl, N,N-diethylamino, N,N-diethylaminomethyl, N,N,N-trimethylammonium, N,N,N-trimethylmethylammonium, hydroxy, hydroxymethyl, —SO₂CH₃, —CH₂SO₂CH₃, —CH₂CH₂SO₂CH₃, cyano, cyanomethyl, methoxy, ethoxy, methoxymethyl, methoxyethyl, carboxy, carboxymethyl, and carboxyethyl, and the other groups are as provided in the general Formula (I) above, or as in the first through ninth embodiments.

In a eleventh embodiment of the invention, R^{3a} is selected from hydrogen, methyl, ethyl, propyl, 2-methylpropyl, butyl, aminoethyl, 2-aminoethyl, aminopropyl, 3-aminopropyl, hydroxyethyl, 2-hydroxyethyl, hydroxypropyl, 2-hydroxypropyl, 3-hydroxypropyl, carboxyethyl, 2-carboxyethyl, and thioethyl, wherein R^{3a} may be substituted by 0, 1, or 2 R^{3c} substituents each independently selected from methyl, isopropyl, cyclopropyl, amino, N-methylamino, hydroxy, —SO₂CH₃, —CH₂SO₂CH₃, cyano, methoxy, and carboxy, and the other groups are as provided in the general Formula (I) above, or as in the first through tenth embodiments.

In a twelfth embodiment of the invention, R^{3b} is selected from hydrogen, methyl, ethyl, n-propyl, isopropyl, n-butyl, isobutyl, sec-butyl, tert-butyl, n-pentyl, isopentyl, neopentyl, cyclopropyl, cyclobutyl, cyclopentyl, cyclohexyl, cyclopropylmethyl, cyclobutylmetyl, cyclopentylmethyl, cyclohexylmethyl, bicyclo[1.1.1]pentylmethyl, hydroxymethyl, 1-hydroxyethyl, 2-hydroxyethyl, hydroxypropyl, 3-hydroxypropyl, 1-methyl-1-hydroxyethyl, hydroxyisopropyl, hydroxybutyl, methoxymethyl, methoxyethyl, methoxypropyl, ethoxymethyl, ethoxyethyl, aminomethyl, 2-aminoethyl, N-methylaminomethyl, N,N-dimethylaminomethyl, N-methylaminoethyl, N,N-dimethylaminoethyl, N-methylaminopropyl, N,N-dimethylaminopropyl, 1-aminopropyl, 2-aminopropyl, 3-aminopropyl, 2-aminoprop-2-yl, fluoromethyl, difluoromethyl, trifluoromethyl, fluoroethyl, difluoroethyl, trifluoroethyl, benzyl, 3-pyridinylmethyl, 4-pyridinylmethyl, imidazolylmethyl, thiazolylmethyl, oxazolylmethyl, thiophenylmethyl, furanylmethyl, pyrazolylmethyl, N-pyrazolylmethyl, 1-phenylethyl, 1-(4-pyridinyl)ethyl, aminocarbonylmethyl, aminocarbonylethyl, aminocarbonylpropyl, (N,N-dimethyl)aminocarbonylmethyl, (N,N-dimethyl)aminocarbonylethyl, thiomethyl, thioethyl, thiopropyl, —CH₂CH₂SO₂CH₃, carboxymethyl, carboxyethyl, 2-carboxyethyl, carboxypropyl, 3-carboxypropyl, carboxybutyl, 4-carboxybutyl, piperazinylmethyl, morpholinomethyl, piperidinylmethyl, azetidinylmethyl, tetrahydropyranylmethyl, aminocarbonylaminomethyl, aminocarbonylaminoethyl, aminocarbonylaminopropyl, and aminocarbonylaminobutyl, wherein R^{3b} may be substituted by 0, 1, or 2 R^{3c} substituents each independently selected from fluoro, chloro, methyl, ethyl, n-propyl, isopropyl, cyclopropyl, n-butyl, sec-butyl, tert-butyl, amino, aminomethyl, N-methylamino, N-methylaminomethyl, N-ethylamino, N-ethylaminomethyl, N,N-dimethylamino, N,N-dimethylaminomethyl, N,N-diethylamino, N,N-diethylaminomethyl, N,N,N-trimethylammonium, N,N,N-trimethylmethylammonium, hydroxy, hydroxymethyl, —SO₂CH₃, —CH₂SO₂CH₃, —CH₂CH₂SO₂CH₃, cyano, cyanomethyl, methoxy, ethoxy, methoxymethyl, methoxyethyl, carboxy, carboxymethyl, and carboxyethyl, and the other groups are as provided in the general Formula (I) above, or as in the first through eleventh embodiments.

In a thirteenth embodiment of the invention, R^{3b} is selected from hydrogen, methyl, ethyl, n-propyl, isopropyl, isobutyl, n-butyl, cyclopropyl, cyclobutyl, hydroxymethyl, 1-hydroxyethyl, 2-hydroxyethyl, hydroxypropyl, 3-hydroxypropyl, 1-methyl-1-hydroxyethyl, methoxyethyl, aminomethyl, 2-aminoethyl, N-methylaminomethyl, 1-methyl-1-aminoethyl, 2-aminoprop-2-yl, 1-phenylmethyl, benzyl, imidazolylmethyl, thiazolylmethyl, aminocarbonylmethyl, aminocarbonylethyl, thiomethyl, —CH₂CH₂SO₂CH₃, carboxymethyl, 2-carboxyethyl, 3-carboxypropyl, 4-carboxybutyl, piperazinylmethyl, and aminocarbonylaminopropyl, wherein R^{3b} may be substituted by 0, 1, or 2 R^{3c} substituents each independently selected from methyl, isopropyl, cyclopropyl, amino, N-methylamino, hydroxy,

—SO₂CH₃, —CH₂SO₂CH₃, cyano, methoxy, and carboxy, and the other groups are as provided in the general Formula (I) above, or as in the first through twelfth embodiments.

In a fourteenth embodiment of the invention, R^{3a} and R^{3b}, together with the atoms to which they are attached, form a saturated ring system substituted by 0, 1, or 2, R^{3c} substituents and the ring system is selected from:

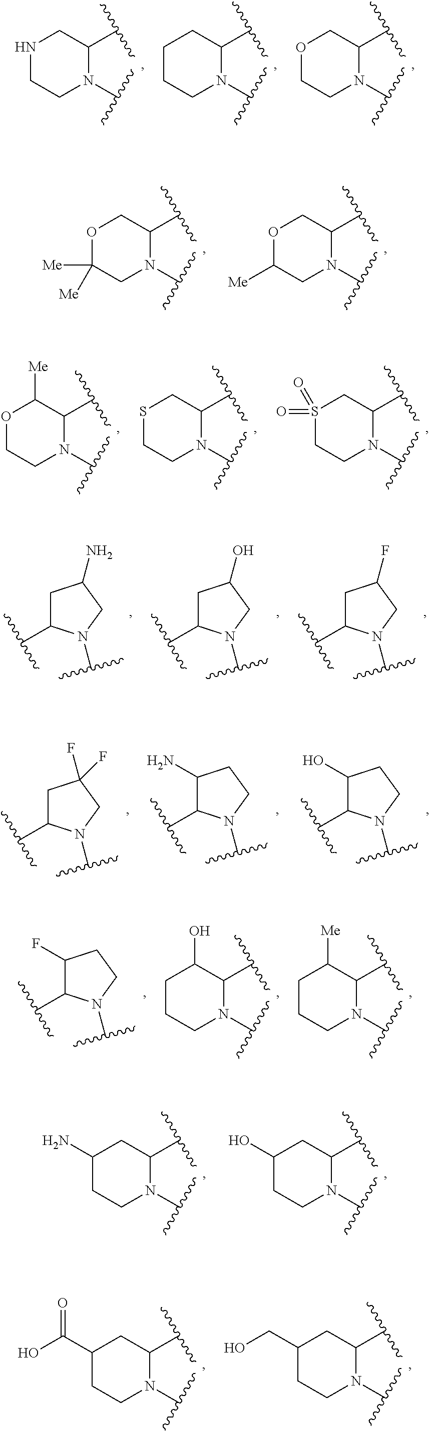

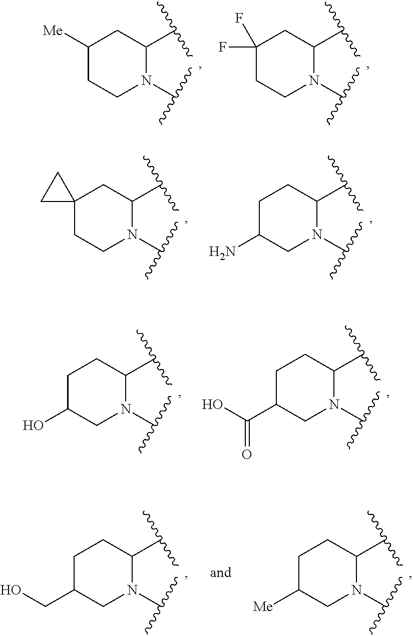

and Me, and the other groups are as provided in the general Formula (I) above, or as in the first through thirteenth embodiments.

In a fifteenth embodiment of the invention, R^{3a} and R^{3b}, together with the atoms to which they are attached, form a saturated ring system substituted by 0, 1, or 2, R^{3c} substituents and the ring system is selected from:

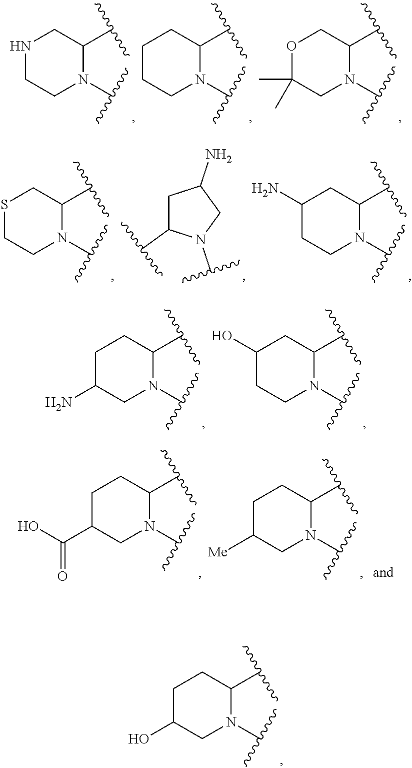

and the other groups are as provided in the general Formula (I) above, or as in the first through fourteenth embodiments.

In a sixteenth embodiment of the invention, R^{4a} is selected from hydrogen, methyl, ethyl, propyl, hydroxy, methoxy, and fluoro, and the other groups are as provided in the general Formula (I) above, or as in the first through fifteenth embodiments.

In a seventeenth embodiment of the invention, R^{4a} is hydrogen or methyl, and the other groups are as provided in the general Formula (I) above, or as in the first through sixteenth embodiments.

In an eighteenth embodiment of the invention, R^{4b} is selected from indolyl, naphthyl, quinolinyl, pyrrolo[2,3-b]pyridinyl, [1,2,4]triazolo[1,5-a]pyridine, 1H-pyrazolo[3,4-b]pyridine, indazolyl, benzothiazolyl, and benzothiophenyl, wherein R^{4b} substituted with 0, 1, or 2 R^{4c} substituents each independently selected from fluoro, chloro, bromo, iodo, cyano, nitro, hydroxy, methyl, ethyl, propyl, isopropyl, carboxy, carboxymethyl, and carboxyethyl, and the other groups are as provided in the general Formula (I) above, or as in the first through seventeenth embodiments.

In a nineteenth embodiment of the invention, R^{4b} is selected from indolyl, naphthyl, quinolinyl, pyrrolo[2,3-b]pyridinyl, 1H-pyrazolo[3,4-b]pyridine, and indazolyl, wherein R^{4b} substituted with 0, 1, or 2 R^{4c} substituents each independently selected from fluoro, chloro, methyl, and carboxymethyl, and the other groups are as provided in the general Formula (I) above, or as in the first through eighteenth embodiments.

In a twentieth embodiment of the invention, R^{5a} is selected from hydrogen, methyl, ethyl, n-propyl, isopropyl, cyclopropyl, n-butyl, isobutyl, cyclobutyl, n-pentyl, isopentyl, neopentyl, cyclopentyl, cyclobutyl, cyclopropylmethyl, cyclopropylethyl, cyclobutylmethyl, cyclobutylethyl, cyclopentylmethyl, cyclopentylethyl, cyclohexylmethyl, cyclohexylethyl, phenyl, benzyl, phenylethyl, phenylpropyl, oxazolylmethyl, thiazolylmethyl, imidazolylmethyl, triazolylmethyl, oxadiazolylmethyl, thiadiazolylmethyl, oxazolylethyl, thiazolylethyl, imidazolylethyl, triazolylethyl, oxadiazolylethyl, thiadiazolylethyl, oxazolylpropyl, thiazolylpropyl, imidazolylpropyl, triazolylpropyl, oxadiazolylpropyl, thiadiazolylpropyl, azetidinylmethyl, azetidinylethyl, oxetanylmethyl, oxetanylmethyl, pyrrolidinylmethyl, pyrrolidinylethyl, tetrahydrofuranylmethyl, tetrahydrofuranylethyl, piperidinylmethyl, piperidinylethyl, piperazinylmethyl, piperazinylethyl, tetrahydropyranylmethyl, tetrahydropyranylmethyl, hydroxyethyl, hydroxypropyl, hydroxyisopropyl, hydroxybutyl, 3-hydroxy-2,2-dimethylpropyl, cyclopropylmethyl, 1-hydroxypropan-2-yl, 2-hydroxyethyl, 3-hydroxypropyl, 2-hydroxyisopropyl, methoxyethyl, methoxypropyl, ethoxyethyl, ethoxypropyl, cyanomethyl, cyanoethyl, cyanopropyl, cyanobutyl, carboxymethyl, carboxyethyl, carboxypropyl, carboxybutyl, 2-fluoroethyl, 2,2-difluoroethyl, 2,2,2-trifluoroethyl, 2-aminoethyl, 3-aminopropyl, 3-amino-2,2-dimethylpropyl, cyclopropylmethyl, 4-aminobutyl, aminomethylcarbonylaminoethyl, aminoethylcarbonylaminoethyl, aminomethylcarbonylaminopropyl, aminoethylaminocarbonylmethyl, aminoethylaminocarbonylethyl, aminoethylaminocarbonylpropyl, aminohexylcarbonylaminoethyl, aminohexylcarbonylaminoethyl, (N-methylamino)ethyl, (N-methylamino)propyl, (N-ethylamino)ethyl, (N,N-diethylamino)propyl, (N,N-dimethylamino)ethyl, (N,N-dimethylamino)propyl, (N,N-diethylamino)ethyl, (N,N-diethylamino)propyl, (N,N,N-trimethylammonium)ethyl, (N,N,N-trimethylammonium)propyl, (N,N,N-triethylammonium)ethyl, (N,N,N-triethylammonium)propyl, (N-methylamino)methylcarbonylaminoethyl, (N-methylamino)ethylcarbonylaminoethyl, (N-methylamino)methylcarbonylaminopropyl, (N-ethylamino)methylcarbonylaminoethyl, (N-ethylamino)ethylcarbonylaminoethyl, (N-ethylamino)methylcarbonylaminopropyl, (N-methylamino)pentylcarbonylaminoethyl, (N-methylamino)pentylcarbonylaminoethyl, (N-methylamino)pentylcarbonylaminopropyl, (N,N-dimethylamino)methylcarbonylaminoethyl, (N,N-dimethylamino)ethylcarbonylaminoethyl, (N,N-dimethylamino)methylcarbonylaminopropyl, (N,N-diethylamino)methylcarbonylaminoethyl, (N,N-diethylamino)ethylcarbonylaminoethyl, (N,N-diethylamino)methylcarbonylaminopropyl, (N,N-dimethylamino)pentylcarbonylaminoethyl, (N,N-dimethylamino)pentylcarbonylaminoethyl, (N,N-dimethylamino)pentylcarbonylaminopropyl, N,N,N-trimethyl-ethan-1-ammonium, N,N,N-trimethyl-propan-1-ammonium, (N,N,N-trimethylammonium)methylcarbonylaminoethyl, (N,N,N-trimethylammonium)ethylcarbonylaminoethyl, (N,N,N-trimethylammonium)methylcarbonylaminopropyl, (N,N,N-trimethylammonium)ethylcarbonylaminopropyl, (N,N,N-trimethylammonium)pentylcarbonylaminoethyl, (N,N,N-trimethylammonium)pentylcarbonylaminopropyl, (carboxymethyl)oxyethyl, and (carboxymethyl)oxypropyl, wherein R^{5a} is substituted by 0, 1, 2, or 3 R^{5d} substituents each independently selected from chloro, fluoro, hydroxy, hydroxymethyl, hydroxyethyl, methyl, ethyl, n-propyl, isopropyl, n-butyl, isobutyl, cyclobutyl, n-pentyl, isopentyl, neopentyl, carboxy, carboxymethyl, carboxyethyl, methoxy, ethoxy, methoxymethyl, methoxyethyl, methoxypropyl, amino, aminomethyl, aminoethyl, N-methylamino, (N-methylamino)methyl, (N-methylamino)ethyl, N,N-dimethylamino, (N,N-dimethylamino)methyl, (N,N-dimethylamino)ethyl, N,N-diethylamino, (N,N-diethylamino)methyl, (N,N-diethylamino)ethyl, aminomethylcarbonylamino, aminoethylcarbonylamino, aminopentylcarbonylamino, aminomethylcarbonylaminomethyl, aminoethylcarbonylaminomethyl, (N-methylamino)methylcarbonylamino, (N-methylamino)ethylcarbonylamino, (N-methylamino)methylcarbonylaminomethyl, (N-methylamino)ethylcarbonylaminomethyl, (N,N-dimethylamino)methylcarbonylamino, (N,N-dimethylamino)ethylcarbonylamino, (N,N-dimethylamino)methylcarbonylaminomethyl, (N,N-dimethylamino)ethylcarbonylaminomethyl, (N,N-diethylamino)methylcarbonylamino, (N,N-diethylamino)ethylcarbonylamino, (N,N-diethylamino)methylcarbonylaminomethyl, (N,N-diethylamino)ethylcarbonylaminomethyl, N,N,N-trimethylammonium, (N,N,N-trimethylammonium)methyl, (N,N,N-trimethylammonium)ethyl, (N,N,N-trimethylammonium)ethoxy, (N,N,N-trimethylammonium)ethoxymethyl, (N,N,N-trimethylammonium)methylcarbonylamino, (N,N,N-triethylammonium)methylcarbonylamino, (N,N,N-trimethylammonium)ethylcarbonylamino, (N,N,N-trimethylammonium)pentylcarbonylamino, (N,N,N-trimethylammonium)methylcarbonylaminomethyl, (N,N,N-trimethylammonium)ethylcarbonylaminomethyl, cyano, cyanomethyl, cyanoethyl, tetrazoyl, tetrazoylmethyl, tetrazoylethyl, carboxymethoxy, carboxyethoxy, carboxymethoxymethyl, and carboxyethoxymethyl, and the other groups are as provided in the general Formula (I) above, or as in the first through nineteenth embodiments.

In a twenty-first embodiment of the invention, R^{5a} is selected from hydrogen, methyl, ethyl, n-propyl, cyclopropylmethyl, (1,3,4-oxadiazol-2-yl)ethyl, 2-hydroxyethyl, 3-hydroxypropyl, carboxymethyl, 2-carboxyethyl, 3-carboxypropyl, 2-hydroxyethyl, 2-hydroxy-1-methylethyl, hydroxypropyl, 3-hydroxy-2,2-dimethylpropyl, methoxyethyl, methoxypropyl, aminocarboxyethyl, 2-fluoroethyl, 2,2-difluoroethyl, carboxymethoxyethyl, 2-aminoethyl, 3-aminopropyl, (N,N-dimethylamino)ethyl, N,N,N-trimethyl-ethan-1-ammonium, (N,N,N-trimethylammonium)ethoxyethyl, (N,N,N-trimethylammonium)methylcarbonylaminoethyl, and (carboxymethyl)oxyethyl, wherein R^{5a} is substituted by 0, 1, 2, or 3 R^{5d} substituents each independently selected from fluoro, hydroxy, hydroxymethyl, methyl, carboxy, carboxymethyl, methoxy, amino, N,N-dimethylamino, N,N,N-trimethylammonium, (N,N,N-trimethylammonium)ethoxy, (N,N,N-trimethylammonium)methylcarbonylamino, cyano, tetrazoyl, and carboxymethoxy, and the other groups are as provided in the general Formula (I) above, or as in the first through twentieth embodiments.

In a twenty-second embodiment of the invention, R^{5b} is selected from hydrogen, methyl, ethyl, isopropyl, n-propyl, cyclopropyl, isobutyl, n-butyl, sec-butyl, isobutyl, tert-butyl, cyclobutyl, cyclopropylmethyl, oxetanylmethyl, tetrahydrofurylmethyl, tetrahydropyranylmethyl, hydroxymethyl, hydroxyethyl, hydroxypropyl, methoxymethyl, methoxyethyl, methoxypropyl, carboxymethyl, carboxyethyl, carboxypropyl, carboxybutyl, (carboxymethoxy)methyl, (carboxymethoxy)ethyl, (carboxyethoxy)methyl, (carboxyethoxy)ethyl, aminomethyl, aminoethyl, aminopropyl, aminobutyl, (N-methylamino)methyl, (N-methylamino)ethyl, (N-methylamino)propyl, (N-methylamino)butyl, (N,N-dimethylamino)methyl, (N,N-dimethylamino)ethyl, (N,N-dimethylamino)propyl, (N,N-dimethylamino)butyl, (N,N-diethylamino)methyl, (N,N-diethylamino)ethyl, (N,N-diethylamino)propyl, (N,N-diethylamino)butyl, N,N,N-trimethylmethan-1-ylammonium, N,N,N-trimethylethan-1-ylammonium, N,N,N-triethylethan-1-ylammonium, N,N,N-trimethylpropan-1-ylammonium, N,N,N-trimethylbutan-1-ylammonium, (N,N,N-trimethylammonium)ethoxymethyl, (N,N,N-trimethylammonium)ethoxyethyl, (N,N,N-triethylammonium)ethoxyethyl, (N,N,N-trimethylammonium)ethoxypropyl, (N,N,N-trimethylammonium)ethoxybutyl, aminocarbonylmethyl, aminocarbonylethyl, aminocarbonylpropyl, aminocarbonylbutyl, (N-methylamino)carbonylmethyl, (N-methylamino)carbonylethyl, (N-methylamino)carbonylpropyl, (N-methylamino)carbonylbutyl, (N,N-dimethylamino)carbonylmethyl, (N,N-dimethylamino)carbonylethyl, (N,N-dimethylamino)carbonylpropyl, (N,N-dimethylamino)carbonylbutyl, aminomethylcarbonylaminoethyl, aminomethylcarbonylaminopropyl, aminoethylcarbonylaminoethyl, aminoethylcarbonylaminopropyl, (N,N-dimethylamino)methylcarbonylaminoethyl, (N,N-dimethylamino)methylcarbonylaminopropyl, (N,N-dimethylamino)ethylcarbonylaminoethyl, (N,N-dimethylamino)ethylcarbonylaminopropyl, (N,N,N-trimethylammonium)methylcarbonylaminoethyl, (N,N,N-triethylammonium)methylcarbonylaminoethyl, (N,N,N-trimethylammonium)methylcarbonylaminopropyl, (N,N,N-trimethylammonium)ethylcarbonylaminoethyl, (N,N,N-trimethylammonium)ethylcarbonylaminopropyl, (N,N,N-trimethylammonium)ethoxyethyl, (N,N,N-trimethylammonium)ethoxypropyl, 2-fluoroethyl, 2,2-difluoroethyl, 2,2,2-trifluoroethyl, 3,3,3-trifluoropropyl, cyanomethyl, cyanoethyl, cyanopropyl, cyanoisopropyl, and cyanobutyl, wherein R^{5b} is substituted by 0, 1, 2, or 3 R^{5e} substituents each independently selected from chloro, fluoro, hydroxy, hydroxymethyl, hydroxyethyl, methyl, ethyl, n-propyl, isopropyl, n-butyl, isobutyl, cyclobutyl, n-pentyl, isopentyl, neopentyl, carboxy, carboxymethyl, carboxyethyl, methoxy, ethoxy, methoxymethyl, methoxyethyl, methoxypropyl, amino, aminomethyl, aminoethyl, N-methylamino, (N-methylamino)methyl, (N-methylamino)ethyl, N,N-dimethylamino, (N,N-dimethylamino)methyl, (N,N-dimethylamino)ethyl, N,N-diethylamino, (N,N-diethylamino)methyl, (N,N-diethylamino)ethyl, aminomethylcarbonylamino, aminoethylcarbonylamino, aminopentylcarbonylamino, aminomethylcarbonylaminomethyl, aminoethylcarbonylaminomethyl, (N-methylamino)methylcarbonylamino, (N-methylamino)ethylcarbonylamino, (N-methylamino)methylcarbonylaminomethyl, (N-methylamino)ethylcarbonylaminomethyl, (N,N-dimethylamino)methylcarbonylamino, (N,N-dimethylamino)ethylcarbonylamino, (N,N-dimethylamino)methylcarbonylaminomethyl, (N,N-dimethylamino)ethylcarbonylaminomethyl, (N,N-diethylamino)methylcarbonylamino, (N,N-diethylamino)ethylcarbonylamino, (N,N-diethylamino)methylcarbonylaminomethyl, (N,N-diethylamino)ethylcarbonylaminomethyl, N,N,N-trimethylammonium, (N,N,N-trimethylammonium)methyl, (N,N,N-trimethylammonium)ethyl, (N,N,N-trimethylammonium)ethoxy, (N,N,N-trimethylammonium)ethoxymethyl, (N,N,N-trimethylammonium)methylcarbonylamino, (N,N,N-triethylammonium)methylcarbonylamino, (N,N,N-trimethylammonium)ethylcarbonylamino, (N,N,N-trimethylammonium)pentylcarbonylamino, (N,N,N-trimethylammonium)methylcarbonylaminomethyl, (N,N,N-trimethylammonium)ethylcarbonylaminomethyl, cyano, cyanomethyl, cyanoethyl, tetrazoyl, tetrazoylmethyl, tetrazoylethyl, carboxymethoxy, carboxyethoxy, carboxymethoxymethyl, and carboxyethoxymethyl, and the other groups are as provided in the general Formula (I) above, or as in the first through twenty-first embodiments.

In a twenty-third embodiment of the invention, R^{5b} is selected from hydrogen, methyl, ethyl, n-propyl, cyclopropylmethyl, hydroxymethyl, 2-hydroxyethyl, 2-carboxyethyl, 3-carboxypropyl, 2-aminoethyl, (N,N-dimethylamino)ethyl, 2-methoxyethyl, N,N,N-trimethylethan-1-ammonium, (N,N,N-trimethyl-ammonium)methylcarbonylaminoethyl, (N,N,N-trimethyl-ammonium)ethoxyethyl, 2-fluoroethyl, 2,2-difluoroethyl, and (carboxymethoxy)ethyl, wherein R^{5b} is substituted by 0, 1, 2, or 3 R^{5e} substituents each independently selected from fluoro, hydroxy, hydroxymethyl, methyl, carboxy, carboxymethyl, methoxy, amino, N,N-dimethylamino, N,N,N-trimethylammonium, (N,N,N-trimethylammonium)ethoxy, (N,N,N-trimethylammonium)methylcarbonylamino, cyano, tetrazoyl, and carboxymethoxy, and the other groups are as provided in the general Formula (I) above, or as in the first through twenty-second embodiments.

In a twenty-fourth embodiment of the invention, R^{5c} is hydrogen, methyl, or ethyl, and the other groups are as provided in the general Formula (I) above, or as in the first through twenty-third embodiments.

In a twenty-fifth embodiment of the invention, R^{5c} is hydrogen, or methyl, and the other groups are as provided in the general Formula (I) above, or as in the first through twenty-fourth embodiments.

In a twenty-sixth embodiment of the invention, R^{5a} and R^{5b}, together with the atoms to which they are attached, form a saturated mono- or bi-cyclic ring system substituted with 0, 1, 2, or 3 R^{5d} and 0, 1, 2, or 3 R^{5e} substituents, wherein said mono- or bi-cyclic ring system is selected from:

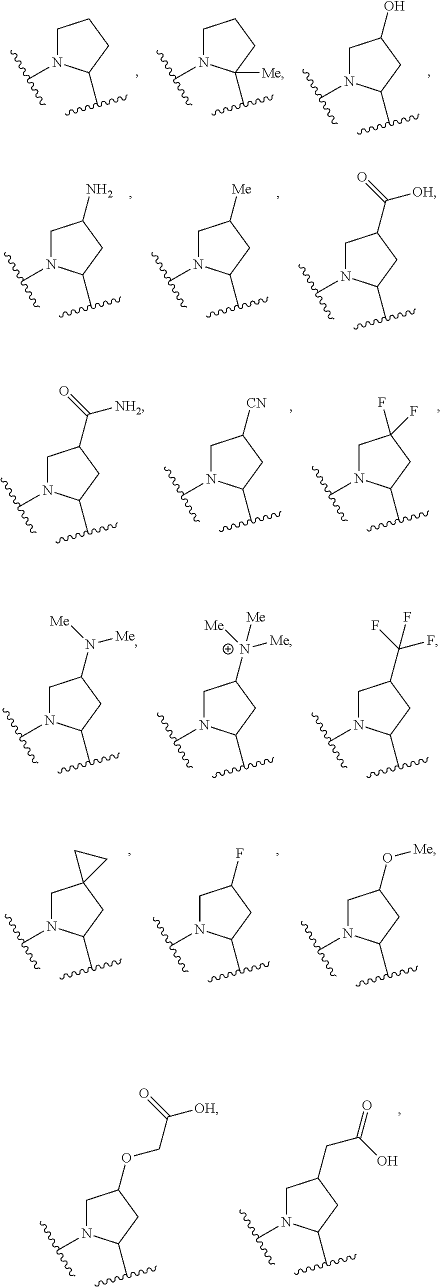

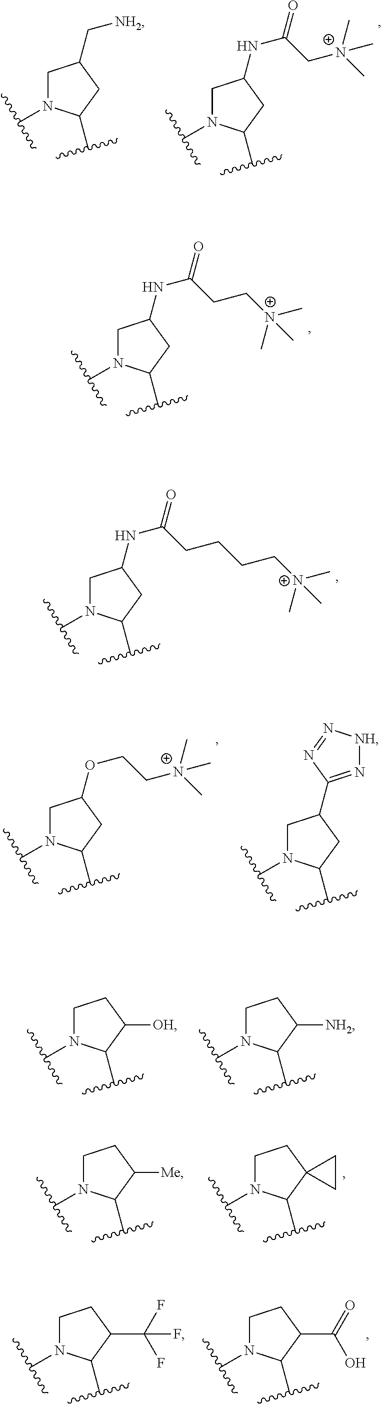

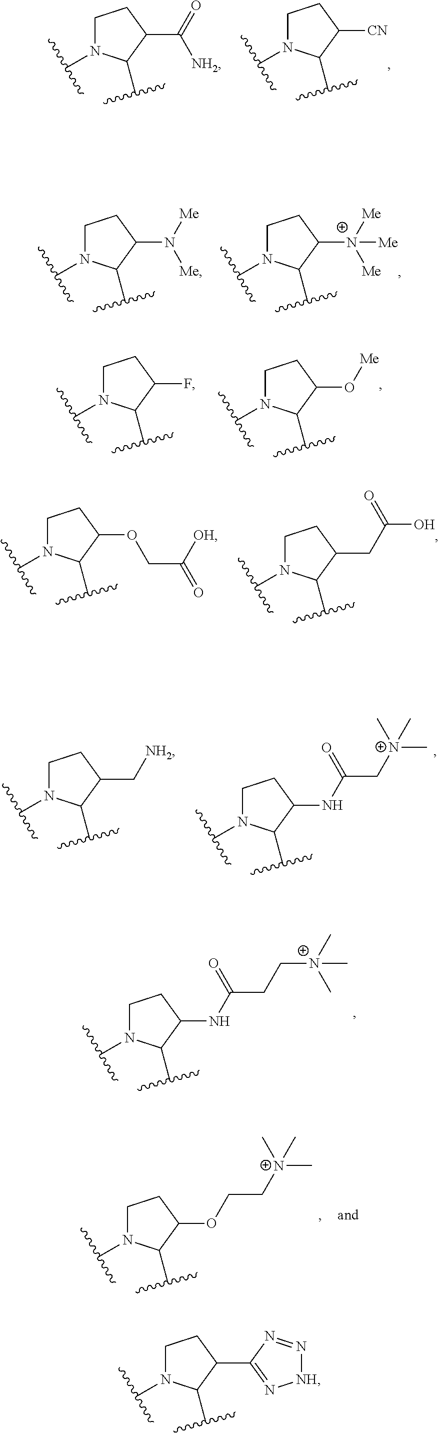

and the other groups are as provided in the general Formula (I) above, or as in the first through the sixteenth and the twenty-first through the twenty-fifth embodiments.

In a twenty-seventh embodiment of the invention, R^{5a} and R^{5b}, together with the atoms to which they are attached, form a saturated mono- or bi-cyclic ring system substituted with 0, 1, 2, or 3 R^{5d} and 0, 1, 2, or 3 R^{5e} substituents, wherein said mono- or bi-cyclic ring system is selected from:

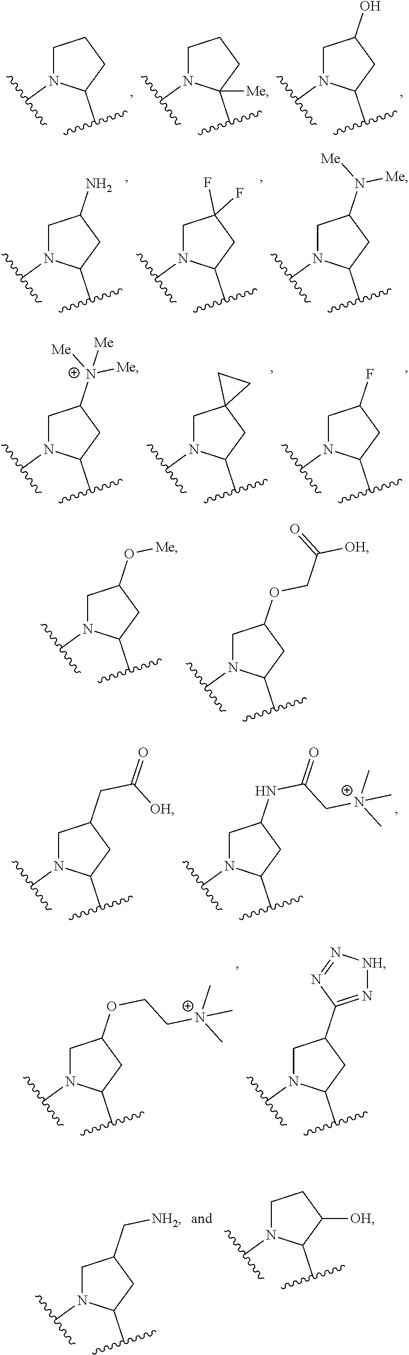

and, d the other groups are as provided in the general Formula (I) above, or as in the first through the sixteenth and the twenty-first through the twenty-sixth embodiments.

In a twenty-eighth embodiment, R^{6a} is selected from hydrogen, hydroxy, methyl, ethyl, propyl, and methoxy, and the other groups are as provided in the general Formula (I) above, or as in the first through the twenty-seventh embodiments.

In a twenty-ninth embodiment, R^{6a} is selected from hydrogen and methyl, and the other groups are as provided in the general Formula (I) above, or as in the first through the twenty-eighth embodiments.

In a thirtieth embodiment, R^{6b} is selected from hydrogen, hydroxy, methyl, ethyl, propyl, methoxy, and ethoxy, and the other groups are as provided in the general Formula (I) above, or as in the first through the twenty-ninth embodiments.

In a thirty-first embodiment, R^{6b} is selected from hydrogen, hydroxy, and methyl, and the other groups are as provided in the general Formula (I) above, or as in the first through the thirtieth embodiments.

In a thirty-second embodiment, R^{6c} is selected from aminocarbonyl, (aminocarbonyl)methyl, (aminocarbonyl)ethyl, (aminocarbonyl)propyl, (N-methylamino)carbonyl, (N-methylamino)carbonylmethyl, (N-methylamino)carbonylethyl, (N-methylamino)carbonylpropyl, (N,N-dimethylamino)carbonyl, (N,N-dimethylamino)carbonylmethyl, (N,N-dimethylamino)carbonylethyl, (N,N-dimethylamino)carbonylpropyl, (N,N-diethylamino)carbonyl, (N,N-diethylamino)carbonylmethyl, (N,N-diethylamino)carbonylethyl, (N,N-diethylamino)carbonylpropyl, aminocarbonylamino, (aminocarbonylamino)methyl, (aminocarbonylamino)ethyl, (aminocarbonylamino)propyl, methoxy, methoxymethyl, methoxyethyl, ethoxy, ethoxymethyl, ethoxyethyl, methylsulfonyl, (methylsulfonyl)methyl, (methylsulfonyl)ethyl, (methylsulfonyl)propyl, amino, aminomethyl, aminoethyl, aminopropyl, aminoisopropyl, aminobutyl, carboxy, carboxymethyl, carboxyethyl, hydroxy, hydroxymethyl, hydroxyethyl, hydroxypropyl, hydroxyisopropyl, trifluoromethyl, 2,2,2-trifluoroethyl, methyl, ethyl, isopropyl, n-propyl, isobutyl, n-butyl, sec-butyl, isobutyl, tert-butyl, phenyl, and benzyl, and the other groups are as provided in the general Formula (I) above, or as in the first through the thirty-first embodiments.

In a thirty-third embodiment, R^{6c} is selected from aminocarbonyl, (aminocarbonyl)methyl, (N,N-dimethylamino)carbonylmethyl, aminocarbonylamino, (aminocarbonylamino)ethyl, methoxymethyl, (methylsulfonyl)methyl, hydroxy, and methyl, and the other groups are as provided in the general Formula (I) above, or as in the first through the thirty-second embodiments.

In a thirty-fourth embodiment, R^{7a} is selected from hydrogen, methyl, ethyl, hydroxy, methoxy, and ethoxy, and the other groups are as provided in the general Formula (I) above, or as in the first through the thirty-third embodiments.

In a thirty-fifth embodiment, R^{7a} is selected from hydrogen, methyl, and hydroxy, and the other groups are as provided in the general Formula (I) above, or as in the first through the thirty-fourth embodiments.

In a thirty-sixth embodiment, R^{7b} is selected from phenyl, biphenyl, naphthyl, pyridyl, pyridazinyl, pyrimidyl, pyrazinyl, imidazolyl, pyrazolyl, furyl, thiophenyl, oxazolyl, isoxazolyl, thiazolyl, isothiazolyl, oxadiazolyl, 3-oxoisoindolinyl, and bicyclo[1.1.1]pentyl, and the other groups are as provided in the general Formula (I) above, or as in the first through the thirty-fifth embodiments.

In a thirty-seventh embodiment, R^{7b} is selected from phenyl, pyridyl, pyrimidyl, furyl, thiazolyl, and 3-oxoisoindolinyl, and the other groups are as provided in the general Formula (I) above, or as in the first through the thirty-sixth embodiments.

In a thirty-eighth embodiment of the invention, R^{7c} is selected from fluoro, chloro, bromo, iodo, hydroxy, methoxy, ethoxy, difluoromethoxy, trifluoromethoxy, carboxy, carboxymethyl, methoxycarbonyl, methoxycarbonylmethyl, ethoxycarbonyl, carboxymethoxy, carboxyethoxy, carboxymethoxymethyl, aminocarbonyl, aminocarbonylmethyl, (N,N-dimethylamino)carbonyl, (N,N-dimethylamino)carbonylmethyl, amino, aminomethyl, N,N-dimethylamino, (N,N-dimethylamino)methyl, (N,N-diethylamino)methyl, N,N,N-trimethylammonium, N,N,N-trimethylmethylammonium, N,N,N-triethylmethylammonium, methyl, ethyl, propyl, isopropyl, difluoromethyl, trifluoromethyl, —SO₂OH, —CH₂SO₂OH, —SO₂NH₂, —CH₂SO₂NH₂, aminoethoxy, aminoethoxymethyl, (N,N-dimethylamino)ethoxy, N,N,N-trimethyleth-1-oxy-ammonium, aminocarbonylamino, and aminocarbonylaminomethyl, and the other groups are as provided in the general Formula (I) above, or as in the first through the thirty-seventh embodiments.

In a thirty-ninth embodiment of the invention, R^{7c} is selected from hydroxy, carboxymethoxy, fluoro, chloro, methoxy, carboxy, carboxymethyl, methoxycarbonyl, aminocarbonyl, aminomethyl, —SO₂OH, —SO₂NH₂, —CH₂SO₂OH, aminoethoxy, and aminocarbonylamino, and the other groups are as provided in the general Formula (I) above, or as in the first through the thirty-eighth embodiments.

In a fortieth embodiment, R^{8a} is selected from hydrogen, methyl, ethyl, hydroxy, methoxy, and ethoxy, and the other groups are as provided in the general Formula (I) above, or as in the first through the thirty-ninth embodiments.

In a forty-first embodiment, R^{8a} is selected from hydrogen, and methyl, and the other groups are as provided in the general Formula (I) above, or as in the first through the fortieth embodiments.

In a forty-second embodiment of the invention, R^{8b} is selected from indolyl, naphthyl, indolylmethyl, naphthylmethyl, quinolinyl, pyrrolo[2,3-b]pyridinyl, indazolyl, benzothiazolyl, and benzothiophenyl, and the other groups are as provided in the general Formula (I) above, or as in the first through the forty-first embodiments.

In a forty-third embodiment of the invention, R^{8b} is selected from indolyl, naphthyl, and pyrrolo[2,3-b]pyridinyl, and the other groups are as provided in the general Formula (I) above, or as in the first through the forty-second embodiments.

In a forty-fourth embodiment of the invention, each R^{8c} independently is selected from methyl, ethyl, isopropyl, fluoro, chloro, bromo, iodo, cyano, amino, aminomethyl, nitro, hydroxy, hydroxymethyl, carboxy, fluoromethyl, difluoromethyl, trifluoromethyl, trichloromethyl, methoxy, ethoxy, and trifluoromethoxy, and the other groups are as provided in the general Formula (I) above, or as in the first through the forty-third embodiments.

In a forty-fifth embodiment of the invention, each R^{8c} independently is selected from fluoro, chloro, bromo, and cyano, and the other groups are as provided in the general Formula (I) above, or as in the first through the forty-fourth embodiments.

In a forty-sixth embodiment of the invention, R⁹ is selected from hydrogen, methyl, ethyl, and propyl, and the other groups are as provided in the general Formula (I) above, or as in the first through the forty-fifth embodiments.

In a forty-seventh embodiment of the invention, R⁹ is hydrogen or methyl, and the other groups are as provided in the general Formula (I) above, or as in the first through the forty-sixth embodiments.

In a forty-eighth embodiment of the invention, R^{10a} is selected from hydrogen, hydroxy, methyl, ethyl, propyl, methoxy, ethoxy, and propoxy, and the other groups are as provided in the general Formula (I) above, or as in the first through the forty-seventh embodiments.

In a forty-ninth embodiment of the invention, R^{10a} is selected from hydrogen, and hydroxy, and the other groups are as provided in the general Formula (I) above, or as in the first through the forty-eighth embodiments.

In a fiftieth embodiment of the invention, R^{10b} is selected from phenyl, benzyl, biphenyl, biphenylmethyl, pyridyl, pyridylmethyl, pyridazinyl, pyrimidyl, pyrazinyl, imidazolyl, imidazolylmethyl, pyrazolyl, furyl, furylmethyl, oxazolyl, oxazolylmethyl, thiazolyl, indolyl, [1,2,4]triazolo[1,5-a]pyridine, and bicyclo[1.1.1]pentyl, and the other groups are as provided in the general Formula (I) above, or as in the first through the forty-ninth embodiments.

In a fifty-first embodiment of the invention, R^{10b} is selected from phenyl, and pyrimidyl, and the other groups are as provided in the general Formula (I) above, or as in the first through the fiftieth embodiments.

In a fifty-second embodiment, each R^{10c} is independently selected from methyl, ethyl, n-propyl, isopropyl, cyclopropyl, n-butyl, isobutyl, cyclobutyl, n-pentyl, isopentyl, neopentyl, cyclopropylmethyl, cyclopropylethyl, cyclobutylmethyl, cyclopentyl, cyclohexyl, cyclopentylmethyl, cyclohexylmethyl, fluoromethyl, difluoromethyl, trifluoromethyl, 2,2,2-trifluoroethyl, trifluoromethoxy, 2,2,2-trifluoroethoxy, fluoro, chloro, bromo, iodo, hydroxy, hydroxymethyl, hydroxyethyl, cyano, amino, aminomethyl, aminoethyl, N-methylamino, (N-methylamino)methyl, (N-methylamino)ethyl, N,N-dimethylamino, (N,N-dimethylamino)methyl, (N,N-dimethylamino)ethyl, N,N,N-trimethylammonium, N,N,N-trimethylmethan-ylammonium, aminocarbonyl, aminocarbonylmethyl, aminocarbonylethyl, (N-methylamino)carbonyl, (N-methylamino)carbonylmethyl, (N,N-dimethylamino)carbonyl, (N,N-dimethylamino)carbonylmethyl, aminocarbonylamino, aminocarbonylaminomethyl, methylcarbonylamino, methylcarbonylaminomethyl, carboxy, carboxymethyl, carboxyethyl, methoxycarboxy, carboxymethoxy, carboxyethoxy, carboxymethoxymethyl, carboxyethoxymethyl, —S(═O)₂OH, —CH₂(S(═O)₂OH), —S(═O)₂NH₂, —CH₂(S(═O)₂NH₂), aminoethoxy, aminopropoxy, (N-methylamino)ethoxy, (N-ethylamino)ethoxy, (N,N-dimethylamino)ethoxy, (N,N-diethylamino)ethoxy, (N,N,N-trimethylammonium)ethoxy, methoxy, ethoxy, methoxymethyl, ethoxymethyl, difluoromethoxy, trifluoromethoxy, and 2,2,2-trifluoroethoxy, and the other groups are as provided in the general Formula (I) above, or as in the first through the fifty-first embodiments.

In a fifty-third embodiment, each R^{10c} is independently selected from fluoro, carboxy, carboxymethyl, (carboxymethyl)oxy, aminocarbonyl, amino, aminomethyl, —SO₂OH, —SO₂NH₂, hydroxy, and aminocarbonylamino, and the other groups are as provided in the general Formula (I) above, or as in the first through the fifty-second embodiments.

In a fifty-fourth embodiment of the invention, R^{11a} is selected from hydrogen, hydroxy, methyl, ethyl, methoxy, and ethoxy, and the other groups are as provided in the general Formula (I) above, or as in the first through the fifty-third embodiments.

In a fifty-fifth embodiment of the invention, R^{11a} is selected from hydrogen, and hydroxy, and the other groups are as provided in the general Formula (I) above, or as in the first through the fifty-fourth embodiments.

In a fifty-sixth embodiment, R^{11b} is selected from (H₂N—C(═NH)—NH)methyl, (H₂N—C(═NH)—NH)ethyl, (H₂N—C(═NH)—NH)propyl, (H₂N—C(═NH)—NH)butyl, phenyl, benzyl, pyridinyl, pyridinylmethyl, indolyl, indolylmethyl, pyridazinyl, pyridazinylmethyl, pyrimidyl, pyrimidylmethyl, pyrazinyl, pyrazinylmethyl, imidazolyl, imidazolylmethyl, pyrazolyl, pyrazolylmethyl, [1,2,4]triazolo[1,5-a]pyridine, oxazolyl, oxazolylmethyl, thiazolyl, and thiazolylmethyl, and the other groups are as provided in the general Formula (I) above, or as in the first through the fifty-fifth embodiments.

In a fifty-seventh embodiment, R^{11b} is selected from phenyl, (H₂N—C(═NH)—NH)ethyl, pyridinyl, indolyl, and imidazolyl, and the other groups are as provided in the general Formula (I) above, or as in the first through the fifty-sixth embodiments.

In a fifty-eighth embodiment of the invention, each R^{11c} is independently selected from fluoro, chloro, bromo, iodo, (carboxymethyl)oxy, (carboxymethyl)oxymethyl, (carboxyethyl)oxy, hydroxy, hydroxymethyl, hydroxyethyl, methoxy, ethoxy, difluoromethoxy, trifluoromethoxy, carboxy, carboxymethyl, aminocarbonyl, aminocarbonylmethyl, (N-methylamino)carbonyl, (N-methylamino)carbonylmethyl, (N,N-dimethylamino)carbonyl, (N,N-dimethylamino)carbonylmethyl, amino, N-methylamino, N,N-dimethylamino, N,N-diethylamino, aminomethyl, (N,N-dimethylamino)methyl, (N,N-diethylamino)methyl, N,N,N-trimethylammonium, N,N,N-trimethylmeth-1-yl-ammonium, N,N,N-triethylammonium, N,N,N-triethylmeth-1-yl-ammonium, methyl, ethyl, isopropyl, cyclopropyl, cyclobutyl, difluoromethyl, trifluoromethyl, aminoethoxy, aminopropoxy, aminoethoxymethyl, (N,N-dimethylamino)ethoxy, N,N,N-trimethyleth-1-yloxy-ammonium, cyano, and methylcarbonylpiperazyl [(N-acetyl)piperazyl], and the other groups are as provided in the general Formula (I) above, or as in the first through the fifty-seventh embodiments.

In a fifty-ninth embodiment of the invention, each R^{11c} is independently selected from (carboxymethyl)oxy, fluoro, chloro, hydroxy, methoxy, trifluoromethoxy, carboxy, carboxymethyl, aminocarbonyl, aminocarbonylmethyl, aminomethyl, aminoethoxy, (N,N-dimethylamino)ethoxy, and methylcarbonylpiperazyl

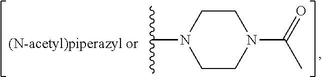

and the other groups are as provided in the general Formula (I) above, or as in the first through the fifty-eighth embodiments.

In a sixtieth embodiment of the invention, R^{12a} is selected from hydrogen, methyl, ethyl, propyl, cyclopropyl, and isopropyl, and the other groups are as provided in the general Formula (I) above, or as in the first through the fifty-ninth embodiments.

In a sixty-first embodiment of the invention, R^{12a} is selected from hydrogen, methyl, ethyl, and n-propyl, and the other groups are as provided in the general Formula (I) above, or as in the first through the sixtieth embodiments.

In a sixty-second embodiment, R^{12b} is selected from hydrogen, methyl, ethyl, and propyl, and the other groups are as provided in the general Formula (I) above, or as in the first through the sixty-first embodiments.

In a sixty-third embodiment, R^{12b} is selected from hydrogen, and methyl, and the other groups are as provided in the general Formula (I) above, or as in the first through the sixty-second embodiments.

In a sixty-forth embodiment of the invention, R^{12a} and R^{12b}, together with the atoms to which the are attached form a saturated ring selected from

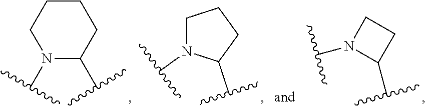 [SMILES: *C1CCCCN1* ; *C1CCCN1* ; *C1CCN1*]

and the other groups are as provided in the general Formula (I) above, or as in the first through the sixty-third embodiments.

In a sixty-fifth embodiment of the invention, R^{12a} and R^{12b}, together with the atoms to which they are attached form

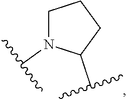 [SMILES: *C1CCCN1*]

and the other groups are as provided in the general Formula (I) above, or as in the first through the sixty-forth embodiments.

In a sixty-sixth embodiment, R^{13a} is hydrogen, methyl, or propyl, and the other groups are as provided in the general Formula (I) above, or as in the first through the sixty-fifth embodiments.

In a sixty-seventh embodiment, R^{13b} is hydrogen or methyl, and the other groups are as provided in the general Formula (I) above, or as in the first through the sixty-sixth embodiments.

In a sixty-eighth embodiment, R^{13c} is selected from indolyl, pyrrolo[2,3-b]pyridinyl, quinolinyl, indazolyl, and naphthyl, and the other groups are as provided in the general Formula (I) above, or as in the first through the sixty-seventh embodiments.

In a sixty-ninth embodiment, R^{13c} is selected from indolyl, and pyrrolo[2,3-b]pyridinyl, and the other groups are as provided in the general Formula (I) above, or as in the first through the sixty-eighth embodiments.

In a seventieth embodiment, each R^{13d} independently is selected from fluoro, chloro, bromo, iodo, methyl, ethyl, propyl, isopropyl, carboxy, carboxymethyl, methoxy, difluoromethoxy, trifluoromethoxy, and ethoxy, and the other groups are as provided in the general Formula (I) above, or as in the first through the sixty-ninth embodiments.

In a seventy-first embodiment, each R^{13d} independently is selected from methyl, chloro, and methoxy, and the other groups are as provided in the general Formula (I) above, or as in the first through the seventieth embodiments.

In a seventy-second embodiment, each R^{14a} independently is selected from amino, hydroxy, N-methylamino, N,N-dimethylamino, N-ethylamino, methoxy, and ethoxy, and the other groups are as provided in the general Formula (I) above, or as in the first through the seventy-first embodiments.

In a seventy-third embodiment, each R^{14a} independently is amino, and the other groups are as provided in the general Formula (I) above, or as in the first through the seventy-second embodiments.

In a seventy-fourth embodiment, each R^{14b} independently is selected from hydrogen, methyl, ethyl, propyl, isopropyl, n-butyl, sec-butyl, isobutyl, n-pentyl, isopentyl, neopentyl, trifluoromethyl, trifluoroethyl, phenyl, benzyl, chlorophenyl, dichlorophenyl, fluorophenyl, difluorophenyl, bromophenyl, iodophenyl, chlorobenzyl, dichlorobenzyl, fluorobenzyl, difluorobenzyl, bromobenzyl, iodobenzyl, naphthyl, naphthylmethyl, pyrazolyl, pyrazolylmethyl, indolyl, indolylmethyl, imidazolyl, imidazolylmethyl, pyridyl, and pyridylmethyl, and the other groups are as provided in the general Formula (I) above, or as in the first through the seventy-third embodiments.

In a seventy-fifth embodiment, each R^{14b} independently is selected from hydrogen, methyl, ethyl, and phenyl, and the other groups are as provided in the general Formula (I) above, or as in the first through the seventy-fourth embodiments.

In a seventy-sixth embodiment, the compound of Formula (1) or a pharmaceutically acceptable salt thereof is:

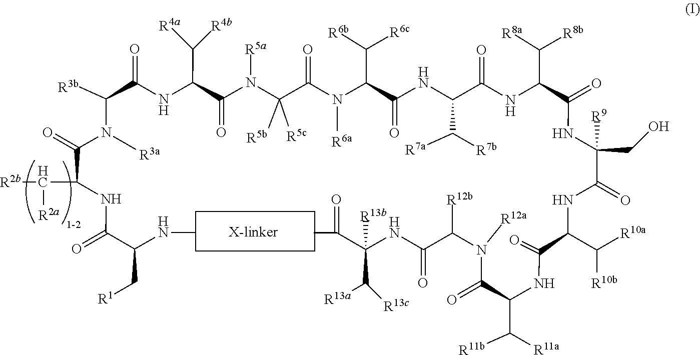

R¹ is selected from hydrogen, C₁₋₁₀ alkyl, (C₁₋₆ alkyl)₀₋₂ amino(C₀₋₁₀ alkyl), (C₁₋₆ alkyl)₃N⁺(C₀₋₆ alkyl), (C₁₋₆ alkyl)₀₋₂ aminocarbonyl(C₀₋₁₀ alkyl), aryl(C₀₋₁₀ alkyl), heteroaryl(C₀₋₁₀ alkyl), (C₃₋₁₂)cycloalkyl(C₀₋₁₀ alkyl), heterocycloalkyl(C₀₋₁₀ alkyl), C₁₋₁₀ fluoroalkyl, C₂₋₁₀ alkenyl, (C₀₋₆ alkyl)carbonylamino(C₀₋₆ alkyl), (C₀₋₆ alkyl)aminocarbonyl(C₀₋₆ alkyl), (C₁₋₆ alkyl)₀₋₂ aminocarbonylamino(C₀₋₆ alkyl), arylcarbonylamino(C₀₋₆ alkyl), arylaminocarbonyl(C₀₋₆ alkyl), heteroarylcarbonylamino(C₀₋₆ alkyl), heteroarylaminocarbonyl(C₀₋₆ alkyl), C₁₋₆ alkyloxy, (C₁₋₆ alkyl)oxy(C₀₋₆ alkyl), ((C₃₋₁₂)cycloalkyl)oxy(C₀₋₆ alkyl), ((C₃₋₁₂)cycloalkyl C₀₋₆ alkyl)oxy(C₀₋₆ alkyl), (C₀₋₆ alkyl)carboxy(C₀₋₆ alkyl), N⁻═N⁺=N—(C₀₋₆ alkyl), and H₂N—C(═NH)NH—(C₀₋₆ alkyl), wherein R¹ is substituted by 0, 1, 2, 3, or 4 Ria substituents each independently selected from C₁₋₆ alkyl, amino, cyano, halo, hydroxy, (C₃₋₁₂)cycloalkyloxy, and C₁₋₆ alkyloxy;
each R^{2a} is independently selected from hydrogen, hydroxy, C₁₋₄ alkyl, fluoro, and C₁₋₄ alkyloxy;
R^{2b} is selected from aryl, heteroaryl, cycloalkyl, and heterocycloalkyl, wherein said heteroaryl contains at least one nitrogen atom,
wherein R^{2b} is substituted by 0, 1, or 2 R^{2c} independently selected from C₁₋₆ alkyl, amino(C₀-6 alkyl), (C₁₋₆ alkyl)₀₋₂ amino(C₀₋₆ alkyl), (C₁₋₆ alkyl)₃N⁺(C₀₋₆ alkyl), aminocarbonyl(C₀₋₆ alkyl), (C₁₋₆ alkyl)₀₋₂ aminocarbonyl(C₀₋₆ alkyl), hydroxy, halo, (C₁₋₆ alkyl)₀₋₂ amino(C₀₋₆ alkyloxy), (C₁₋₆ alkyl)₃N⁺(C₀₋₆ alkyloxy), (C₁₋₆ alkyloxy)carbonyl(C₀₋₆ alkyl), carboxy(C₀₋₆ alkyl), carboxy(C₁₋₆ alkyl)oxy(C₀₋₆ alkyl), C₁₋₆ haloalkyl, C₁₋₆ haloalkyloxy, and C₁₋₆ alkyloxy;
R^{3a} is selected from hydrogen, hydroxy, hydroxy(C₁₋₆ alkyl), amino, amino(C₁₋₆ alkyl), C₁₋₁₀ alkyl, (C₃₋₁₂)cycloalkyl(C₀₋₁₀ alkyl), and carboxy(C₁₋₆ alkyl), wherein R^{3a} may be substituted by 0, 1, or 2 R^{3c} substituents;
R^{3b} is selected from hydrogen, C₁₋₁₀ alkyl, amino(C₁₋₁₀ alkyl), (C₁₋₆ alkyl)₀₋₂ amino(C₁₋₁₀ alkyl), (C₁₋₆ alkyl)₃N⁺(C₁₋₆ alkyl), C₁₋₆ haloalkyl, aryl(C₀₋₁₀ alkyl), heteroaryl(C₀₋₁₀ alkyl), (C₃₋₁₂)cycloalkyl(C₀₋₁₀ alkyl), heterocycloalkyl(C₀₋₁₀ alkyl), (C₁₋₆ alkyl)oxy(C₁₋₆ alkyl), (C₃₋₁₂)cycloalkyloxy(C₁₋₆ alkyl), carboxy(C₁₋₆ alkyl), aminocarbonyl(C₁₋₆ alkyl), (C₁₋₆ alkyl)₀₋₂ aminocarbonyl(C₁₋₆ alkyl), aminocarbonylamino(C₁₋₆ alkyl), (C₁₋₆ alkyl)₀₋₂ aminocarbonylamino(C₁₋₆ alkyl), (C₁₋₆ alkyl)thio(C₁₋₆ alkyl), (C₁₋₆ alkyl)SO₂(C₁₋₆ alkyl), and (C₁₋₆ alkyl)sulfinyl(C₁₋₆ alkyl), wherein R^{3b} may be substituted by 0, 1, or 2 R^{3c} substituents;
each R^{3c} is independently selected from halo, C₁₋₆ alkyl, amino, (C₁₋₆ alkyl)₀₋₂ amino, (C₁₋₆ alkyl)₃N⁺—, (C₁₋₆ alkyl)SO₂(C₀₋₆ alkyl), cyano, cyano(C₁₋₆ alkyl), hydroxy, hydroxy(C₁₋₆ alkyl), (C₁₋₆ alkyl)oxy(C₀₋₆ alkyl), and (C₀₋₆) carboxy(C₀₋₆ alkyl);
and wherein R^{3a} and R^{3b}, together with the atoms to which they are attached, may form a saturated ring system, wherein said saturated ring system may be substituted by 0, 1, or 2, R^{3c} substituents;
R^{4a} is selected from hydrogen, C₁₋₆ alkyl, hydroxy, C₁₋₄ alkyloxy, and fluoro;
R^{4b} is selected from bicyclic heteroaryl(C₀₋₃ alkyl), and bicyclic aryl(C₀₋₃ alkyl),
wherein R^{4b} is substituted with 0, 1, or 2 R^{4c} substituents each independently selected from halo, hydroxy, cyano, nitro, carboxy(C₀₋₆ alkyl), (C₁₋₆ alkyloxy)carbonyl(C₀₋₆ alkyl), C₁₋₆ alkyloxy, (C₁₋₆ alkyl)oxy(C₁₋₆ alkyl), C₁₋₆ alkyl, and C₁₋₆ haloalkyl;
R^{5a} is selected from hydrogen, C₁₋₁₀ alkyl, carboxy(C₁₋₁₀ alkyl), hydroxy, hydroxy(C₁₋₁₀ alkyl), cyano(C₁₋₁₀ alkyl), heterocycloalkyl(C₀₋₁₀ alkyl), (C₁₋₆ alkyl)₀₋₂ aminocarbonyl(C₁₋₆ alkyl), (carboxy(C₁₋₁₀ alkyl))oxy(C₁₋₆ alkyl), aryl(C₀₋₆ alkyl), heteroaryl(C₀₋₆ alkyl), (C₁₋₆ alkyl)oxy(C₁₋₆ alkyl), (C₁₋₆ alkyloxy)carbonyl(C₀₋₆ alkyl), amino(C₀₋₆ alkyl), (C₁₋₆ alkyl)₀₋₂ amino(C₀₋₆ alkyl), (C₁₋₆ alkyl)₃N⁺(C₂₋₁₀ alkyl), (C₃₋₁₂)cycloalkyl(C₀₋₆ alkyl), and C₁₋₁₀ fluoroalkyl, and wherein R^{5a} is substituted by 0, 1, or 2 R^{5d} substituents;
R^{5b} is selected from hydrogen, C₁₋₁₀ alkyl, hydroxy(C₁₋₁₀ alkyl), carboxy(C₁₋₆ alkyl), (C₁₋₆ alkyl)oxy(C₁₋₆ alkyl), aminocarbonyl(C₁₋₆ alkyl), (C₁₋₆ alkyl)₀₋₂ aminocarbonyl(C₁₋₆ alkyl), carboxy(C₁₋₁₀ alkyl)oxy(C₁₋₆ alkyl), cyano(C₁₋₁₀ alkyl), amino(C₁₋₆ alkyl), (C₁₋₆ alkyl)₀₋₂ amino(C₁₋₆ alkyl), (C₁₋₆ alkyl)₃N⁺(C₁₋₆ alkyl), heterocycloalkyl(C₀₋₁₀ alkyl), (C₃₋₁₂)cycloalkyl(C₀₋₁₀ alkyl), and C₁₋₁₀ haloalkyl, wherein R^{5b} is substituted by 0, 1, or 2 R^{5d} substituents, wherein R^{5a} and R^{5b}, together with the atoms to which they are attached, form a saturated mono- or bi-cyclic ring, wherein said mono- or bi-cyclic ring is substituted with 0, 1, or 2 R^{5d};
each R^{5d} is independently selected from halo, hydroxy, C₁₋₁₀ alkyl, hydroxy(C₁₋₁₀ alkyl), carboxy(C₁₋₆ alkyl), C₁₋₆ alkyloxy, (C₁₋₆ alkyl)oxy(C₁₋₆ alkyl), (C₁₋₆ alkyl)₀₋₂ amino(C₀₋₆ alkyl), (carboxy(C₁₋₆ alkyl))oxy(C₁₋₆ alkyl), cyano, and C₁₋₆ haloalkyl, and two R^{5d} substituents together with the atom they are attached to may join together to form a saturated ring;
R^{5c} is hydrogen, C₁₋₄ alkyl, hydroxy(C₁₋₄ alkyl), C₁₋₄ alkyloxy, (C₁₋₄ alkyl)oxy(C₁₋₄ alkyl), hydroxy, amino, or halo;
R^{6a} is selected from hydrogen, hydroxy, amino, C₁₋₄ alkyloxy, and C₁₋₆ alkyl;
R^{6b} is selected from hydrogen, C₁₋₆ alkyl, hydroxy, C₁₋₄ alkyloxy, and fluoro;
R^{6c} is selected from C₁₋₆ alkyloxy, (C₁₋₆ alkyl)oxy(C₁₋₆ alkyl), C₁₋₁₀ haloalkyl, aryl(C₀₋₆ alkyl), heteroaryl(C₀₋₁₀ alkyl), (C₁₋₆ alkyl)SO₂(C₀₋₆ alkyl), hydroxy, hydroxy(C₁₋₆ alkyl), amino(C₀₋₆ alkyl), (C₁₋₆ alkyl)₀₋₂ amino(C₀₋₆ alkyl), (C₁₋₆ alkyl)₀₋₂ aminocarbonyl(C₀₋₆ alkyl), (C₁₋₆ alkyl)carbonylamino(C₀₋₆ alkyl), (C₁₋₆ alkyl)₀₋₂ aminocarbonylamino(C₀₋₆ alkyl), and carboxy(C₀₋₆ alkyl);
R^{7a} is selected from hydrogen, C₁₋₆ alkyl, hydroxy, C₁₋₄ alkyloxy, and fluoro;
R^{7b} is selected from aryl(C₀₋₆ alkyl), heteroaryl(C₀₋₆ alkyl), and (C₃₋₁₂)cycloalkyl(C₀-6 alkyl), wherein R^{7b} is substituted by 0, 1, 2, or 3 R^{7c} substituents each independently selected from C₁₋₆ alkyl, C₁₋₁₀ fluoroalkyl, C₁₋₁₀ fluoroalkyloxy, (C₁₋₆ alkyl)₀₋₂ amino(C₀₋₅ alkyl), (C₁₋₆ alkyl)₃N⁺(C₀₋₅ alkyl), carboxy(C₀₋₆ alkyl), (C₁₋₆ alkyloxy)carbonyl(C₀₋₆ alkyl), carboxy(C₁₋₆ alkyl)oxy(C₀₋₆ alkyl), aminocarbonyl(C₀₋₆ alkyl), (C₁₋₆ alkyl)₀₋₂ aminocarbonyl(C₀₋₆ alkyl), hydroxy, hydroxy(C₁₋₆ alkyl), halo, —(C₀₋₅ alkyl)-(S(═O)₂OH), —(C₀₋₅ alkyl)-(S(═O)₂NH₂), amino(C₀₋₆ alkyl)oxy(C₀₋₆ alkyl), (C₁₋₆ alkyl)₀₋₂ amino(C₀₋₆ alkyl)oxy(C₀₋₆ alkyl), (C₁₋₆ alkyl)₃N⁺(C₀₋₆ alkyl)oxy(C₀₋₆ alkyl), (C₁₋₆ alkyl)₀₋₂ aminocarbonylamino(C₀₋₆ alkyl), C₁₋₁₀ haloalkyl, C₁₋₁₀ haloalkyloxy, and (C₁₋₆ alkyl)oxy(C₀₋₆ alkyl);
R^{8a} is selected from hydrogen, C₁₋₆ alkyl, hydroxy, C₁₋₄ alkyloxy, C₁₋₆ fluoroalkyl, C₁₋₄ fluoroalkyloxy, and halo;
R^{8b} is selected from aryl(C₀₋₃ alkyl) and heteroaryl(C₀₋₃ alkyl), wherein R^{8b} is substituted by 0, 1, or 2 R^{8c} substituents each independently selected from C₁₋₄ alkyl, halo, cyano, nitro, carboxy, amino, hydroxy, C₁₋₆ fluoroalkyl, C₁₋₆ fluoroalkyloxy, C₁₋₆ alkyloxy, amino(C₀₋₃ alkyl), and hydroxy(C₁₋₆ alkyl);
R⁹ is selected from hydrogen, and C₁₋₄ alkyl;
R^{10a} is selected from hydrogen, hydroxy, C₁₋₆ alkyl, C₁₋₄ alkyloxy, and fluoro;
R^{10b} is selected from (C₃₋₁₂)cycloalkyl(C₀₋₃ alkyl), aryl(C₀₋₃ alkyl) and heteroaryl(C₀₋₃ alkyl), wherein said heteroaryl contains 1, 2, or 3 nitrogen atoms, wherein R^{10b} is substituted by 0, 1, or 2 R^{10c} wherein each R^{10c} is independently selected from C₁₋₁₀ alkyl, C₁₋₁₀ fluoroalkyl, C₁₋₁₀ fluoroalkyloxy, amino(C₀₋₆ alkyl), (C₁₋₆ alkyl)₀₋₂ amino(C₀₋₆ alkyl), (C₁₋₆ alkyl)₃N⁺(C₀₋₆ alkyl), hydroxy, cyano, halo, hydroxy(C₁₋₆ alkyl), aminocarbonyl(C₀₋₆ alkyl), (C₁₋₆ alkyl)₀₋₂ aminocarbonyl(C₀₋₆ alkyl), (C₁₋₆ alkyl)₀₋₂ aminocarbonylamino(C₀₋₆ alkyl), (C₁₋₆ alkyl)carbonylamino(C₀₋₆ alkyl), carboxy(C₀₋₆ alkyl), (C₁₋₆ alkoxy)carbonyl(C₀₋₆ alkyl), carboxy(C₁₋₆ alkyl)oxy(C₀₋₆ alkyl), —(C₀₋₅ alkyl)-(S(═O)₂OH), —(C₀₋₅ alkyl)-(S(═O)₂NH₂), amino(C₀₋₆ alkyl)oxy(C₀₋₆ alkyl), (C₁₋₆ alkyl)₀₋₂ amino(C₀₋₆ alkyl)oxy(C₀₋₆ alkyl), (C₁₋₆ alkyl)₃N⁺(C₀₋₆ alkyl)oxy(C₀₋₆ alkyl), (C₁₋₆ alkyl)oxy(C₀₋₆ alkyl), C₁₋₆ haloalkyloxy, C₁₋₆ haloalkyl, (C₃₋₁₂)cycloalkyl(C₀₋₆ alkyl), and heterocycloalkyl(C₀₋₆ alkyl);
R^{11a} is selected from hydrogen, hydroxy, C₁₋₆ alkyl, C₁₋₄ alkyloxy, and fluoro;
R^{11b} is selected from aryl(C₀₋₃ alkyl) and heteroaryl(C₀₋₃ alkyl) wherein said heteroaryl contains 1, 2, or 3 nitrogen atoms, and H₂N—C(═NH)NH—(C₁₋₆ alkyl), wherein R^{11b} is substituted by 0, 1, 2, 3 or 4 R^{11c} substituents each independently selected from C₁₋₆ alkyl, amino(C₀₋₆ alkyl), (C₁₋₆ alkyl)₀₋₂ amino(C₀₋₆ alkyl), (C₁₋₆ alkyl)₃N⁺(C₀₋₆ alkyl), hydroxy, cyano, halo, hydroxy(C₁₋₆ alkyl), (C₁₋₆ alkyl)₀₋₂ aminocarbonyl(C₀₋₆ alkyl), carboxy(C₀₋₆ alkyl), carboxy(C₁₋₆ alkyl)oxy(C₀₋₆ alkyl), amino(C₀₋₆ alkyl)oxy(C₀₋₆ alkyl), (C₁₋₆ alkyl)₀₋₂ amino(C₀₋₆ alkyl)oxy(C₀₋₆ alkyl), (C₁₋₆ alkyl)₃N⁺(C₀₋₆ alkyl)oxy(C₀₋₆ alkyl), (C₁₋₆ alkyl)oxy(C₀₋₆ alkyl), C₁₋₆ haloalkyloxy, C₁₋₆ haloalkyl, (C₃₋₁₂)cycloalkyl(C₀₋₆ alkyl), heterocycloalkyl(C₀₋₆ alkyl), and ((C₁₋₆ alkyloxy)carbonyl)heterocycloalkyl(C₀₋₁₀ alkyl);
R^{12a} is selected from hydrogen, hydroxy, amino, C₁₋₁₀ alkyl, and (C₃₋₁₂)cycloalkyl(C₀₋₆ alkyl);
R^{12b} is selected from hydrogen, C₁₋₁₀ alkyl, and (C₃₋₁₂)cycloalkyl(C₀₋₆ alkyl);
wherein R^{12a} and R^{12b}, together with the atoms to which they are attached, may form a saturated ring;
R^{13a} is selected from hydrogen, and C₁₋₄ alkyl;
R^{13b} is selected from hydrogen, hydroxy, C₁₋₆ alkyl, C₁₋₄ alkyloxy, and fluoro;
R^{13c} is selected from a bicyclic nitrogen-containing heteroaryl having 1 or 2 nitrogen and bicyclic-aryl and wherein R^{13c} is substituted independently by 0, 1, or 2 R^{13d} substituents each independently selected from halo, C₁₋₆ alkyl, carboxy(C₀₋₄ alkyl), C₁₋₄ haloalkyloxy, and C₁₋₄ alkyloxy;

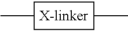

is selected from

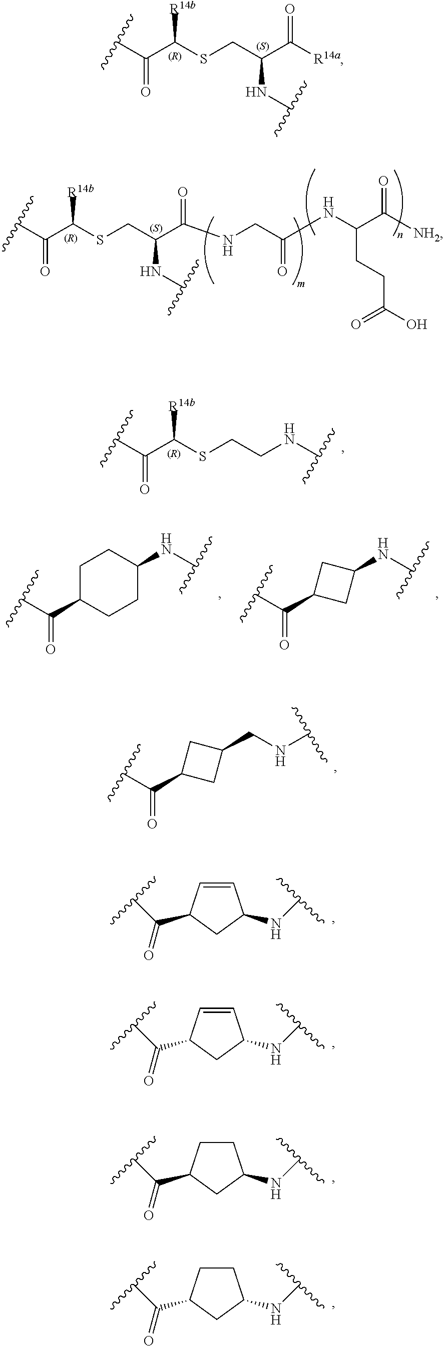

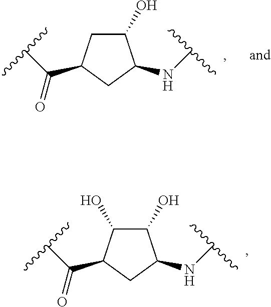

wherein R^{14a} is selected from amino, hydroxy, (C₁₋₆ alkyl)₀₋₂ amino, and C₁₋₆ alkyloxy;
m is selected from 1, 2, 3, or 4;
n is selected from 1, 2, 3, or 4; and
R^{14b} is selected from hydrogen, C₁₋₈ alkyl, aryl(C₀₋₆ alkyl), and heteroaryl(C₀₋₆ alkyl), wherein R^{14b} is substituted by 0, 1, 2, or 3 halo.

In certain embodiments, the present disclosure provides a compound of Formula (I), wherein the compound is selected from the group consisting of SEQ ID NOS: 1-289 as set forth in Table 1.

In specific embodiments, the present disclosure provides a compound of Formula (I), wherein the compound is selected from the group consisting of (SEQ ID NOS 5, 10, 18, 22, 30, 31, 41, 45, 48, 51, 52, 53, 60, 68, 77, 83, 87, 104, 183, 196, 206, 210, 214, 221, 229, 237, 247, 250, 254, 257, and 265 respectively, in order of appearance):

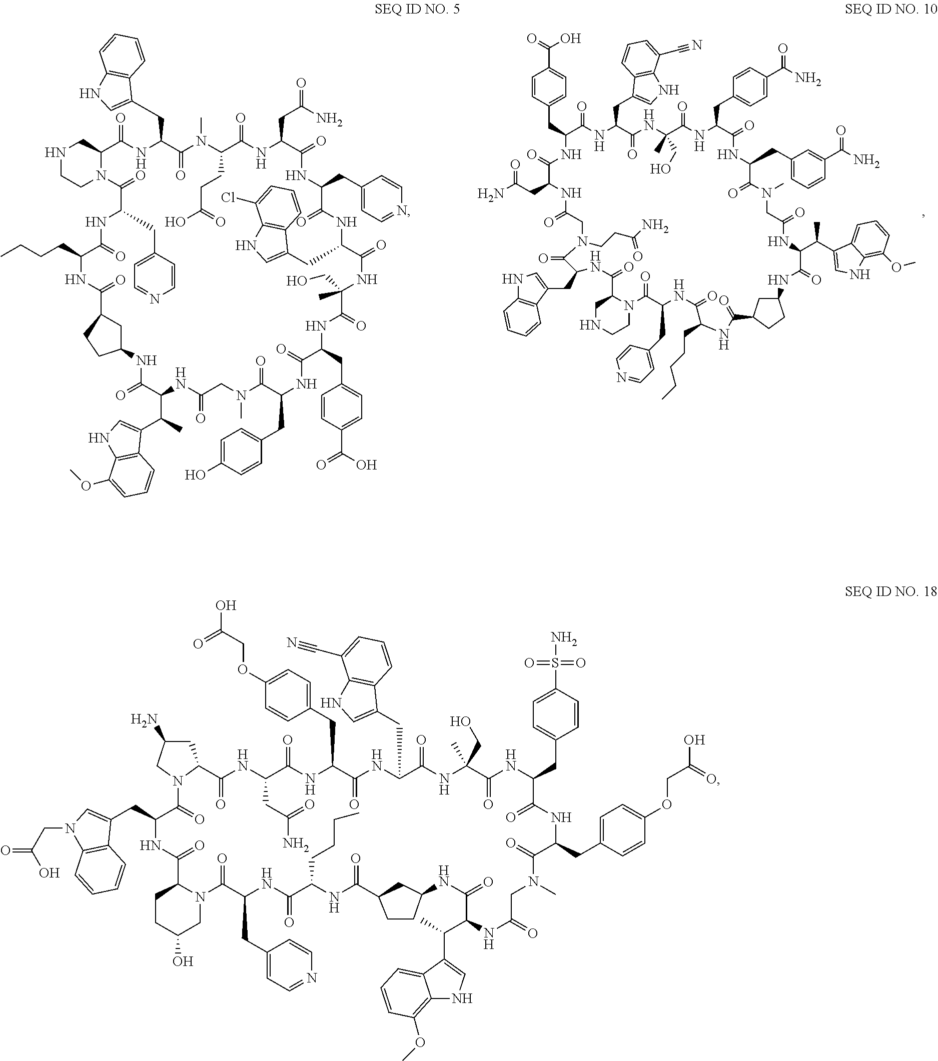

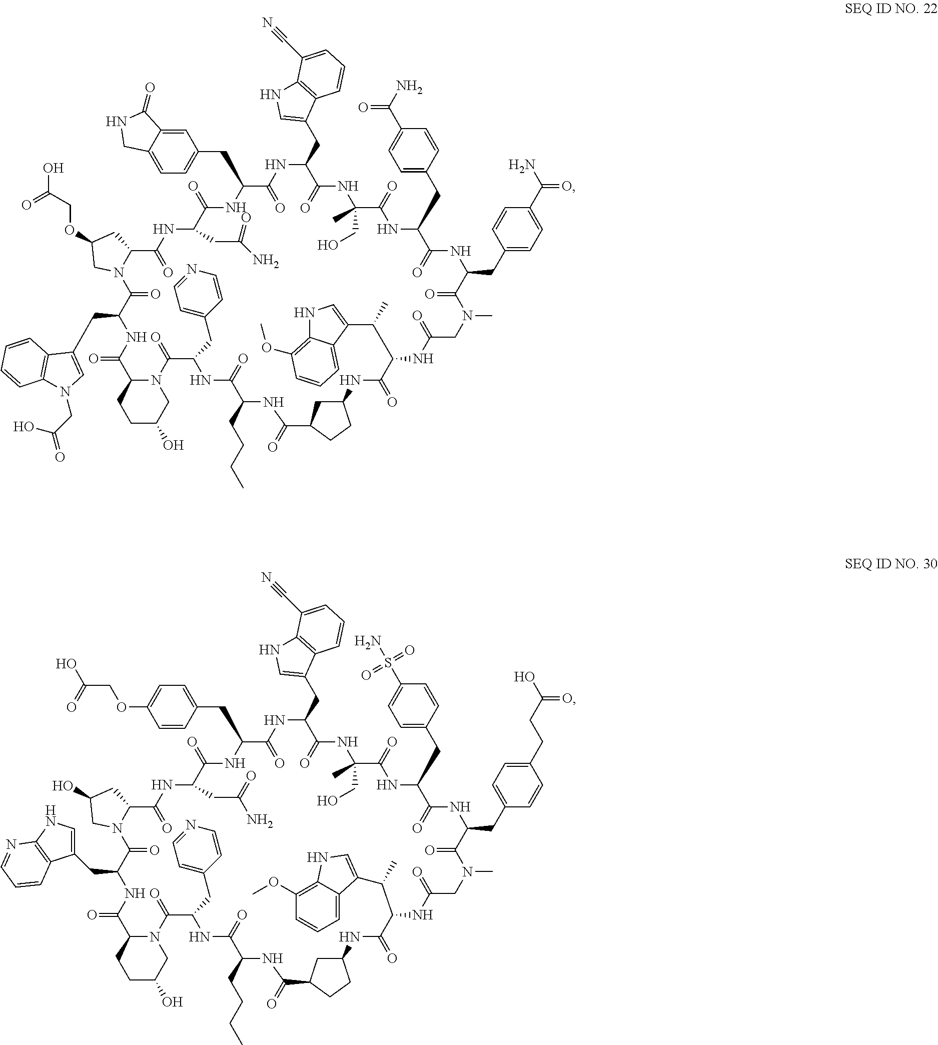

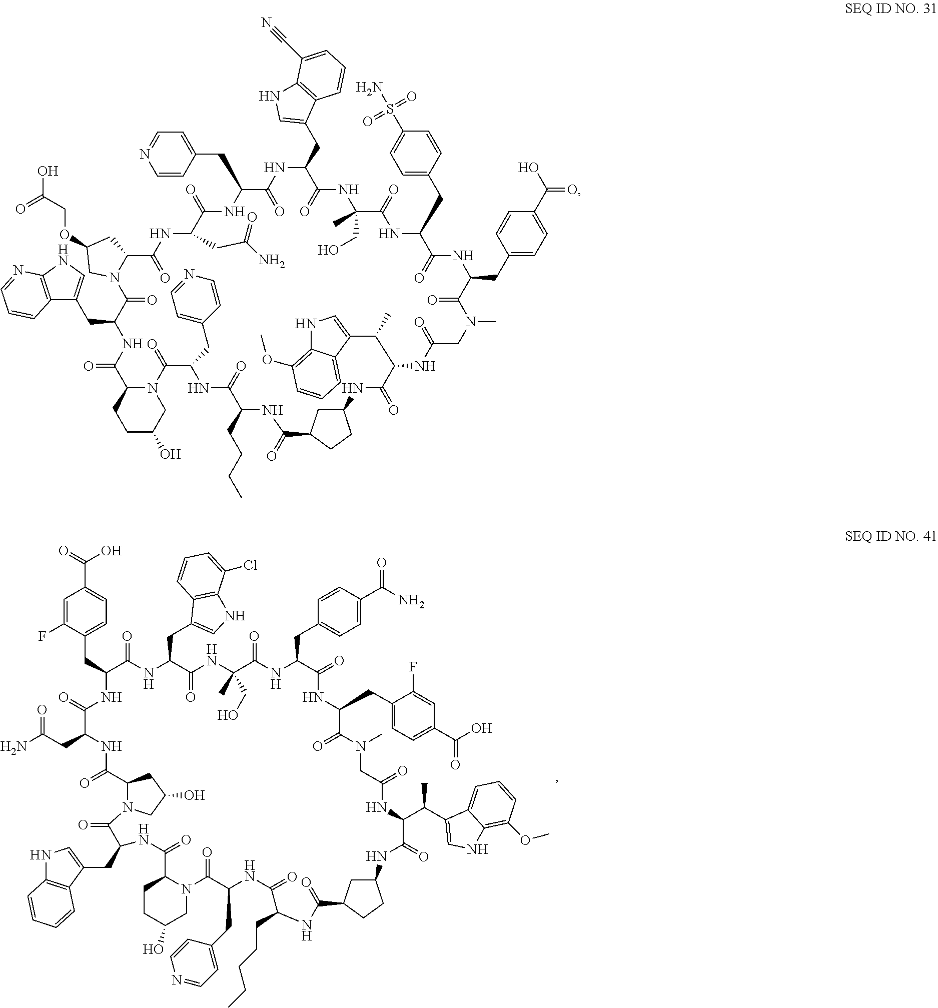

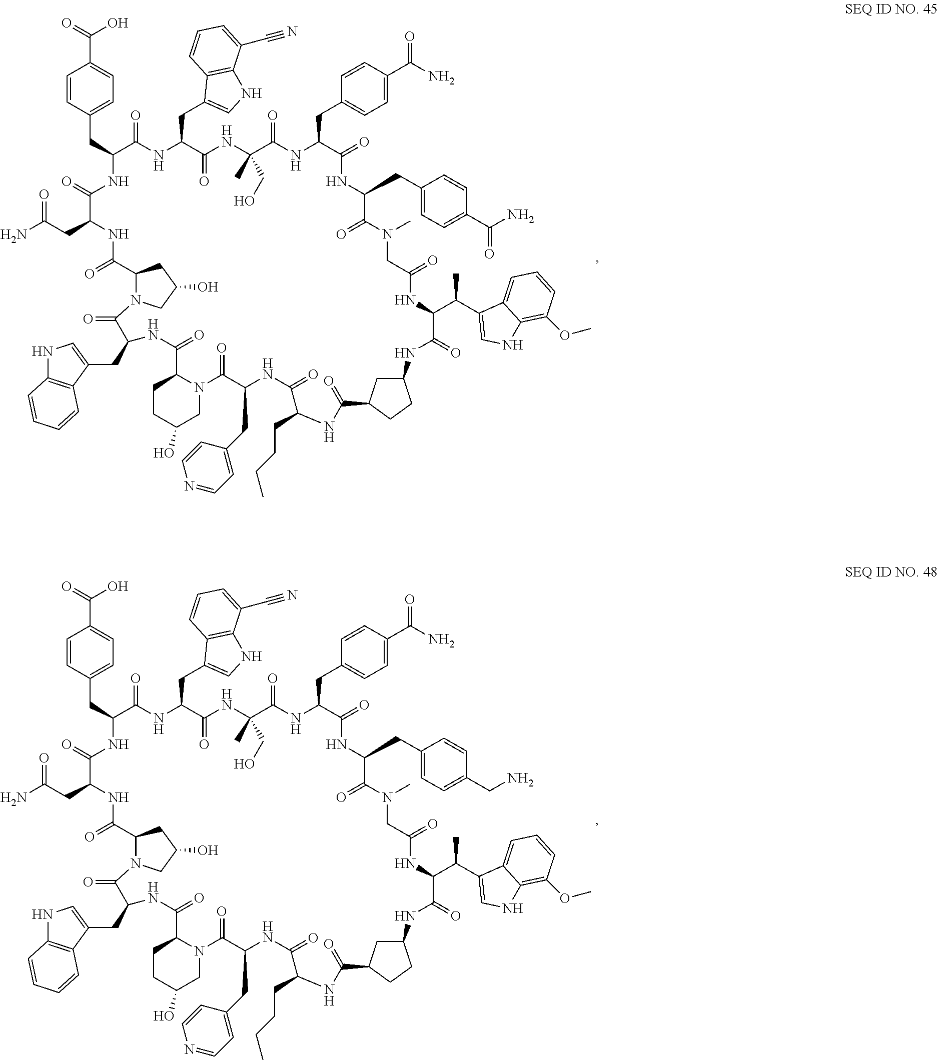

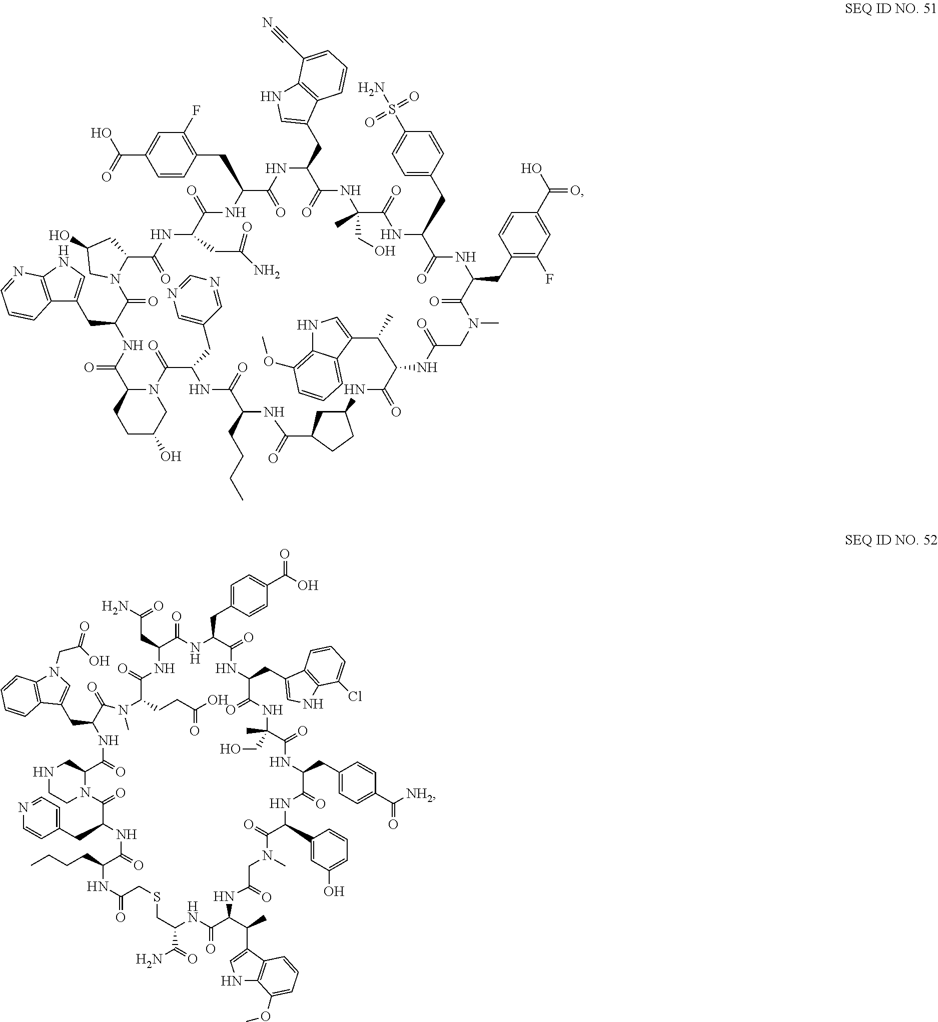

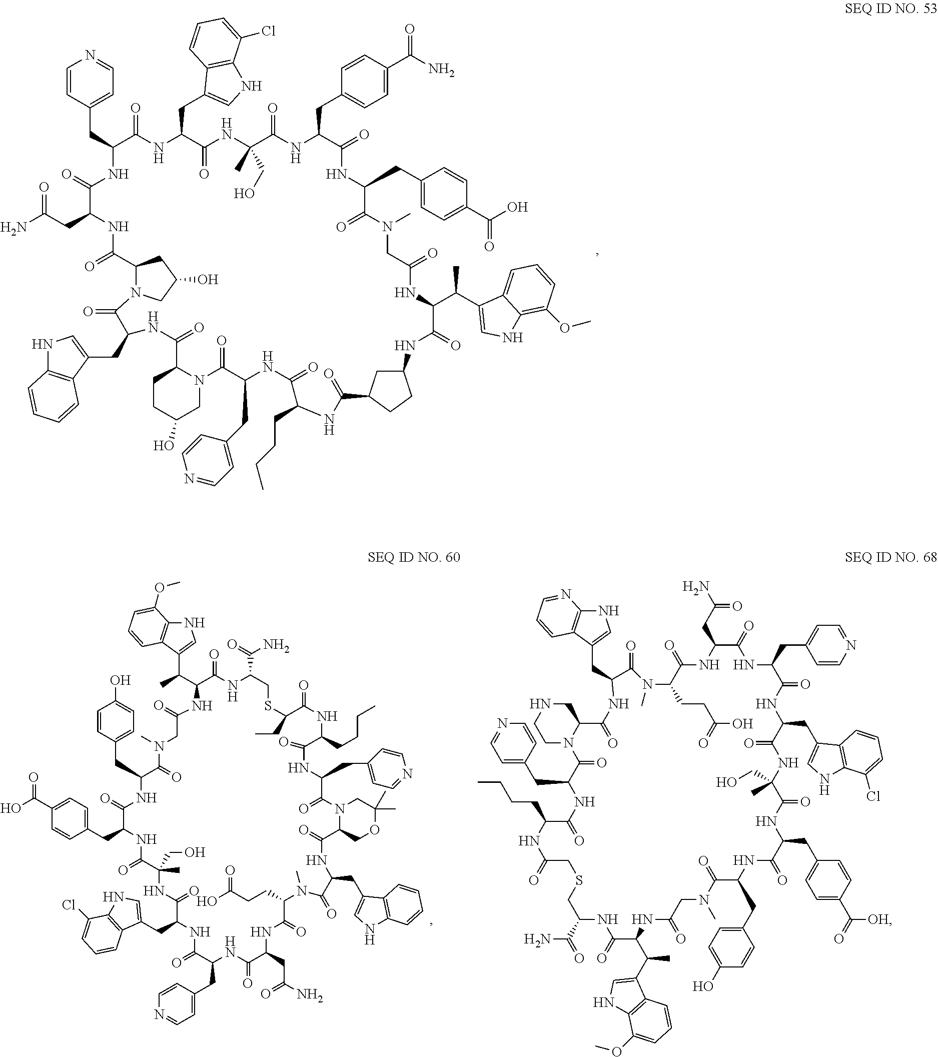

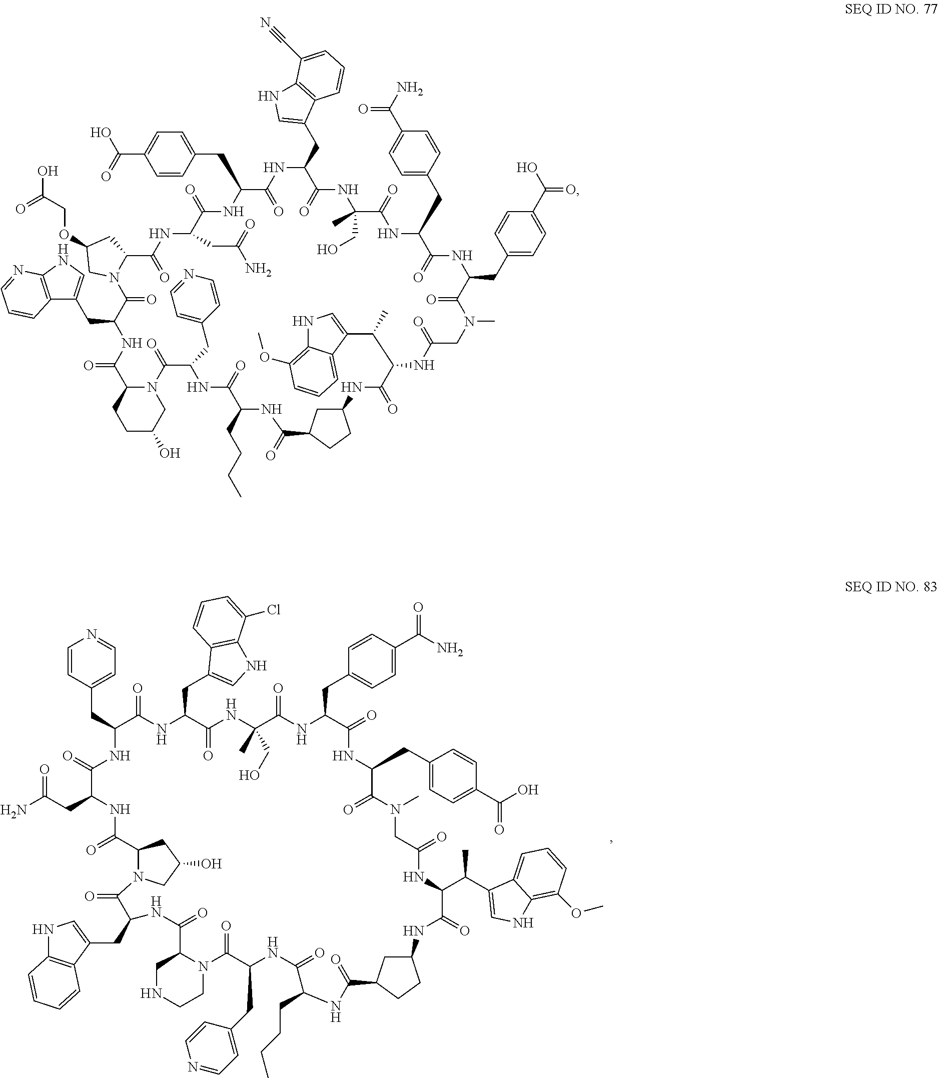

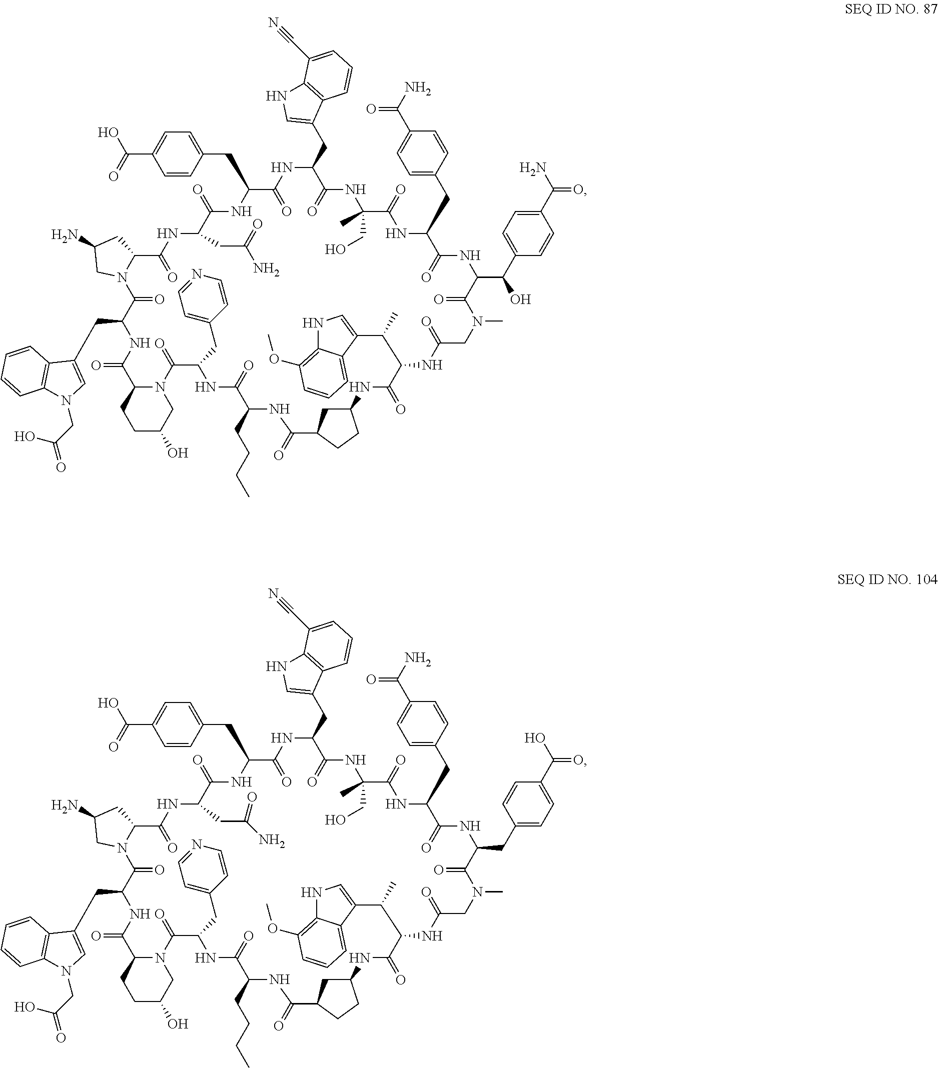

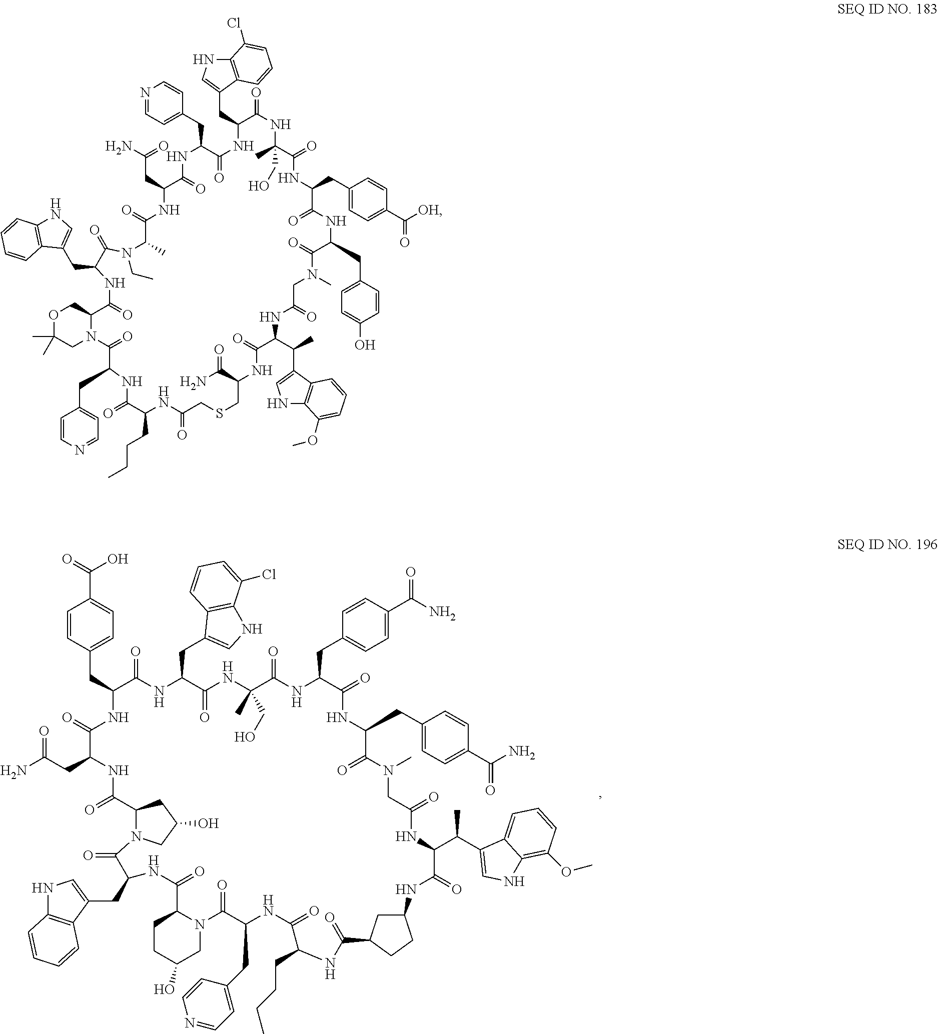

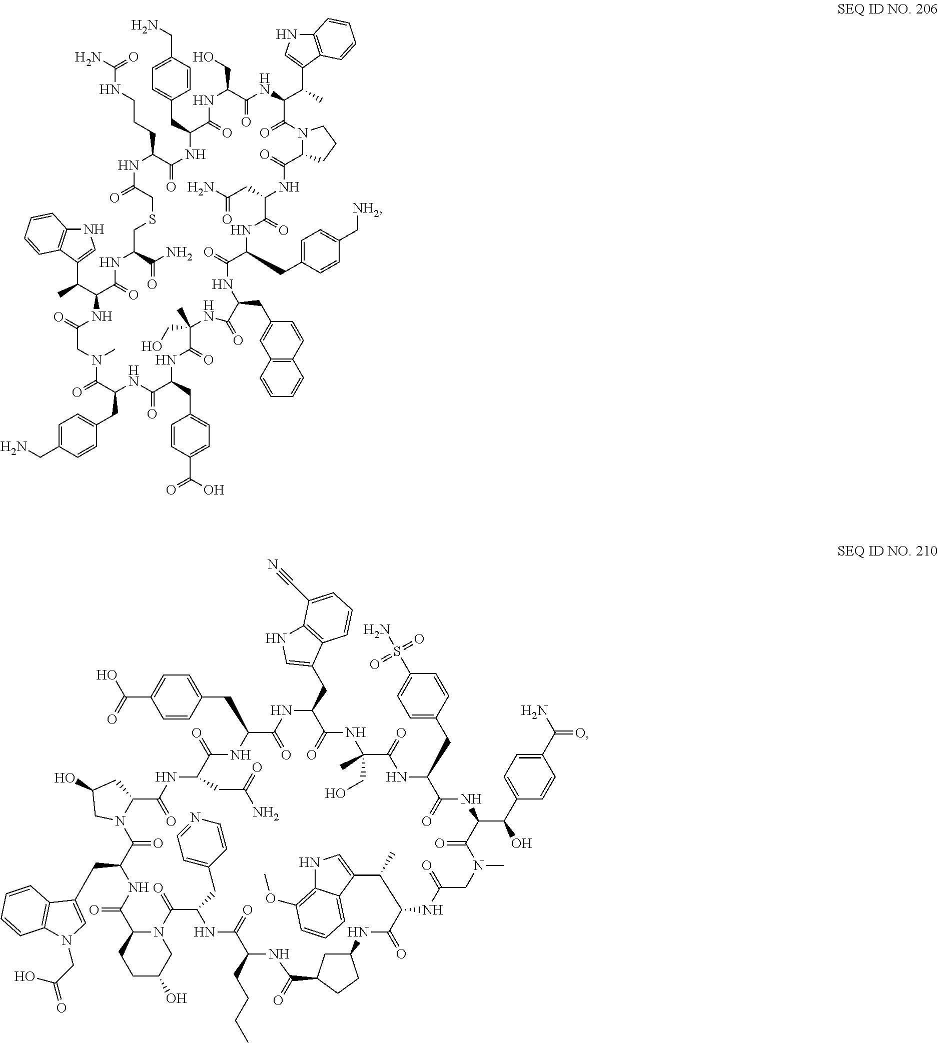

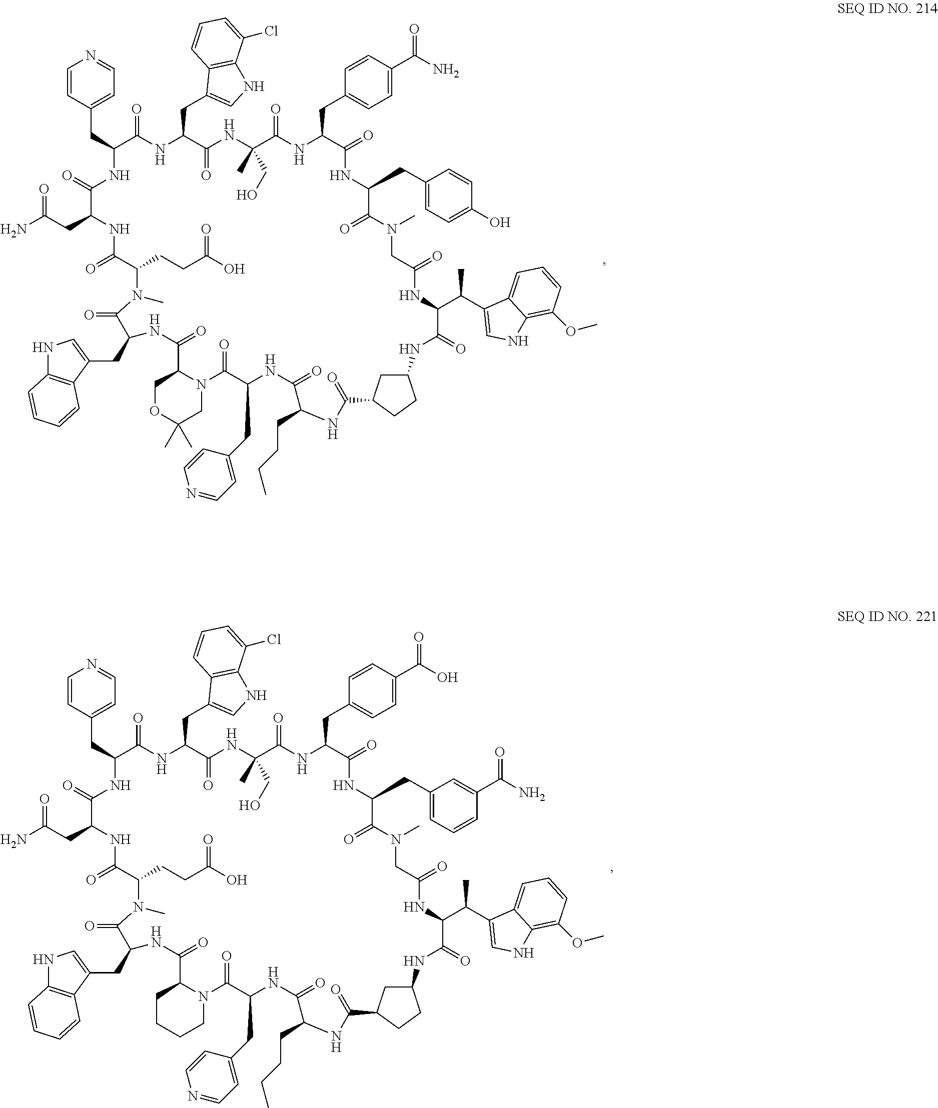

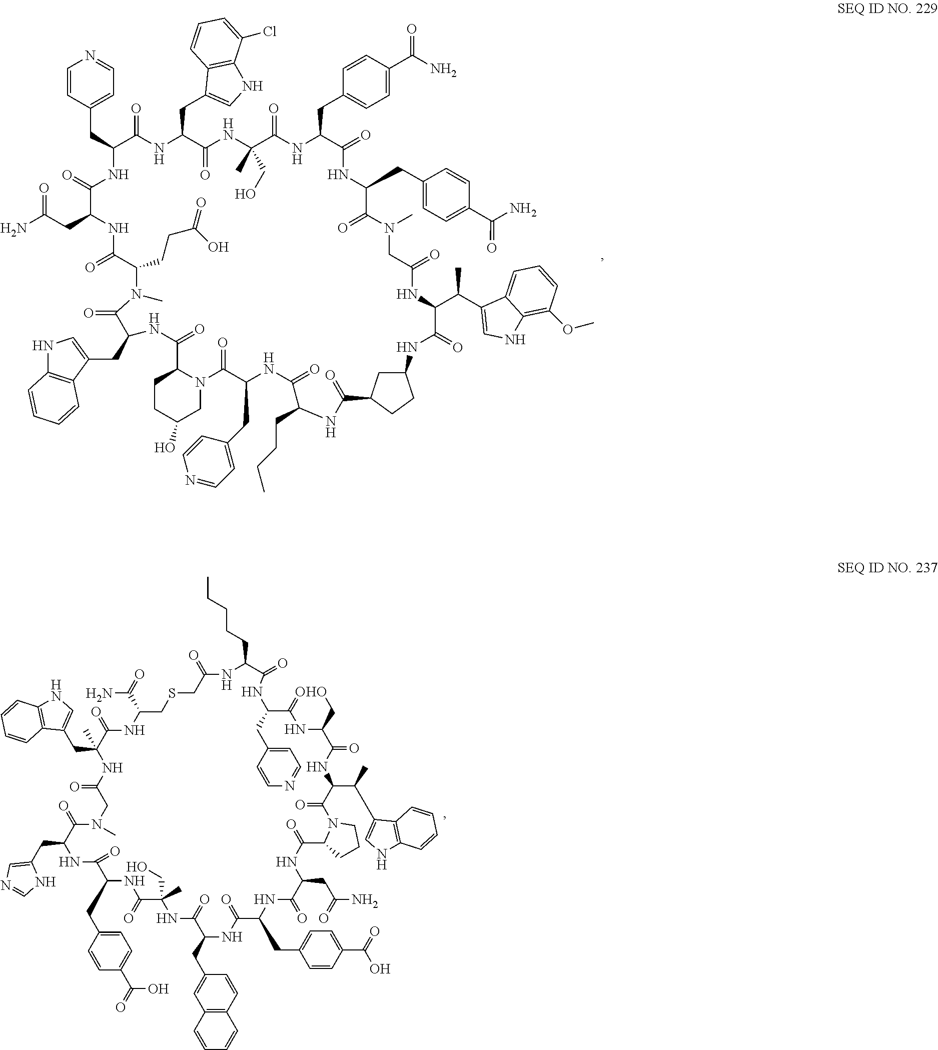

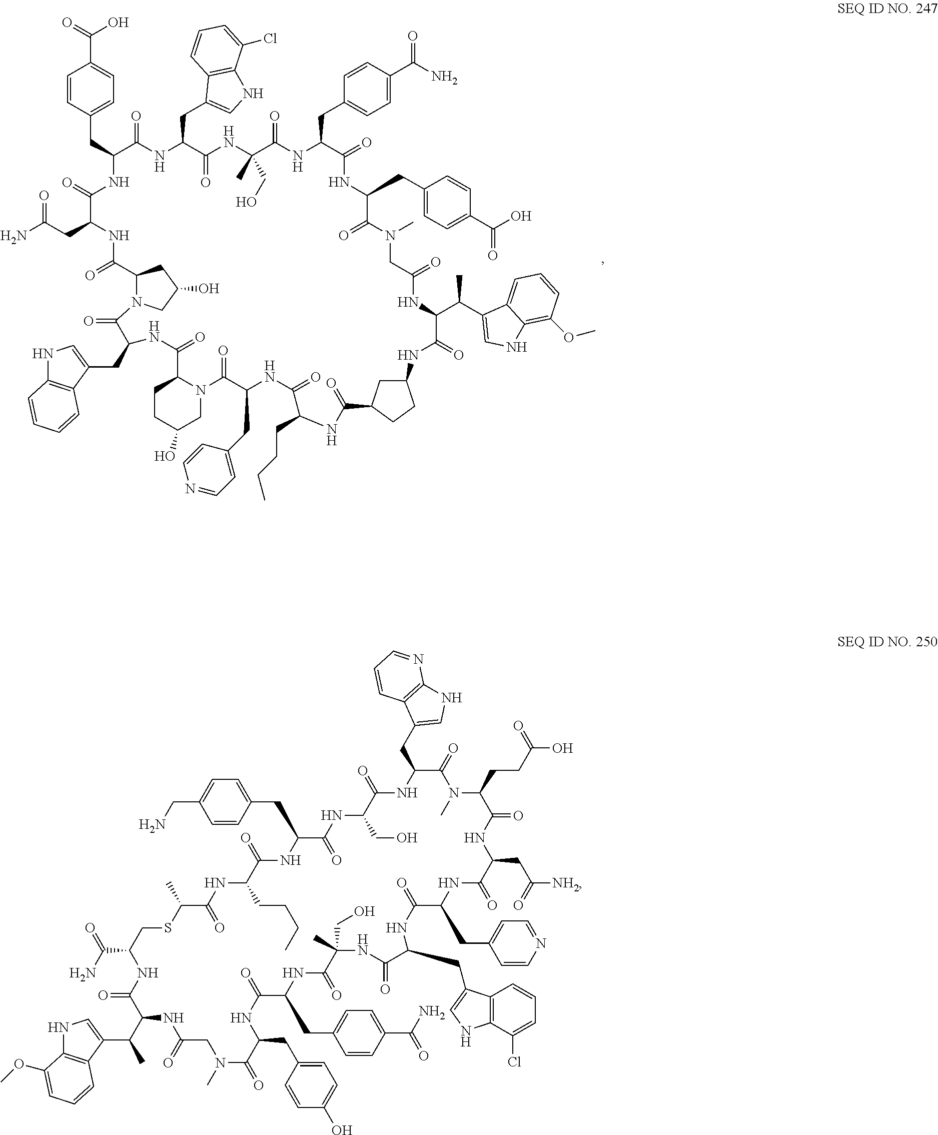

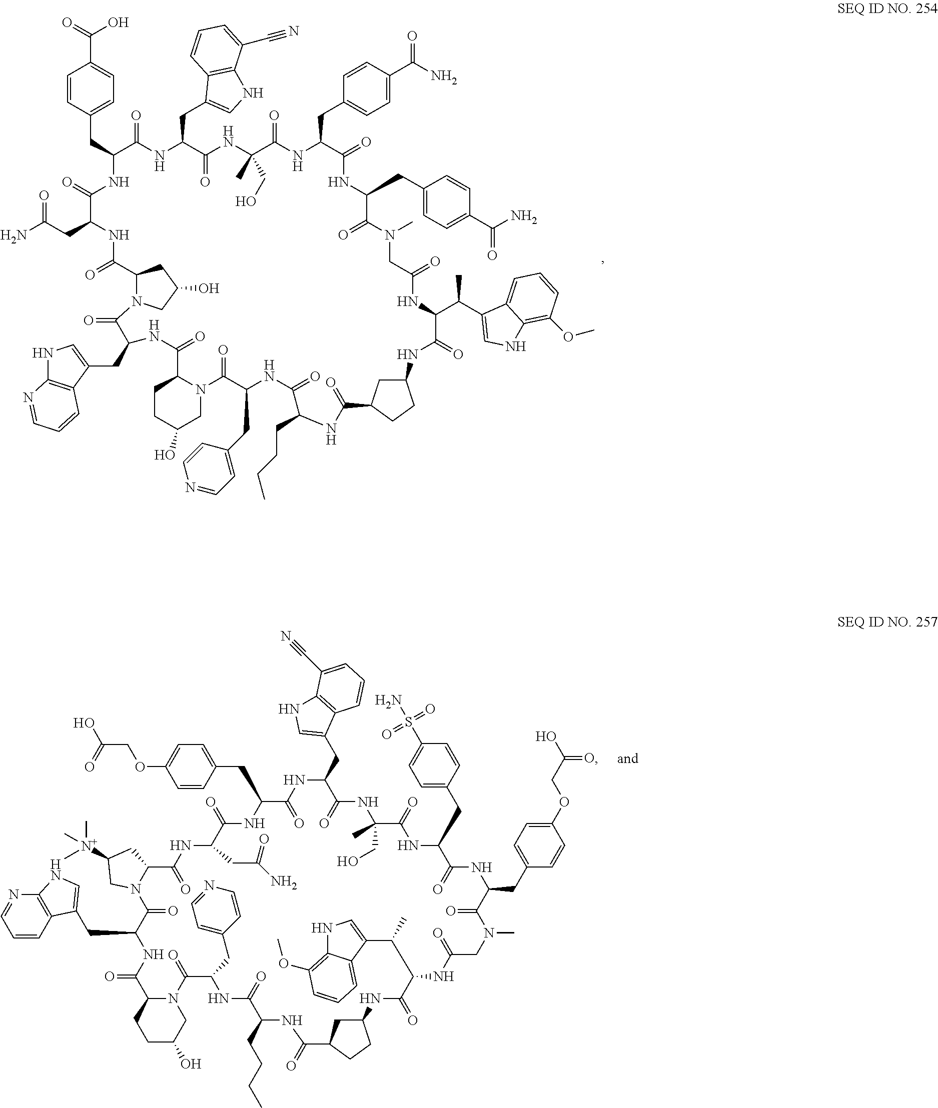

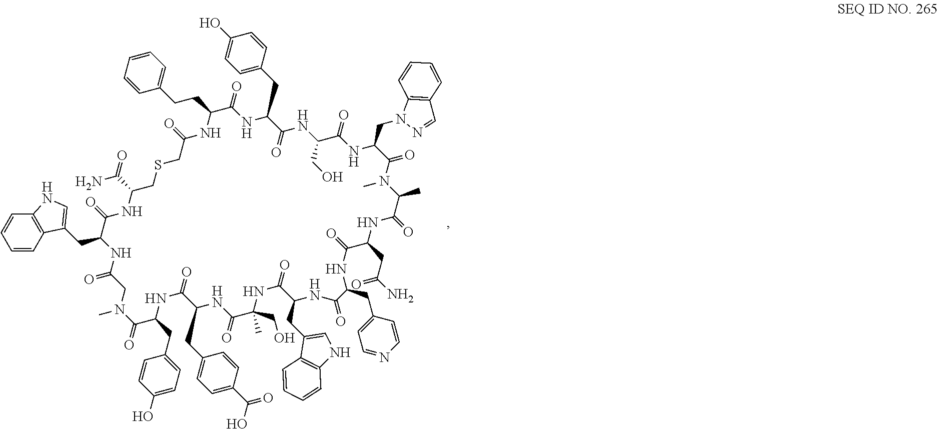 [SMILES: C[C@H]1C(=O)N[C@@H](CC(N)=O)C(=O)N[C@@H](Cc2ccncc2)C(=O)N[C@@H](Cc2c[nH]c3ccccc23)C(=O)N[C@@](C)(CO)C(=O)N[C@@H](Cc2ccc(C(=O)O)cc2)C(=O)N[C@@H](Cc2ccc(O)cc2)C(=O)N(C)CC(=O)N[C@@H](Cc2c[nH]c3ccccc23)C(=O)N[C@H](C(N)=O)CSCC(=O)N[C@@H](CCc2ccccc2)C(=O)N[C@@H](Cc2ccc(O)cc2)C(=O)N[C@@H](CO)C(=O)N[C@@H](Cn2ncc3ccccc32)C(=O)N1C]

or a pharmaceutically acceptable salt thereof.

The mechanism of action (MOA) of anti-TNFα biologics such as infliximab, adalimumab, golimumab and certolizumab, involves binding to the cytokine TNFα and thus, inhibition of engagement with both TNFR1 and TNFR2. While not being bound by any specific theory, the Applicants believe that the compounds of the disclosure selectively inhibit TNFR1 to specifically attenuate the proinflammatory activities of TNFα-mediated TNFR1 signaling, and to spare/passively enable TNFα-TNFR2 pro-homeostatic signaling, which may confer better therapeutic efficacy than the standard of care anti-TNFα biologics.

### Definitions

Unless defined otherwise, all technical and scientific terms used herein have the same meaning as is commonly understood by one of skill in the art to which this invention belongs.

As used throughout this disclosure, “a compound of the disclosure” ˜, “a compound of the present disclosure” and “a compound disclosed herein” are used interchangeably and to be understood to include the disclosed cyclic peptides and compounds of Formula (I). The compounds of Formula (I) can form salts which are also within the scope of the present disclosure. The term “salt(s)”, as employed herein, denotes acidic salts formed with inorganic and/or organic acids, as well as basic salts formed with inorganic and/or organic bases. In addition, when a compound of Formula (I) contains both a basic moiety, such as, but not limited to an amino group, pyrrolidine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the compounds of Formula (I) may be formed, for example, by reacting a compound of Formula (I) with an amount of acid or base, such as an equivalent amount, in a medium such as one in which the salt precipitates or in an aqueous medium followed by lyophilization.

The term “C₀” or “C0” or “C₀” as employed in expressions such as “C₀₋₆ alkyl” and “C₀₋₆ alkyl” means a direct covalent bond; or when the term appears at the terminus of a substituent, C₀₋₆ alkyl means hydrogen or C₁₋₆ alkyl. Similarly, when an integer defining the presence of a certain number of atoms in a group is equal to zero, it means that the atoms adjacent thereto are connected directly by a bond. For example, in the structure

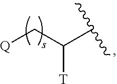

wherein s is an integer equal to zero, 1 or 2, the structure is

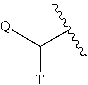

when s is zero.

“Acyl” means a —C(═O)-alkyl group, wherein alkyl is as defined below. The bond to the parent group is through the carbon atom of the carbonyl group.

“Acetyl” means the radical —C(═O)CH₃.

The term “alkyl”, as well as other groups having the prefix “alk”, such as alkoxy, dialkylamino, and trialkylammonium, and the like, refers to an aliphatic hydrocarbon group having one of its hydrogen atoms replaced with a bond. An alkyl group may be straight or branched and contain from about 1 to about 10 carbon atoms. In one embodiment, an alkyl group contains from about 1 to about 10 carbon atoms. In different embodiments, an alkyl group contains from 1 to 6 carbon atoms (C₁₋₆ alkyl) or from about 1 to about 4 carbon atoms (C₁-C₄ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. In one embodiment, an alkyl group is linear. In another embodiment, an alkyl group is branched. Unless otherwise indicated, an alkyl group is unsubstituted.

“Alkenyl” refers to an aliphatic hydrocarbon group containing at least one carbon-carbon double bond and which may be straight or branched and having the indicated number of carbon atoms. Preferably alkenyl contains one carbon to carbon double bond, and up to four nonaromatic carbon-carbon double bonds may be present. Examples of alkenyl groups include ethenyl, propenyl, n-butenyl, 2-methyl-1-butenyl, 3-methylbut-2-enyl, n-pentenyl, octenyl and decenyl.

“Alkoxy” and “alkyloxy” are used interchangeably and refer to an alkyl (carbon and hydrogen chain) group linked to oxygen (R-0). Non-limiting examples of alkoxy are methoxy (CH₃O—), ethoxy (CH₃CH₂O—) and propoxy (CH₃CH₂CH₂O—).

“Amino” means a —NH₂ group, which may be substituted (wherein one or both of the hydrogen atoms are replaced), as further defined herein.

“Amino acid” refers to naturally-occurring α-amino acids and their stereoisomers, as well as unnatural amino acids (such as α,α-disubstituted amino acids, β-substituted amino acids, β-amino acids and substituted amino acids) and their stereoisomers. In the sequences given for the peptides (compounds) according to the present disclosure, the amino acid residues have their conventional meaning. Thus, “G” or “Gly” is glycine, “W” or “Trp” is tryptophan, “A” or “Ala” is alanine, “S” or “Ser” is serine, and so on. It is to be understood that “d” or “D” or “D” isomers are designated by a “D” or “D” before the three-letter code or amino acid abbreviation or amino acid name, such that for example D-Ala or D-Ala or dA is the D isomer of L-alanine. Amino acid residues not encompassed by the foregoing have the definitions provided in the Abbreviation Table in the Examples section below.

“Aminocarbonyl” means —C(═O)NH₂, wherein the amino moiety may be substituted (wherein one or both of the hydrogen atoms are replaced), as further defined herein.

“Aryl” means a monocyclic, bicyclic, or tricyclic carbocyclic aromatic ring or ring system containing 5-14 carbon atoms, wherein at least one of the rings is aromatic. Examples of aryl include phenyl, biphenyl, and naphthyl. In one embodiment of the present invention, aryl is phenyl.

“Azido” means a radical derived from an azide anion having the structure —N═N⁺═N—, such as, for example, 2-azidoethyl

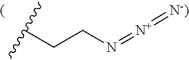

and 3-azidopropyl

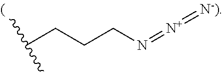

“Bicyclic ring system” refers to two joined rings. The rings may be fused, i.e., share two adjacent atoms, or “spirocyclic”, i.e., share only a single atom.

“Carbonyl” means a functional group composed of a carbon atom double-bonded to an oxygen atom (C═O).

“Carbonylamino” means —NHC(═O)H, wherein the amino moiety may be substituted (wherein one or both of the hydrogen atoms are replaced), as further defined herein.

“Carboxy” means a —CO₂H group. The bond to the parent group is through the carbon atom of the carbonyl component.

“Celite®” (Fluka) diatomite is diatomaceous earth and can be referred to as “celite”.

“Cycloalkyl” or “C₃₋₁₂ cycloalkyl” means any univalent non-aromatic radical derived from a monocyclic, bicyclic, tricyclic, or tetracyclic ring system having 3 to 12 ring carbons atoms. These non-aromatic radicals, which have 3, 4, 5, 6, 7, 8, or up to 12 carbon ring atoms may be fully saturated, or partially unsaturated. Unless stated specifically in the specification, the cycloalkyl radical may be a monocyclic, bicyclic, tricyclic, or tetracyclic ring system, which may include fused or bridged ring systems. Here, the point of attachment for a “cycloalkyl” to the rest of the molecule is on the saturated ring. Bicyclic cycloalkyl ring systems include fused ring systems, where two rings share two atoms (e.g., decalin), spiro ring systems where two rings share one atom (e.g., spiro[4.5]decanyl) and bridged groups (e.g., norbornyl).

Additional examples within the above meaning include, but are not limited to univalent radicals of cyclopropyl, cyclobutyl, cyclopentyl, cyclohexyl, cycloheptyl, bicyclo[2.2.2]octanyl, bicyclo[1.1.1]pentanyl, bicyclo[2.2.1]heptanyl, [1.1.1]-bicyclo pentane, bicyclo[3.1.0]hexanyl, cyclohexenyl, cyclopentenyl, 1-decalinyl, spiro[2.4]heptyl, spiro[2.2]pentyl, and norbornyl.

The term “C₃₋₈ cycloalkyl” (or “C₃-C₈ cycloalkyl” or “C₃₋₈ cycloalkyl”) means a cyclic ring of an alkane having three to eight total carbon atoms (i.e., cyclopropyl, cyclobutyl, cyclopentyl, cyclohexyl, cycloheptyl, or cyclooctyl). The terms “C₃₋₇ cycloalkyl”, “C₃₋₆ cycloalkyl”, “C₅₋₇ cycloalkyl” and the like have analogous meanings.

“Dialkylamino” means an amino (such as N,N-dimethylamino or

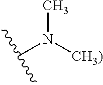

containing two alkyl groups attached to the amino nitrogen.

“Fluoroalkyl” includes mono-substituted as well as multiple fluoro-substituted linear and branched alkyl groups, up to perfluoro substituted alkyl. For example, fluoromethyl, 1,1-difluoroethyl, difluoromethyl, trifluoromethyl or 3,3,4,4,4-pentafluorobutyl are included.

“Guanidino” means a radical containing the univalent group R₂NC(═NR)NH— derived from guanidine where R independently is C₀₋₆ alkyl, such as 3-guanidinopropyl

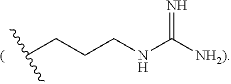

“Halogen” or “halo”, unless otherwise indicated, includes fluorine (fluoro), chlorine (chloro), bromine (bromo) and iodine (iodo). In one embodiment, halo is fluoro (—F) or chloro (—Cl).

“Haloalkyl” refers to an alkyl group as described above wherein one or more (in particular, 1 to 10 hydrogen atoms have been replaced by halogen atoms, with up to complete substitution of all hydrogen atoms with halo groups. C₁₋₆ haloalkyl, for example, includes —CH₂F, —CHF₂, CF₃, —CCl₃, —CF₂CF₃, —CHFCH₃, and the like.

The term “heteroalkyl” refers to an alkyl group where 1, 2, 3, or 4 of the carbon atoms is substituted by a heteroatom independently chosen from nitrogen (N), oxygen (O), or sulfur (S).

The term “heteroaryl”, as used herein, represents a stable monocyclic, bicyclic or tricyclic ring system containing 5 to 14 ring atoms, including at least one ring heteroatom selected from nitrogen (N), sulfur (S) (including S═O and SO₂) and oxygen (O), wherein at least one of the heteroatoms containing rings is aromatic. In the case of a heteroaryl ring system where one or more of the rings are saturated and contain one or more nitrogen (N) atoms, the nitrogen (N) can be in the form of quaternary amine or quaternary ammonium cation. Bicyclic heteroaryl ring systems include fused ring systems, where two rings share two atoms, and spiro ring systems, where two rings share one atom. Heteroaryl groups within the scope of this definition include but are not limited to: azaindolyl, benzoimidazolyl, benzisoxazolyl, benzofuranyl, benzofurazanyl, benzopyrazolyl, benzotriazolyl, benzothiophenyl, benzothiazolyl, benzo[d]isothiazolyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazolinyl, isoxazolinyl, pyranyl, pyrazinyl, pyrazolyl, pyrrolyl, pyrazolopyrimidinyl, pyridazinyl, pyridyl, pyrimidyl, pyrimidinyl, pyrrolyl, quinazolinyl, quinolyl, quinoxalinyl, tetrazolyl, tetrazolopyridyl, thiadiazolyl, 5H-pyrrolo[3,4-b]pyridine, thiazolyl, thienyl, triazolyl, triazinyl, benzothiazolyl, benzothienyl, quinolinyl, quinazolinyl, and isoquinolinyl, and oxazolyl. If the heteroaryl contains nitrogen atoms, it is understood that the corresponding N-oxides thereof are also encompassed by this definition.

The term “heterocycloalkyl” as used herein refers to a stable and non-aromatic (including not fully aromatic, e.g., one double bond) 3- to 12-membered ring radical that comprises two to twelve ring carbon atoms and from one to six ring heteroatoms selected from nitrogen, oxygen, and sulfur. Whenever it appears herein, a numerical range such as “3 to 12” or “3-12” refers to each integer in the given range. For example, “3 to 12 ring atoms” means that the heterocycloalkyl group may consist of 3 ring atoms, 4 ring atoms, 5 ring atoms, etc., up to and including 12 ring atoms. In some embodiments, it is a 5 to 10 ring heterocycloalkyl. In some embodiments, it is a 4 to 10 ring heterocycloalkyl. In some embodiments, it is a 3 to 10 ring heterocycloalkyl. Unless stated otherwise specifically in the specification, the heterocycloalkyl radical may be a monocyclic, bicyclic, tricyclic, or tetracyclic ring system, which may include fused or bridged ring systems. The prefix aza, oxa or thia before the heterocyclyl root name means that at least a nitrogen, oxygen, or sulfur atom, respectively, is present as a ring atom. In some embodiments, the nitrogen or sulfur atom of the heterocycloalkyl can be optionally oxidized to the corresponding N-oxide, S-oxide (S═O) or S-dioxide (SO₂). One or more nitrogen atoms, if present, are optionally quaternized. The heterocycloalkyl radical is partially or fully saturated. The heterocycloalkyl may be attached to the rest of a molecule through any atom of the ring(s).

In one embodiment, a heterocycloalkyl group is monocyclic and has from about 3 to about 7 ring atoms. In another embodiment, a heterocycloalkyl group is monocyclic has from about 5 to about 8 ring atoms. In another embodiment, a heterocycloalkyl group is bicyclic and has from about 8 to about 11 ring atoms. In still another embodiment, a heterocycloalkyl group is monocyclic and has 5 or 6 ring atoms. In one embodiment, a heterocycloalkyl group is monocyclic. In another embodiment, a heterocycloalkyl group is bicyclic. In another embodiment, a heterocycloalkyl group is tricyclic. There are no adjacent oxygen and/or sulfur atoms present in the ring system.

Non-limiting examples of heterocycloalkyl rings include decahydroisoquinoline, dioxaspiro[4.5]decane, 2,5-diazabicyclo[2.2.1]heptyl, quinuclidinyl, oxetanyl, piperidyl, pyrrolidinyl, piperazinyl, morpholinyl, thiomorpholinyl, thiazolidinyl, 1,4-dioxanyl, tetrahydrofuranyl, tetrahydrothiophenyl, beta-lactam, gamma-lactam, delta-lactam, beta-lactone, gamma-lactone, delta-lactone, piperidinyl, 3-azabixyclo[3.1.0]hexyl, 2-azabicyclo[2.1.1]hexyl, 6-azaspiro[2.5]octanyl, azetidinyl, 2,3-dihydro-1H-indenyl, dihydro-1H-indenyl, 3H-spiro[benzofuran-2′,4′-piperidinyl, 2,3-dihydro-1H-pyrrolo[3,2,1-ij][1,6]naphthyridinyl, 3,4,6,7-tetrahydro-5H-imidazo[4,5-c]pyridyl, 3a,5,6,6a-tetrahydro-4H-pyrrolo[3,4-d]isoxazole, diazabicyclo[3.3.2]decanyl, 2,3,4,5,6,7-hexahydroisothiazolo[5,4-c]pyridyl, hexahydro-2H-pyrrolo[3,4-d]isothiazolyl, 3,9-diazabicyclo[3.3.2]decanyl, bicyclo[2,2,1]heptenyl, 2′,3′-dihydro-1′H-spiro[piperidine-4,4′-quinazolin], octahydropyrrolo[3,4-b][1,4]oxazinyl, (diazabicyclo[2.2.1]heptanyl), 2,5-diazabicyclo[2.2.1]heptanyl, tetrahydrobenzo[d]thiazolyl, 4,5,6,7-tetrahydrobenzo[d]thiazolyl, 2,3-dihydrobenzofuranyl, oxabicyclo[2.1.1]hexyl, dihydro-5H-pyrrolo[3,4-d]thiazolyl, 4,6-dihydro-5H-pyrrolo[3,4-d]thiazolyl, dihydro-5H-pyrrolo[3,4-d]oxazolyl, 4,6-dihydro-5H-pyrrolo[3,4-d]oxazolyl, dihydrothiazolo[5,4-c]pyridin-5(4H)-yl, 6,7-dihydrothiazolo[5,4-c]pyridin-5(4H)-yl, benzo[d]imidazolyl, 1H-enzo[d]imidazolyl, diazaspiro[4.4]nonanyl, and 2,7-diazaspiro[4.4]nonanyl, and pyrrolidinone, and oxides thereof and all isomers thereof. In one embodiment of the invention, heterocycloalkyl rings include: piperidyl, pyrrolidinyl, piperazinyl, morpholinyl, thiomorpholinyl, azeridinyl, azetidinyl.

“Nitro” means a —NO₂ group.

“Oxo” means an oxygen atom connected to another atom by a double bond and is represented by “═O” herein.

The term “oxy” means an oxygen (O) atom.

“Quaternary amine” or “quaternary ammonium” means positively charged radical having four functional groups attached to a nitrogen atom, such as N,N,N-trimethyl ammonium

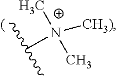

and N,N,N-trimethylpropan-1-aminium

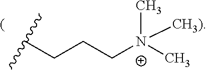

“Sulfinyl” means a bivalent functional group —S(═O)—.

“Sulfonyl” means a bivalent functional group —SO₂—.

“Tertiary amine” means an amine in which three carbon atoms are attached to the amino nitrogen.

The term “thio” means a sulfur (S) atom.

“Trialkylammonium” means an amino containing three alkyl groups attached to the amino nitrogen or (C₁₋₆ alkyl)₃N⁺(C₀₋₆ alkyl)-, such as N,N,N-trimethyl ammonium

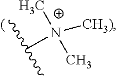

and N,N,N-trimethylpropan-1-aminium

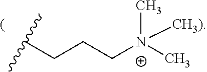

“Urea” means —NR—C(═O)—NR₂ where R may independently be hydrogen, alkyl, aryl, for example, —NHCONH₂.

By “pharmaceutically acceptable” is meant that the ingredients of the pharmaceutical composition must be compatible with each other and not deleterious to the recipient thereof.

Where any amine is present in the compound, the nitrogen (N) atom may be optionally in the form of a quaternary amine having one or more appropriate additional substitutions, as further described herein.

When any variable (e.g., n, R^{a}, R^{b}, etc.) occurs more than one time in any constituent or in Formula I, its definition on each occurrence is independent of its definition at every other occurrence. Also, combinations of substituents and/or variables are permissible only if such combinations result in stable compounds.

When any ring atom is specified as being optionally substituted with, or in a specified form, for example, sulfur (S) substituted with oxo groups, or nitrogen (N) in the form of a N-oxide, this does not preclude the substitution of any ring atom with the other listed optional substituents when not substituted with oxo groups or in the form of a N-oxide.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds.

By “stable compound” or “stable structure” is meant a compound that is sufficiently robust to survive isolation to a useful degree of purity from a reaction mixture, and formulation into an efficacious therapeutic agent. The compounds of the present invention are limited to stable compounds embraced by Formula I.

The term “compound” refers to the compound and, in certain embodiments, to the extent they are stable, any hydrate or solvate thereof. A hydrate is the compound complexed with water, and a solvate is the compound complexed with an organic solvent.

The term “in substantially purified form”, as used herein, refers to the physical state of a compound after the compound is isolated from a synthetic process (e.g., from a reaction mixture), a natural source, or a combination thereof. The term “in substantially purified form” also refers to the physical state of a compound after the compound is obtained from a purification process or processes described herein or well-known to the skilled artisan (e.g., chromatography, reversed-phase preparative HPLC, recrystallization, and the like), in sufficient purity to be characterizable by standard analytical techniques described herein or well-known to the skilled artisan.

It should also be noted that any carbon as well as heteroatom with unsatisfied valences in the text, schemes, examples, and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al., Protective Groups in Organic Synthesis (1991), Wiley, New York.

Under standard nomenclature used throughout this disclosure, the terminal portion of the designated side chain is preceded by the adjacent functionality toward the point of attachment. For example, a “(C₁₋₅ alkyl)carbonylamino(C₁₋₆ alkyl)” substituent is equivalent to

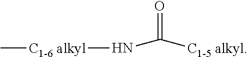

Structural representations of compounds having substituents terminating with a methyl group may display the terminal methyl group either using the characters “Me”, “-Me”, “CH₃”, “—CH₃” or using a straight line representing the presence of the methyl group, e.g., [symbol] i.e.,

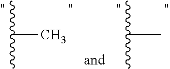

have equivalent meanings.

For variable definitions containing terms having repeated terms, e.g., (CRᵢRⱼ)ᵣ, where r is the integer 2, Rᵢ is a defined variable, and Rⱼ is a defined variable, the value of R¹ may differ in each instance in which it occurs, and the value of Rⱼ may differ in each instance in which it occurs. For example, if Rᵢ and Rⱼ are independently selected from the group consisting of methyl, ethyl, propyl, and butyl, then (CRᵢRⱼ)₂ can be

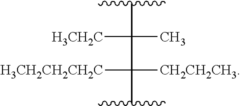

Unless expressly stated to the contrary, all ranges cited herein are inclusive. For example, a heteroaromatic ring described as containing from “1 to 4 heteroatoms” means the ring can contain, 1, 2, 3 or 4 heteroatoms. It is also to be understood that any range cited herein includes within its scope all of the sub-ranges within that range. Thus, for example, a heterocyclic ring described as containing from “1 to 4 heteroatoms” is intended to include as aspects thereof, heterocyclic rings containing 2 to 4 heteroatoms, 3 or 4 heteroatoms, 1 to 3 heteroatoms, 2 or 3 heteroatoms, 1 or 2 heteroatoms, 1 heteroatom, 2 heteroatoms, 3 heteroatoms, and 4 heteroatoms. Similarly, C₁₋₆ or C₁₋₆ or C₁-C₆ when used with a chain, for example an alkyl chains means that the chain can contain 1, 2, 3, 4, 5, or 6 carbon atoms. It also includes all ranges contained therein including C₁-C₅, C₁-C₄, C₁-C₃, C₁-C₂, C₂-C₆, C₃-C₆, C₄-C₆, C₅-C₆, and all other possible combinations.

In choosing compounds of the present invention, one of ordinary skill in the art will recognize that the various substituents, i.e., R¹, R^{A}, R^{2b}, etc., are to be chosen in conformity with well-known principles of chemical structure connectivity and stability.

As used herein, the term “composition” is intended to encompass a product comprising the specified ingredients in the specified amounts, as well as any product which results from combination of the specified ingredients in the specified amounts.

When any variable (e.g., R^{2a}) occurs more than one time in any constituent or in Formula (I) or other generic formulas herein, its definition on each occurrence is independent of its definition at every other occurrence. Combinations of substituents and/or variables are permissible only if such combinations result in stable compounds. In choosing compounds of the present disclosure, one of ordinary skill in the art will recognize that the various substituents, e.g., R^{2a}, are to be chosen in conformity with well-known principles of chemical structure connectivity and stability. Unless expressly stated to the contrary, substitution by a named substituent is permitted on any atom in a ring (e.g., aryl, heteroaryl ring, or saturated heteroaryl ring) provided such ring substitution is chemically allowed and results in a stable compound. A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

The term “substituted” shall be deemed to include multiple degrees of substitution by a named substituent. Where multiple substituent moieties are disclosed or claimed, the substituted compound can be independently substituted by one or more of the disclosed or claimed substituent moieties, singly or pluraly. By independently substituted, it is meant that the (two or more) substituents can be the same or different.

The wavy line [symbol] , as used herein, indicates a point of attachment to the rest of the compound.

Some of the compounds described herein may exist as tautomers which have different points of attachment of hydrogen accompanied by one or more double bond shifts. For example, a ketone and its enol form are keto-enol tautomers. The individual tautomers as well as mixtures thereof are encompassed with compounds of the present disclosure.

In the compounds of the disclosure, the atoms may exhibit their natural isotopic abundances, or one or more of the atoms may be artificially enriched in a particular isotope having the same atomic number, but an atomic mass or mass number different from the atomic mass or mass number predominantly found in nature. The present disclosure as described and claimed herein is meant to include all suitable isotopic variations of the compounds of the disclosure and embodiments thereof. For example, different isotopic forms of hydrogen (H) include protium (¹H) and deuterium (²H, also denoted herein as D). Protium is the predominant hydrogen isotope found in nature. Enriching for deuterium may afford certain therapeutic advantages, such as increasing in vivo half-life or reducing dosage requirements or may provide a compound useful as a standard for characterization of biological samples. Isotopically-enriched compounds of the disclosure can be prepared without undue experimentation by conventional techniques well-known to those skilled in the art or by processes analogous to those described in the Schemes and Examples herein using appropriate isotopically-enriched reagents and/or intermediates.

The term “pharmaceutically acceptable salts” refers to salts prepared from pharmaceutically acceptable non-toxic bases or acids. When the compound of the present disclosure is acidic (or has a functional group which may be anionic), its corresponding salt can be conveniently prepared from pharmaceutically acceptable non-toxic bases, including inorganic bases and organic bases. Examples of suitable inorganic cations include, but are not limited to, alkali metal ions such as Li⁺, Na⁺, and K⁺, alkaline earth metal cations such as Ca²⁺, and Mg²⁺, and other cations such as Al³⁺ and Zn²⁺. Examples of suitable organic cations include, but are not limited to, ammonium ion (i.e., NH₄+) and substituted ammonium ions. Examples of suitable substituted ammonium ions are those derived from methylamine, ethylamine, diethylamine, triethylamine and ethylenediamine. When a compound of the present disclosure is basic, its corresponding salt can be conveniently prepared from pharmaceutically acceptable non-toxic acids, including inorganic acids and organic acids. Example of such acid addition salts include salts formed from hydrohalic acids (e.g., hydrochloric, hydrobromic, hydroiodic), formic acid, acetic acid, capric acid, and citric acid. Salts containing acetate, formate, caprate, chloride, or sodium salts are typical for use with the compounds of the present disclosure. In some embodiments, salts of compounds of the present disclosure can be formed by exchange well-known to those of ordinary skill in the art, such as by anion exchange, e.g., replacement of trifluoroacetate ions with chloride ions.

Furthermore, compounds of the present disclosure may exist in amorphous form and/or one or more crystalline forms, and as such all amorphous and crystalline forms and mixtures thereof of the compounds of Formula (I), including the Examples, are intended to be included within the scope of the present disclosure. In addition, some of the compounds of the instant disclosure may form solvates with water (i.e., a hydrate) or common organic solvents such as, but not limited to, acetic acid or acetonitrile. Such solvates and hydrates, particularly the pharmaceutically acceptable solvates and hydrates, of the instant compounds are likewise encompassed within the scope of this disclosure, along with un-solvated and anhydrous forms.

Any pharmaceutically acceptable pro-drug modification of a compound of this disclosure which results in conversion in vivo to a compound within the scope of this disclosure is also within the scope of this disclosure.

The present disclosure also relates to processes for the preparation of the compounds of Formula (I) which are described in the following Examples and by which the compounds of the disclosure are obtainable.

“Treatment” and “treating” refer to all processes in which there may be a slowing, interrupting, arresting, controlling, or stopping of the progression of a disease or disorder described herein. The terms do not necessarily indicate a total elimination of all disease or disorder symptoms.

“Preventing” or “prophylaxis” as used herein, refers to reducing the likelihood of contracting disease or disorder described herein, or reducing the severity of a disease or disorder described herein.

The terms “therapeutically effective (or efficacious) amount” and similar descriptions such as “an amount efficacious for treatment” or “an effective dose” are intended to mean that amount of a compound of the disclosure that will elicit the biological or medical response of a tissue, system, animal, or human that is being sought by a researcher, veterinarian, medical doctor or other clinician. In a preferred embodiment, the term “therapeutically effective amount” means an amount of a compound of the disclosure that alleviates at least one clinical symptom in a human patient. The terms “prophylactically effective (or efficacious) amount” and similar descriptions such as “an amount efficacious for prevention” are intended to mean that amount of a compound of the disclosure that will prevent or reduce the risk of occurrence of the biological or medical event that is sought to be prevented in a tissue, a system, animal or human by a researcher, veterinarian, medical doctor, or other clinician.

### Dosages of the Compounds of the Present Disclosure

The dosage regimen utilizing a compound of the present disclosure is selected in accordance with a variety of factors including type, species, age, weight, sex, and medical condition of the patient; the severity of the condition to be treated; the potency of the compound chosen to be administered; the route of administration; and the renal and hepatic function of the patient. A consideration of these factors is well within the purview of the ordinarily skilled clinician for the purpose of determining the therapeutically effective or prophylactically effective dosage amount needed to prevent, counter, or arrest the progress of the condition. It is understood that a specific daily dosage amount can simultaneously be both a therapeutically effective amount, e.g., for treatment of an immunological condition, and a prophylactically effective amount, e.g., for prevention of an immunological condition.

While individual needs vary, determination of optimal ranges of effective amounts of the compound of the present disclosure is within the skill of the art. For administration to a human in the curative or prophylactic treatment of the conditions and disorders identified herein, for example, typical dosages of the compounds of the present disclosure can be about 0.05 mg/kg/day to about 1000 mg/kg/day. In some embodiments, a patient is administered from about 5 mg/day to about 1000 mg/day, such as from 10 mg/day, 20 mg/day, 30 mg/day, 40 mg/day, 50 mg/day, 60 mg/day, 70 mg/day, 80 mg/day, 90 mg/day, 100 mg/day, 200 mg/day, 300 mg/day, 400 mg/day, 500 mg/day, 600 mg/day, 700 mg/day, 800 mg/day, 900 mg/day, or 1000 mg/day of a compound of the present disclosure. In certain embodiments, a patient is administered from about 0.2 mg/kg to about 5 mg/kg, such as from 0.5 mg/kg, 0.75 mg/kg, 1.0 mg/kg, 1.25 mg/kg, 1.5 mg/kg, 2.5 mg/kg, 3.0 mg/kg, 3.5 mg/kg, 4.0 mg/kg, 4.5 mg/kg, 5 mg/kg 5.5 mg/kg, 6.0 mg/kg, 7.0 mg/kg, 7.5 mg/kg, 8 mg/kg, 8.5 mg/kg, 9.0 mg/kg, 9.5 mg/kg or 10 mg/kg of a compound of the present disclosure. Such doses may be administered in a single dose or may be divided into multiple doses.

### Pharmaceutical Compositions

The compounds of the disclosure and their pharmaceutically acceptable salts can be administered to animals, preferably to mammals, and particularly to humans, as pharmaceuticals by themselves, in mixtures with one another or in the form of pharmaceutical compositions. The term “subject” or “patient” includes animals, preferably mammals and especially humans, who use the instant active agents for the prevention or treatment of a medical condition.

Administering of the drug to the subject includes both self-administration and administration to the patient by another person. The subject may be in need of or desire treatment for an existing disease or medical condition or may be in need of or desire prophylactic treatment to prevent or reduce the risk of occurrence of the disease or medical condition. As used herein, a subject “in need” of treatment of an existing condition or of prophylactic treatment encompasses both a determination of need by a medical professional as well as the desire of a patient for such treatment.

The present disclosure therefore also provides the compounds of the disclosure and their pharmaceutically acceptable salts for use as pharmaceuticals, their use for selectively modulating the activity of TNFR1, and in particular, their use in the therapy and prophylaxis of the below-mentioned diseases or disorders as well as their use for preparing medicaments for these purposes. In certain embodiments, the compounds of the disclosure and their pharmaceutically acceptable salts block TNFα signaling through TNFR1.

Furthermore, the present disclosure provides pharmaceutical compositions which comprise as active component an effective dose of at least one compound of the disclosure and/or a pharmaceutically acceptable salt thereof and a customary pharmaceutically acceptable carrier, i.e., one or more pharmaceutically acceptable carrier substances and/or additives.

Thus, the present disclosure provides, for example, said compound and its pharmaceutically acceptable salts for use as pharmaceutical compositions which comprise as active component an effective dose of the compound of the disclosure and/or a pharmaceutically acceptable salt thereof and a customary pharmaceutically acceptable carrier, and the uses of said compound and/or a pharmaceutically acceptable salt thereof in the therapy or prophylaxis of the below-mentioned diseases or disorders, e.g., inflammatory bowel diseases (IBD), rheumatoid arthritis, juvenile rheumatoid arthritis, psoriaris, psoriatic arthritis, ankylosing spondylitis, non-radiographic axial spondyloarthritis and hidradenitis suppurativa as well as their use for preparing medicaments for these purposes.

The pharmaceutical compositions according to the disclosure can be administered orally, for example, in the form of pills, tablets, lacquered tablets, sugar-coated tablets, granules, hard and soft gelatin capsules, aqueous, alcoholic, or oily solutions, syrups, emulsions or suspensions, or rectally, for example, in the form of suppositories. Administration can also be carried out parenterally, for example, subcutaneously, intramuscularly, or intravenously in the form of solutions or suspension for injection or infusion.

Other suitable administration forms are, for example, percutaneous or topical administration, for example, in the form of ointments, tinctures, sprays or transdermal therapeutic systems, or, for example, microcapsules, implants or rods. The preferred administration form depends, for example, on the disease to be treated and on its severity.

The present disclosure also provides pharmaceutical compositions comprising a compound of Formula (I). The compound of Formula (I) can be used in combination with any suitable pharmaceutical carrier or excipient. Such pharmaceutical compositions comprise a therapeutically effective amount of one or more compounds of Formula (I), and pharmaceutically acceptable excipient(s) and/or carrier(s). The specific pharmaceutic composition will suit the mode of administration. In particular aspects, the pharmaceutical acceptable carrier may be water or a buffered solution.

Excipients included in the pharmaceutical compositions have different purposes depending, for example on the nature of the drug, and the mode of administration. Examples of generally used excipients include, without limitation: saline, buffered saline, dextrose, water-for-infection, glycerol, ethanol, and combinations thereof, stabilizing agents, solubilizing agents and surfactants, buffers and preservatives, tonicity agents, bulking agents, lubricating agents (such as talc or silica, and fats, such as vegetable stearin, magnesium stearate or stearic acid), emulsifiers, suspending or viscosity agents, inert diluents, fillers (such as cellulose, dibasic calcium phosphate, vegetable fats and oils, lactose, sucrose, glucose, mannitol, sorbitol, calcium carbonate, and magnesium stearate), disintegrating agents (such as crosslinked polyvinyl pyrrolidone, sodium starch glycolate, cross-linked sodium carboxymethyl cellulose), binding agents (such as starches, gelatin, cellulose, methyl cellulose or modified cellulose such as microcrystalline cellulose, hydroxypropyl cellulose, sugars such as sucrose and lactose, or sugar alcohols such as xylitol, sorbitol or maltitol, polyvinylpyrrolidone and polyethylene glycol), wetting agents, antibacterials, chelating agents, coatings (such as a cellulose film coating, synthetic polymers, shellac, corn protein zein or other polysaccharides, and gelatin), preservatives (including vitamin A, vitamin E, vitamin C, retinyl palmitate, and selenium, cysteine, methionine, citric acid and sodium citrate, and synthetic preservatives, including methyl paraben and propyl paraben), sweeteners, perfuming agents, flavoring agents, coloring agents, absorption enhancers, administration aids, and combinations thereof.

Carriers are compounds and substances that improve and/or prolong the delivery of an active ingredient to a subject in the context of a pharmaceutical composition. Carriers may serve to prolong the in vivo activity of a drug or slow the release of the drug in a subject, using controlled-release technologies. Carriers may also decrease drug metabolism in a subject and/or reduce the toxicity of the drug. Carriers can also be used to target the delivery of the drug to particular cells or tissues in a subject. Common carriers (both hydrophilic and hydrophobic carriers) include fat emulsions, lipids, PEGylated phospholipids, PEGylated liposomes, PEGylated liposomes coated via a PEG spacer with a cyclic RGD peptide, liposomes and lipospheres, microspheres (including those made of biodegradable polymers or albumin), polymer matrices, biocompatible polymers, protein-DNA complexes, protein conjugates, erythrocytes, vesicles, nanoparticles, and side chains for hydrocarbon stapling. The aforementioned carriers can also be used to increase cell membrane permeability of the compounds of Formula (I). In addition to their use in the pharmaceutical compositions of the present disclosure, carriers may also be used in compositions for other uses, such as research uses in vitro (e.g., for delivery to cultured cells) and/or in vivo.

Pharmaceutical compositions adapted for oral administration may be presented as discrete units such as capsules or tablets; as powders or granules; as solutions, syrups, or suspensions (in aqueous or non-aqueous liquids; or as edible foams or whips; or as emulsions). Suitable excipients for tablets or hard gelatin capsules include lactose, maize starch, or derivatives thereof, stearic acid or salts thereof. Suitable excipients for use with soft gelatin capsules include for example vegetable oils, waxes, fats, semi-solid, or liquid polyols etc. For the preparation of solutions and syrups, excipients which may be used include for example water, polyols, and sugars. For the preparation of suspensions oils, e.g., vegetable oils, may be used to provide oil-in-water or water-in-oil suspensions. Excipients which promote absorption from the gastrointestinal tract, e.g., permeation enhancers, such as sodium caprate can be included. In certain situations, delayed release preparations may be advantageous and compositions which can deliver the compounds of the present disclosure in a delayed or controlled release manner may also be prepared. Prolonged gastric residence brings with it the problem of degradation by the enzymes present in the stomach and so enteric-coated capsules may also be prepared by standard techniques in the art where the active substance for release lower down in the gastro-intestinal tract.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6):318 (1986).

Pharmaceutical compositions adapted for topical administration may be formulated as ointments, creams, suspensions, lotions, powders, solutions, pastes, gels, sprays, aerosols, or oils. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles, and mouth washes.

Pharmaceutical compositions adapted for rectal administration may be presented as suppositories or enemas.

Pharmaceutical compositions adapted for nasal administration wherein the carrier is a solid include a coarse powder having a particle size for example in the range 20 to 500 microns which is administered in the manner in which snuff is taken, i.e., by rapid inhalation through the nasal passage from a container of the powder held close up to the nose. Suitable compositions wherein the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers, or insufflators.

Pharmaceutical compositions adapted for vaginal administration may be presented as pessaries, tampons, creams, gels, pastes, foams, or spray formulations.

Pharmaceutical compositions adapted for parenteral administration include aqueous and non-aqueous sterile injection solution which may contain antioxidants, buffers, bacteriostats and solutes which render the formulation substantially isotonic with the blood of the intended recipient; and aqueous and non-aqueous sterile suspensions which may include suspending agents and thickening agents. Excipients which may be used for injectable solutions include water-for-injection, alcohols, polyols, glycerin, and vegetable oils, for example. The compositions may be presented in unit-dose or multi-dose containers, for example sealed ampoules and vials, and may be stored in a freeze-dried (lyophilized) condition requiring only the addition of the sterile liquid carrier, for example water or saline for injections, immediately prior to use. Extemporaneous injection solutions and suspensions may be prepared from sterile powders, granules, and tablets. The pharmaceutical compositions may contain preserving agents, solubilizing agents, stabilizing agents, wetting agents, emulsifiers, sweeteners, colorants, odorants, salts (substances of the present disclosure may themselves be provided in the form of a pharmaceutically acceptable salt), buffers, coating agents or antioxidants. They may also contain therapeutically active agents in addition to the compounds of the present disclosure.

### Methods of Using the Compounds of the Disclosure

The present application provides a method of TNFR1-mediated cell signaling comprising contacting a cell with a compound of the disclosure or a pharmaceutically acceptable salt thereof. Binding to human TNFR1 and TNFR2 and selectivity can be assessed by SPR. Affinity to human TNFR1 can be assessed using a time-resolved fluorescence resonance energy transfer (TR-FRET) binding assay. Inhibition of TNFR1-mediated cell signaling can be assessed by detecting decreases in the levels of downstream NF-κB signal transduction pathway.

The present application also provides methods of using the compounds of the disclosure (or their pharmaceutically acceptable salts) or pharmaceutical compositions containing such compounds to treat disease conditions, including but not limited to, conditions implicated by TNFR1.

In some embodiments, the present disclosure provides a method of treating IBD and other TNFα-driven inflammatory diseases, the method comprising administering a therapeutically effective amount a compound of the disclosure (or a pharmaceutically acceptable salt thereof) or any of the foregoing pharmaceutical compositions comprising such a compound to a subject in need of such treatment. In some embodiments, the TNFα-driven inflammatory disease is IBD. In some embodiments, the TNFα-driven inflammatory disease is ulcerative colitis. In some embodiments the TNFα-driven inflammatory disease is Crohn's disease. In some embodiments, the TNFα-driven inflammatory disease is rheumatoid arthritis. In some embodiments, the TNFα-driven inflammatory disease is juvenile rheumatoid arthritis. In some embodiments, the TNFα-driven inflammatory disease is psoriasis. In some embodiments, the TNFα-driven inflammatory disease is psoriatic arthritis. In some embodiments, the TNFα-driven inflammatory disease is ankylosing spondylitis. In some embodiments, the TNFα-driven inflammatory disease is non-radiographic axial spondyloarthritis. In some embodiments, the TNFα-driven inflammatory disease is hidradenitis suppurativa.

In some embodiments, the present disclosure provides a method of treating an inflammatory bowel disease, the method comprising administering a therapeutically effective amount a compound of the disclosure (or a pharmaceutically acceptable salt thereof) or any of the foregoing pharmaceutical compositions comprising such a compound to a subject in need of such treatment.

In some embodiments, the present disclosure provides a method of treating ulcerative colitis, the method comprising administering a therapeutically effective amount a compound of the disclosure (or a pharmaceutically acceptable salt thereof) or any of the foregoing pharmaceutical compositions comprising such a compound to a subject in need of such treatment.

In some embodiments, the present disclosure provides a method of treating Crohn's disease, the method comprising administering a therapeutically effective amount a compound of the disclosure (or a pharmaceutically acceptable salt thereof) or any of the foregoing pharmaceutical compositions comprising such a compound to a subject in need of such treatment.

In some embodiments, the present disclosure provides a method of treating rheumatoid arthritis, the method comprising administering a therapeutically effective amount a compound of the disclosure (or a pharmaceutically acceptable salt thereof) or any of the foregoing pharmaceutical compositions comprising such a compound to a subject in need of such treatment.

In some embodiments, the present disclosure provides a method of treating juvenile rheumatoid arthritis, the method comprising administering a therapeutically effective amount a compound of the disclosure (or a pharmaceutically acceptable salt thereof) or any of the foregoing pharmaceutical compositions comprising such a compound to a subject in need of such treatment.

In some embodiments, the present disclosure provides a method of treating psoriasis, the method comprising administering a therapeutically effective amount a compound of the disclosure (or a pharmaceutically acceptable salt thereof) or any of the foregoing pharmaceutical compositions comprising such a compound to a subject in need of such treatment.

In some embodiments, the present disclosure provides a method of treating psoriatic arthritis, the method comprising administering a therapeutically effective amount a compound of the disclosure (or a pharmaceutically acceptable salt thereof) or any of the foregoing pharmaceutical compositions comprising such a compound to a subject in need of such treatment.

In some embodiments, the present disclosure provides a method of treating ankylosing spondylitis, the method comprising administering a therapeutically effective amount a compound of the disclosure (or a pharmaceutically acceptable salt thereof) or any of the foregoing pharmaceutical compositions comprising such a compound to a subject in need of such treatment.

In some embodiments, the present disclosure provides a method of treating non-radiographic axial spondyloarthritis, the method comprising administering a therapeutically effective amount a compound of the disclosure (or a pharmaceutically acceptable salt thereof) or any of the foregoing pharmaceutical compositions comprising such a compound to a subject in need of such treatment.

In some embodiments, the present disclosure provides a method of treating hidradenitis suppurativa, the method comprising administering a therapeutically effective amount a compound of the disclosure (or a pharmaceutically acceptable salt thereof) or any of the foregoing pharmaceutical compositions comprising such a compound to a subject in need of such treatment.

In some embodiments, the present disclosure provides a method of treating IBD and other TNFα-driven inflammatory diseases comprising administering a therapeutically effective amount a compound of the disclosure (or a pharmaceutically acceptable salt thereof) to a subject in need of such treatment, wherein said IBD and other TNFα-driven inflammatory diseases are selected from ulcerative colitis, Crohn's disease, rheumatoid arthritis, juvenile rheumatoid arthritis, psoriasis, psoriatic arthritis, ankylosing spondylitis, non-radiographic axial spondyloarthritis, and hidradenitis suppurativa.

One embodiment of the invention relates to the use of a compound of formula (I) or a pharmaceutically acceptable salt thereof in therapy.

One embodiment of the invention relates to the use of a compound of formula (I) or a pharmaceutically acceptable salt thereof for treating IBD and other TNFα-driven inflammatory diseases selected from ulcerative colitis, Crohn's disease, rheumatoid arthritis, juvenile rheumatoid arthritis, psoriasis, psoriatic arthritis, ankylosing spondylitis, non-radiographic axial spondyloarthritis, and hidradenitis suppurativa.

One embodiment relates to the use of a compound of formula (I) or a pharmaceutically acceptable salt thereof for treating IBD and other TNFα-driven inflammatory diseases selected from ulcerative colitis and Crohn's disease.

One embodiment relates to the use of a compound of formula (I) or a pharmaceutically acceptable salt thereof for treating IBD and other TNFα-driven inflammatory diseases selected from rheumatoid arthritis, juvenile rheumatoid arthritis, psoriasis, psoriatic arthritis, ankylosing spondylitis, non-radiographic axial spondyloarthritis, and hidradenitis suppurativa. COMBINATION THERAPIES

One or more additional pharmacologically active agents may be administered in combination with a compound of the disclosure. An additional active agent (or agents) is intended to mean a pharmaceutically active agent (or agents) that is active in the body, including prodrugs that convert to pharmaceutically active form after administration, which are different from the compound of Formula I, and also includes free-acid, free-base, and pharmaceutically acceptable salts of said additional active agents.

Examples of additional active agents which may be employed include but are not limited to, anti-TNFα biologics, such as, for example, methotrexate, azathioprine, 6-mercapto purine, anti-IL-23 agents, anti-α4ρ7 agents like vedolizumab, biased IL-2Rα agonists in clinical development.

### METHODS OF SYNTHESIS

### General Procedures to Access Building Blocks and Monomers

The compounds of the present invention can be prepared according to the procedures of the following schemes and specific examples, or modifications thereof, using readily available starting materials, appropriate materials and reagents and conventional synthetic procedures and are further exemplified by the following specific examples. In these reactions, it is also possible to make use of variants which are themselves known to those of ordinary skill in this art but are not mentioned in greater detail. The general procedures for making the compounds claimed in this invention can be readily understood and appreciated by one skilled in the art from viewing the following schemes. The examples also include methods for testing such compounds in biophysical, biochemical, and cellular assays. The compounds illustrated in the examples are not, however, to be construed as forming the only genus that is considered as the disclosure.

Unless otherwise specifically indicated, all reagents are commercially available, known in the literature, or readily synthesized by one skilled in the art. The general route applied to the synthesis of compounds of Formula I is described in the Schemes that follow. In some instances, the order of carrying out the reaction steps in the schemes may be varied to facilitate the reaction or to avoid unwanted reaction products. Additionally, various protecting group strategies familiar to one skilled in the art of organic synthesis and solid phase peptide synthesis may be employed to facilitate the reaction, to improve yield and purity, or to avoid unwanted reaction products.

All reagents and solvents were purchased from commercial sources and used without further purification unless otherwise noted. All temperatures are in degrees Celsius (° C.), and ambient temperature or room temperature (RT) is 20° C. Most compounds were purified by reversed-phase preparative high-performance liquid chromatography (HPLC) or medium-pressure liquid chromatography (MPLC) on silica gel. The course of the reactions was followed by liquid chromatography/mass spectrometry (LC-MS) or Ultra performance liquid chromatography/mass spectrometry (UPLC-MS); electrospray ionization (ESI); UV detection at 254 nm). Proton, fluorine, and carbon magnetic resonance (¹H, ¹⁹F and ¹³C NMR) spectra were recorded on a 300, 400, 500, or 600 MHz Varian or Bruker spectrometer, and chemical shifts are reported in parts per million (ppm) relative to tetramethylsilane and referenced to residual solvent. Coupling constants are reported in hertz. ¹H NMR data are reported as given here: chemical shift (multiplicity [singlet (s), doublet (d), triplet (t), quartet (q), doublet of doublets (dd), doublet of triplets (dt), triplet of doublets (td), triplet of triplets (tt), doublet of doublet of doublets (ddd), multiplet (m), and broad singlet (br. s)], coupling constant [Hz] and integration). Reactions sensitive to moisture or air were performed under nitrogen or argon using anhydrous solvents and reagents. The progress of reactions was determined by either analytical thin layer chromatography (TLC) usually performed with pre-coated TLC plates (E. Merck, Darmstadt, Germany), silica gel 60F-254, layer thickness 0.25 mm or liquid chromatography-mass spectrometry (LC-MS).

Unless otherwise indicated, when ratios of compounds (such as for examples solvents) are given, the ratio is on a volume-to-volume basis. For example, solvent gradient ranging from 100% hexanes to 50% EtOAc/hexanes means a gradient starting from a mixture of 100 parts by volume of hexanes varying to mixture of 50 parts by volume ethyl acetate to 50 parts by volume of hexanes.

The term “w/w” means weight of compound to total weight. For example, NaH 60% w/w means 60 parts by weight NaH to 100 parts total weight.

The following examples are provided so that the invention might be more fully understood. These examples are illustrative only and should not be construed as limiting the invention in any way. Wherein a racemic mixture is produced, the enantiomers may be separated using SFC reversed or normal phase chiral resolution conditions either after isolation of the final product or at a suitable Intermediate, followed by processing of the single isomers individually. It is understood that alternative methodologies may also be employed in the synthesis of these key intermediates and examples. Asymmetric methodologies (e.g., chiral catalysis, auxiliaries, biocatalytic process) may be used where possible and appropriate. The exact choice of reagents, solvents, temperatures, and other reaction conditions depends upon the nature of the intended product.

### Abbreviations

The following abbreviations are used throughout the text:

| Abbreviation | Definition |
|---|---|
| 1Inda | (S)-2-amino-3-(1H-indazol-1-yl)propanoic acid |
| 1Inda7az | (S)-2-amino-3-(1H-pyrazolo[3,4-b]pyridin-1-yl)propanoic acid |
| 1Nal | 3-(1-Naphthyl)-L-alanine |
| 1R4SAcpc | (1R,4S)-4-aminocyclopent-2-ene-1-carboxylic acid |
| 1S4RAcpc | (1S,4R)-4-aminocyclopent-2-ene-1-carboxylic acid |
| 2BrPr | 2-Methylacetyl cross-linker |
| 2CTC | 2-Chlorotrityl chloride resin |
| 2Nal | 3-(2-Naphthyl)-L-alanine |
| 3Inda | (S)-2-Amino-3-(2H-indazol-3-yl)propanoic acid |
| 3Pal | 3-Pyridylalanine or 3-pyridyl-L-alanine |
| 3Pal4CO2H | (S)-5-(2-Amino-2-carboxyethyl)picolinic acid |
| 4CzIPN | 1,2,3,5-Tetrakis(carbazol-9-yl)-4,6-dicyanobenzene |
| 4OMe3Pal | (S)-2-Amino-3-(6-methoxypyridin-3-yl)propanoic acid |
| 4Pal | 4-Pyridylalanine or 4-pyridyl-L-alanine |
| 4Pal2F | (S)-2-Amino-3-(3-fluoropyridin-4-yl)propanoic acid |
| 4Pal3NH2 | (S)-2-Amino-3-(2-aminopyridin-4-yl)propanoic acid |
| 4PyridaAla | (S)-2-Amino-3-(pyridazin-4-yl)propanoic acid |
| 4PyrimAla | (S)-2-Amino-3-(pyrimidin-4-yl)propanoic acid |
| 4Quin | (S)-2-Amino-3-(quinolin-4-yl)propanoic acid |
| 2SBrBu | (S)-2-Ethylacetyl cross-linker |
| 2SBrPr | (S)-2-Methylacetyl cross-linker |
| A or Ala | L-Alanine |
| AA | Amino acid |
| AbuCN | (S)-2-amino-4-cyanobutanoic acid |
| Ac | Acetyl |
| Ac₂O | Acetic anhydride |
| Acetonitrile-d₃ | Trideuteroacetonitrile |
| ACN | Acetonitrile |
| AcOH | Acetic acid |
| ADA | Anti-drug antibodies |
| Aea | 2-(2-Aminoethoxy)acetic acid |
| AEF | (S)-2-Amino-3-[4-(2-aminoethoxy)phenyl]propanoic acid |
| Ahp | (S)-2-Aminoheptanoic acid |
| AhpF3 | (S)-2-Amino-7,7,7-trifluoroheptanoic acid |
| AIBN | 2,2′-Azobis(2-methylpropionitrile) |
| Ala4IsoindOne | (S)-2-Amino-3-(3-oxoisoindolin-5-yl)propanoic acid |
| AlaPent | (S)-2-Amino-3-(1-bicyclo[1.1.1]pentanyl)propanoic acid |
| AlapentOH | (S)-2-Amino-3-(3-hydroxy-1-bicyclo[1.1.1]pentanyl)propanoic acid |
| AlaPiperaz | (S)-2-Amino-3-(piperazin-1-yl)propanoic acid |
| AlaPyrim4COOH | (S)-5-(2-Amino-2-carboxyethyl)pyrimidine-2-carboxylic acid |
| AlaPyrim4NH2 | (S)-2-Amino-3-(2-aminopyrimidin-5-yl)propanoic acid |
| Alloc | Allyl carbamate |
| alT | L-allo-Threonine or (2S,3S)-2-amino-3-hydroxybutanoic acid |
| aMeS | α-Methyl-L-serine |
| aMeW | α-Methyl-L-tryptophan |
| anh. | Anhydrous |
| Anon | (S)-2-Aminononanoic acid |
| Apm | (S)-2-Aminoheptanedioic acid |
| aq. | Aqueous |
| Ar | Argon |
| B(C₆F₅)₃ | Tris(pentafluorophenyl)borane |
| BAIB | (Diacetoxyiodo)benzene |
| Bav | (S)-2,3-Diamino-3-methylbutanoic acid |
| BCP3CO2H | (S)-3-(2-Amino-2-carboxyethyl)bicyclo[1.1.1]pentane-1-carboxylic acid |
| BF₃•Et₂O | Boron trifluoride diethyl etherate |
| bhcLeu | (1R,3S)-3-Aminocyclopentane-1-carboxylic acid |
| bhcLeut45diOH | (1S,2R,3S,4R)-4-Amino-2,3-dihydroxycyclopentane-1-carboxylic acid |
| bhcLeut4OH | (1S,3R,4R)-3-Amino-4-hydroxycyclopentane-1-carboxylic acid |
| Bhv | (S)-2-Amino-3-hydroxy-3-methylbutanoic acid |
| Biotin | D-Biotin |
| bMe4Pal | (2S)-2-Amino-3-pyridin-4-ylbutanoic acid |
| Bn | Benzyl |
| BnBr | Benzyl bromide |
| Boc | tert-Butoxy-carbonyl |
| Boc₂O | Di-tert-butyl dicarbonate |
| BSA | Bovine serum albumin |
| Bu₂Sn(OAc)₂ | Dibutylstannanediyl diacetate |
| C or Cys | L-Cysteine |
| c3amCb1c | cis-3-(Aminomethyl)cyclobutane-1-carboxylic acid |
| cAcBu | cis-3-Aminocyclobutane-1-carboxylic acid |
| CaCl₂ | Calcium chloride |
| cat. # | Catalog number |
| Cbg | L-Cyclobutylglycine |
| Cbz or Z | Benzyloxycarbonyl |
| CbzCl | Benzyl chloroformate |
| Cbz-OSu | N-(Benzyloxycarbonyloxy)succinimide |
| CD | Crohn's disease |
| CeG | 3-(Carboxymethylamino)propanoic acid |
| CH₂Cl₂ or DCM | Dichloromethane |
| CH₂O | Formaldehyde |
| Chloroform-d | Deuterated chloroform |
| cHxc1acid4NH2 | cis-4-Aminocyclohexane-1-carboxylic acid |
| Cit | L-Citrulline |
| ClAc | Acetyl cross-linker |
| cPrS | (S)-2-Amino-2-(1-hydroxycyclopropyl)acetic acid |
| Cs₂CO₃ | Cesium carbonate |
| CuCl | Copper(I) chloride |
| CuI | Copper(I) iodide |
| Cysteamine | β-Mercaptoethylamine or 2-aminoethanethiol |
| d | Doublet |
| D or Asp | L-Aspartic acid |
| Da | Dalton |
| Dab | L-2,4-Diaminobutyric acid |
| daMeP | (R)-2-Methylpyrrolidine-2-carboxylic acid or α-methyl-D-proline |
| Dap | L-2,3-Diaminopropionic acid |
| DapMe | (S)-2-Amino-3-(methylamino)propanoic acid |
| DapUrea | (S)-2-Amino-3-(carbamoylamino)propanoic acid |
| DAST | Diethylaminosulfur trifluoride |
| dbhcLeu | (1S,3R)-3-aminocyclopentane-1-carboxylic acid |
| DBU | 1,8-Diazabicyclo[5.4.0]undec-7-ene |
| DCC | N,N′-Dicyclohexylcarbodiimide |
| DCE | Dichloroethane |
| DCM or CH₂Cl₂ | Dichloromethane |
| dd | Doublet of doublets |
| DI | Deionized |
| DIAD | Diisopropyl azodicarboxylate |
| DIC | N′,N′-Diisopropylcarbodiimide |
| DIPEA | N,N-Diisopropylethylamine or Hunig's base |
| DMA | N,N-Dimethylacetamide |
| DMAP | 4-Dimethylaminopyridine |
| DMF | N,N-Dimethylformamide |
| DMSO | Dimethylsulfoxide |
| DMSO-d₆ | Hexadeuterodimethyl sulfoxide |
| dNMeA | N-Methyl-D-alanine |
| dNMeE | N-Methyl-D-glutamic acid |
| DODT | 3,6-Dioxa-1,8-octanedithiol |
| dP | D-Proline |
| DPBS | Dulbe’co′s phosphate-buffered saline |
| dPro44F2 | (R)-4,4-Difluoropyrrolidine-2-carboxylic acid |
| dProc4CH2COOH | (2R,4S)-4-(Carboxymethyl)pyrrolidine-2-carboxylic acid |
| dPro4cPr | (R)-5-Azaspiro[2.4]heptane-6-carboxylic acid |
| dProc3OH | (2R,3S)-3-Hydroxypyrrolidine-2-carboxylic acid |
| dProc4CH2COOH | (2R,4S)-4-(Carboxymethyl)pyrrolidine-2-carboxylic acid |
| dProc4NH2 | (2R,4R)-4-Aminopyrrolidine-2-carboxylic acid |
| dProc4NMe2 | (2R,4R)-4-(Dimethylamino)pyrrolidine-2-carboxylic acid |
| dProc4OH | (2R,4R)-4-Hydroxypyrrolidine-2-carboxylic acid |
| dProt4CH2NH2 | (2R,4R)-4-(Aminomethyl)pyrrolidine-2-carboxylic acid |
| dProt4F | (2R,4S)-4-Fluoropyrrolidine-2-carboxylic acid |
| dProt4NH2 | (2R,4S)-4-Aminopyrrolidine-2-carboxylic acid |
| dProt4NHC2acidNMe3 | 2-(((3S,5R)-5-Carboxypyrrolidin-3-yl)amino)-N,N,-trimethyl-2-oxoethan-1-aminium |
| dProt4NMe2 | (2R,4S)-4-(Dimethylamino)pyrrolidine-2-carboxylic acid |
| dProt4NMe3 | (3S,5R)-5-Carboxy-N,N,N-trimethylpyrrolidin-3-aminium |
| dProt4OAcOH | (2R,4S)-4-(Carboxymethoxy)pyrrolidine-2-carboxylic acid |
| dProt4OEtNMe3 | 2-(((3S,5R)-5-Carboxypyrrolidin-3-yl)oxy)-N,N,N-trimethylethan-1-aminium |
| dProt4OH | (2R,4S)-4-Hydroxypyrrolidine-2-carboxylic acid |
| dProt4OMe | (2R,4S)-4-Methoxypyrrolidine-2-carboxylic acid |
| dProt4Tet | (2R,4S)-4-(2H-Tetrazol-5-y1)pyrrolidine-2-carboxylic acid |
| dt | Doublet of triplets |
| DTT | Dithiothreitol |
| DVB | Divinylbenzene |
| E or Glu | L-Glutamic acid |
| EC50 or EC₅₀ | Half maximal effective concentration |
| ECD | Extracellular domain |
| EDC or EDCI | 1-Ethyl-3-(3-dimethylaminopropyl)carbodiimide hydrochloride or N-(3-dimethylaminopropyl)-N′- |
| | ethylcarbodiimide hydrochloride |
| EDTA | Ethylenediaminetetraacetic acid |
| ESI | Electrospray ionization |
| Et₂O | Diethyl ether |
| EtOAc | Ethyl acetate |
| EtSH | Ethanethiol |
| F or Phe | L-Phenylalanine |
| F3MCONH2 | (S)-2-Amino-3-(3-(2-amino-2-oxoethyl)phenyl)propanoic acid |
| F4MCO2H | (S)-2-Amino-3-(4-(carboxymethyl)phenyl)propanoic acid |
| F4mSO3H | (S)-2-Amino-3-[4-(sulfomethyl)phenyl]propanoic acid |
| F4SO2NH2 | (S)-2-Amino-3-(4-sulfamoylphenyl)propanoic acid |
| F4SO3H | (S)-2-Amino-3-(4-sulfophenyl)propanoic acid |
| F4urea | (S)-2-Amino-3-(4-ureidophenyl)propanoic acid |
| FA or HCOOH | Formic acid |
| Fmoc | 9H-Fluoren-9-ylmethoxycarbonyl |
| Fmoc-OSu | N-(9-Fluorenylmethoxycarbonyloxy)succinimide |
| Furan | (S)-2-Amino-3-(furan-2-yl)propanoic acid or L-2-furylalanine |
| FX1 | N⁵-(Pyridin-3-yl)-L-glutamine |
| FX2 | (S)-2-Amino-4-(isoxazole-3-carboxamido)butanoic acid |
| G or Gly | Glycine |
| h | Hour |
| H or His | L-Histidine |
| H₂ | Hydrogen |
| Hag | (S)-2-Aminohex-5-enoic acid |
| HATU | 1-[Bis(dimethylamino)methylene]-1H-1,2,3-triazolo[4,5-b]pyridinium 3-oxid |
| | hexafluorophosphate or N-[(dimethylamino)-1H-1,2,3-triazolo-[4,5-b]pyridin-1- |
| | ylmethylene]-N-methylmethanaminium hexafluoro-phosphate N-oxide |
| HCOOH or FA | Formic acid |
| hE | L-Homoglutamic acid or (S)-2-aminohexanedioic acid |
| HEPES | (4-(2-Hydroxyethyl)-1-piperazineethanesulfonic acid) (zwitterionic sulfonic acid buffering agent) |
| hF | L-Homophenylalanine |
| HFIP | 1,1,1,3,3,3-Hexafluoroisopropanol |
| hhL | (S)-2-Amino-6-methylheptanoic acid |
| hK | L-Homolysine or (S)-2,7-diaminoheptanoic acid |
| hL | L-Homoleucine or (S)-2-amino-5-methylhexanoic acid |
| HOAt | 1-Hydroxy-7-azabenzotriazole |
| HOBt | 1-Hydroxybenzotriazole hydrate |
| HPLC | High-performance liquid chromatography |
| hR | L-Homoarginine or (S)-2-amino-6-carbamimidamidohexanoic acid |
| hS | L-Homoserine or (S)-2-amino-4-hydroxybutanoic acid |
| hSOMe | (S)-2-Amino-4-methoxybutanoic acid |
| Hz | Hertz |
| hν | Energy of a photon |
| I₂ | Iodine |
| IBD | Inflammatory bowel diseases |
| IC50 or IC₅₀ | Half maximal inhibitory concentration |
| Im | Imidazole |
| IPA or i-PrOH | Isopropanol |
| [Ir(cod)(PCy₃)(py)]PF₆ | (1,5-Cyclooctadiene)(pyridine)(tricyclohexylphosphine)-iridium(I) hexafluorophosphate |
| JRA | Juvenile rheumatoid arthritis |
| K or Lys | L-Lysine |
| K_{D} | Equilibrium dissociation constant |
| K_{D}^{app} | Apparent substrate dissociation constant |
| KH₂PO₄ | Monobasic potassium phosphate |
| k_{off} | Dissociation rate constant |
| KOH | Potassium hydroxide |
| kₒₙ | Association rate constant |
| L or Leu | L-Leucine |
| L | Liter |
| LAH | Lithium aluminum hydride |
| LC-MS | Liquid chromatography-mass spectrometry |
| LiHMDS | Lithium bis(trimethylsilyl)amide |
| LiOH | Lithium hydroxide |
| LiOMe | Lithium methoxide |
| LL | Low loading |
| LysMe | (S)-2-Amino-6-(methylamino)hexanoic acid |
| LysMe2 | (S)-2-Amino-6-(dimethylamino)hexanoic acid |
| LysMe3 | [(S)-5-Amino-5-carboxypentyl]-trimethylazanium |
| LysN3 | (S)-2-Amino-6-azidohexanoic acid |
| m | Multiplet |
| M | Molar |
| mAF | (S)-2-Amino-3-(3-(aminomethyl)phenyl)propanoic acid |
| MBHA | 4-Methylbenzhydrylamine |
| mCPBA | 3-Chloroperbenzoic acid |
| Me2AEF | (S)-2-Amino-3-[4-[2-(dimethylamino)ethoxy]phenyl]propanoic acid |
| MeCN | Acetonitrile |
| MeI | Iodomethane |
| MeLi | Methyllithium |
| MeMgCl | Methyl magnesium chloride |
| MeOH | Methanol |
| Methanol-d₄ | Tetradeuteromethanol |
| MHz | Megahertz |
| min | Minute |
| mL | Milliliter |
| mm | Millimeter |
| mM | Millimolar |
| mmol | Millimole |
| Mn | Manganese |
| MO2 | L-Methionine sulfone or (S)-2-amino-4-(methylsulfonyl) butanoic acid |
| MOA | Mechanism of action |
| mol | Mole |
| Morpip6diMe | (S)-6,6-Dimethylmorpholine-3-carboxylic acid |
| MPLC | Medium-pressure liquid chromatography |
| MS | Mass spectrometry |
| MTBE or TBME | tert-Butyl methyl ether |
| N or Asn | L-Asparagine |
| N | Normality |
| N₂ | Nitrogen |
| N2SPrOHG | (S)-(1-Hydroxypropan-2-yl)glycine |
| n-BuLi | n-Butyllithium |
| NA | Not applicable |
| Na₂CO₃ | Sodium carbonate |
| NaClO₂ | Sodium chlorite |
| Na₂SO₄ | Sodium sulfate |
| NaBH(OAc)₃ | Sodium triacetoxyborohydride |
| NaClO | Sodium hypochlorite |
| NaClO₂ | Sodium chlorite |
| NaH | Sodium hydride |
| NaHCO₃ | Sodium bicarbonate |
| NaHMDS | Sodium bis(trimethylsilyl)amide |
| NaI | Sodium iodide |
| NaIO₄ | Sodium periodate |
| NaOH | Sodium hydroxyde |
| NBS | N-Bromosuccinimide |
| NCS | N-Chlorosuccinimide |
| NEt3OxadG | (2-(1,3,4-Oxadiazol-2-yl)ethyl)glycine |
| NEtAla | N-Ethyl-L-alanine |
| NEtCNG | 2-(2-Cyanoethylamino)acetic acid or (2-cyanoethyl)glycine |
| NEtCONH2A | (3-Amino-3-oxopropyl)-L-alanine |
| NEtCONH2G | 2-[(3-Amino-3-oxopropyl)amino]acetic acid or (3-amino-3-oxopropyl)glycine |
| NEtdA | N-Ethyl-D-alanine |
| NEtG | 2-(Ethylamino)acetic acid or 2-(ethylamino)glycine |
| NF-κB | Nuclear factor kappa-light-chain-enhancer of activated B cells |
| NH2 or NH₂ | C-Terminal amide |
| NH₄Cl | Ammonium chloride |
| NH₄OH | Ammonium hydroxide |
| NhEG | 4-(Carboxymethylamino)butanoic acid |
| NiBr₂•glyme | Nickel(II) bromide ethylene glycol dimethyl ether complex |
| NiCl₂•glyme | Nickel(II) chloride ethylene glycol dimethyl ether complex |
| NIS | N-Iodosuccinimide |
| Nle | L-Norleucine |
| Nle3F2 | (S)-2-Amino-5,5-difluorohexanoic acid |
| nM | Nanomolar |
| nm | Nanometer |
| NMeA | N-Methyl-L-alanine |
| NMeDab | (S)-4-Amino-2-(methylamino)butanoic acid |
| NMeE | N-Methyl-L-glutamic acid |
| NMehSMe | (S)-4-Methoxy-2-(methylamino)butanoic acid |
| NMeN | N-Methyl-L-asparagine |
| NMeS | N-Methyl-L-serine |
| NMeT | N-Methyl-L-threonine |
| NMM | N-Methylmorpholine |
| NMO | 4-Methylmorpholine N-oxide |
| NMP | N-Methyl-2-pyrrolidone |
| NMR | Nuclear magnetic resonance |
| NxG21 | 2-(2-Hydroxyethylamino)acetic acid |
| NxG27 | 2-(3-Methoxypropylamino)acetic acid |
| NxG29 | 2-(2-Methoxyethylamino)acetic acid |
| NxG38 | 2-(3-Hydroxypropylamino)acetic acid |
| NxG41 | 2-[(3-Hydroxy-2,2-dimethylpropyl)amino]acetic acid |
| NxG42 | 2-[(1-Hydroxycyclopropyl)methylamino]acetic acid |
| o.n. | Overnight |
| OrnN3 | (S)-2-Amino-5-azidopentanoic acid |
| OsO₄ | Osmium tetroxide |
| OSu | O-Succinimide |
| Oxyma Pure | Ethyl cyano(hydroxyimino)acetate |
| P or Pro | L-Proline |
| P20 | Polysorbate 20 |
| Pbf | 2,2,4,6,7-Pentamethyldihydrobenzofuran-5-sulfonyl |
| PBS | Phosphate-buffered saline |
| Pd/C | Palladium on carbon |
| Pd(PPh₃)₄ | Tetrakis(triphenylphosphine)palladium(0) |
| PE | Petroleum ether |
| PfTrpB-0B2 | Engineered tryptophan synthase from Pyrococcus furiosus |
| PfTrpB-7E6 | Engineered tryptophan synthase from Pyrococcus furiosus |
| PG | Protecting group |
| PhCF₃ | α,α,α-Trifluorotoluene |
| Phe2F4COOH | (S)-4-(2-Amino-2-carboxyethyl)-3-fluorobenzoic acid |
| Phe3Cl4COOH | (S)-4-(2-Amino-2-carboxyethyl)-2-chlorobenzoic acid |
| Phe3CONH2 | (S)-2-Amino-3-(3-carbamoylphenyl)propanoic acid |
| Phe3COOH | (S)-3-(2-Amino-2-carboxyethyl)benzoic acid |
| Phe3F4COOH | (S)-4-(2-Amino-2-carboxyethyl)-2-fluorobenzoic acid |
| Phe35F24COOH | (S)-4-(2-Amino-2-carboxyethyl)-2,6-difluorobenzoic acid |
| Phe4AcPip | (S)-3-[4-(4-Acetylpiperazin-1-yl)phenyl]-2-aminopropanoic acid |
| Phe4CH2NH2 | (S)-2-Amino-3-(4-(aminomethyl)phenyl)propanoic acid |
| Phe4CONH2 | (S)-2-Amino-3-(4-carbamoylphenyl)propanoic acid |
| Phe4COOH | L-Phenylalanine-4-carboxylic acid or (S)-4-(2-amino-2-carboxyethyl)benzoic acid |
| Phe4COOMe | (S)-2-Amino-3-(4-methoxycarbonylphenyl)propanoic acid |
| PhMgBr | Phenylmagnesium bromide |
| PhMe₂SiH | Dimethylphenylsilane |
| PhSiH₃ | Phenylsilane |
| PhOH | Phenol |
| Pin | Pinacolato |
| (Pin)₂B₂ | Bis(pinacolato)diboron |
| Pip | (S)-Piperidine-2-carboxylic acid |
| Pip2c | (S)-Piperazine-2-carboxylic acid |
| Pipc4NH2 | (2S,4R)-4-Aminopiperidine-2-carboxylic acid |
| Pipc4OH | (2S,4R)-4-Hydroxypiperidine-2-carboxylic acid |
| Pipc5CO2H | (2S,5S)-Piperidine-2,5-dicarboxylic acid |
| Pipc5OH | (2S,5S)-5-Hydroxypiperidine-2-carboxylic acid |
| Pipt5Me | (2S,5R)-5-Methylpiperidine-2-carboxylic acid |
| Pipt5OH | (2S,5R)-5-Hydroxypiperidine-2-carboxylic acid |
| PLP | 3-Hydroxy-2-methyl-5-([phosphonooxy]methyl)-4-pyridinecarboxaldehyde or pyridoxal 5-phosphate |
| POCl₃ | Phosphorus(V) oxychloride |
| PPh₃ | Triphenylphosphine |
| ppm | Part per million |
| Prot4NH2 | (2S,4R)-4-Aminopyrrolidine-2-carboxylic acid |
| psi | Pound-force per square inch |
| PyAOP | (7-Azabenzotriazol-1-yloxy)tripyrrolidinophosphonium hexafluorophosphate |
| PyrimAla | (S)-2-Amino-3-(pyrimidin-5-yl)propanoic acid |
| q | Quadruplet |
| Q or Gln | L-Glutamine |
| Qdm | N⁵,N⁵-Dimethyl-L-glutamine |
| R or Arg | L-Arginine |
| RA | Rheumatoid arthritis |
| RbMe4Pal | (2S,3R)-2-Amino-3-pyridin-4-ylbutanoic acid |
| RbMeY | (2S,3R)-2-Amino-3-(4-hydroxyphenyl)butanoic acid |
| RbOH4Pal | (2S,3R)-2-Amino-3-hydroxy-3-pyridin-4-ylpropanoic acid |
| RbOHAsn | (2S,3R)-2,4-Diamino-3-hydroxy-4-oxobutanoic acid |
| RbOHPhe | (2S,3R)-2-Amino-3-hydroxy-3-phenylpropanoic acid |
| RbOHPhe4CONH2 | (2S,3R)-2-Amino-3-(4-carbamoylphenyl)-3-hydroxypropanoic acid |
| RbOHPhe4COOH | 4-((1R,2S)-2-Amino-2-carboxy-1-hydroxyethyl)benzoic acid or (2S,3R)-2-amino-3-(4- |
| | carboxyphenyl)-3-hydroxypropanoic acid |
| RP or Rp | Reversed phase |
| rpm | Revolutions per minute |
| RPMI | Roswell Park Memorial Institute 1640 medium |
| RT | Room temperature |
| RU | Response unit |
| RuCl₃ | Ruthenium(III) chloride |
| S or Ser | L-Serine |
| Sar | Sarcosine or N-methylglycine |
| sat. | Saturated |
| SbMe4Pal | (2S,3S)-2-Amino-3-pyridin-4-ylbutanoic acid |
| SbMePhe4COOH | 4-((1S,2S)-1-Amino-1-carboxypropan-2-yl)benzoic acid |
| SbMeW | (2S,3S)-2-Amino-3-(1H-indol-3-yl)butanoic acid |
| SbMeW2Me | (2S,3S)-2-Amino-3-(2-methyl-1H-indol-3-yl)butanoic acid |
| SbMeW2Me7OMe | (2S,3S)-2-Amino-3-(7-methoxy-2-methyl-1H-indol-3- |
| | yl)butanoic acid |
| SbMeW4OMe | (2S,3S)-2-Amino-3-(4-methoxy-1H-indol-3-yl)butanoic acid |
| SbMeW7Me | (2S,3S)-2-Amino-3-(7-methyl-1H-indol-3-yl)butanoic acid |
| SbMeW7OMe | (2S,3S)-2-Amino-3-(7-methoxy-1H-indol-3-yl)butanoic acid |
| SbMeY | (2S,3S)-2-Amino-3-(4-hydroxyphenyl)butanoic acid |
| SbOHAsn | (2S,3S)-2,4-Diamino-3-hydroxy-4-oxobutanoic acid |
| SbOHPhe4CONH2 | (2S,3S)-2-Amino-3-(4-carbamoylphenyl)-3-hydroxypropanoic acid |
| SbPrW | (2S,3S)-2-Amino-3-(1H-indol-3-yl)hexanoic acid |
| SBrPh | (S)-2-Phenylacetyl cross-linker |
| SerOEt | O-Ethyl-L-serine or (S)-2-amino-3-ethoxypropanoic acid |
| SerOCH2cPr | O-(Cyclopropylmethyl)-L-serine or (S)-2-amino-3-cyclopropylmethoxypropanoic acid |
| SerOC2NH2 | O-(2-Aminoethyl)-L-serine |
| SFC | Supercritical fluid chromatography |
| SOC | Standard of care |
| SOCl₂ | Thionyl chloride |
| SPip | (R)-thiomorpholine-3-carboxylic acid |
| SPPS | Solid-phase peptide synthesis |
| SPR | Surface plasmon resonance |
| sTNFR1 | Soluble human TNFR1 |
| t | Triplet |
| T or Thr | L-Threonine |
| TBAB | Tetrabutylammonium bromide |
| TBACN | Tetrabutylammonium cyanide |
| TBAI | Tetrabutylammonium iodide |
| TBS or TBDMS | tert-Butyldimethylsilyl |
| TBSCl or TBDMSCl | tert-Butyldimethylsilyl chloride |
| TBSOTf | tert-Butyldimethylsilyl trifluoromethanesulfonate |
| TBTA | tert-Butyl 2,2,2-trichloroacetimidate |
| tBu or tBu or t-Bu | tert-Butyl |
| t-BuOH | tert-Butanol |
| TEA | Triethylamine |
| TEMPO | 2,2,6,6-Tetramethylpiperidinyloxy |
| TESCl | Chlorotriethylsilane or trimethylsilyl chloride |
| Tf₂NH | Bistriflimide or bis(trifluoromethane)sulfonimide |
| TFA | Trifluoroacetic acid |
| THF | Tetrahydrofuran |
| TIS | Triisopropylsilane |
| TLC | Thin layer chromatography |
| TMSCl | Chlorotrimethylsilane |
| TMSN₃ | Azidotrimethylsilane |
| TNFR1 | Tumor necrosis factor receptor 1 or CD120a |
| TNFR2 | Tumor necrosis factor receptor 2 or CD120b |
| TNFα | Tumor necrosis factor-alpha |
| TR-FRET | Time-Resolved Fluorescence Resonance Energy Transfer |
| Trp1Me | 1-Methyl- L-tryptophan |
| Trp4Cl | (S)-2-Amino-3-(4-chloro-1H-indol-3-yl)propanoic acid |
| Trp6F7Cl | (S)-2-Amino-3-(7-chloro-6-fluoro-1H-indol-3-yl)propanoic acid |
| Trp7az | (S)-2-Amino-3-(1H-pyrrolo[2,3-b]pyridin-3-yl)propanoic acid |
| Trp7azCH2COOH | (S)-2-Amino-3-[1-(carboxymethyl)pyrrolo[2,3-b]pyridin-3-yl]propanoic acid |
| Trp7Br | (S)-2-Amino-3-(7-bromo-1H-indol-3-yl)propanoic acid |
| Trp7Cl | (S)-2-Amino-3-(7-chloro-1H-indol-3-yl)propanoic acid |
| Trp7CN | (S)-2-Amino-3-(7-cyano-1H-indol-3-yl)propanoic acid |
| Trp7F | (S)-2-Amino-3-(7-fluoro-1H-indol-3-yl)propanoic acid |
| TrpCH2COOH | (S)-2-Amino-3-[1-(carboxymethyl)indol-3-yl]propanoic acid or or 1-(carboxymethyl)-L-tryptophan |
| Trt | Trityl or triphenylmethyl |
| TrtCl | Triphenylmethyl chloride |
| Trt-OH | Triphenylmethanol or trityl alcohol |
| TrtNC | Trityl isocyanide |
| TsCl | p-Toluenesulfonyl chloride |
| TsOH | p-Toluenesulfonic acid monohydrate |
| Tyr2F | (S)-2-Amino-3-(2-fluoro-4-hydroxyphenyl)propanoic acid |
| Tyr3OMe | (S)-2-Amino-3-(4-hydroxy-3-methoxyphenyl)propanoic acid |
| TyrOCF3 | (S)-2-Amino-3-[4-(trifluoromethoxy)phenyl]propanoic acid |
| TyrOcm | (S)-2-Amino-3-(4-(carboxymethoxy)phenyl)propanoic acid |
| TyrOMe | (S)-2-Amino-3-(4-methoxyphenyl)propanoic acid |
| Tza | (S)-2-Amino-3-(1,3-thiazol-4-yl)propanoic acid |
| TZAla | (S)-2-Amino-3-(1,3-thiazol-5-yl)propanoic acid |
| UC | Ulcerative colitis |
| UHP | Urea hydrogen peroxide |
| uM or μM | Micromolar |
| UPLC | Ultra performance liquid chromatography or ultrahigh pressure liquid chromatography |
| UV | Ultraviolet |
| V | Volume |
| v/v | Volume per volume |
| W or Trp | L-Tryptophan |
| w/w | Weight of compound to total weight |
| XPhos Pd G2 | Chloro(2-dicyclohexylphosphino-2′,4′,6′-triisopropyl-1,1′-biphenyl)[2-(2′- |
| | amino-1,1′-biphenyl)]palladium(II) |
| Y or Tyr | L-Tyrosine |
| Zn* | Activated zinc |
| ZnCl₂ | Zinc chloride |

### Reaction Schemes

### Intermediates and Monomers Syntheses

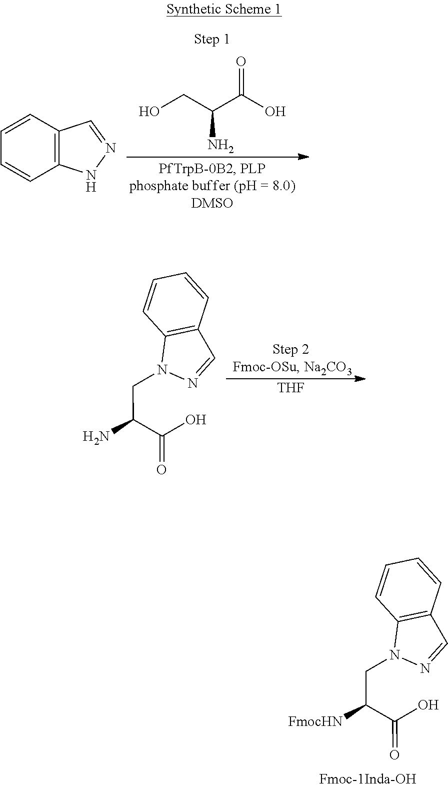

### (S)-2-((9H-Fluoren-9-yl)methoxy)carbonyl)amino)-3-(1H-indazol-1-yl)propanoic acid (Fmoc-1Inda-OH)

Step 1: To a mixture of indazole (110 g, 931 mmol) and L-serine (97.9 g, 931 mmol) in DMSO (1.1 L) and 0.2 M potassium phosphate buffer (4.4 L, pH ˜8.0) under a nitrogen atmosphere were added PLP (3.05 g, 9.31 mmol) and PfTrpB-0B2 (7.7 g, 7 wt. %). The mixture was stirred and heated at 65° C. for 18 h. Upon completion, the resulting solution was used directly in the next step.

Step 2: The mixture containing (2S)-2-amino-3-(indazol-1-yl)propanoic acid (191 g in assay yield) was diluted with tetrahydrofuran (2.1 L). Na₂CO₃ (197 g, 1.86 mol) and Fmoc-OSu (314 g, 931 mmol) were added to the mixture at 15° C. under a nitrogen atmosphere. The mixture was stirred at RT for 18 h under a nitrogen atmosphere. The mixture was acidified to pH ˜3 with 6 N HCl. The mixture was filtered, and the filtrate was extracted with EtOAc (3×3.0 L). The organic layers were combined, washed with H₂O (2×2.0 L) and brine (3.0 L), dried over anh. Na₂SO₄, filtered, and concentrated under reduced pressure. The residue was triturated with DCM/Hexane (5 L/2.5 L) four times. The solids were collected by filtration and dried under reduced pressure to afford (2S)-2-{[(9H-fluoren-9-ylmethoxy)carbonyl]amino}-3-(indazol-1-yl)propanoic acid. C₂₅H₂₂N₃O₄[M+H]⁺ 428.2, found 428.2. ¹H NMR (300 MHz, DMSO-d₆): δ 13.11 (s, 1H), 8.10 (s, 1H), 7.97-7.89 (m, 2H), 7.79-7.69 (m, 3H), 7.60-7.52 (m, 2H), 7.47-7.21 (m, 5H), 7.14-7.09 (m, 1H), 4.77-4.60 (m, 2H), 4.57-4.52 (m, 1H), 4.29-4.10 (m, 3H).

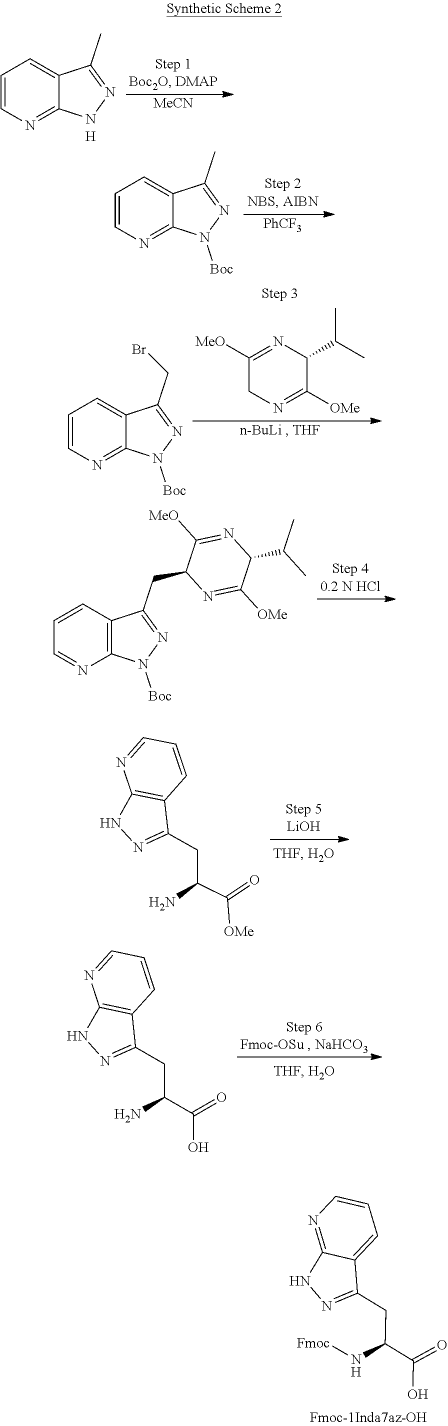

### (S)-2-((((9H-Fluoren-9-yl)methoxy)carbonyl)amino)-3-(1H-pyrazolo[3,4-b]pyridin-3-yl)propanoic acid (Fmoc-1Inda7az-OH)

Step 1: DMAP (0.46 g, 3.8 mmol) and Boc₂O (10.4 mL, 45.1 mmol) under Ar at RT were added to a solution of 3-methyl-1H-pyrazolo[3,4-b]pyridine (5.0 g, 37.6 mmol) in MeCN (100 mL. The reaction was stirred over night at RT, then concentrated in vacuum. The residue was purified by silica gel column chromatography, eluting with EtOAc 0-40% in PE to give tert-butyl 3-methyl-1H-pyrazolo[3,4-b]pyridine-1-carboxylate. MS ESI calculated for C₂H₁₆N₃O₂ [M+H]⁺ 234.12, found 234.05. ¹H NMR (300 MHz, chloroform-d): δ 8.74-8.73 (m, 1H), 8.03-8.01 (m, 1H), 7.2-7.28 (m, 1H), 2.61 (s, 3H), 1.73 (s, 9H).

Step 2: AIBN (0.53 g, 3.22 mmol) and NBS (5.72 g, 32.2 mmol) under Ar at RT were added to a solution of tert-butyl 3-methyl-1H-pyrazolo[3,4-b]pyridine-1-carboxylate (7.5 g, 32.2 mmol) in trifluorotoluene (170 mL). The reaction was stirred at 80° C. for 1 h then cooled to RT and concentrated in vacuum. The residue was purified by silica gel column chromatography, eluting with EtOAc 0-25% in PE to give tert-butyl 3-(bromomethyl)-1H-pyrazolo[3,4-b]pyridine-1-carboxylate. MS ESI calculated for C₁₂H₁₅BrN₃O₂[M+H]⁺ 312.03/314.03, found 311.90/313.90. ¹H NMR (300 MHz, chloroform-d): δ 8.79-8.78 (m, 1H), 8.29-8.20 (m, 1H), 7.37-7.35 (m, 1H), 4.77 (s, 2H), 1.73 (s, 9H).

Step 3: n-BuLi (8.6 mL, 21.5 mmol, 2.5 N in hexane) was added dropwise over 30 min at −78° C. under Ar atmosphere to a solution of tert-butyl 3-(bromomethyl)-1H-pyrazolo[3,4-b]pyridine-1-carboxylate (6.4 g, 20.5 mmol) in anh. THF (150 mL). Then (R)-2-isopropyl-3,6-dimethoxy-2,5-dihydropyrazine (3.8 g, 20.5 mmol) in anh. THF (10 mL) was added. The resulting mixture was stirred at −78° C. for 4 h. The reaction mixture was quenched with aq. sat. NH₄Cl (200 mL) at 0° C. and extracted with EtOAc (2×500 mL). The combined organic layers were washed with brine (2×300 mL), dried over anh. Na₂SO₄, filtered, and concentrated. The residue was purified by silica gel column chromatography, eluting with EtOAc 0-50% in PE to afford tert-butyl 3-(((2S,5R)-5-isopropyl-3,6-dimethoxy-2,5-dihydropyrazin-2-yl)methyl)-1H-pyrazolo[3,4-b]pyridine-1-carboxylate. MS ESI calculated for C₂₁H₃₀N₅O₄ [M+H]⁺ 416.22, found 416.05. ¹H NMR (300 MHz, chloroform-d): δ 8.71-8.70 (m, 1H), 8.13-7.98 (m, 1H), 7.33-7.19 (m, 1H), 4.45 (s, 1H), 3.70 (s, 3H), 3.57-3.55 (m, 4H), 3.35-3.33 (m, 1H), 2.18 (s, 1H), 1.71 (s, 10H), 0.98 (d, J=6.9 Hz, 3H), 0.66 (d, J=6.9 Hz, 3H).

Step 4: 0.2 N HCl (81.8 mL, 16.36 mmol) at 0° C. was added to a stirred solution of tert-butyl 3-(((2S,5R)-5-isopropyl-3,6-dimethoxy-2,5-dihydropyrazin-2-yl)methyl)-1H-pyrazolo[3,4-b]pyridine-1-carboxylate (3.4 g, 8.18 mmol) in MeCN (50 mL). The resulting mixture was stirred at RT for 4 h, then concentrated under reduced pressure to afford methyl (S)-2-amino-3-(1H-pyrazolo[3,4-b]pyridin-3-yl)propanoate, which was used in the next step without purification. MS ESI calculated for C₁₀H₁₃N₄O₂ [M+H]⁺ 221.10, found 221.10.

Step 5: An aq. 1 N LiOH solution (16.4 mL, 16.4 mmol) at RT was added to a solution of methyl (S)-2-amino-3-(1H-pyrazolo[3,4-b]pyridin-3-yl)propanoate (1.8 g, 8.2 mmol) in THF (16.4 mL). The mixture was stirred at RT for 2 h then acidified with 1 N HCl. The resulting solution was concentrated under reduced pressure to afford (S)-2-amino-3-(1H-pyrazolo[3,4-b]pyridin-3-yl)propanoic acid, which was used in the next step without purification. MS ESI calculated for C₉H₁₁N₄O₂[M+H]⁺ 207.08, found 207.10.

Step 6: NaHCO₃(1.9 g, 22.3 mmol) and Fmoc-OSu (5.5 g, 16.3 mmol) at RT were added to a stirred solution of (S)-2-amino-3-(1H-pyrazolo[3,4-b]pyridin-3-yl)propanoic acid (1.7 g, 7.4 mmol) in THF (30 mL) and water (30 mL). After the resulting mixture was stirred o.n. (over night) at RT, the pH was adjusted to ˜3 with 1 N HCl and the mixture was extracted with EtOAc (3×200 mL). The combined organic layers were washed with brine (2×50 mL), dried over anh. Na₂SO₄, filtered, and concentrated under reduced pressure. The residue was recrystallized from MeCN (50 mL) to give (S)-2-((((9H-fluoren-9-yl)methoxy)carbonyl)amino)-3-(1H-pyrazolo[3,4-b]pyridin-3-yl)propanoic acid. MS ESI calculated for C₂₄H₂₁N₄O₄[M+H]⁺ 429.15, found 429.05. ¹H NMR (400 MHz, DMSO-d₆): δ 13.52-12.70 (m, 2H), 8.48-8.47 (m, 1H), 8.30-8.28 (m, 1H), 7.88-7.86 (m, 3H), 7.63-7.61 (m, 2H), 7.42-7.40 (m, 2H), 7.38-7.35 (m, 2H), 7.12-7.10 (m, 1H), 4.47-4.46 (m, 1H), 4.21-4.10 (m, 3H), 3.41-3.28 (m, 2H).

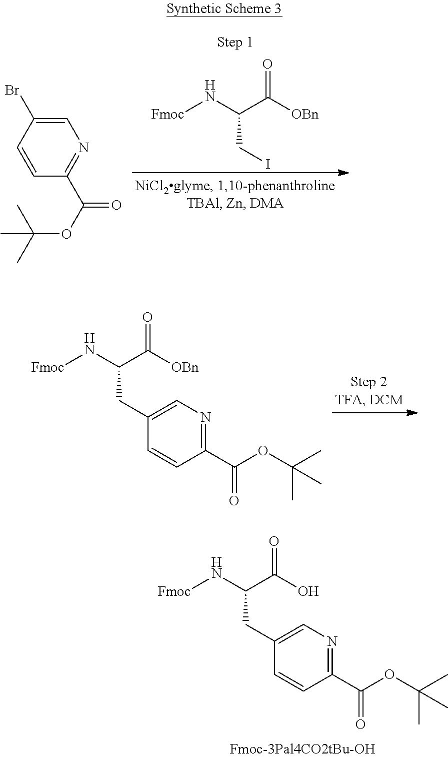

### (S)-2-((((9H-Fluoren-9-yl)methoxy)carbonyl)amino)-3-(6-(tert-butoxycarbonyl)pyridin-3-yl)propanoic acid (Fmoc-3Pal4CO2tBu-OH)

Step 1: A mixture of NiCl₂·glyme (710 mg, 3.2 mmol) and 1,10-phenanthroline (700 mg, 3.2 mmol) in anh. DMA (160 mL) was stirred and heated to 50° C. under a nitrogen atmosphere for 1 h. The mixture was cooled to RT. tert-Butyl 5-bromopicolinate (4.17 g, 16.2 mmol), benzyl (R)-2-((((9H-fluoren-9-yl)methoxy)carbonyl)amino)-3-iodopropanoate (8.51 g, 16.2 mmol), TBAI (6.19 g, 16.2 mmol), and activated zinc (2.11 g, 32.3 mmol) were added to the mixture. The mixture was stirred at RT for an additional 2 h, and then quenched with H₂O (200 mL) and extracted with EtOAc (2×500 mL). The combined organic phases were washed with brine (3×200 mL), dried over anh. Na₂SO₄ and filtered. The filtrate was concentrated under reduced pressure and the residue was purified by silica gel column chromatography, eluting with EtOAc 0-20% in PE to afford tert-butyl (S)-5-(2-((((9H-fluoren-9-yl)methoxy)carbonyl)amino)-3-(benzyloxy)-3-oxopropyl)picolinate. MS ESI calculated for C₃₅H₃₅N₂O₆ [M+H]⁺ 579.24, found 579.40.

Step 2: tert-Butyl (S)-5-(2-((((9H-fluoren-9-yl)methoxy)carbonyl)amino)-3-(benzyloxy)-3-oxopropyl)picolinate (7.5 g, 13 mmol) was dissolved in EtOAc (75 mL). The flask was evacuated and refilled with N₂ (5 times). Pd/C (1.38 g, 13 mmol, dry, 10 wt. %) was added into the flask. The flask was evacuated and backfilled with H₂ (5 times). The resulting mixture was stirred for 4 h at RT under an atmosphere of dihydrogen. The flask was evacuated and refilled with N₂ (5 times). The resulting mixture was filtered through a Celite® pad. The filtered cake was washed with EtOAc (2×25 mL). The filtrate was concentrated under reduced pressure. The residue was purified by Rp-flash column chromatography with the following conditions: Column: Flash C18 (330 g); Mobile Phase A: water (0.1% TFA), Mobile Phase B: MeCN (0.1% TFA); Gradient Elution: 5-95%; Detector: UV 215 nm. The fractions containing the product were concentrated under reduced pressure to afford (S)-2-((((9H-fluoren-9-yl)methoxy)carbonyl) amino)-3-(6-(tert-butoxycarbonyl)pyridin-3-yl)propanoic acid. MS ESI calculated for C₂₈H₂₉N₂O₆[M+H]⁺ 489.19, found 489.20; ¹H NMR (300 MHz, methanol-d₄): δ 8.57 (s, 1H), 8.03-7.91 (m, 1H), 7.86-7.74 (m, 3H) 7.54-7.53 (m, 2H), 7.36-7.28 (m, 4H), 4.51-4.40 (m, 1H), 4.32-4.13 (m, 3H), 3.39-3.33 (m, 1H), 3.06-3.05 (m, 1H), 1.65-1.59 (m, 9H).

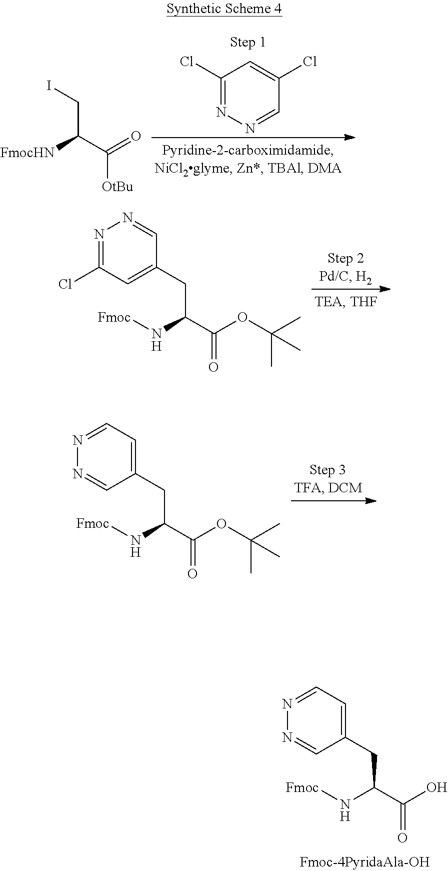

### (S)-2-((((9H-Fluoren-9-yl)methoxy)carbonyl)amino)-3-(pyridazin-4-yl)propanoic acid (Fmoc-4PyridaAla-OH)

Step 1: Pyridine-2-carboximidamide hydrochloride (3.58 g, 22.70 mmol) at RT and under Ar was added to a stirred solution of nickel (II) chloride ethylene glycol dimethyl ether complex (2.49 g, 11.35 mmol) in anh. DMA (200 mL). The solution was stirred at 50° C. for 40 min. The solution was cooled to RT. A solution of TBAI (20.96 g, 56.8 mmol), tert-butyl (R)-2-((((9H-fluoren-9-yl)methoxy)carbonyl)amino)-3-iodopropanoate (28 g, 56.8 mmol), and 3,5-dichloropyridazine (16.91 g, 114 mmol) in anh. DMA (200 mL) was added to the reaction mixture. Activated zinc (7.42 g, 114 mmol) was then added, and the mixture was stirred o.n. at RT. The mixture was diluted with EtOAc (1 L), washed with brine (3×500 mL). The organic layer was dried over anh. Na₂SO₄ and filtered. The filtrate was concentrated under reduced pressure and the residue was purified by a silica gel column chromatography, eluting with EtOAc 0-35% in PE to give tert-butyl (S)-2-((((9H-fluoren-9-yl)methoxy)carbonyl)amino)-3-(6-chloropyridazin-4-yl)propanoate. MS ESI calculated for C₂₆H₂₇ClN₃O₄[M+H]⁺ 480.16, found 480.10. ¹H NMR (400 MHz, chloroform-d): δ 8.96 (s, 1H), 7.80 (d, J=7.6 Hz, 2H), 7.60 (d, J=7.5 Hz, 2H), 7.46-7.29 (m, 5H), 5.49 (s, 1H), 4.63-4.36 (m, 3H), 4.24 (t, J=6.4 Hz, 1H), 3.14-3.12 (m, 2H), 1.45 (s, 9H).

Step 2: tert-Butyl (S)-2-((((9H-fluoren-9-yl)methoxy)carbonyl)amino)-3-(6-chloropyridazin-4-yl)propanoate (20 g, 41.7 mmol) and TEA (16.87 g, 167 mmol) were dissolved in THF (400 mL). The flask was evacuated and refilled with N₂ (5 times). Pd/C (4.43 g, 4.17 mmol, dry, 10 wt. %) was added into the flask. The flask was evacuated and backfilled with H₂ (5 times). The resulting mixture was stirred o.n. at RT under an atmosphere of dihydrogen. The flask was evacuated and refilled with N₂ (5 times). The resulting mixture was filtered through a Celite® pad. The filtered cake was washed with THF (2×50 mL). The filtrate was concentrated under reduced pressure. The residue was purified by a silica gel column chromatography, eluting with EtOAc 0-50% in PE to afford tert-butyl (S)-2-((((9H-fluoren-9-yl)methoxy)carbonyl)amino)-3-(pyridazin-4-yl)propanoate. MS ESI calculated for C₂₆H₂₈N₃O₄ [M+H]⁺ 446.20, found 446.10.

Step 3: To a stirred solution of tert-butyl (S)-2-((((9H-fluoren-9-yl)methoxy)carbonyl) amino)-3-(pyridazin-4-yl)propanoate (19 g, 42.6 mmol) in DCM (140 mL) was added TFA (300 mL) at RT. The solution was stirred at RT for 6 h. The solvent was concentrated under reduced pressure. The residue was recrystallized from MeCN (200 mL) to give (S)-2-((((9H-fluoren-9-yl)methoxy)carbonyl)amino)-3-(pyridazin-4-yl)propanoic acid. MS ESI calculated for C₂₂H₂₀N₃O₄ [M+H]⁺ 390.14, found 390.10. ¹H NMR (400 MHz, DMSO-d₆): δ 12.92 (s, 1H), 9.19-9.11 (m, 2H), 7.89-7.83 (m, 3H), 7.63-7.60 (m, 3H), 7.43-7.28 (m, 4H), 4.34-4.18 (m, 4H), 3.22-3.18 (m, 1H), 2.97-2.91 (m, 1H).

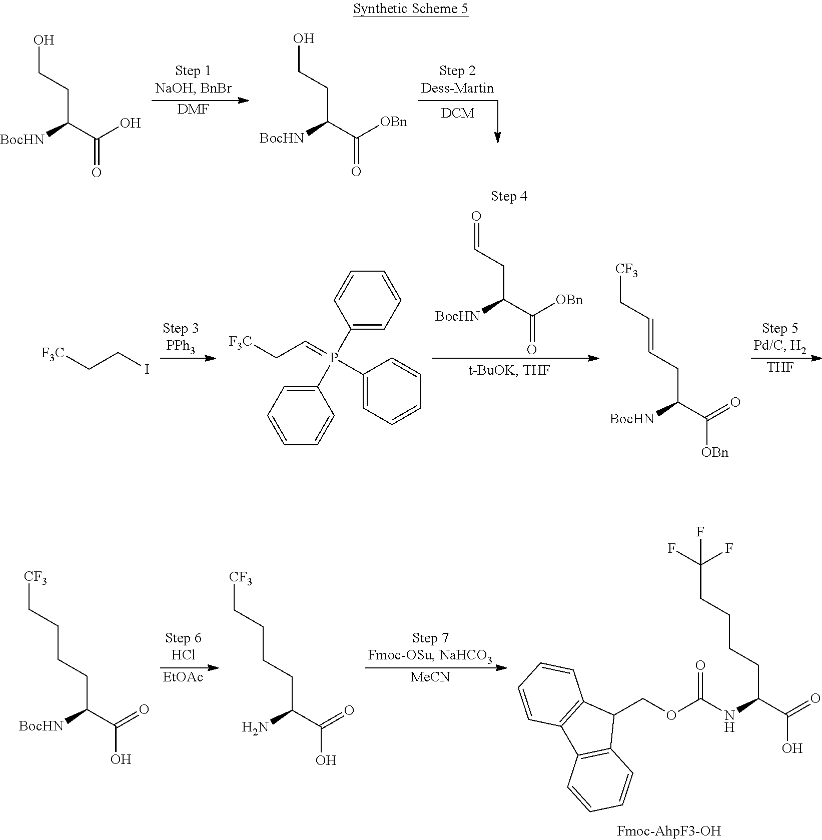

### (S)-2-((((9H-Fluoren-9-yl)methoxy)carbonyl)amino)-7,7,7-trifluoroheptanoic acid (Fmoc-AhpF3-OH)

Step 1: NaOH (3.72 g, 93.0 mmol) at RT was added to a stirred solution of (tert-butoxycarbonyl)-L-homoserine (20.0 g, 91.2 mmol) in EtOH (60 mL) and DI water (60 mL). After 30 min, the reaction mixture was concentrated in vacuo. The residue was dissolved in anh. DMF (60 mL). BnBr (11.1 mL, 93.0 mmol) was added to the solution. The reaction mixture was stirred at RT for 13 h. The reaction mixture was slowly poured into DI water (300 mL) and extracted with EtOAc (2×100 mL). The combined organic layers were washed with brine (50 mL), dried over anh. Na₂SO₄, filtered, and concentrated in vacuo. The residue was purified by silica gel column chromatography, eluting with EtOAc 5-40% in PE to give benzyl (tert-butoxycarbonyl)-L-homoserinate. MS ESI calculated for C₁₆H₂₃NO₅ [M+Na]⁺ 332.15, found 332.0. ¹H NMR (400 MHz, chloroform-d): δ 7.39-7.32 (m, 5H), 5.41-5.39 (m, 1H), 5.23-5.16 (m, 2H), 4.56-4.51 (m, 1H), 3.75-3.61 (m, 2H), 2.20-2.14 (m, 1H), 1.66-1.59 (m, 1H), 1.45 (s, 9H).

Step 2: Dess-Martin periodinane (31.1 g, 73.3 mmol) at RT was added to a stirred solution of benzyl (tert-butoxycarbonyl)-L-homoserinate (20.6 g, 64.6 mmol) in DCM (120 mL). After 30 min, the reaction mixture was quenched by addition of a 1:1 (v/v) NaHCO₃/Na₂S₂O₃ solution (500 mL). The resulting mixture was stirred at RT for 30 min and extracted with DCM (200 mL). The organic layer was washed with brine (50 mL), dried over anh. Na₂SO₄, filtered, and concentrated in vacuo to give benzyl (S)-2-((tert-butoxycarbonyl)amino)-4-oxobutanoate. The crude product was used directly in the next step without further purification. ¹H NMR (400 MHz, chloroform-d): δ 9.72 (s, 1H), 7.39-7.32 (m, 5H), 5.42-5.40 (m, 1H), 5.18 (s, 2H), 4.65-4.63 (m, 1H), 3.14-2.99 (m, 2H), 1.43 (s, 9H).

Step 3: 1,1,1-Trifluoro-3-iodopropane (15.7 mL, 30.0 g, 134 mmol) and PPh₃ (33.4 g, 127 mmol) were stirred o.n. at 80° C. The reaction mixture was cooled down at RT and triturated with PE (80 mL) at RT for 2 h. The solids were recovered by filtration and dried under reduced pressure to afford triphenyl(3,3,3-trifluoropropylidene)-λ⁵-phosphane. ¹H NMR (400 MHz, chloroform-d): δ 7.90-7.86 (m, 9H), 7.85-7.75 (m, 6H), 4.10-4.03 (m, 2H), 2.71-2.57 (m, 3H).

Step 4: A solution of potassium tert-butoxide (1.0 M in THF, 79.3 mL) at −40° C. was added dropwise to a stirred solution of triphenyl(3,3,3-trifluoropropylidene)-λ⁵-phosphane (28.4 g, 79.3 mmol) in anh. THF (120 mL). The solution was then stirred at −20° C. for 1 h. Benzyl (S)-2-((tert-butoxycarbonyl)amino)-4-oxobutanoate (20.3 g, 66.1 mmol) was slowly added to the solution at −70° C. and the resulting mixture was stirred at −70° C. for 30 min. The reaction mixture was allowed to warm up to RT. The mixture was then slowly poured into an aq. sat. NH₄C₁ solution (300 mL) and extracted with EtOAc (200 mL). The organic layer was washed with brine (50 mL), dried over anh. Na₂SO₄, filtered, and concentrated in vacuum. The residue was purified by silica gel column chromatography, eluting with EtOAc 5-40% in PE to afford benzyl (S,E)-2-((tert-butoxycarbonyl)amino)-7,7,7-trifluorohept-4-enoate. ¹H NMR (400 MHz, chloroform-d): δ 7.40-7.36 (m, 5H), 5.64-5.52 (m, 2H), 5.22-5.11 (m, 2H), 4.48-4.45 (m, 1H), 2.82-2.77 (m, 2H), 2.75-2.63 (m, 1H), 2.54-2.51 (m, 1H), 1.44 (s, 9H).

Step 5: A solution of benzyl (S,E)-2-((tert-butoxycarbonyl)amino)-7,7,7-trifluorohept-4-enoate (12.6 g, 32.5 mmol) in THF (160 mL) was hydrogenated over Pd/C (1.30 g, 10 wt. %) o.n. at RT under 50 psi of dihydrogen. The reaction mixture was filtered through a Celite® pad. The solids were washed with THF (3×200 mL) and the combined filtrates were concentrated to give the crude product (S)-2-((tert-butoxycarbonyl)amino)-7,7,7-trifluoroheptanoic acid. MS ESI calculated for C₁₂H₂₀F₃NO₄ [M+Na]⁺ 322.12, found 322.2; MS ESI calculated for C₇H₁₂F₃NO₂ [M−Boc+H]⁺ 200.09, found 200.2. ¹H NMR (400 MHz, methanol-d₄): δ 4.11-4.05 (m, 1H), 2.16-2.11 (m, 2H), 1.67-1.60 (m, 2H), 1.59-1.49 (m, 4H), 1.44 (s, 9H).

Step 6: 4 M HCl in 1,4-dioxane (60 mL) was added to a stirred solution of (S)-2-((tert-butoxycarbonyl)amino)-7,7,7-trifluoroheptanoic acid (8.30 g, 27.7 mmol) in EtOAc (30 mL). The resulting solution was stirred at RT for 30 min. The solution was then concentrated under reduced pressure. The crude residue was triturated with MTBE (25 mL) at 15° C. for 20 min to give the crude product (S)-2-amino-7,7,7-trifluoroheptanoic acid. ¹H NMR (400 MHz, methanol-d₄): δ 4.00-3.97 (m, 1H), 2.21-2.17 (m, 2H), 1.96-1.94 (m, 2H), 1.65-1.55 (m, 4H).

Step 7: NaHCO₃(3.71 g, 44.2 mmol) and Fmoc-OSu (7.44 g, 22.1 mmol) at RT were added to a stirred solution of (S)-2-amino-7,7,7-trifluoroheptanoic acid (5.20 g, 22.1 mmol) in DI water (40 mL) and MeCN (40 mL). The resulting mixture was stirred at RT for 12 h. The pH was adjusted to 2 with aq. 1 N HCl solution. The mixture was extracted with EtOAc (3×100 mL). The combined organic layers were washed with brine (50 mL), dried over anh. Na₂SO₄, filtered, and concentrated in vacuum. The residue was purified by silica gel column chromatography, eluting with EtOAc 5-40% in PE, followed by chiral separation (column: Daicel Chiracel® OJ (250×50 mm, 10 μm, Daicel Chiral Technologies, West Chester, PA); mobile phase: [MeOH]; B %: 20%-20%, 8 min) to afford (S)-2-((((9H-fluoren-9-yl)methoxy)carbonyl)amino)-7,7,7-trifluoroheptanoic acid. MS ESI calculated for C₂₂H₂₂F3NO₄ [M+Na]⁺ 444.14, found 444.1; [M+H]⁺ 422.16, found 422.1. ¹H NMR (400 MHz, acetonitrile-d₃): δ 9.36 (br. s, 1H), 7.83 (d, J=7.2 Hz, 2H), 7.67-7.62 (m, 2H), 7.42 (d, J=7.6 Hz, 2H), 7.33 (d, J=7.6 Hz, 2H), 5.98-5.96 (m, 1H), 4.35-4.25 (m, 2H), 4.23-4.21 (m, 1H), 4.14-4.10 (m, 1H), 2.17-2.13 (m, 2H), 1.58-1.56 (m, 1H), 1.55-1.41 (m, 5H).

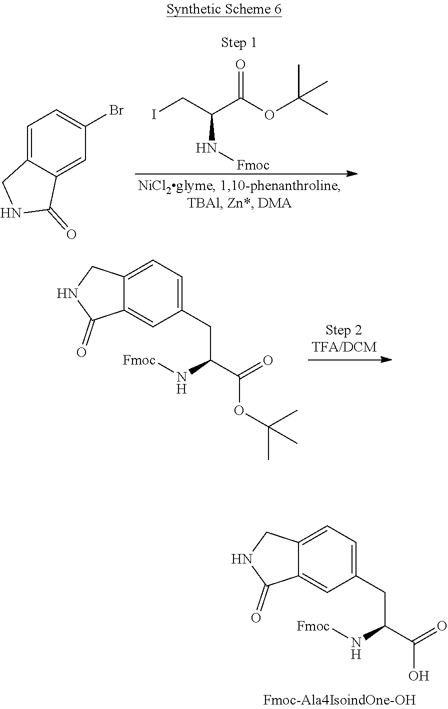

### (S)-2-((((9H-Fluoren-9-yl)methoxy)carbonyl)amino)-3-(3-oxoisoindolin-5-yl)propanoic acid (Fmoc-Ala4IsoindOne-OH)

Step 1: A mixture of nickel (II) chloride ethylene glycol dimethyl ether complex (0.53 g, 2.43 mmol) and 1,10-phenanthroline (0.44 g, 2.43 mmol) in anh. DMA (100 mL) was heated at 50° C. for 1 h. A mixture of TBAI (4.49 g, 12.16 mmol), 6-bromoisoindolin-1-one (2.58 g, 12.16 mmol), and tert-butyl (R)-2-((((9H-fluoren-9-yl)methoxy)carbonyl)amino)-3-iodopropanoate (6 g, 12.16 mmol) in anh. DMA (100 mL) was added at RT. Activated zinc (1.59 g, 24.32 mmol) was then added and stirred for 4 h at RT. The reaction was quenched with H₂O (400 mL) and extracted with EtOAc (2×500 mL). The combined organic layer was washed with brine (3×300 mL), dried over anh. Na₂SO₄ and filtered. The residue was purified by silica gel column chromatography, eluting with EtOAc 0-100% in PE to afford tert-butyl (S)-2-((((9H-fluoren-9-yl)methoxy)carbonyl)amino)-3-(3-oxoisoindolin-5-yl)propanoate. MS ESI calculated for C₃₀H₃₀N₂O₅Na [M+Na]⁺ 521.22, found 520.95.

Step 2: TFA (100 mL, 1298 mmol) at RT was added to a stirred solution of tert-butyl (S)-2-((((9H-fluoren-9-yl)methoxy)carbonyl)amino)-3-(3-oxoisoindolin-5-yl)propanoate (5.5 g, 11.03 mmol) in DCM (50 mL). The resulting mixture was stirred for 2.5 h at RT then concentrated under reduced pressure. The residue was recrystallized from Et₂O (100 mL) to give Fmoc-Ala4IsoindOne-OH. MS ESI calculated for C₂₆H₂₃N₂O₅ [M+H]⁺ 443.15, found 443.05. ¹H NMR (400 MHz, DMSO-d₆): δ 12.78 (s, 1H), 8.52 (s, 1H), 7.88-7.81 (m, 3H), 7.63-7.60 (m, 3H), 7.51-7.48 (m, 2H), 7.40-7.38 (m, 2H), 7.30-7.26 (m, 2H), 4.32 (s, 2H), 4.25-4.10 (m, 4H), 3.21-3.14 (m, 1H), 3.01-2.94 (m, 1H).

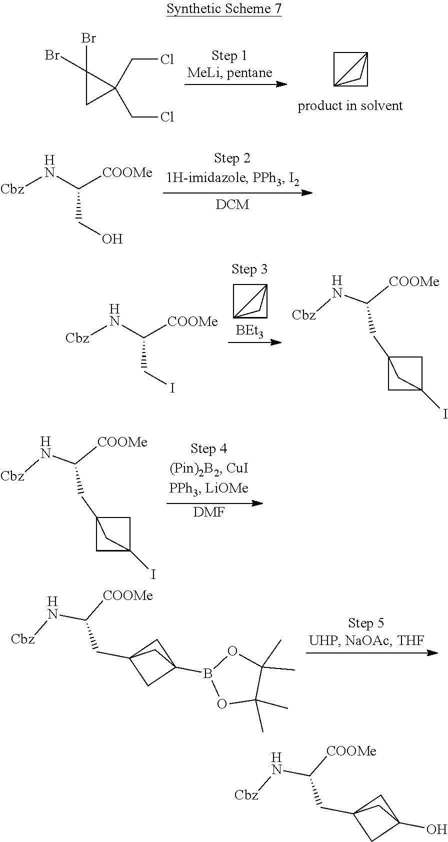

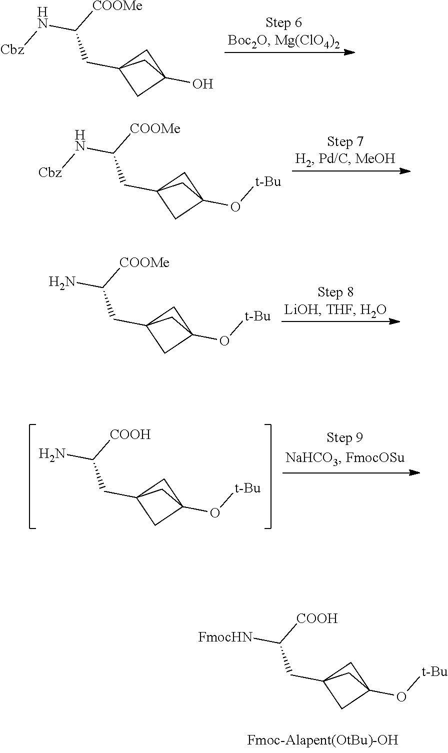

### (S)-2-((((9H-Fluoren-9-yl)methoxy)carbonyl)amino)-3-(3-(tert-butoxy)bicyclo[1.1.1]pentan-1-yl)propanoic acid (Fmoc-Alapent(OtBu)-OH)

Step 1: Into a 3-L 3-necked round-bottom flask was placed a solution of 1,1-dibromo-2,2-bis(chloromethyl)cyclopropane (200.0 g, 680.3 mmol) in pentane (2 L) under N₂. The mixture was cooled to −78° C. MeLi (2.0 M in Et₂O, 680.3 mL) was added dropwise over 2 h at −78° C. The reaction mixture was warmed to 0° C. over a period of 1 h. The reaction mixture was stirred for 1 h at 0° C. under N₂. NMR showed that the reaction was completed. The reaction mixture was purified by distillation under vacuum at 0° C. and the fraction was collected via condensation in a liquid nitrogen-ethanol bath. This resulted in tricyclo[1.1.1.01-3]pentane (2.75 wt. % in a mixed solution of pentane and ether). ¹H NMR (300 MHz, chloroform-d): δ 2.00 (d, J=1.3 Hz, 6H).

Step 2: Into a 2-L 3-neck round-bottom flask was placed a solution of PPh₃ (67.32 g, 256.6 mmol) in DCM (1000 mL) under N₂. Imidazole (17.47 g, 256.6 mmol) was added into the flask. The reaction mixture was cooled to 0° C. Diiodine (65.14 g, 256.60 mmol) was added into the flask in portions over 10 min at 0° C. The resulting mixture was stirred for 10 min at 0° C. Methyl ((benzyloxy)carbonyl)-L-serinate (50.00 g, 197.4 mmol) was added into the flask at 0° C. The resulting mixture was stirred for additional 4 h at 0° C. LC-MS monitoring showed that the reaction was completed. The resulting mixture was filtered. The filtered cake was washed with DCM (3×1000 mL). The filtrate was concentrated under reduced pressure. The residue was purified by silica gel column chromatography, eluting with EtOAc in PE (80:1-30:1; v/v) to afford methyl (2R)-2-[[(benzyloxy)carbonyl]amino]-3-iodopropanoate. MS ESI calculated for C₁₂H₁₄INO₄ [M+Na]⁺ 386, found 386. ¹H NMR (300 MHz, chloroform-d): δ 7.44-7.29 (m, 5H), 5.64 (d, J=7.5 Hz, 1H), 5.23-5.09 (m, 2H), 4.61 (dt, J=7.7, 3.8 Hz, 1H), 3.83 (s, 3H), 3.62 (t, J=3.4 Hz, 2H).

Step 3: Into a 3-L 3-neck round-bottom flask was placed a solution of tricyclo[1.1.1.0^{1,3}]pentane (880 g, 366.66 mmol, 2.75 wt. %) in a mixed solution of pentane and diethyl ether under N₂. The reaction mixture was cooled to 0° C. Methyl (R)-2-(((benzyloxy)carbonyl)amino)-3-iodopropanoate (55.00 g, 151.5 mmol) was added to the flask. Triethylborane (1.0 M in ether, 15.2 mL) was added dropwise over 5 min at 0° C. under N₂. The reaction mixture was naturally returned to RT and stirred for 30 min. LC-MS monitoring showed the reaction was completed. The resulting mixture was concentrated under reduced pressure. The residue was purified by silica gel column chromatography, eluting with EtOAc in PE (100:1-20:1; v/v) to afford methyl (2S)-2-(((benzyloxy)carbonyl)amino)-3-(3-iodobicyclo[1.1.1]pentan-1-yl)propanoate. MS ESI calculated for C₁₇H₂₀INO₄ [M+Na]⁺ 452, found 452. ¹H NMR (300 MHz, chloroform-d): δ 7.37 (d, J=4.5 Hz, 5H), 5.31 (d, J=8.3 Hz, 1H), 5.14 (t, J=2.9 Hz, 2H), 4.39 (td, J=7.8, 4.5 Hz, 1H), 3.75 (s, 3H), 2.30-2.09 (m, 7H), 1.92 (dd, J=14.7, 7.5 Hz, 1H).

Step 4: Into a 2-L 3-neck round-bottom flask was placed a solution of methyl (2S)-2-[[(benzyloxy)carbonyl]amino]-3-[3-iodobicyclo[1.1.1]pentan-1-yl]propanoate (40.00 g, 93.2 mmol) in DMF (600 mL). (Pin)₂B2 (35.50 g, 139.8 mmol), LiOMe (7.01 g, 184.5 mmol), and PPh₃ (3.67 g, 14.0 mmol) were added into flask in order at RT. The flask was evacuated and refilled with N₂ (reteated three times). CuI (1.77 g, 9.29 mmol) was added into the flask. The flask was evacuated and refilled with N₂ (repeated three times). The reaction mixture was stirred for 4 h at 35° C. LC-MS monitoring showed that the reaction was completed. The resulting mixture was diluted with water (900 mL). The resulting mixture was extracted with EtOAc (3×1000 mL). The organic layers were washed with brine (7×600 mL), dried over anh. Na₂SO₄, filtered, and concentrated under reduced pressure. This resulted in methyl (2S)-2-[[(benzyloxy) carbonyl]amino]-3-[3-(4,4,5,5-tetramethyl-1,3,2-dioxaborolan-2-yl)bicyclo[1.1.1]pentan-1-yl]propanoate. The crude was carried out to the next step without further purification.

Step 5: Into a 2-L 3-necked round-bottom flask was placed a solution of methyl (2S)-2-[[(benzyloxy)carbonyl]amino]-3-[3-(4,4,5,5-tetramethyl-1,3,2-dioxaborolan-2-yl)bicyclo[1.1.1]pentan-1-yl]propanoate (40.00 g, crude) in THF (600 mL, 15V). NaOAc (9.17 g, 111.8 mmol) and urea hydrogen peroxide (26.29 g, 279.5 mmol) were added into the flask at 0° C. The resulting mixture was stirred for 12 h at 30° C. under N₂. LC-MS monitoring showed that the reaction was completed. The resulting mixture was filtered. The filtered cake was washed with THF (3×100 mL). The filtrate was concentrated under reduced pressure. The residue was purified by silica gel column chromatography, eluting with EtOAc in PE (80:1-20:1; v/v) to afford methyl (2S)-2-[[(benzyloxy)carbonyl]amino]-3-[3-hydroxybicyclo[1.1.1]pentan-1-yl]propanoate. MS ESI calculated for C₁₇H₂₁NO₅ [M+Na]⁺ 342, found 342. ¹H NMR (400 MHz, chloroform-d): δ 7.43-7.30 (m, 5H), 5.24 (d, J=8.4 Hz, 1H), 5.14 (d, J=2.8 Hz, 2H), 4.41 (td, J=7.7, 4.5 Hz, 1H), 3.76 (s, 3H), 2.39 (s, 1H), 2.21 (dd, J=14.7, 4.6 Hz, 1H), 2.00 (dd, J=14.7, 7.3 Hz, 1H), 1.84 (s, 6H).

Step 6: Into a 250-mL 3-neck round-bottom flask was placed methyl (2S)-2-[[(benzyloxy)carbonyl]amino]-3-[hydroxybicyclo[1.1.1]pentan-1-yl]propanoate (16.00 g, 0.05 mmol). Boc₂O (54.67 g, 0.25 mmol) and magnesium perchlorate (1.17 g, 0.005 mmol) were added successively into the flask under N₂. The resulting mixture was stirred for 6 h at 50° C. LC-MS monitoring showed the reaction was completed. The residue was purified by silica gel column chromatography, eluting with EtOAc in PE (90:1-10:1; v/v) to afford methyl (2S)-2-[[(benzyloxy)carbonyl]amino]-3-[3-(tert-butoxy)bicyclo[1.1.1]pentan-1-yl]propanoate. MS ESI calculated for C₂₁H₂₉NO₅ [M+Na]⁺ 398, found 398. ¹H NMR (300 MHz, chloroform-d): δ 7.41-7.33 (m, 5H), 5.28-5.17 (m, 1H), 5.14 (d, J=4.0 Hz, 2H), 4.41 (q, J=7.9, 7.3 Hz, 1H), 3.76 (s, 3H), 2.28-2.13 (m, 1H), 1.92 (s, 7H), 1.25 (s, 9H).

Step 7: Into a 500-mL round-bottom flask was placed a solution of methyl (S)-2-(((benzyloxy)carbonyl)amino)-3-(3-(tert-butoxy)bicyclo[1.1.1]pentan-1-yl)propanoate (12.00 g) in MeOH (240.00 mL, 20V). The flask was evacuated and refilled with N₂ (5 times). Pd/C (anh. 2.40 g, 20 wt. %) was added into the flask. The flask was evacuated and backfilled with H₂ (5 times). The resulting mixture was stirred for 3 h at RT under an atmosphere of dihydrogen. LC-MS monitoring showed that the reaction was completed. The flask was evacuated and refilled with N₂ (5 times). The resulting mixture was filtered through a Celite® pad. The filtered cake was washed with MeOH (2×100 mL). The filtrate was concentrated under reduced pressure. This resulted in methyl (S)-2-amino-3-(3-(tert-butoxy)bicyclo[1.1.1]pentan-1-yl)propanoate. MS ESI calculated for C₁₃H₂₃NO₃ [M+H]⁺ 242, found 242. ¹H NMR (400 MHz, chloroform-d): δ 3.67 (s, 3H), 3.42 (dd, J=6.8, 5.5 Hz, 1H), 2.00 (dd, J=14.3, 5.5 Hz, 1H), 1.92-1.79 (m, 7H), 1.21 (s, 9H).

Step 8: Into a 500-mL 3-neck round-bottom flask was placed a solution of methyl (S)-2-amino-3-(3-(tert-butoxy)bicyclo[1.1.1]pentan-1-yl)propanoate (7.20 g, 19.2 mmol) in THF (144.00 mL, 20V). H₂O (72.00 mL, 10V) was added into the flask. LiOH (0.60 g, 25 mmol) was added into the flask at RT. The resulting mixture was stirred for 2 h at RT under N₂. LC-MS monitoring showed that the reaction was completed. The reaction mixture was engaged in the next step without further isolation.

Step 9: The reaction mixture was cooled to 0° C. NaHCO₃ (7.43 g, 0.08 mmol) and Fmoc-OSu (11.93 g, 0.03 mmol) were added into the flask. The resulting mixture was stirred for 2 h at 0° C. under N₂. LC-MS showed that the reaction was completed. The mixture was adjusted pH ˜3-4 with 1 N HCl. The resulting mixture was extracted with EtOAc (3×150 mL). The organic phase was washed with water (3×100 mL) to remove residual HCl. The organic layer was washed with brine (200 mL) and dried over anh. Na₂SO₄. After filtration, the filtrate was concentrated under reduced pressure. The residue was purified by Rp flash chromatography using a C18 silica gel column; mobile phase: MeCN/water, 15-50% gradient of MeCN over 40 min; detector: UV 210 nm. The fractions containing the product were concentrated under vacuum to afford (2S)-3-[3-(tert-butoxy)bicyclo[1.1.1]pentan-1-yl]-2-[[(9H-fluoren-9-ylmethoxy)carbonyl]amino]propanoic acid. MS ESI calculated for C₂₇H₃₁NO₅ [M+Na]⁺ 472, found 472. ¹H NMR (400 MHz, DMSO-d₆): δ 12.57 (s, 1H), 7.90 (d, J=7.5 Hz, 2H), 7.73 (t, J=6.9 Hz, 2H), 7.62 (d, J=8.5 Hz, 1H), 7.42 (td, J=7.5, 1.2 Hz, 2H), 7.32 (tt, J=7.5, 1.2 Hz, 2H), 4.38-4.20 (m, 3H), 4.03 (q, J=7.1 Hz, 1H), 3.93 (ddd, J=12.7, 6.4, 2.7 Hz, 1H), 2.00 (dd, J=14.4, 3.8 Hz, 1H), 1.92-1.86 (m, 1H), 1.85-1.75 (m, 6H), 1.16 (s, 8H).

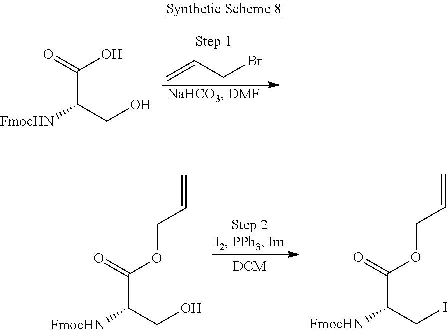

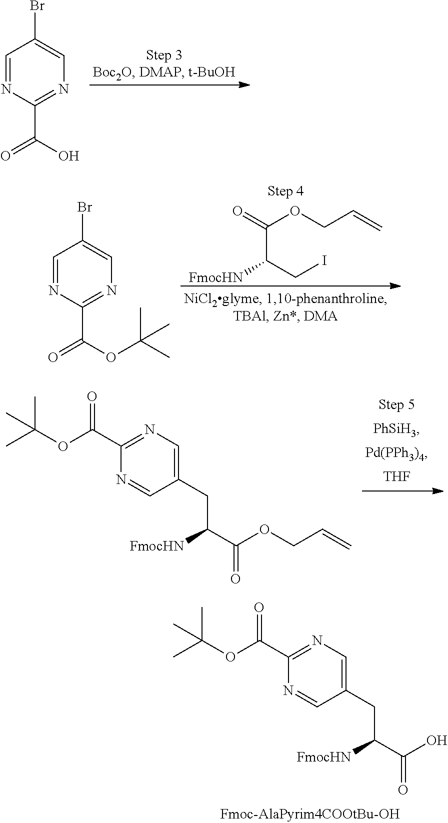

### (S)-2-((((9H-Fluoren-9-yl)methoxy)carbonyl)amino)-3-(2-(tert-butoxycarbonyl)pyrimidin-5-yl)propanoic acid (Fmoc-AlaPyrim4COOtBu-OH)

Step 1: To a solution of (((9H-fluoren-9-yl)methoxy)carbonyl)-L-serine (10.0 g, 30.5 mmol) in DMF (150 mL) were added NaHCO₃ (12.8 g, 153.0 mmol) and 3-bromoprop-1-ene (11.1 g, 92.0 mmol) at 0° C. under Ar atmosphere. The resulting mixture was stirred for 12 h at RT. The mixture was diluted with water (500 mL) and extracted with EtOAc (1000 mL). The combined organic layer was washed with brine (3×500 mL), dried over anh. Na₂SO₄ and filtered. The filtrate was concentrated under reduced pressure. The residue was purified by silica gel column chromatography, eluting with EtOAc 0-50% in PE to give allyl (((9H-fluoren-9-yl)methoxy)carbonyl)-L-serinate. MS ESI calculated for C₂₁H₂₁NO₅Na [M+Na]⁺ 390.14, found 390.20. ¹H NMR (300 MHz, chloroform-d): δ 7.80-7.21 (m, 8H), 5.99-5.67 (m, 2H), 5.40-5.19 (m, 2H), 4.69-4.67 (m, 2H), 4.43-4.41 (m, 3H), 4.22-4.20 (m, 1H), 4.04-3.79 (m, 2H), 2.17 (s, 1H).

Step 2: Imidazole (3.6 g, 52.3 mmol), PPh₃ (11.7 g, 44.4 mmol), and iodine (9.9 g, 39.2 mmol) were added sequentially at RT to a mixture of allyl (((9H-fluoren-9-yl)methoxy)carbonyl)-L-serinate (9.6 g, 26.1 mmol) in DCM (960 mL). The reaction was stirred at RT for 4 h. The resulting solution was quenched with sat. Na₂S₂O₃ (300 mL) and extracted with DCM (2×500 mL). The organic layers were combined, washed with brine (2×400 mL), dried over anh. Na₂SO₄ and filtered. The filtrate was concentrated under reduced pressure. The residue was purified by silica gel chromatography, eluting with EtOAc 0-25% in PE to afford allyl (R)-2-((((9H-fluoren-9-yl)methoxy)carbonyl)amino)-3-iodopropanoate. MS ESI calculated for C₂₁H₂₀INO₄Na [M+Na]⁺ 500.04, found 500.15. ¹H NMR (400 MHz, chloroform-d): δ 7.78-7.76 (m, 2H), 7.63-7.61 (m, 2H), 7.41-7.39 (m, 2H), 7.33-7.31 (m, 2H), 5.94-5.92 (m, 1H), 5.68-5.66 (m, 1H), 5.49-5.22 (m, 2H), 4.74-4.73 (m, 2H), 4.62-4.60 (m, 1H), 4.41-4.40 (m, 2H), 4.25-4.24 (m, 1H), 3.72-3.52 (m, 2H).

Step 3: DMAP (0.3 g, 2.5 mmol) and Boc₂O (6.5 g, 29.6 mmol) under Ar atmospher and at RT were added to a mixture of 5-bromopyrimidine-2-carboxylic acid (5.0 g, 24.6 mmol) in t-BuOH (75 mL). The reaction was stirred o.n. at 50° C. The reaction was cooled to RT and concentrated in vacuum. The residue was purified by silica gel column chromatography, eluting with EtOAc 0-20% in PE to give tert-butyl 5-bromopyrimidine-2-carboxylate. MS ESI calculated for C₉H₁₁BrN₂O₂[M−tBu+H]⁺ 203.00/205.00, found 202.95/204.95.

Step 4: A mixture of nickel (II) chloride ethylene glycol dimethyl ether complex (63.6 mg, 0.29 mmol) and pyridine-2-carboximidamide hydrochloride (91 mg, 0.58 mmol) in anh. DMA (20 mL) was heated at 50° C. for 1 h. A solution of allyl (R)-2-((((9H-fluoren-9-yl)methoxy)carbonyl)amino)-3-iodopropanoate (1.38 g, 2.89 mmol), tert-butyl 5-bromopyrimidine-2-carboxylate (750 mg, 2.89 mmol), and TBAI (2.14 g, 5.79 mmol) in anh. DMA (25 mL) was added at RT. Activated zinc (378 mg, 5.79 mmol) was then added and the mixture was stirred at RT for 2 h. The reaction was quenched with H₂O (200 mL) and extracted with EtOAc (2×400 mL). The combined organic layers were washed with brine (3×200 mL), dried over anh. Na₂SO₄ and filtered. The filtrate was concentrated under reduced pressure. The residue was purified by silica gel column chromatography, eluting with EtOAc 0-45% in PE to afford tert-butyl (S)-5-(2-((((9H-fluoren-9-yl)methoxy)carbonyl)amino)-3-(allyloxy)-3-oxopropyl)pyrimidine-2-carboxylate. MS ESI calculated for C₃₀H₃₂N₃O₆ [M+H]⁺ 530.22, found 530.25.

Step 5: Pd(PPh₃)₄(0.16 g, 0.14 mmol) and phenylsilane (0.59 g, 5.44 mmol) at RT were added to a stirred solution of tert-butyl (S)-5-(2-((((9H-fluoren-9-yl)methoxy)carbonyl)amino)-3-(allyloxy)-3-oxopropyl)pyrimidine-2-carboxylate (1.44 g, 2.72 mmol) in THF (25 mL). The resulting mixture was stirred at RT for 1 h. The solvent was concentrated under reduced pressure. The residue was purified by Rp-flash column chromatography with the following conditions: Column: Flash C18 (330 g); Mobile Phase A: water (0.1% TFA), Mobile Phase B: MeCN (0.1% TFA); Gradient Elution: 2-50%; Detector: UV 215 nm. The fractions containing the product were concentrated under reduced pressure to afford (S)-2-((((9H-fluoren-9-yl)methoxy)carbonyl) amino)-3-(2-(tert-butoxycarbonyl)pyrimidin-5-yl)propanoic acid. MS ESI calculated for C₂₇H₂₈N₃O₆ [M+H]⁺ 490.19, found 490.15. ¹H NMR (300 MHz, DMSO-d₆): δ 8.86 (s, 2H), 7.86-7.85 (m, 3H), 7.69-7.52 (m, 2H), 7.46-7.24 (m, 4H), 4.33-4.32 (m, 1H), 4.20-4.18 (m, 3H), 3.23-3.21 (m, 1H), 2.98-2.97 (m, 1H), 1.55 (s, 9H).

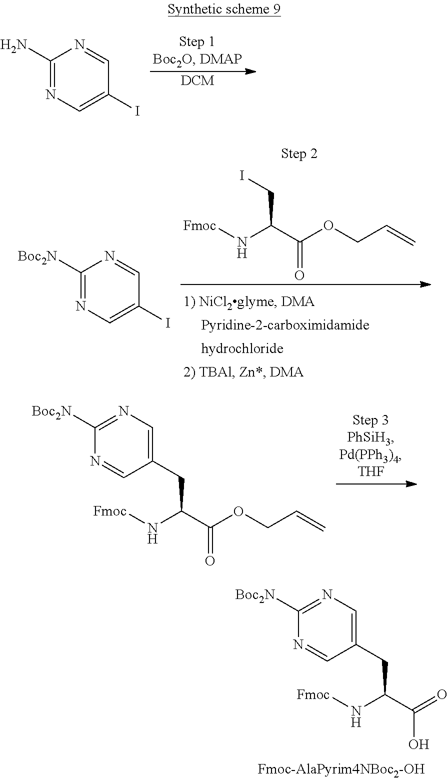

### (S)-2-((((9H-Fluoren-9-yl)methoxy)carbonyl)amino)-3-(2-(bis(tert-butoxycarbonyl)amino)pyrimidin-5-yl)propanoic acid (Fmoc-AlaPyrim4NBoc₂-OH)

Step 1: DMAP (0.17 g, 1.36 mmol) and Boc₂O (7.5 mL, 32.6 mmol) were added to a mixture of 5-iodopyrimidin-2-amine (3.00 g, 13.6 mmol) in DCM (45 mL) under an Ar atmosphere at RT. The mixture was stirred o.n. at RT. The mixture was concentrated under reduced pressure and the residue was purified by silica gel column chromatography, eluting with EtOAc 0-30% in PE to afford tert-butyl (tert-butoxycarbonyl)(5-iodopyrimidin-2-yl)carbamate. MS ESI calculated for C₁₄H₂₁IN₃O₄[M+H]⁺ 422.05, found 422.00. ¹H NMR (300 MHz, chloroform-d): δ 8.91 (s, 2H), 1.47 (s, 18H).

Step 2: A mixture of nickel (II) chloride ethylene glycol dimethyl ether complex (0.26 g, 1.19 mmol) and pyridine-2-carboximidamide hydrochloride (0.37 g, 2.37 mmol) in anh. DMA (100 mL) was heated at 50° C. for 1 h. A combination of allyl (R)-2-((((9H-fluoren-9-yl)methoxy) carbonyl)amino)-3-iodopropanoate (5.67 g, 11.9 mmol), tert-butyl (tert-butoxycarbonyl)(5-iodopyrimidin-2-yl)carbamate (5.00 g, 11.9 mmol), and TBAI (8.77 g, 23.7 mmol) in anh. DMA (120 mL) was added at RT, followed by activated zinc (1.55 g, 23.7 mmol). The resulting mixture was stirred at RT for 4 h. The mixture was quenched with H₂O (600 mL) and extracted with EtOAc (2×400 mL). The combined organic layers were washed with brine (2×300 mL), dried over anh. Na₂SO₄, filtered, and concentrated under reduced pressure. The residue was purified by silica gel column chromatography, eluting with EtOAc 0-60% in PE to afford allyl (S)-2-((((9H-fluoren-9-yl)methoxy)carbonyl)amino)-3-(2-(bis(tert-butoxycarbonyl)amino) pyrimidin-5-yl)propanoate. MS ESI calculated for C₃₅H₄₁N₄O₈[M+H]⁺ 645.28, found 645.10. ¹H NMR (400 MHz, DMSO-d₆): δ 8.78 (s, 2H), 8.02 (d, J=8.6 Hz, 1H), 7.88 (d, J=7.5 Hz, 2H), 7.65-7.63 (m, 2H), 7.44-7.37 (m, 2H), 7.32-7.30 (m, 2H), 5.90-5.88 (m, 1H), 5.34-5.33 (m, 1H), 5.23-5.22 (m, 1H), 4.62 (d, J=5.3 Hz, 2H), 4.44-4.43 (m, 1H), 4.32-4.21 (m, 1H), 4.21-4.11 (m, 2H), 3.20-3.19 (m, 1H), 2.96-2.95 (m, 1H), 1.35 (s, 18H).

Step 3: Pd(PPh₃)₄(0.33 g, 0.29 mmol) and phenylsilane (1.24 g, 11.5 mmol) were added to a mixture of allyl (S)-2-((((9H-fluoren-9-yl)methoxy)carbonyl)amino)-3-(2-(bis(tert-butoxycarbonyl)amino)pyrimidin-5-yl)propanoate (3.7 g, 5.7 mmol) in anh. THF (70 mL) at RT. The mixture was stirred at RT for 3 h. The reaction mixture was concentrated under reduced pressure. The residue was purified by Rp-flash column chromatography with the following conditions: Column: Flash C18 (330 g); Mobile Phase A: water (0.1% TFA), Mobile Phase B: MeCN (0.1% TFA); Gradient Elution: 25-55%; Detector: UV 215 nm. The fractions containing the product were concentrated under reduced pressure to afford (S)-2-((((9H-Fluoren-9-yl) methoxy)carbonyl) amino)-3-(2-(bis(tert-butoxycarbonyl)amino)pyrimidin-5-yl)propanoic acid. MS ESI calculated for C₃₂H₃₇N₄O₈ [M+H]⁺ 605.25, found 605.10. ¹H NMR (400 MHz, DMSO-d₆): δ 12.81 (s, 1H), 8.76 (s, 2H), 7.89-7.87 (m, 3H), 7.67-7.66 (m, 2H), 7.44-7.38 (m, 2H), 7.37-7.34 (m, 2H), 4.23-4.13 (m, 4H), 3.18-3.08 (m, 2H), 1.34 (s, 18H).

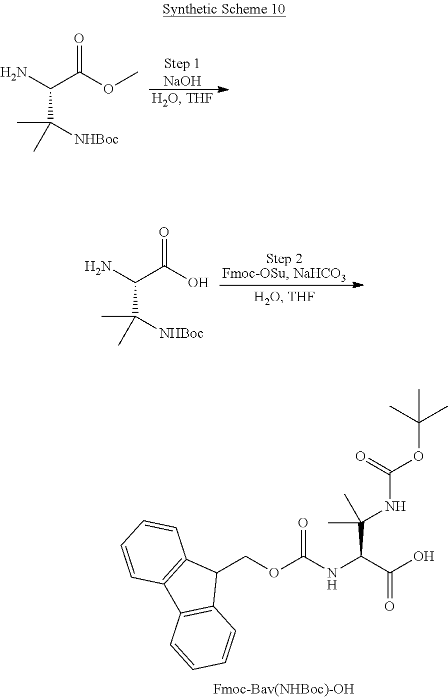

### (S)-2-((((9H-Fluoren-9-yl)methoxy)carbonyl)amino)-3-((tert-butoxycarbonyl)amino)-3-methylbutanoic acid (Fmoc-Bav(NHBoc)-OH)

Step 1: Sodium hydroxide (1.0 g, 25 mmol) was added to a stirred solution of methyl (S)-2-amino-3-((tert-butoxycarbonyl)amino)-3-methylbutanoate (1.0 g, 4.1 mmol) in DI water (40 mL) and THF (40 mL). The solution was stirred at RT for 2 h. The pH was adjusted to pH 7 by addition of aq. 1 N HCl solution.

Step 2: NaHCO₃(1.0 g, 1.9 mmol) and Fmoc-OSu (1.5 g, 4.5 mmol) were added to the solution of Step 1. The reaction mixture was stirred at RT for 4 h. The pH was adjusted to 2 with aq. 1 N HCl solution and extracted with EtOAc (2×80 mL). The combined organic layers were washed with brine (50 mL), dried over anh. Na₂SO₄ and filtered. The filtrate was concentrated under reduced pressure and the residue was purified by silica gel column chromatography, eluting with 0-10% MeOH in DCM to afford (S)-2-((((9H-fluoren-9-yl)methoxy)carbonyl) amino)-3-((tert-butoxycarbonyl) amino)-3-methylbutanoic acid. MS ESI calculated for C₂₅H₃₀N₂O₆[M+Na]⁺ 477.20, found 477.2; [M-Boc+H]⁺ 354.16, found 355.2. ¹H NMR (600 MHz, DMSO-d₆): δ 12.59 (s, 1H), 7.90 (d, J=7.6 Hz, 2H), 7.73 (m, 2H), 7.61 (d, J=8.5 Hz, 1H), 7.42 (t, J=7.5 Hz, 2H), 7.32 (t, J=7.4 Hz, 2H), 6.56 (s, 1H), 4.57 (d, J=8.4 Hz, 1H), 4.30-4.20 (m, 3H), 1.36 (s, 9H), 1.27 (s, 6H). ¹³C NMR (151 MHz, DMSO-d₆): δ 172.2, 156.1, 154.6, 143.8, 140.7, 127.7, 127.1, 125.4, 120.2, 107.0, 66.6, 65.8, 59.7, 46.6, 28.3, 24.8, 24.2.

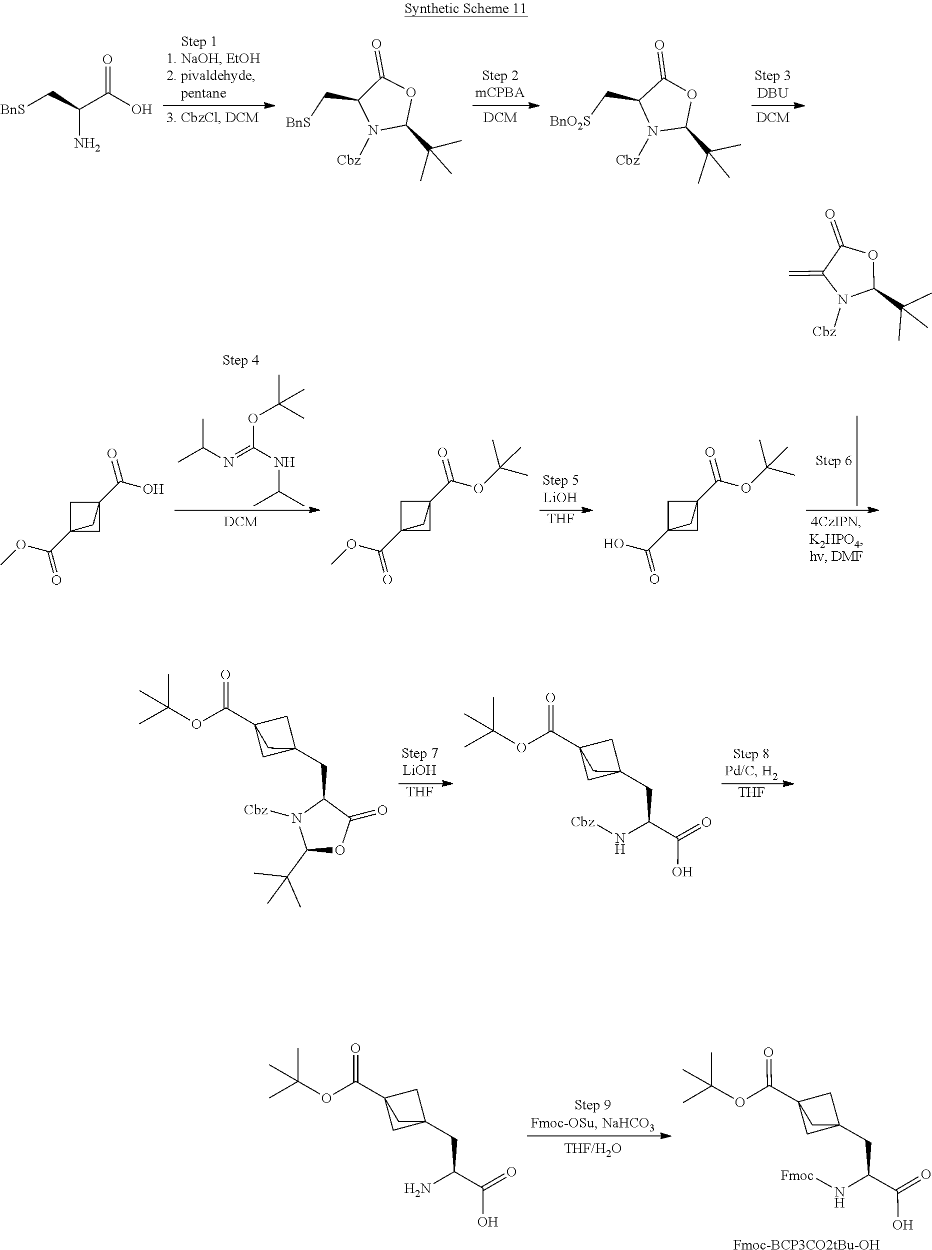

### (S)-2-((((9H-Fluoren-9-yl)methoxy)carbonyl)amino)-3-(3-(tert-butoxycarbonyl)bicyclo[1.1.1]pentan-1-yl)propanoic acid (Fmoc-BCP3COOtBu-OH)

Step 1: An aq. 1 N NaOH (1.0 M, 47.3 mL, 47.3 mmol) was added to a solution of S-benzyl-L-cysteine (10.0 g, 47 mmol) in EtOH (150 mL) at RT. The mixture was stirred at RT for 1 h. The mixture was concentrated under reduced pressure. The residue was suspended in pentane (300 mL). Pivaldehyde (20.4 g, 237 mmol) and activated 3 Å molecular sieves were added at RT. The mixture was heated to reflux (−40° C.) and stirred for 6 h. The mixture was cooled to RT and filtered. The filtrate was concentrated under reduced pressure. The residue was azeotropically dried with toluene (3×200 mL). The residue was dissolved in anh. DCM (300 mL) and CbzCl (13.5 mL, 95.0 mmol) was added at 0° C. The mixture was stirred o.n. at RT. The mixture was concentrated under reduced pressure and the residue was purified by silica gel column chromatography, eluting with EtOAc 0-30% in PE to afford benzyl (2S,4R)-4-((benzylthio)methyl)-2-(tert-butyl)-5-oxooxazolidine-3-carboxylate. MS ESI calculated for C₂₃H₂₈NO₄S [M+H]⁺ 414.17, found 414.05. ¹H NMR (300 MHz, chloroform-d): δ 7.38-7.30 (m, 5H), 7.25-7.03 (m, 5H), 5.53 (s, 1H), 5.27-5.12 (m, 2H), 4.55-4.50 (m, 1H), 3.86-3.66 (m, 2H), 2.98-2.72 (m, 2H), 0.91 (s, 9H).

Step 2: mCPBA (10.9 g, 53.8 mmol) was added to a mixture of benzyl (2S,4R)-4-((benzylthio)methyl)-2-(tert-butyl)-5-oxooxazolidine-3-carboxylate (8.9 g, 22 mmol) in DCM (180 mL) at 0° C. The mixture was stirred at RT for 3 h. The mixture was quenched with 1N aq. NaOH (150 mL) and extracted with DCM (3×200 mL). The combined organic layers were washed with brine (3×200 mL), dried over anh. Na₂SO₄ and filtered. The filtrate was concentrated under reduced pressure and the residue was purified by silica gel column chromatography, eluting with EtOAc 0-35% in PE to afford benzyl (2S,4R)-4-((benzylsulfonyl) methyl)-2-(tert-butyl)-5-oxooxazolidine-3-carboxylate. MS ESI calculated for C₂₃H₂₈NO₆S [M+H]⁺ 446.16, found 446.05. ¹H NMR (400 MHz, chloroform-d): δ 7.44-7.32 (m, 10H), 5.62 (s, 1H), 5.28-5.17 (m, 2H), 5.10-5.07 (m, 1H), 4.70-4.65 (m, 1H), 4.44-4.40 (m, 1H), 3.47-3.41 (m, 1H), 3.17-3.12 (m, 1H), 0.89 (s, 9H).

Step 3: DBU (2.89 g, 19.0 mmol) was added to a solution of benzyl (2S,4R)-4-((benzylsulfonyl)methyl)-2-(tert-butyl)-5-oxooxazolidine-3-carboxylate (7.70 g, 17.3 mmol) in DCM (155 mL) at 0° C. The mixture was stirred at 0° C. for 1 h. The mixture was concentrated under reduced pressure and the residue was purified by silica gel column chromatography, eluting with EtOAc 0-30% in PE to afford benzyl (S)-2-(tert-butyl)-4-methylene-5-oxooxazolidine-3-carboxylate. MS ESI calculated for C₁₆H₂₀NO₄ [M+H]⁺ 290.13, found 290.10. ¹H NMR (300 MHz, chloroform-d): δ 7.38-7.36 (m, 5H), 5.75-5.63 (m, 3H), 5.29-5.21 (m, 2H), 0.93 (s, 9H).

Step 4: tert-Butyl (Z)—N,N′-diisopropylcarbamimidate (31.0 mL, 147 mmol) was added to a solution of 3-(methoxycarbonyl)bicyclo[1.1.1]pentane-1-carboxylic acid (5.0 g, 29.4 mmol) in DCM (75 mL) at RT. The reaction mixture was stirred at 40° C. for 2 h. The mixture was cooled to RT. The mixture was filtered and the filtrate was concentrated under reduced pressure to afford 1-(tert-butyl) 3-methyl bicyclo[1.1.1]pentane-1,3-dicarboxylate, which was used in the next step without further purification. ¹H NMR (300 MHz, chloroform-d): δ 3.69 (s, 3H), 2.32 (s, 6H), 1.45 (s, 9H).

Step 5: An aq. solution of LiOH (1.0 M, 46.8 mL, 47 mmol) was added to a mixture of 1-(tert-butyl) 3-methyl bicyclo[1.1.1]pentane-1,3-dicarboxylate (5.3 g, 23 mmol) in THF (53 mL) at 0° C. The mixture was stirred at RT for 3 h. The mixture was cooled to 0° C. and the pH was adjusted to ˜3-4 with aq. 1 N HCl (46.8 mL). The mixture was extracted with EtOAc (3×200 mL). The organic layers were combined, washed with brine (3×200 mL), dried over anh. Na₂SO₄ and filtered. The filtrate was concentrated under reduced pressure to afford 3-(tert-butoxycarbonyl)bicyclo[1.1.1]pentane-1-carboxylic acid, which was used without purification in the next step. MS ESI calculated for C₁H₁₅O₄[M−H]⁻ 211.10, found 211.10. ¹H NMR (300 MHz, methanol-d₄): δ 2.21 (s, 6H), 1.44 (s, 9H).

Step 6: 3-(tert-Butoxycarbonyl)bicyclo[1.1.1]pentane-1-carboxylic acid (1.10 g, 5.18 mmol), 1,2,3,5-tetrakis(carbazol-9-yl)-4,6-dicyanobenzene (0.06 g, 0.07 mmol), and K₂HPO₄ (1.51 g, 8.64 mmol) were added to a solution of benzyl (S)-2-(tert-butyl)-4-methylene-5-oxooxazolidine-3-carboxylate (1.00 g, 3.46 mmol) in DMF (33 mL). The mixture was irradiated with a 34 W blue LED lamp and stirred o.n. at RT under Ar. The mixture was diluted with water (150 mL) and extracted with EtOAc (3×200 mL). The combined organic layers were washed with brine (3×200 mL), dried over anh. Na₂SO₄ and filtered. The filtrate was concentrated under reduced pressure and the residue was purified by silica gel column chromatography, eluting with EtOAc 0-25% in PE to afford benzyl (2S,4S)-4-((3-(tert-butoxycarbonyl)bicyclo[1.1.1]pentan-1-yl)methyl)-2-(tert-butyl)-5-oxooxazolidine-3-carboxylate. MS ESI calculated for C₂₆H₃₉N₂O₆ [M+NH₄]⁺ 475.25, found 475.30. ¹H NMR (400 MHz, chloroform-d): δ 7.44-7.32 (m, 5H), 5.53 (s, 1H), 5.22-5.11 (m, 2H), 4.28-4.21 (m, 1H), 2.14-2.08 (m, 1H), 2.01-1.90 (m, 7H), 1.43 (s, 9H), 0.94 (s, 9H).

Step 7: An aq. solution of LiOH (1.0 M, 10.4 mL, 10 mmol) was added to a mixture of benzyl (2S,4S)-4-((3-(tert-butoxycarbonyl)bicyclo[1.1.1]pentan-1-yl)methyl)-2-(tert-butyl)-5-oxooxazolidine-3-carboxylate (1.9 g, 4.2 mmol) in THF (40 mL) at 0° C. The mixture was stirred at RT for 4 h. The mixture was cooled to 0° C. and the pH value was adjusted to ˜3-4 with aq. 1 M HCl (10 mL). The mixture was diluted with water (60 mL) and extracted with EtOAc (3×100 mL). The combined organic layers were washed with brine (3×100 mL), dried over Na₂SO₄, and filtered. The filtrate was concentrated under reduced pressure to afford (S)-2-(((benzyloxy) carbonyl)amino)-3-(3-(tert-butoxycarbonyl)bicyclo[1.1.1]pentan-1-yl)propanoic acid, which was used in the next step without further purification. MS ESI calculated for C₂₁H₃₁N₂O₆ [M+NH₄]⁺ 407.22, found 407.10. ¹H NMR (400 MHz, chloroform-d): δ 7.39-7.29 (m, 5H), 5.26-5.23 (m, 1H), 5.12 (s, 2H), 4.44-4.38 (m, 1H), 2.17-2.09 (m, 1H), 1.98-1.85 (m, 7H), 1.42 (s, 9H).

Step 8: (S)-2-(((Benzyloxy)carbonyl)amino)-3-(3-(tert-butoxycarbonyl)bicyclo[1.1.1]pentan-1-yl)propanoic acid (1.9 g, 4.9 mmol) was dissolved in THF (40 mL). The flask was evacuated and refilled with N₂ (5 times). Pd/C (1.04 g, 0.98 mmol, dry, 10 wt. %) was added into the flask. The flask was evacuated and backfilled with H₂ (5 times). The resulting mixture was stirred for 2 h at RT under an atmosphere of dihydrogen. The flask was evacuated and refilled with N₂ (5 times). The resulting mixture was filtered through a Celite® pad. The filtered cake was washed with THF (2×25 mL). The filtrate was concentrated under reduced pressure. The reaction mixture of ((S)-2-amino-3-(3-(tert-butoxycarbonyl)bicyclo[1.1.1]pentan-1-yl)propanoic acid (1.2 g, 4.70 mmol, 96% yield)) was used in the next step without further manipulation. MS ESI calculated for C₁₃H₂₂NO₄ [M+H]⁺ 256.15, found 256.15.

Step 9: The reaction mixture of (S)-2-amino-3-(3-(tert-butoxycarbonyl)bicyclo[1.1.1]pentan-1-yl)propanoic acid (1.2 g, 4.7 mmol) in THF (40 mL) from Step 8 was diluted with water (40 mL). NaHCO₃ (1.97 g, 23.5 mmol) and Fmoc-OSu (1.59 g, 4.70 mmol) were added at 0° C. The mixture was stirred o.n. at RT. The mixture was filtered, and the filter cake was washed with THF (2×100 mL). The filtrate was partially concentrated under reduced pressure. The pH of the solution was adjusted to ˜3-4 with aq. 1 N HCl (23.5 mL). The mixture was extracted with EtOAc (3×150 mL). The combined organic layers were washed with brine (3×150 mL), dried over anh. Na₂SO₄ and filtered. The filtrate was concentrated under reduced pressure and the residue was purified by silica gel column chromatography eluting with MeOH 0-15% in DCM to afford (S)-2-((((9H-fluoren-9-yl)methoxy)carbonyl)amino)-3-(3-(tert-butoxycarbonyl)bicyclo[1.1.1]pentan-1-yl)propanoic acid. MS ESI calculated for C₂₈H₃₁NO₆Na [M+Na]⁺ 500.22, found 500.35. ¹H NMR (400 MHz, methanol-d₄): δ 7.79 (d, J=7.6 Hz, 2H), 7.69-7.65 (m, 2H), 7.39-7.35 (m, 2H), 7.32-7.28 (m, 2H), 4.46-4.44 (m, 1H), 4.38-4.35 (m, 1H), 4.24-4.20 (m, 1H), 4.11-4.08 (m, 1H), 2.05-2.01 (m, 1H), 1.89-1.74 (m, 7H), 1.44 (s, 9H).

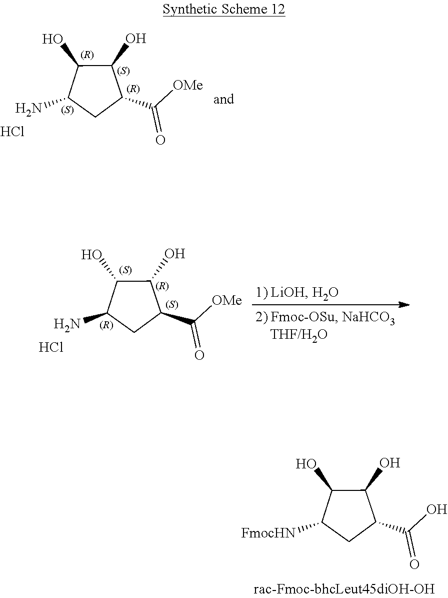

### (1R,2S,3R,4S) and (1S,2R,3S,4R)-4-((((9H-Fluoren-9-yl)methoxy)carbonyl)amino)-2,3-dihydroxycydopentane-1-carboxylic acid (Fmoc-bhcLeut45diOH-OH)

Lithium hydroxide (200 mg, 8.35 mmol) was added to a racemic mixture of (1R,2S,3R,4S)- and (1S,2R,3S,4R)-4-amino-2,3-dihydroxycyclopentane-1-carboxylate hydrochloride (500 mg, 2.36 mmol) in water (15 mL). The mixture was stirred at RT for 2 h. The pH was adjusted to ˜4 by addition of aq. 1 N HCl, and then THF (15 mL) was added. Fmoc-OSu (0.95 g, 2.8 mmol) and sat. aq. NaHCO₃ (10 mL) were added to the mixture. The mixture was stirred at RT for 18 h. The resulting mixture was concentrated under reduced pressure. The mixture was adjusted to pH ˜3 with aq. 1 N HCl and then extracted with EtOAc (2×80 mL). The combined organic layers were dried over anh. Na₂SO₄, filtered, and concentrated under reduced pressure. The residue was purified by silica gel chromatography, eluting with MeOH in DCM to afford (1R,2S,3R,4S)- and (1S,2R,3S,4R)-4-((((9H-fluoren-9-yl)methoxy)carbonyl)amino)-2,3-dihydroxycyclopentane-1-carboxylic acid as a racemic mixture. MS ESI calculated for C₂₁H₂₂NO₆ [M+H]⁺ 384.1, found 384.1. ¹H NMR (600 MHz, DMSO-d₆): δ 12.19 (s, 1H), 7.90 (d, J=7.5 Hz, 2H), 7.72 (d, J=7.4 Hz, 2H), 7.44-7.39 (m, 3H), 7.36-7.32 (m, 2H), 4.88-4.64 (m, 2H), 4.30-4.27 (m, 2H), 4.22 (t, J=6.9 Hz, 1H), 4.00 (t, J=5.2 Hz, 1H), 3.76-3.69 (m, 1H), 3.63 (t, J=5.3 Hz, 1H), 2.61-2.56 (m, 1H), 2.21-2.14 (m, 1H), 1.52-1.45 (m, 1H).

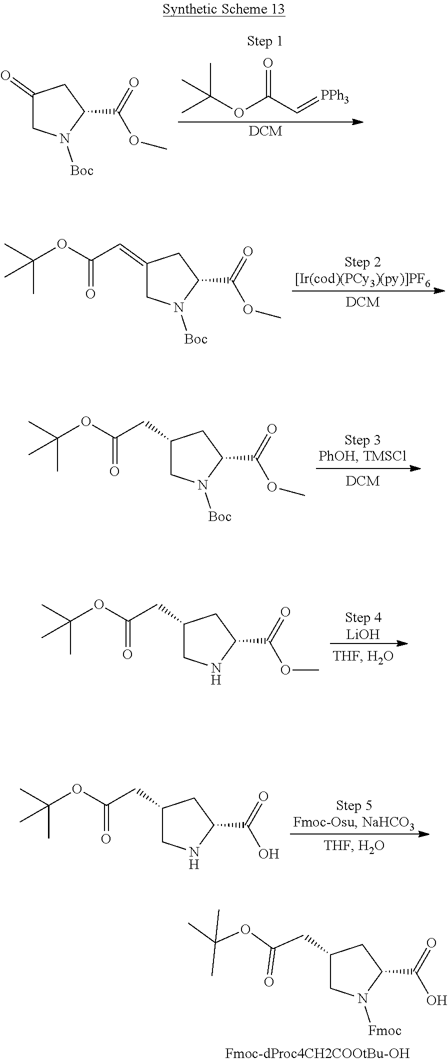

### (2R,4S)-1-(((9H-Fluoren-9-yl)methoxy)carbonyl)-4-(2-(tert-butoxy)-2-oxoethyl)pyrrolidine-2-carboxylic acid (Fmoc-dProc4CH2COOtBu-OH)

Step 1: A mixture of 1-(tert-butyl) 2-methyl (R)-4-oxopyrrolidine-1,2-dicarboxylate (4.00 g, 16.4 mmol) and tert-butyl 2-(triphenyl-15-phosphaneylidene)acetate (15.47 g, 41.1 mmol) in DCM (80 mL) was stirred and heated o.n. at 40° C. The mixture was concentrated under reduced pressure. The residue was purified by a silica gel column chromatography eluting with EtOAc 0-60% in PE to afford 1-(tert-butyl) 2-methyl (R,Z)-4-(2-(tert-butoxy)-2-oxoethylidene)pyrrolidine-1,2-dicarboxylate. MS ESI calculated for C₁₇H₂₇NO₆Na [M+Na]⁺ 364.18, found 364.00.

Step 2: 1-(tert-Butyl) 2-methyl (R,Z)-4-(2-(tert-butoxy)-2-oxoethylidene)pyrrolidine-1,2-dicarboxylate (5.00 g, 14.7 mmol) and (1,5-cyclooctadiene)(pyridine)(tricyclohexylphosphine)-iridium(I) hexafluorophosphate (2.36 g, 2.93 mmol) were dissolved in DCM (100 mL). The flask was evacuated and backfilled with H₂ (5 times). The resulting mixture was stirred o.n. at RT under an atmosphere of dihydrogen. The mixture was concentrated under reduced pressure. The residue was purified by silica gel column chromatography, eluting with EtOAc 0-60% in PE to afford 1-(tert-butyl) 2-methyl (2R,4R)-4-(2-(tert-butoxy)-2-oxoethyl)pyrrolidine-1,2-dicarboxylate. MS ESI calculated for C₁₇H₃₀NO₆ [M+H]⁺ 344.20, found 344.10.

Step 3: Chlorotrimethylsilane (18.6 mL, 146 mmol) and phenol (13.7 g, 146 mmol) were added to a mixture of 1-(tert-butyl) 2-methyl (2R,4R)-4-(2-(tert-butoxy)-2-oxoethyl)pyrrolidine-1,2-dicarboxylate (5.00 g, 14.6 mmol) in DCM (50 mL) at 0° C. The mixture was stirred at 0° C. for 1 h. The mixture was quenched with aq. 1 N LiOH (50 mL, 50 mmol) to pH ˜8-9 and then extracted with DCM (3×300 mL). The combined organic layers were washed with brine (3×100 mL), dried over anh. Na₂SO₄, filtered, and concentrated under reduced pressure to afford methyl (2R,4R)-4-(2-(tert-butoxy)-2-oxoethyl)pyrrolidine-2-carboxylate, which was used in the next step without purification. MS ESI calculated for C₁₂H₂₂NO₄ [M+H]⁺ 244.15, found 244.10.

Step 4: An aq. 1 N LiOH solution (29 mL, 29 mmol) was added to a solution of methyl (2R,4R)-4-(2-(tert-butoxy)-2-oxoethyl)pyrrolidine-2-carboxylate (3.54 g, 14.6 mmol) in THF (40 mL) at 0° C. The mixture was stirred at RT for 4 h. The mixture was acidified with an aq. 1 M HCl solution (30 mL) to pH ˜7 and then concentrated under reduced pressure to afford (2R,4R)-4-(2-(tert-butoxy)-2-oxoethyl)pyrrolidine-2-carboxylic acid, which was used in the next step without purification. MS ESI calculated for C₁₁H₂₀NO₄ [M+H]⁺ 230.13, found 230.10.

Step 5: Fmoc-OSu (4.37 g, 13.0 mmol) and NaHCO₃ (6.05 g, 72.0 mmol) were added to a solution of (2R,4R)-4-(2-(tert-butoxy)-2-oxoethyl)pyrrolidine-2-carboxylic acid (3.3 g, 14 mmol) in THF (40 mL) and water (40 mL) at 0° C. The mixture was stirred o.n. at RT. The mixture was acidified with an aq. 1 M HCl solution (30 mL) to pH ˜3 and extracted with EtOAc (3×200 mL). The combined organic phases were washed with brine (2×50 mL), dried over anh. Na₂SO₄, filtered, and concentrated under reduced pressure. The residue was purified by Rp-flash column chromatography with the following conditions: Column: Flash C18 (330 g); Mobile Phase A: water (0.1% TFA), Mobile Phase B: MeCN; Gradient Elution: 30-80%; Detector: UV 210 nm. The fractions containing the product were concentrated under reduced pressure to afford (2R,4S)-1-(((9H-fluoren-9-yl)methoxy)carbonyl)-4-(2-(tert-butoxy)-2-oxoethyl)pyrrolidine-2-carboxylic acid. The mixture of diastereomers was separated by SFC: (column: Daicel ChiralPak® IG (250×30 mm, 5 μm, Daicel Chiral Technologies, West Chester, PA); mobile phase: [1:1 MeOH in MeCN]; B %: 35-35%, 5 min) to afford (2R,4S)-1-(((9H-fluoren-9-yl) methoxy)carbonyl)-4-(2-(tert-butoxy)-2-oxoethyl)pyrrolidine-2-carboxylic acid as the second eluting isomer [t_{R}=3.27 min]. MS ESI calculated for C₂₆H₃₀NO₆ [M+H]⁺ 452.20, found 452.20. ¹H NMR (300 MHz, methanol-d₄): δ 7.81-7.77 (m, 2H), 7.65-7.60 (m, 2H), 7.41-7.28 (m, 4H), 4.35-4.17 (m, 4H), 3.79-3.73 (m, 1H), 3.11-3.06 (m, 1H), 2.57-2.50 (m, 2H), 2.40-2.35 (m, 2H), 1.70-1.68 (m, 1H), 1.46-1.45 (m, 9H).

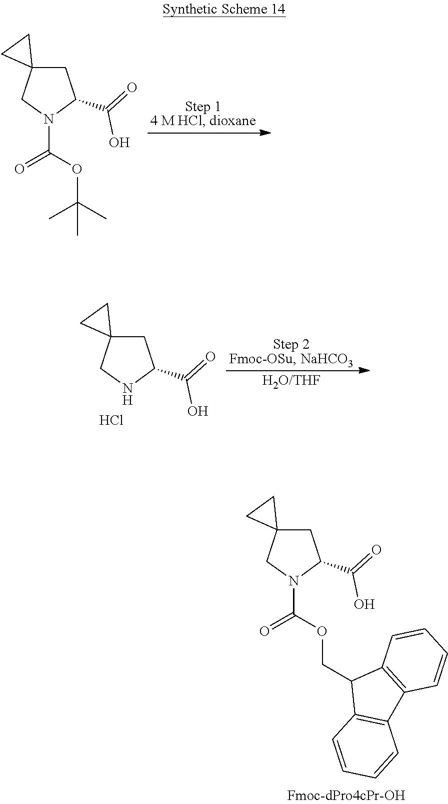 [SMILES: CC(C)(C)OC(=O)N1CC2(CC2)C[C@@H]1C(=O)O ; O=C(O)[C@H]1CC2(CC2)CN1 ; Cl ; O=C(O)[C@H]1CC2(CC2)CN1C(=O)OCC1c2ccccc2-c2ccccc21]

### (R)-5-(((9H-Fluoren-9-yl)methoxy)carbonyl)-5-azaspiro[2.4]heptane-6-carboxylic acid (Fmoc-dPro4cPr-OH)

Step 1: A solution of HCl in anh. dioxane (4 M, 2.6 mL, 10 mmol) was added to (R)-5-(tert-butoxycarbonyl)-5-azaspiro[2.4]heptane-6-carboxylic acid (0.25 g, 1.0 mmol). The resulting solution was stirred at RT for 4 h. The solution was then concentrated under reduced pressure. The crude product was used directly in the next step without any purification.

Step 2: To a stirred solution of the crude residue in water (5 mL) was added a suspension of Fmoc-OSu (0.35 g, 1.0 mmol) in THF (2.5 mL) followed by NaHCO₃ (0.26 g, 3.1 mmol) until pH >7. The resulting suspension was stirred at RT for 18 h. The organic volatiles were then removed under reduced pressure and aq. 0.2 M ammonium bicarbonate (5 mL) was added. The mixture was extracted with EtOAc (15 mL, discarded) then acidified to pH ˜2 by addition of conc. HCl (400 μL) and extracted with EtOAc (2×15 mL). The combined organic layers were dried over anh. Na₂SO₄, filtered and concentrated under reduced pressure. The residue was purified by Rp-flash column chromatography with the following conditions: Column: Flash C18 (30 g); Mobile Phase A: water (0.1% TFA), Mobile Phase B: MeCN (0.1% TFA); Gradient Elution: 20-60% over 16 column volumes; Detector: UV 215 nm. The product-containing fractions were combined, concentrated under reduced pressure and freeze-dried to afford (R)-5-(((9H-fluoren-9-yl)methoxy)carbonyl)-5-azaspiro[2.4]heptane-6-carboxylic acid. MS ESI calculated for C₂₂H₂₁NO₄ [M+H]⁺ 364.15, found 364.0. ¹H NMR (DMSO-d₆): δ 7.90 (d, J=7.4 Hz, 2H), 7.69-7.63 (m, 2H), 7.46-7.40 (m, 2H), 7.38-7.29 (m, 2H), 4.47-4.16 (m, 4H), 3.44-3.39 (m, 1H), 3.32-3.21 (m, 1H), 2.46-2.28 (m, 1H), 1.82-1.71 (m, 1H), 0.64-0.49 (m, 4H).

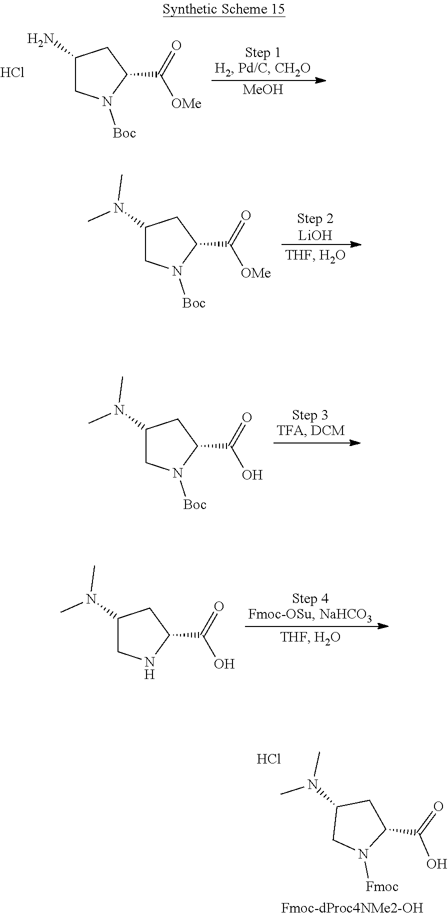

### (2R,4R)-1-(((9H-Fluoren-9-yl)methoxy)carbonyl)-4-(dimethylamino)pyrrolidine-2-carboxylic acid hydrochloride (Fmoc-dProc4NMe2-OH)

Step 1: DIPEA (1.77 g, 13.7 mmol) and Pd/C (300 mg, 0.28 mmol, dry, 10 wt. %) were added to a solution of 1-(tert-butyl) 2-methyl (2R,4R)-4-aminopyrrolidine-1,2-dicarboxylate hydrochloride (3.50 g, 12.5 mmol) in MeOH (65 mL). Formaldehyde (5.5 mL, 12 mmol, 37 wt. % in water) was added dropwise to the reaction mixture and the mixture was degassed with hydrogen (3 times). The mixture was stirred under hydrogen o.n. at RT under 1 atm. The mixture was filtered through a pad of Celite®. The filtered cake was washed with MeOH (3×200 mL). The filtrate was concentrated under vacuum. The residue was purified by silica gel column chromatography, eluting with MeOH 10% in DCM to afford 1-(tert-butyl) 2-methyl (2R,4R)-4-(dimethylamino) pyrrolidine-1,2-dicarboxylate. MS ESI calculated for C₁₃H₂₅N₂O₄ [M-Boc+H]⁺ 173.17, found 173.10.

Step 2: LiOH (36 mL, 36 mmol, 1 N in water) was added to a mixture of 1-(tert-butyl) 2-methyl (2R,4R)-4-(dimethylamino) pyrrolidine-1,2-dicarboxylate (3.0 g, 11 mmol) in THF (36 mL). The resulting mixture was stirred for 2 h at RT. The mixture was acidified to pH 5 with aq. 1 N HCl. The filtrate was concentrated under vacuum to afford the crude product. The crude product was directly used into the next step.

Step 3: TFA (11 mL, 140 mmol) at RT was added to a stirred mixture of (2R,4R)-1-(tert-butoxycarbonyl)-4-(dimethylamino) pyrrolidine-2-carboxylic acid (2.8 g, 11 mmol) in DCM (33 mL). The resulting mixture was stirred for 1 h at RT. The solvent was evaporated under reduced pressure to give crude (2R,4R)-4-(dimethylamino) pyrrolidine-2-carboxylic acid. MS ESI calculated for C₇H₁₅N₂O₂ [M+H]⁺ 159.11, found 159.20.

Step 4: NaHCO₃ (0.96 g, 11 mmol) and Fmoc-OSu (3.84 g, 11.4 mmol) at RT were added to a stirred solution of (2R,4R)-4-(dimethylamino) pyrrolidine-2-carboxylic acid (1.8 g, 11 mmol) in water (18 mL) and THF (18 mL). The resulting mixture was stirred at RT for 4 h. The pH was adjusted to 3 with 1 N HCl. The solvent was concentrated under reduced pressure and the residue was purified by Rp-flash column chromatography with the following conditions: 330 g C18 column, 2%-2% in 5 min, 2%-60% in 30 min, MeCN in water (0.05% TFA) to give (2R,4R)-1-(((9H-fluoren-9-yl) methoxy) carbonyl)-4-(dimethylamino) pyrrolidine-2-carboxylic acid. MS ESI calculated for C₂₂H₂₅N₂O₄ [M−HCl+H]⁺ 381.17, found 381.10. ¹H NMR (300 MHz, DMSO-d₆): δ 7.92-7.86 (m, 2H), 7.69-7.63 (m, 2H), 7.45-7.32 (m, 4H), 4.45-4.29 (m, 2H), 4.17-4.11 (m, 2H), 3.91-3.78 (m, 1H), 3.35-3.26 (m, 2H), 2.80-2.74 (m, 4H), 2.62-2.47 (m, 3H), 2.03-1.86 (m, 1H).

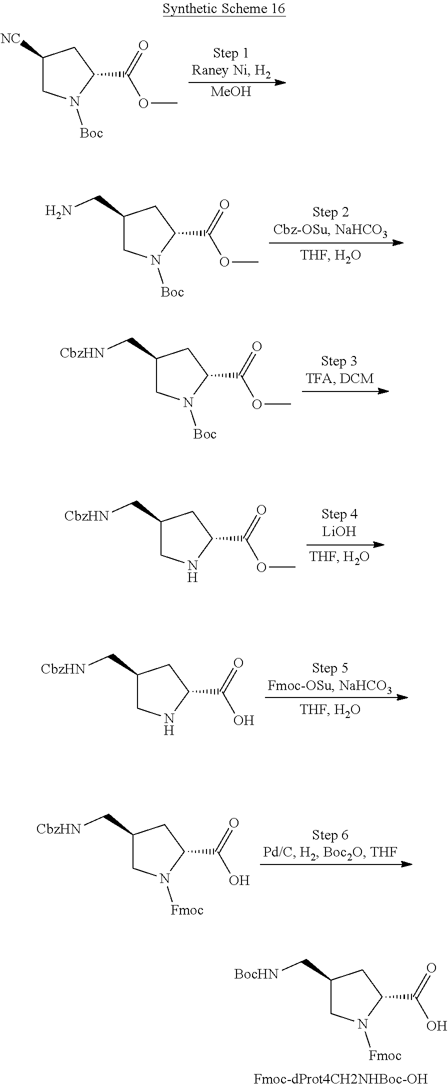

### (2R,4R)-1-(((9H-Fluoren-9-yl)methoxy)carbonyl)-4-(((tert-butoxycarbonyl)amino)-methyl)pyrrolidine-2-carboxylic acid (Fmoc-dProt4CH2NHBoc-OH)

Step 1: A solution of 1-(tert-butyl) 2-methyl (2R,4S)-4-cyanopyrrolidine-1,2-dicarboxylate (1.1 g, 4.3 mmol) in methanol (20 mL) was degassed with N₂. Raney®-Nickel (50 mg, 0.85 mmol) was added to the solution under N₂. The mixture was then stirred under hydrogen (1 atm) for 6 h at RT. The mixture was filtered through Celite®, washed with methanol (2×30 mL). The filtrate was concentrated under reduced pressure to afford 1-(tert-butyl) 2-methyl (2R,4R)-4-(aminomethyl)pyrrolidine-1,2-dicarboxylate. MS ESI calculated for C₁₂H₂₃N₂O₄-Boc [M−Boc+H]⁺ 159.16, found 159.15.

Step 2: NaHCO₃ (1.62 g, 19.3 mmol) and Cbz-OSu (1.16 g, 4.63 mmol) were added to a solution of 1-(tert-butyl) 2-methyl (2R,4R)-4-(aminomethyl)pyrrolidine-1,2-dicarboxylate (1.05 g, 3.86 mmol) in THF (20 mL) and water (20 mL) at RT. The mixture was stirred at RT for 4 h. The mixture was diluted with water (20 mL) and extracted with EtOAc (3×50 mL). The combined organic layers were washed with brine (2×20 mL), dried over anh. Na₂SO₄, filtered, and concentrated under reduced pressure. The residue was purified by silica gel chromatography eluting with EtOAc in PE to afford 1-(tert-butyl) 2-methyl (2R,4R)-4-((((benzyloxy)carbonyl)amino)methyl)pyrrolidine-1,2-dicarboxylate. MS ESI calculated for C₂₀H₂₉N₂O₆ [M+H]⁺ 393.19, found 393.05.

Step 3: TFA (5.0 mL, 65 mmol) was added to a solution of 1-(tert-butyl) 2-methyl (2R,4R)-4-((((benzyloxy)carbonyl)amino)methyl)pyrrolidine-1,2-dicarboxylate (1.4 g, 2.5 mmol) in DCM (10 mL) at RT. The mixture was stirred at RT for 2 h. The mixture was concentrated under reduced pressure to afford methyl (2R,4S)-4-((((benzyloxy)carbonyl)amino)methyl) pyrrolidine-2-carboxylate, which was used in the next step without purification. MS ESI calculated for C₁₅H₂₁N₂O₄ [M+H]⁺ 293.14, found 293.10.

Step 4: An aq. 1 N solution of LiOH (4.9 mL, 4.9 mmol) was added to a solution of methyl (2R,4S)-4-((((benzyloxy)carbonyl)amino)methyl)pyrrolidine-2-carboxylate (1.2 g, 2.5 mmol) in THF (10 mL) at RT. The mixture was stirred at RT for 2 h. The mixture was then acidified to pH ˜5 with an aq. 1 M HCl solution. The mixture was concentrated under reduced pressure to afford (2R,4S)-4-((((benzyloxy)carbonyl)amino)methyl)pyrrolidine-2-carboxylic acid, which was used in the next step without purification. MS ESI calculated for C₁₄H₁₉N₂O₄+MeCN [M+MeCN+H]⁺ 320.13, found 319.75.

Step 5: NaHCO₃(0.996 g, 11.9 mmol) and Fmoc-OSu (0.720 g, 2.13 mmol) were added to a solution of (2R,4S)-4-((((benzyloxy)carbonyl)amino)methyl)pyrrolidine-2-carboxylic acid (1.2 g, 2.4 mmol) in THF (20 mL) and water (20 mL) at RT. The mixture was stirred at RT for 2 h. The mixture was then acidified to pH ˜5 with an aq. 1 M HCl solution and then concentrated under reduced pressure. The residue was purified by Rp-flash column chromatography with the following conditions: Column: Flash C18 (80 g); Mobile Phase A: water (0.1% TFA), Mobile Phase B: MeCN; Gradient Elution: 2-40%; Detector: UV 210 nm. The fractions containing the product were concentrated under reduced pressure to afford (2R,4R)-1-(((9H-fluoren-9-yl)methoxy)carbonyl)-4-((((benzyloxy)carbonyl)amino)methyl)pyrrolidine-2-carboxylic acid. MS ESI calculated for C₂₉H₂₉N₂O₆ [M+H]⁺ 501.19, found 501.10.

Step 6: (2R,4R)-1-(((9H-Fluoren-9-yl)methoxy)carbonyl)-4-((((benzyloxy)carbonyl) amino)methyl)pyrrolidine-2-carboxylic acid (820 mg, 1.64 mmol) and Boc₂O (536 mg, 2.46 mmol) were dissolved in THF (15 mL). The flask was evacuated and refilled with N₂ (5 times). Pd/C (50 mg, 0.5 mmol, dry, 10 wt. %) was added into the flask. The flask was evacuated and backfilled with H₂ (5 times). The resulting mixture was stirred for 6 h at RT under an atmosphere of dihydrogen. The flask was evacuated and refilled with N₂ (5 times). The resulting mixture was filtered through a Celite® pad. The filtered cake was washed with THF (2×30 mL). The filtrate was concentrated under reduced pressure. The residue was purified by Rp-flash column chromatography with the following conditions: Column: Flash C18 (80 g); Mobile Phase A: water (0.1% TFA), Mobile Phase B: MeCN; Gradient Elution: 2-40%; Detector: UV 210 nm. The fractions containing the product were concentrated under reduced pressure to afford (2R,4R)-1-(((9H-fluoren-9-yl)methoxy)carbonyl)-4-(((tert-butoxycarbonyl)amino)methyl)pyrrolidine-2-carboxylic acid. MS ESI calculated for C₂₆H₃₄N₃O₆ [M+NH₄]⁺ 484.21, found 484.25. ¹H NMR (300 MHz, methanol-d₄): δ 7.85-7.74 (m, 2H), 7.71-7.57 (m, 2H), 7.48-7.23 (m, 4H), 4.57-4.12 (m, 4H), 3.74-3.57 (m, 1H), 3.25-3.13 (m, 1H), 3.13-2.99 (m, 2H), 2.64-2.41 (m, 1H), 2.15-1.96 (m, 2H), 1.51-1.29 (m, 9H).

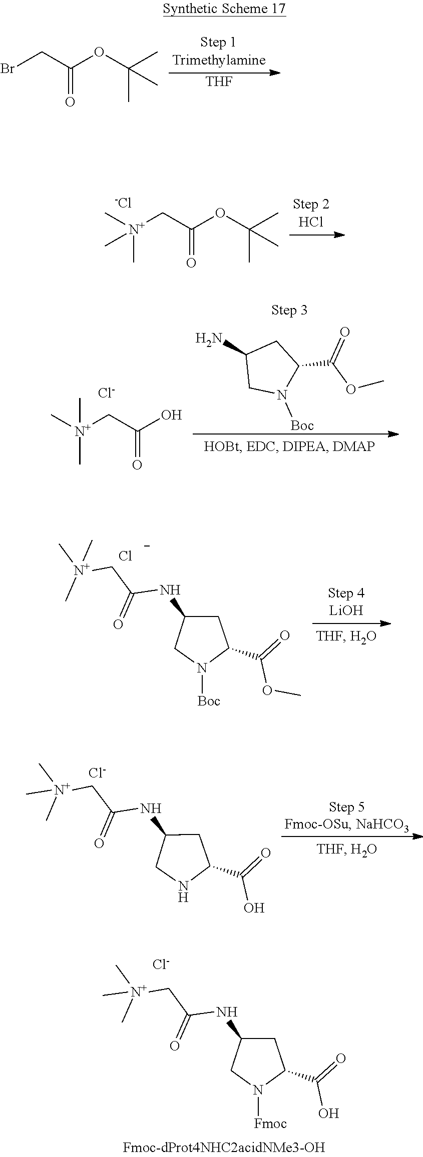

### 2-(((3S,5R)-1-(((9H-Fluoren-9-yl)methoxy)carbonyl)-5-carboxypyrrolidin-3-yl)amino)-N,N,N-trimethyl-2-oxoethan-1-aminium chloride (Fmoc-dProt4NHC2acidNMe3-OH)

Step 1: A mixture of tert-butyl 2-bromoacetate (1.9 g, 9.7 mmol) in a solution of trimethylamine (2 N in THF, 10 mL, 20 mmol) was stirred and heated o.n. at 50° C. The mixture was cooled to RT and concentrated under reduced pressure to afford 2-(tert-butoxy)-N,N,N-trimethyl-2-oxoethan-1-aminium chloride, which was used in the next step without purification. MS ESI calculated for C₉H₂₀NO₂⁺ [M]⁺ 174.15, found 174.10.

Step 2: A mixture of 2-(tert-butoxy)-N,N,N-trimethyl-2-oxoethan-1-aminium chloride (2.0 g, 9.5 mmol) in a solution of hydrochloric acid (4.0 M in 1,4-dioxane, 24 mL, 95 mmol) was stirred at RT for 4 h. The mixture was concentrated under reduced pressure to afford 1-carboxy-N,N,N-trimethylmethanaminium chloride, which was used in the next step without purification. MS ESI calculated for C₅H₁₂NO₂+[M]⁺ 118.09, found 118.05.

Step 3: A mixture of 1-carboxy-N,N,N-trimethylmethanaminium chloride (3.3 g, 21 mmol), HOBt (4.94 g, 32.2 mmol), DIPEA (7.50 mL, 43.0 mmol), EDC (6.18 g, 32.2 mmol), DMAP (0.262 g, 2.15 mmol), and 1-(tert-butyl) 2-methyl (2R,4S)-4-aminopyrrolidine-1,2-dicarboxylate (5.25 g, 21.5 mmol) in DMSO (33 mL) was stirred o.n. at RT. The residue was purified by Rp-flash column chromatography with the following conditions: Column: Flash C18; Mobile Phase A: water (0.1% HCl), Mobile Phase B: MeCN; Gradient Elution: 5-90%; Detector: UV 210 nm. The fractions containing the product were concentrated under reduced pressure to afford 2-(((3S,5R)-1-(tert-butoxycarbonyl)-5-(methoxycarbonyl)pyrrolidin-3-yl)amino)-N,N,N-trimethyl-2-oxoethan-1-aminium chloride. MS ESI calculated for C₁₆H₃₀N₃O₅⁺ [M]⁺ 344.22, found 344.05.

Step 4: An aq. 1 N LiOH solution (20.3 mL, 20 mmol) was added to a mixture of 2-(((3S,5R)-1-(tert-butoxycarbonyl)-5-(methoxycarbonyl)pyrrolidin-3-yl)amino)-N,N,N-trimethyl-2-oxoethan-1-aminium chloride (3.5 g, 10 mmol) in THF (35 mL) at RT. The mixture was stirred at RT for 4 h. The mixture was acidified with aq. 1 M HCl solution to pH ˜2-3 to afford 2-(((3S,5R)-5-carboxypyrrolidin-3-yl)amino)-N,N,N-trimethyl-2-oxoethan-1-aminium chloride, which was used directly in next step. MS ESI calculated for C₁₀H₂₀N₃O₃⁺ [M]⁺ 230.15, found 230.15.

Step 5: Fmoc-OSu (3.56 g, 10.6 mmol) and NaHCO₃ (4.92 g, 58.6 mmol) were added to a solution of 2-(((3S,5R)-5-carboxypyrrolidin-3-yl)amino)-N,N,N-trimethyl-2-oxoethan-1-aminium chloride (2.7 g, 12 mmol) in THF (30 mL) and water (30 mL) at RT. The mixture was stirred o.n. at RT. The pH of the mixture was adjusted to pH ˜3 with 1 M HCl and concentrated under reduced pressure. The residue was purified by Rp-flash column chromatography with the following conditions: Column: Flash C18; Mobile Phase A: water (0.1% HCl), Mobile Phase B: MeCN; Gradient Elution: 5-70%; Detector: UV 210 nm. The fractions containing the product were concentrated under reduced pressure to afford 2-(((3S,5R)-1-(((9H-fluoren-9-yl)methoxy) carbonyl)-5-carboxypyrrolidin-3-yl)amino)-N,N,N-trimethyl-2-oxoethan-1-aminium chloride. MS ESI calculated for C₂₅H₃₀N₃O₅⁺ [M]⁺ 452.22, found 452.15. ¹H NMR (400 MHz, methanol-d₄): δ 7.91-7.75 (m, 2H), 7.67-7.63 (m, 2H), 7.50-7.18 (m, 4H), 4.60-4.02 (m, 7H), 3.86-3.79 (m, 1H), 3.43-3.41 (m, 2H), 3.34 (s, 8H), 2.40-2.31 (m, 2H).

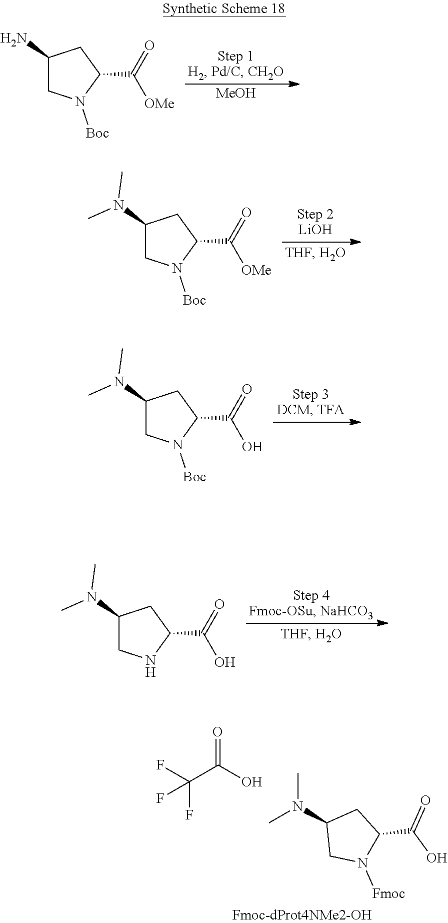

### (2R,4S)-1-(((9H-Fluoren-9-yl)methoxy)carbonyl)-4-(dimethylamino)pyrrolidine-2-carboxylic acid trifluoroacetic acid (Fmoc-dProt4NMe2-OH)

Step 1: Pd/C (1.089 g, 1.0 mmol, 10 wt. %) was added to a solution of 1-(tert-butyl) 2-methyl (2R,4S)-4-aminopyrrolidine-1,2-dicarboxylate (2.5 g, 10 mmol) in MeOH (30 mL). Then formaldehyde (2.5 mL, 10 mmol, 37 wt. % in water) was added under N₂. The reaction mixture was degassed with dihydrogen (3 times) and stirred under hydrogen o.n. at RT under 1 atm. The mixture was filtered through a pad of Celite®. The filtered cake was further washed with MeOH (3×50 mL). The filtrate was concentrated under vacuum. The residue was purified by silica gel column chromatography, eluting with MeOH 0-10% in DCM to afford 1-(tert-butyl) 2-methyl (2R,4S)-4-(dimethylamino) pyrrolidine-1,2-dicarboxylate. MS ESI calculated for C₁₃H₂₅N₂O₄ [M+H]⁺ 273.17, found 272.95. ¹H NMR (300 MHz, chloroform-d): δ 4.49-4.21 (m, 1H), 3.86-3.83 (m, 1H), 3.74 (s, 3H), 3.25-3.22 (m, 1H), 2.92-2.78 (m, 1H), 2.25 (s, 6H), 2.18-1.98 (m, 2H), 1.44 (m, 9H).

Step 2: LiOH (15 mL, 15 mmol, 1 N in water) was added to a mixture of 1-(tert-butyl) 2-methyl (2R,4S)-4-(dimethylamino) pyrrolidine-1,2-dicarboxylate (1.7 g, 6.2 mmol) in THF (15 mL). The resulting mixture was stirred for 2 h at RT. The mixture was acidified to pH 5 with 1 N HCl solution. The solvent was concentrated under vacuum to afford the crude product, which was directly used in the next step.

Step 3: TFA (10 mL, 5.4 mmol) at RT was added to a stirred mixture of (2R,4S)-1-(tert-butoxycarbonyl)-4-(dimethylamino) pyrrolidine-2-carboxylic acid (1.4 g, 5.4 mmol) in DCM (30 mL). The resulting mixture was stirred for 1 h at RT. The solvent was evaporated under reduced pressure to give crude (2R,4S)-4-(dimethylamino) pyrrolidine-2-carboxylic acid. MS ESI calculated for C₇H₁₃N₂O₂ [M−H]⁻ 157.11, found 157.10.

Step 4: NaHCO₃ (2.12 g, 25.3 mmol) and Fmoc-OSu (1.71 g, 5.06 mmol) at RT were added to a stirred solution of (2R,4S)-4-(dimethylamino) pyrrolidine-2-carboxylic acid (800 mg, 5.06 mmol) in THF (15 mL) and water (15 mL). The resulting mixture was stirred o.n. at RT. The pH was adjusted to 3 with 1 N HCl. The solvent was concentrated under reduced pressure and the residue was purified by Rp-flash column chromatography with the following conditions: 330 g C18 column, 2-2% in 5 min, 2-50% in 30 min, MeCN in water (0.05% TFA) to give (3S,5R)-1-(((9H-fluoren-9-yl) methoxy) carbonyl)-5-carboxy-N,N-dimethylpyrrolidin-3-aminium 2,2,2-trifluoroacetate. MS ESI calculated for C₂₅H₂₅N₂O₄ [M+H]⁺ 381.17, found 381.10. ¹H NMR (300 MHz, DMSO-d₆): δ 10.53 (s, 1H), 7.92-7.86 (m, 2H), 7.69-7.63 (m, 2H), 7.45-7.32 (m, 4H), 4.56-4.53 (m, 1H), 4.41-4.29 (m, 3H), 4.17-3.89 (m, 2H), 3.83-3.62 (m, 1H), 2.83-2.80 (s, 6H), 2.62-2.47 (m, 1H), 2.40-2.36 (m, 1H). ¹⁹F NMR (282 MHz, DMSO-d₆): δ −73.73.

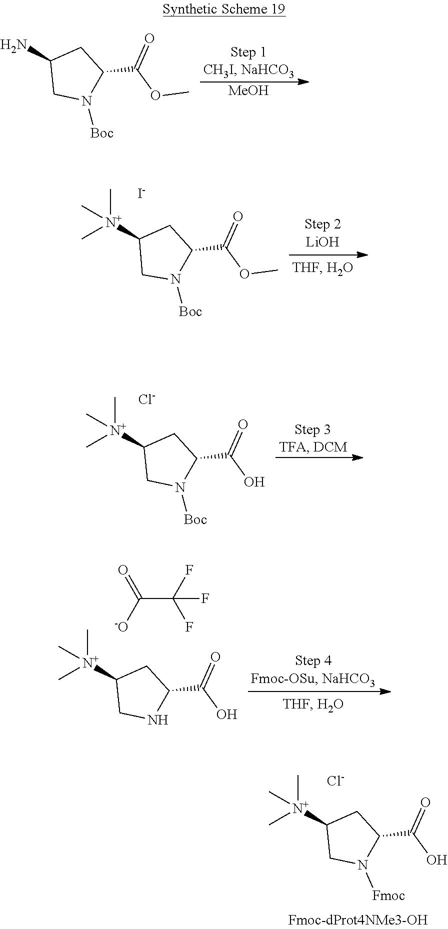

### (3S,5R)-1-(((9H-Fluoren-9-yl)methoxy)carbonyl)-5-carboxy-N,N,N-trimethylpyrrolidin-3-aminium chloride (Fmoc-dProt4NMe3-OH)

Step 1: NaHCO₃(8.3 g, 98 mmol) and iodomethane (4.6 mL, 73.7 mmol) were added to a solution of 1-(tert-butyl) 2-methyl (2R,4S)-4-aminopyrrolidine-1,2-dicarboxylate (3.0 g, 12.3 mmol) in MeOH (30 mL) at RT. The mixture was stirred o.n. at RT. The mixture was filtered, and the filtrate was concentrated under reduced pressure. The residue was dissolved in DCM and filtered, and the filtrate was concentrated under reduced pressure to afford (3S,5R)-1-(tert-butoxycarbonyl)-5-(methoxycarbonyl)-N,N,N-trimethylpyrrolidin-3-aminium iodide, which was used in Step 2 without purification. MS ESI calculated for C₁₄H₂₇N₂O₄ [M]⁺ 287.20, found 287.15.

Step 2: An aq. 1 N LiOH solution (22 mL, 22 mmol) was added to a solution of (3S,5R)-1-(tert-butoxycarbonyl)-5-(methoxycarbonyl)-N,N,N-trimethylpyrrolidin-3-aminium iodide (6.5 g, 11 mmol) in THF (23 mL) at RT. The mixture was stirred at RT for 4 h. The mixture was acidified to pH ˜5 with aq. 1 M HCl, and then concentrated under reduced pressure to afford (3S,5R)-1-(tert-butoxycarbonyl)-5-carboxy-N,N,N-trimethylpyrrolidin-3-aminium chloride, which was used in Step 3 without purification. MS ESI calculated for C₁₃H₂₅N₂O₄ [M]⁺ 273.18, found 273.15.

Step 3: TFA (20 mL, 260 mmol) was added to a mixture of (3S,5R)-1-(tert-butoxycarbonyl)-5-carboxy-N,N,N-trimethylpyrrolidin-3-aminium chloride (6.6 g, 11 mmol) in DCM (20 mL) at RT. The mixture was stirred at RT for 2 h. The mixture was concentrated under reduced pressure to afford 2,2,2-trifluoroacetic acid, (3S,5R)-1-(tert-butoxycarbonyl)-5-carboxy-N,N,N-trimethylpyrrolidin-3-aminium salt, which was used without purification in Step 4. MS ESI calculated for C₈H₁₇N₂O₂ [M]⁺ 173.13, found 173.25.

Step 4: NaHCO₃(5.92 g, 70.5 mmol) and Fmoc-OSu (4.28 g, 12.7 mmol) were added to a mixture of (3S,5R)-5-carboxy-N,N,N-trimethylpyrrolidin-3-aminium trifluoroacetate salt (8.1 g, 14 mmol) in THF (40 mL) and water (40 mL) at RT. The mixture was stirred at RT for 4 h. The mixture was acidified to pH ˜5 with an aq. 1 N HCl solution and then concentrated under reduced pressure. The residue was suspended in a 9:1 (v/v) mixture of DCM and MeOH and the solids were removed by filtration. The filtrate was concentrated under reduced pressure, The residue was purified by Rp-flash column chromatography with the following conditions: Column: Flash C18 (330 g); Mobile Phase A: water (2 mM HCl), Mobile Phase B: MeCN; Gradient Elution: 2-35%; Detector: UV 210 nm. The fractions containing the product were concentrated under reduced pressure to afford (3S,5R)-1-(((9H-fluoren-9-yl)methoxy)carbonyl)-5-carboxy-N,N,N-trimethylpyrrolidin-3-aminium chloride. MS ESI calculated for C₂₃H₂₇N₂O₄ [M]⁺ 395.20, found 395.20. ¹H NMR (300 MHz, DMSO-d₆): δ 13.30 (s, 1H), 7.94-7.89 (m, 2H), 7.72-7.64 (m, 2H), 7.46-7.33 (m, 4H), 4.57-4.14 (m, 5H), 3.86-3.78 (m, 2H), 3.12-3.10 (m, 9H), 2.83-2.73 (m, 1H), 2.50-2.35 (m, 1H).

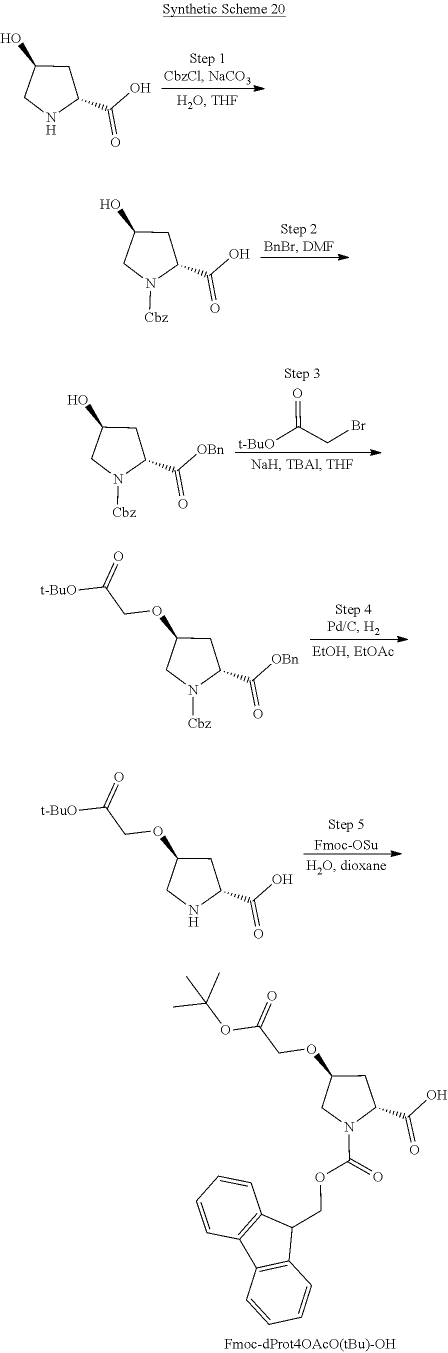

### (2R,4S)-1-(((9H-Fluoren-9-yl)methoxy)carbonyl)-4-(2-(tert-butoxy)-2-oxoethoxy)pyrrolidine-2-carboxylic acid (Fmoc-dProt4OAcO(tBu)-OH)

Step 1: NaHCO₃ (22.9 g, 0.27 mmol) and CbzCl (24.3 g, 0.14 mmol) were added to a stirred solution of (2R,4S)-4-hydroxypyrrolidine-2-carboxylic acid in (15.6 g, 0.12 mol) in DI water (75 mL) and THF (30 mL). The reaction mixture was stirred at RT for 12 h. The reaction solution was diluted in DI water (100 mL) and extracted with MTBE (2×150 mL). The pH of the aqueous solution was adjusted to pH ˜2 by addition of aq. 1 N HCl solution. The solution was extracted with EtOAc (2×150 mL). The combined organic phases were washed with brine (50 mL), dried over anh. Na₂SO₄, and concentrated in vacuum to afford (2R,4S)-1-((benzyloxy) carbonyl)-4-hydroxypyrrolidine-2-carboxylic acid. The crude product was used directly in Step 2 without further purification. ¹H NMR (400 MHz, chloroform-d): δ 7.28 (s, 5H), 6.82-6.38 (m, 1H), 5.16-5.11 (m, 2H), 4.70-4.44 (m, 2H), 4.16-4.11 (m, 1H), 3.63-3.57 (m, 2H), 2.34-2.06 (m, 3H), 1.27 (d, J=12 Hz, 1H).

Step 2: To a solution of (2R,4S)-1-((benzyloxy)carbonyl)-4-hydroxypyrrolidine-2-carboxylic acid (36.0 g, 136 mmol) in anh. DMF (240 mL) was added NaHCO₃ (34.2 g, 407 mmol), NaI (2.03 g, 13.6 mmol), and BnBr (19.3 mL, 162 mmol). The mixture was stirred at RT for 12 h. The reaction solution was diluted in DI water (100 mL) and extracted with EtOAc (2×150 mL). The combined organic phases were washed with brine (50 mL), dried over anh. Na₂SO₄ and concentrated in vacuum. The residue was purified by silica gel column chromatography, eluting with EtOAc 0-60% in PE to give dibenzyl (2R,4S)-4-hydroxypyrrolidine-1,2-dicarboxylate. ¹H NMR (400 MHz, chloroform-d): δ 7.21 (m, 10H), 5.12-5.05 (m, 2H), 4.94-4.91 (m, 2H), 4.49-4.36 (m, 2H), 3.57-3.56 (m, 2H), 2.42 (m, 1H), 2.01-1.95 (m, 1H).

Step 3: NaH (2.45 g, 61 mmol, 60 wt. % dispersion in mineral oil) was added to a solution of tert-butyl 2-bromoacetate (22.3 mL, 150 mmol) and TBAI (1.51 g, 4.08 mmol) in anh. THF (30 mL). The suspension was cooled at −10° C. A solution of (2R,4S)-1-((benzyloxy) carbonyl)-4-hydroxypyrrolidine-2-carboxylic acid (14.5 g, 40.8 mmol) in anh. THF (30 mL) was added dropwise to the mixture maintained below −10° C. After addition, the reaction mixture was allowed to warm to RT and was stirred at RT for 12 h. The reaction mixture was slowly poured into iced sat. NH₄Cl (75 mL) and extracted with EtOAc (2×100 mL). The combined organic layers were washed with brine (50 mL), dried over anh. Na₂SO₄, filtered, and concentrated in vacuum. The residue was purified by silica gel column chromatography, eluting with EtOAc 1-50% in PE to give dibenzyl (2R,4S)-4-(2-(tert-butoxy)-2-oxoethoxy)pyrrolidine-1,2-dicarboxylate. ¹H NMR (400 MHz, chloroform-d): δ 7.32-7.15 (m, 10H), 5.16-4.97 (m, 4H), 4.44-4.41 (m, 1H), 3.75-3.68 (m, 1H), 3.64-3.59 (m, 1H), 2.33-2.19 (m, 2H), 1.41 (s, 9H).

Step 4: A solution of dibenzyl (2R,4S)-4-(2-(tert-butoxy)-2-oxoethoxy)pyrrolidine-1,2-dicarboxylate (18.0 g, 48.9 mmol) in a 1:1 (v/v) mixture of EtOAc and EtOH (460 mL) was hydrogenated over Pd/C (1.0 g, 10 wt. %) at 50° C. for 24 h under 50 psi of dihydrogen. The reaction mixture was filtered through a Celite® pad, and the filtrate was concentrated to give the crude product (2R,4S)-4-(2-(tert-butoxy)-2-oxoethoxy)pyrrolidine-2-carboxylic acid. MS ESI calculated for C₁₁H₁₉NO₅[M+H]⁺ 246.13, found 246.2. ¹H NMR (400 MHz, methanol-d₄): δ 4.32-4.29 (m, 1H), 4.17-4.12 (m, 1H), 4.06-4.04 (t, J=8 Hz, 2H), 3.46-3.36 (m, 1H), 2.55-2.49 (m, 1H), 2.06-1.99 (m, 1H), 1.47 (s, 9H).

Step 5: NaHCO₃(6.2 g, 73 mmol) and Fmoc-OSu (12.3 g, 36.5 mmol) were added to a solution of (2R,4S)-4-(2-(tert-butoxy)-2-oxoethoxy)pyrrolidine-2-carboxylic acid (9.0 g, 37 mmol) in dioxane (40 mL) and DI water (20 mL). The reaction mixture was stirred at RT for 5 h. The reaction solution was diluted in DI water (50 mL) and extracted with MTBE (2×30 mL). The pH was adjusted to 2 with aq. 1 N HCl solution and extracted with EtOAc (2×80 mL). The combined organic layers were washed with brine (50 mL), dried over anh. Na₂SO₄ and filtered. The filtrate was concentrated under reduced pressure and the residue was purified by silica gel column chromatography, eluting with EtOAc 1-100% in PE to afford (2R,4S)-1-(((9H-fluoren-9-yl)methoxy)carbonyl)-4-(2-(tert-butoxy)-2-oxoethoxy)pyrrolidine-2-carboxylic acid. ¹H NMR (400 MHz, chloroform-d): δ 7.69-7.67 (m, 2H), 7.62-7.60 (m, 2H), 7.51-7.43 (m, 4H), 4.66-4.33 (m, 3H), 4.18-4.15 (m, 2H), 3.88-3.83 (m, 2H), 3.63-3.51 (m, 2H), 2.35-2.30 (m, 1H), 2.24-2.08 (m, 1H), 1.41 (s, 9H).

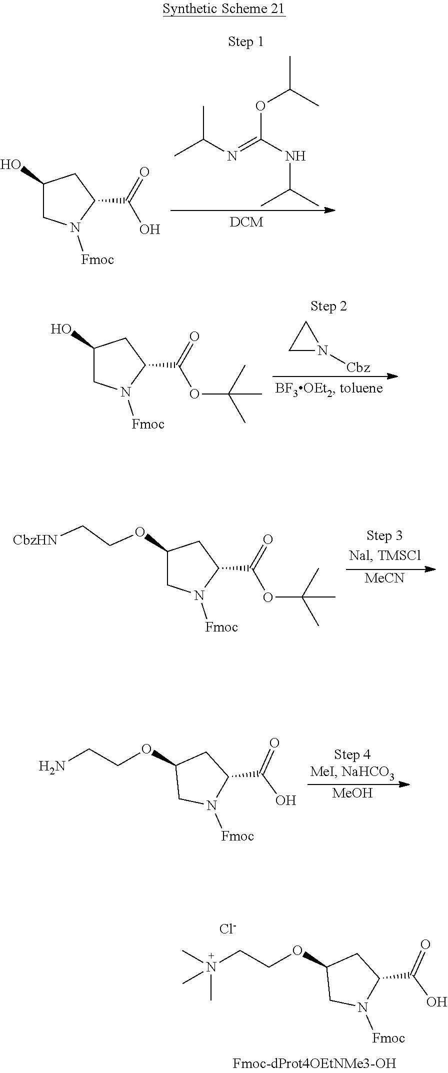

### 2-(((3S,5R)-1-(((9H-Fluoren-9-yl)methoxy)carbonyl)-5-carboxypyrrolidin-3-yl)oxy)-N,N,N-trimethylethan-1-aminium chloride (Fmoc-dProt4OEtNMe3-OH)

Step 1: tert-Butyl (E)-N,N′-diisopropylcarbamimidate (22.7 g, 113 mmol) was added to a solution of (2R,4S)-1-(((9H-fluoren-9-yl)methoxy)carbonyl)-4-hydroxypyrrolidine-2-carboxylic acid (10.0 g, 28.3 mmol) in DCM (100 mL) at RT. The mixture was stirred at 40° C. for 1 h. The mixture was cooled to RT and concentrated under reduced pressure. The residue was purified by silica gel column chromatography, eluting with EtOAc 0-70% in PE to afford 1-((9H-fluoren-9-yl)methyl) 2-(tert-butyl) (2R,4S)-4-hydroxypyrrolidine-1,2-dicarboxylate. MS ESI calculated for C₂₄H₂₇NO₅Na [M+Na]⁺ 432.19, found 432.05.

Step 2: Benzyl aziridine-1-carboxylate (4.85 g, 27.4 mmol) was added to a mixture of 1-((9H-fluoren-9-yl)methyl) 2-(tert-butyl) (2R,4S)-4-hydroxypyrrolidine-1,2-dicarboxylate (11.2 g, 27.4 mmol) in toluene (110 mL) at 0° C. The mixture was stirred at 0° C. for 30 min. Boron trifluoride diethyl etherate (0.776 g, 5.47 mmol) was added dropwise to the mixture at 0° C. over a period of 1 h. The mixture was stirred for an additional 1 h at RT. The mixture was concentrated under reduced pressure and the residue was purified by silica gel column chromatography, eluting with EtOAc 0-70% in PE to afford 1-((9H-fluoren-9-yl)methyl) 2-(tert-butyl) (2R,4S)-4-(2-(((benzyloxy)carbonyl)amino)ethoxy)pyrrolidine-1,2-dicarboxylate. MS ESI calculated for C₃₄H₃₈N₂O₇Na [M+Na]⁺ 609.27, found 609.50. ¹H NMR (400 MHz, methanol-d₄): δ 7.74-7.56 (m, 2H), 7.55-7.41 (m, 1H), 7.37-7.16 (m, 10H), 5.03 (s, 2H), 4.43-3.96 (m, 5H), 3.70-3.52 (m, 1H), 3.51-3.19 (m, 5H), 2.38-2.16 (m, 1H), 2.08-1.91 (m, 1H), 1.39 (s, 9H).

Step 3: Sodium iodide (4.85 g, 32.4 mmol) and chlorotrimethylsilane (3.52 g, 32.4 mmol) were added to a solution of 1-((9H-fluoren-9-yl)methyl) 2-(tert-butyl) (2R,4S)-4-(2-(((benzyloxy) carbonyl)amino)ethoxy)pyrrolidine-1,2-dicarboxylate (1.9 g, 3.2 mmol) in MeCN (20 mL) at RT. The mixture was stirred o.n. at RT. The mixture was concentrated under reduced pressure and the residue was purified by Rp-flash column chromatography with the following conditions: Column: Flash C18 (40 g); Mobile Phase A: water (0.05% TFA), Mobile Phase B: MeCN; Gradient Elution: 2-30%; Detector: UV 210 nm. The fractions containing the product were concentrated under reduced pressure to afford (2R,4S)-1-(((9H-fluoren-9-yl)methoxy)carbonyl)-4-(2-aminoethoxy)pyrrolidine-2-carboxylic acid. MS ESI calculated for C₂₂H₂₅N₂O₅[M+H]⁺ 397.17, found 397.40.

Step 4: Iodomethane (3.04 g, 21.4 mmol) and NaHCO₃ (1.80 g, 21.4 mmol) were added to a solution of (2R,4S)-1-(((9H-fluoren-9-yl)methoxy)carbonyl)-4-(2-aminoethoxy)pyrrolidine-2-carboxylic acid (1.70 g, 4.3 mmol) in MeOH (15 mL) at RT. The mixture was stirred o.n. at RT. The mixture was filtered, and the filtrate was concentrated under reduced pressure. The residue was purified by Rp-flash column chromatography with the following conditions: Column: Flash C18 (330 g); Mobile Phase A: water (0.1% HCl), Mobile Phase B: MeCN; Gradient Elution: 2-70%; Detector: UV 210 nm. The fractions containing the product were concentrated under reduced pressure to afford 2-(((3S,5R)-1-(((9H-fluoren-9-yl)methoxy) carbonyl)-5-carboxypyrrolidin-3-yl)oxy)-N,N,N-trimethylethan-1-aminium chloride. MS ESI calculated for C₂₅H₃₁N₂O₅ [M]⁺ 439.22, found 439.15. ¹H NMR (400 MHz, methanol-d₄): δ 7.82-7.59 (m, 4H), 7.41-7.26 (m, 4H), 4.40-4.38 (m, 2H), 4.36-4.34 (m, 3H), 4.07-3.90 (m, 2H), 3.79-3.44 (m, 4H), 3.13 (s, 9H), 2.54-2.31 (m, 1H), 2.10-1.92 (m, 1H).

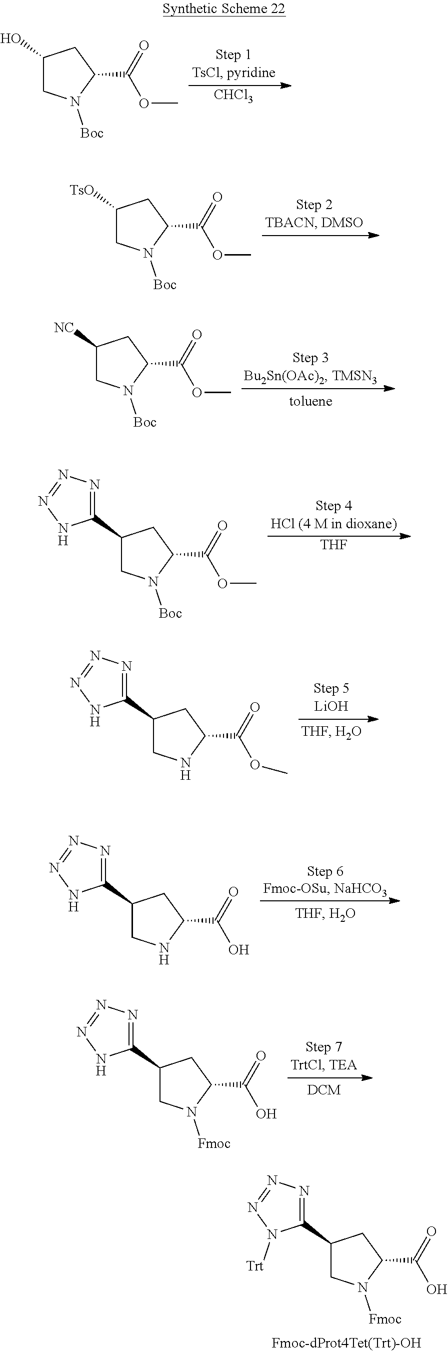

### (2R,4S)-1-(((9H-Fluoren-9-yl)methoxy)carbonyl)-4-(1-trityl-1H-tetrazol-5-yl)pyrrolidine-2-carboxylic acid (Fmoc-dProt4Tet(Trt)-OH)

Step 1: Tosyl chloride (31.1 g, 163 mmol) and pyridine (19.8 mL, 245 mmol) were added to a solution of 1-(tert-butyl) 2-methyl (2R,4R)-4-hydroxypyrrolidine-1,2-dicarboxylate (20.0 g, 82 mmol) in CHCl₃ (200 mL) at RT. The mixture was stirred at RT for 24 h. The mixture was quenched with 0.1 M HCl and extracted with DCM (2×350 mL). The combined organic layers were washed with brine (2×250 mL), dried over anh. Na₂SO₄, filtered, and concentrated under reduced pressure. The residue was purified by silica gel chromatography, eluting with EtOAc in PE to afford 1-(tert-butyl) 2-methyl (2R,4R)-4-(tosyloxy)pyrrolidine-1,2-dicarboxylate. MS ESI calculated for C₁₈H₂₅NO₇SNa [M+Na]⁺ 422.14, found 422.05.

Step 2: A mixture of 1-(tert-butyl) 2-methyl (2R,4R)-4-(tosyloxy)pyrrolidine-1,2-dicarboxylate (15 g, 38 mmol) and tetrabutylammonium cyanide (25 g, 94 mmol) in DMSO (150 mL) was heated o.n. at 45° C. The mixture was quenched with water (100 mL) and extracted with EtOAc (2×300 mL). The combined organic layers were washed with brine (2×200 mL), dried over anh. Na₂SO₄, filtered, and concentrated under reduced pressure. The residue was purified by silica gel chromatography eluting with EtOAc in PE to afford 1-(tert-butyl) 2-methyl (2R,4S)-4-cyanopyrrolidine-1,2-dicarboxylate. MS ESI calculated for C₁₂H₁₈N₂O₄-Boc [M−Boc]⁺ 155.13, found 155.10. ¹H NMR (300 MHz, chloroform-d): δ 4.53-4.35 (m, 1H), 4.01-3.84 (m, 1H), 3.75 (s, 3H), 3.72-3.57 (m, 1H), 3.35-3.17 (m, 1H), 2.63-2.42 (m, 1H), 2.42-2.30 (m, 1H), 1.47-1.42 (m, 9H).

Step 3: A mixture of 1-(tert-butyl) 2-methyl (2R,4S)-4-cyanopyrrolidine-1,2-dicarboxylate (3.5 g, 14 mmol), dibutylstannanediyl diacetate (4.83 g, 13.8 mmol), and trimethylsilyl azide (3.65 mL, 27.5 mmol) in toluene (30 mL) was heated and stirred o.n. at 50° C. The mixture was concentrated under reduced pressure. The residue was purified by Rp-flash column chromatography with the following conditions: Column: Flash C18 (330 g); Mobile Phase A: water (0.05% TFA), Mobile Phase B: MeCN; Gradient Elution: 2-26%; Detector: UV 210 nm. The fractions containing the product were concentrated under reduced pressure to afford 1-(tert-butyl) 2-methyl (2R,4S)-4-(1H-tetrazol-5-yl)pyrrolidine-1,2-dicarboxylate. MS ESI calculated for C₁₂H₁₉N₅O₄Na [M+Na]⁺ 320.14, found 320.00. ¹H NMR (300 MHz, acetonitrile-d₃): δ 4.48-4.38 (m, 1H), 3.99-3.82 (m, 2H), 3.74 (d, J=6.1 Hz, 3H), 3.71-3.58 (m, 1H), 2.75-2.39 (m, 2H), 1.46-1.41 (m, 9H).

Step 4: A solution of HCl (4.0 M in 1,4-dioxane, 30 mL, 120 mmol) was added to a solution of 1-(tert-butyl) 2-methyl (2R,4S)-4-(1H-tetrazol-5-yl)pyrrolidine-1,2-dicarboxylate (2.8 g, 9.4 mmol) in THF (30 mL) at RT. The mixture was stirred at RT for 8 h. The mixture was concentrated under reduced pressure to afford methyl (2R,4S)-4-(1H-tetrazol-5-yl)pyrrolidine-2-carboxylate, which was used in Step 5 without purification. MS ESI calculated for C₇H₁₂N₅O₂ [M+H]⁺ 198.09, found 198.05.

Step 5: An aq. 1 N solution of LiOH (16.5 mL, 16.5 mmol) was added to a solution of methyl (2R,4S)-4-(1H-tetrazol-5-yl)pyrrolidine-2-carboxylate (2.5 g, 8.2 mmol) in THF (17 mL) at RT. The mixture was stirred at RT for 2 h. The mixture was acidified to pH ˜5 with 1 M HCl and then concentrated under reduced pressure to afford (2R,4S)-4-(1H-tetrazol-5-yl)pyrrolidine-2-carboxylic acid, which was used in the Step 6 without purification. MS ESI calculated for C₆H₁₀N₅O₂[M+H]⁺ 184.08, found 184.05.

Step 6: NaHCO₃(2.98 g, 35.5 mmol) and Fmoc-OSu (2.63 g, 7.81 mmol) were added to a solution of (2R,4S)-4-(1H-tetrazol-5-yl)pyrrolidine-2-carboxylic acid (2.6 g, 7.1 mmol) in THF (20 mL) and water (20 mL) at RT. The mixture was stirred at RT for 4 h. The mixture was acidified to pH ˜5 with 1 M HCl and then concentrated under reduced pressure. The residue was purified by Rp-flash column chromatography with the following conditions: Column: Flash C18 (330 g); Mobile Phase A: water (0.05% TFA), Mobile Phase B: MeCN; Gradient Elution: 2-45%; Detector: UV 210 nm. The fractions containing the product were concentrated under reduced pressure to afford (2R,4S)-1-(((9H-fluoren-9-yl)methoxy)carbonyl)-4-(1H-tetrazol-5-yl)pyrrolidine-2-carboxylic acid. MS ESI calculated for C₂₁H₂₀N₅O₄ [M+H]⁺ 406.14, found 406.05.

Step 7: TEA (1.34 mL, 9.62 mmol) and trityl chloride (2.15 g, 7.70 mmol) were added to a solution of (2R,4S)-1-(((9H-fluoren-9-yl)methoxy)carbonyl)-4-(1H-tetrazol-5-yl)pyrrolidine-2-carboxylic acid (2.6 g, 6.4 mmol) in DCM (30 mL) at 0° C. The mixture was stirred at 0° C. for 30 min, and then warmed to RT and stirred for 2 h. The mixture was concentrated under reduced pressure. The residue was purified by Rp-flash column chromatography with the following conditions: Column: Flash C18 (330 g); Mobile Phase A: water, Mobile Phase B: MeCN; Gradient Elution: 2-72%; Detector: UV 210 nm. The fractions containing the product were concentrated under reduced pressure to afford (2R,4S)-1-(((9H-fluoren-9-yl)methoxy)carbonyl)-4-(1-trityl-1H-tetrazol-5-yl)pyrrolidine-2-carboxylic acid. MS ESI calculated for C₄₀H₃₂N₅O₄ [M−H]⁻ 646.25, found 646.20. ¹H NMR (300 MHz, DMSO-d₆): δ 12.95 (s, 1H), 7.97-7.80 (m, 2H), 7.70-7.54 (m, 2H), 7.47-7.14 (m, 13H), 7.07-6.86 (m, 6H), 4.55-4.08 (m, 4H), 3.96-3.79 (m, 2H), 3.78-3.65 (m, 1H), 2.73-2.55 (m, 1H), 2.43-2.24 (m, 1H).

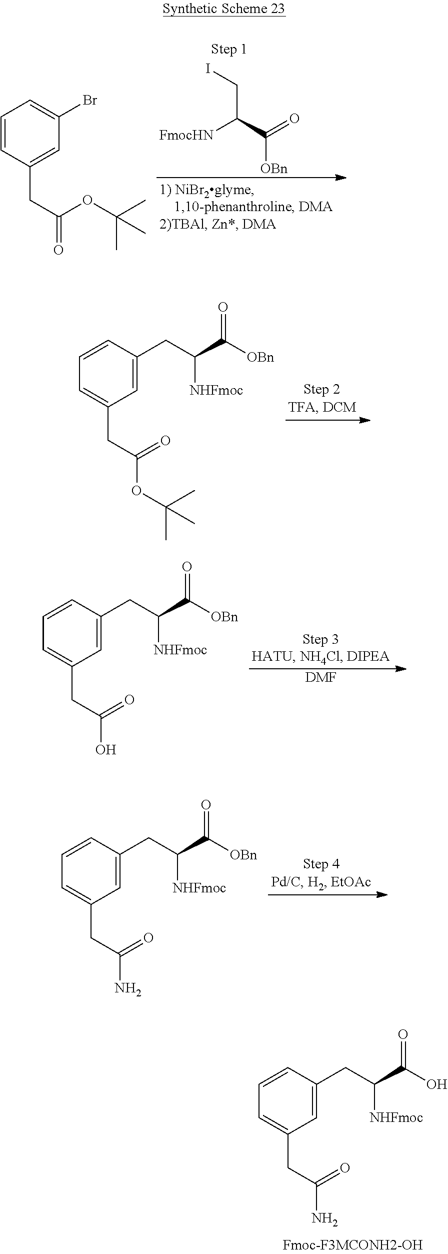

### (S)-2-((((9H-Fluoren-9-yl)methoxy)carbonyl)amino)-3-(3-(2-amino-2-oxoethyl)phenyl)propanoic acid (Fmoc-F3MCONH2-OH)

Step 1: A solution of NiCl₂·glyme (0.81 g, 3.69 mmol) and 1,10-phenanthroline (0.67 g, 3.69 mmol) in anh. DMA (50 mL) was heated at 50° C. for 30 min. The mixture of tert-butyl 2-(3-bromophenyl)acetate (5.00 g, 18.44 mmol), benzyl (R)-2-((((9H-fluoren-9-yl)methoxy) carbonyl)amino)-3-iodopropanoate (9.72 g, 18.44 mmol), and TBAI (6.81 g, 18.44 mmol) in anh. DMA (50 mL) was added at RT. Activated zinc (2.41 g, 36.9 mmol) was added and stirred at RT for 1 h. The reaction mixture was filtrated and concentrated in vacuo. The residue was purified by Rp-flash column chromatography with the following conditions: Column: Flash C18 (330 g); Mobile Phase A: water (0.1% TFA), Mobile Phase B: MeCN; Gradient Elution: 5-82%; Detector: UV 210 nm. The fractions containing the product were concentrated under reduced pressure to afford benzyl (S)-2-((((9H-fluoren-9-yl)methoxy)carbonyl)amino)-3-(3-(2-(tert-butoxy)-2-oxoethyl)phenyl)propanoate. MS ESI calculated for C₃₇H₃₈NO₆ [M+H]⁺ 592.26, found 592.45. ¹H NMR (300 MHz, chloroform-d): δ 7.76 (d, J=7.5 Hz, 2H), 7.56 (d, J=7.5 Hz, 2H), 7.44-7.27 (m, 9H), 7.19-7.09 (m, 2H), 7.05-6.83 (m, 2H), 5.30 (s, 2H), 4.75-4.68 (m, 1H), 4.50-4.31 (m, 2H), 4.23-4.09 (m, 1H), 3.11 (d, J=5.9 Hz, 2H), 2.87-2.61 (m, 2H), 1.42 (s, 9H).

Step 2: TFA (20 mL) was added at RT to a stirred solution of benzyl (S)-2-((((9H-fluoren-9-yl)methoxy)carbonyl)amino)-3-(3-(2-(tert-butoxy)-2-oxoethyl)phenyl)propanoate (4.1 g, 6.9 mmol) in DCM (20 mL). The solution was stirred at RT for 4 h. The solvent was concentrated under reduced pressure to give (S)-2-(3-(2-((((9H-fluoren-9-yl)methoxy)carbonyl) amino)-3-(benzyloxy)-3-oxopropyl)phenyl)acetic acid, which was directly used in Step 3. MS ESI calculated for C₃₃H₃₀NO₆ [M+H]⁺ 536.20, found 536.10.

Step 3: NH₄Cl (0.66 g, 12.32 mmol), HATU (3.51 g, 9.24 mmol), and DIPEA (2.15 mL, 12.32 mmol) at RT were added to a stirred solution of (S)-2-(3-(2-((((9H-fluoren-9-yl)methoxy) carbonyl)amino)-3-(benzyloxy)-3-oxopropyl)phenyl)acetic acid (3.3 g, 6.2 mmol) in DMF (35 mL). The mixture was stirred at RT for 4 h. The reaction mixture was quenched with water (300 mL) and extracted with EtOAc (3×300 mL). The combined organic layers were washed with brine (200 mL), dried over anh. Na₂SO₄ and concentrated under reduced pressure. The residue was purified by silica gel column chromatography, eluting with MeOH in DCM (10:1; v/v) to afford benzyl (S)-2-((((9H-fluoren-9-yl)methoxy)carbonyl)amino)-3-(3-(2-amino-2-oxoethyl) phenyl)propanoate. MS ESI calculated for C₃₃H₃₁N₂O₅ [M+H]⁺ 535.22, found 535.40.

Step 4: Benzyl (S)-2-((((9H-fluoren-9-yl)methoxy)carbonyl) amino)-3-(3-(2-amino-2-oxoethyl)phenyl)propanoate (3.2 g, 6.0 mmol) was dissolved in THF (60 mL). The flask was evacuated and refilled with N₂ (5 times). Pd/C (10% w/w, 0.637 g, 0.60 mmol) was added into the flask. The flask was evacuated and backfilled with H₂ (5 times). The resulting mixture was stirred for 3 h at RT under an atmosphere of dihydrogen. The flask was evacuated and refilled with N₂ (5 times). The resulting mixture was filtered through a Celite® pad. The filtered cake was washed with THF (2×30 mL). The filtrate was concentrated under reduced pressure. The residue was recrystallized from MeCN to give (S)-2-((((9H-fluoren-9-yl)methoxy)carbonyl) amino)-3-(3-(2-amino-2-oxoethyl)phenyl)propanoic acid. MS ESI calculated for C₂₆H₂₅N₂O₅ [M+H]⁺ 445.17, found 445.15. ¹H NMR (300 MHz, methanol-d₄): δ 7.77 (d, J=7.5 Hz, 2H), 7.59 (d, J=7.5 Hz, 2H), 7.44-7.09 (m, 8H), 4.53-4.05 (m, 4H), 3.47 (s, 2H), 3.24-3.17 (m, 1H), 2.98-2.90 (m, 1H).

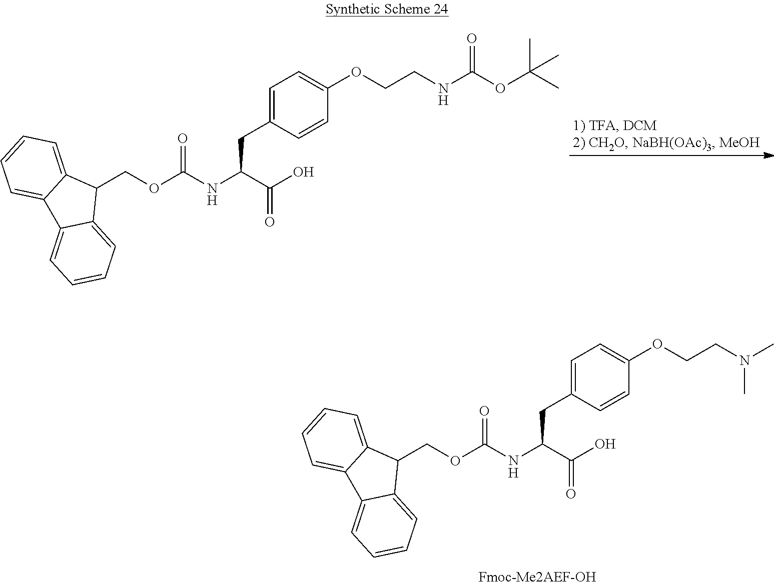 [SMILES: CC(C)(C)OC(=O)NCCOc1ccc(C[C@H](NC(=O)OCC2c3ccccc3-c3ccccc32)C(=O)O)cc1 ; CN(C)CCOc1ccc(C[C@H](NC(=O)OCC2c3ccccc3-c3ccccc32)C(=O)O)cc1]

### (S)-2-((((9H-Fluoren-9-yl)methoxy)carbonyl)amino)-3-(4-(2 (dimethylamino)ethoxy)phenyl)propanoic acid (Fmoc-Me2AEF-OH)

TFA (5 mL) was added to a stirred solution of (S)-2-((((9H-fluoren-9-yl)methoxy) carbonyl)amino)-3-(4-(2-((tert-butoxycarbonyl)amino)ethoxy)phenyl)propanoic acid (1.0 g, 1.8 mmol) in DCM (5 mL) at RT. The solution was stirred at RT for 1 h. The solution was then concentrated under reduced pressure and residual TFA was co-evaporated with toluene (3×15 mL) under vacuum. The crude product was used directly without any further purification.

The crude was taken up in MeOH (20 mL). Formaldehyde (660 μL; 37 wt. % in water; 8.9 mmol) and sodium triacetoxyborohydride (1.89 g, 8.90 mmol) were added at RT. The reaction mixture was stirred o.n. at RT. The reaction mixture was quenched with several drops of 1 N HCl solution and concentrated in vacuo. The residue was purified by Rp-flash column chromatography with the following conditions: Luknova® SuperSep® C18 FR040-1 (55 g, Luknova Inc., Mansfield, MA); Mobile Phase A: water (0.1% TFA), Mobile Phase B: MeCN (0.1% TFA); Gradient Elution: 10-90% over 10 column volumes, flow: 40 mL/min; Detector: UV 215 nm. The product-containing fractions were combined, concentrated under reduced pressure and freeze-dried to afford (S)-2-((((9H-fluoren-9-yl)methoxy)carbonyl)amino)-3-(4-(2 (dimethylamino)ethoxy)phenyl)propanoic acid. MS ESI calculated mass for C₂₈H₃₀N₂O₅ [M+H]⁺ 475.22, found 475.3. ¹H NMR (400 MHz, DMSO-d₆): δ 12.77 (br. s 1H), 9.77 (br. s, 1H), 7.89 (d, J=7.5 Hz, 2H), 7.72 (d, J=8.6 Hz, 1H), 7.66 (d, J=7.0 Hz, 2H), 7.42 (t, J=7.3 Hz, 2H), 7.34-7.28 (m, 2H), 7.23 (d, J=8.1 Hz, 2H), 6.90 (d, J=8.3 Hz, 2H), 4.30-4.21 (m, 2H), 4.21-4.08 (m, 4H), 3.51-3.47 (m, 2H), 3.03 (dd, J=3.7, 13.8 Hz, 1H), 2.84 (s, 6H).

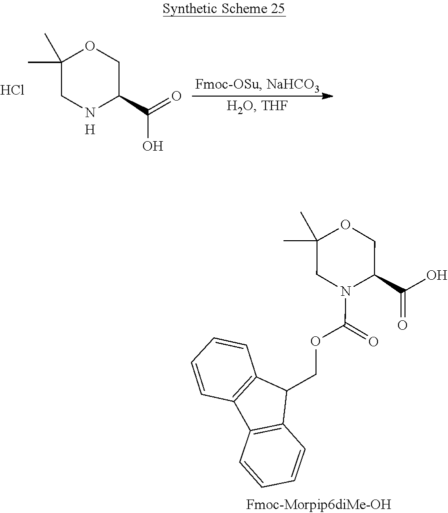 [SMILES: Cl ; CC1(C)CN[C@H](C(=O)O)CO1 ; CC1(C)CN(C(=O)OCC2c3ccccc3-c3ccccc32)[C@H](C(=O)O)CO1]

### (S)-4-(((9H-Fluoren-9-yl)methoxy)carbonyl)-6,6-dimethylmorpholine-3-carboxylic acid (Fmoc-Morpip6diMe-OH)

Fmoc-OSu (1.7 g, 5.1 mmol) at RT then followed by NaHCO₃ (1.8 g, 21 mmol), were added to a stirred solution of (S)-6,6-dimethylmorpholine-3-carboxylic acid hydrochloride (0.82 g, 4.2 mmol) in THF (20 mL) and water (20 mL). The resulting mixture was stirred o.n. at RT. The pH was adjusted to 5 with 1 N HCl and extracted with EtOAc (2×70 mL). The combined organic layers were washed with brine (50 mL), dried over anh. Na₂SO₄ and filtered. The filtrate was concentrated under reduced pressure and the residue was purified by silica gel column chromatography, eluting with MeOH 0-10% in DCM to give (S)-4-(((9H-fluoren-9-yl)methoxy) carbonyl)-6,6-dimethylmorpholine-3-carboxylic acid. MS ESI calculated for C₂₂H₂₃NO₅ [M+H]⁺ 382.16, found 382.2. ¹H NMR (600 MHz, DMSO-d₆): δ 13.08 (s, 1H), 7.90 (t, J=7.3 Hz, 2H), 7.67-7.56 (m, 2H), 7.41 (t, J=7.2 Hz, 2H), 7.35-7.28 (m, 2H), 4.53-4.42 (m, 1H), 4.42-4.35 (m, 1H), 4.35-4.22 (m, 2H), 3.95 (t, J=10.9 Hz, 1H), 3.88-3.79 (m, 1H), 3.36-3.32 (m, 2H), 2.84 (m, 1H), 1.10 (s, 3H), 1.07 (s, 3H). ¹³C NMR (151 MHz, DMSO-d₆): δ 171.1, 155.5, 143.7, 140.8, 127.7, 127.1, 125.2, 125.0, 120.1, 70.4/69.9, 67.0/66.9, 60.4/60.2, 53.4/52.8, 49.7/49.3, 46.7/46.5, 27.3/27.1, 20.4/20.3.

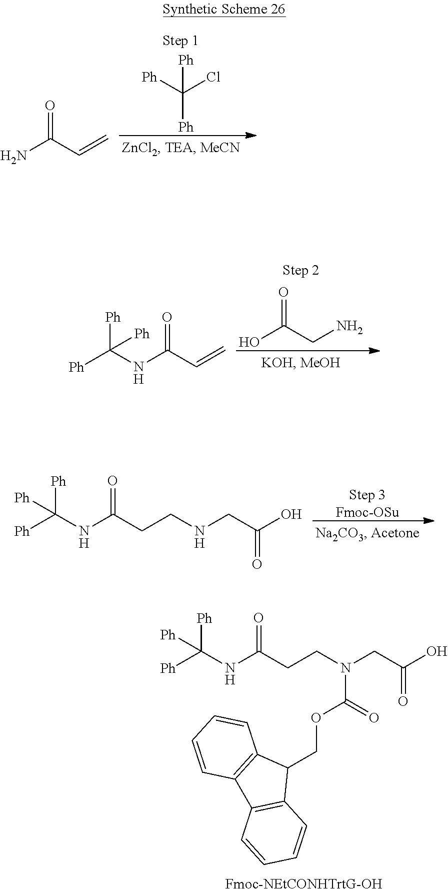

### N-(((9H-Fluoren-9-yl)methoxy)carbonyl)-N-(3-oxo-3-(tritylamino)propyl)glycine (Fmoc-NEtCONHTrtG-OH)

Step 1: Acrylamide (2.00 g, 28.1 mmol) was added to a stirred mixture of triphenylmethyl chloride (7.84 g, 28.1 mmol) and zinc(II) chloride (3.83 g, 28.1 mmol) in MeCN (50 mL). The resulting mixture was stirred at RT for 10 min. Subsequently, a solution of triethylamine (3.92 mL, 28.1 mmol) in MeCN (20 mL) was added to the mixture over 15 min. The reaction was stirred at RT for an additional 15 min. After quenching with an aq. 5% sodium citrate tribasic dihydrate solution and stirring for an additional 15 min, the organic solvent was concentrated in vacuo upon which precipitation occurred. The product was recovered by filtration, triturated with water followed by Et₂O, and dried to yield N-tritylacrylamide. MS ESI calculated for C₂₂H₂₀NO [M+H]⁺ 314.4, found 314.3. ¹H NMR (500 MHz, chloroform-d): δ 7.36-7.22 (m, 15H), 6.71 (s, 1H), 6.41-6.13 (m, 2H), 5.68 (dd, J=9.1, 2.5 Hz, 1H).

Step 2: Glycine (0.766 g, 10.2 mmol) was added to a stirred solution of KOH (0.607 g, 10.8 mmol) in MeOH (55 mL), and the reaction was stirred at RT for 10 min. N-tritylacrylamide (3.20 g, 10.2 mmol) was added to the mixture and the reaction was stirred o.n. at 60° C. The mixture was cooled to RT, acidified with AcOH (1.5 mL), and allowed to stand, upon which precipitation occurred. The residue was filtered and triturated with MeOH followed by DCM to yield (3-oxo-3-(tritylamino)propyl)glycine. MS ESI calculated for C₂₄H₂₅N₂O₃ [M+H]⁺ 389.47, found 389.41. ¹H NMR (500 MHz, methanol-d₄): δ 7.36-7.18 (m, 15H), 3.81 (s, 2H), 3.26 (t, J=6.6 Hz, 2H), 2.87 (t, J=6.6 Hz, 2H).

Step 3: A mixture of N-(9-fluorenylmethoxycarbonyloxy)succinimide (1.28 g, 3.79 mmol), (3-oxo-3-(tritylamino)propyl)glycine (1.34 g, 3.45 mmol) and Na₂CO₃ (0.804 g, 7.59 mmol) in a 1:1 (v/v) mixture of water and acetone (40 mL) was stirred o.n. at RT. The mixture was partially concentrated in vacuo to remove acetone and neutralized to pH 7 by addition of 1 N HCl. The mixture was extracted with EtOAc (2×50 mL), washed with brine (50 mL), dried over anh. Na₂SO₄, filtered, and concentrated in vacuo. The solids were triturated with Et₂O to yield N-(((9H-fluoren-9-yl)methoxy)carbonyl)-N-(3-oxo-3-(tritylamino)propyl)glycine. MS ESI calculated for C₃₉H₃₅N₂O₅ [M+H]⁺ 611.71, found 611.50. ¹H NMR (500 MHz, DMSO-d₆): δ 12.75 (s, 1H), 8.76 (s, 0.5H), 8.64 (s, 0.5H), 7.90 (t, J=8.4 Hz, 2H), 7.68 (d, J=7.5 Hz, 1H), 7.62 (d, J=7.5 Hz, 1H), 7.43 (q, J=7.3 Hz, 2H), 7.34-7.14 (m, 17H), 4.33-4.17 (m, 3H), 3.86 (s, 1H), 3.83 (s, 1H), 3.46 (t, J=6.8 Hz, 1H), 3.38 (t, J=6.8 Hz, 1H), 2.61-2.54 (m, 2H).

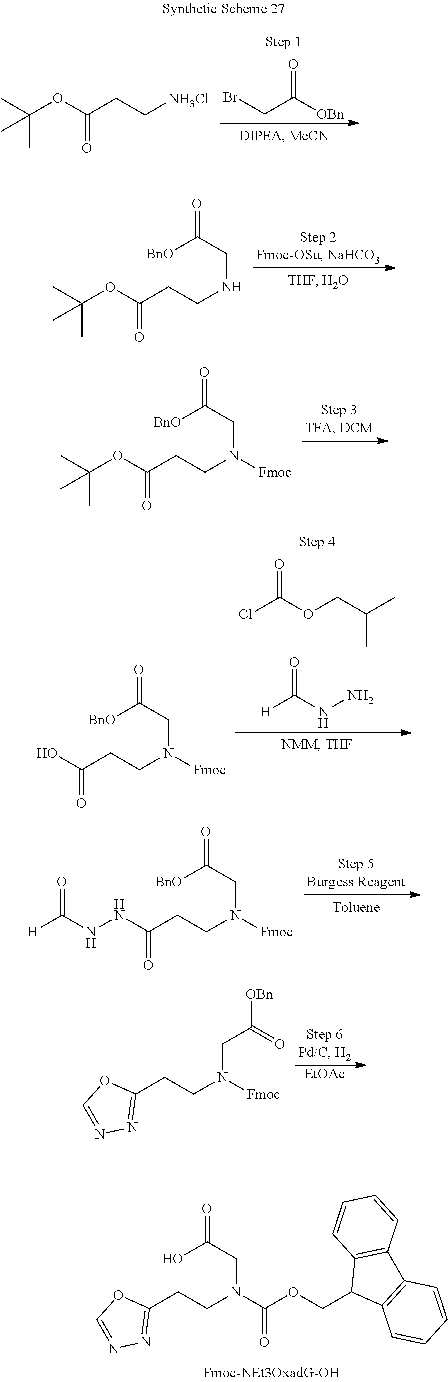

### N-(2-(1,3,4-Oxadiazol-2-yl)ethyl)-N-(((9H-fluoren-9-yl)methoxy)carbonyl)glycine (Fmoc-NEt3OxadG-OH)

Step 1: tert-Butyl 2-bromoacetate (6.0 g, 30.8 mmol) and DIPEA (10.8 mL, 61.5 mmol) at RT were added to a stirred solution of tert-butyl 3-aminopropanoate hydrochloride (16.8 g, 92 mmol) in MeCN (60 mL). The solution was stirred at RT for 4 h. The reaction was concentrated under reduced pressure and purified by silica gel column chromatography, eluting with EtOAc 0-80% in PE to give tert-butyl 3-((2-(benzyloxy)-2-oxoethyl)amino)propanoate. MS ESI calculated for C₁₆H₂₃NO₄Na [M+Na]⁺ 316.16, found 316.25. ¹H NMR (400 MHz, chloroform-d): δ 7.50-7.21 (m, 5H), 5.19 (s, 2H), 3.50 (s, 2H), 2.91-2.88 (m, 2H), 2.51-2.45 (m, 2H), 1.47 (s, 9H).

Step 2: Fmoc-OSu (6.4 g, 19.0 mmol) and NaHCO₃ (8.9 g, 106 mmol) at RT were added to a solution of tert-butyl 3-((2-(benzyloxy)-2-oxoethyl)amino)propanoate (6.2 g, 21.1 mmol) in THF (60 mL) and H₂O (60 mL). The reaction was stirred o.n. at RT. The mixture was extracted with EtOAc (3×250 mL). The combined organic layers were washed with brine (250 mL) and dried over anh. Na₂SO₄. After filtration, the filtrate was concentrated under reduced pressure and the residue was purified by Rp-flash column chromatography with the following conditions: Column: Flash C18; Mobile Phase A: water (0.1% TFA), Mobile Phase B: MeCN; Gradient Elution: 5-70%; Detector: UV 210 nm. The fractions containing the product were concentrated under reduced pressure to afford to give tert-butyl 3-((((9H-fluoren-9-yl)methoxy)carbonyl)(2-(benzyloxy)-2-oxoethyl)amino)propanoate. MS ESI calculated for C₃₁H₃₃NO₆Na [M+Na]⁺ 538.23, found 538.15.

Step 3: TFA (70 mL, 909 mmol) at RT was added to a stirred solution of tert-butyl 3-((((9H-fluoren-9-yl)methoxy)carbonyl)(2-(benzyloxy)-2-oxoethyl)amino)propanoate (7 g, 13.6 mmol) in DCM (70 mL). The solution was stirred at RT for 2 h. The solvent was removed under reduced pressure to give 3-((((9H-fluoren-9-yl)methoxy)carbonyl)(2-(benzyloxy)-2-oxoethyl)amino)propanoic acid, which was used in Step 4 without further purification. MS ESI calculated for C₂₇H₂₆NO₆ [M+H]⁺ 460.17, found 460.30.

Step 4: Isobutyl carbonochloridate (1.6 g, 11.4 mmol) was added to a stirred solution of 3-((((9H-fluoren-9-yl)methoxy)carbonyl)(2-(benzyloxy)-2-oxoethyl)amino)propanoic acid (5.0 g, 10.9 mmol) and 4-methylmorpholine (1.7 g, 16.3 mmol) in THF (50 mL) at −78° C. After 15 min, formic hydrazide (1.0 g, 16.3 mmol) was added, and the resulting mixture was slowly warmed to RT. After 1 h, the reaction mixture was quenched with 0.5 M HCl and extracted with EtOAc (3×250 mL). The combined organic layers were washed with brine (250 mL), dried over anh. Na₂SO₄, filtered, and concentrated under reduced pressure. The residue was purified by silica gel column chromatography, eluting with EtOAc in PE (1:3; v/v) to afford benzyl N-(((9H-fluoren-9-yl)methoxy)carbonyl)-N-(3-(2-formylhydrazineyl)-3-oxopropyl)glycinate. MS ESI calculated for C₂₈H₂₈N₃O₆ [M+H]⁺ 502.19, found 502.35.

Step 5: Methoxycarbonylsulfamoyl)triethylammonium hydroxide (Burgess reagent, 2.5 g, 10.6 mmol) at RT was added to a solution of benzyl N-(((9H-fluoren-9-yl)methoxy)carbonyl)-N-(3-(2-formylhydrazineyl)-3-oxopropyl)glycinate (5.3 g, 10.6 mmol) in toluene (53 mL). The mixture was stirred for 1 h at 110° C. The reaction was cooled to RT and concentrated in vacuum. The residue was purified by silica gel column chromatography, eluting with EtOAc 0-70% in PE to give benzyl N-(2-(1,3,4-oxadiazol-2-yl)ethyl)-N-(((9H-fluoren-9-yl)methoxy) carbonyl)glycinate. MS ESI calculated for C₂₈H₂₆N₃O₅ [M+H]⁺ 484.18, found 484.20.

Step 6: Benzyl N-(2-(1,3,4-oxadiazol-2-yl)ethyl)-N-(((9H-fluoren-9-yl)methoxy) carbonyl)glycinate (3.0 g, 6.2 mmol) was dissolved in EtOAc (60 mL). The flask was evacuated and refilled with N₂ (5 times). Pd/C (0.66 g, 6.2 mmol, dry, 10 wt. %) was added into the flask. The flask was evacuated and backfilled with H₂ (5 times). The resulting mixture was stirred for 2 h at RT under an atmosphere of dihydrogen. The flask was evacuated and refilled with N₂ (5 times). The resulting mixture was filtered through a Celite® pad. The filtered cake was washed with EtOAc (2×30 mL). The filtrate was concentrated under reduced pressure. The residue was recrystallized from Et₂O to afford N-(2-(1,3,4-oxadiazol-2-yl)ethyl)-N-(((9H-fluoren-9-yl) methoxy)carbonyl)glycine. MS ESI calculated for C₂₁H₂₀N₃O₅ [M+H]⁺ 394.13, found 393.95. ¹H NMR (300 MHz, DMSO-d₆): δ 12.71 (s, 1H), 9.14 (d, J=3.8 Hz, 1H), 7.98-7.72 (m, 2H), 7.90-7.80 (m, 2H), 7.46-7.15 (m, 4H), 4.42 (d, J=5.7 Hz, 1H), 4.34-4.15 (m, 2H), 4.04-4.02 (m, 1H), 3.89 (s, 1H), 3.71-3.60 (m, 1H), 3.47-3.42 (m, 1H), 3.17-3.11 (m, 1H), 2.78-2.73 (m, 1H).

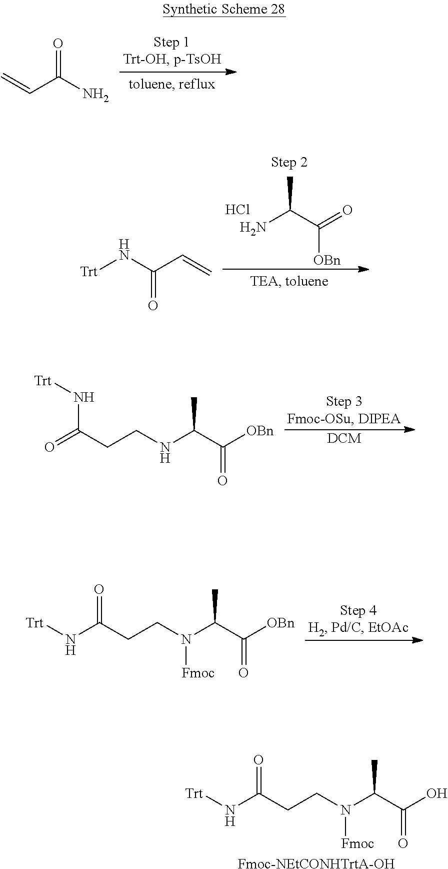

### N-(((9H-Fluoren-9-yl)methoxy)carbonyl)-N-(3-oxo-3-(tritylamino)propyl)-L-alanine (Fmoc-NEtCONHTrt-OH)

Step 1: To a mixture of acrylamide (10.7 g, 150 mmol) in toluene (800 mL) were added triphenylmethanol (58.6 g, 225 mmol) and p-TsOH (7.8 g, 45.0 mmol) at RT. The reaction was stirred and heated o.n. at 110° C. with a Dean-Stark apparatus. The resulting solution was cooled to RT and quenched with aq. sat. NaHCO₃. The aqueous was extracted with EtOAc (3×1000 mL). The combined organic layers were washed with brine (2×200 mL), dried over anh. Na₂SO₄, and filtered. The filtrate was concentrated in vacuum. The residue was purified by silica gel chromatography, eluting with EtOAc 0-35% in PE from to afford N-tritylacrylamide. MS ESI calculated for C₂₂H₂₀NO [M+H]⁺ 314.15, found 314.20. ¹H NMR (300 MHz, chloroform-d): δ 7.37-7.15 (m, 15H), 6.26-6.12 (m, 2H), 5.62-5.60 (m, 1H).

Step 2: TEA (5.6 mL, 40.2 mmol) at RT was added to a suspension of benzyl L-alaninate hydrochloride (8.7 g, 40.2 mmol) in toluene (150 mL). The resulting mixture was stirred at RT for 10 min. N-Tritylacrylamide (8.4 g, 26.8 mmol) was then added to the mixture. The reaction mixture was stirred and heated o.n. at 100° C. The volatiles were concentrated in vacuum. The residue was purified by silica gel chromatography, eluting with EtOAc 0-65% in PE to afford benzyl (3-oxo-3-(tritylamino)propyl)-L-alaninate. MS ESI calculated for C₃₂H₃₃N₂O₃ [M+H]⁺ 493.24, found 493.20. ¹H NMR (300 MHz, chloroform-d): δ 9.03 (s, 1H), 7.42-7.22 (m, 15H), 7.22-7.17 (m, 5H), 5.13 (d, J=1.8 Hz, 2H), 3.44-3.30 (m, 1H), 3.05-2.91 (m, 1H), 2.82-2.69 (m, 1H), 2.42-2.37 (m, 2H), 1.18 (d, J=6.9 Hz, 3H).

Step 3: DIPEA (6.8 mL, 39.0 mmol) and Fmoc-OSu (5.3 g, 15.6 mmol) at RT were added to a solution of benzyl (3-oxo-3-(tritylamino)propyl)-L-alaninate (6.4 g, 13.0 mmol) in DCM (60 mL). The reaction mixture was stirred o.n. at RT and then concentrated under reduced pressure. The residue was purified by silica gel chromatography, eluting with EtOAc 0-45% in PE to afford benzyl N-(((9H-fluoren-9-yl)methoxy)carbonyl)-N-(3-oxo-3-(tritylamino)propyl)-L-alaninate. MS ESI calculated for C₄₇H₄₃N₂O₅ [M+H]⁺ 715.31, found 715.35. ¹H NMR (300 MHz, acetonitrile-d₃): δ 7.89-7.73 (m, 2H), 7.71-7.51 (m, 2H), 7.47-6.94 (m, 24H), 5.18-4.87 (m, 2H), 4.55-4.33 (m, 2H), 4.31-4.12 (m, 2H), 3.52-3.14 (m, 2H), 2.58-2.25 (m, 2H), 2.05-1.97 (m, 3H).

Step 4: Benzyl N-(((9H-fluoren-9-yl)methoxy)carbonyl)-N-(3-oxo-3-(tritylamino)propyl)-L-alaninate (5.3 g, 7.4 mmol) was dissolved in EtOAc (40 mL). The flask was evacuated and refilled with N₂ (5 times). Pd/C (2 g, 18.79 mmol, dry, 10 wt. %) was added into the flask. The flask was evacuated and backfilled with H₂ (5 times). The resulting mixture was stirred for 6 h at RT under an atmosphere of dihydrogen. The flask was evacuated and refilled with N₂ (5 times). The resulting mixture was filtered through a Celite® pad. The filtered cake was washed with EtOAc (2×80 mL). The filtrate was concentrated under reduced pressure. The mixture of diastereomers was separated by SFC: (column: CHIRAL ART® Cellulose-SB (250×30 mm, 5 μm); mobile phase: [0.1% 2 M NH₃-MeOH] in MeOH; B %: 35-35%, 10 min).

The fractions at 6.23 min were combined and concentrated under vacuum to afford N-(((9H-fluoren-9-yl)methoxy)carbonyl)-N-(3-oxo-3-(tritylamino)propyl)-L-alanine. MS ESI calculated for C₄₀H₃₇N₂O₅ [M+H]⁺ 625.26, found 625.20. ¹H NMR (300 MHz, methanol-d₄) δ 7.75 (s, 2H), 7.66-7.57 (m, 2H), 7.42-7.12 (m, 19H), 4.51-4.12 (m, 4H), 3.59-3.34 (m, 2H), 2.70-2.31 (m, 2H), 1.38-1.23 (m, 3H).

The fractions at 7.62 min were combined and concentrated under vacuum to afford N-(((9H-fluoren-9-yl)methoxy)carbonyl)-N-(3-oxo-3-(tritylamino)propyl)-D-alanine. MS ESI calculated for C₄₀H₃₇N₂O₅ [M+H]⁺ 625.26, found 625.15. ¹H NMR (300 MHz, methanol-d₄) δ 7.75 (s, 2H), 7.65-7.57 (m, 2H), 7.42-7.14 (m, 19H), 4.51-4.13 (m, 4H), 3.63-3.34 (m, 2H), 2.71-2.32 (m, 2H), 1.29 (s, 3H).

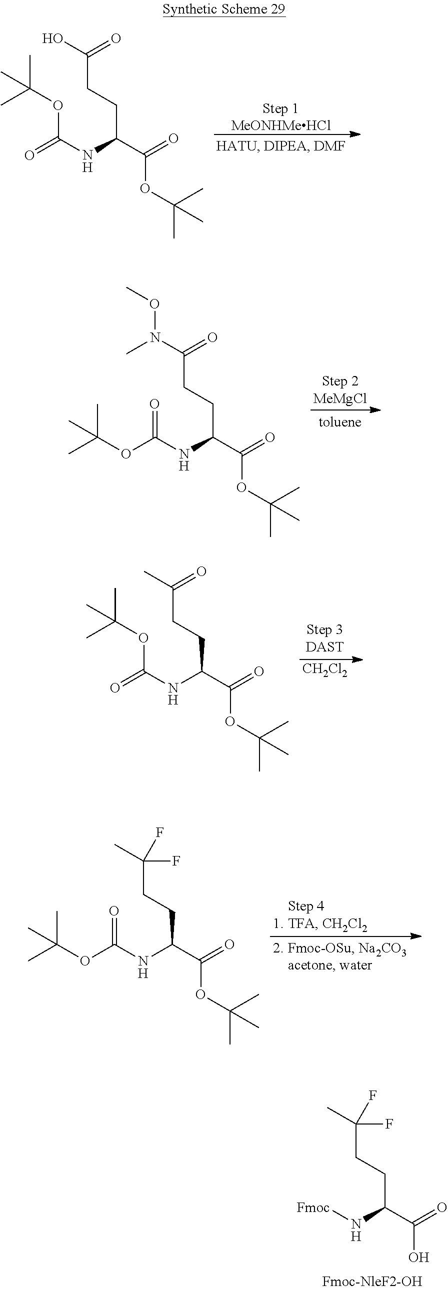

### (S)-2-((((9H-Fluoren-9-yl)methoxy)carbonyl)amino)-5,5-difluorohexanoic acid (Fmoc-NleF2-OH)

Step 1: N,O-dimethylhydroxylamine hydrochloride (2.32 g, 23.7 mmol), HATU (9.02 g, 23.7 mmol), and DIPEA (10.4 mL, 59.3 mmol) were added to a solution of (S)-5-(tert-butoxy)-4-((tert-butoxycarbonyl)amino)-5-oxopentanoic acid (6.00 g, 19.8 mmol) in anh. DMF (25 mL). The resulting solution was stirred at RT for 90 min. The mixture was partitioned between EtOAc (400 mL) and brine (200 mL). The organic phase was washed with brine (2×200 mL), dried over anh. Na₂SO₄, filtered, and concentrated in vacuo. The residue was purified on silica gel column chromatography, eluting with EtOAc 0-50% in hexane to give tert-butyl N²-(tert-butoxycarbonyl)-N⁵-methoxy-N⁵-methyl-L-glutaminate. MS ESI calculated for C₁₆H₃₀N₂O₆ [M+H]⁺ 347.22, found 347.2.

Step 2: A methylmagnesium chloride solution (3.0 M in THF, 33.7 mL, 100 mmol) was added dropwise to a solution of tert-butyl N²-(tert-butoxycarbonyl)-N⁵-methoxy-N⁵-methyl-L-glutaminate (7.0 g, 20 mmol) in anh. THF (30 mL) and toluene (20.0 mL) at −78° C. The resulting solution was stirred at −78° C. for 4 h then allowed to warm up to 0° C. for 5 min. The reaction mixture was then quenched by dropwise addition of an aq. sat. NH₄Cl solution (40 mL). The mixture was partitioned between EtOAc (200 mL), and an aq. sat. NH₄Cl solution (200 mL). The organic phase was washed with aq. sat. NH₄Cl (200 mL), water (2×200 mL), dried over anh. Na₂SO₄, filtered, and concentrated. The residue was purified on silica gel column chromatography, eluting with EtOAc 0-50% in hexane to give tert-butyl (S)-2-((tert-butoxycarbonyl)amino)-5-oxohexanoate. MS ESI calculated for C₁₅H₂₇NO₅ [M+H]⁺ 302.20, found 302.2.

Step 3: (Diethylamino)sulfur trifluoride (6.54 mL, 49.5 mmol) was added to a solution of tert-butyl (S)-2-((tert-butoxycarbonyl)amino)-5-oxohexanoate (5.33 g, 17.7 mmol) in DCM (20 mL) at 0° C. The resulting solution was stirred from 0° C. to RT overnight. The solution was poured dropwise into a mixture of aq. sat. NaHCO₃ solution (300 mL) and DCM (200 mL). The mixture was stirred at RT for 30 min, then extracted with DCM (2×200 mL). The combined organic layers were dried over anh. Na₂SO₄, filtered, and concentrated in vacuo. The residue was purified on silica gel column chromatography, eluting with EtOAc 0-50% in hexane to give tert-butyl (S)-2-((tert-butoxycarbonyl)amino)-5,5-difluorohexanoate. MS ESI calculated for C₁₅H₂₇F₂NO₄ [M+H]⁺ 324.20, found 324.2.

Step 4: TFA (6 mL, 80 mmol) was added to a solution of tert-butyl (S)-2-((tert-butoxycarbonyl)amino)-5,5-difluorohexanoate (1.27 g, 3.93 mmol) in DCM (20 mL). The resulting solution was stirred at RT for 3 h. The volatiles were removed under reduced pressure. The residue was dissolved in a 1:1 (v/v) mixture of acetonitrile and water (20 mL). The residue solution was lyophilized. The resulting solids were dissolved in acetone (10 mL) and water (10 mL). Sodium carbonate (0.83 g, 7.9 mmol) and Fmoc-OSu (1.33 g, 3.93 mmol) were added to the resulting solution. The mixture was stirred at RT for 2 h. The volatiles were partially evaporated under reduced pressure. The aqueous phase was acidified to pH ˜3 by addition of aq. 1 N HCl solution. The mixture was extracted with DCM (3×80 mL). The combined organic layers were dried over anh. Na₂SO₄, filtered, and concentrated in vacuo. The residue was purified on silica gel column chromatography, eluting with MeOH 0-10% in DCM to give (S)-2-((((9H-fluoren-9-yl)methoxy)carbonyl)amino)-5,5-difluorohexanoic acid. MS ESI calculated for C₂₁H₂₁F₂NO₄ [M+H]⁺ 390.15, found 390.3. ¹H NMR (400 MHz, DMSO-d₆): δ 12.61 (s, 1H), 7.90 (d, J=5 Hz, 2H), 7.72 (t, J=7.5 Hz, 2H), 7.42 (t, J=7.5 Hz, 2H), 7.34 (t, J=7.5 Hz, 2H), 4.31-4.30 (m, 2H), 4.25-4.22 (m, 1H), 4.02-3.98 (m, 1H), 1.98-1.84 (m, 4H), 1.61 (t, J=20 Hz, 3H). ¹⁹F NMR (376 MHz, DMSO-d₆): δ −88.6.

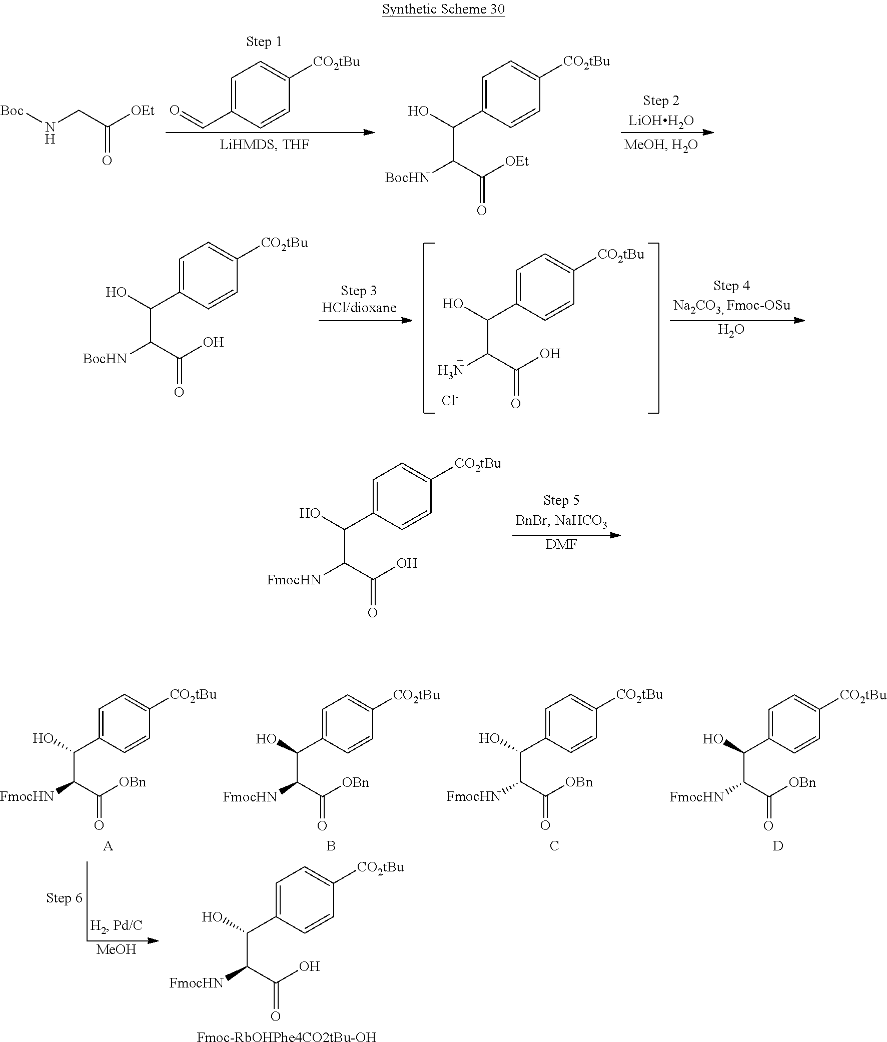

### (2S,3R)-2-((((9H-fluoren-9-yl)methoxy)carbonyl)amino)-3-(4-(tert-butoxycarbonyl)phenyl)-3-hydroxypropanoic acid (Fmoc-RbOHPhe4CO2tBu-OH)

Step 1: Two batches were carried out in parallel. Ethyl (tert-butoxycarbonyl)glycinate (37.0 g, 182 mmol) and anh. THF (1.2 L) were added to a three-neck flask. The flask was purged with N₂ three times, and the solution was cooled to −60° C. Lithium bis(trimethylsilyl)amide solution (2 M in THF, 191 mL) was added dropwise, maintaining the temperature below −60° C. The reaction was stirred at −65° C. for 1 h. tert-Butyl 4-formylbenzoate (30.0 g, 146 mmol) was then added while the mixture was maintained below −60° C. The reaction was then allowed to warm to RT and stirred for 4 h. The two batches were combined and added into aq. sat. NH₄Cl solution (1.0 L) and then extracted by EtOAc (3×500 mL). The combined organic layers were concentrated under reduced pressure to afford the crude product, which was then purified by column chromatography on silica gel eluting with EtOAc in PE (50:1-0:1; v/v) to provide tert-butyl 4-(2-((tert-butoxycarbonyl)amino)-3-ethoxy-1-hydroxy-3-oxopropyl)benzoate as a mixture of four isomers. MS ESI calculated for C₂₁H₃₁NO₇ [M−tBu−Boc+2H]⁺ 254, found 254. ¹H NMR (400 MHz, methanol-d₄): δ 7.92 (d, J=8.0 Hz, 2H), 7.48 (d, J=8.4 Hz, 2H), 5.35-4.90 (m, 1H), 4.50-4.36 (m, 1H), 4.29-4.00 (m, 3H), 1.62-1.55 (m, 9H), 1.32 (m, 9H), 1.28-1.13 (m, 3H).

Step 2: LiOH·H₂O (12.5 g, 298 mmol) was added to a solution of tert-butyl 4-(2-((tert-butoxycarbonyl)amino)-3-ethoxy-1-hydroxy-3-oxopropyl)benzoate (61.0 g, 149 mmol) in DI water (120 mL) and MeOH (400 mL), and the mixture was stirred at RT for 2 h. The mixture was then concentrated under reduced pressure to remove MeOH, and the pH was adjusted to 4-5. The aqueous solution was extracted with EtOAc (2×300 mL), and the combined organic phases were dried over anh. Na₂SO₄, then concentrated under reduced pressure to obtain 2-((tert-butoxycarbonyl)amino)-3-(4-(tert-butoxycarbonyl)phenyl)-3-hydroxypropanoic acid as a mixture of four isomers. MS ESI calculated for C₁₉H₂₇NO₇ [M−Boc+H]⁺ 282, found 282. ¹H NMR (400 MHz, CDCl₃) δ 7.91 (d, J=7.8 Hz, 2H), 7.54-7.39 (m, 2H), 5.44-4.95 (m, 1H), 4.46-4.33 (m, 1H), 1.59 (s, 9H), 1.32 (m, 9H).

Step 3: (Three reactions were carried out in parallel, then combined for the workup and purification in Step 4). 2-((tert-Butoxycarbonyl)amino)-3-(4-(tert-butoxycarbonyl)phenyl)-3-hydroxypropanoic acid (18.2 g, 44.6 mmol) was dissolved in HCl/dioxane (1 M, 119 mL) and stirred at RT for 15 h. Upon completion, the resulting mixture was used directly in the next step.

Step 4: NaHCO₃(14.9 g, 6.93 mL, 178 mmol) was then added to the mixture to adjust the pH to 8 at 0° C., and the aqueous phase was separated (solution 1). A solution of Fmoc-OSu (21.0 g, 62.4 mmol) in dioxane (80 mL) was then added to solution 1, and the mixture was stirred for 2 h at RT. The three batches were then combined and concentrated under reduced pressure to remove dioxane. The pH of the aqueous solution was adjusted to 3-4 using aq. 1 M HCl. The aqueous solution was then extracted with EtOAc (2×200 mL), and the combined organic phases were concentrated under reduced pressure. The residue was purified by silica column chromatography (DCM:MeOH=80:1 to 5:1; v/v) to provide 2-((((9H-fluoren-9-yl)methoxy) carbonyl)amino)-3-(4-(tert-butoxycarbonyl)phenyl)-3-hydroxypropanoic acid as a mixture of four isomers. MS ESI calculated for C₂₉H₂₉NO₇ [M+Na]⁺ 526, found 526. ¹H NMR (400 MHz, methanol-d₄): δ 7.88 (dd, J=8.0, 2.0 Hz, 2H), 7.77 (d, J=7.6 Hz, 2H), 7.52 (t, J=10.4 Hz, 4H), 7.42-7.17 (m, 4H), 5.43-4.55 (m, 1H), 5.00-4.46 (m, 1H), 4.37-4.22 (m, 1H), 4.18-3.92 (m, 3H), 1.51 (m, 9H).

Step 5: NaHCO₃(14.0 g, 167 mmol, 6.49 mL) and BnBr (28.5 g, 19.8 mL, 167 mmol) were added to a solution of 2-((((9H-fluoren-9-yl)methoxy)carbonyl)amino)-3-(4-(tert-butoxycarbonyl)phenyl)-3-hydroxypropanoic acid (28.0 g, 55.6 mmol) in DMF (210 mL). The resulting mixture was stirred o.n. at RT. Water (200 mL) was added to quench the reaction and the aqueous phase was extracted with EtOAc (3×200 mL). The combined organic phases were washed with water (200 mL), dried over anh. Na₂SO₄, filtered, and concentrated in vacuo. The residue was purified by silica gel column chromatography, eluting with EtOAc in PE (80:1-10:1; v/v) to afford tert-butyl 4-(2-((((9H-fluoren-9-yl)methoxy)carbonyl)amino)-3-(benzyloxy)-1-hydroxy-3-oxopropyl)benzoate as a mixture of four isomers. C₃₆H₃₅NO₇ [M+Na]⁺ 616, found 616. The mixture of isomers was then separated by SFC (column: DAICEL CHIRALPAK AD (250×50 mm, 10 μm); mobile phase: [0.1% NH₄OH in EtOH]; [0.1% NH₄OH in EtOH]%: 60-60%, 11 min), then SFC (column: DAICEL CHIRALPAK AD (250×50 mm, 10 μm); mobile phase: [0.1% NH₄OH in EtOH]; [0.1% NH₄OH in EtOH]%: 50-50%, 6.5 min), and then SFC (column: DAICEL CHIRALCEL OD (250×50 mm, 10 μm); mobile phase: [0.1% NH₄OH in EtOH]; [0.1% NH₄OH in EtOH]%: 60-60%, 10 min) to provide compounds A, B, C, and D.

Step 6: Pd/C (529 mg, 2.25 mmol, 45 wt. %) was added to a stirred solution of Compound D (6.00 g, 10.1 mmol) in MeOH (500 mL). The resulting mixture was purged with dihydrogen and stirred under dihydrogen atmosphere o.n. at RT. The suspension was filtered through a pad of Celite®, and the filtrate was concentrated under reduced pressure. The residue was purified by prep-HPLC (column: Phenomenex Luna C18, 80×40 mm, 3 μm; mobile phase: [water(HCl) and MeCN]; MeCN %: 45-75%, 7 min) to provide (2S,3R)-2-((((9H-fluoren-9-yl)methoxy)carbonyl)amino)-3-(4-(tert-butoxycarbonyl)phenyl)-3-hydroxypropanoic acid. C₉H₂₉NO₇ [M−tBu+2H]⁺ 448, found 448. ¹H NMR (400 MHz, methanol-d₄): δ 7.88 (d, J=8.4 Hz, 2H), 7.77 (d, J=7.6 Hz, 2H) 7.57-7.47 (m, 4H), 7.55-7.22 (m, 4H), 5.42 (d, J=7.8 Hz, 1H), 4.46 (d, J=7.6 Hz, 1H), 4.31 (dd, J=9.8, 6.0 Hz, 1H), 4.16-3.97 (m, 2H), 1.52 (s, 9H).

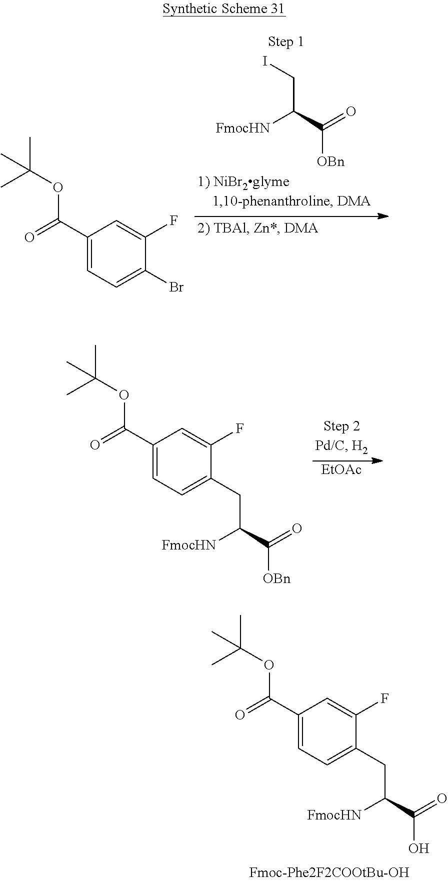

### (S)-2-((((9H-Fluoren-9-yl)methoxy)carbonyl)amino)-3-(4-(tert-butoxycarbonyl)-2-fluorophenyl)propanoic acid (Fmoc-Phe2F4COOtBu-OH)

Step 1: A mixture of NiCl₂·glyme (0.182 g, 0.827 mmol) and 1,10-phenanthroline (0.149 g, 0.827 mmol) in anh. DMA (15 mL) was heated at 50° C. for 1 h. A mixture of tert-butyl 4-bromo-3-fluorobenzoate (1.365 g, 4.96 mmol), benzyl (R)-2-((((9H-fluoren-9-yl)methoxy) carbonyl)amino)-3-iodopropanoate (2.18 g, 4.13 mmol) and TBAI (1.527 g, 4.13 mmol) in anh. DMA (20 mL) was then added at RT. Activated zinc (0.541 g, 8.27 mmol) was added to the solution and the resulting suspension stirred at 40° C. for 4 h. The reaction mixture was quenched with DI water (80 mL) and extracted with EtOAc (2×250 mL). The combined organic layers were washed with brine (3×100 mL), dried over anh. Na₂SO₄, filtered, and concentrated under vacuum. The residue was purified by Rp-flash chromatography with the following conditions: Column: flash C18 (330 g); Mobile Phase A: water (0.1% TFA), Mobile Phase B: MeCN; (Gradient: 5% B hold 5 min, up to 52% B within 33 min, 56% B hold 5.2 min; up to 95% B within 2 min, 95% B hold 10 min); Flow rate: 90 mL/min; Detector: UV 210 nm. The product-containing fractions were collected and concentrated in vacuo to give tert-butyl (S)-4-(2-((((9H-fluoren-9-yl)methoxy) carbonyl)amino)-3-(benzyloxy)-3-oxopropyl)-3-fluorobenzoate. MS ESI calculated for C₃₆H₃₅FNO₆ [M+H]⁺ 596.24, found 596.20. ¹H NMR (300 MHz, DMSO-d₆): δ 8.03-8.01 (m, 1H), 7.89-7.87 (m, 2H), 7.68-7.48 (m, 4H), 7.45-7.31 (m, 3H), 7.31-7.23 (m, 7H), 5.13 (s, 2H), 4.55-4.35 (m, 1H), 4.26-4.24 (m, 2H), 4.14-4.11 (m, 1H), 3.27-3.24 (m, 1H), 3.08-3.00 (m, 1H), 1.53 (s, 9H). ¹⁹F NMR (282 MHz, DMSO-d₆): δ −117.39.

Step 2: Pd/C (0.372 g, 3.5 mmol, 10 wt. %) was added to a stirred solution of tert-butyl (S)-4-(2-((((9H-fluoren-9-yl)methoxy)carbonyl)amino)-3-(benzyloxy)-3-oxopropyl)-3-fluorobenzoate (3.2 g, 5.4 mmol) in EtOAc (60 mL). The resulting mixture was purged with dihydrogen and stirred under dihydrogen atmosphere at RT for 1 h. The suspension was filtered through a pad of Celite®, and the filtrate was concentrated under reduced pressure. The residue was purified by Rp-flash chromatography with the following conditions: Column: C18 (330 g); Mobile Phase A: water (0.05% TFA), Mobile Phase B: MeCN; (Gradient: 5% B hold 5 min, up to 75% B within 30 min, 80% B hold 7 min; up to 95% B within 2 min, 95% B hold 10 min); Flow rate: 100 mL/min; Detector: UV 210 nm. The product-containing fractions were collected and concentrated in vacuo to give (S)-2-((((9H-fluoren-9-yl)methoxy)carbonyl)amino)-3-(4-(tert-butoxycarbonyl)-2-fluorophenyl)propanoic. MS ESI calculated for C₂₉H₂₉FNO₆ [M+NH₃+H]⁺ 523.19, found 523.15. ¹H NMR (300 MHz, DMSO-d₆): δ 12.91 (s, 1H), 7.88-7.80 (m, 3H), 7.69-7.52 (m, 4H), 7.52-7.35 (m, 3H), 7.35-7.22 (m, 2H), 4.33-4.07 (m, 4H), 3.37-3.20 (m, 1H), 2.95-2.91 (m, 1H), 1.51 (s, 9H). ¹⁹F NMR (282 MHz, DMSO-d₆): δ −117.38.

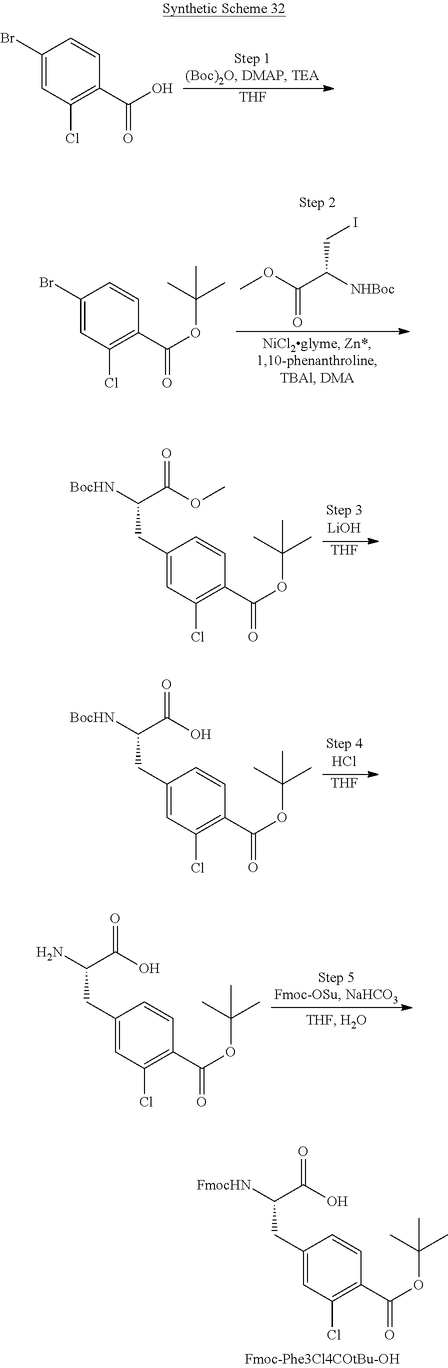

### (S)-2-((((9H-Fluoren-9-yl)methoxy)carbonyl)amino)-3-(4-(tert-butoxycarbonyl)-3-chlorophenyl)propanoic acid (Fmoc-Phe3Cl4COOtBu-OH)

Step 1: DMAP (3.9 g, 31.9 mmol), TEA (22.2 mL, 159 mmol) and di-tert-butyl dicarbonate (27.8 g, 127 mmol) were added to a solution of 4-bromo-2-chlorobenzoic acid (25.0 g, 106 mmol) in THF (250 mL) under Ar at RT. The solution was stirred o.n. at RT. The reaction mixture was concentrated under reduced pressure and the residue was purified by silica gel chromatography, eluting with EtOAc 0-60% in PE to afford tert-butyl 4-bromo-2-chlorobenzoate. ¹H NMR (400 MHz, chloroform-d): δ 7.63-7.60 (m, 2H), 7.44-7.41 (m, 1H), 1.60 (s, 9H).

Step 2: A mixture of nickel (II) chloride ethylene glycol dimethyl ether complex (1.8 g, 8.2 mmol) and 1,10-phenanthroline (7.4 g, 41.2 mmol) in anh. DMA (120 mL) was heated at 50° C. for 30 min. A solution of tert-butyl 4-bromo-2-chlorobenzoate (12.0 g, 41.2 mmol), methyl (R)-2-((tert-butoxycarbonyl) amino)-3-iodopropanoate (14.9 g, 45.3 mmol) and tetrabutylammonium iodide (15.2 g, 41.2 mmol) in anh. DMA (120 mL) was added at RT to the mixture. Activated zinc (5.4 g, 82 mmol) was subsequently added. The mixture was stirred for 24 h at 30° C. The mixture was filtrated and concentrated under reduced pressure. The residue was purified by silica gel chromatography, eluting with EtOAc 0-100% in PE, followed by purification by Rp-flash column chromatography with the following conditions: Column: Flash C18 (330 g); Mobile Phase A: water (5 mM NH₄HCO₃), Mobile Phase B: MeCN; Gradient Elution: 0-75%; Detector: UV 210 nm. The fractions containing the product were concentrated under reduced pressure to afford tert-butyl (S)-4-(2-((tert-butoxycarbonyl) amino)-3-methoxy-3-oxopropyl)-2-chlorobenzoate. MS ESI calculated for C₂₀H₂₈ClNO₆Na [M+Na]⁺ 436.16, found 436.15. ¹H NMR (400 MHz, chloroform-d): δ 7.69-7.67 (m, 1H), 7.19 (s, 1H), 7.07-7.04 (m, 1H), 4.59-4.57 (m, 1H), 3.74 (s, 3H), 3.17-3.13 (m, 1H), 3.04-2.98 (m, 1H), 1.60 (s, 9H), 1.43 (s, 9H).

Step 3: An aq. 1 N solution of LiOH (39 mL, 39 mmol) was added to a solution of tert-butyl (S)-4-(2-((tert-butoxycarbonyl) amino)-3-methoxy-3-oxopropyl)-2-chlorobenzoate (8.00 g, 19.3 mmol) in THF (80 mL) at RT. The mixture was stirred at RT for 2 h. The mixture was concentrated under reduced pressure to afford (S)-3-(4-(tert-butoxycarbonyl)-3-chlorophenyl)-2-((tert-butoxycarbonyl)amino)propanoic acid, which was used in Step 4 without purification. MS ESI calculated for C₁₉H₂₆ClNO₆Na [M+Na]⁺ 422.14, found 422.10.

Step 4: A solution of hydrogen chloride in 1,4-dioxane (4.0 M, 80 mL, 320 mmol) was added to a solution of (S)-3-(4-(tert-butoxycarbonyl)-3-chlorophenyl)-2-((tert-butoxycarbonyl) amino)propanoic acid (8.0 g, 20.0 mmol) in anh. THF (80 mL) under Ar at RT. The mixture was stirred at RT for 1 h. The mixture was concentrated under reduced pressure to afford (S)-2-amino-3-(4-(tert-butoxycarbonyl)-3-chlorophenyl) propanoic acid, which was used in Step 5 without purification. MS ESI calculated for C₁₄H₁₉C₁NO₄ [M+H]⁺ 300.09, found 300.15.

Step 5: Sodium bicarbonate (7.0 g, 83 mmol) and N-(9-fluorenylmethoxycarbonyloxy) succinimide (5.06 g, 15.0 mmol) were added to a solution of (S)-2-amino-3-(4-(tert-butoxycarbonyl)-3-chlorophenyl)propanoic acid (5.0 g, 16.7 mmol) in THF (50 mL) and water (50 mL) under Ar at RT. The mixture was stirred at RT for 1 h. The pH was adjusted to ˜4 with aq. 1 N HCl. The mixture was extracted with EtOAc (2×200 mL). The combined organic layers were washed with brine (150 mL), dried over anh. Na₂SO₄, filtered, and concentrated under reduced pressure. The filtrate was concentrated under reduced pressure and the residue was purified by Rp-flash column chromatography with the following conditions: Column: Flash C18 (330 g); Mobile Phase A: water (0.1% TFA), Mobile Phase B: MeCN; Gradient Elution: 2-60%; Detector: UV 210 nm. The fractions containing the product were concentrated under reduced pressure to afford (S)-2-((((9H-fluoren-9-yl)methoxy)carbonyl)amino)-3-(4-(tert-butoxycarbonyl)-3-chlorophenyl)propanoic acid. MS ESI calculated for C₂₉H₂₇ClNO₆ [M−H]⁺ 520.16, found 520.25. ¹H NMR (400 MHz, methanol-d₄): δ 7.78-7.76 (m, 2H), 7.63-7.49 (m, 3H), 7.38-7.35 (m, 3H), 7.30-7.20 (m, 3H), 4.47-4.43 (m, 1H), 4.37-4.33 (m, 1H), 4.23-4.10 (m, 2H), 3.28-3.23 (m, 1H), 2.98-2.92 (m, 1H), 1.55 (s, 9H).

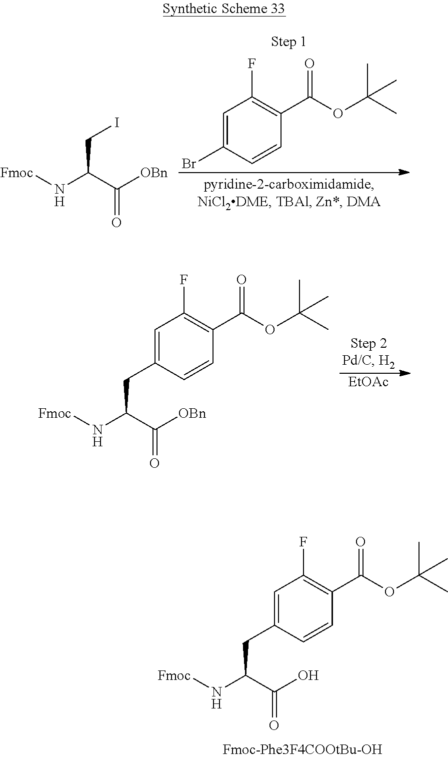

### (S)-2-((((9H-Fluoren-9-yl)methoxy)carbonyl)amino)-3-(4-(tert-butoxycarbonyl)-3-fluorophenyl)propanoic acid (Fmoc-Phe3F4COOtBu-OH)

Step 1: A mixture of nickel (II) chloride ethylene glycol dimethyl ether complex (0.333 g, 1.517 mmol) and pyridine-2-carboximidamide hydrochloride (0.478 g, 3.03 mmol) in anh. DMA (80 mL) was stirred and heated at 50° C. for 1 h. The mixture was cooled to RT. To this solution, a mixture of benzyl (R)-2-((((9H-fluoren-9-yl)methoxy)carbonyl)amino)-3-iodopropanoate (8.00 g, 15.2 mmol), tert-butyl 4-bromo-2-fluorobenzoate (8.35 g, 30.3 mmol), and TBAI (11.2 g, 30.3 mmol) in anh. DMA (80 mL) was added. Subsequently, activated zinc (1.98 g, 30.3 mmol) was added. The mixture was stirred for 2 h at RT. The mixture was diluted with water (200 mL) and extracted with EtOAc (3×200 mL). The combined organic layers were washed with brine (3×100 mL), dried over anh. Na₂SO₄, and filtered. The filtrate was concentrated under reduced pressure. The residue was purified by silica gel chromatography, eluting with EtOAc in PE to afford tert-butyl (S)-4-(2-((((9H-fluoren-9-yl)methoxy)carbonyl)amino)-3-(benzyloxy)-3-oxopropyl)-2-fluorobenzoate. MS ESI calculated for C₃₆H₃₄FNO₆Na [M+Na]⁺ 618.24, found 618.25.

Step 2: tert-Butyl (S)-4-(2-((((9H-fluoren-9-yl)methoxy)carbonyl)amino)-3-(benzyloxy)-3-oxopropyl)-2-fluorobenzoate (4.00 g, 6.72 mmol) was dissolved in EtOAc (50 mL). The flask was evacuated and refilled with N₂ (5 times). Pd/C (0.143 g, 1.34 mmol, dry, 10 wt. %) was added into the flask. The flask was evacuated and backfilled with H₂ (5 times). The resulting mixture was stirred for 4 h at RT under an atmosphere of dihydrogen. The flask was evacuated and refilled with N₂ (5 times). The resulting mixture was filtered through a Celite® pad. The filtered cake was washed with EtOAc (3×100 mL). The filtrate was concentrated under reduced pressure. The residue was purified by Rp-flash column chromatography with the following conditions: Column: Flash C18 (330 g); Mobile Phase A: water, Mobile Phase B: MeCN; Gradient Elution: 0-50%; Detector: UV 210 nm. The fractions containing the product were concentrated under reduced pressure to afford (S)-2-((((9H-fluoren-9-yl)methoxy)carbonyl) amino)-3-(4-(tert-butoxycarbonyl)-3-fluorophenyl)propanoic acid. MS ESI calculated for C₂₉H₂₈FNO₆Na [M+Na]⁺ 528.19, found 528.15. ¹H NMR (300 MHz, DMSO-d₆): δ 12.85 (s, 1H), 7.88 (d, J=7.5 Hz, 2H), 7.77-7.70 (m, 2H), 7.67-7.58 (m, 2H), 7.43-7.37 (m, 2H), 7.32-7.18 (m, 4H), 4.28-4.13 (m, 4H), 3.20-3.14 (m, 1H), 2.96-2.88 (m, 1H), 1.51 (s, 9H).

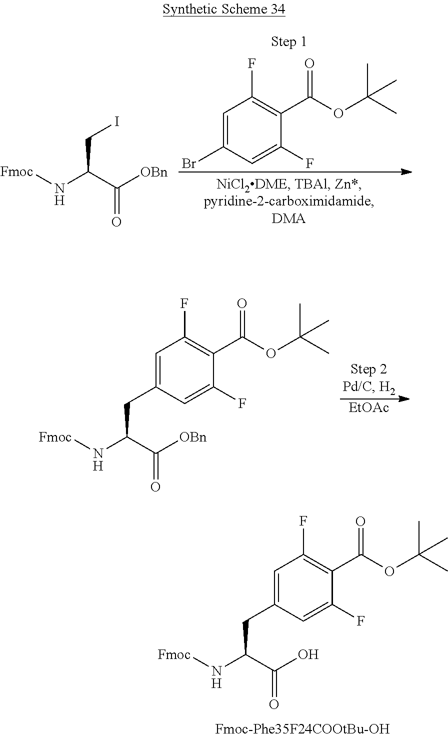

### (S)-2-((((9H-Fluoren-9-yl)methoxy)carbonyl)amino)-3-(4-(tert-butoxycarbonyl)-3,5-difluorophenyl)propanoic acid (Fmoc-Phe35F24COOtBu-OH)

Step 1: A solution of nickel (II) chloride ethylene glycol dimethyl ether complex (0.33 g, 1.52 mmol) and pyridine-2-carboximidamide hydrochloride (0.48 g, 3.03 mmol) in anh. DMA (80 mL) was stirred and heated at 50° C. for 1 h. The mixture was cooled to RT, and a solution of benzyl (R)-2-((((9H-fluoren-9-yl)methoxy)carbonyl)amino)-3-iodopropanoate (8.00 g, 15.2 mmol), tert-butyl 4-bromo-2,6-difluorobenzoate (8.89 g, 30.3 mmol), and TBAI (11.2 g, 30.3 mmol) in anh. DMA (80 mL) was added. Then activated zinc (1.98 g, 30.3 mmol) was added to the mixture. The mixture was stirred for 2 h at RT. The mixture was diluted with water (200 mL) and extracted with EtOAc (3×200 mL). The combined organic layers were washed with brine (3×100 mL), dried over anh. Na₂SO₄, filtered, and concentrated under reduced pressure. The residue was purified by silica gel chromatography, eluting with EtOAc in PE to afford tert-butyl (S)-4-(2-((((9H-fluoren-9-yl)methoxy)carbonyl)amino)-3-(benzyloxy)-3-oxopropyl)-2,6-difluorobenzoate. MS ESI calculated for C₃₆H₃₃F₂NO₆Na [M+Na]⁺ 636.23, found 636.20.

Step 2: tert-Butyl (S)-4-(2-((((9H-fluoren-9-yl)methoxy)carbonyl)amino)-3-(benzyloxy)-3-oxopropyl)-2,6-difluorobenzoate (4.00 g, 6.52 mmol) was dissolved in EtOAc (40 mL). The flask was evacuated and refilled with N₂ (5 times). Pd/C (0.277 g, 2.61 mmol, dry, 10 wt. %) was added into the flask. The flask was evacuated and backfilled with H₂ (5 times). The resulting mixture was stirred for 4 h at RT under an atmosphere of dihydrogen. The flask was evacuated and refilled with N₂ (5 times). The resulting mixture was filtered through a Celite® pad. The filtered cake was washed with EtOAc (3×80 mL). The filtrate was concentrated under reduced pressure. The residue was purified by Rp-flash column chromatography with the following conditions: Column: Flash C18 (330 g); Mobile Phase A: water, Mobile Phase B: MeCN; Gradient Elution: 60-60%; Detector: UV 210 nm. The fractions containing the product were concentrated under reduced pressure to afford (S)-2-((((9H-fluoren-9-yl)methoxy)carbonyl) amino)-3-(4-(tert-butoxycarbonyl)-3,5-difluorophenyl)propanoic acid. MS ESI calculated for C₂₉H₂₇F₂NO₆Na [M+Na]⁺ 546.18, found 546.05. ¹H NMR (300 MHz, DMSO-d₆): δ 12.87 (s, 1H), 7.88 (d, J=7.5 Hz, 2H), 7.77 (d, J=8.4 Hz, 1H), 7.63-7.59 (m, 2H), 7.43-7.38 (m, 2H), 7.32-7.25 (m, 2H), 7.15 (d, J=9.0 Hz, 2H), 4.30-4.17 (m, 4H), 3.19-3.13 (m, 1H), 2.96-2.87 (m, 1H), 1.51 (s, 9H).

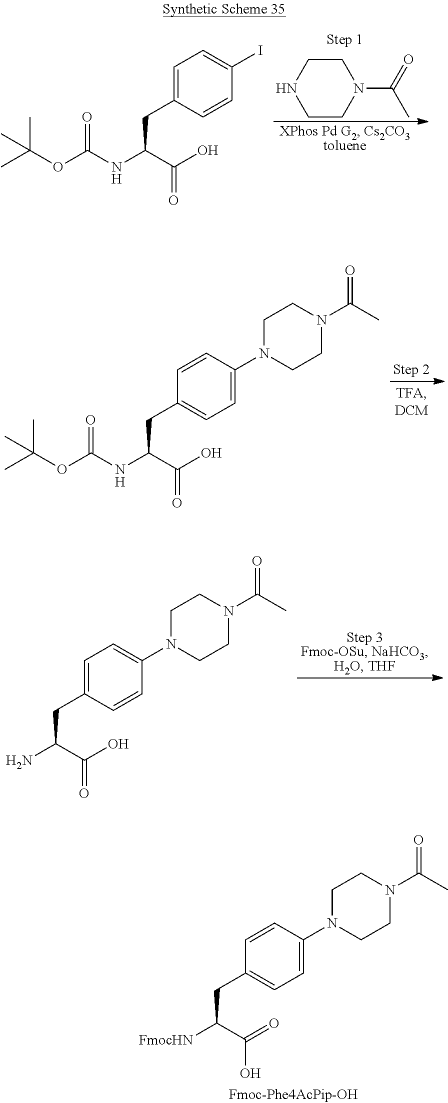

### (S)-2-((((9H-Fluoren-9-yl)methoxy)carbonyl)amino)-3-(4-(4-acetylpiperazin-1-yl)phenyl)propanoic acid (Fmoc-Phe4AcPip-OH)

Step 1: XPhos Pd G₂ (2.06 g, 2.6 mmol) at RT under N₂ was added to a stirred solution of (S)-3-(4-bromophenyl)-2-((tert-butoxycarbonyl)amino) propanoic acid (6.00 g, 17.4 mmol) in toluene (180 mL). The resulting solution was stirred at 100° C. for 10 min. 1-(Piperazin-1-yl) ethan-1-one (2.24 g, 17.4 mmol) and Cs₂CO₃ (5.04 g, 26.1 mmol) were added and the resulting suspension was stirred at 110° C. for 2 h. The reaction was cooled to RT and quenched with DI water (500 mL), extracted with EtOAc (2×500 mL). The combined organic layers were washed with brine (3×200 mL), dried over anh. Na₂SO₄, and filtered. The filtrate was concentrated under reduced pressure and the residue was purified by silica gel column chromatography, eluting with EtOAc 0-60% in PE to give (S)-3-(4-(4-acetylpiperazin-1-yl)phenyl)-2-((tert-butoxycarbonyl)amino)propanoic acid. MS ESI calculated for C₂₀H₃₀N₃O₅[M+H]⁺ 392.21, found 392.25. ¹H NMR (400 MHz, methanol-d₄): δ 7.12 (d, J=8 Hz, 2H), 6.89 (d, J=8 Hz, 2H), 4.16-4.13 (m, 1H), 3.72-3.65 (m, 4H), 3.14-3.04 (m, 4H), 2.93-2.81 (m, 2H), 2.13 (s, 3H), 1.38-1.29 (m, 9H).

Step 2: TFA (30 mL) at RT was added to a stirred solution of (S)-3-(4-(4-acetylpiperazin-1-yl)phenyl)-2-((tert-butoxycarbonyl)amino)propanoic acid (10.0 g, 25.5 mmol) in DCM (30 mL). The solution was stirred at RT for 1 h then concentrated under reduced pressure. The crude product was used directly in the next step without further purification. MS ESI calculated for C₁₅H₂₂N₃O₃[M+H]⁺ 292.16, found 292.20.

Step 3: Fmoc-OSu (6.34 g, 18.8 mmol) and NaHCO₃ (8.77 g, 104 mmol) were added to a stirred solution of (S)-3-(4-(4-acetylpiperazin-1-yl)phenyl)-2-(carboxyamino)propanoic acid (7.00 g, 20.9 mmol) in a 1:1 (v/v) mixture of THF and DI water (50 mL). The resulting mixture was stirred at RT for 2 h. The pH was adjusted to 5 with aq. 1 N HCl solution and extracted with EtOAc (2×200 mL). The combined organic layers were washed with brine (3×100 mL), dried over anh. Na₂SO₄ and filtered. The filtrate was concentrated under reduced pressure and the residue was purified by Rp-flash chromatography with the following conditions: Column: Flash C18 (330 g); Mobile Phase A: water (0.1% TFA), Mobile Phase B: MeCN; (Gradient: 5% B hold 5 min, up to 55% B within 15 min, 55% B hold 5 min; up to 95% B within 20 min, 95% B hold 5 min); Flow rate: 90 mL/min; Detector: UV 210 nm. The product-containing fractions were combined and concentrated in vacuo to give (S)-2-((((9H-fluoren-9-yl)methoxy)carbonyl) amino)-3-(4-(4-acetylpiperazin-1-yl)phenyl)propanoic acid. MS ESI calculated for C₃₀H₃₂N₃O₅ [M+H]⁺ 514.23, found 514.30. ¹H NMR (400 MHz, methanol-d₄): δ 7.78-7.76 (m, 2H), 7.61-7.52 (m, 2H), 7.40-7.37 (m, 2H), 7.32-7.22 (m, 4H), 7.08-6.98 (m, 2H), 4.47-4.43 (m, 1H), 4.33-4.31 (m, 1H), 4.14-4.02 (m, 2H), 3.68-3.63 (m, 4H), 3.23-3.08 (m, 5H), 2.91-2.85 (m, 1H), 2.11 (s, 3H).

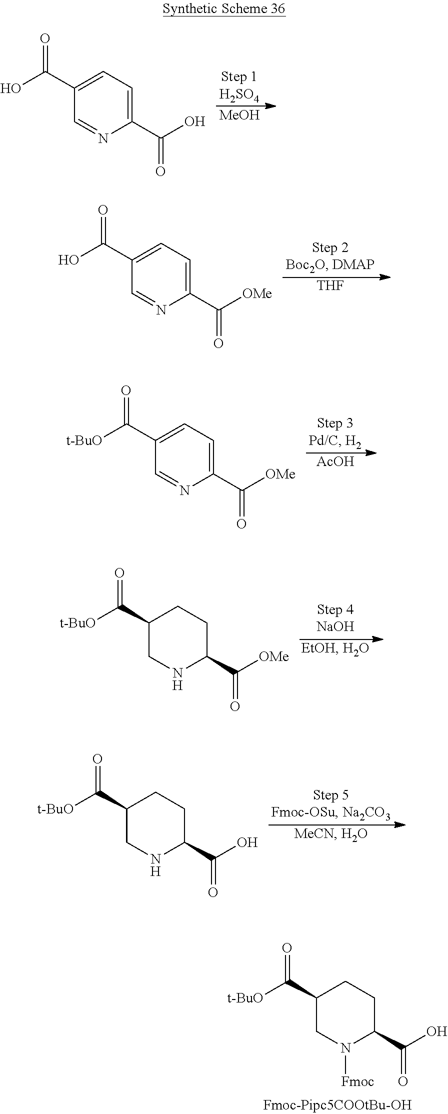

### (2S,5S)-1-(((9H-Fluoren-9-yl)methoxy)carbonyl)-5-(tert-butoxycarbonyl)piperidine-2-carboxylic acid (Fmoc-Pipc5COOtBu-OH)

Step 1: H₂SO₄(37.6 g, 365 mmol) was added to a stirred solution of pyridine-2,5-dicarboxylic acid (100 g, 598 mmol) in MeOH (700 mL) at RT. The solution was heated to 60° C. for 7 h. The crude reaction mixture was poured into water (1 L) at 15° C. The resulting precipitate was collected by filtration. The filtered solids were then washed with MeOH to give 6-(methoxycarbonyl)nicotinic acid. ¹H NMR (400 MHz, DMSO-d₆): δ 9.16 (dd, J=1.6, 7.6 Hz, 1H), 8.42-8.47 (m, 1H), 8.16 (t, J=8.0 Hz, 1H), 3.91 (s, 3H).

Step 2: 6-(Methoxycarbonyl)nicotinic acid (40.0 g, 220 mmol) was dissolved in MeOH (280 mL) at RT. DMAP (13.4 g, 110 mmol) and Boc₂O (120 g, 126 mL, 552 mmol) were added and the reaction mixture was stirred at RT for 12 h. The solvent was removed under reduced pressure, and the solids were partitioned between brine (300 mL) and EtOAc (300 mL). The organic layer was washed with brine (100 mL), dried over anh. Na₂SO₄, filtered, and concentrated under vacuum. The residue was purified by silica gel column chromatography, eluting with EtOAc in PE (20:1-5:1; v/v) to give 5-(tert-butyl) 2-methyl pyridine-2,5-dicarboxylate. ¹H NMR (400 MHz, DMSO-d₆): δ 9.24 (dd, J=1.6 Hz, 1H), 8.37 (dd, J=2.0, 8.0 Hz, 1H), 8.18 (d, J=8.0 Hz, 1H), 4.03 (s, 3H), 1.62 (s, 9H).

Step 3: 5-(tert-Butyl) 2-methyl pyridine-2,5-dicarboxylate (30.0 g, 126 mmol) was added to a suspension of Pd/C (3.00 g, 17 mmol, 10 wt. %) in AcOH (600 mL) at RT. The reaction mixture was purged with dihydrogen (three times) then heated at 50° C., with stirring for 12 h under H₂ (50 Psi). The solids were filtered through a Celite® pad, rinsed with AcOH. The filtrate was concentrated under reduced pressure. The solids were partitioned between EtOAc (300 mL) and brine (300 mL). The organic layer was washed with brine, dried over anh. Na₂SO₄, filtered, and concentrated under vacuum to afford 5-(tert-butyl) 2-methyl (2S,5S)-piperidine-2,5-dicarboxylate. ¹H NMR (400 MHz, chloroform-d): δ 3.71 (s, 3H), 3.47 (m, 1H), 3.13-3.12 (m, 1H), 2.88 (dd, J=3.6, 12.8 Hz, 1H), 2.36 (m, 3H), 2.03-1.99 (m, 3H), 1.85-1.80 (m, 5H), 1.43 (s, 9H).

Step 4: A solution of NaOH (4.93 g, 123 mmol) in H₂O (100 mL) was added to a stirred solution of 5-(tert-butyl) 2-methyl (2S,5S)-piperidine-2,5-dicarboxylate (20.0 g, 82.2 mmol) in EtOH (120 mL) at RT. The reaction mixture was stirred at RT for 12 h. The organic volatiles were removed under reduced pressure to afford (2S,5S)-5-(tert-butoxycarbonyl)piperidine-2-carboxylic acid. ¹H NMR (400 MHz, DMSO-d₆): δ 14.08 (s, 1H), 8.54 (d, J=8.8 Hz, 1H), 7.48 (s, 1H), 7.29 (d, J=8.8 Hz, 1H), 3.55 (s, 3H).

Step 5: Fmoc-OSu (26.4 g, 78.5 mmol) and Na₂CO₃ (99.8 g, 94.2 mmol) were added to a stirred solution of (2S,5S)-5-(tert-butoxycarbonyl)piperidine-2-carboxylic acid (18.0 g, 78.5 mmol) in DI water (50 mL) and MeCN (75 mL) at RT. The solution was stirred at RT for 12 h. The organic volatiles were removed under reduced pressure and the solids were partitioned between EtOAc (150 mL) and brine (150 mL). The organic layer was washed with brine, dried over anh. Na₂SO₄, filtered, and concentrated under vacuum. The residue was purified by prep-HPLC (column: Phenomenex™ Luna™ C18 (250×70 mm, 15 μm, (Phenomenex™, Torrance, CA)); mobile phase: [water(HCl)-MeCN]; B %: 50-80%, 20 min). The compound was further purified by SFC (column: Daicel ChiralPak® IG (250×50 mm, 10 μm, Daicel Chiral Technologies, West Chester, PA); mobile phase: [0.1% NH₄OH in water-EtOH]; B %: 45-45%, 6 min) to provide (2S,5S)-1-(((9H-fluoren-9-yl)methoxy)carbonyl)-5-(tert-butoxycarbonyl) piperidine-2-carboxylic acid. ¹H NMR (400 MHz, DMSO-d₆): δ 7.88-7.86 (m, 2H), 7.67-7.62 (m, 2H), 7.41-7.32 (m, 4H), 4.59 (s, 1H), 4.37-4.23 (m, 3H), 4.11-3.94 (dd, J=12.4, 5.4 Hz, 1H), 3.02-2.89 (m, 1H), 2.27-2.16 (m, 2H), 1.85-1.82 (m, 1H), 1.55-1.53 (m, 2H), 1.40 (m, 9H), 1.28-1.22 (m, 2H).

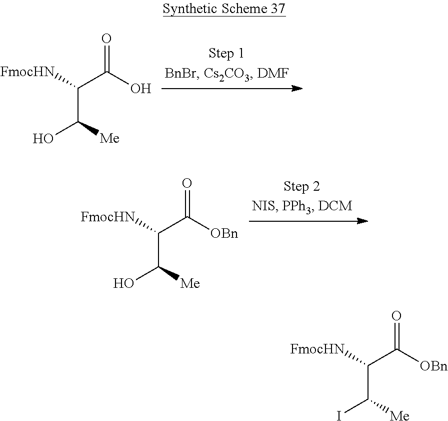

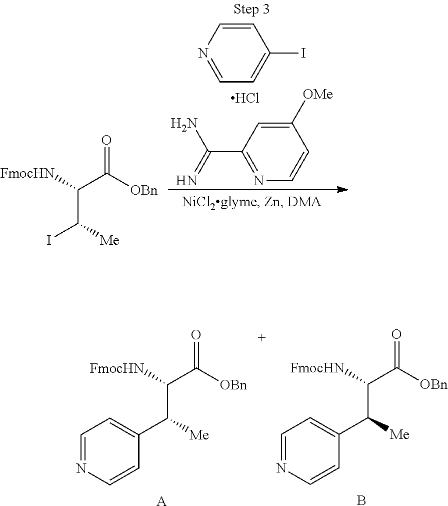

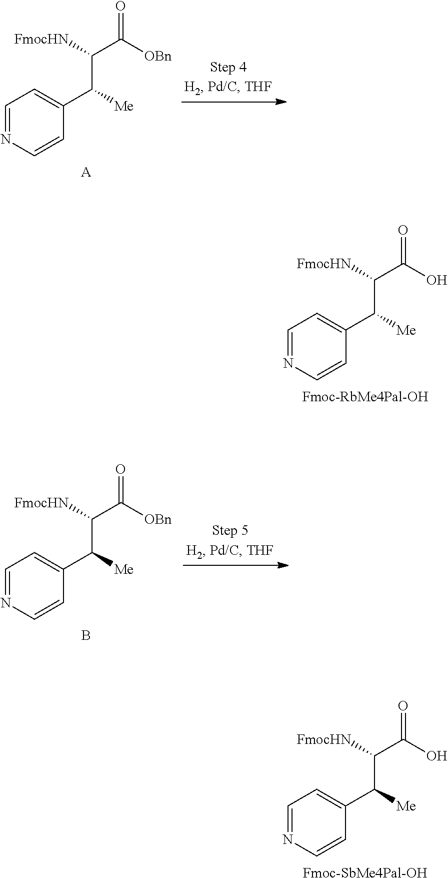

### (2S,3R)-2-((((9H-Fluoren-9-yl)methoxy)carbonyl)amino)-3-(pyridin-4-yl)butanoic acid (Fmoc-RbMe4Pal-OH1) and (2S,3S)-2-((((9H-fluoren-9-yl)methoxy)carbonyl)amino)-3-(pyridin-4-yl)butanoic acid (Fmoc-SbMe4Pal-OH)

Step 1: BnBr (501 g, 2.93 mol) at RT dropwise was added to a solution of (((9H-fluoren-9-yl)methoxy)carbonyl)-L-threonine (500 g, 1.46 mol) in DMF (3.00 L). Cs₂CO₃ (954 g, 2.93 mol) was added into the solution. The reaction mixture was stirred at RT for 3 h. The reaction mixture was poured into DI water (20 L) at RT, and solid precipitated. The solids were recovered by filtration and the filter cake was rinsed with DI water (2×1 L). The filtered cake was dried under vacuum. The crude product was triturated with MTBE (10 L) at RT for 2 h. The solids were recovered by filtration and the filtered cake was dried under reduced pressure. The crude product was triturated with PE (10 L) at RT for 6 h. The solids were recovered by filtration and the filtered cake was dried under reduced pressure to give benzyl (((9H-fluoren-9-yl)methoxy) carbonyl)-L-threoninate. ¹H NMR (400 MHz, methanol-d₄): δ 7.79 (d, J=7.6 Hz, 2H), 7.66-7.56 (m, 2H), 7.40-7.27 (m, 10H), 5.19 (s, 2H), 4.42-4.19 (m, 5H), 1.18 (d, J=6.4 Hz, 3H).

Step 2: NIS (547 g, 2.43 mol) was added at 0° C. to a solution of benzyl (((9H-fluoren-9-yl)methoxy)carbonyl)-L-threoninate (350 g, 811 mmol) in DCM (3.5 L). PPh₃ (638 g, 2.43 mol) was added to the stirred solution. The reaction mixture was stirred at 30° C. for 5 h. The reaction mixture was concentrated under reduced pressure and the residue was purified by silica gel column chromatography (PE/DCM/EtOAc=10:10:1; v/v/v) to give benzyl (2R,3S)-2-((((9H-fluoren-9-yl)methoxy)carbonyl)amino)-3-iodobutanoate. ¹H NMR (400 MHz, methanol-d₄): δ 7.79 (d, J=7.6 Hz, 2H), 7.68 (d, J=7.2 Hz, 2H), 7.40-7.27 (m, 10H), 5.20 (q, J=9.6 Hz, 2H), 4.53-4.50 (m, 2H), 4.42-4.37 (m, 2H), 4.24-4.21 (m, 1H), 1.84-1.79 (m, 3H).

Step 3: Solution A: A solution of NiCl₂·glyme (4.06 g, 18.5 mmol) and 4-methoxypicolinimidamide hydrochloride (3.47 g, 18.5 mmol) in anh. DMA (500 mL) was stirred at RT for 30 min under N₂. Solution B: A solution of benzyl (2R,3S)-2-((((9H-fluoren-9-yl)methoxy)carbonyl)amino)-3-iodobutanoate (100 g, 185 mmol) and 4-iodopyridine (37.9 g, 185 mmol) in anh. DMA (250 mL) was stirred at RT for 30 min under N₂. Solution B was slowly added to Solution A at RT under N₂. Activated zinc (60.4 g, 924 mmol) was added in portions to the solution. The suspension was stirred at RT o.n. under N₂. The solids were removed by filtration and the filtered cake was rinsed with EtOAc (3×800 mL). The filtrate was poured into DI water (2.0 L). The suspension was extracted with EtOAc (3×1.5 L). The combined organic layers were washed with brine (2×800 mL), dried over anh. Na₂SO₄, filtered, and concentrated under reduced pressure. The residue was purified by prep-HPLC (column: Phenomenex™ Luna™ C18 250 mm, 10 μm, (Phenomenex™, Torrance, CA); mobile phase: [water (0.225% FA)-MeCN]; MeCN %: 40-70%, 20 min) to give benzyl (2S,3R)-2-((((9H-fluoren-9-yl)methoxy)carbonyl)amino)-3-(pyridin-4-yl)butanoate and benzyl (2S,3S)-2-((((9H-fluoren-9-yl)methoxy)carbonyl)amino)-3-(pyridin-4-yl)butanoate as a mixture. Part of the mixture was separated by SFC chiral separation (column: Daicel ChiralPak® AD (250×50 mm, 10 μm, Daicel Chiral Technologies, West Chester, PA); mobile phase: [0.1% NH₄OH in IPA]; IPA %: 50-50%, 6.2 min). The two separated products were concentrated under vacuum to give both diastereoisomers.

Benzyl (2S,3R)-2-((((9H-fluoren-9-yl)methoxy)carbonyl)amino)-3-(pyridin-4-yl)butanoate A: ¹H NMR (400 MHz, DMSO-d₆): δ 8.44 (d, J=5.6 Hz, 2H), 7.95 (d, J=8.0 Hz, 1H), 7.87-7.55 (m, 3H), 7.56-7.54 (m, 2H), 7.38-7.32 (m, 9H), 5.17 (d, J=2.8 Hz, 2H), 4.37 (t, J=9.2 Hz, 1H), 4.18-4.08 (m, 3H), 3.20-3.15 (m, 1H), 1.21-1.14 (m, 3H).

Benzyl (2S,3S)-2-((((9H-fluoren-9-yl)methoxy)carbonyl)amino)-3-(pyridin-4-yl)butanoate B: ¹H NMR (400 MHz, DMSO-d₆): δ 8.43 (d, J=5.6 Hz, 2H), 8.11 (d, J=8.0 Hz, 1H), 7.89 (d, J=7.6 Hz, 2H), 7.68 (d, J=7.2 Hz, 2H), 7.44-7.41 (m, 2H), 7.32-7.26 (m, 6H), 7.24-7.16 (m, 2H), 4.96 (dd, J=35.6, 12.8 Hz, 2H), 4.41 (t, J=8.4 Hz, 1H), 4.26-4.16 (m, 3H), 3.28-3.23 (m, 1H), 1.25 (d, J=6.8 Hz, 3H).

Step 4: Pd/C (5.00 g, 10 wt. %) was added to a solution of benzyl (2S,3R)-2-((((9H-fluoren-9-yl)methoxy)carbonyl)amino)-3-(pyridin-4-yl)butanoate A (52.0 g, 106 mmol) in THF (300 mL) under N₂. The flask was evacuated and backfilled with H₂ (3 times). The resulting mixture was stirred for 12 h at RT under an atmosphere of dihydrogen (15 psi). The flask was evacuated and refilled with N₂ (5 times). The resulting mixture was filtered, and the filtered cake was washed with THF (10×200 mL). The filtrate was concentrated under reduced pressure. The crude product was triturated with MTBE (100 mL) at RT for 30 min. The suspension was filtered and the filtered cake was dried under vacuum to give (2S,3R)-2-((((9H-fluoren-9-yl) methoxy)carbonyl)amino)-3-(pyridin-4-yl)butanoic acid. MS ESI calculated for C₂₄H₂₂N₂O₄ [M+H]⁺ 403, found 403. ¹H NMR (400 MHz, T=273+80K, DMSO-d₆): δ 8.44 (d, J=4.8 Hz, 2H), 7.85 (d, J=7.6 Hz, 2H), 7.58 (d, J=6.4 Hz, 2H), 7.38 (d, J=7.6 Hz, 2H), 7.32-7.26 (m, 4H), 4.29-4.10 (m, 4H), 3.21-3.15 (m, 1H), 1.26 (d, J=6.8 Hz, 3H).

Step 5: Pd/C (4.00 g, 10 wt. %) was added to a solution of benzyl (2S,3S)-2-((((9H-fluoren-9-yl)methoxy)carbonyl)amino)-3-(pyridin-4-yl)butanoate B (75.0 g, 152 mmol) in THF (380 mL) under N₂. The flask was evacuated and backfilled with H₂ (3 times). The resulting mixture was stirred for 12 h at RT under an atmosphere of dihydrogen (15 psi). The flask was evacuated and refilled with N₂ (5 times). The resulting mixture was filtered, and the filtered cake was washed with THF (10×200 mL). The filtrate was concentrated under reduced pressure. The crude product was triturated with MTBE (100 mL) at RT for 30 min. The suspension was filtered and the filtered cake was dried under vacuum to give (2S,3S)-2-((((9H-fluoren-9-yl) methoxy)carbonyl)amino)-3-(pyridin-4-yl)butanoic acid. MS ESI calculated for C₂₄H₂₂N₂O₄ [M+H]⁺ 403, found 403. ¹H NMR (400 MHz, T=273+80K, DMSO-d₆): δ 8.44 (d, J=4.8 Hz, 2H), 7.86 (d, J=7.6 Hz, 2H), 7.68-7.62 (m, 2H), 7.41 (d, J=7.6 Hz, 2H), 7.32-7.25 (m, 4H), 4.33-4.25 (m, 3H), 4.19 (q, J=6.8 Hz, 1H), 3.30-3.28 (m, 1H), 1.27 (d, J=6.4 Hz, 3H).

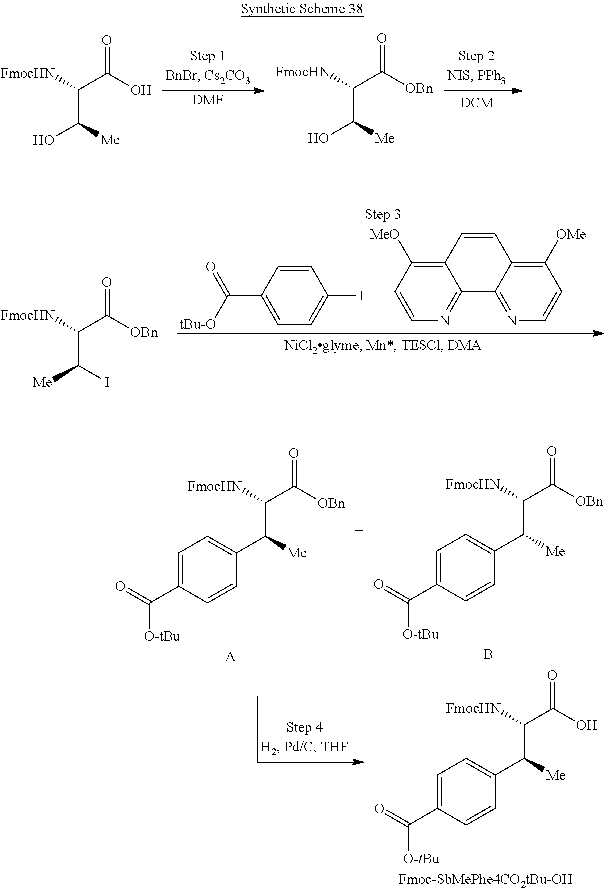

### (2S,3S)-2-((((9H-Fluoren-9-yl)methoxy)carbonyl)amino)-3-(4-(tert-butoxycarbonyl)phenyl)butanoic acid (Fmoc-SbMePhe4CO2tBu-OH)

Step 1: To a solution of (((9H-fluoren-9-yl)methoxy)carbonyl)-L-threonine (500 g, 1.46 mol) in DMF (3.00 L) was added BnBr (501 g, 2.93 mol) dropwise at RT. Cs₂CO₃ (954 g, 2.93 mol) was added into the solution and the resulting suspension was stirred at RT for 3 h. The reaction mixture was poured into DI water (20 L) at RT. The solids were recovered by filtration. The filtered cake was rinsed with DI water (2×1 L) and dried under vacuum to give the crude product. The crude solids were triturated with MTBE (10 L) at RT for 2 h. The suspension was filtered, and the resulting filtered cake dried under reduced pressure. The residue was triturated with PE (10 L) at RT for 6 h. The suspension was filtered, and the solids dried under reduced pressure to give benzyl (((9H-fluoren-9-yl)methoxy)carbonyl)-L-threoninate. ¹H NMR (400 MHz, methanol-d₄): δ 7.79 (d, J=7.6 Hz, 2H), 7.66-7.65 (m, 2H), 7.40-7.27 (m, 10H), 5.19 (s, 2H), 4.42-4.19 (m, 5H), 1.18 (d, J=6.4 Hz, 3H).

Step 2: To a solution of benzyl (((9H-fluoren-9-yl)methoxy)carbonyl)-L-threoninate (350 g, 811 mmol) in DCM (3.5 L) were added N-iodosuccinimide (547 g, 2.43 mol) and PPh₃ (638 g, 2.43 mol) at 0° C. The solution was stirred at 30° C. for 5 h. The reaction mixture was concentrated under reduced pressure. The resulting residue was purified by silica gel column chromatography (PE/DCM/EtOAc=10:10:1; v/v/v) to give benzyl (2R,3S)-2-((((9H-fluoren-9-yl)methoxy)carbonyl)amino)-3-iodobutanoate. ¹H NMR (400 MHz, methanol-d₄): δ 7.79 (d, J=7.6 Hz, 2H), 7.68 (d, J=7.2 Hz, 2H), 7.40-7.27 (m, 10H), 5.20 (q, J=9.6 Hz, 2H), 4.53-4.50 (m, 2H), 4.42-4.37 (m, 2H), 4.24-4.21 (m, 1H), 1.84-1.79 (m, 3H).

Step 3: (Carried out three reactions in parallel, then combined for workup and purification) In a nitrogen-filled glovebox, NiCl₂·glyme (3.25 g, 14.8 mmol) and 4,7-dimethoxy-1,10-phenanthroline (3.55 g, 14.8 mmol) were added into anh. DMA (500 mL). The mixture was stirred at RT for 30 min. Then benzyl (2R,3S)-2-((((9H-fluoren-9-yl)methoxy)carbonyl)amino)-3-iodobutanoate (80.0 g, 148 mmol) and tert-butyl 4-iodobenzoate (44.9 g, 148 mmol) were added to the solution. Then manganese (16.2 g, 296 mmol) was added followed by TMSCl (8.03 g, 9.38 mL, 73.9 mmol). The suspension was stirred at RT for 12 h under N₂. Then the three reaction suspensions were combined and filtered, and the filter cake was rinsed with EtOAc (2×500 mL). The filtrate was poured into water (2.0 L). The organic layer and the aqueous phase were separated, and the aqueous phase was extracted with EtOAc (3×800 mL). The combined organic phase was washed with brine (2×1 L) and dried over anh. sodium sulfate. The organic phase was concentrated under vacuum to afford a residue, which was purified first by Rp-HPLC (Phenomenex Titan C18 Bulk 250×100 mm, 10 μm; mobile phase: [aqueous 10 mM NH₄HCO₃ and MeCN]; MeCN %: 75-100%, 20 min) to give the product as a mixture of two diastereomers. The isomers were separated by SFC chiral separation (DAICEL CHIRALPAK AD (250×50 mm, 10 μm); mobile phase: [EtOH]; EtOH %: 40-40%, 3.6 min) to provide the two separated diastereomers.

tert-Butyl 4-((2S,3S)-3-((((9H-fluoren-9-yl)methoxy)carbonyl)amino)-4-(benzyloxy)-4-oxobutan-2-yl)benzoate (A). ¹H NMR (400 MHz, chloroform-d): δ 7.85 (d, J=8 Hz, 2H), 7.78 (d, J=7.6 Hz, 2H), 7.54 (t, J=7.2 Hz, 2H), 7.43-7.28 (m, 8H), 7.05 (d, J=8 Hz, 2H), 5.19-5.03 (m, 3H), 4.70-4.66 (m, 1H), 4.49-4.44 (m, 1H), 4.37-4.33 (m, 1H), 4.22-4.18 (m, 1H), 3.45-3.42 (m, 1H), 1.60 (s, 9H), 1.32 (d, J=7.2 Hz, 3H).

tert-Butyl 4-((2R,3S)-3-((((9H-fluoren-9-yl)methoxy)carbonyl)amino)-4-(benzyloxy)-4-oxobutan-2-yl)benzoate (B). ¹H NMR (400 MHz, chloroform-d): δ 7.79 (d, J=8 Hz, 2H), 7.72 (d, J=7.6 Hz, 2H), 7.52 (d, J=7.6 Hz, 2H), 7.38-7.25 (m, 7H), 7.11-6.93 (m, 3H), 5.27 (d, J=9.2 Hz, 1H), 4.97 (q, J=8.4 Hz, 2H), 4.57 (t, J=7.6 Hz, 1H), 4.41-4.27 (m, 2H), 4.14 (t, J=6.8 Hz, 1H), 3.22-3.16 (m, 1H), 1.54 (s, 9H), 1.28 (d, J=7.2 Hz, 3H).

Step 4: To a solution of tert-butyl 4-((2S,3S)-3-((((9H-fluoren-9-yl)methoxy)carbonyl) amino)-4-(benzyloxy)-4-oxobutan-2-yl)benzoate A (34.0 g, 57.5 mmol) in THF (180 mL) under nitrogen atmosphere was added Pd/C (3.40 g, 10 wt. %). The flask was evacuated and backfilled with H₂ (15 psi, 5 times). The resulting mixture was stirred for 6 h at RT under H₂ (15 psi). The suspension was then filtered through a pad of Celite®. The filtered cake was rinsed with THF (400 mL). The filtrate was concentrated under reduced pressure to provide (2S,3S)-2-((((9H-fluoren-9-yl)methoxy)carbonyl)amino)-3-(4-(tert-butoxycarbonyl)phenyl)butanoic acid. MS ESI calculated for C₃₀H₃₁NO₆ [M+Na]⁺ 524, found 524. ¹H NMR (400 MHz, DMSO-d₆): δ δ 7.82 (m, J=8.0, 7.6 Hz, 4H), 7.53-7.50 (m, 2H), 7.43-7.35 (m, 4H), 7.24 (q, J=8.4 Hz, 2H), 4.07-4.00 (m, 2H), 3.24-3.16 (m, 1H), 1.48 (s, 9H), 1.25 (d, J=7.2 Hz, 3H).

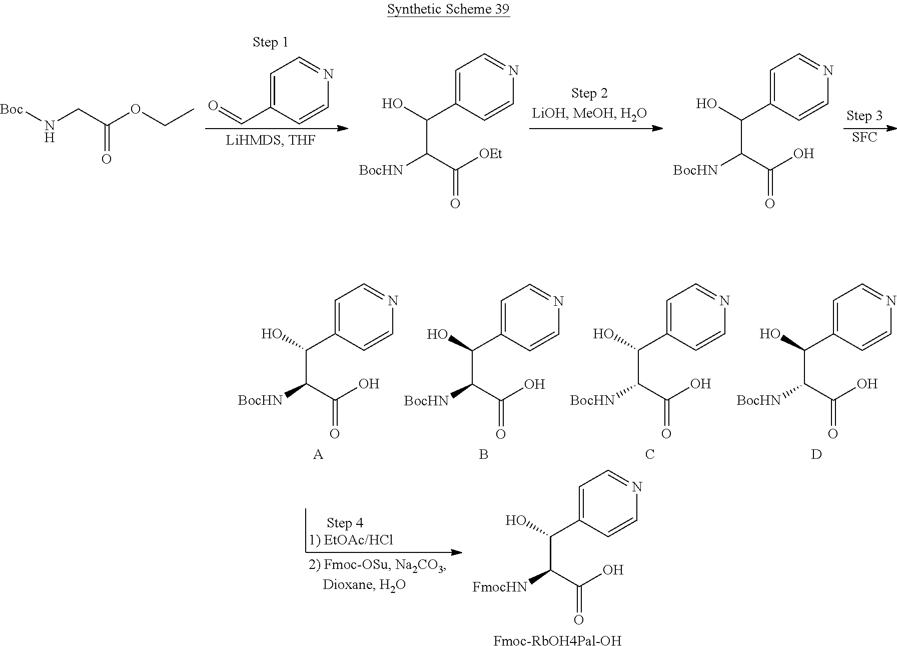

### (2S,3R)-2-((((9H-Fluoren-9-yl)methoxy)carbonyl)amino)-3-hydroxy-3-(pyridin-4-yl)propanoic acid (Fmoc-RbOH4Pal-OH)

Step 1: In a three necked flask, a solution of ethyl (tert-butoxycarbonyl)glycinate (100.0 g, 492 mmol) in anh. THF (1.5 L) was purged with N₂ three times and cooled at −60° C. A solution of LiHMDS (1.0 M, 1.01 L, 1010 mmol) in THF was added dropwise at −60° C. The reaction mixture was stirred at −65° C. for 1 h under N₂. Isonicotinaldehyde (69.3 mL, 79.1 g, 738 mmol) was added to the mixture at −60° C. The reaction mixture was allowed to warm to RT and was stirred at RT for 4 h. The reaction mixture was quenched with aq. sat. NH₄Cl (1 L) and extracted with EtOAc (2×1 L). The). The combined organic layer were washed with brine, dried over anh. Na₂SO₄, filtered, and concentrated in vacuum. The residue was purified by silica gel chromatography, eluting with EtOAc 2-100% in PE to afford ethyl 2-((tert-butoxycarbonyl) amino)-3-hydroxy-3-(pyridin-4-yl)propanoate. MS ESI calculated for C₁₅H₂₂N₂O₅ [M+H]⁺ 311, found 311. ¹H NMR (400 MHz, methanol-d₄): δ 8.49-8.48 (m, 2H), 7.49-7.48 (m, 2H), 5.38-4.91 (m, 1H), 4.55-4.38 (m, 1H), 4.34-4.04 (m, 2H), 1.39-1.27 (m, 9H), 1.26-1.13 (m, 3H).

Step 2: A solution of LiOH·H₂O (53.8 g, 1.28 mol) and ethyl 2-((tert-butoxycarbonyl) amino)-3-hydroxy-3-(pyridin-4-yl)propanoate (82.0 g, 0.25 mmol) in MeOH (640 mL) and H₂O (160 mL) was stirred at RT for 2 h. The reaction mixture was concentrated in vacuo and the residue was purified by prep-HPLC (water(HCl)-MeCN]; MeCN %: 1-27%, 20 min) to afford 2-((tert-butoxycarbonyl)amino)-3-hydroxy-3-(pyridin-4-yl)propanoic acid. MS ESI calculated for C₁₃H₁₈N₂O₅ [M+H]⁺ 283, found 283.

Step 3: The mixture of four isomers was separated by SFC(SFC (column: Daicel ChiralPak® AD (250×50 mm, 10 μm, Daicel Chiral Technologies, West Chester, PA); mobile phase: [0.1% NH₄OH in H₂O; IPA]; IPA %: 30-30%, 7 min)) to obtain compounds A, B, C, and D.

Step 4: (2S,3R)-2-((tert-Butoxycarbonyl)amino)-3-hydroxy-3-(pyridin-4-yl)propanoic acid (Compound A, 10.0 g, 35.4 mmol) was dissolved in a solution of hydrogen chloride in EtOAc (4.0 M, 71.9 mL, 287.6 mmol) and the solution was stirred at RT for 2 h. The pH of the reaction mixture was adjusted to ˜3-4 by slow addition of an aqueous solution of NaHCO₃ (41.6 g, 496 mmol, 19.3 mL) at 0° C. The aqueous layer was isolated and used for the next step.

Fmoc-OSu (16.7 g, 49.6 mmol) in dioxane (60 mL) was added to the aqueous solution. The reaction mixture was stirred at RT for 2 h and then concentrated in vacuo. The residue was purified by Rp HPLC (column: Welch Xtimate® C18 250×70 mm (Welch, West Haven, CT), 10 μm; mobile phase: [water with NH₄HCO₃; MeCN]; MeCN %: 1-40%, 20 min) to afford (2S,3R)-2-((((9H-fluoren-9-yl)methoxy)carbonyl)amino)-3-hydroxy-3-(pyridin-4-yl)propanoic acid. MS ESI calculated for C₂₃H₂₀N₂O₅ [M+H]⁺ 405, found 405. ¹HNMR (400 MHz, DMSO-d₆): δ 12.95 (s, 1H), 8.51-8.45 (m, 2H), 7.88 (d, J=7.4 Hz, 2H), 7.62 (dd, J=20.7, 7.3 Hz, 2H), 7.46-7.37 (m, 4H), 7.35-7.24 (m, 3H), 5.96 (s, 1H), 5.21 (s, 1H), 4.41 (d, J=9.5 Hz, 1H), 4.15-4.03 (m, 3H).

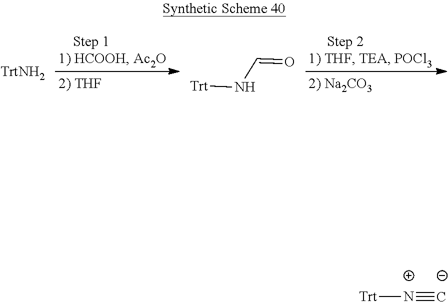

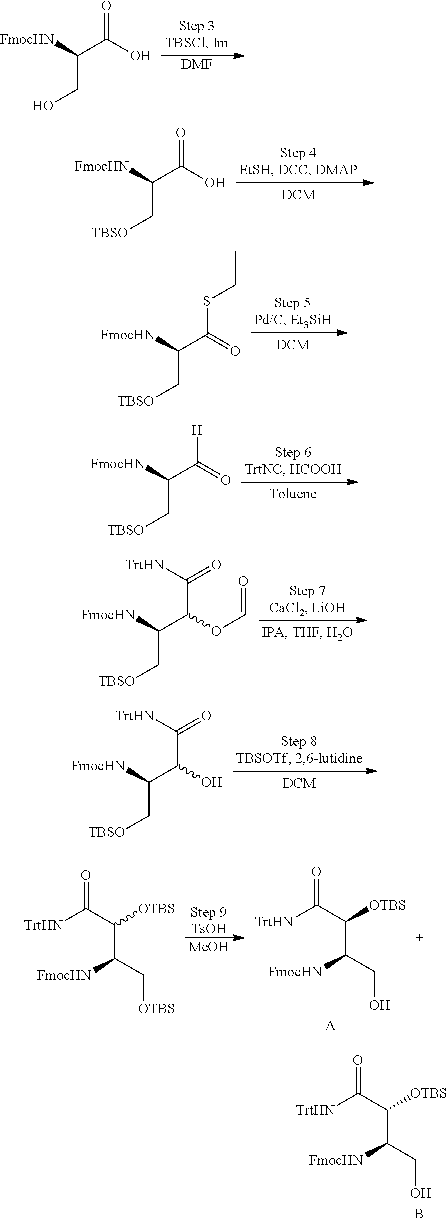

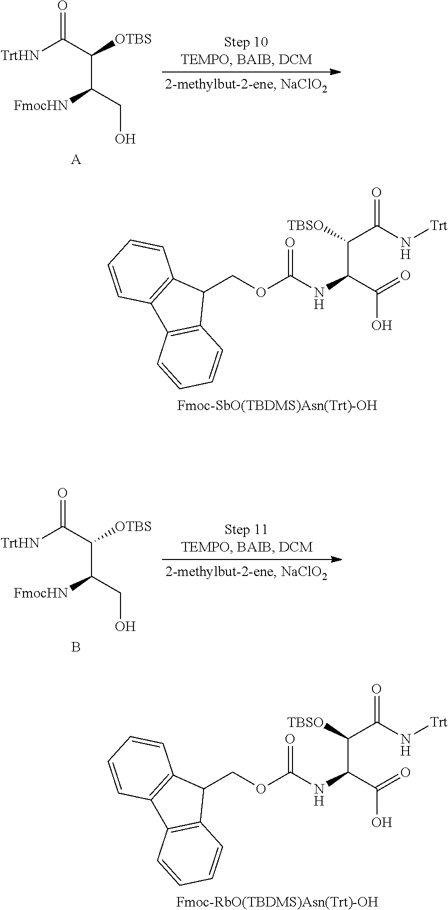

### (2S,3S)-2-((((9H-Fluoren-9-yl)methoxy)carbonyl)amino)-3-((tert-butyldimethylsilyl)oxy)-4-oxo-4-(tritylamino)butanoic acid (Fmoc-SbO(TBDMS)Asn(Trt)OH) and (2S,3R)-2-((((9H-fluoren-9-yl)methoxy)carbonyl)amino)-3-((tert-butyldimethylsilyl)oxy)-4-oxo-4-(tritylamino)butanoic acid (Fmoc-RbO(TBDMS)Asn(Trt)OH)

Step 1: Formic acid (1.9 mL, 50.1 mmol) and Ac₂O (4.7 mL, 50.1 mmol) were stirred in a dry flask at 55° C. for 2 h. The solution was then added dropwise to a solution of triphenylmethanamine (13.0 g, 50.1 mmol) in anh. THF (84 mL) at 0° C. under N₂ atmosphere. The reaction mixture was warmed to RT and stirred for 2 h. The resulting solution was quenched with an aq. sat. Na₂CO₃ solution and extracted with EtOAc (200 mL). The organic layer was washed with brine, dried over anh. Na₂SO₄, filtered, and concentrated in vacuum to afford N-tritylformamide, which was used in Step 2 without further purification. ¹H NMR (300 MHz, DMSO-d₆): δ 9.01 (s, 1H), 8.13 (d, J=1.9 Hz, 1H), 7.41-7.10 (m, 15H).

Step 2: A solution of N-tritylformamide (15.3 g, 53.2 mmol) and TEA (21.5 mL, 154 mmol) in anh. THF (96 mL) was cooled to 0° C. under N₂ atmosphere. POCl₃ (8.44 mL, 91 mmol) was added dropwise to the reaction mixture and stirred for 1 h. The resulting solution was quenched with an aq. sat. Na₂CO₃ solution (180 mL) and extracted with DCM (3×200 mL). The combined organic layers were dried over anh. Na₂SO₄, filtered, and concentrated in vacuum. The residue was purified by silica gel chromatography, eluting with EtOAc 0-7% in PE to afford (isocyanomethanetriyl)tribenzene. ¹H NMR (300 MHz, DMSO-d₆): δ 7.54-7.10 (m, 15H).

Step 3: Imidazole (6.2 g, 92 mmol) and TBSCl (6.9 g, 45.8 mmol) at RT and under N₂, were added to a solution of (((9H-fluoren-9-yl)methoxy)carbonyl)-D-serine (10.0 g, 30.5 mmol) in anh. DMF (100 mL). The reaction was stirred at RT for 12 h. The resulting solution was quenched with 1 M HCl and extracted with EtOAc (3×300 mL). The combined organic layers were washed with brine (2×150 mL), dried over anh. Na₂SO₄, and filtered. The filtrate was concentrated in vacuum and the residue was purified by silica gel chromatography, eluting with MeOH 0-5% in DCM to afford N-(((9H-fluoren-9-yl)methoxy)carbonyl)-O-(tert-butyldimethylsilyl)-D-serine. MS ESI calculated for C₂₄H₃₂NO₅Si [M+H]⁺ 442.15, found 442.20. ¹H NMR (300 MHz, chloroform-d): δ 7.76 (d, J=7.5 Hz, 2H), 7.63-7.59 (m, 2H), 7.44-7.27 (m, 4H), 5.63 (d, J=8.2 Hz, 1H), 4.53-4.32 (m, 3H) 4.25 (t, J=7.2 Hz, 1H), 4.16-4.13 (m, 1H), 3.90-3.85 (m, 1H), 0.90 (s, 9H), 0.08 (d, J=2.6 Hz, 6H).

Step 4: DMAP (0.373 g, 3.06 mmol) at RT under N₂ was added to a solution of N-(((9H-fluoren-9-yl)methoxy)carbonyl)-O-(tert-butyldimethylsilyl)-D-serine (13.5 g, 30.6 mmol) in DCM (150 mL). The reaction was cooled to 0° C. DCC (12.6 g, 61.1 mmol) and ethanethiol (2.9 g, 45.9 mmol) were added to the mixture. The reaction was warmed to RT and stirred for 4 h. The resulting solution was filtered through a Celite® pad. The filtrate was concentrated under reduced pressure and the residue was purified by silica gel chromatography, eluting with EtOAc 0-12% in PE to afford S-ethyl (R)-2-((((9H-fluoren-9-yl)methoxy)carbonyl)amino)-3-((tert-butyldimethylsilyl)oxy)propanethioate. MS ESI calculated for C₂₆H₃₆NO₄SSi [M+H]⁺ 486.21, found 486.25. ¹H NMR (300 MHz, chloroform-d): δ 7.76-7.22 (m, 8H), 4.53-3.96 (m, 5H), 3.76 (dd, J=10.2, 3.6 Hz, 1H), 2.93-2.75 (m, 2H), 1.21 (t, J=7.5 Hz, 3H), 0.85 (s, 9H), 0.01 (d, J=6.3 Hz, 6H).

Step 5: Pd/C (2.5 g, 2.35 mmol, dry, 10 wt. %) and triethylsilane (6.32 g, 54.4 mmol) under Ar were added to a solution of S-ethyl (R)-2-((((9H-fluoren-9-yl)methoxy)carbonyl) amino)-3-((tert-butyldimethylsilyl)oxy)propanethioate (13.2 g, 27.2 mmol) in DCM (130 mL). The reaction mixture was stirred at RT for 4 h. The resulting solution was filtered through a pad of Celite®. The filtrate was concentrated in vacuum and the residue was purified by silica gel chromatography, eluting with EtOAc 0-23% in PE to afford (9H-fluoren-9-yl)methyl (R)-(1-((tert-butyldimethylsilyl)oxy)-3-oxopropan-2-yl)carbamate. MS ESI calculated for C₂₄H₃₂NO₄Si [M+H]⁺ 426.20, found 426.05. ¹H NMR (300 MHz, chloroform-d): δ 9.61 (s, 1H), 7.71 (d, J=7.5 Hz, 2H), 7.60-7.52 (m, 2H), 7.40-7.22 (m, 4H), 4.36 (d, J=7.1 Hz, 2H), 4.32-4.24 (m, 1H), 4.24-4.13 (m, 2H), 3.90-3.79 (m, 1H), 0.82 (s, 9H). 0.04 (s, 6H).

Step 6: (Isocyanomethanetriyl)tribenzene (7.0 g, 26.0 mmol) at RT was added to a solution of (9H-fluoren-9-yl)methyl (R)-(1-((tert-butyldimethylsilyl)oxy)-3-oxopropan-2-yl) carbamate (8.5 g, 19.97 mmol) in toluene (80 mL). Formic acid (1.5 mL, 39.9 mmol) was added dropwise. The reaction mixture was warmed to 60° C. and stirred o.n. at 60° C. The resulting solution was quenched with an aq. sat. Na₂CO₃ solution and extracted with EtOAc (2×200 mL). The combined organic layers were dried over anh. Na₂SO₄, filtered, and concentrated under reduced pressure. The residue was purified by silica gel chromatography, eluting with EtOAc 0-15% in PE to afford (3R)-3-((((9H-fluoren-9-yl)methoxy)carbonyl)amino)-4-((tert-butyldimethylsilyl)oxy)-1-oxo-1-(tritylamino)butan-2-yl formate. MS ESI calculated for C₄₅H₄₉N₂O₆Si [M+H]⁺ 741.33, found 741.20. ¹H NMR (300 MHz, chloroform-d): δ 8.13 (s, 1H), 7.73 (d, J=7.5 Hz, 2H), 7.59-7.48 (m, 2H), 7.41-7.31 (m, 2H), 7.31-7.11 (m, 19H), 5.49 (s, 1H), 4.45-4.21 (m, 3H), 4.18-4.14 (m, 1H), 3.64-3.62 (m, 1H), 3.59-3.47 (m, 1H), 0.87-0.81 (m, 9H), 0.07-0.01 (m, 6H).

Step 7: CaCl₂ (20.7 g, 186 mmol) at RT was added to a solution of (3R)-3-((((9H-fluoren-9-yl)methoxy)carbonyl)amino)-4-((tert-butyldimethylsilyl)oxy)-1-oxo-1-(tritylamino)butan-2-yl formate (9.2 g, 12.42 mmol) in 2-propanol (188 mL) and THF (94 mL). After CaCl₂ was dissolved by sonication, a solution of LiOH (1.2 g, 49.7 mmol) in water (47 mL) was added dropwise. The reaction was stirred at RT for 2 h. The reaction mixture was acidified by addition of 0.1 N HCl to pH ˜5. The mixture was partially concentrated under vacuum to remove most of isopropanol and THF. The mixture was diluted with H₂O (150 mL) and extracted with DCM (3×300 mL). The combined organic layers were dried over anh. Na₂SO₄, filtered, concentrated under vacuum, and purified by flash column chromatography on silica gel, eluting with EtOAc 0-25% in PE to give (9H-fluoren-9-yl)methyl ((2R)-1-((tert-butyldimethylsilyl)oxy)-3-hydroxy-4-oxo-4-(tritylamino)butan-2-yl)carbamate. MS ESI calculated for C₄₄H₄₈N₂O₅SiNa [M+Na]⁺ 736.33, found 736.35.

Step 8: tert-Butyldimethylsilyl trifluoromethanesulfonate (2.8 g, 10.5 mmol) at 0° C. was added dropwise to a solution of (9H-fluoren-9-yl)methyl ((2R)-1-((tert-butyldimethylsilyl)oxy)-3-hydroxy-4-oxo-4-(tritylamino)butan-2-yl)carbamate (7.5 g, 10.5 mmol) in DCM (75 mL). The reaction was stirred at 0° C. for 1 h. The mixture was concentrated in vacuum and the residue was purified by silica gel chromatography, eluting with EtOAc 0-35% in PE to afford (9H-fluoren-9-yl)methyl ((6R)-2,2,3,3,9,9,10,10-octamethyl-5-(tritylcarbamoyl)-4,8-dioxa-3,9-disilaundecan-6-yl)carbamate. MS ESI calculated for C₅₀H₆₃N₂O₅Si₂ [M+H]⁺ 827.42, found 827.35. ¹H NMR (400 MHz, chloroform-d): δ 7.98 (s, 1H), 7.79-7.76 (m, 2H), 7.62-7.58 (m, 2H), 7.46-7.36 (m, 2H), 7.34-7.19 (m, 17H), 4.99-4.83 (m, 1H), 4.50-4.32 (m, 3H), 4.24 (t, J=7.0 Hz, 1H), 4.03-3.94 (m, 1H), 3.87-3.56 (m, 2H), 0.90 (d, J=11.4 Hz, 18H), 0.23-0.08 (m, 6H), 0.03 (s, 6H).

Step 9: A solution of p-toluenesulfonic acid monohydrate (1.7 g, 8.7 mmol) in MeOH (68 mL) was added dropwise to a mixture of (9H-fluoren-9-yl)methyl ((6R)-2,2,3,3,9,9,10,10-octamethyl-5-(tritylcarbamoyl)-4,8-dioxa-3,9-disilaundecan-6-yl)carbamate (7.2 g, 8.7 mmol) in MeOH (270 mL). The reaction mixture was stirred at RT for 1 h. The reaction mixture was concentrated under vacuum and purified by flash column chromatography on silica gel, eluting with EtOAc 0-9% in DCM to give both diastereoisomers (A and B).

(9H-Fluoren-9-yl)methyl ((2R,3S)-3-((tert-butyldimethylsilyl)oxy)-1-hydroxy-4-oxo-4-(tritylamino)butan-2-yl)carbamate (A). MS ESI calculated for C₄₄H₄₉N₂O₅Si [M+H]⁺ 713.33, found 713.30. ¹H NMR (400 MHz, chloroform-d): δ 8.19 (s, 1H), 7.80-7.70 (m, 2H), 7.60-7.53 (m, 2H), 7.44-7.19 (m, 19H), 4.91 (d, J=9.2 Hz, 1H), 4.54-4.30 (m, 3H), 4.24 (t, J=6.7 Hz, 1H), 4.04-4.03 (m, 1H), 3.81-3.78 (m, 1H), 3.52-3.50 (m, 1H), 0.92 (s, 9H), 0.19 (d, J=11.8 Hz, 6H).

(9H-Fluoren-9-yl)methyl ((2R,3R)-3-((tert-butyldimethylsilyl)oxy)-1-hydroxy-4-oxo-4-(tritylamino)butan-2-yl)carbamate (B). MS ESI calculated for C₄₄H₄₉N₂O₅Si [M+H]⁺ 713.33, found 713.30. ¹H NMR (400 MHz, chloroform-d): δ 8.02 (s, 1H), 7.75 (d, J=7.5 Hz, 2H), 7.56 (d, J=7.8 Hz, 2H), 7.45-7.11 (m, 19H), 4.45-4.12 (m, 4H), 4.00-3.99 (s, 1H), 3.69-3.59 (m, 2H), 0.88 (s, 9H), 0.18 (s, 3H), 0.10 (s, 3H).

Step 10: Phenyl-λ³-iodanediyl diacetate (2.35 g, 7.29 mmol) and TEMPO (0.11 g, 0.73 mmol) at 0° C. were added to a mixture of (9H-fluoren-9-yl)methyl ((2R,3S)-3-((tert-butyldimethylsilyl)oxy)-1-hydroxy-4-oxo-4-(tritylamino)butan-2-yl)carbamate (A) (2.6 g, 3.65 mmol) in DCM (26 mL). The reaction was stirred at 0° C. for 2 h. Sodium chlorite (0.66 g, 7.29 mmol) and 2-methylbut-2-ene (0.51 g, 7.29 mmol) were added to the mixture at 0° C. The reaction mixture was stirred o.n. at RT. The reaction mixture was diluted with EtOAc (100 mL), washed with aq. sat. Na₂S₂O₃ (2×50 mL), brine (2×50 mL). The organic phase was dried over anh. Na₂SO₄, concentrated under vacuum and purified by flash column chromatography on silica gel, eluting with EtOAc 0-50% in DCM to afford (2S,3S)-2-((((9H-fluoren-9-yl)methoxy) carbonyl)amino)-3-((tert-butyldimethylsilyl)oxy)-4-oxo-4-(tritylamino)butanoic acid. MS ESI calculated for C₄₄H₄₇N₂O₆Si [M+H]⁺ 727.31, found 727.30. ¹H NMR (300 MHz, methanol-d₄): δ 7.79-7.74 (m, 2H), 7.65-7.60 (m, 2H), 7.41-7.16 (m, 19H), 4.59-4.58 (m, 2H), 4.48-4.31 (m, 2H), 4.22 (t, J=6.6 Hz, 1H), 0.83 (s, 9H), 0.10 (s, 3H), 0.01 (s, 3H).

Step 11: Phenyl-λ³-iodanediyl diacetate (2.08 g, 6.45 mmol) and TEMPO (0.10 g, 0.65 mmol) at 0° C. were added to a mixture of (9H-fluoren-9-yl)methyl ((2R,3R)-3-((tert-butyldimethylsilyl)oxy)-1-hydroxy-4-oxo-4-(tritylamino)butan-2-yl)carbamate (B) (2.3 g, 3.23 mmol) in DCM (26 mL). The reaction was stirred at 0° C. for 2 h. Sodium chlorite (0.58 g, 6.45 mmol) and 2-methylbut-2-ene (0.45 g, 6.45 mmol) were added to the mixture at 0° C. The reaction mixture was stirred o.n. at RT. The reaction mixture was diluted with EtOAc (100 mL), washed with aq. sat. Na₂S₂O₃ (2×50 mL), brine (2×50 mL). The organic phase was dried over anh. Na₂SO₄, concentrated under vacuum, and purified by flash column chromatography on silica gel, eluting with EtOAc 0-50% in DCM to afford (2S,3R)-2-((((9H-fluoren-9-yl)methoxy) carbonyl)amino)-3-((tert-butyldimethylsilyl)oxy)-4-oxo-4-(tritylamino)butanoic acid. MS ESI calculated for C₄₄H₄₇N₂O₆Si [M+H]⁺ 727.31, found 727.30. ¹H NMR (300 MHz, methanol-d₄): δ 7.72-7.53 (m, 3H), 7.48 (d, J=7.5 Hz, 1H), 7.31-7.21 (m, 2H), 7.17-6.98 (m, 17H), 4.69-4.59 (m, 2H), 4.43-4.33 (m, 1H), 4.24-4.13 (m, 1H), 4.09 (t, J=6.9 Hz, 1H), 0.75 (s, 9H), 0.04-0.00 (m, 6H).

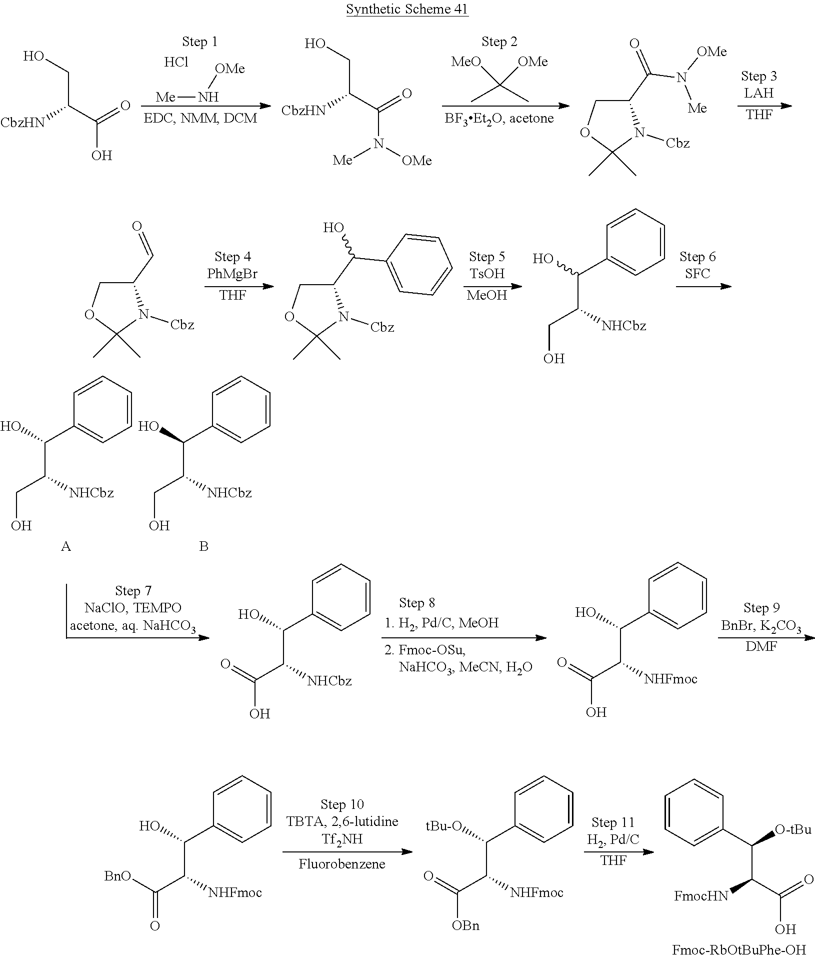

### (2S,3R)-2-((((9H-Fluoren-9-yl)methoxy)carbonyl)amino)-3-hydroxy-3-phenylpropanoic acid (Fmoc-RbOtBuPhe-OH)

Step 1: N,O-Dimethylhydroxylamine hydrochloride (104 g, 1.07 mol) and N-methylmorpholine (179 g, 1.78 mol, 195 mL) were added to a solution of ((benzyloxy)carbonyl)-D-serine (170 g, 710 mmol) in DCM (1.20 L) at −15° C. EDC (143 g, 746 mmol) was added to the mixture at −15° C. The reaction mixture was stirred at 0° C. for 4 h. The reaction mixture was quenched by addition of aq. 1 N HCl (2.00 L) to the reaction at −15° C. The organic layer was isolated, and the aqueous layer was extracted with DCM (1.00 L). The combined organic layers were washed with sat. NaHCO₃ (1.00 L), dried over anh. Na₂SO₄, filtered, and concentrated under reduced pressure to afford benzyl-(R)-(3-hydroxy-1-(methoxy(methyl)amino)-1-oxopropan-2-yl)carbamate. ¹H NMR (400 MHz DMSO-d₆): δ 7.83-7.30 (m, 5H), 5.02 (s, 2H), 4.88 (t, J=6.0 Hz, 1H), 4.60 (d, J=4.8 Hz, 1H), 3.72 (s, 3H), 3.60-3.56 (m, 1H), 3.51-3.46 (m, 2H), 3.11 (s, 3H).

Step 2: Crude benzyl-(R)-(3-hydroxy-1-(methoxy(methyl)amino)-1-oxopropan-2-yl)carbamate (204 g, 723 mmol) was dissolved in a mixture of acetone (880 mL) and 2,2-dimethoxypropane (693 g, 6.66 mol, 816 mL). BF₃·Et₂O (9.38 g, 66.1 mmol, 8.16 mL) was added to the solution. The reaction mixture was stirred at RT for 5 h. The reaction mixture was quenched by addition of triethylamine (10.0 mL). The volatiles were removed under reduced pressure. The resulting residue was purified by silica gel column chromatography, eluting with EtOAc in PE (10:1-3:1; v/v) to yield benzyl (R)-4-(methoxy(methyl)carbamoyl)-2,2-dimethyloxazolidine-3-carboxylate.

Step 3: A suspension of LAH (9.08 g, 239 mmol) in anh. THF (440 mL) was added to a solution of benzyl-(R)-4-(methoxy(methyl)carbamoyl)-2,2-dimethyloxazolidine-3-carboxylate (145 g, 450 mmol) in anh. THF (440 mL) at 0° C. The mixture was stirred at 0° C. for 1 h. The reaction mixture was quenched by addition of an aq. sat. solution of KHSO₄ (1.5 L) to the mixture at −10° C. MTBE (1.0 L) was added, and the mixture was stirred at 0° C. for 30 min. The solids were removed by filtration. The organic layer was isolated, dried over anh. Na₂SO₄, filtered and concentrated under reduced pressure. The residue was purified by silica gel column chromatography, eluting with EtOAc in PE (10:1-2:1; v/v) to yield benzyl (R)-4-formyl-2,2-dimethyloxazolidine-3-carboxylate. ¹H NMR (400 MHz, DMSO-d₆): δ 9.57 (s, 1H), 7.39-7.31 (m, 5H), 5.15-4.99 (m, 2H), 4.49-4.11 (m, 1H), 3.95-3.90 (m, 2H), 1.54-1.42 (m, 6H).

Step 4: Phenylmagnesium bromide (3.0 M in diethyl ether, 60.7 mL, 180 mmol) was diluted into anh. THF (200 mL) and the solution was cooled to 0° C. A solution of benzyl (R)-4-formyl-2,2-dimethyloxazolidine-3-carboxylate (40.0 g, 152 mmol) in anh. THF (50.0 mL) was added to the reaction mixture at 0° C. The resulting solution was stirred at RT for 2 h. The reaction mixture was slowly poured into an iced-cold aq. sat. NH₄C₁ solution (500 mL) at 0° C. and extracted with MTBE (2×250 mL). The combined organic layers were washed with brine (300 mL), dried over anh. Na₂SO₄, filtered, and concentrated under reduced pressure to afford crude benzyl (4R)-4-(hydroxy(phenyl)methyl)-2,2-dimethyloxazolidine-3-carboxylate, which was used in Step 5 without further purification.

Step 5: p-Toluenesulfonic acid monohydrate (11.1 g, 58.6 mmol) was added to a solution of benzyl (4R)-4-(hydroxy(phenyl)methyl)-2,2-dimethyloxazolidine-3-carboxylate (40.0 g, 117 mmol) in MeOH (250 mL). The solution was stirred at 50° C. for 12 h. The reaction mixture was quenched by slow addition of an aq. sat. NaHCO₃ solution (500 mL) and then extracted with DCM (2×350 mL). The combined organic layers were dried over anh. Na₂SO₄, filtered, and concentrated under reduced pressure. The residue was purified by silica gel column chromatography, eluting with EtOAc in PE (6:1-0:1; v/v) to yield benzyl ((2R)-1,3-dihydroxy-1-phenylpropan-2-yl)carbamate.

Step 6: The mixture of diastereomers was separated by SFC: (column: Daicel ChiralPak® IC (250×50 mm, 10 μm, Daicel Chiral Technologies, West Chester, PA); mobile phase: [0.1% NH₄OH in H₂O; IPA]; IPA %: 40-40%, 5 min).

Benzyl ((1R,2R)-1,3-dihydroxy-1-phenylpropan-2-yl)carbamate A. ¹H NMR (400 MHz, DMSO-d₆): δ 7.35-7.18 (m, 10H), 6.94 (d, J=8.2 Hz, 1H), 5.38 (d, J=5.6 Hz, 1H), 4.90 (dd, J=13.2 Hz, 28.8 Hz, 2H), 4.57-4.47 (m, 2H), 3.63-3.53 (m, 3H).

Benzyl ((1S,2R)-1,3-dihydroxy-1-phenylpropan-2-yl)carbamate B. ¹H NMR (400 MHz, DMSO-d₆): δ 7.35-7.21 (m, 10H), 6.65 (d, J=8.2 Hz, 1H), 5.32 (d, J=5.6 Hz, 1H), 4.93 (dd, J=13.2 Hz, 28.8 Hz, 2H), 4.81-4.72 (m, 2H), 3.68-3.67 (m, 1H), 3.51-3.49 (m, 1H), 3.33-3.28 (m, 1H).

Step 7: Sodium hypochlorite (10.5 g, 14.1 mmol, 8.65 mL, 10% active chlorine basis) was added to a solution of benzyl ((1R,2R)-1,3-dihydroxy-1-phenylpropan-2-yl)carbamate A (18.0 g, 56.3 mmol, 94.2% purity) and TEMPO (1.77 g, 11.3 mmol) in acetone (90.0 mL) and aq. NaHCO₃(194 g, 116 mmol, 90.0 mL, 5 wt. %). The reaction mixture was stirred at RT for 1 h under N₂. The reaction mixture was diluted with DI water (300 mL) and extracted with EtOAc (2×150 mL). The pH of the aqueous layer was adjusted to pH ˜2 by addition of 1 N HCl solution. The mixture was extracted with EtOAc (2×150 mL), and the combined organic layers were dried over anh. Na₂SO₄, filtered, and concentrated under reduced pressure to give crude (2S,3R)-2-(((benzyloxy)carbonyl)amino)-3-hydroxy-3-phenylpropanoic acid, which was used in Step 8 without further purification. ¹H NMR (400 MHz, DMSO-d₆): δ 12.8 (s, 1H), 7.94-7.92 (m, 1H), 7.92-7.39 (m, 1H), 7.38-7.322 (m, 10H), 7.1 (m, 1H), 5.67 (s, 1H), 5.13-5.11 (m, 1H), 5.12 (s, 2H), 4.31 (dd, J=9.6 Hz, 3.2 Hz, 1H).

Step 8: Crude (2S,3R)-2-(((benzyloxy)carbonyl)amino)-3-hydroxy-3-phenylpropanoic acid (12.0 g, 38.0 mmol) was added to a mixture of aq. 12 M HCl solution (3.16 mL) in MeOH (60.0 mL). Pd/C (6.00 g, 10 wt. %) was added to the solution under N₂. The flask was evacuated and backfilled with H₂ (5 times). The resulting mixture was stirred for 3 h at RT under an atmosphere of dihydrogen (15 psi). The flask was evacuated and refilled with N₂ (5 times). The resulting mixture was filtered. Filter cake was washed with MeOH (2×50 mL). The filtrate was concentrated under reduced pressure. To the crude solid were added MeCN (40.0 mL) and H₂O (8.00 mL) at RT, followed by NaHCO₃ (6.39 g, 76.1 mmol) and Fmoc-OSu (10.3 g, 30.4 mmol). The reaction mixture was stirred at RT for 2 h. Upon completion of the Fmoc-protection, the pH of the mixture was adjusted to 4 by addition of aq. 0.5 N HCl solution. The reaction mixture was extracted with EtOAc (2×150 mL). The combined organic layers were dried over anh. Na₂SO₄, filtered, and concentrated under reduced pressure to give crude (2S,3R)-2-((((9H-fluoren-9-yl) methoxy)carbonyl)amino)-3-hydroxy-3-phenylpropanoic acid. ¹H NMR (400 MHz, DMSO-d₆): δ 12.8 (s, 1H), 7.87 (d, J=7.6 Hz, 2H), 7.42 (d, J=24.4 Hz, 2H), 7.43-7.26 (m, 11H), 5.76 (s, 1H), 5.16 (s, 1H), 4.28 (dd, J=9.6 Hz, 3.6 Hz, 1H), 4.14-4.07 (m, 3H).

Step 9: To a solution of crude (2S,3R)-2-((((9H-fluoren-9-yl)methoxy)carbonyl)amino)-3-hydroxy-3-phenylpropanoic acid (1.00 g, 2.48 mmol) in DMF (7.00 mL) was added K₂CO₃ (685 mg, 4.96 mmol) followed by benzyl bromide (508 mg, 2.97 mmol, 353 μL) dropwise at RT. The suspension was stirred at RT for 3 h. Upon completion of the benzyl-protection, the suspension was diluted into an aq. sat. NH₄Cl solution (50.0 mL) at RT and extracted with EtOAc (2×50.0 mL). The combined organic layers were washed with brine (50.0 mL), dried over anh. Na₂SO₄, filtered, and concentrated under reduced pressure. The crude residue was triturated with PE (10.0 mL) at RT for 15 min and filtered to afford benzyl (2S,3R)-2-((((9H-fluoren-9-yl)methoxy) carbonyl)amino)-3-hydroxy-3-phenylpropanoate. MS ESI calculated for C₃₁H₂₇NO₅ [M+H]⁺ 494, found 494. ¹H NMR (400 MHz, chloroform-d): δ 7.78 (d, J=7.2 Hz, 2H), 7.56 (m, 2H), 7.42-7.16 (m, 15H), 5.53 (d, J=7.6 Hz, 1H), 5.23 (s, 1H), 5.15-5.13 (m, 2H), 4.86-4.84 (m, 1H), 4.53-4.48 (m, 2H), 4.38 (t, J=6.8 Hz, 1H), 4.24 (t, J=6.8 Hz, 1H).

Step 10: Bis(trifluoromethane)sulfonimide (71.2 mg, 253 mmol) and 2,6-lutidine (54.2 mg, 506 mmol, 59.0 μL) were added to a solution of benzyl-(2S,3R)-2-((((9H-fluoren-9-yl) methoxy)carbonyl)amino)-3-hydroxy-3-phenylpropanoate (500 mg, 1.01 mmol) in fluorobenzene (1.00 mL) at RT under N₂. tert-Butyl 2,2,2-trichloroacetimidate (1.11 g, 5.07 mmol, 907 μL) was added dropwise at RT under N₂. The mixture was stirred at RT for 72 h. The suspension was filtered through a pad of silica gel and the filtered cake was rinsed with DCM (2×20.0 mL). The filtrate was concentrated under vacuum. The crude product was purified by Rp HPLC (column: Phenomenex™ Luna™ C18, 80×40 mm, 3 μm, (Phenomenex™, Torrance, CA); mobile phase: [water(NH₄CO₃H)-MeCN]; MeCN %: 50-80%, 8 min) to yield benzyl (2S,3R)-2-((((9H-fluoren-9-yl)methoxy)carbonyl)amino)-3-(tert-butoxy)-3-phenylpropanoate. ¹H NMR (400 MHz, chloroform-d): δ 7.78 (d, J=7.2 Hz, 2H), 7.59 (d, J=7.2 Hz, 2H), 7.40 (t, J=7.2 Hz, 2H), 7.35-7.23 (m, 12H), 5.54 (d, J=8.8 Hz, 1H), 5.10 (s, 2H), 4.94 (d, J=4.0 Hz, 1H), 4.67-4.65 (m, 1H), 4.43-4.22 (m, 3H), 1.13 (s, 9H).

Step 11: Pd/C (32.0 mg, 10 wt. %) under N₂ was added to a solution of benzyl (2S,3R)-2-((((9H-fluoren-9-yl)methoxy)carbonyl)amino)-3-(tert-butoxy)-3-phenylpropanoate (310 mg, 564 μmol) in THF (3.00 mL). The flask was evacuated and backfilled with H₂ (3 times). The resulting mixture was stirred for 12 h at RT under an atmosphere of hydrogen gas (15 psi). The flask was evacuated and refilled with N₂ (5 times). The resulting mixture was filtered through a pad of silica gel and the filtered cake was rinsed with THF (2×20 mL). The filtrate was concentrated under reduced pressure to give (2S,3R)-2-((((9H-fluoren-9-yl)methoxy)carbonyl)amino)-3-(tert-butoxy)-3-phenylpropanoic acid. MS ESI calculated for C₂₈H₂₉NO₅ [M+Na]: 482, found 482. ¹H NMR (400 MHz, chloroform-d): δ 7.76 (d, J=7.6 Hz, 2H), 7.55-7.53 (m, 2H), 7.42-7.27 (m, 9H), 5.41 (s, 1H), 4.96 (s, 1H), 4.56 (s, 1H), 4.37-4.12 (m, 3H), 1.16 (s, 9H).

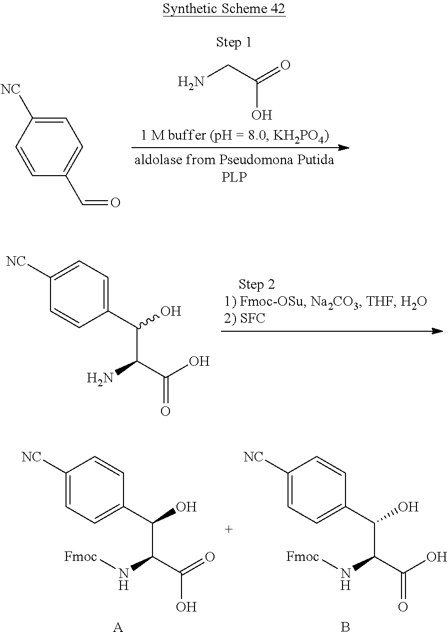

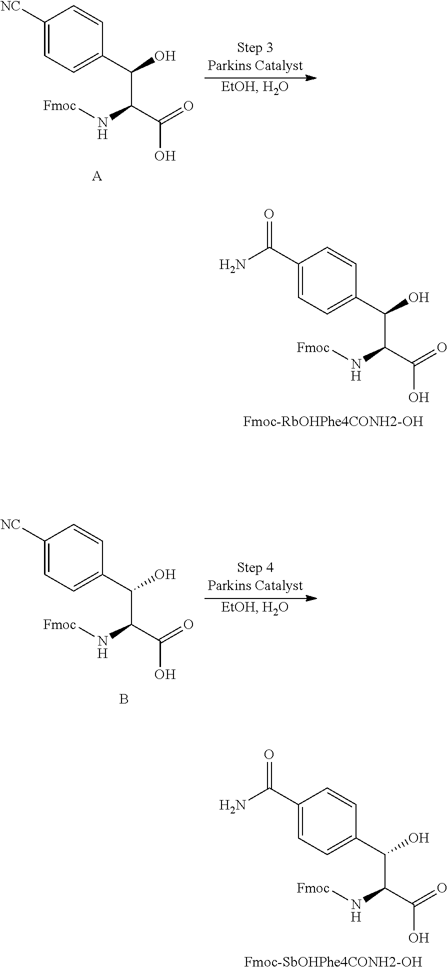

### (2S,3R)-2-((((9H-Fluoren-9-yl)methoxy)carbonyl)amino)-3-(4-carbamoylphenyl)-3-hydroxypropanoic acid (Fmoc-RbOHPhe4CONH2-OH) and (2S,3S)-2-((((9H-fluoren-9-yl)methoxy)carbonyl)amino)-3-(4-carbamoylphenyl)-3-hydroxypropanoic acid (Fmoc-SbOHPhe4CONH2-OH)

Step 1: Pyridoxal phosphate (15 mg, 0.061 mmol) and aldolase from pseudomona putida (1.0 g, 15.3 mmol) at RT, were added to a stirred solution of glycine (2.3 g, 30.5 mmol) in 1 M phosphate buffer (20 mL, pH=8.0). 4-Formylbenzonitrile (2.0 g, 15.3 mmol) then was added to the mixture at RT. The resulting mixture was stirred at RT for 20 h. The solution of (2S,3R)-2-amino-3-(4-cyanophenyl)-3-hydroxypropanoic acid was directly used in Step 2. MS ESI calculated for C₁₀H₁₁N₂O₃ [M+H]⁺ 207.07, found 207.15.

Step 2: Na₂CO₃ (2.67 g, 25.2 mmol) and Fmoc-OSu (8.51 g, 25.2 mmol) at RT were added to a stirred solution of (2S,3R)-2-amino-3-(4-cyanophenyl)-3-hydroxypropanoic acid (2.6 g, 12.61 mmol) in THF (38 mL) and water (38 mL). The resulting mixture was stirred o.n. at RT. The pH was adjusted to ˜3 with 1 N HCl. The resulting mixture was extracted with EtOAc (500 mL), washed with brine (3×200 mL), and dried over anh. Na₂SO₄. The solvent was concentrated under reduced pressure and the residue was purified by Rp-flash column chromatography with the following conditions: Column: Flash C18 (330 g); Mobile Phase A: water (0.05% TFA), Mobile Phase B: MeCN; Gradient Elution: 2-70%; Detector: UV 210 nm. The fractions containing the product were concentrated under reduced pressure to afford a mixture of diasteromeric products. The mixture of diastereomers was separated by SFC: (column: CHIRAL ART® Amylose-C (250×50 mm, 5 μm); mobile phase: [0.1% 2 M NH₃-MeOH; MeOH]; MeOH %: 50-50%, 10 min).

The faster peak was obtained at 4.49 min. The collected fractions were combined and concentrated under vacuum. The residue was lyophilized to give (2S,3R)-2-((((9H-fluoren-9-yl) methoxy)carbonyl)amino)-3-(4-cyanophenyl)-3-hydroxypropanoic acid (A). MS ESI calculated for C₂₅H₂₁N₂O₅ [M+H]⁺ 429.14, found 429.25.

The slower peak was obtained at 9.21 min. The collected fractions were combined and concentrated under vacuum. The residue was lyophilized to give (2S,3S)-2-((((9H-fluoren-9-yl) methoxy)carbonyl)amino)-3-(4-cyanophenyl)-3-hydroxypropanoic acid (B). MS ESI calculated for C₂₅H₂₁N₂O₅ [M+H]⁺ 429.14, found 429.30.

Step 3: Ghaffar-Parkins's catalyst ([hydrogen bis(dimethylphosphinito-κP)]platinum (II), 0.023 g, 0.053 mmol) at RT was added to a stirred solution of (2S,3R)-2-((((9H-fluoren-9-yl) methoxy)carbonyl)amino)-3-(4-cyanophenyl)-3-hydroxypropanoic acid (A) (2.3 g, 5.3 mmol) in EtOH (40 mL) and water (20 mL). The resulting mixture was stirred at 80° C. for 2 h. The pH was adjusted to ˜3 with 1 N HCl. The solvent was concentrated under reduced pressure and the residue was purified by Rp-flash column chromatography with the following conditions: Column: Flash C18 (330 g); Mobile Phase A: water (0.05% TFA), Mobile Phase B: MeCN; Gradient elution: 2-45%; Detector: UV 210 nm. The fractions containing the product were concentrated under reduced pressure to afford (2S,3R)-2-((((9H-fluoren-9-yl)methoxy)carbonyl) amino)-3-(4-carbamoylphenyl)-3-hydroxypropanoic acid. MS ESI calculated for C₂₅H₂₃N₂O₆Na [M+Na]⁺ 469.15, found 469.10. ¹H NMR (400 MHz, DMSO-d₆): δ 12.84 (s, 1H), 8.01-7.78 (m, 5H), 7.63-7.61 (m, 2H), 7.54-7.20 (m, 8H), 5.84 (s, 1H), 5.27-5.23 (m, 1H), 4.34-4.32 (m, 1H), 4.19-4.00 (m, 3H).

Step 4: Ghaffar-Parkins's catalyst (0.011 g, 0.026 mmol) at RT was added to a stirred solution of (2S,3S)-2-((((9H-fluoren-9-yl)methoxy)carbonyl)amino)-3-(4-cyanophenyl)-3-hydroxypropanoic acid (B) (1.1 g, 2.6 mmol) in EtOH (22 mL) and water (11 mL). The resulting mixture was stirred at 80° C. for 2 h. The pH was adjusted to ˜3 with 1 N HCl. The solvent was concentrated under reduced pressure and the residue was purified by Rp-flash column chromatography with the following conditions: Column: Flash C18 (330 g); Mobile Phase A: water (0.05% TFA), Mobile Phase B: MeCN; Gradient elution: 2-45%; Detector: UV 210 nm. The fractions containing the product were concentrated under reduced pressure to afford (2S,3S)-2-((((9H-fluoren-9-yl)methoxy)carbonyl)amino)-3-(4-carbamoylphenyl)-3-hydroxypropanoic acid. MS ESI calculated for C₂₅H₂₂N₂O₆Na [M+Na]⁺ 469.15, found 469.10. ¹H NMR (400 MHz, DMSO-d₆): δ 12.80 (s, 1H), 8.01-7.78 (m, 5H), 7.75-7.54 (m, 3H), 7.54-7.23 (m, 7H), 5.87 (s, 1H), 4.85-4.82 (m, 1H), 4.23-4.04 (m, 4H).

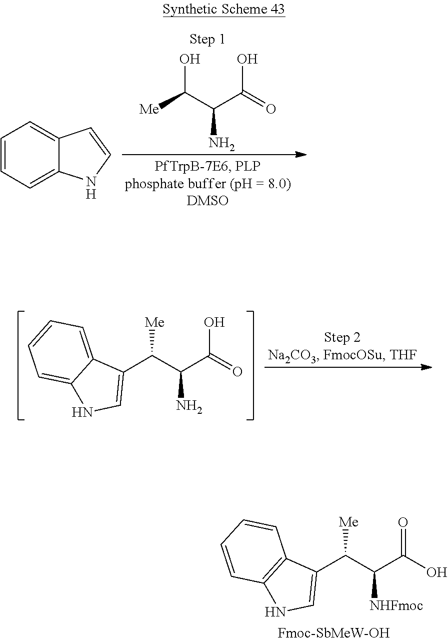

### (2S,3S)-2-((((9H-Fluoren-9-yl)methoxy)carbonyl)amino)-3-(1H-indol-3-yl)butanoic acid (Fmoc-SbMeW-OH)

Step 1: DMSO (20 mL, 4 V), indole (5.00 g, 42 mmol), (2S,3R)-2-amino-3-hydroxybutanoic acid (10.17 g, 85 mmol), 0.2 M potassium phosphate buffer (230 mL, 46 V, pH=8.0) were placed into a 500 mL 4-necked round-bottom flask purged and maintained with an inert atmosphere of N₂. The resulting solution was stirred at 65° C. PLP (46 mg, 0.18 mmol) in DI water (2 mL) and PfTrpB-7E6 (1.00 g, 20 wt. %) in 0.2 M potassium phosphate buffer (2 mL, pH=8) were added into the solution. The resulting solution was stirred for o.n. at 65° C. The reaction progress was monitored by LC-MS. Upon completion, the resulting solution was used directly in Step 2.

Step 2: Na₂CO₃ (9.05 g, 85.3 mmol), Fmoc-OSu (21.58 g, 64.0 mmol), and THF (100 mL) were added at 0° C. into the solution. The resulting solution was stirred for 3 h at RT. The solution was adjusted to pH=3 with aq. 6 N HCl. The mother liquor was collected by filtration and extracted with EtOAc (3×200 mL). The combined organic layers were washed with DI water (2×400 mL) and brine (400 mL), dried over anh. Na₂SO₄, filtered, and concentrated under vacuum. The crude product was triturated with DCM/n-heptane (1:1; v/v), and the solids were collected by filtration to afford (2S,3S)-2-((((9H-fluoren-9-yl)methoxy)carbonyl)amino)-3-(1H-indol-3-yl)butanoic acid. MS ESI calculated for C₂₇H₂₄N₂O₄[M+H]⁺ 441, found 441. ¹H NMR (400 MHz, DMSO-d₆): δ 10.88 (s, 1H), 7.87 (d, J=7.5 Hz, 2H), 7.69-7.56 (m, 3H), 7.39 (t, J=7.4 Hz, 2H), 7.38-7.21 (m, 4H), 7.18 (d, J=2.1 Hz, 1H), 7.05 (ddd, J=8.1, 6.9, 1.2 Hz, 1H), 6.96 (td, J=7.4, 7.0, 1.1 Hz, 1H), 4.43-4.32 (m, 1H), 4.21 (d, J=10.0 Hz, 1H), 4.16 (t, J=5.3 Hz, 2H), 3.50 (q, J=7.1 Hz, 1H), 1.33 (d, J=7.1 Hz, 3H).

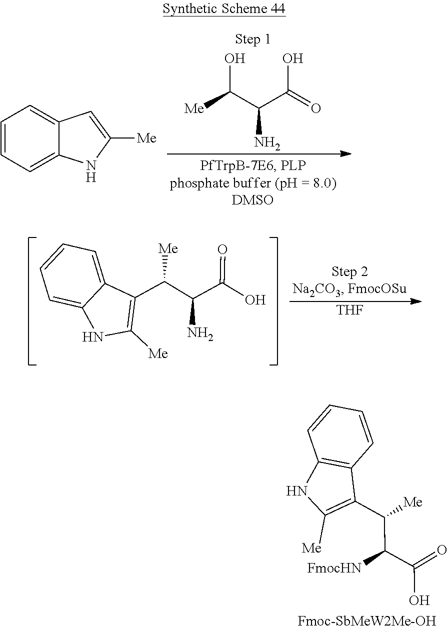

### (2S,3S)-2-((((9H-Fluoren-9-yl)methoxy)carbonyl)amino)-3-(2-methyl-1H-indol-3-yl)butanoic acid (Fmoc-SbMeW2Me-OH)

Step 1: 2-Methyl-1H-indole (100.00 g, 762.95 mmol), L-threonine (181.67 g, 1525.9 mmol), DMSO (400 mL), and potassium phosphate buffer (4.6 L, 0.2 M, pH=8) were placed into a 10-L 4-necked round-bottom flask, purged and maintained with an inert atmosphere of N₂. The reaction mixture was heated to 65° C. PfTrpB-7E6 (20.00 g, 20 wt. %) and PLP (2.02 g, 7.63 mmol) were then added. The reaction was stirred o.n. at 65° C. and then cooled to RT. This mixture was used directly in Step 2.

Step 2: Into the Step 1 reaction mixture, THF (2 L), Na₂CO₃ (160.68 g, 1516.0 mmol), and Fmoc-OSu (385.70 g, 1144.4 mmol) were added at 0° C. The reaction mixture was stirred o.n. at RT. The solids were filtered off. The resulting solution was extracted with EtOAc (3×2 L). The organic combined layers were washed with DI water (3×2 L) and brine (1.5 L), dried over anh. Na₂SO₄, filtered, and concentrated. The product was precipitated by the addition of DCM/n-heptane (2:1; v/v), and stirred o.n. at RT. The solids were collected by filtration to give (2S,3S)-2-((((9H-fluoren-9-yl)methoxy)carbonyl)amino)-3-(2-methyl-1H-indol-3-yl)butanoic acid. MS ESI calculated for C₂₈H₂₆N₂O₄ [M+H]⁺ 455, found 455. ¹H NMR (300 MHz, DMSO-d₆): δ 12.78 (s, 1H), 10.75 (s, 1H), 7.87 (d, J=7.6 Hz, 2H), 7.58 (d, J=7.4 Hz, 2H), 7.50 (d, J=7.8 Hz, 1H), 7.45-7.35 (m, 2H), 7.33-7.17 (m, 3H), 7.11-6.85 (m, 3H), 4.48 (t, J=8.5 Hz, 1H), 4.31-3.90 (m, 3H), 3.41-3.38 (m, 1H), 2.37 (d, J=1.4 Hz, 3H), 1.37 (dd, J=7.2, 1.6 Hz, 3H).

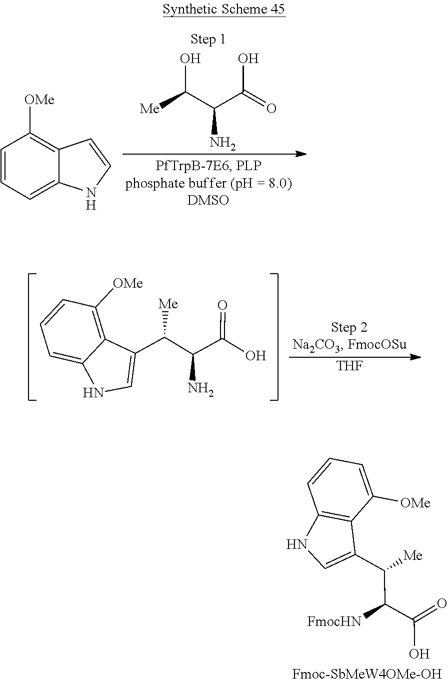

### (2S,3S)-2-((((9H-Fluoren-9-yl)methoxy)carbonyl)amino)-3-(4-methoxy-1H-indol-3-yl)butanoic acid (Fmoc-SbMeW40Me-OH)

Step 1: 4-Methoxy-1H-indole (100.00 g, 679.45 mmol), L-threonine (161.87 g, 1358.9 mmol), DMSO (400 mL) and potassium phosphate buffer (4.6 L, 0.2 M, pH=8) were placed into a 10 L 4-necked round-bottom flask, purged and maintained with an inert atmosphere of N₂. The reaction mixture was heated to 65° C. PfTrpB-7E6 (20.00 g, 20 wt. %) and PLP (1.24 g, 4.68 mmol) were then added. The resulting solution was stirred o.n. at 65° C. Upon completion, the mixture was cooled to RT and used directly in the next step.

Step 2: THF (2 L), Na₂CO₃ (144.00 g, 1358.6 mmol), and Fmoc-OSu (343.50 g, 1018 mmol) were added to the reaction mixture at 0° C. The resulting solution was stirred o.n. at RT. The solids were filtered off. The filtrate was extracted with EtOAc (3×2 L). The combined organic layers were washed with DI water (3×2 L) and brine (1.5 L), dried over anh. Na₂SO₄, filtered, and concentrated. The product was recrystallized with DCM/n-heptane (2:1; v/v). The solids were collected by filtration to give (2S,3S)-2-((((9H-fluoren-9-yl)methoxy)carbonyl) amino)-3-(4-methoxy-1H-indol-3-yl)butanoic acid. MS ESI calculated for C₂₈H₂₆N₂O₅ [M+H]⁺ 471, found 471. ¹H (400 MHz, DMSO-d₆): δ 12.48 (s, 1H), 10.89 (d, J=2.5 Hz, 1H), 7.87 (dt, J=7.7, 0.9 Hz, 2H), 7.60 (dd, J=12.7, 7.5 Hz, 2H), 7.40 (tdd, J=7.5, 2.5, 1.2 Hz, 3H), 7.34-7.26 (m, 1H), 7.26 (td, J=7.5, 6.9, 1.9 Hz, 1H), 7.06 (d, J=2.4 Hz, 1H), 7.05-6.94 (m, 2H), 6.50 (dd, J=7.2, 1.5 Hz, 1H), 4.31 (t, J=8.1 Hz, 1H), 4.25-3.98 (m, 3H), 3.84 (s, 3H), 3.66 (p, J=7.2 Hz, 1H), 1.33 (d, J=7.1 Hz, 3H).

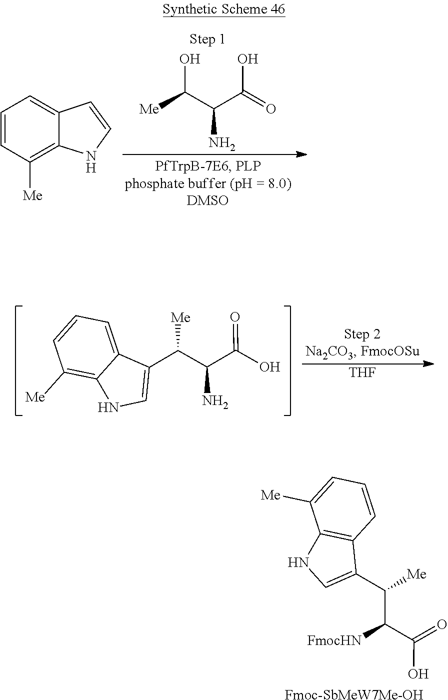

### (2S,3S)-2-((((9H-Fluoren-9-yl)methoxy)carbonyl)amino)-3-(7-methyl-1H-indol-3-yl)butanoic acid (Fmoc-SbMeW7Me-OH)

Step 1: 7-Methyl-1H-indole (200.0 g, 1525 mmol), DMSO (800.0 mL), 0.2 M potassium phosphate buffer (9200.0 mL, pH=8), PLP (1.94 g, 7.3 mmol), L-threonine (363.2 g, 3049.3 mmol) and PfTrpB-7E6 (40.0 g, 20 wt. %) at RT were placed into a 20 L 4-necked round-bottom flask. The resulting solution was stirred for o.n. at 65° C. under N₂. Upon completion, the resulting solution was used directly in Step 2.

Step 2: Na₂CO₃ (323.18 g, 3049 mmol), Fmoc-OSu (1028.59 g, 3049 mmol), and THF (2.0 L) were added to the reaction mixture of Step 1 at <15° C. The resulting solution was stirred for 3 h at RT under N₂. The mixture was acidified to pH ˜3 with an aq. 6 N HCl solution. The mother liquor was collected after filtration and extracted with EtOAc (3×10 L). The combined organic layers were washed with DI water (2×5 L) and brine (2 L), dried over anh. Na₂SO₄, filtered, and concentrated under vacuum. The residue was triturated with DCM/hexanes (10 V:5 V) o.n. and filtered to provide (2S,3S)-2-((((9H-fluoren-9-yl)methoxy)carbonyl)amino)-3-(7-methyl-1H-indol-3-yl)butanoic acid. MS ESI calculated for C₂₈H₂₆N₂O₄ [M+H]⁺ 455, found 455. ¹NMR (400 MHz, DMSO-d₆): δ 12.67 (s, 1H), 10.85 (d, J=2.5 Hz, 1H), 7.89 (d, J=7.5 Hz, 2H), 7.66 (d, J=7.5 Hz, 2H), 7.50-7.36 (m, 3H), 7.29 (td, J=7.7, 3.7 Hz, 3H), 7.21 (d, J=2.5 Hz, 1H), 6.96-6.82 (m, 2H), 4.41 (dd, J=8.5, 7.1 Hz, 1H), 4.28-4.12 (m, 3H), 3.52 (q, J=7.1 Hz, 1H), 2.45 (s, 3H), 1.36 (d, J=7.1 Hz, 3H).

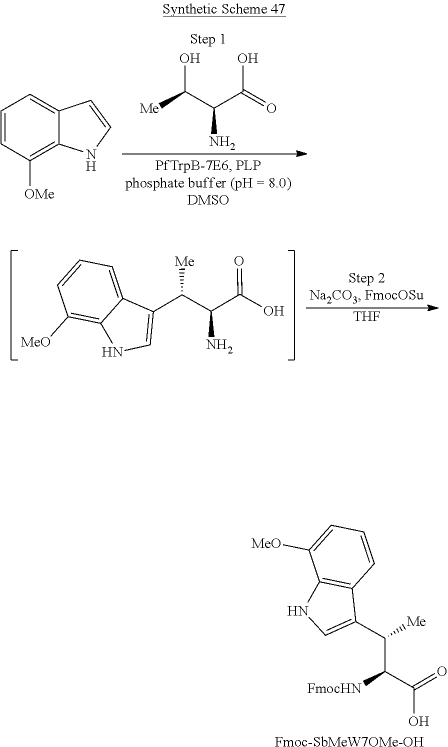

### (2S,3S)-2-((((9H-Fluoren-9-yl)methoxy)carbonyl)amino)-3-(7-methoxy-1H-indol-3-yl)butanoic acid (Fmoc-SbMeW70Me-OH)

Step 1: DMSO (640 mL, 4V), 7-methoxy-1H-indole (50 g, 0.34 mol), (2S,3R)-2-amino-3-hydroxybutanoic acid (81 g, 0.68 mol), and 0.2 M potassium phosphate buffer (2300 mL, pH=8.0, 46V) were placed into a 5 L 4-necked round-bottom flask, purged and maintained with an inert atmosphere of N₂. The resulting solution was heated to 65° C. PLP (5 g, 0.02 mol) and PfTrpB-7E6 (50 g, 100 wt. %) were added into the solution. The reaction mixture was stirred o.n. at 65° C. Upon completion, the reaction mixture was used directly in Step 2.

Step 2: Na₂CO₃ (72 g, 0.68 mol), Fmoc-OSu (230 g, 0.68 mol), and THF (500 mL) were placed into the Step 1 reaction mixture at 0° C. The reaction was stirred for 3 h at RT. The solution was then adjusted to pH ˜5-6 with an aq. 6 N HCl solution. The mixture was then filtered, and the filtrate was extracted with EtOAc (3×1 L). The combined organic layers were washed with DI water (2×1 L) and brine (1 L), dried over anh. Na₂SO₄, filtered, and concentrated under vacuum. The residue was purified by trituration with DCM/heptane (1:1; v/v) for 1 h to yield (2S,3S)-2-((((9H-fluoren-9-yl)methoxy)carbonyl)amino)-3-(7-methoxy-1H-indol-3-yl)butanoic acid. MS ESI calculated for C₂₈H₂₆N₂O₅ [M+H]⁺ 471, found 471. ¹H NMR (400 MHz, DMSO-d₆): δ 12.63 (s, 1H), 10.95 (d, J=2.6 Hz, 1H), 7.87 (dt, J=7.6, 1.0 Hz, 2H), 7.68-7.61 (m, 2H), 7.40 (tt, J=7.5, 1.1 Hz, 2H), 7.28 (tdd, J=7.5, 3.4, 1.1 Hz, 3H), 7.20 (d, J=8.0 Hz, 1H), 7.10 (d, J=2.5 Hz, 1H), 6.89 (t, J=7.8 Hz, 1H), 6.66-6.59 (m, 1H), 4.36 (dd, J=8.5, 7.2 Hz, 1H), 4.27-4.07 (m, 3H), 3.89 (s, 3H), 3.44 (q, J=7.1 Hz, 1H), 1.33 (d, J=7.1 Hz, 3H).

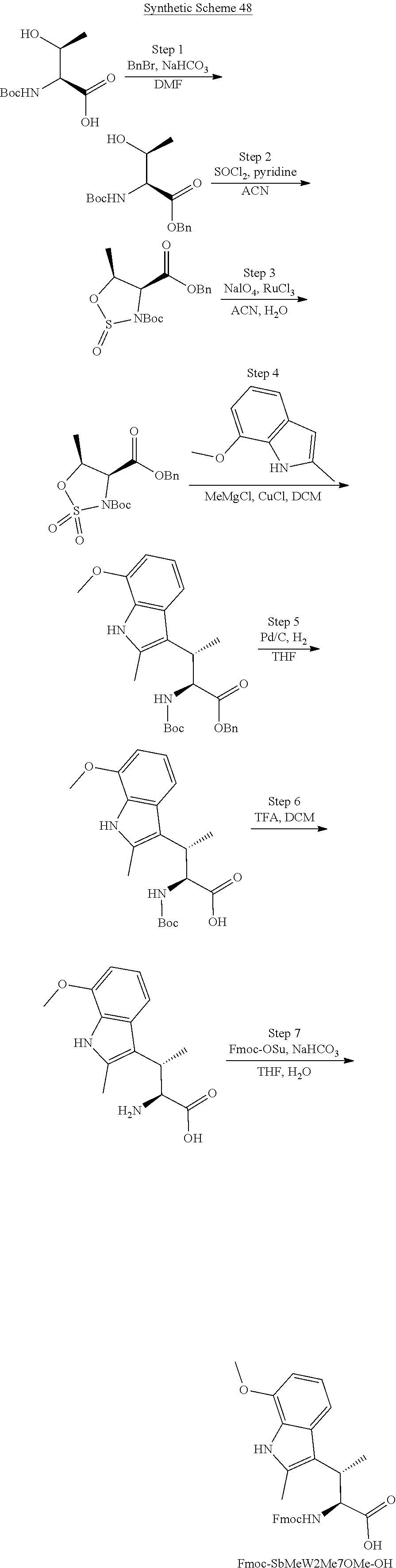

### (2S,3S)-2-((((9H-Fluoren-9-yl)methoxy)carbonyl)amino)-3-(7-methoxy-2-methyl-1H-indol-3-yl)butanoic acid (Fmoc-SbMeW2Me7OMe-OH)

Step 1: NaHCO₃ (5.8 g, 68.4 mmol) and benzyl bromide (4.7 g, 27.4 mmol) were added to a solution of (tert-butoxycarbonyl)-L-allo-threonine (5.0 g, 22.8 mmol) in DMF (100 mL) at 0° C. under Ar atmosphere. The resulting mixture was stirred o.n. at RT. The pH of the solution was adjusted to ˜3 by addition of 1 N HCl then extracted with EtOAc (600 mL). The organic layer was washed with brine (3×200 mL), dried over anh. Na₂SO₄, and filtered. The filtrate was concentrated under reduced pressure and the residue was purified by silica gel column chromatography, eluting with EtOAc 0-30% in PE to give benzyl (tert-butoxycarbonyl)-L-allo-threoninate. MS ESI calculated for C₁₆H₂₃NO₅Na [M+Na]⁺ 332.16, found 332.00. ¹H NMR (300 MHz, chloroform-d): δ 7.39-3.37 (m, 1H), 7.37-7.30 (m, 4H), 5.44 (s, 1H), 5.26-5.14 (m, 2H), 4.43 (s, 1H), 4.23-4.09 (m, 1H), 1.44 (s, 9H), 1.14 (d, J=6.5 Hz, 3H).

Step 2: SOCl₂ (6.6 g, 52.5 mmol) and pyridine (8.3 g, 105 mmol) at −40° C. were added to a solution of benzyl (tert-butoxycarbonyl)-L-allo-threoninate (6.5 g, 21.0 mmol) in MeCN (120 mL). The resulting mixture was stirred for 30 min at −40° C., then for 1 h at RT. The reaction was concentrated under vacuum. The residue was purified by silica gel column chromatography, eluting with EtOAc in PE (1:3; v/v) to afford 4-benzyl 3-(tert-butyl) (4S,5S)-5-methyl-1,2,3-oxathiazolidine-3,4-dicarboxylate 2-oxide. ¹H NMR (300 MHz, chloroform-d): δ 7.36-7.26 (m, 5H), 5.43-5.28 (m, 1H), 5.27-4.99 (m, 2H), 4.14 (d, J=9.1 Hz, 1H), 1.68-1.19 (m, 12H).

Step 3: Ruthenium (III) chloride (0.077 g, 0.371 mmol) and NaIO₄ (4.77 g, 22.28 mmol) were added to a solution of 4-benzyl 3-(tert-butyl) (4S,5S)-5-methyl-1,2,3-oxathiazolidine-3,4-dicarboxylate 2-oxide (6.6 g, 18.6 mmol) in MeCN (75 mL) and water (75 mL) at 0° C. The resulting mixture was stirred for 18 h at RT. The reaction mixture was then diluted with water (200 mL) and extracted with EtOAc (3×400 mL). The combined organic layers were washed with brine (3×200 mL), dried over anh. Na₂SO₄, and filtered. The filtrate was concentrated under vacuum and the crude oil was purified by silica gel column chromatography, eluting with EtOAc in PE (1:2; v/v) to afford 4-benzyl 3-(tert-butyl) (4S,5S)-5-methyl-1,2,3-oxathiazolidine-3,4-dicarboxylate 2,2-dioxide. ¹H NMR (300 MHz, chloroform-d): δ 7.38-7.37 (m, 5H), 5.42-5.16 (m, 2H), 5.10-5.06 (m, 1H), 4.70-4.65 (m, 1H), 1.55-1.49 (s, 9H), 1.41-1.39 (m, 3H).

Step 4: Methylmagnesium chloride (3.0 M in THF, 5.13 mL, 15.40 mmol at 0° C. under Ar atmosphere) were added dropwise over a 30 min period to a solution of 7-methoxy-2-methyl-1H-indole (2.86 g, 17.77 mmol) in DCM (100 mL). Copper(I) iodide (1.525 g, 15.40 mmol) and 4-benzyl 3-(tert-butyl) (4S,5S)-5-methyl-1,2,3-oxathiazolidine-3,4-dicarboxylate 2,2-dioxide (4.4 g, 11.85 mmol) were then added. The resulting mixture was stirred for 2 h at 0° C., then o.n. at RT. The mixture was concentrated under reduced pressure. The residue was purified by Rp-flash column chromatography with the following conditions: Column: Flash C18 (330 g); Mobile Phase A: water (0.05% TFA), Mobile Phase B: MeCN; Gradient Elution: 2-55%; Detector: UV 210 nm. The fractions containing the product were concentrated under reduced pressure to afford benzyl (2S,3S)-2-((tert-butoxycarbonyl)amino)-3-(7-methoxy-2-methyl-1H-indol-3-yl)butanoate. MS ESI calculated for C₂₆H₃₂N₂O₅Na [M+Na]⁺ 475.23, found 475.30.

Step 5: Benzyl (2S,3S)-2-((tert-butoxycarbonyl)amino)-3-(7-methoxy-2-methyl-1H-indol-3-yl)butanoate (2.1 g, 4.6 mmol) was dissolved in THF (60 mL). The flask was evacuated and refilled with N₂ (5 times). Pd/C (400 mg, 0.38 mmol, dry, 10 wt. %) was added into the flask. The flask was evacuated and backfilled with H₂ (5 times). The resulting mixture was stirred for 4 h at RT under an atmosphere of dihydrogen. The flask was evacuated and refilled with N₂ (5 times). The resulting mixture was filtered through a Celite® pad. The filtered cake was washed with MeOH (3×100 mL). The filtrate was concentrated under reduced pressure. The residue was purified by silica gel column chromatography, eluting with MeOH in DCM (1:10; v/v) to afford (2S,3S)-2-((tert-butoxycarbonyl)amino)-3-(7-methoxy-2-methyl-1H-indol-3-yl)butanoic acid. MS ESI calculated for C₁₉H₂₆N₂O₅Na [M+Na]⁺ 385.18, found 385.25.

Step 6: TFA (7 mL) was added to a stirred solution of (2S,3S)-2-((tert-butoxycarbonyl) amino)-3-(7-methoxy-2-methyl-1H-indol-3-yl)butanoic acid (1.17 g, 3.23 mmol) in DCM (21 mL) at RT. The resulting mixture was stirred at RT for 2.5 h. The solvent was evaporated under reduced pressure to give crude (2S,3S)-2-amino-3-(7-methoxy-2-methyl-1H-indol-3-yl)butanoic acid, which was used in Step 7 without further purification. MS ESI calculated for Chemical Formula: C₁₉H₂₇N₂O₅ [M+H]⁺ 263.13, found 263.15.

Step 7: NaHCO₃(1.3 g, 15.3 mmol) and Fmoc-OSu (0.9 g, 2.7 mmol) were added at RT to a stirred solution of (2S,3S)-2-amino-3-(7-methoxy-2-methyl-1H-indol-3-yl)butanoic acid (0.8 g, 3.1 mmol) in THF (12 mL) and water (12 mL). The resulting mixture was stirred o.n. at RT. The pH was adjusted to ˜3 by addition of 1 N HCl. The mixture was extracted with EtOAc (3×100 mL). The organic phase was washed with brine (3×100 mL), dried over anh. Na₂SO₄, filtered, and the solvent was evaporated under reduced pressure. The residue was purified by Rp-flash column chromatography with the following conditions: Column: Flash C18 (330 g); Mobile Phase A: water (0.05% TFA), Mobile Phase B: MeCN; Gradient Elution: 25-70%; Detector: UV 210 nm. The fractions containing the product were concentrated under reduced pressure to afford (2S,3S)-2-((((9H-fluoren-9-yl)methoxy)carbonyl)amino)-3-(7-methoxy-2-methyl-1H-indol-3-yl)butanoic acid. MS ESI calculated for C₂₉H₂₉N₂O₅[M+H]⁺ 485.20, found 485.10. ¹H NMR (400 MHz, DMSO-d₆): δ 12.80 (s, 1H), 10.81 (s, 1H), 7.87-7.85 (m, 2H), 7.58-7.56 (m, 2H), 7.41-7.39 (m, 2H), 7.27-7.26 (m, 2H), 7.11 (d, J=8.0 Hz, 1H), 6.96 (d, J=9.0 Hz, 1H), 6.85-6.83 (m, 1H), 6.56 (d, J=7.7 Hz, 1H), 4.48-4.46 (m, 1H), 4.23-4.15 (m, 1H), 4.12-4.10 (m, 2H), 3.87 (s, 3H), 3.38-3.36 (m, 1H), 2.36 (s, 3H), 1.36 (d, J=7.2 Hz, 3H).

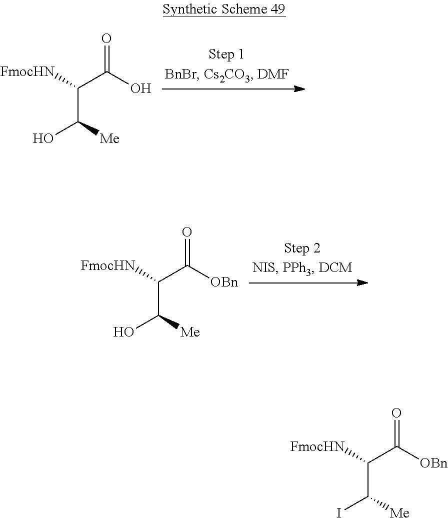

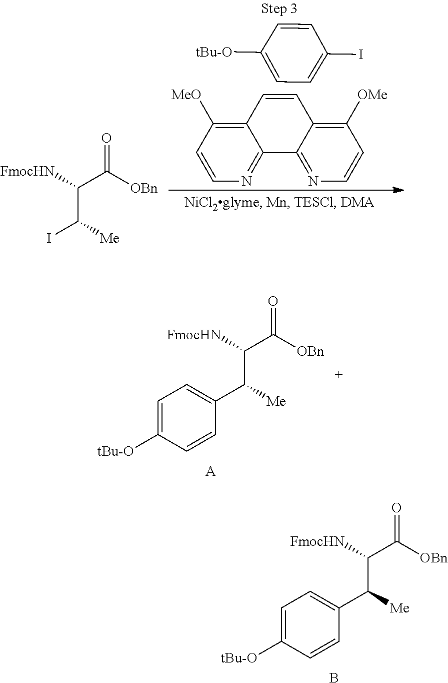

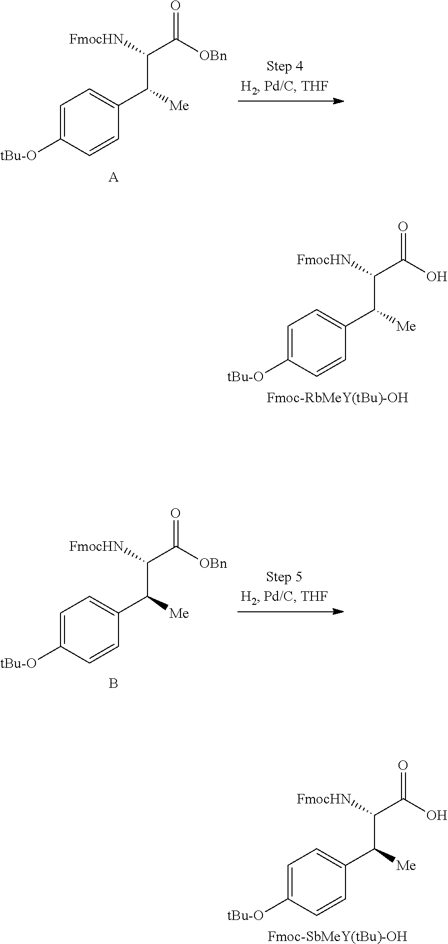

### (2S,3R)-2-((((9H-Fluoren-9-yl)methoxy)carbonyl)amino)-3-(4-(tert-butoxy)phenyl)butanoic acid (Fmoc-RbMeY(tBu)-OH) and (2S,3S)-2-((((9H-fluoren-9-yl)methoxy)carbonyl)amino)-3-(4-(tert-butoxy)phenyl)butanoic acid (Fmoc-SbMeY(tBu)-OH)

Step 1: BnBr (501 g, 2.93 mol) was added dropwise to a solution of (((9H-fluoren-9-yl) methoxy)carbonyl)-L-threonine (500 g, 1.46 mol) in DMF (3.00 L) at RT. Cs₂CO₃ (954 g, 2.93 mol) was added into the solution and the resulting suspension was stirred at RT for 3 h. The reaction mixture was poured into DI water (20 L) at RT. The solids were recovered by filtration. The filtered cake was rinsed with DI water (2×1 L) and dried under vacuum to give the crude product. The crude solids were triturated with MTBE (10 L) at RT for 2 h. The suspension was filtered, and the filtered cake dried under reduced pressure. The residue was triturated with PE (10 L) at RT for 6 h. The suspension was filtered and the filtered cake dried under reduced pressure to give benzyl (((9H-fluoren-9-yl)methoxy)carbonyl)-L-threoninate. ¹H NMR (400 MHz, methanol-d₄): δ 7.79 (d, J=7.6 Hz, 2H), 7.66-7.65 (m, 2H), 7.40-7.27 (m, 10H), 5.19 (s, 2H), 4.42-4.19 (m, 5H), 1.18 (d, J=6.4 Hz, 3H).

Step 2: N-iodosuccinimide (547 g, 2.43 mol) and PPh₃ (638 g, 2.43 mol) at 0° C. were added to a solution of benzyl (((9H-fluoren-9-yl)methoxy)carbonyl)-L-threoninate (350 g, 811 mmol) in DCM (3.5 L). The solution was stirred at 30° C. for 5 h. The reaction mixture was concentrated under reduced pressure. The resulting residue was purified by silica gel column chromatography (PE/DCM/EtOAc=10:10:1; v/v/v) to give benzyl (2R,3S)-2-((((9H-fluoren-9-yl)methoxy)carbonyl)amino)-3-iodobutanoate. ¹H NMR (400 MHz, methanol-d₄): δ 7.79 (d, J=7.6 Hz, 2H), 7.68 (d, J=7.2 Hz, 2H), 7.40-7.27 (m, 10H), 5.20 (q, J=9.6 Hz, 2H), 4.53-4.50 (m, 2H), 4.42-4.37 (m, 2H), 4.24-4.21 (m, 1H), 1.84-1.79 (m, 3H).

Step 3: Solution A: NiCl₂·glyme (4.06 g, 18.5 mmol) and 4,7-dimethoxy-1,10-phenanthroline (4.44 g, 18.5 mmol) were dissolved into anh. DMA (500 mL) under N₂ and the solution was stirred at RT for 30 min. Solution B: Benzyl (2R,3S)-2-((((9H-fluoren-9-yl) methoxy)carbonyl)amino)-3-iodobutanoate (100 g, 185 mmol) and 1-(tert-butoxy)-4-iodobenzene (51.0 g, 185 mmol) were dissolved into anh. DMA (250 mL). Solution B was added to solution A under N₂. A suspension of manganese (20.3 g, 369 mmol) in anh. DMA (20.1 mL) was added to the reaction mixture under N₂. Triethylsilyl chloride (13.9 g, 92.4 mmol, 15.7 mL) was added to the suspension under N₂. The suspension was stirred at RT for 12 h under N₂. The solids were removed by filtration and rinsed with EtOAc (3×800 mL). The filtrate was poured into DI water (2.0 L) and extracted with EtOAc (3×1.5 L). The combined organic layers were washed with brine (2×800 mL), dried over anh. Na₂SO₄, filtered, and concentrated under reduced pressure. The residue was purified by silica gel column chromatography, eluting with EtOAc in PE (50:1-0:1; v/v) to afford benzyl (2S,3R)-2-((((9H-fluoren-9-yl)methoxy)carbonyl) amino)-3-(4-(tert-butoxy)phenyl)butanoate A and benzyl (2S,3S)-2-((((9H-fluoren-9-yl)methoxy) carbonyl)amino)-3-(4-(tert-butoxy)phenyl)butanoate B as a mixture. The diastereoisomeric mixture was separated by silica gel column chromatography, eluting with EtOAc in PE (100:1-0:1; v/v).

Benzyl (2S,3R)-2-((((9H-fluoren-9-yl)methoxy)carbonyl)amino)-3-(4-(tert-butoxy)phenyl) butanoate (A). ¹H NMR (400 MHz, DMSO-d₆): δ 7.77 (d, J=7.6 Hz, 2H), 7.58 (d, J=7.2 Hz, 2H), 7.42-7.31 (m, 7H), 7.26-7.24 (m, 2H), 7.10-6.99 (m, 2H), 6.87 (d, J=8.4 Hz, 2H), 5.32-5.28 (m, 1H), 5.01 (dd, J=40.4, 12.4 Hz, 2H), 4.62-4.58 (m, 1H), 4.44-4.41 (m, 1H), 4.39-4.34 (m, 1H), 4.23-4.18 (m, 1H), 3.22-3.17 (m, 1H), 1.34-1.28 (m, 12H).

Benzyl (2S,3S)-2-((((9H-fluoren-9-yl)methoxy)carbonyl)amino)-3-(4-(tert-butoxy)phenyl) butanoate (B). ¹H NMR (400 MHz, chloroform-d): δ 7.77 (d, J=7.6 Hz, 2H), 7.56 (d, J=7.2 Hz, 2H), 7.41-7.28 (m, 9H), 6.90 (dd, J=26.0, 8.4 Hz, 4H), 5.20-5.04 (m, 3H), 4.64-4.61 (m, 1H), 4.42-4.35 (m, 2H), 4.22-4.20 (m, 1H), 3.40-3.36 (m, 1H), 1.33-1.27 (m, 12H).

Step 4: Pd/C (5 g, 10 wt. %) was added under Ar to a flask containing a solution of benzyl (2S,3R)-2-((((9H-fluoren-9-yl)methoxy)carbonyl)amino)-3-(4-(tert-butoxy)phenyl) butanoate (A) (60.0 g, 106 mmol) in THF (400 mL). The flask was evacuated and backfilled with H₂ (3 times). The resulting mixture was stirred for 3 h at RT under an atmosphere of dihydrogen (15 psi). The flask was evacuated and refilled with N₂ (5 times). The suspension was filtered through a pad of Celite®. The filtered cake was rinsed with THF (1.0 L). The filtrate was concentrated under reduced pressure. The crude product was triturated with PE (500 mL) for 4 h. The resulting suspension was filtered, and the filtered cake was rinsed with PE (200 mL). The filtered cake was dissolved in MeCN (250 mL) and the resulting solution was poured into DI water (1.5 L) with stirring. The resulting suspension was filtered and the filtered cake rinsed with DI water (200 mL) and dried under vacuum to give (2S,3R)-2-((((9H-fluoren-9-yl)methoxy) carbonyl)amino)-3-(4-(tert-butoxy)phenyl)butanoic acid. MS ESI calculated for C₂₉H₃₁NO₅ [M+Na] 496, found 496. ¹H NMR (400 MHz, DMSO-d₆): δ 12.23 (br. s, 1H), 7.86 (d, J=7.6 Hz, 2H), 7.38-7.30 (m, 2H), 7.41 (t, J=7.6 Hz, 2H), 7.19-7.14 (m, 2H), 7.15 (d, J=8.4 Hz, 2H), 6.84 (d, J=8.4 Hz, 2H), 4.15-4.06 (m, 4H), 3.16-3.18 (m, 1H), 1.25 (s, 12H).

Step 5: Pd/C (8 g, 10 wt. %) was added under Ar to a flask containing a solution of benzyl (2S,3S)-2-((((9H-fluoren-9-yl)methoxy)carbonyl)amino)-3-(4-(tert-butoxy)phenyl) butanoate (B) (70.0 g, 124 mmol) in THF (450 mL). The flask was evacuated and backfilled with H₂ (3 times). The resulting mixture was stirred for 3 h at RT under an atmosphere of dihydrogen (15 psi). The flask was evacuated and refilled with N₂ (5 times). The suspension was filtered through a pad of Celite®. The filtered cake was rinsed with THF (1.0 L). The filtrate was concentrated under reduced pressure. The crude product was triturated with a mixture of PE and MTBE (10:1, 500 mL) for 4 h. The resulting suspension was filtered and the filtered cake was rinsed with PE (200 mL) and dried under vacuum to give (2S,3S)-2-((((9H-fluoren-9-yl) methoxy)carbonyl)amino)-3-(4-(tert-butoxy)phenyl)butanoic acid. MS ESI calculated for C₂₉H₃₁NO₅ [M+Na] 496, found 496. ¹H NMR (400 MHz, DMSO-d₆): δ 7.85 (d, J=7.6 Hz, 2H), 7.61 (d, J=8.0 Hz, 2H), 7.40 (t, J=7.6 Hz, 2H), 7.32 (t, J=7.2 Hz, 2H), 7.16 (d, J=8.4 Hz, 2H), 6.97 (br. s, 1H), 6.84 (d, J=8.4 Hz, 2H), 4.25-4.07 (m, 4H), 3.17-3.10 (m, 1H), 1.24 (s, 12H).

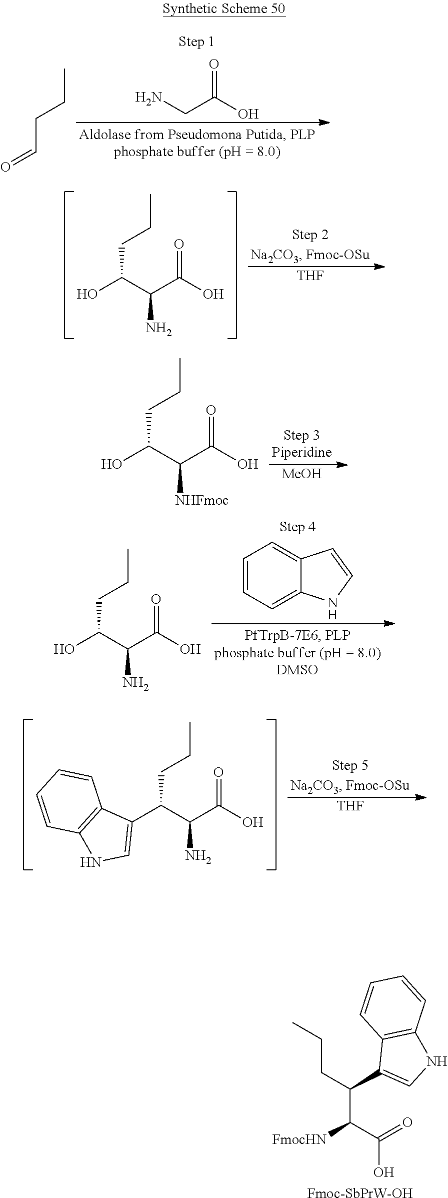

### (2S,3S)-2-((((9H-Fluoren-9-yl)methoxy)carbonyl)amino)-3-(1H-indol-3-yl)hexanoic acid(Fmoc-SbPrW-OH)

Step 1: Into a 5-L four-neck round-bottom flask purged and maintained under nitrogen atmosphere was placed glycine (104.2 g, 1.39 mol), aq. 1 M potassium phosphate buffer (2.5 L, 50V, pH=8.0), and PLP (736.1 mg, 0.003 mol). Aldolase from Pseudomona Putida (25.0 g, 50 wt. %) was added into the solution at RT. Butyraldehyde (50 g, 0.69 mol) was added, and then the resulting mixture was stirred o.n. at RT to provide (2S,3R)-2-amino-3-hydroxyhexanoic acid. MS ESI calculated for C₆H₁₃NO₃ [M+H]⁺ 148, found 148. The reaction mixture was used directly in the next step.

Step 2: THF (500 mL), Na₂CO₃ (147.2 g, 1.39 mol), and Fmoc-OSu (468.1 g, 1.39 mol) were added to the reaction mixture at 0° C. The mixture was stirred for 3 h at RT. The mixture was adjusted to pH=3 with aq. 6 N HCl. The mixture was then filtered, and the filtrate was extracted with EtOAc (3×2 L). The organic layers were combined, dried over anh. Na₂SO₄, and concentrated under vacuum. The residue was purified via silica gel column with MeOH in DCM (1:15; v/v) to afford (2S,3R)-2-(((9H-fluoren-9-yl)methoxy)carbonylamino)-3-hydroxyhexanoic acid. MS ESI calculated for C₂₁H₂₃NO₅ [M+H]⁺ 370, found 370.

Step 3: Into a 1-L three-neck round-bottom flask purged and maintained with an inert atmosphere of nitrogen was placed (2S,3R)-2-(((9H-fluoren-9-yl)methoxy)carbonylamino)-3-hydroxyhexanoic acid (42 g, 0.11 mol) and MeOH (820 mL) at RT. Piperidine (48.5 g, 0.569 mol) was added and the resulting solution was stirred o.n. at RT. The reaction was then quenched by the addition of water (1 L). The solution was extracted with EtOAc/PE (2:1; v/v; 3×300 mL). The water layers were combined and concentrated under vacuum. The crude product was slurried with n-heptane (420 mL) and the solids were collected by filtration to afford crude (2S,3R)-2-amino-3-hydroxyhexanoic acid. MS ESI calculated for C₆H₁₃NO₃ [M+H]⁺ 148, found 148.

Step 4: Into a 1-L three-neck round-bottom flask purged and maintained with an inert atmosphere of nitrogen were added (2S,3R)-2-amino-3-hydroxyhexanoic acid crude (9.36 g, 0.064 mol), 0.1 M potassium phosphate buffer (468 mL, pH=8.0), DMSO (46.8 mL), and 1H-indole (7.45 g, 0.064 mol). The resulting solution was stirred at 65° C. PLP (675 mg, 0.003 mol) and PfTrpB-7E6 (18.72 g, 200 wt. %) were added to the solution. The reaction mixture was stirred o.n. at 65° C. to afford (2S,3S)-2-amino-3-(1H-indol-3-yl)hexanoic acid, which was used directly in the next step. MS ESI calculated for C₁₄H₁₈N₂O₂ [M+H]⁺ 247, found 247.

Step 5: Na₂CO₃ (27 g, 0.254 mol), Fmoc-OSu (85.82 g, 0.254 mol), and THF (187.2 mL) were added to the reaction mixture at 0° C. The reaction was stirred for o.n. at RT. The solution was then adjusted to pH=3 with aq. 6 N HCl. The mixture was then filtered, and the filtrate was extracted with EtOAc (3×500 mL). The organic layers were combined, dried over anh. Na₂SO₄, and concentrated under vacuum. The residue was purified on a silica gel column, eluting with MeOH in DCM (1:15; v/v) to provide (2S,3S)-2-((((9H-fluoren-9-yl)methoxy)carbonyl)amino)-3-(1H-indol-3-yl)hexanoic acid. MS ESI calculated for C₂₉H₂₈N₂O₄ [M+H]⁺ 469, found 469. ¹H NMR (400 MHz, DMSO-d₆): δ 10.92 (s, 1H), 7.89 (d, J=7.5 Hz, 2H), 7.64 (d, J=7.5 Hz, 2H), 7.70-7.55 (m, 3H), 7.45-7.24 (m, 6H), 7.15-6.95 (m, 3H), 4.45-4.37 (m, 1H), 4.31-4.13 (m, 3H), 3.55-3.45 (m, 1H), 1.80-1.62 (m, 2H), 0.82 (t, J=7.4 Hz, 3H).

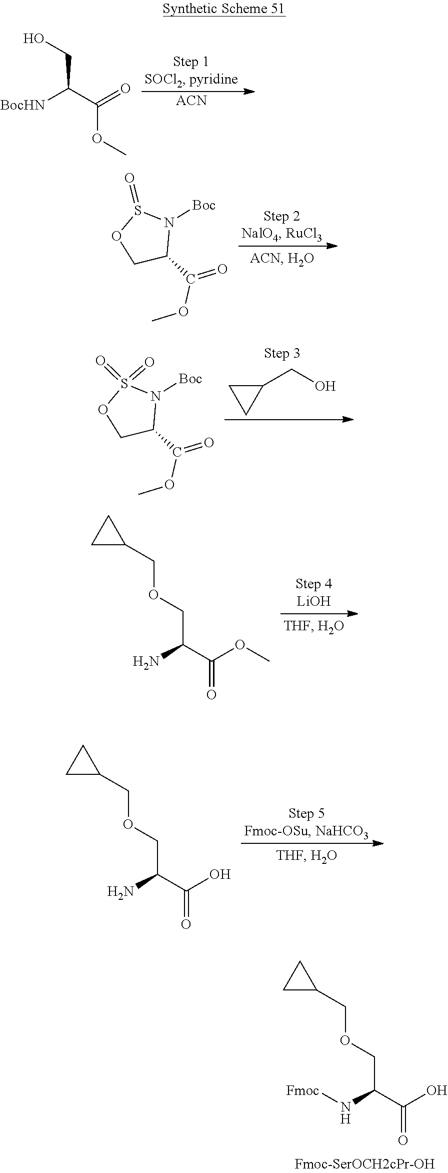

### N-(((9H-Fluoren-9-yl)methoxy)carbonyl)-O-(cyclopropylmethyl)-L-serine (Fmoc-SerOCH2cPr-OH)

Step 1: Pyridine (36.1 g, 456 mmol) and thionyl chloride (27.1 g, 228 mmol) were added to a solution of methyl (tert-butoxycarbonyl)-L-serinate (20.0 g, 91 mmol) in acetonitrile (400 mL) at −40° C. and under Ar. After stirring at −40° C. for 20 min, the cold bath was removed, and the reaction was stirred at RT for 30 min. The solution was concentrated under reduced pressure and the residue was purified by silica gel column chromatography, eluting with EtOAc 0-50% in PE to afford 3-(tert-butyl) 4-methyl (4S)-1,2,3-oxathiazolidine-3,4-dicarboxylate 2-oxide. ¹H NMR (300 MHz, chloroform-d): δ 5.13-4.87 (m, 2H), 4.71-4.47 (m, 1H), 3.83 (s, 3H), 1.53 (s, 9H).

Step 2: Sodium periodate (13.6 g, 63.3 mmol) and ruthenium(III) chloride (220 mg, 1.1 mmol) at 0° C. were added to a stirred solution of 3-(tert-butyl) 4-methyl (4S)-1,2,3-oxathiazolidine-3,4-dicarboxylate 2-oxide (14.0 g, 52.8 mmol) in acetonitrile (140 mL). Water (140 mL) was added to the mixture at 0° C. and stirred o.n. at RT. The reaction mixture was extracted with EtOAc (3×500 mL). The combined organic layers were washed with brine (3×200 mL), dried over anh. Na₂SO₄, and filtered. The filtrate was concentrated under reduced pressure and the residue was purified by silica gel column chromatography, eluting with EtOAc 0-50% in PE to afford 3-(tert-butyl) 4-methyl (S)-1,2,3-oxathiazolidine-3,4-dicarboxylate 2,2-dioxide. ¹H NMR (300 MHz, chloroform-d): δ 4.88-4.61 (m, 3H), 3.87 (s, 3H), 1.56 (s, 9H).

Step 3: A mixture of 3-(tert-butyl) 4-methyl (S)-1,2,3-oxathiazolidine-3,4-dicarboxylate 2,2-dioxide (7.1 g, 25.2 mmol) and cyclopropylmethanol (1.8 g, 25.2 mmol) was stirred at 90° C. o.n. The reaction mixture was cooled to RT and concentrated under reduced pressure to give methyl O-(cyclopropylmethyl)-L-serinate, which was used directly in Step 4 without purification.

Step 4: An aqu.1 N LiOH solution (50.5 mL, 50.5 mmol) at 0° C. was added to a stirred solution of methyl O-(cyclopropylmethyl)-L-serinate (4.37 g, 25.2 mmol) in THF (40 mL). The resulting solution was stirred at RT for 4 h. The reaction was acidified with 1 M HCl (50 mL) to pH ˜2-3 and extracted with EtOAc (3×500 mL). The combined organic layer was washed with brine (3×100 mL), dried over anh. Na₂SO₄, and filtered. The filtrate was concentrated under reduced pressure to afford O-(cyclopropylmethyl)-L-serine, which was used directly in Step 5.

Step 5: NaHCO₃(10.6 g, 126 mmol) and Fmoc-OSu (7.6 g, 22.6 mmol) were added to a stirred solution of O-(cyclopropylmethyl)-L-serine (4.0 g, 25.1 mmol, crude) in THF (40 mL) and water (40 mL) at 0° C. The mixture was stirred o.n. at RT. The pH value was adjusted to ˜5 with 1 M HCl and extracted with EtOAc (2×200 mL). The combined organic layers were washed with brine (200 mL), dried over anh. Na₂SO₄, and filtered. The filtrate was concentrated under reduced pressure and the residue was purified by Rp Flash (Column: Flash C18 330 g; Mobile Phase A: water (0.05% NH₄HCO₃), Mobile Phase B: MeCN; Flow rate: 70 mL/min; Gradient: 2-20% B in 5 min; 20-30% B in 5 min; 30-80% B in 25 min, Detector: UV 210 nm) to afford N-(((9H-fluoren-9-yl)methoxy)carbonyl)-O-(cyclopropylmethyl)-L-serine. MS ESI calculated for C₂₂H₂₄NO₅ [M+H]⁺ 382.16, found 382.20. ¹H NMR (300 MHz, methanol-d₄): δ 7.86-7.75 (m, 2H), 7.74-7.57 (m, 2H), 7.47-7.21 (m, 4H), 4.51-4.15 (m, 4H), 3.93-3.69 (m, 2H), 3.44-3.31 (m, 2H), 1.13-0.90 (m, 1H), 0.58-0.39 (m, 2H), 0.31-0.08 (m, 2H).

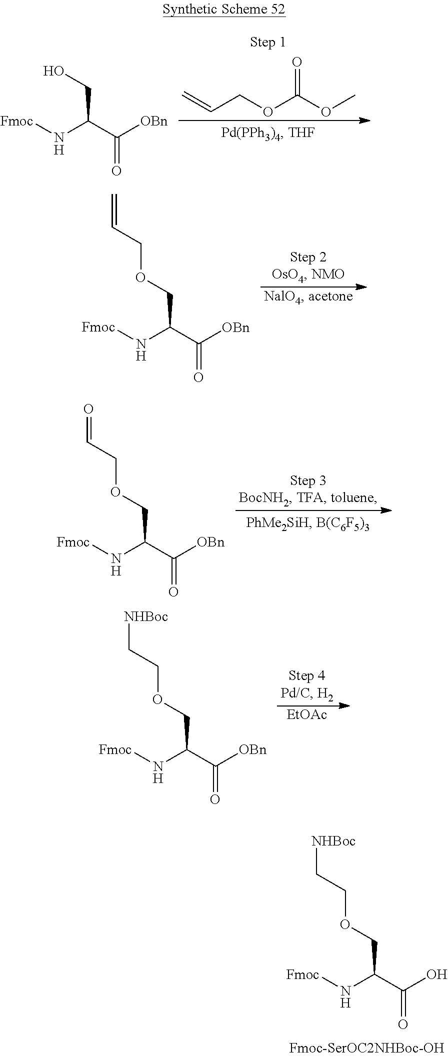

### N-(((9H-Fluoren-9-yl)methoxy)carbonyl)-O-(2-((tert-butoxycarbonyl)amino)ethyl)-L-serine (Fmoc-SerOC2NHBoc-OH)

Step 1: Allyl methyl carbonate (3.3 g, 28.7 mmol) and tetrakis(triphenylphosphine) palladium(0) (1.66 g, 1.44 mmol) were added to a stirred solution of benzyl (((9H-fluoren-9-yl) methoxy)carbonyl)-L-serinate (6.0 g, 14.4 mmol) in THF (120 mL) at RT under Ar. The resulting mixture was stirred o.n. at 65° C., then cooled to RT and concentrated under reduced pressure. The residue was purified by silica gel column chromatography, eluting with EtOAc in PE (1:2; v/v) to afford benzyl N-(((9H-fluoren-9-yl)methoxy)carbonyl)-O-allyl-L-serinate. MS ESI calculated for C₂₈H₂₈NO₅ [M+H]⁺ 458.19, found 458.05. ¹H NMR (400 MHz, chloroform-d): δ 7.80 (d, J=7.5 Hz, 2H), 7.66-7.64 (m, 2H), 7.45-7.33 (m, 9H), 5.87-5.75 (m, 2H), 5.28-5.16 (m, 4H), 4.65-4.56 (m, 1H), 4.49-4.36 (m, 2H), 4.29-4.25 (m, 1H), 4.04-3.89 (m, 3H), 3.75-3.72 (m, 1H).

Step 2: NMO (3.0 mL, 12.7 mmol, 50% in water) and OsO₄ (2.9 mL, 1.2 mmol, 10% in water) were added to a stirred solution of benzyl N-(((9H-fluoren-9-yl)methoxy)carbonyl)-O-allyl-L-serinate (5.3 g, 11.6 mmol) in acetone (53 mL) at 0° C. The resulting mixture was stirred at RT for 3 h. The reaction mixture was quenched with an aq. saturated NaHSO₃ solution (150 mL) and extracted with EtOAc (3×250 mL). The combined organic layers were washed with brine (250 mL) and dried over anh. Na₂SO₄. After filtration, the filtrate was concentrated under reduced pressure and the residue was dissolved in MeOH (53 mL). Sodium periodate (27.3 g, 12.7 mmol) was added to the solution and stirred o.n. at RT. The reaction mixture was quenched with water (250 mL) and extracted with Et₂O (3×250 mL). The combined organic layers were washed with brine (250 mL), dried over anh. Na₂SO₄. After filtration, the filtrate was concentrated under reduced pressure to give benzyl N-(((9H-fluoren-9-yl)methoxy)carbonyl)-O-(2-oxoethyl)-L-serinate. MS ESI calculated for C₂₇H₂₅NO₆Na [M+Na]⁺ 482.17, found 482.10.

Step 3: tert-Butyl carbamate (5.7 g, 48.3 mmol) and TFA (1.2 mL, 16.1 mmol) at RT were added to a mixture of benzyl N-(((9H-fluoren-9-yl)methoxy)carbonyl)-O-(2-oxoethyl)-L-serinate (3.7 g, 8.1 mmol) in toluene (40 mL). The reaction was stirred o.n. at RT. Dimethylphenylsilane (2.7 g, 20.1 mmol) and tris(pentafluorophenyl)borane (0.82 g, 1.6 mmol) were added to the reaction mixture. The solution was stirred o.n. at RT. The mixture was concentrated under reduced pressure and the residue was purified by silica gel column chromatography eluting with EtOAc in PE (1:2; v/v) to afford benzyl N-(((9H-fluoren-9-yl) methoxy)carbonyl)-O-(2-((tert-butoxycarbonyl)amino)ethyl)-L-serinate. MS ESI calculated for C₃₂H₃₆N₂O₇Na [M+Na]⁺ 583.25, found 583.15.

Step 4: Benzyl N-(((9H-fluoren-9-yl)methoxy)carbonyl)-O-(2-((tert-butoxycarbonyl) amino)ethyl)-L-serinate (2.2 g, 3.92 mmol) was dissolved in EtOAc (70 mL). The flask was evacuated and refilled with N₂ (5 times). Pd/C (300 mg, 0.28 mmol, dry, 10 wt. %) was added into the flask. The flask was evacuated and backfilled with H₂ (5 times). The resulting mixture was stirred for 4 h at RT under an atmosphere of dihydrogen. The flask was evacuated and refilled with N₂ (5 times). The resulting mixture was filtered through a Celite® pad. The filtered cake was washed with MeOH (3×100 mL). The filtrate was concentrated under reduced pressure. The residue was purified by silica gel column chromatography, eluting with MeOH in DCM (1:10; v/v) to afford N-(((9H-fluoren-9-yl)methoxy)carbonyl)-O-(2-((tert-butoxycarbonyl) amino)ethyl)-L-serine. MS ESI calculated for C₂₅H₃₁N₂O₇ [M+H]⁺ 471.21, found 471.10. ¹H NMR (300 MHz, DMSO-d₆): δ 12.75 (s, 1H), 7.90-7.88 (m, 2H), 7.74-7.62 (m, 3H), 7.42-7.39 (m, 2H), 7.39-7.33 (m, 2H), 6.83 (s, 1H), 4.34-4.15 (m, 4H), 3.72-3.68 (m, 2H), 3.45-3.40 (m, 2H), 3.15-3.00 (m, 2H), 1.37 (s, 9H).

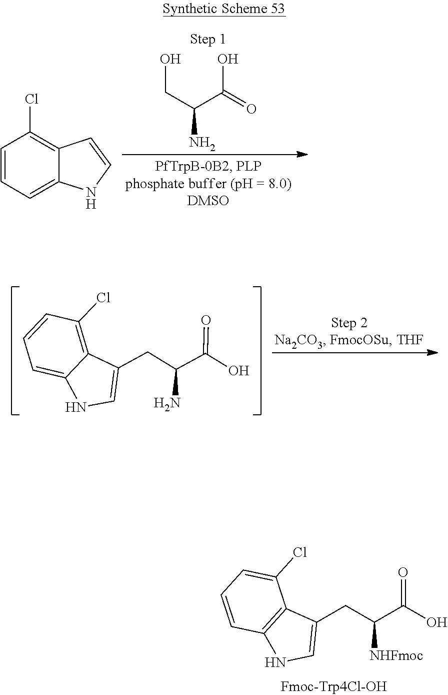

### (S)-2-((((9H-Fluoren-9-yl)methoxy)carbonyl)amino)-3-(4-chloro-1H-indol-3-yl)propanoic acid (Fmoc-Trp4Cl-OH)

Step 1: DMSO (120 mL), 4-chloro-1H-indole (17 g, 110 mmol), (S)-2-amino-3-hydroxypropanoic acid (12.37 g, 118 mmol), and 0.2 M potassium phosphate buffer (800 mL, 112 mmol, pH=8) were placed into a 2-L 4-necked round-bottom flask, purged and maintained with an inert atmosphere of N₂. The resulting solution was heated to 65° C. PLP (84.7 mg) in DI H₂O (2 mL) and PfTrpB-0B2 (1.19 g, 7 wt. %) in 0.2 M potassium phosphate buffer (2 mL) were added into the solution. The resulting solution was stirred o.n. at 65° C. Upon completion, the resulting solution was used directly in Step 2.

Step 2: Na₂CO₃ (23.68 g), Fmoc-OSu (40.88 g), and THF (240 mL) were added into the Step 1 reaction mixture at 0° C. The resulting solution was stirred for 2 h at RT. The solution was adjusted to pH=3 with an aq. 6 N HCl solution. Solids were removed by filtration. The mother liquor was extracted with EtOAc (3×500 mL). The combined organic layers were washed with DI water (2×1 L) and brine (1 L), dried over anh. Na₂SO₄, filtered, and concentrated under vacuum. The solids were purified by silica gel column chromatography, eluting with EtOAc 20% in DCM. The product fractions were collected and dried under reduced pressure. The product was then triturated with DCM (15V) and the solids were collected by filtration to give (S)-2-((((9H-fluoren-9-yl)methoxy)carbonyl)amino)-3-(4-chloro-1H-indol-3-yl)propanoic acid. MS ESI calculated for C₂₆H₂₁C₁N₂O₄ [M+H]⁺ 461, found 461. ¹H-NMR (400 MHz, DMSO-d₆): δ 12.63 (s, 1H), 11.23 (d, J=2.5 Hz, 1H), 7.92-7.81 (m, 2H), 7.74 (d, J=8.3 Hz, 1H), 7.65 (dd, J=17.6, 7.5 Hz, 2H), 7.50-7.22 (m, 6H), 7.09-6.97 (m, 2H), 4.34 (ddd, J=10.5, 8.2, 4.4 Hz, 1H), 4.26-4.13 (m, 3H), 3.56 (dd, J=14.6, 4.5 Hz, 1H), 3.14-2.92 (m, 1H).

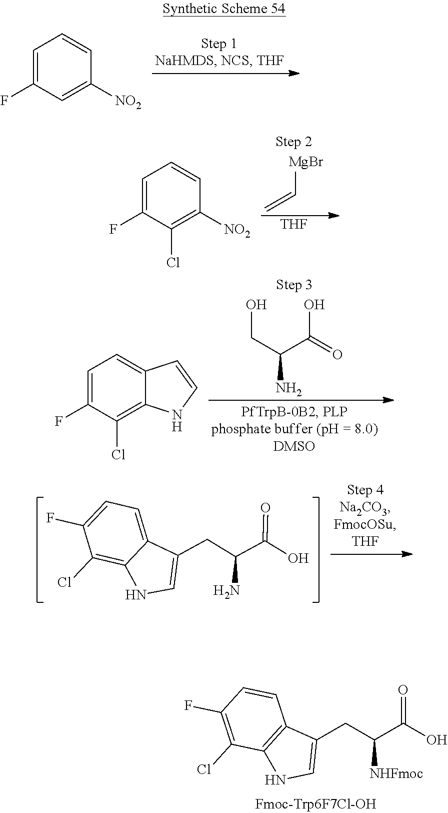

### (S)-2-((((9H-Fluoren-9-yl)methoxy)carbonyl)amino)-3-(7-chloro-6-fluoro-1H-indol-3-yl)propanoic acid (Fmoc-Trp6F7Cl-OH)

Step 1: THF (4 L, 10 V) and 3-fluoronitrobenzene (400 g, 2840 mmol) were added into a 4-necked round-bottom flask under N₂ and at RT. The flask was then cooled to −78° C. and 2.0 M NaHMDS in THF (2835 mL, 5700 mmol) was added dropwise. The resulting mixture was stirred for 1 h at −78° C. N-Chlorosuccinimide (1136 g, 8505 mmol) was then added at −78° C., and the resulting mixture was stirred at −78° C. for 3 h. The reaction was then quenched by the addition of aq. sat. NH₄C₁ solution (8 L) at −78° C. The resulting mixture was extracted with PE (3×3 L). The combined organic layers were then washed with aq. 1 N HCl (2×3 L), DI water (2×3 L), and brine (3 L), dried over anh. Na₂SO₄, filtered, and concentrated under vacuum to afford crude 2-chloro-1-fluoro-3-nitrobenzene.

Step 2: Anh. THF (5 L, 10V) under N₂ was added into a 20 L 4-necked round-bottom flask. 2-Chloro-1-fluoro-3-nitrobenzene (500 g, 2080 mmol) was added into the flask at RT. The reaction mixture was then cooled to −78° C. and 1.0 M vinylmagnesium bromide in THF (8.34 L, 8300 mmol) was added. The resulting mixture was stirred for 3 h at −78° C. The reaction was then quenched into aq. sat. NH₄Cl (5 L) at −78° C. The resulting mixture was extracted with EtOAc (3×5 L). The combined organic layers were washed with brine (5 L), dried over anh. Na₂SO₄, filtered, and concentrated under reduced pressure. The residue was purified by silica gel column chromatography, eluting with EtOAc 2% in PE to give 7-chloro-6-fluoro-1H-indole. ¹H NMR (400 MHz, DMSO-d₆): δ 11.63 (s, 1H), 7.53 (dd, J=8.6, 4.8 Hz, 1H), 7.43 (t, J=2.8 Hz, 1H), 7.05 (dd, J=10.1, 8.6 Hz, 1H), 6.55 (dd, J=3.1, 1.9 Hz, 1H).

Step 3: DMSO (1.6 L, 10V) under nitrogen atmosphere was placed into a 20 L 4-necked round-bottom flask. 7-Chloro-6-fluoro-1H-indole (157 g, 926 mmol) was added into the flask at RT, followed by L-serine (100.2 g, 953.6 mmol), and 0.2 M potassium phosphate buffer (pH=8, 6.4 L, 40V). Subsequently, PfTrpB-0B2(22 g, 14 wt. %) was added into the reactor at 65° C. PLP (1.37 g, 5.17 mmol) was then added into the reactor at 65° C., and the resulting mixture was stirred for 24 h at 65° C. The solution was used directly in the next step.

Step 4: THF (2.3 L, 17V) was added into the Step 3 solution at RT. The mixture was cooled to 0° C. and Na₂CO₃ (216.0 g, 2038 mmol) was added to the flask, followed by Fmoc-OSu (343.8 g, 1019 mmol). The ice bath was removed, and the mixture was stirred at RT for 3 h. The mixture was then cooled down to 5° C. and acidified to pH ˜4 with aq. 3 N HCl (3 L). The resulting mixture was diluted with EtOAc (10 L) and filtered through Celite® (500 g). The filtered cake was washed with EtOAc (2×10 L). The filtrate was then separated, and the aqueous phase was further extracted with EtOAc (10 L). The combined organic layers were washed with aq. 5% NaHCO₃ (3×10 L), aq. 1 N HCl (10 L), DI water (2×10 L), and brine (10 L). The organic phase was dried over anh. Na₂SO₄ and concentrated under reduced pressure. The crude was purified by trituration with DCM (5.6 L, 36V), and the cake was dried under vacuum at RT o.n. to yield (S)-2-((((9H-fluoren-9-yl)methoxy)carbonyl)amino)-3-(7-chloro-6-fluoro-1H-indol-3-yl)propanoic acid. MS ESI calculated for C₂₆H₂₀ClFN₂O₄[M+H]⁺ 479, found 479. ¹H NMR (400 MHz, DMSO-d₆): δ 12.50 (s, 1H), 11.23 (s, 1H), 7.82 (d, J=7.6 Hz, 2H), 7.59 (t, J=6.5 Hz, 2H), 7.50 (dt, J=13.2, 6.6 Hz, 2H), 7.36 (tt, J=7.6, 1.4 Hz, 2H), 7.29-7.18 (m, 3H), 6.97 (dd, J=10.1, 8.6 Hz, 1H), 4.24 (td, J=8.9, 4.8 Hz, 1H), 4.19 (d, J=3.1 Hz, 2H), 4.14 (d, J=7.0 Hz, 1H), 3.17 (dd, J=14.7, 4.9 Hz, 1H), 3.02 (dd, J=14.8, 9.4 Hz, 1H).

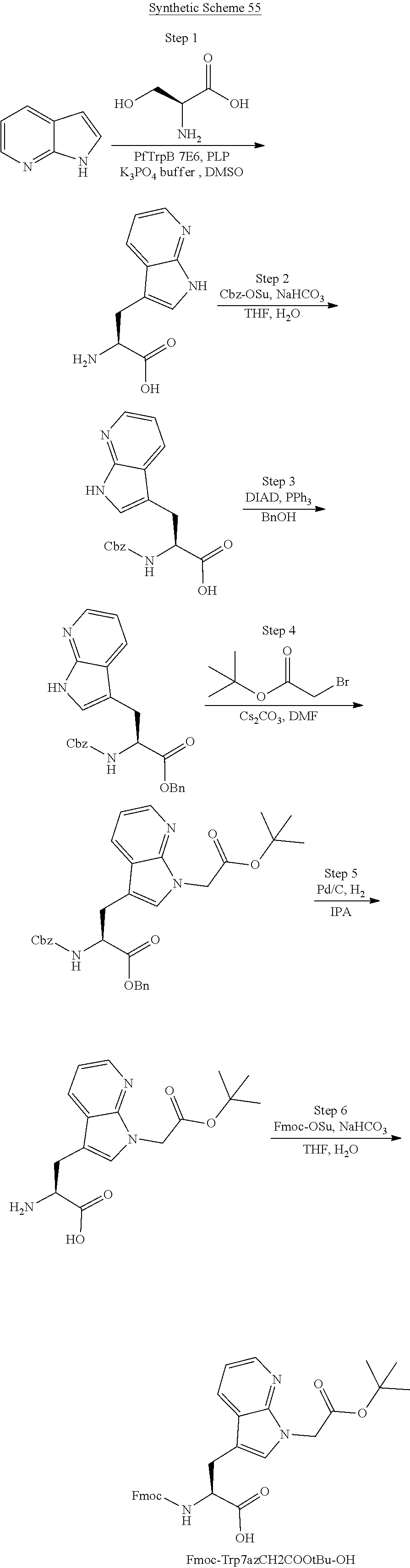

### (S)-2-((((9H-Fluoren-9-yl)methoxy)carbonyl)amino)-3-(1-(2-(tert-butoxy)-2-oxoethyl)-1H-pyrrolo[2,3-b]pyridin-3-yl)propanoic acid (Fmoc-Trp7azCH2COOtBu-OH)

Step 1: L-Serine (7.1 g, 67.7 mmol), (4-formyl-5-hydroxy-6-methylpyridin-3-yl)methyl dihydrogen phosphate (0.042 g, 0.17 mmol), and aldolase from Pseudomona Putida (0.80 g, 34 mmol) were added to a mixture of 1H-pyrrolo[2,3-b]pyridine (4.00 g, 33.9 mmol) in 1 M buffer (184 mL, pH=8.0, K₃PO₄) and DMSO (16 mL) at 65° C. The mixture was stirred o.n. at 65° C. The crude mixture of (S)-2-amino-3-(1H-pyrrolo[2,3-b]pyridin-3-yl)propanoic acid was used without workup or purification in Step 2. MS ESI calculated for C₁₀H₁₂N₃O₂ [M+H]⁺ 206.09, found 206.05.

Step 2: The crude mixture of (S)-2-amino-3-(1H-pyrrolo[2,3-b]pyridin-3-yl)propanoic acid (6.9 g, 34 mmol) from Step 1 was diluted with THF (200 mL) and water (200 mL). NaHCO₃(5.7 g, 67.2 mmol) and N-(benzyloxycarbonyloxy)succinimide (18.4 g, 74.0 mmol) were added to the mixture. The reaction mixture was stirred at RT for 4 h. The pH was adjusted to ˜3 with 1 N HCl. The mixture was extracted with EtOAc (2×100 mL). The combined organic layers were dried over anh. Na₂SO₄, filtered, and concentrated under reduced pressure. The residue was purified by silica gel chromatography, eluting with MeOH in DCM to afford (S)-2-(((benzyloxy) carbonyl)amino)-3-(1H-pyrrolo[2,3-b]pyridin-3-yl)propanoic acid. MS ESI calculated for C₁₈H₁₈N₃O₄ [M+H]⁺ 340.12, found 340.05. ¹H NMR (300 MHz, DMSO-d₆): δ 12.67 (s, 1H), 11.28 (s, 1H), 8.19-8.17 (m, 1H), 7.97-7.94 (m, 1H), 7.56 (d, J=8.2 Hz, 1H), 7.40-7.22 (m, 6H), 7.02-7.00 (m, 1H), 4.96 (s, 2H), 4.23-4.20 (m, 1H), 3.23-3.14 (m, 1H), 3.01-2.99 (m, 1H).

Step 3: Triphenylphosphine (3.7 g, 14.1 mmol), benzyl alcohol (1.9 g, 17.7 mmol), and DIAD (2.8 mL, 14.1 mmol) were added to a mixture of (S)-2-(((benzyloxy)carbonyl)amino)-3-(1H-pyrrolo[2,3-b]pyridin-3-yl)propanoic acid (4.0 g, 11.8 mmol) in anh. THF (80 mL) under an Ar atmosphere at RT. The mixture was stirred at RT for 3 h. The mixture was concentrated under reduced pressure and the residue was purified by silica gel chromatography, eluting with EtOAc in PE to afford benzyl (S)-2-(((benzyloxy)carbonyl)amino)-3-(1H-pyrrolo[2,3-b]pyridin-3-yl) propanoate. MS ESI calculated for C₂₅H₂₄N₃O₄ [M+H]⁺ 430.17, found 430.15.

Step 4: tert-Butyl 2-bromoacetate (2.1 g, 10.9 mmol) and Cs₂CO₃ (8.9 g, 27.2 mmol) were added to a solution of benzyl (S)-2-(((benzyloxy)carbonyl)amino)-3-(1H-pyrrolo[2,3-b]pyridin-3-yl)propanoate (3.9 g, 9.1 mmol) in anh. DMF (40 mL) under an Ar atmosphere at RT. The mixture was stirred at RT for 3 h. The mixture was diluted with EtOAc (550 mL). The organic layer was washed with brine (3×250 mL), dried over anh. Na₂SO₄, filtered, and concentrated under reduced pressure. The residue was purified by silica gel chromatography, eluting with EtOAc in PE to afford benzyl benzyl (S)-2-(((benzyloxy)carbonyl)amino)-3-(1-(2-(tert-butoxy)-2-oxoethyl)-1H-pyrrolo[2,3-b]pyridin-3-yl)propanoate. MS ESI calculated for C₃₁H₃₄N₃O₆[M+H]⁺ 544.24, found 544.20.

Step 5: Benzyl (S)-2-(((benzyloxy)carbonyl)amino)-3-(1-(2-(tert-butoxy)-2-oxoethyl)-1H-pyrrolo[2,3-b]pyridin-3-yl)propanoate (4.0 g, 7.4 mmol) was dissolved in isopropanol (80 mL). The flask was evacuated and refilled with N₂ (5 times). Pd/C (0.78 g, 7.4 mmol, dry, 10 wt. %) was added into the flask. The flask was evacuated and backfilled with H₂ (5 times). The resulting mixture was stirred for 4 h at RT under an atmosphere of dihydrogen. The flask was evacuated and refilled with N₂ (5 times). The resulting mixture was filtered through a Celite® pad. The filtered cake was washed with MeOH (3×50 mL). The filtrate was concentrated under reduced pressure to afford (S)-2-amino-3-(1-(2-(tert-butoxy)-2-oxoethyl)-1H-pyrrolo[2,3-b]pyridin-3-yl)propanoic acid. MS ESI calculated for C₁₆H₂₂N₃O₄ [M+H]⁺ 320.15, found 320.10.

Step 6: Fmoc-OSu (1.90 g, 5.64 mmol) and NaHCO₃ (2.63 g, 31.3 mmol) were added to a solution of (S)-2-amino-3-(1-(2-(tert-butoxy)-2-oxoethyl)-1H-pyrrolo[2,3-b]pyridin-3-yl) propanoic acid (2.0 g, 6.3 mmol) in THF (40 mL) and H₂O (40 mL) at RT. The mixture was stirred o.n. at RT. The pH of the reaction mixture was adjusted to ˜3 with 1 M HCl and then extracted with EtOAc (3×150 mL). The combined organic layers were washed with brine (3×50 mL), dried over anh. Na₂SO₄, filtered, and concentrated under reduced pressure. The residue was purified by Rp-flash column chromatography with the following conditions: Column: Flash C18; Mobile Phase A: water (0.1% TFA), Mobile Phase B: MeCN; Gradient Elution: 5-70%; Detector: UV 210 nm. The fractions containing the product were concentrated under reduced pressure to afford (S)-2-((((9H-fluoren-9-yl)methoxy)carbonyl)amino)-3-(1-(2-(tert-butoxy)-2-oxoethyl)-1H-pyrrolo[2,3-b]pyridin-3-yl)propanoic acid. MS ESI calculated for C₃₁H₃₂N₃O₆ [M+H]⁺ 542.22, found 542.15. ¹H NMR (400 MHz, DMSO-d₆): δ 12.85 (s, 1H), 8.23-8.21 (m, 1H), 8.07-8.05 (m, 1H), 7.94-7.84 (m, 3H), 7.71-7.64 (m, 2H), 7.51-7.04 (m, 6H), 4.94 (s, 2H), 4.34-4.11 (m, 4H), 3.23-3.18 (m, 1H), 3.08-3.02 (m, 1H), 1.38 (s, 9H).

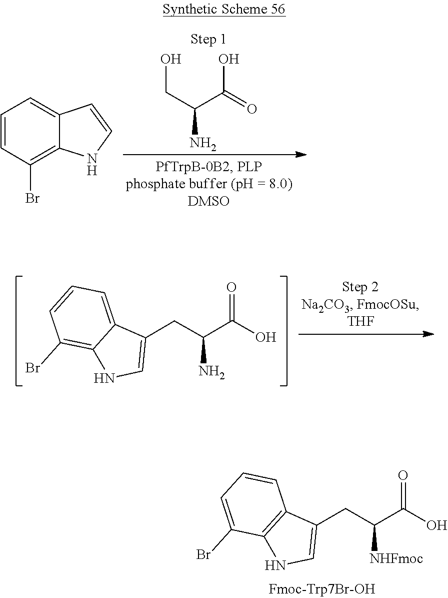

### (S)-2-((((9H-Fluoren-9-yl)methoxy)carbonyl)amino)-3-(7-bromo-1H-indol-3-yl)propanoic acid (Fmoc-Trp7Br-OH)

Step 1: DMSO (600 mL), 7-bromo-1H-indole (60 g, 300 mmol), (S)-2-amino-3-hydroxypropanoic acid (33.9 g, 322 mmol), 0.2 M potassium phosphate buffer (2400 mL, 40 V, pH=8.0) were placed into a 2-L 4-neck round-bottom flask, purged and maintained with an inert atmosphere of N₂. The resulting solution was stirred at 65° C. PLP (234 mg) in DI water (6 mL) and PfTrpB-0B2 (4.2 g, 7 wt. %) in 0.2 M potassium phosphate buffer (6 mL, pH=8) was added to the solution. The resulting solution was stirred o.n. at 65° C. The resulting solution was used directly in Step 2.

Step 2: Na₂CO₃ (63.9 g, 603 mmol), Fmoc-OSu (111.6 g, 331 mmol) and THF (360 mL) at 0° C. were added into the Step 1 solution. The resulting solution was stirred for 2 h at RT. The solution was adjusted to pH=3 with aq. 6 N HCl. Solids were removed by filtration. The mother liquor was extracted with EtOAc (3×500 mL). The combined organic layers were washed with DI water (3×6 L) and brine (3 L), dried over anh. Na₂SO₄, filtered, and concentrated under vacuum. The crude product was purified by silica gel column, eluting with EtOAc 50% in DCM. The product was then triturated with DCM (15V), and the solids were collected by filtration to afford (S)-2-((((9H-fluoren-9-yl)methoxy)carbonyl)amino)-3-(7-bromo-1H-indol-3-yl)propanoic acid. MS ESI calculated for C₂₆H₂₁BrN₂O₄ [M+H]⁺ 505, found 505. ¹H NMR (400 MHz, DMSO-d₆): δ 10.99 (s, 1H), 7.87 (d, J=7.6 Hz, 2H), 7.64-7.53 (m, 2H), 7.40 (t, J=7.5 Hz, 2H), 7.32-7.21 (m, 3H), 7.17 (d, J=2.4 Hz, 1H), 6.87 (t, J=7.7 Hz, 1H), 4.19-4.12 (m, 2H), 3.99 (d, J=5.8 Hz, 1H), 3.21 (dd, J=14.3, 4.6 Hz, 1H), 3.03 (dd, J=14.5, 7.4 Hz, 1H), 2.54 (s, 1H).

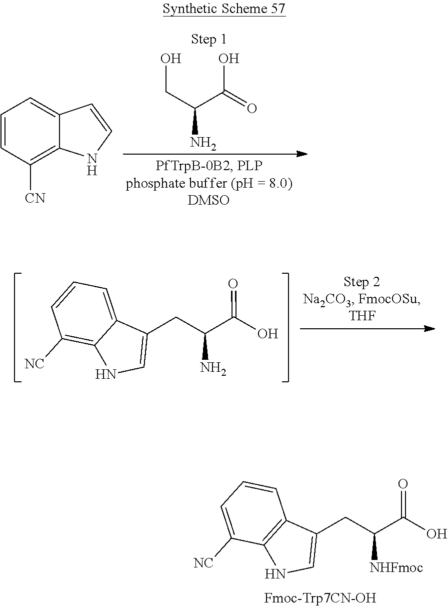

### (S)-2-((((9H-Fluoren-9-yl)methoxy)carbonyl)amino)-3-(7-cyano-1H-indol-3-yl)propanoic acid (Fmoc-Trp7CN-OH)

Step 1: Dimethyl sulfoxide (1.5 L, 10V) was added to a 20-L 4-neck round-bottom flask, purged and maintained with an inert atmosphere of N₂. Potassium phosphate buffer (0.2 M, pH=8, 6 L, 40V) was added to the flask at RT, followed by 1H-indole-7-carbonitrile (150 g, 1060 mmol) and L-serine (116.4 g, 1108 mmol), and the resulting mixture was heated to 65° C. under N₂. PfTrpB-0B2 (15 g, 10 wt. %) was then added into the flask, followed by PLP (1.56 g, 5.90 mmol), and the resulting mixture was stirred o.n. at 65° C. The resulting solution was used directly in Step 2.

Step 2: After 16 h, the Step 1 mixture was slowly cooled down to 0° C. THF (2.55 L, 17V) was added into the flask at 0° C., followed by Na₂CO₃ (248.4 g, 2321 mmol), and Fmoc-OSu (391.6 g, 1162 mmol). The resulting mixture was allowed to warm to RT and stirred for an additional 2 h. The mixture was then acidified to pH=4 with aq. 6 N HCl. The resulting mixture was extracted with EtOAc (3×6 L). The combined organic layers were washed with water (2×6 L) and brine (6 L), dried over anh. Na₂SO₄, filtered, and concentrated under reduced pressure. The crude was purified by trituration with a mixture of DCM (4.5 L, 3V) and hexane (4.5 L, 3V) at RT for 12 h. The precipitated solids were collected by filtration, washed with hexane (1 L), and dried at RT to give (S)-2-((((9H-fluoren-9-yl)methoxy)carbonyl)amino)-3-(7-cyano-1H-indol-3-yl)propanoic acid. MS ESI calculated for C₂₇H₂₁N₃O₄ [M+H]⁺ 452, found 452. ¹H NMR (400 MHz, DMSO-d₆): δ 12.81 (s, 1H), 11.84 (d, J=2.5 Hz, 1H), 7.97 (d, J=7.9 Hz, 1H), 7.88 (d, J=7.6 Hz, 2H), 7.79 (d, J=8.3 Hz, 1H), 7.69-7.56 (m, 3H), 7.49-7.35 (m, 3H), 7.29 (dtd, J=15.0, 7.5, 1.2 Hz, 2H), 7.17 (q, J=8.3, 7.7 Hz, 1H), 4.31-4.06 (m, 4H), 3.25 (dd, J=14.7, 4.6 Hz, 1H), 3.08 (dd, J=14.7, 9.9 Hz, 1H).

### Preparation of Final Compounds

### A. Generalized Procedure for Synthesizing Linear Peptide Precursors

Peptides in Table 1 and Table 2 were synthesized using standard solid-phase synthesis using Fmoc/tBu chemistry as exemplified in Chan, W. C.; White, P. D. “Fmoc Solid-Phase Synthesis: a Practical Approach”, Oxford University Press, Oxford, 2000; Steward, J.; Young, J. “Solid Phase Peptide Synthesis”, Pierce Chemical Company, Rockford, 1984; Benoiton, N. L. “Chemistry of Peptide Synthesis”, CRC Press, New York, 2006; and Lloyd-Williams, P.; Albericio, F.; Giralt, E. “Chemical Approaches to the Synthesis of Peptides and Proteins”, CRC Press, New York, 1997.

During peptide chain elongation, the α-amino group of each amino acid was protected with a 9H-fluoren-9-ylmethoxycarbonyl group (Fmoc). To avoid any side reactions during the chain elongation steps, any reactive amino acid side chains also carry acid-labile protecting groups, effectively masking the reactive groups until removal upon treatment with strong acid. After completion of each coupling step, the Fmoc group of the N-terminal amino acid was removed with piperidine or 4-methylpiperidine and the resin was thoroughly washed to prepare for the coupling of the subsequent Fmoc-protected amino acid derivative.

The side chain protecting groups used were:
tert-butyl (tBu) for L-Asp, L-Glu, α-Me-L-Ser, L-Ser, N-Me-L-Ser, hS, L-Thr, N-Me-L-Thr, L-Tyr, Phe4COOH, Phe3COOH, N-Me-L-Glu, hE, Apm, TyrOcm, SbMeY, RbMeY, TrpCH2COOH, NxG21, CeG, NhEG, F4MCO2H, BCP3CO2H, alT, Pipc5CO2H, AlaPentOH, RbOHPhe4COOH, dProt40AcOH, dProc40H, dProt40H, 3Pal4CO2H, AlaPyrim4COOH, Phe2F4COOH, Phe3F4COOH, Phe35F24COOH, Phe3Cl4COOH, RbOHPhe, SbMePhe4COOH, dProc4CH₂COOH;
trityl (Trt) for L-Asn, L-Cys, L-Gln, N-Me-L-Asn, NEtCONH2G, NEtCONH2A, dProt4Tet, SbOHAsn (with TBDMS-protected alcohol), RbOHAsn (with TBDMS-protected alcohol);
tert-butoxy-carbonyl (Boc) for L-Lys, L-Dab, L-Dap, L-Orn, hK, L-Trp, α-Me-L-Trp, L-His, Phe4CH₂NH₂, mAF, AlaPiperaz, AEF, Pip2c, NMeDab, DapMe, Prot4NH2, Pipc4NH2, dProc4NH2, dProt4NH2, dProt4CH2NH2, LysMe, Bay, SerOC2NH2, AlaPyrim4NH2 (bis-Boc);
and, 2,2,4,6,7-pentamethyldihydrobenzofuran-5-sulfonyl (Pbf) for L-Arg and hR.

Fmoc-protected amino acids were typically obtained from vendors such as Sigma-Aldrich® (Millipore Sigma, St. Louis, MO, USA), Novabiochem® (Millipore Sigma, St. Louis, MO, USA), Chem-Impex (Wood Dale, IL, USA), Combi-Blocks (San Diego, CA, USA), Ambeed (Arlington Hts, IL, USA), AstaTech, Inc. (Bristol, PA, USA), Iris Biotech (Marktredwitz, Germany), Acrotein BioChem, Inc. (Hoover, AL, USA), Amatek (Berwyn, PA, USA), ChemScene LLC (Monmouth Junction, NJ, USA), BLD Pharmatech Co., Limited (Cincinnati, OH, USA), AchemBlock (Hayward, CA, USA), AA Blocks LLC (San Diego, CA, USA), abcr GmbH (Karlsruhe, Germany), Enamine Ltd. (Kyiv, Ukraine), Chem Shuttle (Burlingame, CA, USA), or PharmaBlock, Inc. (Hatfield, PA, USA).

### B. Synthetic Procedures Used to Prepare Thioether Cyclic Peptides

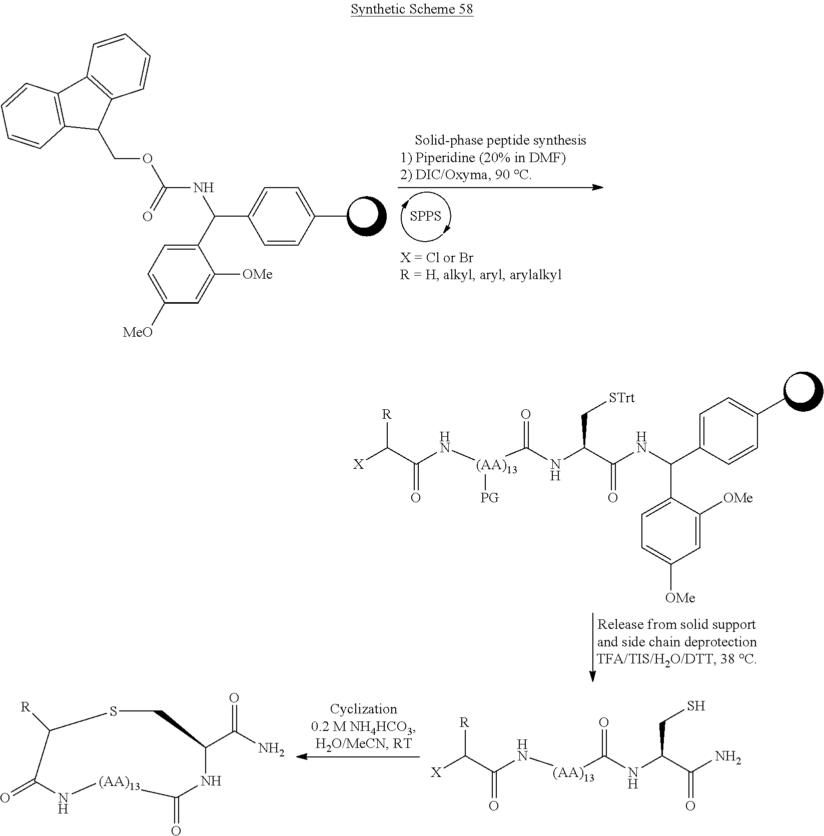

### 1. Procedure A

### a) Solid Phase Synthesis of Peptides (SPPS)

Peptides were synthesized on a Liberty Blue™ synthesizer from CEM Corporation (Matthews, NC), using standard solid-phase synthesis using Fmoc/tBu chemistry as summarized above in Synthetic Scheme 58.

Reactions were typically performed at a 50 μmol scale using Rink Amide MBHA LL resin (100-200 mesh, 0.4 mmol/g loading, 1% DVB cross-linked polystyrene, Novabiochem®). All the amino acids were dissolved at a 0.2 M concentration in anh. DMF or NMP. The amino acids were activated with equimolar amounts of Oxyma Pure solution (0.5 M in anh. DMF), and a 2-fold molar excess of DIC solution (0.5 M in anh. DMF).

Every synthesis cycle included: (1) Fmoc amino acid deprotection by 20% (v/v) piperidine or N-methylmorpholine (NMM) in anh. DMF (90° C. microwave assisted heating, 1 min); (2) Coupling (potentially repeated twice for difficult couplings) with Fmoc-protected amino acid/DIC/Oxyma (5, 5, and 10 equiv, respectively; 90° C. microwave assisted heating, 2 or 4 min). Cycles of Fmoc deprotection and Fmoc-protected amino acid coupling were repeated with the desired monomers until the full linear peptide was formed; (3) The peptide N-terminus was eventually capped with the required 2-chloroacetyl or 2-halo-2-substituted acetyl group by coupling with chloroacetic acid or the corresponding 2-bromo-2-substituted-acetic acid, respectively, and DIC (5 and 10 equiv, respectively; 90° C. microwave assisted heating, 2 min; repeated twice).

### b) Cleavage and Deprotection

After completion of the synthesis, the linear resin-bound peptide was transferred into a fritted plastic column. The resin was washed with DCM then dried under negative pressure on a Razor® peptide cleavage system from CEM Corporation (Matthews, NC). The peptide was deprotected and cleaved from the solid support by treatment with TFA/H₂O/TIS/DODT (92.5:2.5:2.5:2.5; v/v/v/v; 10 mL) at 38° C. for 30 min. The solution was collected into a 50 mL centrifuge tube. The resin washed with TFA (2×2 mL). The cleavage solution was partially concentrated to a volume of ˜5 mL under vacuum. The crude linear peptide was precipitated from the TFA cleavage solution using chilled tert-butyl methyl ether (MTBE; 40 mL) and collected by centrifugation (4000 rpm). The supernatant was removed. Additional cold MTBE (30 mL) was added to the peptide pellet and clarified by centrifugation. The resulting crude peptide pellet was blown dried by a stream of nitrogen gas.

### c) Peptide Cyclization

The crude solids were dissolved in 1:1 (v/v) mixture of DI water/MeCN (15 mL). The pH was adjusted to pH 8 by addition of an aq. 0.2 M ammonium bicarbonate solution (3 mL). The solution was shaken at RT for approximately 2 h and monitored by UPLC-MS. After the reaction was complete, the reaction solution was quenched by addition of TFA (200 μL), frozen and lyophilized.

### d) HPLC Purification

The crude residue was then dissolved in DMSO and purified by preparative reversed-phase high performance liquid chromatography on a Waters™ SunFire Prep C18 OBD column (100 Å, 5 μm, column size 19×150 mm, Milford, MA) using an Agilent MS-Directed Preparative HPLC-MS system. Mobile phase: (A) 0.1% TFA in HPLC-grade water and (B) 0.1% TFA in HPLC-grade acetonitrile; flow rate: 35 mL/min; UV wavelength λ=215 nm; gradient: 0.5% B/min for 40 min starting at 5% B less than the calculated % B required for compound elution. In some instances, better separation was obtained by 0.1% NH₄OH as modifier.

UV absorbing fractions containing the target m/z ions were collected and the fractions containing the desired product were combined, concentrated in vacuo, and freeze-dried to afford the cyclized peptide as a solid.

Confirmation of identity and purity assessment of final compounds were performed by UPLC-MS, which was measured by a Rp Waters™ ACQUITY UPLC-MS system. Column: Waters™ Cortecs C18+ Column (90 Å, 1.6 μm, column size 2.1×100 mm). Mobile phase: (A) 0.1% TFA in HPLC-grade water and (B) 0.1% TFA in HPLC-grade acetonitrile; injection volume: 0.5 μL; flow rate: 0.7 mL/min; Column Temperature: 60° C.; UV wavelength λ=215 nm; gradient: 2-35% in 9.1 min.

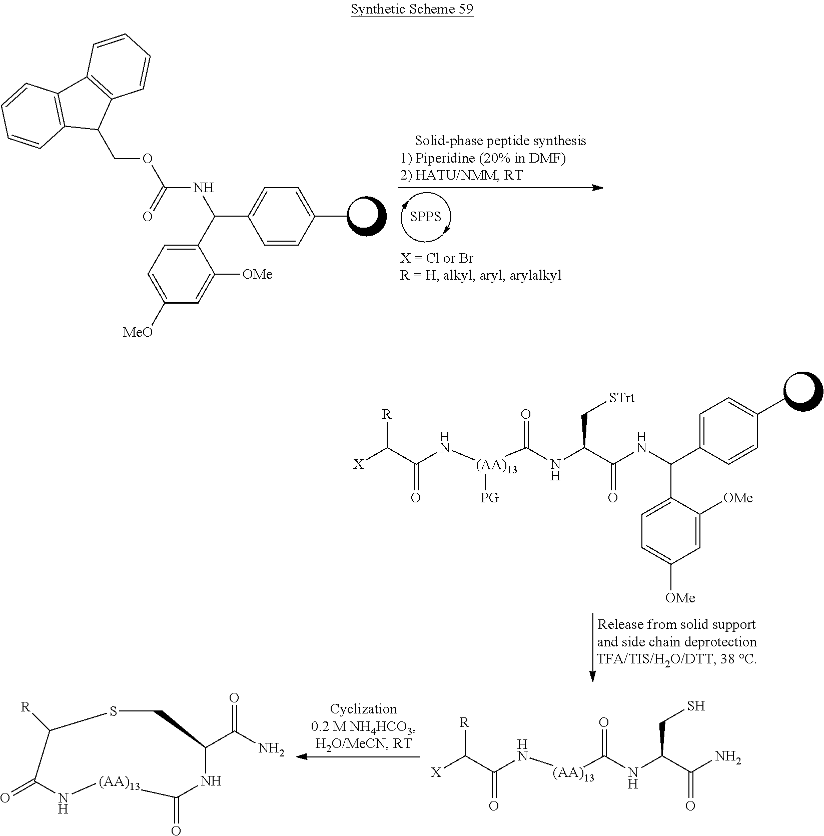

### 2. Procedure B

### a) Solid Phase Synthesis of Peptides (SPPS)

Peptides were synthesized on a Symphony® X synthesizer from Gyros Protein Technologies (Uppsala, Sweden), using standard solid-phase synthesis using Fmoc/tBu chemistry as summarized above in Synthetic Scheme 59.

Reactions were typically performed at a 100 μmol scale using Rink Amide MBHA LL resin (100-200 mesh, 0.4 mmol/g loading, 1% DVB cross-linked polystyrene, Novabiochem®). All the amino acids were dissolved at a 0.2 M concentration in anh. DMF or NMP. The amino acids were activated with equimolar amounts of HATU solution (0.5 M in anh. DMF) and a 2-fold molar excess of NMM solution (1.0 M in anh. DMF).

Every synthesis cycle included: (1) Fmoc amino acid deprotection by 20% (v/v) piperidine or 4-methylpiperidine in anh. DMF (RT; 3×3 min); (2) Coupling (potentially repeated twice for difficult couplings) with Fmoc-protected amino acid/HATU/NMM (5, 5, and 10 equiv, respectively; RT; 40 min). Cycles of Fmoc deprotection and Fmoc-protected amino acid coupling were repeated with the desired monomers until the full linear peptide was formed. Cycles of Fmoc deprotection and Fmoc-protected amino acid coupling were repeated with the desired monomers until the full linear peptide was formed; (3) The peptide N-terminus was eventually capped with the required 2-chloroacetyl by coupling with chloroacetic anhydride (1.0 M in anh. DMF) at RT for 10 min.

### b) Cleavage and Deprotection

After completion of the synthesis, the linear resin-bound peptide was transferred into a fritted plastic column. The resin was washed with DCM then dried under negative pressure on a Razor® peptide cleavage system from CEM Corporation (Matthews, NC). The peptide was deprotected and cleaved from the solid support by treatment with TFA/H₂OTIS/DTT (92.5:2.5:2.5:2.5; v/v/v/v; 10 mL) at 38° C. for 30 min. The solution was collected into a 50 mL centrifuge tube. The resin washed with TFA (2×2 mL). The cleavage solution was partially concentrated to a volume of −5 mL under vacuum. The crude linear peptide was precipitated from the TFA cleavage solution using chilled MTBE (40 mL) and collected by centrifugation (4000 rpm). The supernatant was removed. Additional cold MTBE (30 mL) was added to the peptide pellet and clarified by centrifugation. The resulting crude peptide pellet was blown dried by a stream of nitrogen gas.

### c) Peptide Cyclization

The crude solids were dissolved in 1:1 (v/v) mixture of DI water/MeCN (15 mL). The pH was adjusted to pH 8 by addition of an aq. 0.2 M ammonium bicarbonate solution (3 mL). The solution was shaken at RT for approximately 2 h and monitored by UPLC-MS. After the reaction was complete, the reaction solution was quenched by addition of TFA (200 μL), frozen and lyophilized.

### d) HPLC Purification

The crude residue was then dissolved in DMSO and purified by preparative reversed-phase high performance liquid chromatography on a Waters™ SunFire Prep C18 OBD column (100 Å, 5 μm, column size 19×150 mm) using an Agilent MS-Directed Preparative HPLC-MS system. Mobile phase: (A) 0.1% TFA in HPLC-grade water and (B) 0.1% TFA in HPLC-grade acetonitrile; flow rate: 35 mL/min; UV wavelength λ=215 nm; gradient: 0.5% B/min for 40 min starting at 5% B less than the calculated % B required for compound elution. In some instances, better separation was obtained by 0.1% NH₄OH as modifier.

UV absorbing fractions containing the target m/z ions were collected and the fractions containing the desired product were combined, concentrated in vacuo, and freeze-dried to afford the cyclized peptide as a solid.

Confirmation of identity and purity assessment of final compounds were performed by UPLC-MS, which was measured by a Rp Waters™ ACQUITY UPLC-MS system. Column: Waters™ Cortecs C18+ Column (90 Å, 1.6 μm, column size 2.1×100 mm). Mobile phase: (A) 0.1% TFA in HPLC-grade water and (B) 0.1% TFA in HPLC-grade acetonitrile; injection volume: 0.5 μL; flow rate: 0.7 mL/min; Column Temperature: 60° C.; UV wavelength λ=215 nm; gradient: 2-35% in 9.1 min.

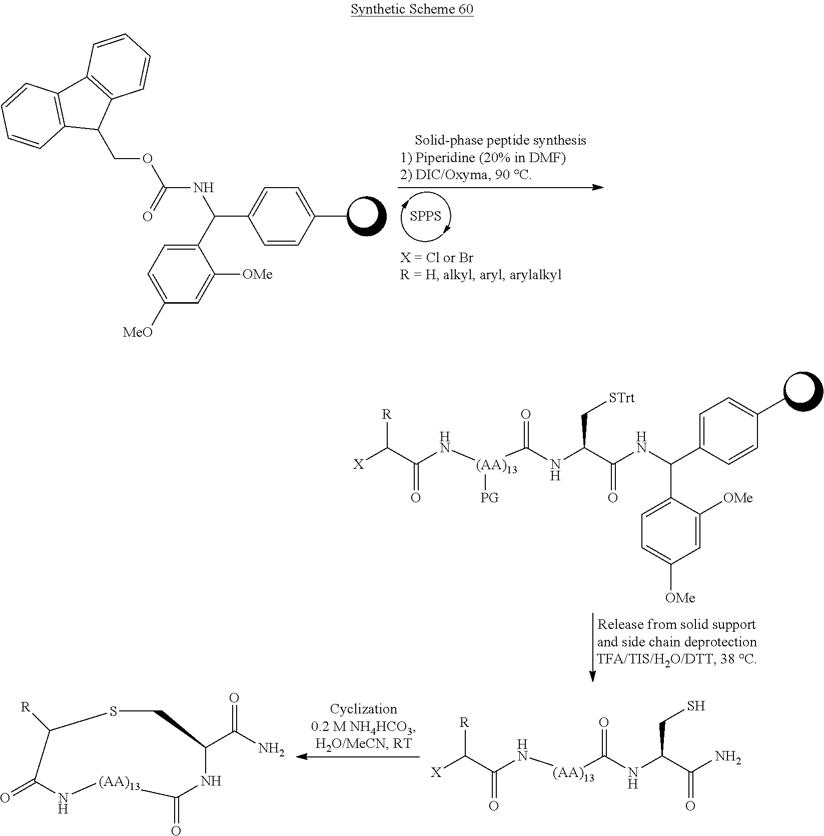

### 3. Procedure C

### a) Solid Phase Synthesis of Peptides (SPPS)

Peptides were synthesized on a Liberty Blue™ synthesizer from CEM Corporation (Matthews, NC), using standard solid-phase synthesis using Fmoc/tBu chemistry as summarized above in Scheme 60.

Reactions were typically performed at the 25 μmol or 50 μmol scale using Rink Amide MBHA LL resin (100-200 mesh, 0.4 mmol/g loading, Novabiochem®, 1% DVB cross-linked polystyrene). Solutions of the reagents and amino acid monomers were prepared as follows:
25 μmol scale: Fmoc-protected amino acids (0.2 M in anh. DMF, NMP, or DMSO), Oxyma Pure (0.25 M in anh. DMF), DIC (0.5 M in anh. NMP), (ClAc)₂O (10% w/v in anh. DMF), 4-methylpiperidine (20% (v/v) in anh. DMF+0.1 M Oxyma Pure),
50 μmol scale: -Fmoc-protected amino acids (0.2 M in anh. DMF, NMP, or DMSO), Oxyma Pure (0.5 M in anh. DMF), DIC (1.0 M in anh. NMP), (ClAc)₂O (10% w/v in anh. DMF), 4-methylpiperidine (20% (v/v) in anh. DMF+0.1 M Oxyma Pure).

Every synthesis cycle included: (1) Single or double coupling with Fmoc-protected amino acid/DIC/Oxyma (5, 10 and 5 equiv, respectively) at 90° C. using microwave-assisted heating, for 2 or 4 min, or 50° C. using microwave-assisted heating for 10 min. After the coupling reaction was complete, the mixture was filtered, and the peptidyl resin was washed with DMF; (2) Fmoc deprotection (repeated three times) using 20% (v/v) 4-methylpiperidine in anh. DMF (90° C. microwave assisted heating; 3 min). The mixture was filtered, and the peptidyl resin was washed with DMF. Cycles of Fmoc deprotection and Fmoc-protected amino acid coupling were repeated with the desired monomers until the full linear peptide was formed; (3) The peptide N-terminus was capped by the addition of (ClAc)₂O (10 equiv, repeated twice), then the peptidyl resin was washed with DMF.

### b) Cleavage and Deprotection

After completion of the synthesis, the linear resin-bound peptide was transferred into a fritted plastic column. The resin was washed with DCM then dried under negative pressure on a Razor® peptide cleavage system from CEM Corporation (Matthews, NC). The peptide was deprotected and cleaved from the solid support by treatment with TFA/H₂O/TIS/DODT (92.5:2.5:2.5:2.5; v/v/v/v; 10 mL) at 40° C. for 30 min. The solution was collected into a 50 mL centrifuge tube. The resin washed with TFA (2×2 mL). The cleavage solution was partially concentrated to a volume of ˜5 mL under vacuum. The crude linear peptide was precipitated from the TFA cleavage solution using Et₂O (40 mL) and collected by centrifugation (4000 rpm). The supernatant was removed. Additional cold Et₂O (30 mL) was added to the peptide pellet and clarified by centrifugation. The resulting crude peptide pellet was blown dried by a stream of nitrogen gas.

### c) Peptide Cyclization

The crude solids were dissolved in 1:1 (v/v) mixture of DI water/MeCN (15 mL). The pH was adjusted to pH 8 by addition of an aq. 0.2 M ammonium bicarbonate solution (3 mL). The solution was shaken at RT for approximately 12 h and monitored by UPLC-MS. After the reaction was complete, the reaction solution was quenched by addition of AcOH (200 μL), frozen and lyophilized. The resulting crude peptide pellet was re-dissolved into DMSO and submitted for RP-purification.

### d) HPLC Purification

Purification was performed by preparative reversed-phase high performance liquid chromatography (Rp-HPLC) on Waters™ X-Bridge Prep C18 OBD Prep column (130 Å, 5 μm, column size 19×100 mm) using a Waters™ MS-Directed AutoPurification HPLC-MS system. Mobile phase: (A) 0.16% TFA in HPLC-grade water and (B) 0.16% TFA in HPLC-grade acetonitrile; flow rate: 25 mL/min; UV wavelength λ=215 nm; gradient: 25-50% B over 5 min. Alternatively purification was performed on Waters™ CSH-C18 Column (19×250 mm, 5 μm) using an Agilent, with 1290 infinity II preparative LC system and LC-MSD XT mass spectrometer. Mobile phase: (A) 0.1% formic acid in HPLC-grade water and (B) 0.1% formic acid in HPLC-grade acetonitrile; flow rate: 25 mL/min; UV wavelength λ=215 nm; gradient: 20% B over 2.5 min, then increasing to 55% B over the next 17.5 min. UV absorbing fractions containing the target m/z ions were collected and the fractions containing product were confirmed by LC-MS.

Confirmation of identity and purity assessment of final compounds were performed by UPLC-MS, which was measured by a Rp Waters™ ACQUITY UPLC-MS system. Column: Waters™ XSelect CSH C18 Column (130 Å, 2.5 μm, column size 2.1×50 mm). Mobile phase: (A) 0.05% TFA in HPLC-grade water and (B) 0.05% TFA in HPLC-grade acetonitrile; injection volume: 1 μL; flow rate: 1 mL/min; UV wavelength λ=215 nm; gradient: 5-100% B in 5 min. Lyophilization of combined fractions containing pure peptide resulted in the final cyclized product as a powder.

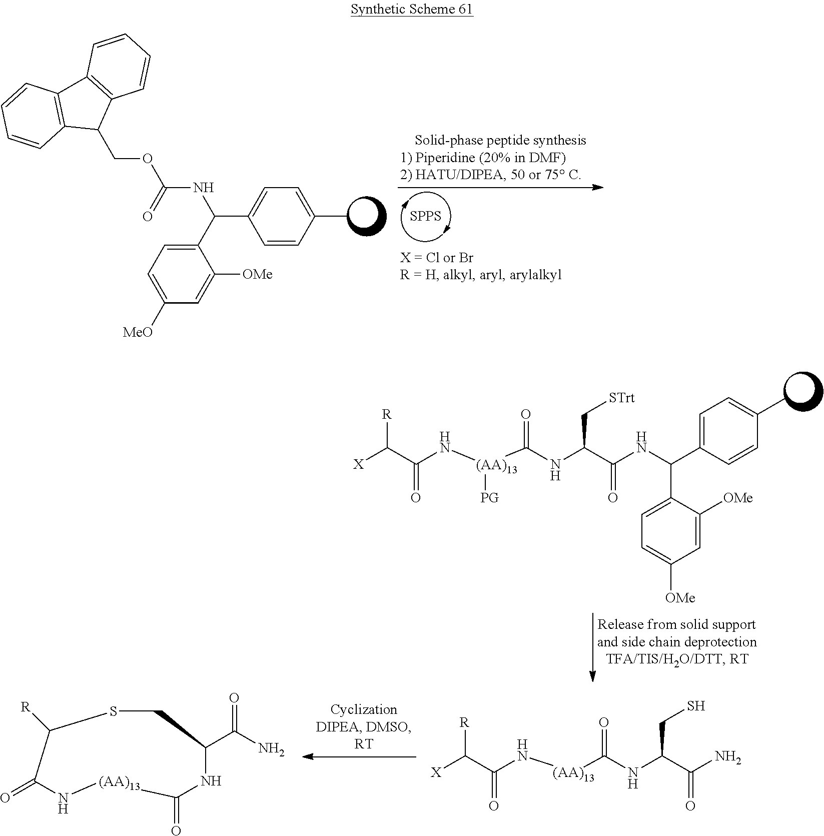

### 4. Procedure D

### a) Solid Phase Synthesis of Peptides (SPPS)

Peptides were synthesized on a Biotage® Syro II peptide synthesizer (Biotage Corporation, Boston, MA) using standard solid-phase synthesis using Fmoc/tBu chemistry as summarized above in Synthetic Scheme 61.

Reactions were typically performed at the 10 μmol or 25 μmol scale using Rink Amide MBHA LL resin (100-200 mesh, 0.4 mmol/g loading, Novabiochem®). Solutions of the reagents and amino acid monomers were prepared as follows: Fmoc-protected amino acids (0.2 M or 0.4 M in anh. DMF, NMP, or DMSO), HATU (0.5 M in anh. DMF), DIPEA (2.0 M in anh. NMP), (ClAc)₂O (0.4 M in anh. DMF), 4-methylpiperidine (20% (v/v) in anh. DMF).

Every synthesis cycle included: (1) Single or double coupling with Fmoc-protected amino acid/HATU/DIPEA (4, 4 and 8 equiv, respectively; at 50° C. or 75° C.; 15 min for single coupling or 15 min+30 min for double coupling). After the coupling reaction was complete the mixture was filtered, and the peptidyl resin was washed with DMF; (2) Fmoc deprotection (repeated three times) using 20% (v/v) 4-methylpiperidine in DMF (RT; 3 min). The mixture was filtered, and the peptidyl resin was washed with DMF. Cycles of Fmoc deprotection and Fmoc-protected amino acid coupling were repeated with the desired monomers until the full linear peptide was formed; (3) The peptide N-terminus was capped by the addition of (ClAc)₂O and DIPEA (10 and 20 equiv, respectively; repeated twice) in anh. DMF, then the peptidyl resin was washed with DCM.

### b) Cleavage and Deprotection

After completion of the synthesis, the linear resin-bound peptide was washed with DCM then dried under positive nitrogen pressure on a peptide cleavage system from Biotage Corporation, Boston, MA.

The peptide was deprotected and cleaved from the solid support by treatment with TFA/H₂O/TIS/DODT (92.5:2.5:2.5:2.5; v/v/v/v; 2 mL) at RT for 1 h. The solution was collected into a 50 mL centrifuge tube. The resin washed with TFA (1 mL). The crude linear peptide was precipitated from the TFA cleavage solution using chilled Et₂O (30 mL) and collected by centrifugation (4000 rpm). The supernatant was removed. Additional cold Et₂O (30 mL) was added to the peptide pellet and clarified by centrifugation. The resulting crude peptide pellet was blown dried by a stream of nitrogen gas.

### c) Peptide Cyclization

The crude solids were re-dissolved in DMSO (1.5 mL). The pH was adjusted by addition of 2.0 M DIPEA in NMP solution (0.5 mL). The solution was shaken at RT for approximately 12 h and monitored by UPLC-MS. After the reaction was complete, the cyclization mixture was acidified by the addition of 40 μL glacial acetic acid and the resulting solutions were filtered and submitted for Rp purification.

### d) HPLC Purification

Purification was performed by preparative reversed-phase high performance liquid chromatography (Rp-HPLC) on Waters™ X-Bridge Prep C18 OBD Prep column (130 Å, 5 μm, column size 19×100 mm) using a Waters™ MS-Directed AutoPurification HPLC-MS system. Mobile phase: (A) 0.16% TFA in HPLC-grade water and (B) 0.16% TFA in HPLC-grade acetonitrile; flow rate: 25 mL/min; UV wavelength λ=215 nm; gradient: 25-50% B over 5 min. Alternatively purification was performed on Waters™ CSH-C18 Column (19×250 mm, 5 μm) using an Agilent, with 1290 infinity II preparative LC system and LC-MSD XT mass spectrometer. Mobile phase: (A) 0.1% formic acid in HPLC-grade water and (B) 0.1% formic acid in HPLC-grade acetonitrile; flow rate: 25 mL/min; UV wavelength λ=215 nm; gradient: 20% B over 2.5 min, then increasing to 55% B over the next 17.5 min. UV absorbing fractions containing the target m/z ions were collected and the fractions containing product were confirmed by LC-MS.

Confirmation of identity and purity assessment of final compounds were performed by UPLC-MS, which was measured by a Rp Waters™ ACQUITY UPLC-MS system. Column: Waters™ XSelect CSH C18 Column (130 Å, 2.5 μm, column size 2.1×50 mm). Mobile phase: (A) 0.05% TFA in HPLC-grade water and (B) 0.05% TFA in HPLC-grade acetonitrile; injection volume: 1 μL; flow rate: 1 mL/min; UV wavelength λ=215 nm; gradient: 5-100% B in 5 min. Lyophilization of combined fractions containing pure peptide resulted in the final cyclized product as a powder.

C. Synthetic Procedures used to Prepare Macrolactam Cyclic Peptides

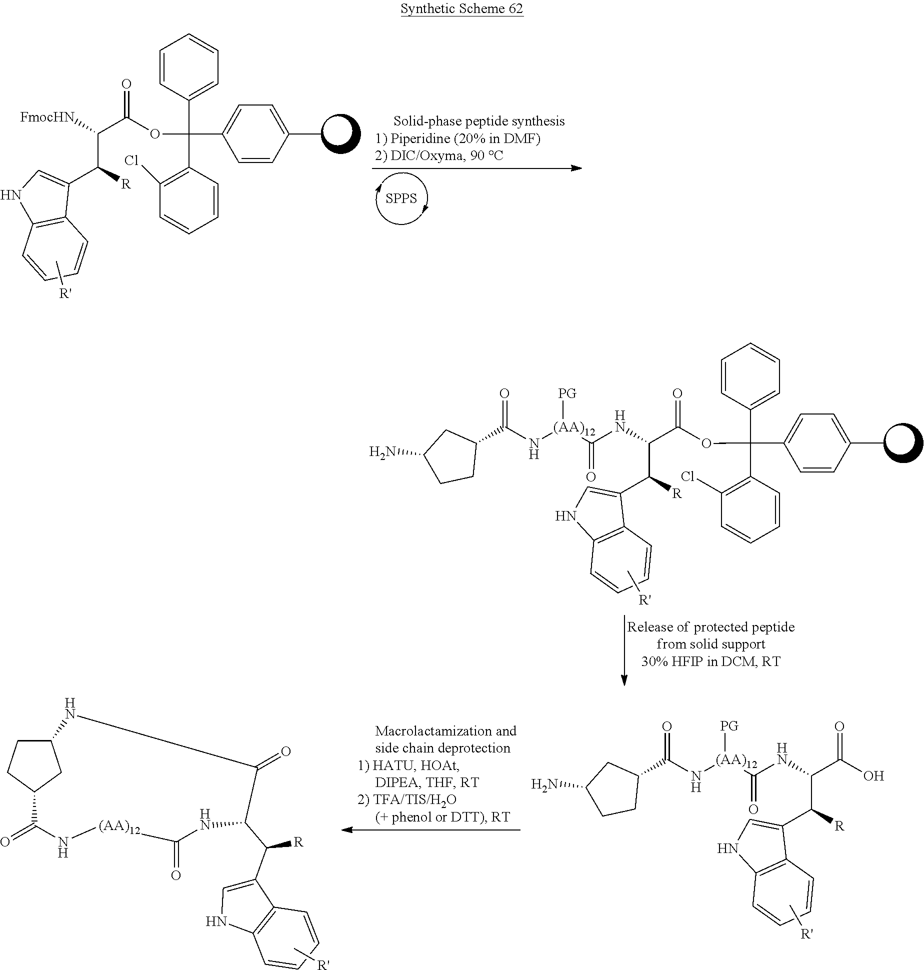

### 1. Procedure E

### a) Solid Phase Synthesis of Peptides (SPPS)

Peptides were synthesized on a Liberty Blue™ synthesizer from CEM Corporation (Matthews, NC), using standard solid-phase synthesis using Fmoc/tBu chemistry as summarized above in Synthetic Scheme 62.

Reactions were typically performed at a 50 μmol scale using Cl-TCP(Cl) ProTide® resin (100-200 mesh, 0.37 mmol/g loading, CEM Corporation, Matthews, GA). All the amino acids were dissolved at a 0.2 M concentration in anh. DMF or NMP. The amino acids were activated with equimolar amounts of Oxyma Pure solution (0.5 M in anh. DMF containing 0.1 M of DIPEA), and a 2-fold molar excess of DIC solution (0.5 M in anh. DMF).

The loading of the initial C-terminal amino acid to the Cl-TCP(Cl) ProTide® resin was performed directly on the Liberty Blue™ synthesizer by treating with 10-fold molar excess of Fmoc-protected amino acid solution (0.2 M in anh. DMF) and 20-fold molar excess of base solution (1.0 M DIPEA+0.125 M KI in anh. DMF) and 90° C. microwave assisted heating for 10 min (repeated twice).

Every synthesis cycle included: (1) Fmoc amino acid deprotection by 20% (v/v) piperidine in anh. DMF (90° C. microwave assisted heating, 1 min); (2) Coupling (potentially repeated twice for difficult couplings) with Fmoc-protected amino acid/DIC/Oxyma (5, 5, and 10 equiv, respectively; 90° C. microwave assisted heating, 2 min). Cycles of Fmoc deprotection and Fmoc-protected amino acid coupling were repeated with the desired monomers until the full linear protected peptide was formed; (3) The peptide N-terminus was deprotected using 20% (v/v) piperidine in DMF (50° C. microwave assisted heating, 10 min; repeated twice).

### b) Cleavage of Protected Linear Peptide

After completion of the synthesis, the linear resin-bound peptide was transferred into a fritted plastic column. The resin was washed with DCM then dried under negative pressure on a Razor® peptide cleavage system from CEM Corporation (Matthews, NC). The protected peptide was cleaved off the solid support by treatment with HFIP/DCM (3:7; v/v; 5 mL) at RT for 5 min. The solution was collected into a 50 mL centrifuge tube. This process was repeated twice more (three times total), and the cleavage solution was combined into the same tube. The solvents were removed under reduced pressure.

### c) Solution Phase Macrolactamization

The crude protected linear peptide was resuspended in anh. THF (10 mL). To that solution was added DIPEA (45 μL, 5 equiv) followed by the dropwise addition of the solution of HOAt (6 mg, 0.9 equiv) and HATU (8.4 mg, 1.3 equiv) dissolved in anh. DMF (150 μL). The solution was shaken at RT for approximately 30 min and monitored by UPLC-MS. After the reaction was complete, the reaction solution was concentrated under reduced to dryness.

### d) Deprotection of the Protected Cyclic Peptide

The cyclic peptide was deprotected by treatment with TFA/H₂O/TIS/Phenol (93:2:3:2; v/v/v/w; 7 mL) at RT for 1 h. The crude cyclic peptide was precipitated from the TFA cleavage solution using chilled MTBE (40 mL) and collected by centrifugation (4000 rpm). The supernatant was removed. Additional cold MTBE (35 mL) was added to the peptide pellet and clarified by centrifugation. The resulting crude peptide pellet was dissolved in 1:1 (v/v) mixture of DI water/MeCN (10 mL). The solution was frozen and lyophilized.

### e) HPLC Purification

The crude residue was then dissolved in DMSO and purified by preparative reversed-phase high performance liquid chromatography on a Waters™ SunFire Prep C18 OBD column (100 Å, 5 μm, column size 19×150 mm, Milford, MA) using an Agilent MS-Directed Preparative HPLC-MS system. Mobile phase: (A) 0.1% TFA in HPLC-grade water and (B) 0.1% TFA in HPLC-grade acetonitrile; flow rate: 35 mL/min; UV wavelength λ=215 nm; gradient: 0.5% B/min for 40 min starting at 5% B less than the calculated % B required for compound elution. In some instances, better separation was obtained by 0.1% NH₄OH as modifier.

UV absorbing fractions containing the target m/z ions were collected and the fractions containing the desired product were combined, concentrated in vacuo, and freeze-dried to afford the cyclized peptide as a solid.

Confirmation of identity and purity assessment of final compounds were performed by UPLC-MS, which was measured by a Rp Waters™ ACQUITY UPLC-MS system. Column: Waters™ Cortecs C18+ Column (90 Å, 1.6 μm, column size 2.1×100 mm, Milford, MA). Mobile phase: (A) 0.1% TFA in HPLC-grade water and (B) 0.1% TFA in HPLC-grade acetonitrile; injection volume: 0.5 μL; flow rate: 0.7 mL/min; Column Temperature: 60° C.; UV wavelength λ=215 nm; gradient: 2-35% in 9.1 min.

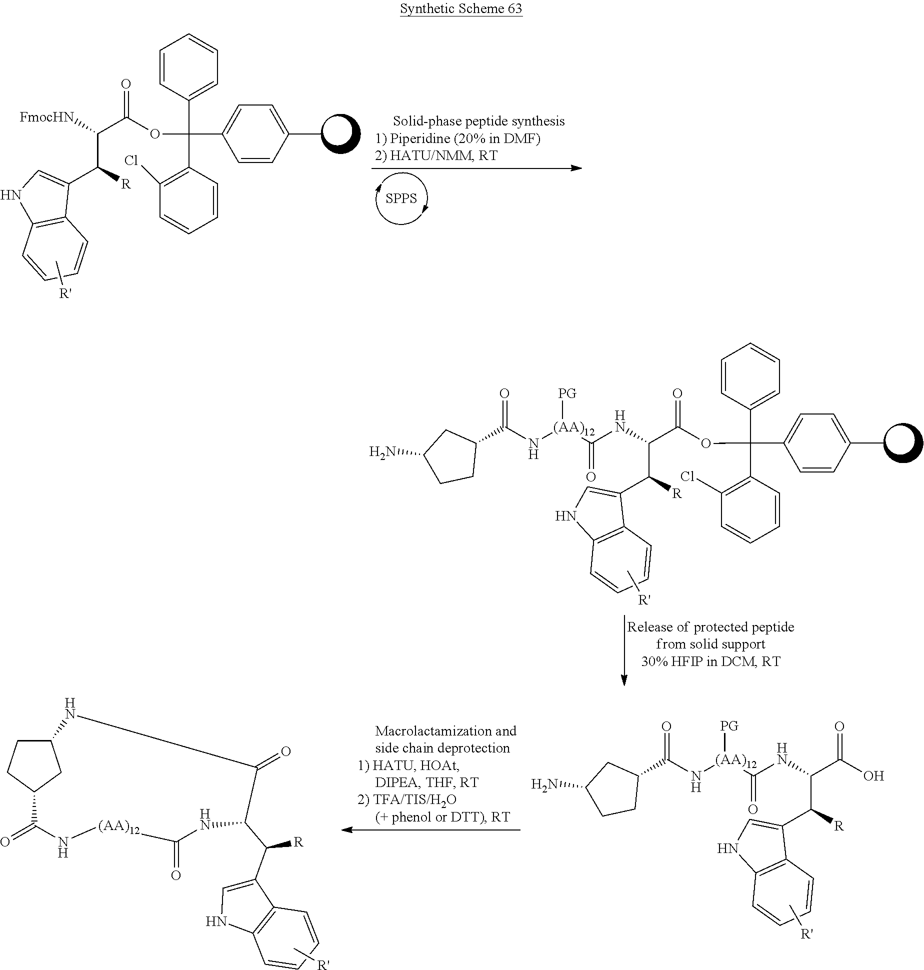

### 2. Procedure F

### a) Solid Phase Synthesis of Peptides (SPPS)

Peptides were synthesized on a Symphony® X synthesizer from Gyros Protein Technologies (Uppsala, Sweden), using standard solid-phase synthesis using Fmoc/tBu chemistry as summarized above in Synthetic Scheme 63.

Reactions were typically performed at a 50 μmol scale using Cl-TCP(Cl) ProTide® resin (100-200 mesh, 0.37 mmol/g loading, CEM). All the amino acids were dissolved at a 0.2 M concentration in anh. DMF or NMP. The amino acids were activated with equimolar amounts of HATU solution (0.5 M in anh. DMF) and a 2-fold molar excess of NMM solution (1.0 M in anh. DMF).

The loading of the initial C-terminal amino acid to the Cl-TCP(Cl) ProTide® resin was performed directly on the Symphony® X synthesizer by treating with 2-fold molar excess of Fmoc-protected amino acid solution (0.2 M in anh. DMF) and 4-fold molar excess of DIPEA solution (1.0 M in anh. DMF) at RT for 2 h (repeated twice). The unreacted resin was eventually capped by treatment with MeOH.

Every synthesis cycle included: (1) Fmoc amino acid deprotection by 20% (v/v) piperidine in anh. DMF (RT; 3×5 min) and coupling (potentially repeated twice for difficult couplings) with Fmoc-protected amino acid/HATU/NMM (4, 4, and 8 equiv respectively; RT; 30 min); (2) Cycles of Fmoc deprotection and Fmoc-protected amino acid coupling were repeated with the desired monomers until the full linear peptide was formed. Cycles of Fmoc deprotection and Fmoc-protected amino acid coupling were repeated with the desired monomers until the full linear peptide was formed; (3) The peptide N-terminus was deprotected using 20% (v/v) piperidine in DMF (RT; 3×5 min).

### b) Cleavage of Protected Linear Peptide

After completion of the synthesis, the linear resin-bound peptide was transferred into a fritted plastic column. The resin was washed with DCM then dried under negative pressure on a Razor® peptide cleavage system from CEM Corporation. The protected peptide was cleaved off the solid support by treatment with HFIP/DCM (3:7; v/v; 4 mL) at RT for 5 min. The solution was collected into a 50 mL centrifuge tube. This process was repeated twice more (three times total), and the cleavage solution was combined into the same tube. The solvents were removed under reduced pressure.

### c) Solution Phase Macrolactamization

The crude protected linear peptide was resuspended in anh. THF (8 mL). To that solution was added DIPEA (22 μL, 2.5 equiv) followed by the dropwise addition of the solution of HOAt (6 mg, 0.9 equiv) and HATU (8.4 mg, 1.3 equiv) dissolved in anh. DMF (300 μL). The solution was shaken at RT for approximately 15 min and monitored by UPLC-MS. After the reaction was complete, the reaction solution was concentrated under reduced to dryness.

### d) Deprotection of the Protected Cyclic Peptide

The cyclic peptide was deprotected by treatment with TFA/H₂OTIS/DTT (94:2.5:2.5:1; v/v/v/v; 5 mL) at RT for 1 h. The crude cyclic peptide was precipitated from the TFA cleavage solution using chilled MTBE (40 mL) and collected by centrifugation (4000 rpm). The supernatant was removed. Additional cold MTBE (35 mL) was added to the peptide pellet and clarified by centrifugation. The resulting crude peptide pellet was dissolved in 1:1 (v/v) mixture of DI water/MeCN (10 mL). The solution was frozen and lyophilized.

### e) HPLC Purification

The crude residue was then dissolved in DMSO and purified by preparative reversed-phase high performance liquid chromatography on a Waters™ SunFire Prep C18 OBD column (100 Å, 5 μm, column size 19×150 mm, Milford, MA) using an Agilent MS-Directed Preparative HPLC-MS system. Mobile phase: (A) 0.1% TFA in HPLC-grade water and (B) 0.1% TFA in HPLC-grade acetonitrile; flow rate: 35 mL/min; UV wavelength λ=215 nm; gradient: 0.5% B/min for 40 min starting at 5% B less than the calculated % B required for compound elution. In some instances, better separation was obtained by 0.1% NH₄OH as modifier.

UV absorbing fractions containing the target m/z ions were collected and the fractions containing the desired product were combined, concentrated in vacuo, and freeze-dried to afford the cyclized peptide as a solid.

Confirmation of identity and purity assessment of final compounds were performed by UPLC-MS, which was measured by a Rp Waters™ ACQUITY UPLC-MS system. Column: Waters™ Cortecs C18+ Column (90 Å, 1.6 μm, column size 2.1×100 mm, Milford, MA). Mobile phase: (A) 0.1% TFA in HPLC-grade water and (B) 0.1% TFA in HPLC-grade acetonitrile; injection volume: 0.5 μL; flow rate: 0.7 mL/min; Column Temperature: 60° C.; UV wavelength λ=215 nm; gradient: 2-35% in 9.1 min.

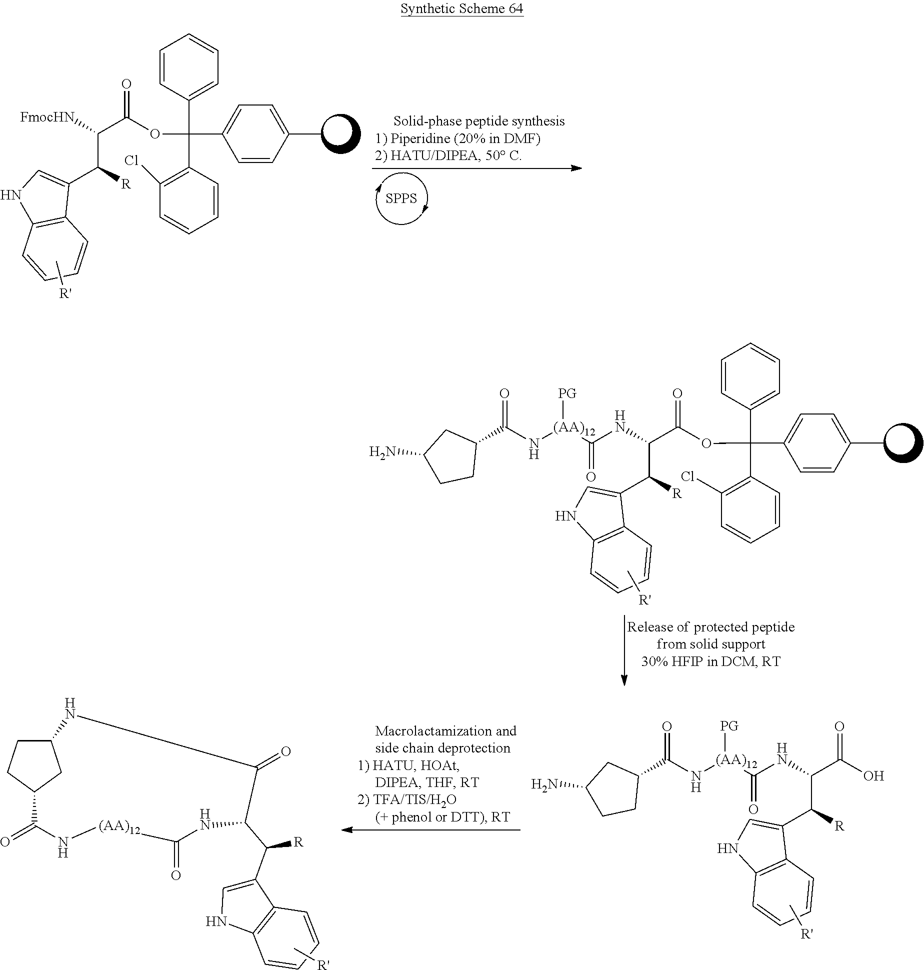

### 3. Procedure G

### a) Solid Phase Synthesis of Peptides (SPPS)

Peptides were synthesized on a Liberty Blue™ synthesizer from CEM Corporation, using standard solid-phase synthesis using Fmoc/tBu chemistry as summarized above in Synthetic Scheme 64.

Reactions were typically performed at a 100 μmol scale using 2-Chlorotrityl chloride resin (100-200 mesh, 1% DVB, 1.71 mmol/g loading, Novabiochem®) or 2-Chorotrityl chloride resin preloaded with the starting amino acid (0.66 mmol/g loading, Novabiochem®), based on the synthetic strategy.

The loading of the initial C-terminal amino acid to the 2CTC resin was performed in a fritted plastic syringe by treating with 1.0 millimolar excess of Fmoc-protected amino acid solution (0.2 M in a 4:1 (v/v) DCM/NMP mixture) and 5-fold molar excess of DIPEA at RT for 45 min. MeOH (0.4 mL) was added and the slurry was shaken for 15 min at RT. The resin was drained and washed with DMF. The Fmoc-protecting group was removed by treating with piperidine (20% (v/v) in DMF) at RT for 10 min. This process was repeated once more (twice total). The resin was washed with DMF, MeOH and DCM and dried under vacuum.

Every synthesis cycle included: (1) Fmoc amino acid deprotection by 20% (v/v) piperidine in anh. DMF (50° C. microwave assisted heating, 10 min; repeated twice); (2) Coupling (potentially repeated twice for difficult couplings) with Fmoc-protected amino acid/HATU/DIPEA (5, 5, and 10 equiv, respectively; 50° C. microwave assisted heating, 10 min). Cycles of Fmoc deprotection and Fmoc-protected amino acid coupling were repeated with the desired monomers until the full linear protected peptide was formed; (3) The peptide N-terminus was deprotected using 20% (v/v) piperidine in DMF (50° C. microwave assisted heating, 10 min; repeated twice).

### b) Cleavage of Protected Linear Peptide

After completion of the synthesis, the linear resin-bound peptide was transferred into a fritted plastic column. The resin was washed with DCM then the linear peptide was cleaved off the solid support by treatment with HFIP/DCM (3:7; v/v; 5 mL) at RT for 5 min. The solution was collected into a round bottom flask. This process was repeated twice more (three times total), and the cleavage solution was combined. The solvents were removed under reduced pressure.

### c) Solution Phase Macrolactamization

The crude protected linear peptide was resuspended in anh. THF (1.5 mg/mL). Anhydrous DMF (500 μL) was added. DIPEA (5 equiv), HOAt (0.8 equiv) and HATU (1.2 equiv) were added to the peptide solution and the solution stirred at RT. The reaction was monitored by UPLC-MS and stopped after complete conversion of the starting material. The reaction solution was then concentrated under reduced pressure to dryness.

### d) Deprotection of the Protected Cyclic Peptide

The cyclic peptide was deprotected by treatment with TFA/H₂O/TIS/Phenol (90:2:4:4; v/v/v/w; 15 mL) at RT for 1 h. The crude cyclic peptide was precipitated from the TFA cleavage solution using chilled MTBE (50 mL) and collected by centrifugation. The supernatant was removed. Additional cold MTBE (40 mL) was added to the peptide pellet and clarified by centrifugation. This process was repeated once more (twice total). The resulting crude peptide pellet was dissolved in 1:1 (v/v) mixture of DI water/MeCN (10 mL) and 5% (v/v) of TFA was added to the solution to remove the tryptophan adduct. The solution was frozen and lyophilized.

### e) HPLC Purification

The crude residue was then dissolved in DMSO and purified by preparative reversed-phase high performance liquid chromatography on a Waters™ Xbridge Protein BEH C4 OBD prep column (300 Å, 5 μm, column size 30×250 mm) using a Waters™ 2545 HPLC system equipped with Waters™ 2489 UV/Visible detector. Mobile phase: (A) 0.1% TFA in HPLC-grade water and (B) 0.1% TFA in HPLC-grade acetonitrile; flow rate: 50 mL/min; UV wavelength λ=214 nm; gradient: 0.5% B/min starting at 5% B less than the calculated % B required for compound elution over 20 min. UV absorbing fractions containing the target were collected and the fractions containing the desired product were combined, concentrated in vacuo and freeze-dried to afford the cyclized peptide as a solid.

Confirmation of identity and purity assessment of final compounds were performed by UPLC-MS, which was measured by a Rp Waters™ ACQUITY UPLC-MS system. Column: Waters™ ACQUITY UPLC Protein BEH C4 (300 Å, 1.7 μm, column size 2.1×100 mm, Milford, MA). Mobile phase: (A) 0.1% TFA in HPLC-grade water and (B) 0.1% TFA in HPLC-grade acetonitrile; injection volume: 0.5 μL; flow rate: 0.4 mL/min; Column Temperature: 45° C.; UV wavelength λ=214 nm; gradient: 20-20% B in 1 min and 20-40% in 3 min.

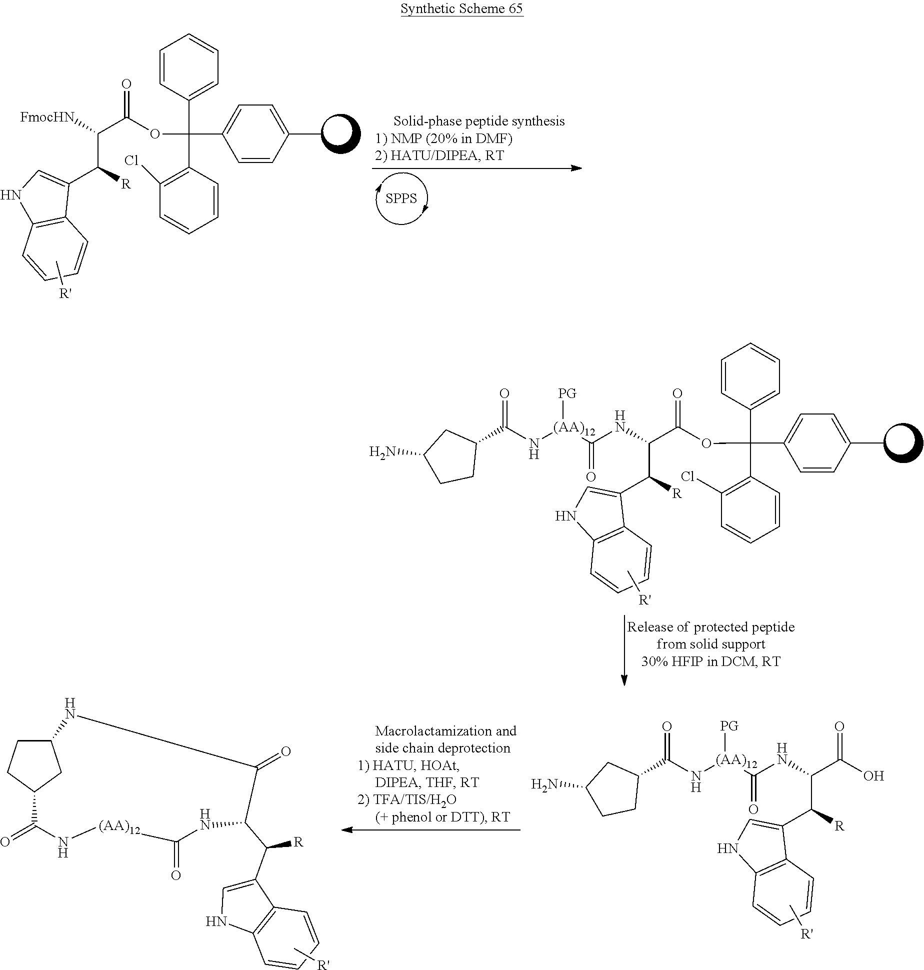

### 4. Procedure H

### a) Solid Phase Synthesis of Peptides (SPPS)

Peptides were synthesized on a Biotage® Syro II peptide synthesizer using standard solid-phase synthesis using Fmoc/tBu chemistry as summarized above in Synthetic Scheme 65.

Reactions were typically performed at the 25 μmol scale using 2-Chlorotrityl chloride resin (100-200 mesh, 1.6 mmol/g loading, Novabiochem®). Solutions of the reagents and amino acid monomers were prepared as follows: Fmoc-protected amino acids (0.2 M in DMF, NMP, or DMSO), HATU (0.5 M in DMF), DIPEA (2.0 M in NMP), 4-methylpiperidine (20% (v/v) in DMF).

The loading of the initial C-terminal amino acid to the 2CTC resin was performed as follows: 1.0 g of 2CTC resin (1.6 mmol/g loading, Novabiochem®) was swelled in DCM (10 mL) for 5 min. Meanwhile, Fmoc amino acid (1 mmol, 0.65 equiv) was dissolved in anh. DCM 8.7 mL). To that solution were added DIPEA (4 mmol, 4 equiv excess relative to AA equiv) and DMF (0.25 mL). The resulting suspension was vortexed until fully dissolved then transferred to the vessel containing the swelled resin and shaken at RT for 3 h. The resin was filtered, treated with of DCM/MeOH/DIPEA (17:2:1; v/v/v; 6 mL) at RT for 5 min (repeated three times), washed with DMF (3×10 mL), DCM (3×10 mL) then dry under vacuo overnight.

Every synthesis cycle included: (1) Double coupling with Fmoc-protected amino acid/HATU/DIPEA (4, 4 and 8 equiv, respectively; RT; 15 min). After the coupling reaction was complete the mixture was filtered, and the peptidyl resin was washed with DMF; (2) Fmoc deprotection (repeated three times) using 20% (v/v) 4-methylpiperidine in anh. DMF (RT; 3 min). The mixture was filtered, and the peptidyl resin was washed with anh. DMF. Cycles of Fmoc deprotection and Fmoc-protected amino acid coupling were repeated with the desired monomers until the full linear peptide was formed. (3) The peptide N-terminus was deprotected using 20% (v/v) 4-methylpiperidine in anh. DMF (RT; 3 min) and the peptidyl resin was washed with DCM.

### b) Cleavage of Protected Linear Peptide

To cleave the crude linear peptide precursor from the solid support, 1 mL of a mixture of HFIP/DCM (3:7; v/v) was added to the peptidyl resin and shaken for 5 min. The cleavage solution transferred to a 50 mL falcon and this process was repeated twice more (three times total) and the cleavage solution was added to the same tube. The solvent was removed under reduced pressure.

### c) Solution Phase Macrolactamization

The crude peptide was resuspended in 14 mL of THF. To that solution was added HOAt (2.0 mg dissolved in 70 μL of anh. DMF), HATU (8.4 mg dissolved in 70 μL of anh. DMF), and 8.4 μL of DIPEA. The reaction was shaken at RT for 20 min then checked by LC-MS. The reaction solution was then concentrated to dryness under reduced pressure.

### d) Deprotection of the Protected Cyclic Peptide

A solution of TFA/H₂O/TIS (95/2.5/2.5; v/v/v, 1 mL) was added to the crude protected cyclic peptide. The mixture was stirred for 10 min at RT. Cold Et₂O (15 mL) was added to the solution. The peptide was precipitated by centrifugation. The precipitate was washed with Et₂O (2×10 mL) and dried under vacuum o.n. to give the crude deprotected cyclic peptide as a solid.

### e) HPLC Purification

Purification was performed by preparative reversed-phase high performance liquid chromatography (Rp-HPLC) on Waters™ X-Bridge Prep C18 OBD Prep column (130 Å, 5 μm, column size 19×100 mm, Milford, MA) using a Waters™ MS-Directed AutoPurification HPLC-MS system. Mobile phase: (A) 0.16% TFA in HPLC-grade water and (B) 0.16% TFA in HPLC-grade acetonitrile; flow rate: 25 mL/min; UV wavelength λ=215 nm; gradient: 25-50% B over 5 min. Alternatively purification was performed on Waters™ CSH-C18 Column (19×250 mm, 5 μm) using an Agilent, with 1290 infinity II preparative LC system and LC-MSD XT mass spectrometer. Mobile phase: (A) 0.1% formic acid in HPLC-grade water and (B) 0.1% formic acid in HPLC-grade acetonitrile; flow rate: 25 mL/min; UV wavelength λ=215 nm; gradient: 20% B over 2.5 min, then increasing to 55% B over the next 17.5 min. UV absorbing fractions containing the target m/z ions were collected and the fractions containing product were confirmed by LC-MS.

Confirmation of identity and purity assessment of final compounds were performed by UPLC-MS, which was measured by a Rp Waters™ ACQUITY UPLC-MS system. Column: Waters™ XSelect CSH C18 Column (130 Å, 2.5 μm, column size 2.1×50 mm). Mobile phase: (A) 0.05% TFA in HPLC-grade water and (B) 0.05% TFA in HPLC-grade acetonitrile; injection volume: 1 μL; flow rate: 1 mL/min; UV wavelength λ=215 nm; gradient: 5-100% B in 5 min. Lyophilization of combined fractions containing pure peptide resulted in the final cyclized product as a powder.

D. Synthetic Procedures used to Prepare Modified Cyclic Peptides

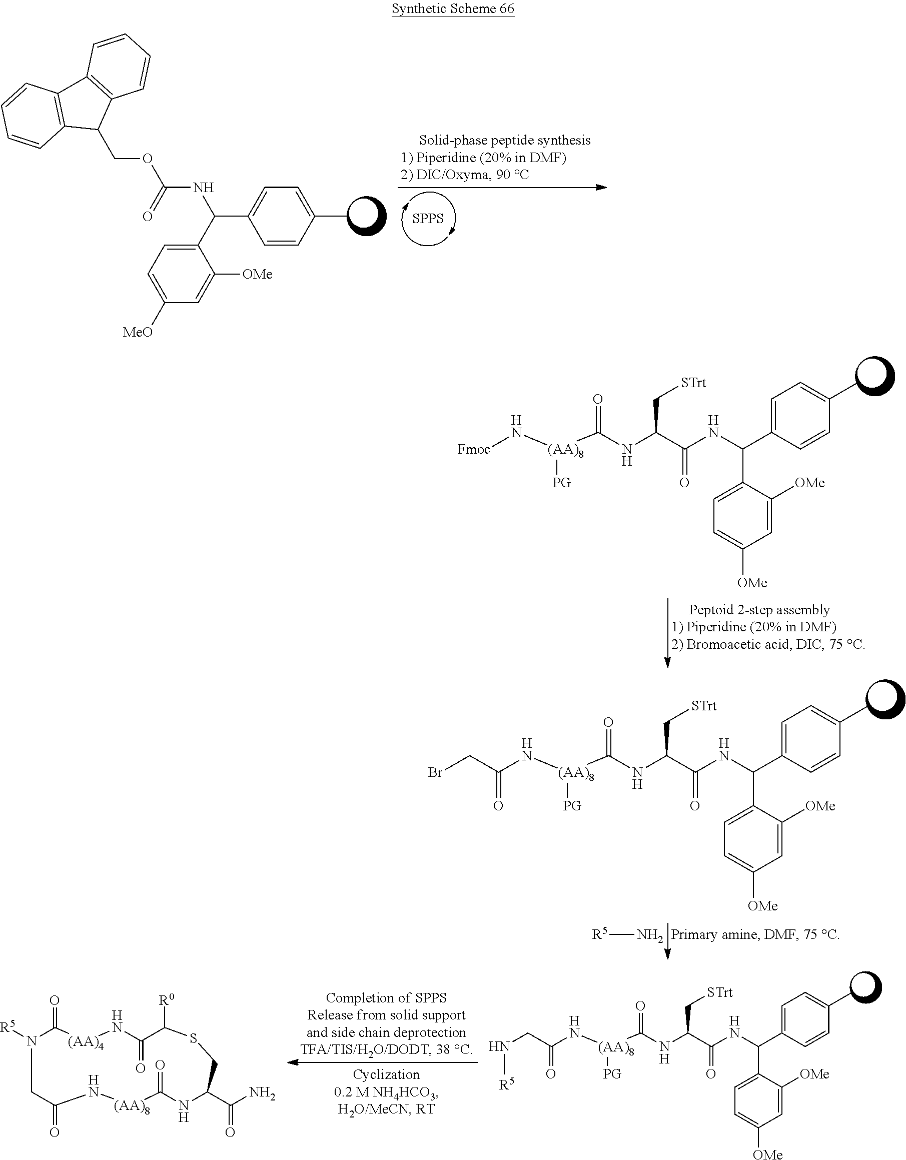

### 1. Procedure I

### a) Solid Phase Synthesis of Peptides (SPPS)

Peptoid-peptide hybrids containing building block peptoids (N-alkylated glycines), including but not limited to NxG27, NxG29, NxG38, NxG41, NxG42, and NetCNG, were synthesized on a Liberty Blue™ synthesizer from CEM Corporation, using standard solid-phase synthesis using Fmoc/tBu chemistry as summarized above in Synthetic Scheme 66.

Reactions were typically performed at a 50 μmol scale using Rink Amide MBHA LL resin (100-200 mesh, 0.4 mmol/g loading, 1% DVB cross-linked polystyrene, Novabiochem®). All the amino acids were dissolved at a 0.2 M concentration in anh. DMF or NMP. The amino acids were activated with equimolar amounts of Oxyma Pure solution (0.5 M in anh. DMF), and a 2-fold molar excess of DIC solution (0.5 M in anh. DMF).

Every synthesis cycle included: (1) Fmoc amino acid deprotection by 20% (v/v) piperidine in anh. DMF (90° C. microwave assisted heating, 1 min); (2) Coupling (potentially repeated twice for difficult couplings) with Fmoc-protected amino acid/DIC/Oxyma (5, 5, and 10 equiv, respectively; 90° C. microwave assisted heating, 2 min or 4 min). Cycles of Fmoc deprotection and Fmoc-protected amino acid coupling were repeated with the desired monomers until the full linear peptide was formed; (3) The assembly of the N-substituted glycine monomer on the resin as describes in the subsequent paragraph; (4) The peptide N-terminus was eventually capped with the required 2-chloroacetyl by coupling with chloroacetic acid and DIC (5 and 10 equiv, respectively; 90° C. microwave assisted heating, 2 min; repeated twice).

The peptoid monomer was directly assembled on resin through a “sub-monomer” process consisting of two steps: an acylation step using bromoacetic acid and DIC followed by reaction with a primary amine via nucleophilic displacement of the N-terminal bromide. This cycle included: (1) Fmoc amino acid deprotection by 20% (v/v) piperidine in anh. DMF (90° C. microwave assisted heating, 1 min); (2) Acylation with freshly recrystallized bromoacetic acid (0.2 M in anh. DMF) and DIC (0.5 M in anh. DMF) (5 and 10 equiv, respectively; 75° C. microwave assisted heating, 5 min; repeated twice); (3) Nucleophilic substitution of the terminal bromide with the desired primary amine (1.0 M in anh. DMF) (50 equiv; 75° C. microwave assisted heating, 10 min; repeated twice).

### b) Cleavage and Deprotection

After completion of the synthesis, the linear resin-bound peptide was transferred into a fritted plastic column. The resin was washed with DCM then dried under negative pressure on a Razor® peptide cleavage system from CEM Corporation. The peptide was deprotected and cleaved from the solid support by treatment with TFA/H₂O/TIS/DODT (92.5:2.5:2.5:2.5; v/v/v/v; 10 mL) at 38° C. for 30 min. The solution was collected into a 50 mL centrifuge tube. The resin washed with TFA (2×2 mL). The cleavage solution was partially concentrated to a volume of −5 mL under vacuum. The crude linear peptide was precipitated from the TFA cleavage solution using chilled MTBE (40 mL) and collected by centrifugation (4000 rpm). The supernatant was removed. Additional cold MTBE (30 mL) was added to the peptide pellet and clarified by centrifugation. The resulting crude peptide pellet was blown dried by a stream of nitrogen gas.

### c) Peptide Cyclization

The crude solids were dissolved in 1:1 (v/v) mixture of DI water/MeCN (15 mL). The pH was adjusted to pH 8 by addition of an aq. 0.2 M ammonium bicarbonate solution (3 mL). The solution was shaken at RT for approximately 2 h and monitored by UPLC-MS. After the reaction was complete, the reaction solution was quenched by addition of TFA (200 μL), frozen and lyophilized.

### d) HPLC Purification

The crude residue was then dissolved in DMSO and purified by preparative reversed-phase high performance liquid chromatography on a Waters™ SunFire Prep C18 OBD column (100 Å, 5 μm, column size 19×150 mm, Milford, MA) using an Agilent MS-Directed Preparative HPLC-MS system. Mobile phase: (A) 0.1% TFA in HPLC-grade water and (B) 0.1% TFA in HPLC-grade acetonitrile; flow rate: 35 m/min; UV wavelength λ=215 nm; gradient: 0.5% B/min for 40 min starting at 5% B less than the calculated % B required for compound elution. In some instances, better separation was obtained by 0.1% NH₄OH as modifier.

UV absorbing fractions containing the target m/z ions were collected and the fractions containing the desired product were combined, concentrated in vacuo and freeze-dried to afford the cyclized peptide as a solid.

Confirmation of identity and purity assessment of final compounds were performed by UPLC-MS, which was measured by a Rp Waters™ ACQUITY UPLC-MS system. Column: Waters™ Cortecs C18+ Column (90 Å, 1.6 μm, column size 2.1×100 mm, Milford, MA). Mobile phase: (A) 0.1% TFA in HPLC-grade water and (B) 0.1% TFA in HPLC-grade acetonitrile; injection volume: 0.5 μL; flow rate: 0.7 mL/min; Column Temperature: 60° C.; UV wavelength λ=215 nm; gradient: 2-35% in 9.1 min.

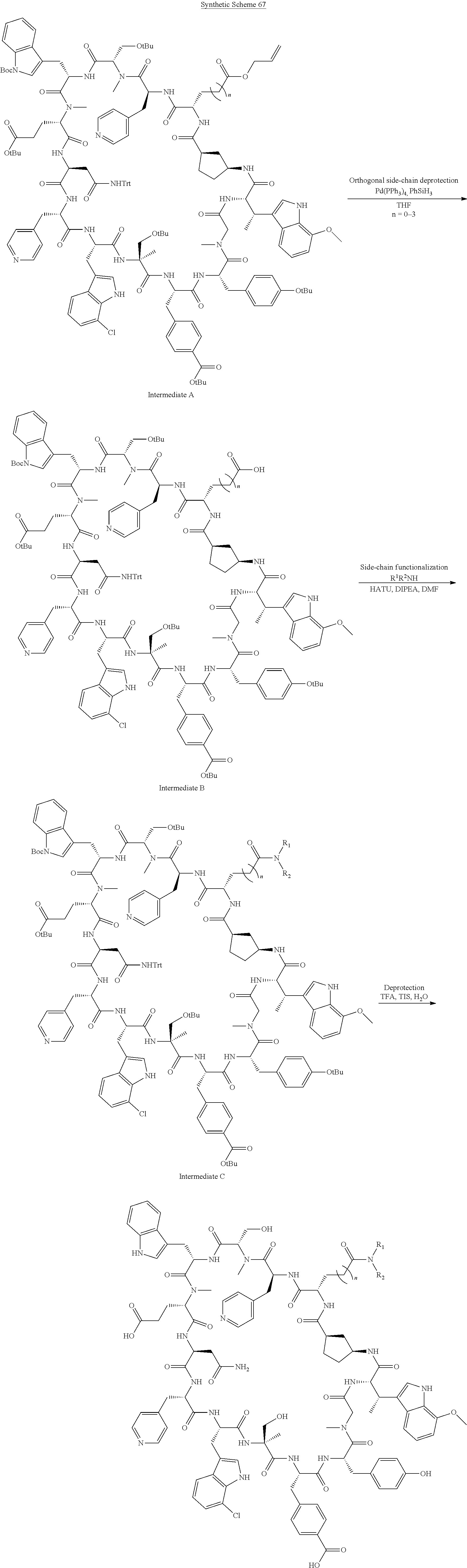

### 2. Procedure J

### a) Solid Phase Synthesis of Peptides (SPPS)

Peptides were synthesized on a Symphony® X synthesizer from Gyros Protein Technologies (Uppsala, Sweden), using standard solid-phase synthesis using Fmoc/tBu chemistry and Fmoc-L-glutamic acid γ-allyl ester (or related analogs including Fmoc-L-Aspartic acid β-allyl ester and Fmoc-L-homoGlutamic acid δ-allyl ester), where the allyl side chain can be orthogonally deprotected in the presence of acid-labile labile protecting groups for the selective late-stage functionalization of the side chain, as summarized above in Synthetic Scheme 67.

Reactions were typically performed using 2-Chlorotrityl chloride resin (100-200 mesh, 1.71 mmol/g loading, Novabiochem®). All the amino acids were dissolved at a 0.2 M concentration in anh. DMF or NMP. The amino acids were activated with equimolar amounts of HATU solution (0.5 M in anh. DMF) and a 2-fold molar excess of NMM solution (1.0 M in anh. DMF).

The loading of the initial C-terminal amino acid to the 2CTC resin was performed as follows: 2 mmol of 2CTC resin (1.17 g) was treated with DIPEA (1.68 mL, 9.6 mmol) and Fmoc-SbMeW70Me-OH (1.13 g, 2.4 mmol) in DCM (30 mL). The slurry was shaken at RT for 3 h, then filtered. The resin was washed with a mixture of DCM/MeOH/DIPEA (17:2:1; v/v/v; 3×30 mL), followed by DMF (3×30 mL) and DCM (3×30 mL), dried under vacuum.

Every synthesis cycle included: (1) Fmoc amino acid deprotection by 20% (v/v) piperidine in anh. DMF (RT; 3×5 min); (2) Double coupling with Fmoc-protected amino acid/HATU/NMM (4, 4, and 8 equiv, respectively; RT; 2×70 min). Cycles of Fmoc deprotection and Fmoc-protected amino acid coupling were repeated with the desired monomers until the full linear peptide was formed. Cycles of Fmoc deprotection and Fmoc-protected amino acid coupling were repeated with the desired monomers until the full linear peptide was formed; (3) The peptide N-terminus was deprotected using 20% (v/v) piperidine in anh. DMF (RT; 3×5 min).

### b) Cleavage of Protected Linear Peptide

To cleave the crude linear peptide precursor from the solid support, a 1:3 (v/v) mixture of HFIP/DCM (40 mL) was added to the peptidyl resin and shaken for 30 min. The cleavage solution was filtered and transferred in a centrifuge tube. This process was repeated twice more (three times total). the combined cleavage solution was concentrated under reduced pressure to give the crude protected linear peptide (2.60 g) and used in the next step directly without any further purification. MS ESI calculated for C₁₄₉H₁₈₅ClN₂₀O₂₈ [M+2H]²+1370.17, found 1371.1.

### c) Solution Phase Macrolactamization

To a stirred solution of crude linear peptide (2.60 g, 0.95 mmol) in anh. DMF (100 mL) and DCM (1700 mL) was added HATU (0.30 g, 0.79 mmol), HOAT (0.13 g, 0.95 mmol), and DIPEA (1.32 mL, 7.59 mmol). The resulting solution was stirred at RT. After 30 min, additional HATU (0.10 g, 0.26 mmol) was added, and the reaction mixture was stirred at RT for 45 min. The volatile was removed under reduced pressure. The residue was diluted in EtOAc (500 mL) and washed with NaHCO₃/brine solution (1:1; v/v; 3×300 mL), dried over anh. Na₂SO₄, filtered and concentrated in vacuo. The residue was purified on silica gel column chromatography, eluting with MeOH 0-15% in DCM to afford macrolactam Intermediate A as a solid. MS ESI calculated for C₁₄₉H₁₈₃ClN₂₀O₂₇ [M+2H]²⁺ 1361.17, found 1361.9.

### d) Orthogonal Side-Chain Deprotection

A stirred solution of Intermediate A (555 mg, 0.204 mmol) in anh. THF (30 mL) was degassed with nitrogen for 30 min. Phenylsilane (50 μL, 0.408 mmol) and tetrakis(triphenylphosphine)palladium(0) (12 mg, 10.2 μmol) was added and the resulting mixture was stirred at RT under nitrogen for 3.5 h. The mixture was concentrated under reduced pressure, and the residue was purified on a silica gel column chromatography, eluting with MeOH 0-20% in DCM to afford macrolactam Intermediate B as a solid. MS ESI calculated for C₁₄₆H₁₇₉ClN₂₀O₂₇ [M+2H]²+1341.15, found 1341.8.

### e) Side-Chain Functionalization

To a stirred solution of Intermediate B (15.0 mg, 5.6 μmol) in anh. DMF (1 mL) and desired primary of secondary amine R¹R²NH (such as, but not limited to, 3-aminopyridine) (8.4 μmol) was added HATU (3.2 mg, 8.4 μmol) and DIPEA (6 μL, 34 μmol). The resulting solution was stirred at RT for 2 h. The solution was then concentrated under high vacuum. The crude product Intermediate C was used in the next step directly without any further purification.

### f) Deprotection of the Protected Cyclic Peptide

A solution of TFA/H₂O/TIS (92.5/5/2.5; v/v/v; 2 mL) was added to the crude protected cyclic peptide Intermediate C. The mixture was stirred at RT for 90 min. Cold Et₂O (35 mL) was added to the solution and the precipitated peptide was collected by centrifugation. The precipitate was washed with Et₂O (2×30 mL) and dried under vacuum o.n. to give the crude deprotected cyclic peptide as a solid.

### g) HPLC Purification

Purification was performed by preparative reversed-phase high performance liquid chromatography (Rp-HPLC) on Waters™ X-Bridge Prep C18 OBD Prep column (130 Å, 5 μm, column size 19×100 mm, Milford, MA) using a Waters™ MS-Directed AutoPurification HPLC-MS system. Mobile phase: (A) 0.16% TFA in HPLC-grade water and (B) 0.16% TFA in HPLC-grade acetonitrile; flow rate: 25 mL/min; UV wavelength λ=215 nm; gradient: 25-50% B over 5 min. Alternatively purification was performed on Waters™ CSH-C18 Column (19×250 mm, 5 μm) using an Agilent, with 1290 infinity II preparative LC system and LC-MSD XT mass spectrometer. Mobile phase: (A) 0.1% formic acid in HPLC-grade water and (B) 0.1% formic acid in HPLC-grade acetonitrile; flow rate: 25 mL/min; UV wavelength λ=215 nm; gradient: 20% B over 2.5 min, then increasing to 55% B over the next 17.5 min. UV absorbing fractions containing the target m/z ions were collected and the fractions containing product were confirmed by LC-MS.

Confirmation of identity and purity assessment of final compounds were performed by UPLC-MS, which was measured by a Rp Waters™ ACQUITY UPLC-MS system. Column: Waters™ XSelect CSH C18 Column (130 Å, 2.5 μm, column size 2.1×50 mm). Mobile phase: (A) 0.05% TFA in HPLC-grade water and (B) 0.05% TFA in HPLC-grade acetonitrile; injection volume: 1 μL; flow rate: 1 mL/min; UV wavelength λ=215 nm; gradient: 5-100% B in 5 min. Lyophilization of combined fractions containing pure peptide resulted in the final cyclized product SEQ ID NO: 166 as a powder. MS ESI calculated for C₁₀₇H₁₂₁ClN₂₂O₂₄ [M+2H]²⁺ 1067.94, found 1068.19.

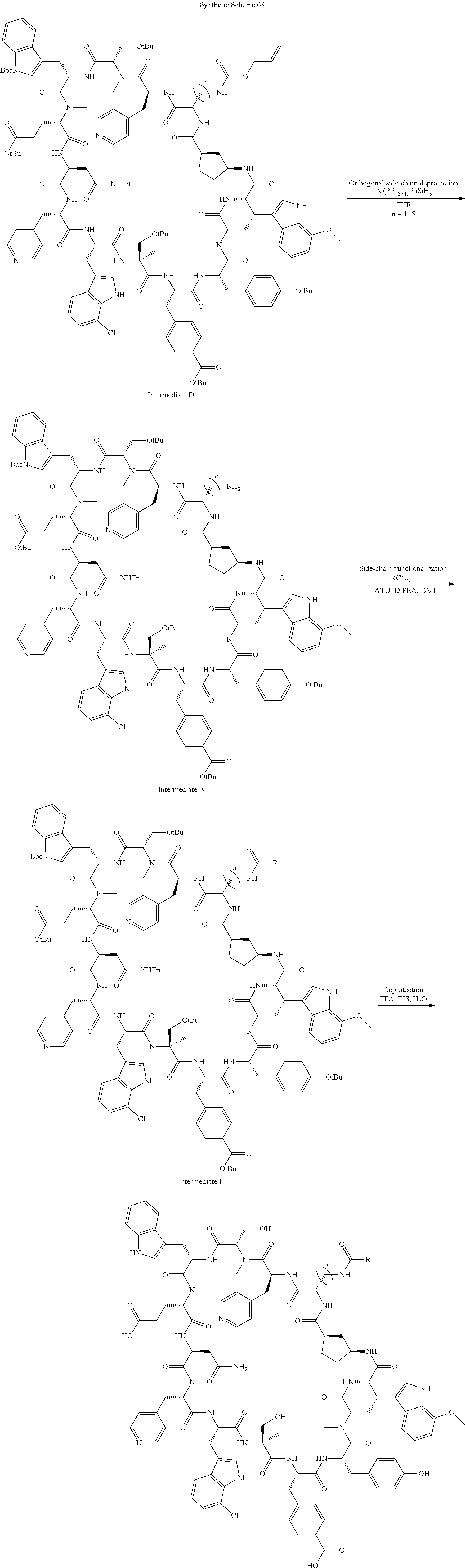

### 3. Procedure K

### a) Solid Phase Synthesis of Peptides (SPPS)

Peptides were synthesized on a Symphony® X synthesizer from Gyros Protein Technologies, using standard solid-phase synthesis using Fmoc/tBu chemistry and N_{α}-Fmoc-N_{γ}-Alloc-L-2,4-diaminobutyric acid or Fmoc-Dab(Alloc)-OH (or related analogs including Fmoc-Dap(Alloc)-OH, Fmoc-Orn(Alloc)-OH, Fmoc-Lys(Alloc)-OH, Fmoc-homoLys(Alloc)-OH), where the allyl side chain can be orthogonally deprotected in the presence of acid-labile labile protecting groups for the selective late-stage functionalization of the side chain, as summarized above in Synthetic Scheme 68.

Reactions were typically performed using 2-Chlorotrityl chloride resin (100-200 mesh, 1.71 mmol/g loading, Novabiochem®). All the amino acids were dissolved at a 0.2 M concentration in anh. DMF or NMP. The amino acids were activated with equimolar amounts of HATU solution (0.5 M in anh. DMF) and a 2-fold molar excess of NMM solution (1.0 M in anh. DMF).

The loading of the initial C-terminal amino acid to the 2CTC resin was performed as follows: 2 mmol of 2CTC resin (1.17 g) was treated with DIPEA (1.68 mL, 9.6 mmol) and Fmoc-SbMeW70Me-OH (1.13 g, 2.4 mmol) in DCM (30 mL). The slurry was shaken at RT for 3 h, then filtered. The resin was washed with a mixture of DCM/MeOH/DIPEA (17:2:1; v/v/v; 3×30 mL), followed by DMF (3×30 mL) and DCM (3×30 mL), dried under vacuum.

Every synthesis cycle included: (1) Fmoc amino acid deprotection by 20% (v/v) piperidine in anh. DMF (RT; 3×5 min); (2) Double coupling with Fmoc-protected amino acid/HATU/NMM (4, 4, and 8 equiv, respectively; RT; 2×70 min). Cycles of Fmoc deprotection and Fmoc-protected amino acid coupling were repeated with the desired monomers until the full linear peptide was formed. Cycles of Fmoc deprotection and Fmoc-protected amino acid coupling were repeated with the desired monomers until the full linear peptide was formed; (3) The peptide N-terminus was deprotected using 20% (v/v) piperidine in anh. DMF (RT; 3×5 min).

### b) Cleavage of Protected Linear Peptide

To cleave the crude linear peptide precursor from the solid support, a 1:3 (v/v) mixture of HFIP/DCM (40 mL) was added to the peptidyl resin and shaken for 30 min. The cleavage solution was filtered and transferred in a centrifuge tube. This process was repeated twice more (three times total). The combined cleavage solution was concentrated under reduced pressure and used in the next step directly without any further purification.

### c) Solution Phase Macrolactamization

To a stirred solution of crude linear peptide (2.60 g, 0.95 mmol) in anh. DMF (100 mL) and DCM (1700 mL) was added HATU (0.30 g, 0.79 mmol), HOAT (0.13 g, 0.95 mmol), and DIPEA (1.32 mL, 7.59 mmol). The resulting solution was stirred at RT. After 30 min, additional HATU (0.10 g, 0.26 mmol) was added, and the reaction mixture was stirred at RT for 45 min. The volatile was removed under reduced pressure. The residue was diluted in EtOAc (500 mL) and washed with NaHCO₃/brine solution (1:1; v/v; 3×300 mL), dried over anh. Na₂SO₄, filtered and concentrated in vacuo. The residue was purified on silica gel column chromatography, eluting with MeOH 0-15% in DCM to afford macrolactam Intermediate D as a solid. MS ESI calculated for C₁₄₉H₁₈₄ClN₂₁O₂₇ [M+2H]²⁺ 1368.67, found 1369.0.

### d) Orthogonal Side-Chain Deprotection

A stirred solution of Intermediate D (436 mg, 0.159 mmol) in anh. THF (10 mL) was degassed with nitrogen for 30 min. Phenylsilane (40 μL, 0.32 mmol) and tetrakis(triphenylphosphine)palladium(0) (9.2 mg, 7.8 μmol) was added and the resulting mixture was stirred at RT under N₂ for 4 h. The mixture was concentrated under reduced pressure, and the residue was purified on a silica gel column chromatography, eluting with MeOH 0-40% in DCM to afford macrolactam Intermediate E as a solid. MS ESI calculated for C₁₄₅H₁₈₀ClN₂₁O₂ [M+2H]²⁺ 1326.66, found 1327.3.

### e) Side-Chain Functionalization

To a stirred solution of Intermediate E (15.0 mg, 5.6 μmol) in anh. DMF (1 mL) and desired primary of secondary amine RCO₂H (such as, but not limited to, isoxazole-3-carboxylic acid) (11 μmol) was added HATU (4.3 mg, 11 μmol) and DIPEA (10 μL, 57 μmol). The resulting solution was stirred at RT for 1 h. The solution was then concentrated under high vacuum. The crude product Intermediate F was used in the next step directly without any further purification.

### f) Deprotection of the Protected Cyclic Peptide

A solution of TFA/H₂O/TIS (92.5/5/2.5; v/v/v; 2 mL) was added to the crude protected cyclic peptide Intermediate F. The mixture was stirred at RT for 90 min. Cold Et₂O (35 mL) was added to the solution and the precipitated peptide was collected by centrifugation. The precipitate was washed with Et₂O (2×30 mL) and dried under vacuum o.n. to give the crude deprotected cyclic peptide as a solid.

### g) HPLC Purification

Purification was performed by preparative reversed-phase high performance liquid chromatography (Rp-HPLC) on Waters™ X-Bridge Prep C18 OBD Prep column (130 Å, 5 μm, column size 19×100 mm, Milford, MA) using a Waters™ MS-Directed AutoPurification HPLC-MS system. Mobile phase: (A) 0.16% TFA in HPLC-grade water and (B) 0.16% TFA in HPLC-grade acetonitrile; flow rate: 25 mL/min; UV wavelength λ=215 nm; gradient: 25-50% B over 5 min. Alternatively purification was performed on Waters™ CSH-C18 Column (19×250 mm, 5 μm) using an Agilent, with 1290 infinity II preparative LC system and LC-MSD XT mass spectrometer. Mobile phase: (A) 0.1% formic acid in HPLC-grade water and (B) 0.1% formic acid in HPLC-grade acetonitrile; flow rate: 25 mL/min; UV wavelength λ=215 nm; gradient: 20% B over 2.5 min, then increasing to 55% B over the next 17.5 min. UV absorbing fractions containing the target m/z ions were collected and the fractions containing product were confirmed by LC-MS.

Confirmation of identity and purity assessment of final compounds were performed by UPLC-MS, which was measured by a Rp Waters™ ACQUITY UPLC-MS system. Column: Waters™ XSelect CSH C18 Column (130 Å, 2.5 μm, column size 2.1×50 mm). Mobile phase: (A) 0.05% TFA in HPLC-grade water and (B) 0.05% TFA in HPLC-grade acetonitrile; injection volume: 1 μL; flow rate: 1 mL/min; UV wavelength λ=215 nm; gradient: 5-100% B in 5 min.

Lyophilization of combined fractions containing pure peptide resulted in the final cyclized product SEQ ID NO: 169 as a powder. MS ESI calculated for C₁₀₇H₁₂₁ClN₂₃O₂₄ [M+2H]²+1073.94, found 1074.7.

### E. Synthesis of Specific Compounds

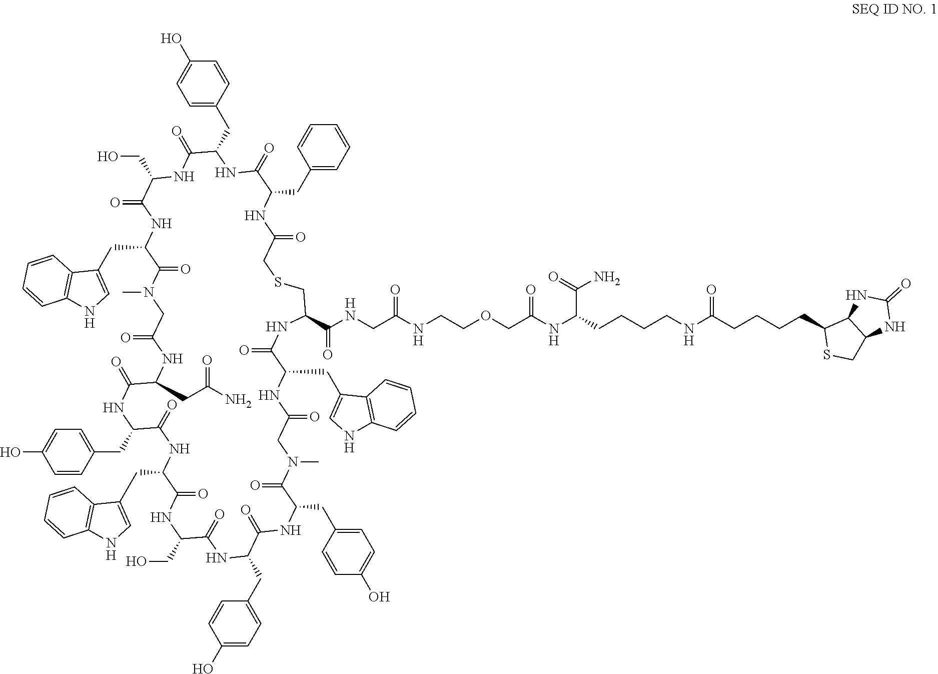 [SMILES: CN1CC(=O)N[C@@H](CC(N)=O)C(=O)N[C@@H](Cc2ccc(O)cc2)C(=O)N[C@@H](Cc2c[nH]c3ccccc23)C(=O)N[C@@H](CO)C(=O)N[C@@H](Cc2ccc(O)cc2)C(=O)N[C@@H](Cc2ccc(O)cc2)C(=O)N(C)CC(=O)N[C@@H](Cc2c[nH]c3ccccc23)C(=O)N[C@H](C(=O)NCC(=O)NCCOCC(=O)N[C@@H](CCCCNC(=O)CCCC[C@@H]2SC[C@@H]3NC(=O)N[C@@H]32)C(N)=O)CSCC(=O)N[C@@H](Cc2ccccc2)C(=O)N[C@@H](Cc2ccc(O)cc2)C(=O)N[C@@H](CO)C(=O)N[C@@H](Cc2c[nH]c3ccccc23)C1=O]

The TR-FRET probe (SEQ ID NO. 1) was prepared using Synthetic Procedure A starting with Fmoc-Lys(biotin)-OH (Novabiochem®, cat. #852097) and adding a Gly-Aea spacer attached to the carboxy group of the C-terminal cysteine. MS ESI calculated for C₁₂₁H₁₄₅ClN₂₅O₂₈S₂[M+2H]²⁺ 1231.51, found 1231.5.

### Biological Assays

A. Procedure for Human sTNFR1-Peptide Displacement Assay

The affinity was determined using LanthaScreen Eu-based time-resolved fluorescence resonance energy transfer (TR-FRET)-based binding assay.

A TR-FRET assay measuring the displacement of biotinylated peptide SEQ ID NO. 1 from human soluble TNFR1 (sTNFR1) in a pre-formed complex by a macrocyclic peptide yields the affinity (K_{d}^{app}) of the cyclic peptide for sTNFR1. The reagents for the assay include: the C′-terminally 6His-tagged sTNFR1 (residues 1-211) protein purified from a Sf21 baculovirus expression system, the biotin-tagged B0372 peptide described in section “Preparation of Final Compounds”, part E, the LanthaScreen™ anti-His Tag Europium donor (Life Technologies Corporation (San Diego, CA), cat. #PV5597), and a streptavidin-conjugate Alexa Fluor™ 647 acceptor (Invitrogen (Waltham, MA), cat. #S32357). Time-resolved TR-FRET is achieved when sTNFR1 is bound to biotinylated peptide SEQ ID NO. 1 through coupling of the donor and acceptor FRET pair.

100 nM biotinylated peptide SEQ ID NO. 1 and 0.25 nM sTNFR1-6His are added to a binding buffer (1×DPBS, Gibco™, cat. #20012-027 with 0.1% BSA and 0.05% Tween® 20) to form the protein-peptide complex. 100 nM biotinylated peptide SEQ ID NO. 1 was chosen as it reflects the ˜K_{d} for this complex. After 30 min at ambient temperature, each test peptide (2 mM stock in DMSO) is titrated in a 20 point, ˜3-fold discrete dose-response using an acoustic-dispense liquid handler. Specifically, 80 nL of peptide/DMSO is transferred into a ProxiPlate-384 Plus 384-well assay plate (PerkinElmer, cat. #6008289). To this plate, 8 μL of the previously formed sTNFR 1-biotinylated peptide SEQ ID NO. 1 complex is added and allowed to reach binding equilibrium with the test peptide at ambient temperature for 90 min. For TR-FRET detection, 8 μL of 0.25 nM of LanthaScreen™ anti-His Tag Europium and 25 nM of streptavidin-conjugate Alexa Fluor™ 647 in the same binding buffer are added to the assay plate and read on an EnVision® plate reader (PerkinElmer (Boston, MA)) after a 90 min final incubation.

The TR-FRET signal is measured with the following EnVision® settings: excitation laser at 337 nm; emission1=615 nm, emission2=665 nm, LANCE/DELFIA dichroic mirror, delay time=100 ms. The signal of each well is determined as the ratio of the emission at 665 nm to that at 615 nm. Percent displacement is determined by normalization to control wells containing DMSO (0%) or a saturating concentration of a displaceable peptide (100%). The %-effect as a function of peptide concentration is fit to a five-parameter logistic fit based on the Morrison equation to account for tight binding kinetics. The inflection point of the 5-parameter fit is the measured K_{d}^{app} reported in Table 2. It should be noted that peptides with weaker binding affinity where tight binding was not observed under these assay conditions were titrated with 10 pts titration range and dose-response curves were analyzed using a 4-parameter logistic model to calculate IC₅₀ values (Table 2).

STNFR1 sequence (SEQ. ID NO. P1):
LVPHLGDREKRDSVCPQGKYIHPQNNSICCTKCHKGTYLYNDCPGPGQD
TDCRECESGSFTASENHLRHCLSCSKCRKEMGQVEISSCTVDRDTVCGC
RKNQYRHYWSENLFQCFNCSLCLNGTVHLSCQEKQNTVCTCHAGFFLRE
NECVSCSNCKKSLECTKLCLPQIENVKGTEDSGTTGGGLNDIFEAQKIE
WHELEVLFQGPHHHHHHGSWSHPQFEK

B. Procedure for Binding Affinity and Selectivity measured by SPR

Affinity and selectivity for hTNFR1 and TNFR2 was determined using SPR. Human TNFR1 or TNFR2 genes (extracellular domain only) were synthesized with a C-terminal Avi-tag, HRV 3C protease cleavage site followed by a 6His-tag for affinity purification. The final gene product was cloned into pBAC™-1 vector (Millipore Sigma) for expression testing in insect cells. Constructs were transfected into Sf21 cells for baculovirus generation followed by protein expression analysis in Sf21 and Tni cell lines. Large scale expression in Sf21 cells was performed using baculovirus infected insect cells (BIICs) infected at a MOI=2.0 grown for 72 h. Target proteins secreted into the media were harvested via centrifugation and concentrated using diafiltration. TNFR was purified by immobilized metal ion affinity chromatography (IMAC) using a Ni-Sepharose HisTrap™ column (Cytiva™). Isolated TNFR had His tag removed using HRV 3C protease followed by additional purification using Ni-NTA chromatography. Biotinylation was performed on Ni-NTA flow-through fractions concentrated to 45 μM and treated with BirA ligase (1:100, μg/μg ratio) in 50 mM Tris pH 7.5, 150 mM NaCl, 50 μM biotin, 10 mM magnesium acetate, 10 mM ATP at 4° C. for 20 h. Final purification of biotin-TNFR and buffer exchange into 20 mM HEPES pH 7.5, 150 mM NaCl was performed using a HiLoad® Superdex® 200 26/60 column (Cytiva™). Highly purified protein was aliquoted and stored at −80° C.

Human TNFR1 construct sequence (SEQ. ID NO. P2):
MGLSTVPDLLLPLVLLELLVGIYPSGVIGLVPHLGDREKRDSVCPQGKY
IHPQNNSICCTKCHKGTYLYNDCPGPGQDTDCRECESGSFTASENHLRH
CLSCSKCRKEMGQVEISSCTVDRDTVCGCRKNQYRHYWSENLFQCFNCS
LCLNGTVHLSCQEKQNTVCTCHAGFFLRENECVSCSNCKKSLECTKLCL
PQIENVKGTEDSGTTGGGLNDIFEAQKIEWHELEVLFQGPHHHHHHGSW
SHPQFEK
Human TNFR2 construct sequence (SEQ. ID NO. P3):
MAPVAVWAALAVGLELWAAAHALPAQVAFTPYAPEPGSTCRLREYYDQT
AQMCCSKCSPGQHAKVFCTKTSDTVCDSCEDSTYTQLWNWVPECLSCGS
RCSSDQVETQACTREQNRICTCRPGWYCALSKQEGCRLCAPLRKCRPGF
GVARPGTETSDVVCKPCAPGTFSNTTSSTDICRPHQICNVVAIPGNASM
DAVCTSTSPTRSMAPGAVHLPQPVSTRSQHTQPTPEPSTAPSTSFLLPM
GPSPPAEGSTGDGGGLNDIFEAQKIEWHELEVLFQGPHHHHHHGSWSHP
QFEK

The SPR assay uses a Cytiva™ 8K or 8K+ biosensor instrument to measure the kinetics of peptide analytes binding to captured human TNFR1 extracellular domain (ECD) protein; peptide analytes are also evaluated for any off-target binding to human TNFR2 ECD protein in parallel. Recombinant human TNFR1 ECD and human TNFR2 ECD proteins, biotinylated via engineered C-terminal avi-tags, are immobilized to a regenerable streptavidin biosensor chip surface (Cytiva™ Biotin CAPture kit, Series S; cat. #28920234). Peptide analytes binding to immobilized human TNFR1 ECD protein and their subsequent dissociation are conducted in a buffer of 10 mM HEPES pH 7.4, 150 mM NaCl, 3 mM EDTA, 0.05% P20,2% DMSO under laminar flow conditions and measured in response units (RU), which are directly proportional to the accumulated mass on the surface of the biosensor chip; the biosensor chip surface is regenerated via Cytiva™ proprietary methods between the evaluation of each peptide analyte. The binding and dissociation of peptide analytes are displayed as sensorgrams in real time and recorded for subsequent determination of binding kinetics. The SPR assay determines the association and dissociation rate constants, kₒₙ and k_{off}, for each peptide analyte and, from these values, calculates the binding affinity, KA reported in Table 2.

C. Procedure for THP-1 Lucia NF-κB cells

The inhibition of TNFα-induced NF-κB signal transduction pathways was evaluated in THP-1 cells. THP-1 cells harboring NF-κB-inducible Lucia™ reporter gene (InvivoGen, cat. #thp1-nfkb) designed for monitoring NF-κB signal transduction pathway was employed to assess the inhibitory effect macrocyclic peptides on TNFR1 signaling. The cells were cultured in media containing RPMI 1640, 2 mM L-glutamine, 25 mM HEPES, 10% heat-inactivated fetal bovine serum with appropriate selection antibiotics. Assays were performed in media that was devoid of selection antibiotics. For profiling, 125 nL of peptides in DMSO (10 mM) were dispensed into Falcon® 384-well Optilux white/clear flat bottom TC-treated microtest microplate (Corning, cat. #353963) using Echo® 555. The peptides were tested in a dose response to determine EC50's [10 or 1 μM top concentration and 1:3 dilutions]. A suspension of 1 million cells/mL was dispensed at 20 μL/well in a 384-well plate to a density of 20,000 cells/well and pre-incubated with peptides at 37° C. for 1 h. Subsequently, 5 μL of TNF containing media (recombinant human TNFα; R&D Systems USA, cat. #210-TA-005/CF) or TNF-free media (for controls) was added to the wells. The final concentration of TNFα in the assay in 10 ng/mL. After o.n. incubation at 37° C. (16-18 h), 5 μL of supernatant was aspirated and transferred to a 384-well microplate (PerkinElmer, cat. #6007680) for measurement of secreted luciferase activity with 25 μL of QUANTI-Luc™ luminescence assay reagent (InvivoGen, cat. #rep-qlc1) in each well. The luminescence was measured in EnVision® Multimode Plate Reader (PerkinElmer). Dose response curves were analyzed using a 4-parameter logistic model to calculate IC₅₀ values, reported in Table 2, using TIBCO® Spotfire® software employing appropriate Minimum and Maximum controls.

The amino acid sequences, synthetic procedure used to synthesize and purify the macrocyclic peptides, calculated monoisotopic masses, molecular formulas, calculated molecular weights and mass spectral data of Example Nos. 1-289 (SEQ ID NOS. 1-289) are provided below in Table 1.

The biological activities (binding IC₅₀ and K_{d}^{app} in the displacement assay, affinity K_{D}, and cell activity in THP-1 cells) of Example Nos. 1-289 (SEQ ID NOS. 1-289) are provided below in Table 2.

TABLE 1
Selective Compounds of the Invention disclosed as
Examples 1-298 (SEQ ID NOS. 1-289)

| | | | | | | | Mea- | |
|---|---|---|---|---|---|---|---|---|
| SEQ | | Pro- | Molecular | Mole- | Exact | | sured | |
| ID | | ced- | Weight | cular | mass | Purity | Mass | Observed |
| NO. | Sequence | ure | (g/mol) | Formula | (Da) | (%) | (Da) | Ion |
| 1 | cyclo(CLAc-F-Y-S-W-Sar-N-Y-W-S-Y-Y-Sar- | A | 2461.72 | C121H | 2460.01 | 95.0 | 1231.5 | [M + 2H] |
| | W-C)-G-Aea-K(N-Biotin)-NH2 | | | 145N2 | | | | 2+ |
| | | | | 5O28S2 | | | | |
| 2 | cyclo(bhcLeu-Nle-4Pal-Pipt5OH- | E | 2219.43 | C111H | 2217.94 | 92.0 | 1110.2 | [M + 2H] |
| | TrpCH2COOH-dProt4NMe2-N-Phe4COOH- | | | 131N2 | | | | 2+ |
| | Trp7CN-aMeS-F4SO2NH2-Phe4CONH2-Sar- | | | 3O25S | | | | |
| | SbMeW7OMe) | | | | | | | |
| 3 | cyclo(bhcLeu-Nle-4Pal-Pipt5OH-W- | E | 2168.75 | C109H | 2166.89 | 100.0 | 1084.7 | [M + 2H] |
| | dProt4OH-N-TyrOcm-Trp7Cl-aMeS- | | | 127C1N | | | | 2+ |
| | Phe4CONH2-TyrOcm-Sar-SbMeW7OMe) | | | 20O26 | | | | |
| 4 | cyclo(bhcLeu-Nle-4Pal-Pipt5OH-Trp7az- | E | 2194.34 | C108H | 2192.87 | 100.0 | 1097.9 | [M + 2H] |
| | dProt40AcOH-N-Phe4COOH-Trp7CN-aMeS- | | | 124N2 | | | | 2+ |
| | F4SO2NH2-Phe4COOH-Sar-SbMeW7OMe) | | | 2O27S | | | | |
| 5 | cyclo(bhcLeu-Nle-4Pal-Pip2c-W-NMeE-N- | G | 2053.66 | C104H | 2051.88 | 99.4 | 1O27.6 | [M + 2H] |
| | 4Pal-Trp7Cl-aMeS-Phe4COOH-Y-Sar- | | | 122C1N | | | | 2+ |
| | SbMeW7OMe) | | | 21O22 | | | | |
| 6 | cyclo(bhcLeu-Nle-4Pal-Pipt5OH- | E | 2191.31 | C109H | 2189.93 | 100.0 | 1096.4 | [M + 2H] |
| | TrpCH2COOH-dProt4OH-N-TyrOcm- | | | 127N2 | | | | 2+ |
| | Trp7CN-aMeS-AlaPyrim4NH2-TyrOcm-Sar- | | | 3O27 | | | | |
| | SbMeW7OMe) | | | | | | | |
| 7 | cyclo(bhcLeu-Nle-4Pal-Pip2c-W-NMeE-N- | H | 2071.65 | C104H | 2069.87 | 97.8 | 1035.6 | [M + 2H] |
| | 4Pal-Trp7Cl-aMeS-Phe2F4COOH-Y-Sar- | | | 121CIF | | | | 2+ |
| | SbMeW7OMe) | | | N21O22 | | | | |
| 8 | cyclo(bhcLeu-Nle-4Pal-Pipt5OH-Trp7az- | F | 2118.24 | C107H | 2116.89 | 100.0 | 1059.7 | [M + 2H] |
| | dProt4OH-N-Phe3F4COOH-Trp7CN-aMeS- | | | 121FN | | | | 2+ |
| | Phe4CONH2-Phe4COOH-Sar-SbMeW7OMe) | | | 22O24 | | | | |
| 9 | cyclo(bhcLeu-Nle-4Pal-Pipt5OH- | F | 2234.47 | C112H | 2232.96 | 98.4 | 1117.3 | [M + 2H] |
| | TrpCH2COOH-dProt4NMe3-N-Phe4COOH- | | | 134N2 | | | | 2+ |
| | Trp7CN-aMeS-F4SO2NH2-Phe4CONH2-Sar- | | | 3O25S | | | | |
| | SbMeW7OMe) | | | | | | | |
| 10 | cyclo(bhcLeu-Ahp-4Pal-Pip2c-W- | H | 2112.30 | C108H | 2110.95 | 98.0 | 2112.0 | [M + 2H] |
| | NEtCONH2G-N-Phe4COOH-Trp7CN-aMeS- | | | 126N2 | | | | + |
| | Phe4CONH2-Phe3CONH2-Sar- | | | 4O22 | | | | |
| | SbMeW7OMe) | | | | | | | |
| 11 | cyclo(bhcLeu-Nle-4Pal-Pipt5OH-W- | E | 2160.73 | C108H | 2158.89 | 100.0 | 1080.6 | [M + 2H] |
| | dProt4Tet-N-Phe4COOH-Trp7Cl-aMeS- | | | 123C1N | | | | 2+ |
| | Phe4CONH2-Phe4COOH-Sar-SbMeW7OMe) | | | 24O23 | | | | |
| 12 | cyclo(bhcLeut45diOH-Nle-4Pal-Pipt5OH- | E | 2132.25 | C107H | 2130.89 | 100.0 | 1066.9 | [M + 2H] |
| | Trp7az-dProt4OH-N-Phe4COOH-Trp7CN- | | | 122N2 | | | | 2+ |
| | aMeS-Phe4CONH2-Phe4COOH-Sar- | | | 2O26 | | | | |
| | SbMeW7OMe) | | | | | | | |
| 13 | cyclo(ClAc-Nle-4Pal-Pip-W-NxG42-N-4Pal- | I | 2085.73 | C104H | 2083.85 | 93.5 | 1043.7 | [M + 2H] |
| | Trp7Cl-aMeS-Phe4COOH-Y-Sar- | | | 122C1N | | | | 2+ |
| | SbMeW7OMe-C)-NH2 | | | 21O22S | | | | |
| 14 | cyclo(bhcLeu-Nle-4Pal-Pipt5OH-W- | E | 2112.30 | C109H | 2110.94 | 100.0 | 2112.1 | [M + 2H] |
| | dProt4OMe-N-Phe4COOH-Trp7CN-aMeS- | | | 126N2 | | | | + |
| | Phe4CONH2-Phe4CONH2-Sar- | | | 2O23 | | | | |
| | SbMeW7OMe) | | | | | | | |
| 15 | cyclo(bhcLeu-Nle-4Pal-Pipt5OH-W- | E | 2121.74 | C108H | 2119.90 | 100.0 | 1061.6 | [M + 2H] |
| | dProt4CH2NH2-N-Phe4COOH-Trp7Cl-aMeS- | | | 126C1N | | | | 2+ |
| | Phe4CONH2-Phe4COOH-Sar-SbMeW7OMe) | | | 21O23 | | | | |
| 16 | cyclo(bhcLeu-Ahp-RbOH4Pal-S-W- | E | 2088.24 | C106H | 2086.90 | 100.0 | 1044.9 | [M + 2H] |
| | dProt4OH-N-Phe4COOH-Trp7CN-aMeS- | | | 122N2 | | | | 2+ |
| | Phe4CONH2-Phe4CONH2-Sar- | | | 2O24 | | | | |
| | SbMeW7OMe) | | | | | | | |
| 17 | cyclo(bhcLeu-Nle-4Pal-Pipt5OH-W- | H | 2108.69 | C107H | 2106.87 | 97.1 | 1054.5 | [M + 2H] |
| | dProt4OH-N-Phe4COOH-Trp7Cl-aMeS- | | | 123C1N | | | | 2+ |
| | Phe4CONH2-Phe3COOH-Sar-SbMeW7OMe) | | | 20O24 | | | | |
| 18 | cyclo(bhcLeu-Nle-4Pal-Pipt5OH- | E | 2252.42 | C111H | 2250.91 | 99.3 | 1126.9 | [M + 2H] |
| | TrpCH2COOH-dProt4NH2-N-TyrOcm- | | | 130N2 | | | | 2+ |
| | Trp7CN-aMeS-F4SO2NH2-TyrOcm-Sar- | | | 2O28S | | | | |
| | SbMeW7OMe) | | | | | | | |
| 19 | cyclo(bhcLeu-Nle-4Pal-Pipt5OH-W-NMeE-N- | H | 2128.30 | C109H | 2126.93 | 100.0 | 1065.1 | [M + 2H] |
| | Phe4COOH-Trp7CN-aMeS-Phe4CONH2- | | | 126N2 | | | | 2+ |
| | Phe4CONH2-Sar-SbMeW7OMe) | | | 2O24 | | | | |
| 20 | cyclo(bhcLeut45diOH-Nle-4Pal-Pip2c-W- | E | 2085.66 | C104H | 2083.87 | 90.0 | 695.8 | [M + 2H] |
| | NMeE-N-4Pal-Trp7Cl-aMeS-Phe4COOH-Y- | | | 122C1N | | | | 3+ |
| | Sar-SbMeW7OMe) | | | 21O24 | | | | |
| 21 | cyclo(bhcLeu-Nle-4Pal-Pipt5OH-W-dProt4F- | E | 2100.26 | C108H | 2098.92 | 92.0 | 2100.0 | [M + 2H] |
| | N-Phe4COOH-Trp7CN-aMeS-Phe4CONH2- | | | 123FN | | | | + |
| | Phe3CONH2-Sar-SbMeW7OMe) | | | 22O22 | | | | |
| 22 | cyclo(bhcLeu-Nle-4Pal-Pipt5OH- | E | 2225.37 | C113H | 2223.95 | 98.0 | 1113.5 | [M + 2H] |
| | TrpCH2COOH-dProt4OAcOH-N- | | | 129N2 | | | | 2+ |
| | Ala4IsoindOne-Trp7CN-aMeS-Phe4CONH2- | | | 3O26 | | | | |
| | Phe4CONH2-Sar-SbMeW7OMe) | | | | | | | |
| 23 | cyclo(bhcLeu-Nle-4Pal-Pipt5OH-Trp7az- | M | 2101.28 | C107H | 2099.93 | 90.0 | 1051.0 | [M + 2H] |
| | N2SPrOHG-N-Phe4COOH-Trp7CN-aMeS- | | | 125N2 | | | | 2+ |
| | Phe4CONH2-Phe4CONH2-Sar- | | | 3O23 | | | | |
| | SbMeW7OMe) | | | | | | | |
| 24 | cyclo(bhcLeu-Nle-4Pal-Pip2c-W-NMeE-N- | H | 2083.69 | C105H | 2081.89 | 100.0 | 695.1 | [M + 2H] |
| | 4Pal-Trp7Cl-aMeS-Phe4COOH-Tyr3OMe- | | | 124C1N | | | | 3+ |
| | Sar-SbMeW7OMe) | | | 21O23 | | | | |
| 25 | cyclo(bhcLeu-Nle-4Pal-Pipt5OH-W- | H | 2072.24 | C106H | 2070.91 | 98.0 | 2071.9 | [M + 2H] |
| | dProt4OH-SbOHAsn-4Pal-Trp7CN-aMeS- | | | 122N2 | | | | + |
| | Phe4CONH2-Phe4COOH-Sar-SbMeW7OMe) | | | 2O23 | | | | |
| 26 | cyclo(bhcLeu-Nle-4Pal-Pipt5OH-Trp7az- | E | 2089.27 | C106H | 2087.93 | 90.7 | 1045.4 | [M + 2H] |
| | dProt4OH-N-BCP3CO2H-Trp7CN-aMeS- | | | 125N2 | | | | 2+ |
| | Phe4CONH2-Phe4CONH2-Sar- | | | 3O23 | | | | |
| | SbMeW7OMe) | | | | | | | |
| 27 | cyclo(bhcLeu-Nle-4Pal-Pipt5OH-W- | H | 2106.72 | C108H | 2104.89 | 95.0 | 1053.4 | [M + 2H] |
| | dProt4OH-N-Phe4COOH-Trp7Cl-aMeS- | | | 125C1N | | | | 2+ |
| | Phe4CONH2-Phe4COOH-Sar-SbPrW) | | | 20O23 | | | | |
| 28 | cyclo(bhcLeu-Nle-4Pal-Pip2c-W-NMeE-N- | E | 2053.66 | C104H | 2051.88 | 100.0 | 1O27.6 | [M + 2H] |
| | 4Pal-Trp7Cl-aMeS-Phe3COOH-Y-Sar- | | | 122C1N | | | | 2+ |
| | SbMeW7OMe) | | | 21O22 | | | | |
| 29 | cyclo(ClAc-Nle-4Pal-NMeT-Trp7az-NMeE-N- | A | 2106.70 | C102H | 2104.83 | 92.2 | 1054.1 | [M + 2H] |
| | 4Pal-Trp7Cl-aMeS-Phe4COOH-Y-Sar- | | | 121C1N | | | | 2+ |
| | SbMeW7OMe-C)-NH2 | | | 22O24S | | | | |
| 30 | cyclo(bhcLeu-Nle-4Pal-Pipt5OH-Trp7az- | F | 2196.35 | C108H | 2194.89 | 99.4 | 1098.7 | [M + 2H] |
| | dProt4OH-N-TyrOcm-Trp7CN-aMeS- | | | 126N2 | | | | 2+ |
| | F4SO2NH2-TyrOcm-Sar-SbMeW7OMe) | | | 2O27S | | | | |
| 31 | cyclo(bhcLeu-Nle-4Pal-Pipt5OH-Trp7az- | E | 2151.31 | C106H | 2149.88 | 98.8 | 1076.4 | [M + 2H] |
| | dProt40AcOH-N-4Pal-Trp7CN-aMeS- | | | 123N2 | | | | 2+ |
| | F4SO2NH2-Phe4COOH-Sar-SbMeW7OMe) | | | 3O25S | | | | |
| 32 | cyclo(bhcLeu-Nle-4Pal-Pipt5OH- | F | 2184.37 | C111H | 2182.97 | 95.0 | 2183.6 | [M + 2H] |
| | Trp7azCH2COOH-dProt4NMe2-N- | | | 130N2 | | | | + |
| | Phe4COOH-Trp7CN-aMeS-Phe4CONH2- | | | 4O24 | | | | |
| | Phe4CONH2-Sar-SbMeW7OMe) | | | | | | | |
| 33 | cyclo(ClAc-Nle-AlaPent-Pip2c-Trp7az-NMeE- | A | 2092.72 | C102H | 2090.85 | 99.2 | 1047.1 | [M + 2H] |
| | N-4Pal-Trp7Cl-aMeS-Phe4COOH-Y-Sar- | | | 123C1N | | | | 2+ |
| | SbMeW7OMe-C)-NH2 | | | 22O23S | | | | |
| 34 | cyclo(bhcLeut4OH-Nle-4Pal-Pip2c-W-NMeE- | E | 2069.66 | C104H | 2067.87 | 93.9 | 1035.7 | [M + 2H] |
| | N-4Pal-Trp7Cl-aMeS-Phe4COOH-Y-Sar- | | | 122C1N | | | | 2+ |
| | SbMeW7OMe) | | | 21O23 | | | | |
| 35 | cyclo(bhcLeu-hR-4Pal-Pip2c-W-NMeE-N- | E | 2110.72 | C105H | 2108.91 | 95.0 | 1056.3 | [M + 2H] |
| | 4Pal-Trp7Cl-aMeS-Phe4COOH-Y-Sar- | | | 125C1N | | | | 2+ |
| | SbMeW7OMe) | | | 24O22 | | | | |
| 36 | cyclo(CIAc-K-Y-S-SbMeW-NMeA-N-4Pal- | D | 1985.18 | C99H1 | 1983.84 | 100.0 | 993.0 | [M + 2H] |
| | W-aMeS-Phe4COOH-Y-Sar-W-C)-NH2 | | | 17N21 | | | | 2+ |
| | | | | O22S | | | | |
| 37 | cyclo(bhcLeu-Nle-PyrimAla-Pipt5OH-W- | F | 2195.37 | C108H | 2193.90 | 97.2 | 1098.4 | [M + 2H] |
| | dProt4NH2-N-TyrOcm-Trp7CN-aMeS- | | | 127N2 | | | | 2+ |
| | F4SO2NH2-TyrOcm-Sar-SbMeW7OMe) | | | 3O26S | | | | |
| 38 | cyclo(CLAc-Nle-4Pal-NEtAla-Trp7az-NMeE- | A | 2090.70 | C102H | 2088.84 | 96.6 | 1045.8 | [M + 2H] |
| | N-4Pal-Trp7Cl-aMeS-Phe4COOH-Y-Sar- | | | 121C1N | | | | 2+ |
| | SbMeW7OMe-C)-NH2 | | | 22O23S | | | | |
| 39 | cyclo(ClAc-Nle-4Pal-Pipt5OH-W-NMeE-N- | A | 2161.19 | C104H | 2158.80 | 100.0 | 1081.1 | [M + 2H] |
| | 4Pal-Trp7Br-aMeS-Phe4CONH2-Y-Sar- | | | 123Br | | | | 2+ |
| | SbMeW7OMe-C)-NH2 | | | N22O2 | | | | |
| | | | | 3S | | | | |
| 40 | cyclo(bhcLeu-Nle-3Pal-Pip2c-W-NMeE-N- | H | 2053.66 | C104H | 2051.88 | 90.9 | 1028.0 | [M + 2H] |
| | 4Pal-Trp7Cl-aMeS-Phe4COOH-Y-Sar- | | | 122C1N | | | | 2+ |
| | SbMeW7OMe) | | | 21O22 | | | | |
| 41 | cyclo(bhcLeu-Nle-4Pal-Pipt5OH-W- | E | 2144.68 | C107H | 2142.85 | 100.0 | 1072.8 | [M + 2H] |
| | dProt4OH-N-Phe2F4COOH-Trp7Cl-aMeS- | | | 121CIF | | | | 2+ |
| | Phe4CONH2-Phe2F4COOH-Sar- | | | 2N20O | | | | |
| | SbMeW7OMe) | | | 24 | | | | |
| 42 | cyclo(ClAc-Nle-4Pal-Pip-Trp7az-NXG27-N- | I | 2088.73 | C103H | 2086.86 | 95.0 | 1045.1 | [M + 2H] |
| | 4Pal-Trp7Cl-aMeS-Phe4COOH-Y-Sar- | | | 123C1N | | | | 2+ |
| | SbMeW7OMe-C)-NH2 | | | 22O22 | | | | |
| | | | | S | | | | |
| 43 | cyclo(bhcLeu-Nle-PyrimAla-Pipt5OH-1Inda- | F | 2239.44 | C110H | 2237.95 | 94.0 | 1120.0 | [M + 2H] |
| | dProt4NMe3-N-TyrOcm-Trp7CN-aMeS- | | | 133N2 | | | | 2+ |
| | F4SO2NH2-TyrOcm-Sar-SbMeW7OMe) | | | 4O26S | | | | |
| 44 | cyclo(bhcLeu-Nle-4Pal-Pipt5OH-W-NMeE-N- | F | 2140.74 | C108H | 2138.90 | 100.0 | 1071.2 | [M + 2H] |
| | TyrOcm-Trp7Cl-aMeS-Phe4CONH2-Y-Sar- | | | 127C1N | | | | 2+ |
| | SbMeW7OMe) | | | 20O25 | | | | |
| 45 | cyclo(bhcLeu-Nle-4Pal-Pipt5OH-W- | E | 2098.27 | C108H | 2096.92 | 98.0 | 1049.7 | [M + 2H] |
| | dProt4OH-N-Phe4COOH-Trp7CN-aMeS- | | | 124N2 | | | | 2+ |
| | Phe4CONH2-Phe4CONH2-Sar- | | | 2O23 | | | | |
| | SbMeW7OMe) | | | | | | | |
| 46 | cyclo(ClAc-Nle-SbMeY-S-Trp7az-NMeE-N- | A | 2107.69 | C102H | 2105.82 | 100.0 | 1054.6 | [M + 2H] |
| | 4Pal-Trp7Cl-aMeS-Phe4COOH-Y-Sar- | | | 120C1N | | | | 2+ |
| | SbMeW7OMe-C)-NH2 | | | 21O25S | | | | |
| 47 | cyclo(bhcLeu-LysMe3-4Pal-Pip-W-NMeE-N- | E | 2110.78 | C108H | 2108.95 | 99.0 | 1055.7 | [M + 2H] |
| | 4Pal-Trp7Cl-aMeS-Phe4COOH-Y-Sar- | | | 131C1N | | | | 2+ |
| | SbMeW7OMe) | | | 21O22 | | | | |
| 48 | cyclo(bhcLeu-Nle-4Pal-Pipt5OH-W- | E | 2093.73 | C107H | 2091.91 | 100.0 | 1047.7 | [M + 2H] |
| | dProt4OH-N-Phe4COOH-Trp7Cl-aMeS- | | | 126C1N | | | | 2+ |
| | Phe4CONH2-Phe4CH2NH2-Sar- | | | 21O22 | | | | |
| | SbMeW7OMe) | | | | | | | |
| 49 | cyclo(bhcLeu-Ahp-SbMe4Pal-MO2-W- | E | 2162.38 | C109H | 2160.92 | 98.0 | 1081.9 | [M + 2H] |
| | dProt4OH-N-Phe4COOH-Trp7CN-aMeS- | | | 128N2 | | | | 2+ |
| | Phe4CONH2-Phe4CONH2-Sar- | | | 2O24S | | | | |
| | SbMeW7OMe) | | | | | | | |
| 50 | cyclo(bhcLeu-Ahp-SbMe4Pal-hS-W- | E | 2100.29 | C108H | 2098.94 | 97.0 | 1050.9 | [M + 2H] |
| | dProt4OH-N-Phe4COOH-Trp7CN-aMeS- | | | 126N2 | | | | 2+ |
| | Phe4CONH2-Phe4CONH2-Sar- | | | 2O23 | | | | |
| | SbMeW7OMe) | | | | | | | |
| 51 | cyclo(bhcLeu-Nle-PyrimAla-Pipt5OH-Trp7az- | E | 2173.27 | C105H | 2171.84 | 100.0 | 1087.2 | [M + 2H] |
| | dProt4OH-N-Phe2F4COOH-Trp7CN-aMeS- | | | 119F2 | | | | 2+ |
| | F4SO2NH2-Phe2F4COOH-Sar- | | | N23O2 | | | | |
| | SbMeW7OMe) | | | 5S | | | | |
| 52 | cyclo(ClAc-Nle-4Pal-Pip2c-TrpCH2COOH- | A | 22O2.79 | C107H | 2200.85 | 100.0 | 1102.4 | [M + 2H] |
| | NMeE-N-Phe4COOH-Trp7Cl-aMeS- | | | 125C1N | | | | 2+ |
| | Phe4CONH2-Y-Sar-SbMeW7OMe-C)-NH2 | | | 22O26S | | | | |
| 53 | cyclo(bhcLeu-Nle-4Pal-Pipt5OH-W- | F | 2065.67 | C105H | 2063.88 | 95.0 | 1033.8 | [M + 2H] |
| | dProt4OH-N-4Pal-Trp7Cl-aMeS- | | | 122C1N | | | | 2+ |
| | Phe4CONH2-Phe4COOH-Sar-SbMeW7OMe) | | | 21O22 | | | | |
| 54 | cyclo(bhcLeu-Cit-4Pal-Pipt5OH-W- | E | 2109.69 | C105H | 2107.88 | 99.0 | 1055.8 | [M + 2H] |
| | dProt4OH-N-4Pal-Trp7Cl-aMeS-Phe4COOH- | | | 122C1N | | | | 2+ |
| | Phe3CONH2-Sar-SbMeW7OMe) | | | 23O23 | | | | |
| 55 | cyclo(bhcLeu-Ahp-SbMe4Pal-D-W- | E | 2114.27 | C108H | 2112.92 | 96.7 | 1057.8 | [M + 2H] |
| | dProt4OH-N-Phe4COOH-Trp7CN-aMeS- | | | 124N2 | | | | 2+ |
| | Phe4CONH2-Phe4CONH2-Sar- | | | 2O24 | | | | |
| | SbMeW7OMe) | | | | | | | |
| 56 | cyclo(ClAc-Nle-4Pal-Cbg-Trp7az-NMeE-N- | A | 2072.69 | C102H | 2070.83 | 86.0 | 1037.0 | [M + 2H] |
| | 4Pal-Trp7Cl-aMeS-Phe4COOH-Y-Sar-aMeW- | | | 119C1N | | | | 2+ |
| | C)-NH2 | | | 22O22S | | | | |
| 57 | cyclo(bhcLeu-Nle-4Pal-Pipt5OH-Trp7az- | F | 2110.67 | C105H | 2108.86 | 98.2 | 1055.9 | [M + 2H] |
| | dProt4OH-N-3Pal4CO2H-Trp7Cl-aMeS- | | | 121C1N | | | | 2+ |
| | Phe4CONH2-Phe4COOH-Sar-SbMeW7OMe) | | | 22O24 | | | | |
| 58 | cyclo(ClAc-Nle-4Pal-Pip-Trp7az-NMeE-N- | A | 2115.80 | C105H | 2113.91 | 98.3 | 1058.4 | [M + 2H] |
| | Phe4CH2NH2-Trp7Cl-aMeS-Phe4CH2NH2- | | | 128C1N | | | | 2+ |
| | Y-Sar-SbMeW7OMe-C)-NH2 | | | 23O21S | | | | |
| 59 | cyclo(ClAc-Nle-Phe4CH2NH2-NMeS-Trp7az- | B | 2118.76 | C104H | 2116.87 | 86.8 | 1060.2 | [M + 2H] |
| | NEtAla-N-Phe4COOH-Trp7Cl-aMeS- | | | 125C1N | | | | 2+ |
| | Phe4CONH2-Y-Sar-SbMeW7OMe-C)-NH2 | | | 22O23S | | | | |
| 60 | cyclo(2SBrBu-Nle-4Pal-Morpip6diMe-W- | A | 2159.81 | C107H | 2157.89 | 95.1 | 1080.8 | [M + 2H] |
| | NMeE-N-4Pal-Trp7Cl-aMeS-Phe4COOH-Y- | | | 128C1N | | | | 2+ |
| | Sar-SbMeW7OMe-C)-NH2 | | | 21O24S | | | | |
| 61 | cyclo(ClAc-Nle-4Pal-Pip-Trp7az-NxG41-N- | I | 2102.76 | C104H | 2100.87 | 98.0 | 1052.1 | [M + 2H] |
| | 4Pal-Trp7Cl-aMeS-Phe4COOH-Y-Sar- | | | 125C1N | | | | 2+ |
| | SbMeW7OMe-C)-NH2 | | | 22O22S | | | | |
| 62 | cyclo(ClAc-Nle-4Pal-Pip-Trp7az-NxG29-N- | I | 2074.70 | C102H | 2072.84 | 98.5 | 1038.2 | [M + 2H] |
| | 4Pal-Trp7Cl-aMeS-Phe4COOH-Y-Sar- | | | 121C1N | | | | 2+ |
| | SbMeW7OMe-C)-NH2 | | | 22O22S | | | | |
| 63 | cyclo(bhcLeu-Nle-4Pal-Pip2c-W-NMeE-N- | G | 2163.82 | C110H | 2161.96 | 85.0 | 1082.4 | [M + 2H] |
| | 4Pal-Trp7Cl-aMeS-Phe4COOH-Phe4AcPip- | | | 132C1N | | | | 2+ |
| | Sar-SbMeW7OMe) | | | 23O22 | | | | |
| 64 | cyclo(bhcLeu-Nle-4Pal-Pip2c-W-NMeE-MO2- | H | 2102.75 | C105H | 2100.86 | 95.0 | 2101.7 | [M + 2H] |
| | 4Pal-Trp7Cl-aMeS-Phe4COOH-Y-Sar- | | | 125C1N | | | | + |
| | SbMeW7OMe) | | | 20O23S | | | | |
| 65 | cyclo(ClAc-Nle-PyrimAla-Pip2c-Trp7az- | A | 2104.69 | C101H | 2102.83 | 100.0 | 1053.2 | [M + 2H] |
| | NMeE-N-4Pal-Trp7Cl-aMeS-Phe4COOH-Y- | | | 119C1N | | | | 2+ |
| | Sar-SbMeW7OMe-C)-NH2 | | | 24O23S | | | | |
| 66 | cyclo(ClAc-Nle-AlapentOH-hS-Trp1Me- | A | 2110.73 | C103H | 2108.85 | 100.0 | 1055.8 | [M + 2H] |
| | NMeE-N-4Pal-Trp7Cl-aMeS-Phe4COOH-Y- | | | 125C1N | | | | 2+ |
| | Sar-SbMeW7OMe-C)-NH2 | | | 20O25S | | | | |
| 67 | cyclo(bhcLeu-Nle-4Pal-Pipc5OH-W- | E | 2064.69 | C105H | 2062.89 | 97.0 | 1033.1 | [M + 2H] |
| | dProt4OH-N-4Pal-Trp7Cl-aMeS- | | | 123C1N | | | | 2+ |
| | Phe4CONH2-Phe3CONH2-Sar- | | | 22O21 | | | | |
| | SbMeW7OMe) | | | | | | | |
| 68 | cyclo(ClAc-Nle-4Pal-Pip2c-Trp7az-NMeE-N- | A | 2103.70 | C102H | 2101.83 | 100.0 | 1052.6 | [M + 2H] |
| | 4Pal-Trp7Cl-aMeS-Phe4COOH-Y-Sar- | | | 120C1N | | | | 2+ |
| | SbMeW7OMe-C)-NH2 | | | 23O23S | | | | |
| 69 | cyclo(cAcBu-Nle-4Pal-Pip2c-W-NMeE-N- | E | 2039.64 | C103H | 2039.86 | 99.1 | 1020.7 | [M + 2H] |
| | 4Pal-Trp7Cl-aMeS-Phe4COOH-Y-Sar- | | | 120C1N | | | | 2+ |
| | SbMeW7OMe) | | | 21O22 | | | | |
| 70 | cyclo(bhcLeu-Ahp-SbMe4Pal-Q-W- | E | 2127.31 | C109H | 2125.95 | 100.0 | 1064.6 | [M + 2H] |
| | dProt4OH-N-Phe4COOH-Trp7CN-aMeS- | | | 127N2 | | | | 2+ |
| | Phe4CONH2-Phe4CONH2-Sar- | | | 3O23 | | | | |
| | SbMeW7OMe) | | | | | | | |
| 71 | cyclo(ClAc-Nle-4Pal-Pipc4OH-W-NMeE-N- | A | 2117.73 | C104H | 2115.84 | 100.0 | 706.5 | [M + 2H] |
| | 4Pal-Trp7Cl-aMeS-Phe4COOH-Y-Sar- | | | 122C1N | | | | 3+ |
| | SbMeW7OMe-C)-NH2 | | | 21O24S | | | | |
| 72 | cyclo(bhcLeu-Nle-4Pal-Pip2c-W-NxG21-N- | E | 2011.63 | C102H | 2009.87 | 86.4 | 1006.7 | [M + 2H] |
| | 4Pal-Trp7Cl-aMeS-Phe4COOH-Y-Sar- | | | 120C1N | | | | 2+ |
| | SbMeW7OMe) | | | 21O21 | | | | |
| 73 | cyclo(1S4RAcpc-hF-4Pal-S-W-NMeA-N- | G | 1938.10 | C102H | 1936.84 | 96.8 | 647.4 | [M + 2H] |
| | 4Pal-W-aMeS-Phe4COOH-Y-Sar-W) | | | 112N2 | | | | 3+ |
| | | | | 0O20 | | | | |
| 74 | cyclo(bhcLeu-Nle-4Pal-Pipt5OH-1Inda- | F | 2068.68 | C104H | 2066.89 | 99.4 | 1035.2 | [M + 2H] |
| | NMeE-N-4Pal-Trp7Cl-aMeS-Phe4CONH2-Y- | | | 123C1N | | | | 2+ |
| | Sar-SbMeW7OMe) | | | 22O22 | | | | |
| 75 | cyclo(ClAc-Nle-4Pal-Pip2c-W-NMeE-Qdm- | A | 2144.79 | C106H | 2142.89 | 90.0 | 715.6 | [M + 2H] |
| | 4Pal-Trp7Cl-aMeS-Phe4COOH-Y-Sar- | | | 127C1N | | | | 3+ |
| | SbMeW7OMe-C)-NH2 | | | 22O23S | | | | |
| 76 | cyclo(ClAc-Nle-4Pal-Pip2c-W-daMeP-N-4Pal- | A | 2070.72 | C103H | 2068.85 | 99.1 | 1035.8 | [M + 2H] |
| | Trp7Cl-aMeS-Phe4COOH-Y-Sar- | | | 121C1N | | | | 2+ |
| | SbMeW7OMe-C)-NH2 | | | 22O21S | | | | |
| 77 | cyclo(bhcLeu-Nle-4Pal-Pipt5OH-Trp7az- | F | 2157.30 | C109H | 2155.92 | 90.0 | 2157.1 | [M + 2H] |
| | dProt40AcOH-N-Phe4COOH-Trp7CN-aMeS- | | | 125N2 | | | | + |
| | Phe4CONH2-Phe4CONH2-Sar- | | | 3O25 | | | | |
| | SbMeW7OMe) | | | | | | | |
| 78 | cyclo(bhcLeu-Nle-4Pal-Pipt5OH-W- | H | 2072.24 | C106H | 2070.91 | 99.6 | 2071.9 | [M + 2H] |
| | dProt4OH-RbOHAsn-4Pal-Trp7CN-aMeS- | | | 122N2 | | | | + |
| | Phe4CONH2-Phe4COOH-Sar-SbMeW7OMe) | | | 2O23 | | | | |
| 79 | cyclo(bhcLeu-Ahp-SbMe4Pal-AbuCN-W- | E | 2109.30 | C109H | 2107.94 | 97.6 | 1055.5 | [M + 2H] |
| | dProt4OH-N-Phe4COOH-Trp7CN-aMeS- | | | 125N2 | | | | 2+ |
| | Phe4CONH2-Phe4CONH2-Sar- | | | 3O22 | | | | |
| | SbMeW7OMe) | | | | | | | |
| 80 | cyclo(bhcLeu-Nle-4Pal-Pip2c-W-NMeE-N- | F | 2083.69 | C105H | 2081.89 | 95.0 | 1042.6 | [M + 2H] |
| | 4Pal-Trp7Cl-aMeS-TyrOcm-Y-Sar- | | | 124C1N | | | | 2+ |
| | SbMeW7OMe) | | | 21O23 | | | | |
| 81 | cyclo(bhcLeu-Anon-4Pal-Pip2c-W-NMeE-N- | F | 2095.74 | C107H | 2093.92 | 90.0 | 1048.9 | [M + 2H] |
| | 4Pal-Trp7Cl-aMeS-Phe4COOH-Y-Sar- | | | 128C1N | | | | 2+ |
| | SbMeW7OMe) | | | 21O22 | | | | |
| 82 | cyclo(bhcLeu-Nle-4Pal-Pip2c-W-NxG29-N- | I | 2O25.65 | C103H | 2O23.88 | 100.0 | 1013.7 | [M + 2H] |
| | 4Pal-Trp7Cl-aMeS-Phe4COOH-Y-Sar- | | | 122C1N | | | | 2+ |
| | SbMeW7OMe) | | | 21O21 | | | | |
| 83 | cyclo(bhcLeu-Nle-4Pal-Pip2c-W-dProt4OH- | H | 2050.66 | C104H | 2048.88 | 98.5 | 684.0 | [M + 2H] |
| | N-4Pal-Trp7Cl-aMeS-Phe4CONH2- | | | 121C1N | | | | 3+ |
| | Phe4COOH-Sar-SbMeW7OMe) | | | 22O21 | | | | |
| 84 | cyclo(bhcLeu-Ahp-4Pal-Pipt5OH-W- | E | 2139.37 | C111H | 2137.98 | 92.8 | 1070.5 | [M + 2H] |
| | dProc4NMe2-N-Phe4COOH-Trp7CN-aMeS- | | | 131N2 | | | | 2+ |
| | Phe4CONH2-Phe3CONH2-Sar- | | | 3O22 | | | | |
| | SbMeW7OMe) | | | | | | | |
| 85 | cyclo(bhcLeu-Ahp-4Pal-Pip2c-W-dProt4OH- | F | 2152.16 | C107H | 2149.84 | 94.4 | 1076.5 | [M + 2H] |
| | N-Phe4COOH-Trp7Br-aMeS-Phe4COOH- | | | 124Br | | | | 2+ |
| | Phe3CONH2-Sar-SbMeW7OMe) | | | N21O23 | | | | |
| 86 | cyclo(bhcLeu-LysMe-4Pal-Pipt5OH-W- | E | 2096.73 | C106H | 2094.92 | 97.0 | 1049.2 | [M + 2H] |
| | NMeE-N-4Pal-Trp7Cl-aMeS-Phe4CONH2-Y- | | | 127C1N | | | | 2+ |
| | Sar-SbMeW7OMe) | | | 22O22 | | | | |
| 87 | cyclo(bhcLeu-Nle-4Pal-Pipt5OH- | F | 2171.32 | C110H | 2169.94 | 100.0 | 1086.1 | [M + 2H] |
| | TrpCH2COOH-dProt4NH2-N-Phe4COOH- | | | 127N2 | | | | 2+ |
| | Trp7CN-aMeS-Phe4CONH2- | | | 3O25 | | | | |
| | RbOHPhe4CONH2-Sar-SbMeW7OMe) | | | | | | | |
| 88 | cyclo(bhcLeu-Nle-4Pal-Pipt5OH-W- | H | 2110.67 | C105H | 2108.86 | 97.4 | 2109.7 | [M + 2H] |
| | dProt4OH-N-AlaPyrim4COOH-Trp7Cl-aMeS- | | | 121C1N | | | | + |
| | Phe4CONH2-Phe4COOH-Sar-SbMeW7OMe) | | | 22O24 | | | | |
| 89 | cyclo(bhcLeu-Nle-PyrimAla-Pipt5OH- | E | 2256.36 | C108H | 2254.88 | 95.2 | 1129.0 | [M + 2H] |
| | 1Inda7az-dProt4OAcOH-N-TyrOcm-Trp7CN- | | | 126N2 | | | | 2+ |
| | aMeS-F4SO2NH2-TyrOcm-Sar- | | | 4O29S | | | | |
| | SbMeW7OMe) | | | | | | | |
| 90 | cyclo(bhcLeu-Nle-4Pal-Pipt5OH-Trp7az- | H | 2131.26 | C107H | 2129.91 | 94.7 | 2130.9 | [M + 2H] |
| | dProt4OH-N-Phe4COOH-Trp7CN-aMeS- | | | 123N2 | | | | + |
| | SbOHPhe4CONH2-RbOHPhe4CONH2-Sar- | | | 3O25 | | | | |
| | SbMeW7OMe) | | | | | | | |
| 91 | cyclo(bhcLeu-Ahp-SbMe4Pal-alT-W- | E | 2100.29 | C108H | 2098.94 | 98.0 | 1051.0 | [M + 2H] |
| | dProt4OH-N-Phe4COOH-Trp7CN-aMeS- | | | 126N2 | | | | 2+ |
| | Phe4CONH2-Phe4CONH2-Sar- | | | 2O23 | | | | |
| | SbMeW7OMe) | | | | | | | |
| 92 | cyclo(bhcLeu-Ahp-SbMe4Pal-N-W- | E | 2113.29 | C108H | 2111.93 | 95.0 | 1057.5 | [M + 2H] |
| | dProt4OH-N-Phe4COOH-Trp7CN-aMeS- | | | 125N2 | | | | 2+ |
| | Phe4CONH2-Phe4CONH2-Sar- | | | 3O23 | | | | |
| | SbMeW7OMe) | | | | | | | |
| 93 | cyclo(ClAc-hhL-4Pal-Pip2c-W-NMeE-N- | A | 2172.80 | C107H | 2170.88 | 100.0 | 1087.4 | [M + 2H] |
| | Phe4COOH-Trp7Cl-aMeS-Phe4CONH2-Y- | | | 127C1N | | | | 2+ |
| | Sar-SbMeW7OMe-C)-NH2 | | | 22O24S | | | | |
| 94 | cyclo(bhcLeu-Nle-4Pal-Pip2c-W-NEtG-N- | F | 1995.63 | C102H | 1993.87 | 98.3 | 998.6 | [M + 2H] |
| | 4Pal-Trp7Cl-aMeS-Phe4COOH-Y-Sar- | | | 120C1N | | | | 2+ |
| | SbMeW7OMe) | | | 21O20 | | | | |
| 95 | cyclo(bhcLeu-Nle-4Pal-Pip2c-W- | E | 2123.71 | C107H | 2121.88 | 99.2 | 1062.7 | [M + 2H] |
| | dProt40AcOH-N-Phe4COOH-Trp7Cl-aMeS- | | | 124C1N | | | | 2+ |
| | Phe4CONH2-Y-Sar-SbMeW7OMe) | | | 21O24 | | | | |
| 96 | cyclo(CLAc-Nle-4Pal-Pipt5OH-W-NMeE-N- | B | 2145.78 | C106H | 2143.87 | 100.0 | 1073.7 | [M + 2H] |
| | Phe4CH2NH2-Trp7Cl-aMeS-Phe4COOH-Y- | | | 126C1N | | | | 2+ |
| | Sar-SbMeW7OMe-C)-NH2 | | | 21O24S | | | | |
| 97 | cyclo(ClAc-Nle-4Pal-Pipt5OH-W-NMeE-N- | A | 2100.74 | C104H | 2098.86 | 98.6 | 1051.0 | [M + 2H] |
| | 4Pal-Trp7Cl-aMeS-Phe4CONH2-Y-Sar- | | | 123C1N | | | | 2+ |
| | SbMeW2Me-C)-NH2 | | | 22O22S | | | | |
| 98 | cyclo(bhcLeu-Ahp-SbMe4Pal-H-W- | E | 2136.33 | C110H | 2134.95 | 100.0 | 1069.2 | [M + 2H] |
| | dProt4OH-N-Phe4COOH-Trp7CN-aMeS- | | | 126N2 | | | | 2+ |
| | Phe4CONH2-Phe4CONH2-Sar- | | | 4O22 | | | | |
| | SbMeW7OMe) | | | | | | | |
| 99 | cyclo(ClAc-Nle-4Pal-NMehSMe-Trp7az- | A | 2120.73 | C103H | 2118.85 | 100.0 | 1060.9 | [M + 2H] |
| | NMeE-N-4Pal-Trp7Cl-aMeS-Phe4COOH-Y- | | | 123C1N | | | | 2+ |
| | Sar-SbMeW7OMe-C)-NH2 | | | 22O24S | | | | |
| 100 | cyclo(bhcLeu-Nle-4Pal-Pip2c-W-NMeE-N- | H | 2083.69 | C105H | 2081.89 | 92.0 | 2083.2 | [M + 2H] |
| | 40Me3Pal-Trp7Cl-aMeS-Phe4COOH-Y-Sar- | | | 124C1N | | | | + |
| | SbMeW7OMe) | | | 21O23 | | | | |
| 101 | cyclo(bhcLeu-Nle-4Pal-Pipt5OH-W-dProt4F- | E | 2067.66 | C105H | 2065.87 | 100.0 | 2067.0 | [M + 2H] |
| | N-4Pal-Trp7Cl-aMeS-Phe4CONH2- | | | 121CIF | | | | + |
| | Phe4COOH-Sar-SbMeW7OMe) | | | N21O21 | | | | |
| 102 | cyclo(bhcLeu-Nle-4Pal-Pipt5OH-W-NMeE-N- | F | 2068.68 | C104H | 2066.89 | 98.0 | 1035.1 | [M + 2H] |
| | PyrimAla-Trp7Cl-aMeS-Phe4CONH2-Y-Sar- | | | 123C1N | | | | 2+ |
| | SbMeW7OMe) | | | 22O22 | | | | |
| 103 | cyclo(bhcLeu-Hag-4Pal-Pipt5OH-W- | F | 2105.69 | C107H | 2103.87 | 95.0 | 1054.0 | [M + 2H] |
| | dProt4OH-N-Phe4COOH-Trp7Cl-aMeS- | | | 122C1N | | | | 2+ |
| | Phe4CONH2-Phe4CONH2-Sar- | | | 21O23 | | | | |
| | SbMeW7OMe) | | | | | | | |
| 104 | cyclo(bhcLeu-Nle-4Pal-Pipt5OH- | F | 2156.31 | C110H | 2154.93 | 100.0 | 1078.6 | [M + 2H] |
| | TrpCH2COOH-dProt4NH2-N-Phe4COOH- | | | 126N2 | | | | 2+ |
| | Trp7CN-aMeS-Phe4CONH2-Phe4COOH-Sar- | | | 2O25 | | | | |
| | SbMeW7OMe) | | | | | | | |
| 105 | cyclo(bhcLeu-Nle-4PyridaAla-Pipt5OH-W- | E | 2109.68 | C106H | 2107.87 | 100.0 | 1055.3 | [M + 2H] |
| | dProt4OH-N-Phe4COOH-Trp7Cl-aMeS- | | | 122C1N | | | | 2+ |
| | Phe4CONH2-Phe4COOH-Sar-SbMeW7OMe) | | | 21O24 | | | | |
| 106 | cyclo(bhcLeu-Nle-4Pal-Pipt5OH-W- | H | 2122.72 | C108H | 2120.89 | 95.0 | 1061.4 | [M + 2H] |
| | dProt4OH-N-Phe4COOH-Trp7Cl-aMeS- | | | 125C1N | | | | 2+ |
| | Phe4CONH2-Phe4COOH-Sar- | | | 20O24 | | | | |
| | SbMeW2Me7OMe) | | | | | | | |
| 107 | cyclo(bhcLeu-Nle-4Pal-Pip2c-W-NMeE-N- | H | 2099.66 | C105H | 2097.86 | 97.5 | 2098.14 | [M + 2H] |
| | 4Pal-Trp7Cl-aMeS-Phe4COOH- | | | 121CIF | | | | + |
| | Phe2F4COOH-Sar-SbMeW7OMe) | | | N21O23 | | | | |
| 108 | cyclo(bhcLeu-Nle-4Pal-Pip2c-W-NMeE-N- | H | 2114.67 | C106H | 2112.86 | 97.0 | 2114.1 | [M + 2H] |
| | Phe2F4COOH-Trp7Cl-aMeS-Phe4COOH-Y- | | | 122CIF | | | | + |
| | Sar-SbMeW7OMe) | | | N20O24 | | | | |
| 109 | cyclo(bhcLeu-Ahp-4Pal-Pip2c-W-dProt4OH- | F | 2151.18 | C107H | 2148.85 | 95.3 | 1076.1 | [M + 2H] |
| | N-Phe4COOH-Trp7Br-aMeS-Phe4CONH2- | | | 125Br | | | | 2+ |
| | Phe3CONH2-Sar-SbMeW7OMe) | | | N22O22 | | | | |
| 110 | cyclo(bhcLeu-Ahp-SbMe4Pal-Cit-W- | E | 2156.36 | C110H | 2154.97 | 98.2 | 1079.0 | [M + 2H] |
| | dProt4OH-N-Phe4COOH-Trp7CN-aMeS- | | | 130N2 | | | | 2+ |
| | Phe4CONH2-Phe4CONH2-Sar- | | | 4O23 | | | | |
| | SbMeW7OMe) | | | | | | | |
| 111 | cyclo(bhcLeu-Nle-4Pal-Pip2c-W-NMeE-N- | E | 2067.69 | C105H | 2065.89 | 100.0 | 1034.7 | [M + 2H] |
| | 4Pal-Trp7Cl-aMeS-F4MCO2H-Y-Sar- | | | 124C1N | | | | 2+ |
| | SbMeW7OMe) | | | 21O22 | | | | |
| 112 | cyclo(bhcLeu-Ahp-4Pal-Pip2c-W-dProt4OH- | H | 2082.68 | C105H | 2080.88 | 90.2 | 1042.2 | [M + 2H] |
| | N-4Pal-Trp6F7Cl-aMeS-Phe4COOH- | | | 122CIF | | | | 2+ |
| | Phe4CONH2-Sar-SbMeW7OMe) | | | N22O21 | | | | |
| 113 | cyclo(bhcLeu-Nle-4Pal-Pipt5OH-Trp7az- | F | 2217.39 | C112H | 2215.98 | 100.0 | 1108.6 | [M + 2H] |
| | dProt4NHC2acidNMe3-N-Phe4COOH- | | | 132FN | | | | 2+ |
| | Trp7CN-aMeS-Phe4CONH2-Phe3F4COOH- | | | 24O24 | | | | |
| | Sar-SbMeW7OMe) | | | | | | | |
| 114 | cyclo(bhcLeu-Nle-4Pal-Pipt5OH-W-dProt4F- | E | 2100.26 | C108H | 2098.92 | 100.0 | 2100.0 | [M + 2H] |
| | N-Phe4COOH-Trp7CN-aMeS-Phe4CONH2- | | | 123FN | | | | + |
| | Phe4CONH2-Sar-SbMeW7OMe) | | | 22O22 | | | | |
| 115 | cyclo(bhcLeu-Nle-4Pal-Pipt5OH- | F | 2239.38 | C109H | 2237.89 | 100.0 | 1120.2 | [M + 2H] |
| | Trp7azCH2COOH-dProt4OAcOH-N-4Pal- | | | 127N2 | | | | 2+ |
| | Trp7CN-aMeS-F4SO2NH2-TyrOcm-Sar- | | | 3O28S | | | | |
| | SbMeW7OMe) | | | | | | | |
| 116 | cyclo(bhcLeu-Nle-4Pal-Pipt5OH-Trp7az- | F | 2217.39 | C112H | 2215.98 | 100.0 | 1108.7 | [M + 2H] |
| | dProt4NHC2acidNMe3-N-Phe3F4COOH- | | | 132FN | | | | 2+ |
| | Trp7CN-aMeS-Phe4CONH2-Phe4COOH-Sar- | | | 24O24 | | | | |
| | SbMeW7OMe) | | | | | | | |
| 117 | cyclo(bhcLeu-Nle-4Pal-Pip2c-W-NMeE-N- | E | 2110.71 | C107H | 2108.89 | 99.8 | 1056.2 | [M + 2H] |
| | Phe4COOMe-Trp7Cl-aMeS-Phe4COOH-Y- | | | 125C1N | | | | 2+ |
| | Sar-SbMeW7OMe) | | | 20O24 | | | | |
| 118 | cyclo(bhcLeu-Nle-SbMePhe4COOH-Cit- | E | 2186.34 | C110H | 2184.95 | 97.6 | 1093.9 | [M + 2H] |
| | Trp7az-dProt4OH-N-Phe4COOH-Trp7CN- | | | 128N2 | | | | 2+ |
| | aMeS-Phe4CONH2-Phe4CONH2-Sar- | | | 4O25 | | | | |
| | SbMeW7OMe) | | | | | | | |
| 119 | cyclo(bhcLeu-Nle-4Pal-Pipt5OH- | H | 2208.36 | C109H | 2206.89 | 96.0 | 2207.9 | [M + 2H] |
| | TrpCH2COOH-dProt4OH-N-F4SO2NH2- | | | 126N2 | | | | + |
| | Trp7CN-aMeS-RbOHPhe4CONH2- | | | 2O27S | | | | |
| | Phe4COOH-Sar-SbMeW7OMe) | | | | | | | |
| 120 | cyclo(bhcLeu-Nle-4Pal-Pipc5CO2H-W- | E | 2096.68 | C106H | 2094.87 | 94.9 | 1049.1 | [M + 2H] |
| | NMeE-N-4Pal-Trp7Cl-aMeS-Phe4COOH-Y- | | | 123C1N | | | | 2+ |
| | Sar-SbMeW7OMe) | | | 20O24 | | | | |
| 121 | cyclo(bhcLeu-Nle-4Pal-Pipt5OH-Trp7az- | H | 2097.25 | C106H | 2095.91 | 97.0 | 2096.9 | [M + 2H] |
| | NEt3OxadG-N-4Pal-Trp7CN-aMeS- | | | 121N2 | | | | + |
| | Phe4CONH2-Phe4COOH-Sar-SbMeW7OMe) | | | 5022 | | | | |
| 122 | cyclo(bhcLeu-Nle-4Pal-Pipt5OH-Trp7az- | H | 2086.26 | C107H | 2084.92 | 97.0 | 2085.9 | [M + 2H] |
| | dProt4OH-alT-Phe4COOH-Trp7CN-aMeS- | | | 124N2 | | | | + |
| | Phe4CONH2-Phe4CONH2-Sar- | | | 2O23 | | | | |
| | SbMeW7OMe) | | | | | | | |
| 123 | cyclo(ClAc-Nle-Phe4CONH2-NMeS-W- | D | 2176.75 | C106H | 2174.83 | 93.0 | 1088.2 | [M + 2H] |
| | NMeE-N-Phe4COOH-Trp7Cl-aMeS- | | | 123C1N | | | | 2+ |
| | Phe4COOH-Y-Sar-SbMeW7OMe-C)-NH2 | | | 20O27S | | | | |
| 124 | cyclo(ClAc-Nle-4Pal-Pip2c-W-NMeE-N-4Pal- | D | 2129.74 | C104H | 2127.85 | 91.3 | 1059.3 | [M + 2H] |
| | Trp7Cl-aMeS-Phe4COOH-Phe4CONH2-Sar- | | | 122C1N | | | | 2+ |
| | SbMeW7OMe-C)-NH2 | | | 23O23S | | | | |
| 125 | cyclo(ClAc-Nle-4Pal-Prot4NH2-Trp7az- | B | 2103.70 | C102H | 2101.83 | 89.1 | 1052.5 | [M + 2H] |
| | NMeE-N-4Pal-Trp7Cl-aMeS-Phe4COOH-Y- | | | 120C1N | | | | 2+ |
| | Sar-SbMeW7OMe-C)-NH2 | | | 23O23S | | | | |
| 126 | cyclo(bhcLeu-Nle-4Pal-Pipt5OH-W-NMeE-N- | F | 2125.72 | C107H | 2123.90 | 100.0 | 1063.2 | [M + 2H] |
| | 4Pal-Trp7Cl-aMeS-Phe4CONH2-TyrOcm- | | | 126C1N | | | | 2+ |
| | Sar-SbMeW7OMe) | | | 21O24 | | | | |
| 127 | cyclo(bhcLeu-Nle-4Pal-Pip2c-W-NMeE-N- | F | 2067.69 | C104H | 2065.90 | 95.0 | 1034.9 | [M + 2H] |
| | 4Pal-Trp7Cl-aMeS-F4urea-Y-Sar- | | | 124C1N | | | | 2+ |
| | SbMeW7OMe) | | | 23O21 | | | | |
| 128 | cyclo(bhcLeu-Ahp-4Pal-Pipt5OH-W- | E | 2139.37 | C111H | 2137.98 | 82.2 | 1070.5 | [M + 2H] |
| | dProt4NMe2-N-Phe4COOH-Trp7CN-aMeS- | | | 131N2 | | | | 2+ |
| | Phe4CONH2-Phe3CONH2-Sar- | | | 3O22 | | | | |
| | SbMeW7OMe) | | | | | | | |
| 129 | cyclo(bhcLeu-Nle-4Pal-Pipt5OH- | E | 2187.32 | C110H | 2185.93 | 100.0 | 1094.4 | [M + 2H] |
| | TrpCH2COOH-dProt4OAcOH-N-RbOH4Pal- | | | 127N2 | | | | 2+ |
| | Trp7CN-aMeS-Phe4CONH2-Phe4CONH2- | | | 3O26 | | | | |
| | Sar-SbMeW7OMe) | | | | | | | |
| 130 | cyclo(bhcLeu-Nle-4Pal-Pipt5OH-W- | H | 2114.27 | C108H | 2112.92 | 90.7 | 1057.7 | [M + 2H] |
| | NEtCONH2G-N-Phe4COOH-Trp7CN-aMeS- | | | 124N2 | | | | 2+ |
| | Phe4CONH2-Phe4COOH-Sar-SbMeW7OMe) | | | 2O24 | | | | |
| 131 | cyclo(bhcLeu-hK-4Pal-Pipt5OH-W-NMeE-N- | H | 2097.71 | C106H | 2095.90 | 98.7 | 1049.3 | [M + 2H] |
| | 4Pal-Trp7Cl-aMeS-Phe4COOH-Y-Sar- | | | 126C1N | | | | 2+ |
| | SbMeW7OMe) | | | 21O23 | | | | |
| 132 | cyclo(bhcLeu-Nle-4Pal-Pipt5OH-W- | E | 2150.73 | C109H | 2148.88 | 100.0 | 1075.7 | [M + 2H] |
| | dProc4CH2COOH-N-Phe4COOH-Trp7Cl- | | | 125C1N | | | | 2+ |
| | aMeS-Phe4CONH2-Phe4COOH-Sar- | | | 20O25 | | | | |
| | SbMeW7OMe) | | | | | | | |
| 133 | cyclo(bhcLeu-Nle-4Pal-Pipt5OH-Trp7az- | H | 2165.34 | C107H | 2163.89 | 90.0 | 2164.9 | [M + 2H] |
| | dNMeE-N-F4SO2NH2-Trp7CN-aMeS- | | | 125N2 | | | | + |
| | Phe4CONH2-Phe4COOH-Sar-SbMeW7OMe) | | | 3O25S | | | | |
| 134 | cyclo(bhcLeu-Nle-4Pal-Pipt5OH-Trp7az- | E | 2230.32 | C108H | 2228.85 | 100.0 | 1115.9 | [M + 2H] |
| | dProt40AcOH-N-Phe2F4COOH-Trp7CN- | | | 122F2 | | | | 2+ |
| | aMeS-F4SO2NH2-Phe2F4COOH-Sar- | | | N22O2 | | | | |
| | SbMeW7OMe) | | | 7S | | | | |
| 135 | cyclo(bhcLeu-Nle-4Pal-Pipt5OH-W- | E | 2195.36 | C109H | 2193.89 | 96.9 | 1098.6 | [M + 2H] |
| | dProt4OH-N-TyrOcm-Trp7CN-aMeS- | | | 127N2 | | | | 2+ |
| | F4SO2NH2-TyrOcm-Sar-SbMeW7OMe) | | | 1O27S | | | | |
| 136 | cyclo(bhcLeu-Nle-4Pal-Pipt5OH- | F | 2278.52 | C114H | 2276.99 | 98.9 | 1139.2 | [M + 2H] |
| | TrpCH2COOH-dProt4OEtNMe3-N- | | | 138N2 | | | | 2+ |
| | Phe4COOH-Trp7CN-aMeS-F4SO2NH2- | | | 3O26S | | | | |
| | Phe4CONH2-Sar-SbMeW7OMe) | | | | | | | |
| 137 | cyclo(bhcLeu-hL-4Pal-Pip2c-W-NMeE-N- | F | 2067.69 | C105H | 2065.89 | 95.0 | 1034.7 | [M + 2H] |
| | 4Pal-Trp7Cl-aMeS-Phe4COOH-Y-Sar- | | | 124C1N | | | | 2+ |
| | SbMeW7OMe) | | | 21O22 | | | | |
| 138 | cyclo(bhcLeu-Nle-4Pal-Pip2c-W-NMeE-N- | F | 2059.69 | C102H | 2057.83 | 95.0 | 1030.6 | [M + 2H] |
| | TZAla-Trp7Cl-aMeS-Phe4COOH-Y-Sar- | | | 120C1N | | | | 2+ |
| | SbMeW7OMe) | | | 21O22S | | | | |
| 139 | cyclo(bhcLeu-Nle-4Pal-Pip2c-TrpCH2COOH- | F | 2241.44 | C114H | 2240.00 | 92.0 | 1121.0 | [M + 2H] |
| | dProt4NHC2acidNMe3-N-Phe4COOH- | | | 135N2 | | | | 2+ |
| | Trp7CN-aMeS-Phe4CONH2-Phe4COOH-Sar- | | | 4O25 | | | | |
| | SbMeW7OMe) | | | | | | | |
| 140 | cyclo(bhcLeu-Ahp-4Pal-Pip2c-W- | E | 2155.33 | C110H | 2153.94 | 100.0 | 1078.25 | [M + 2H] |
| | dProt40AcOH-N-Phe4COOH-Trp7CN-aMeS- | | | 127N2 | | | | 2+ |
| | Phe4CONH2-Phe4CONH2-Sar- | | | 3O24 | | | | |
| | SbMeW7OMe) | | | | | | | |
| 141 | cyclo(bhcLeu-Nle-4Pal-Pip2c-W-Sar-N-4Pal- | F | 1981.60 | C101H | 1979.86 | 100.0 | 991.8 | [M + 2H] |
| | Trp7Cl-aMeS-Phe4COOH-Y-Sar- | | | 118C1N | | | | 2+ |
| | SbMeW7OMe) | | | 21O20 | | | | |
| 142 | cyclo(bhcLeu-SerOEt-4Pal-Pipt5OH-W- | H | 2067.65 | C104H | 2065.86 | 98.0 | 689.3 | [M + 2H] |
| | dProt4OH-N-4Pal-Trp7Cl-aMeS- | | | 120C1N | | | | 3+ |
| | Phe4CONH2-Phe4COOH-Sar-SbMeW7OMe) | | | 21O23 | | | | |
| 143 | cyclo(CLAc-Nle-RbMe4Pal-S-Trp7az-NMeE- | A | 2092.68 | C101H | 2090.82 | 100.0 | 1047.2 | [M + 2H] |
| | N-4Pal-Trp7Cl-aMeS-Phe4COOH-Y-Sar- | | | 119C1N | | | | 2+ |
| | SbMeW7OMe-C)-NH2 | | | 22O24S | | | | |
| 144 | cyclo(bhcLeu-Nle-4Pal-Pipt5OH-W- | F | 2036.68 | C104H | 2034.90 | 98.0 | 1019.2 | [M + 2H] |
| | dProt4NH2-N-4Pal-Trp7Cl-aMeS- | | | 123C1N | | | | 2+ |
| | Phe4CONH2-Y-Sar-SbMeW7OMe) | | | 22O20 | | | | |
| 145 | cyclo(bhcLeu-Nle-4Pal-Pipt5OH-W-NMeE-N- | F | 2125.77 | C108H | 2123.93 | 99.2 | 1063.9 | [M + 2H] |
| | AEF-Trp7Cl-aMeS-Phe4CONH2-Y-Sar- | | | 130C1N | | | | 2+ |
| | SbMeW7OMe) | | | 21O23 | | | | |
| 146 | cyclo(ClAc-OrnN3-Y-Da6-020p-SbMeW-dP-N- | D | 2030.62 | C97H1 | 2028.80 | 80.2 | 1015.4 | [M + 2H] |
| | 4Pal-Trp7Cl-aMeS-Phe4COOH-H-Sar-aMeW- | | | 13C1N2 | | | | 2+ |
| | C)-NH2 | | | 6O20S | | | | |
| 147 | cyclo(bhcLeu-Nle-4Pal-Pipt5OH-Trp7az- | H | 2128.30 | C108H | 2126.94 | 95.0 | 1064.5 | [M + 2H] |
| | NEtCONH2A-N-Phe4COOH-Trp7CN-aMeS- | | | 126N2 | | | | 2+ |
| | Phe4CONH2-Phe4CONH2-Sar- | | | 4023 | | | | |
| | SbMeW7OMe) | | | | | | | |
| 148 | cyclo(bhcLeu-SerOC2NH2-4Pal-Pipt5OH- | F | 2126.67 | C105H | 2124.86 | 98.9 | 1064.1 | [M + 2H] |
| | Trp7az-dProt4OH-N-Phe4COOH-Trp7Cl- | | | 121C1N | | | | 2+ |
| | aMeS-Phe4CONH2-Phe4COOH-Sar- | | | 22O25 | | | | |
| | SbMeW7OMe) | | | | | | | |
| 149 | cyclo(bhcLeu-SerOCH2cPr-4Pal-Pipt5OH- | F | 2128.26 | C108H | 2126.90 | 100.0 | 1064.9 | [M + 2H] |
| | Trp7az-dProt4OH-N-Phe4COOH-Trp7CN- | | | 122N2 | | | | 2+ |
| | aMeS-Phe4CONH2-Phe4COOH-Sar- | | | 2O25 | | | | |
| | SbMeW7OMe) | | | | | | | |
| 150 | cyclo(bhcLeu-Ahp-4Pal-Pipt5OH-W- | E | 2165.36 | C112H | 2163.96 | 99.0 | 1083.5 | [M + 2H] |
| | dProt4NH2-N-Phe4COOH-Trp7CN-aMeS- | | | 129N2 | | | | 2+ |
| | Phe4CONH2-TrpCH2COOH-Sar- | | | 3O23 | | | | |
| | SbMeW7OMe) | | | | | | | |
| 151 | cyclo(CLAc-Nle-4Pal-Pipt5OH-Trp1Me- | A | 2130.77 | C105H | 2128.87 | 100.0 | 1066.2 | [M + 2H] |
| | NMeE-N-4Pal-Trp7Cl-aMeS-Phe4CONH2-Y- | | | 125C1N | | | | 2+ |
| | Sar-SbMeW7OMe-C)-NH2 | | | 22O23S | | | | |
| 152 | cyclo(ClAc-Nle-AlapentOH-T-1 Inda-NMeE- | A | 2097.69 | C101H | 2095.83 | 95.6 | 1049.7 | [M + 2H] |
| | N-4Pal-Trp7Cl-aMeS-Phe4COOH-Y-Sar- | | | 122C1N | | | | 2+ |
| | SbMeW7OMe-C)-NH2 | | | 21O25S | | | | |
| 153 | cyclo(bhcLeu-Nle-4Pal-Pip2c-W-NMeE-N- | E | 2094.71 | C106H | 2092.90 | 98.4 | 1047.9 | [M + 2H] |
| | 4Pal-Trp7Cl-aMeS-Phe4COOH-F3MCONH2- | | | 125C1N | | | | 2+ |
| | Sar-SbMeW7OMe) | | | 22O22 | | | | |
| 154 | cyclo(bhcLeu-Ahp-SbMe4Pal-hSOMe-W- | E | 2114.32 | C109H | 2112.95 | 100.0 | 1058.0 | [M + 2H] |
| | dProt4OH-N-Phe4COOH-Trp7CN-aMeS- | | | 128N2 | | | | 2+ |
| | Phe4CONH2-Phe4CONH2-Sar- | | | 2O23 | | | | |
| | SbMeW7OMe) | | | | | | | |
| 155 | cyclo(bhcLeu-Nle-4Pal-Pipt5OH-W- | H | 2088.24 | C106H | 2086.90 | 97.0 | 2087.9 | [M + 2H] |
| | dProt4OH-N-4Pal-Trp7CN-aMeS- | | | 122N2 | | | | + |
| | SbOHPhe4CONH2-RbOHPhe4COOH-Sar- | | | 2O24 | | | | |
| | SbMeW7OMe) | | | | | | | |
| 156 | cyclo(bhcLeu-Nle-4Pal-Pipt5OH- | E | 2226.36 | C112H | 2224.94 | 98.0 | 1113.9 | [M + 2H] |
| | Trp7azCH2COOH-dProt4OAcOH-N- | | | 128N2 | | | | 2+ |
| | Ala4IsoindOne-Trp7CN-aMeS-Phe4CONH2- | | | 4O26 | | | | |
| | Phe4CONH2-Sar-SbMeW7OMe) | | | | | | | |
| 157 | cyclo(ClAc-Nle-4Pal-Pip-Trp7az-NhEG-N- | A | 2102.71 | C103H | 2100.84 | 98.2 | 1052.1 | [M + 2H] |
| | 4Pal-Trp7Cl-aMeS-Phe4COOH-Y-Sar- | | | 121C1N | | | | 2+ |
| | SbMeW7OMe-C)-NH2 | | | 22O23S | | | | |
| 158 | cyclo(ClAc-Nle-4Pal-Pip2c-Trp7az-NMeE-N- | B | 2171.70 | C103H | 2169.82 | 98.2 | 1086.4 | [M + 2H] |
| | 4Pal-Trp7Cl-aMeS-Phe4COOH-TyrOCF3- | | | 119CIF | | | | 2+ |
| | Sar-SbMeW7OMe-C)-NH2 | | | 3N23O | | | | |
| | | | | 23S | | | | |
| 159 | cyclo(bhcLeu-Nle-4Pal-Pipt5OH-Trp7az- | F | 2067.65 | C103H | 2065.87 | 100.0 | 1034.6 | [M + 2H] |
| | dProt4OH-N-4PyrimAla-Trp7Cl-aMeS- | | | 120C1N | | | | 2+ |
| | Phe4CONH2-Phe4COOH-Sar-SbMeW7OMe) | | | 23O22 | | | | |
| 160 | cyclo(bhcLeu-Nle-4Pal-Pip2c-W-NMeE-N- | G | 2096.73 | C106H | 2094.92 | 97.0 | 1048.8 | [M + 2H] |
| | 4Pal-Trp7Cl-aMeS-Phe4COOH-AEF-Sar- | | | 127C1N | | | | 2+ |
| | SbMeW7OMe) | | | 22O22 | | | | |
| 161 | cyclo(bhcLeu-Nle-4Pal-Pip2c-W-dProt4NH2- | E | 2022.65 | C103H | 2020.88 | 99.1 | 1011.8 | [M + 2H] |
| | N-4Pal-Trp7Cl-aMeS-Phe4COOH-Y-Sar- | | | 121C1N | | | | 2+ |
| | SbMeW7OMe) | | | 22O20 | | | | |
| 162 | cyclo(ClAc-Nle-4Pal-Pip2c-W-NMeE-N- | C | 2158.78 | C106H | 2156.86 | 86.0 | 1080.0 | [M + 2H] |
| | Phe4CONH2-Trp7Cl-aMeS-Phe4COOH- | | | 125C1N | | | | 2+ |
| | TyrOMe-Sar-SbMeW7OMe-C)-NH2 | | | 22O24S | | | | |
| 163 | cyclo(bhcLeu-Nle-4Pal-Pipt5OH-W-NMeE-N- | F | 2067.69 | C105H | 2065.89 | .94.0 | 1034.3 | [M + 2H] |
| | 4Pal-Trp7Cl-aMeS-Phe4CONH2-Y-Sar- | | | 124C1N | | | | 2+ |
| | SbMeW7OMe) | | | 21O22 | | | | |
| 164 | cyclo(CIAc-Nle-Phe4CONH2-Bhv-Trp7az- | A | 2148.74 | C104H | 2146.84 | 98.8 | 1075.4 | [M + 2H] |
| | NMeE-N-4Pal-Trp7Cl-aMeS-Phe4COOH-Y- | | | 123C1N | | | | 2+ |
| | Sar-SbMeW7OMe-C)-NH2 | | | 22O25S | | | | |
| 165 | cyclo(bhcLeu-Nle-4Pal-Pipt5OH-W-NMeE-N- | F | 2124.74 | C107H | 2122.91 | 100.0 | 1063.0 | [M + 2H] |
| | F4urea-Trp7Cl-aMeS-Phe4CONH2-Y-Sar- | | | 127C1N | | | | 2+ |
| | SbMeW7OMe) | | | 22O23 | | | | |
| 166 | cyclo(bhcLeu-FX1-4Pal-NMeS-W-NMeE-N- | J | 2134.69 | C107H | 2132.86 | 96.0 | 1068.2 | [M + 2H] |
| | 4Pal-Trp7Cl-aMeS-Phe4COOH-Y-Sar- | | | 121C1N | | | | 2+ |
| | SbMeW7OMe) | | | 22O24 | | | | |
| 167 | cyclo(ClAc-Nle-4Pal-Pip2c-W-dPro4cPr-N- | A | 2082.73 | C104H | 2080.85 | 100.0 | 1041.7 | [M + 2H] |
| | 4Pal-Trp7Cl-aMeS-Phe4COOH-Y-Sar- | | | 121C1N | | | | 2+ |
| | SbMeW7OMe-C)-NH2 | | | 22O21S | | | | |
| 168 | cyclo(2SBrBu-Nle-4Pal-Pipt5OH-Trp7az- | A | 2188.80 | C107H | 2186.88 | 93.3 | 1095.0 | [M + 2H] |
| | NMeE-N-Phe4COOH-Trp7Cl-aMeS- | | | 127C1N | | | | 2+ |
| | Phe4CONH2-Y-Sar-SbMeW7OMe-C)-NH2 | | | 22O25S | | | | |
| 169 | cyclo(bhcLeu-FX2-4Pal-Pipt5OH-W- | K | 2146.71 | C107H | 2144.87 | 95.0 | 1074.7 | [M + 2H] |
| | dProt4OH-N-4Pal-Trp7Cl-aMeS- | | | 121C1N | | | | 2+ |
| | Phe4CONH2-Phe3CONH2-Sar- | | | 24O23 | | | | |
| | SbMeW7OMe) | | | | | | | |
| 170 | cyclo(CLAc-Nle-4Pal-Pip-Trp7az-NxG38-N- | I | 2074.70 | C102H | 2072.84 | 94.0 | 1038.0 | [M + 2H] |
| | 4Pal-Trp7Cl-aMeS-Phe4COOH-Y-Sar- | | | 121C1N | | | | 2+ |
| | SbMeW7OMe-C)-NH2 | | | 22O22S | | | | |
| 171 | cyclo(bhcLeu-Nle-4Pal-Pip2c-W-NMeE-N- | H | 2146.76 | C106H | 2144.85 | 97.0 | 2146.1 | [M + 2H] |
| | F4mSO3H-Trp7Cl-aMeS-Phe4COOH-Y-Sar- | | | 125C1N | | | | + |
| | SbMeW7OMe) | | | 20O25S | | | | |
| 172 | cyclo(ClAc-Nle-hF-Pip2c-Trp7az-NMeE-N- | A | 2116.74 | C104H | 2114.85 | 94.0 | 1059.1 | [M + 2H] |
| | 4Pal-Trp7Cl-aMeS-Phe4COOH-Y-Sar- | | | 123C1N | | | | 2+ |
| | SbMeW7OMe-C)-NH2 | | | 22O23S | | | | |
| 173 | cyclo(bhcLeu-Nle-4Pal-Pip2c-W-NMeE-N- | H | 2132.74 | C105H | 2130.84 | 98.0 | 2132.0 | [M + 2H] |
| | F4SO3H-Trp7Cl-aMeS-Phe4COOH-Y-Sar- | | | 123C1N | | | | + |
| | SbMeW7OMe) | | | 20O25S | | | | |
| 174 | cyclo(bhcLeu-Ahp-4Pal-Pip2c-W-dProt4OH- | E | 2054.27 | C106H | 2052.94 | 99.6 | 1028.1 | [M + 2H] |
| | N-4Pal-Trp7CN-aMeS-Phe4CONH2- | | | 124N2 | | | | 2+ |
| | Phe3CONH2-Sar-SbMeW7OMe) | | | 4O20 | | | | |
| 175 | cyclo(bhcLeu-Nle-4Pal-Pip2c-W-NMeE-N- | H | 2071.65 | C104H | 2069.87 | 93.0 | 2071.2 | [M + 2H] |
| | 4Pal-Trp7Cl-aMeS-Phe4COOH-Tyr2F-Sar- | | | 121CIF | | | | + |
| | SbMeW7OMe) | | | N21O22 | | | | |
| 176 | cyclo(bhcLeu-Ahp-SbMe4Pal-Apm-W- | E | 2156.35 | C111H | 2154.96 | 99.0 | 1079.0 | [M + 2H] |
| | dProt4OH-N-Phe4COOH-Trp7CN-aMeS- | | | 130N2 | | | | 2+ |
| | Phe4CONH2-Phe4CONH2-Sar- | | | 2O24 | | | | |
| | SbMeW7OMe) | | | | | | | |
| 177 | cyclo(ClAc-Nle-PyrimAla-Bhv-Trp7az- | A | 2107.69 | C101H | 2105.83 | 100.0 | 1054.7 | [M + 2H] |
| | NMeE-N-4Pal-Trp7Cl-aMeS-Phe4COOH-Y- | | | 120C1N | | | | 2+ |
| | Sar-SbMeW7OMe-C)-NH2 | | | 23O24S | | | | |
| 178 | cyclo(bhcLeu-Nle-4Pal-Pip2c-W-NMeE-N- | H | 2O27.63 | C101H | 2O25.87 | 95.0 | 1014.8 | [M + 2H] |
| | 4Pal-Trp7Cl-aMeS-Phe4COOH-H-Sar- | | | 120C1N | | | | 2+ |
| | SbMeW7OMe) | | | 23O21 | | | | |
| 179 | cyclo(CLAc-Nle-4Pal-Morpip6diMe-W-NMeE- | B | 2204.80 | C108H | 22O2.86 | 93.0 | 1103.2 | [M + 2H] |
| | N-TyrOcm-Trp7Cl-aMeS-Phe4COOH-Y-Sar- | | | 127C1N | | | | 2+ |
| | SbMeW7OMe-C)-NH2 | | | 20O27S | | | | |
| 180 | cyclo(ClAc-Nle-SbMe4Pal-S-Trp7az-NMeE- | A | 2092.68 | C101H | 2090.82 | 100.0 | 1047.1 | [M + 2H] |
| | N-4Pal-Trp7Cl-aMeS-Phe4COOH-Y-Sar- | | | 119C1N | | | | 2+ |
| | SbMeW7OMe-C)-NH2 | | | 22O24S | | | | |
| 181 | cyclo(bhcLeu-Nle-4Pal-Pip2c-W-NMeE- | F | 2054.69 | C105H | 2052.90 | 96.5 | 1O27.5 | [M + 2H] |
| | hSOMe-4Pal-Trp7Cl-aMeS-Phe4COOH-Y- | | | 125C1N | | | | 2+ |
| | Sar-SbMeW7OMe) | | | 20O22 | | | | |
| 182 | cyclo(1S4RAcpc-Nle-4Pal-Pip2c-W-NMeE-N- | E | 2051.65 | C104H | 2049.86 | 94.4 | 1026.7 | [M + 2H] |
| | 4Pal-Trp7Cl-aMeS-Phe4COOH-Y-Sar- | | | 120C1N | | | | 2+ |
| | SbMeW7OMe) | | | 21O22 | | | | |
| 183 | cyclo(ClAc-Nle-4Pal-Morpip6diMe-W- | B | 2087.74 | C104H | 2085.86 | 96.2 | 1044.7 | [M + 2H] |
| | NEtAla-N-4Pal-Trp7Cl-aMeS-Phe4COOH-Y- | | | 124C1N | | | | 2+ |
| | Sar-SbMeW7OMe-C)-NH2 | | | 21O22S | | | | |
| 184 | cyclo(ClAc-Nle-4Pal-Pip2c-W-NMeE-N- | D | 2171.78 | C106H | 2169.86 | 95.2 | 1086.11 | [M + 2H] |
| | Phe4CONH2-Trp7Cl-aMeS-Phe4CONH2- | | | 124C1N | | | | 2+ |
| | Phe3COOH-Sar-SbMeW7OMe-C)-NH2 | | | 23O24S | | | | |
| 185 | cyclo(CLAc-Nle-4Pal-Pipt5OH-W-dProt4OH- | B | 2129.74 | C105H | 2127.84 | 98.0 | 1065.3 | [M + 2H] |
| | N-Phe4COOH-Trp7Cl-aMeS-Phe4CONH2-Y- | | | 122C1N | | | | 2+ |
| | Sar-SbMeW7OMe-C)-NH2 | | | 21O24S | | | | |
| 186 | cyclo(ClAc-Nle-4Pal-Pipt5OH-W-NEtdA-N- | A | 2072.73 | C103H | 2070.86 | 100.0 | 1037.2 | [M + 2H] |
| | 4Pal-Trp7Cl-aMeS-Phe4CONH2-Y-Sar- | | | 123C1N | | | | 2+ |
| | SbMeW7OMe-C)-NH2 | | | 22O21S | | | | |
| 187 | cyclo(bhcLeu-Nle-4Pal-Pipt5OH-W- | H | 2052.67 | C105H | 2050.88 | 99.6 | 2051.8 | [M + 2H] |
| | dProt4OH-alT-4Pal-Trp7Cl-aMeS- | | | 123C1N | | | | + |
| | Phe4CONH2-Phe4COOH-Sar-SbMeW7OMe) | | | 20O22 | | | | |
| 188 | cyclo(bhcLeu-Nle-4Pal-Pipt5OH-W- | H | 2081.67 | C105H | 2079.87 | 95.6 | 2080.8 | [M + 2H] |
| | dProt4OH-N-4Pal-Trp7Cl-aMeS- | | | 122C1N | | | | + |
| | Phe4CONH2-RbOHPhe4COOH-Sar- | | | 21O23 | | | | |
| | SbMeW7OMe) | | | | | | | |
| 189 | cyclo(bhcLeu-Nle-4Pal-Pipt5OH-Trp7az- | H | 2095.70 | C105H | 2093.90 | 98.0 | 1048.4 | [M + 2H] |
| | NMeE-N-4Pal-Trp7Cl-aMeS-Phe4CONH2- | | | 124C1N | | | | 2+ |
| | Phe4CONH2-Sar-SbMeW7OMe) | | | 23O22 | | | | |
| 190 | cyclo(bhcLeu-AlaPent-4Pal-Pip2c-W-NMeE- | H | 2077.68 | C106H | 2075.88 | 98.0 | 1039.8 | [M + 2H] |
| | N-4Pal-Trp7Cl-aMeS-Phe4COOH-Y-Sar- | | | 122C1N | | | | 2+ |
| | SbMeW7OMe) | | | 21O22 | | | | |
| 191 | cyclo(bhcLeu-Nle-4Pal-Pipt5OH-W- | H | 2110.67 | C105H | 2108.86 | 93.9 | 704.3 | [M + 2H] |
| | dProt4OH-N-Phe4COOH-Trp7Cl-aMeS- | | | 121C1N | | | | 3+ |
| | Phe4CONH2-AlaPyrim4COOH-Sar- | | | 22O24 | | | | |
| | SbMeW7OMe) | | | | | | | |
| 192 | cyclo(bhcLeu-Nle-4Pal-Pip2c-W-dProc3OH- | H | 2O23.64 | C103H | 2021.87 | 98.5 | 2022.7 | [M + 2H] |
| | N-4Pal-Trp7Cl-aMeS-Phe4COOH-Y-Sar- | | | 120C1N | | | | + |
| | SbMeW7OMe) | | | 21O21 | | | | |
| 193 | cyclo(bhcLeu-Nle-4Pal-Pipt5OH-Trp7az- | F | 2271.36 | C112H | 2269.95 | 98.8 | 1135.6 | [M + 2H] |
| | dProt4NHC2acidNMe3-N-Phe35F24COOH- | | | 129F4 | | | | 2+ |
| | Trp7CN-aMeS-Phe4CONH2- | | | N24O2 | | | | |
| | Phe35F24COOH-Sar-SbMeW7OMe) | | | 4 | | | | |
| 194 | cyclo(bhcLeu-hSOMe-4Pal-Pipt5OH-W- | H | 2100.25 | C107H | 2098.90 | 92.2 | 2100.1 | [M + 2H] |
| | dProt4OH-N-Phe4COOH-Trp7CN-aMeS- | | | 122N2 | | | | + |
| | Phe4CONH2-Phe4CONH2-Sar- | | | 2O24 | | | | |
| | SbMeW7OMe) | | | | | | | |
| 195 | cyclo(bhcLeu-Nle-4Pal-Pip2c-W-NMeE-N- | G | 2124.78 | C108H | 2122.95 | 80.0 | 1062.9 | [M + 2H] |
| | 4Pal-Trp7Cl-aMeS-Phe4COOH-Me2AEF-Sar- | | | 131C1N | | | | 2+ |
| | SbMeW7OMe) | | | 22O22 | | | | |
| 196 | cyclo(bhcLeu-Nle-4Pal-Pipt5OH-W- | F | 2107.71 | C107H | 2105.89 | 98.0 | 1054.1 | [M + 2H] |
| | dProt4OH-N-Phe4COOH-Trp7Cl-aMeS- | | | 124C1N | | | | 2+ |
| | Phe4CONH2-Phe4CONH2-Sar- | | | 21O23 | | | | |
| | SbMeW7OMe) | | | | | | | |
| 197 | cyclo(bhcLeu-Nle-4Pal-Pip2c-TrpCH2COOH- | E | 2111.70 | C106H | 2109.88 | 93.8 | 1056.4 | [M + 2H] |
| | NMeE-N-4Pal-Trp7Cl-aMeS-Phe4COOH-Y- | | | 124C1N | | | | 2+ |
| | Sar-SbMeW7OMe) | | | 21O24 | | | | |
| 198 | cyclo(bhcLeu-Nle-4Pal-Pip2c-W-NMeE-N- | H | 2071.65 | C104H | 2069.87 | 98.4 | 2070.9 | [M + 2H] |
| | 4Pal2F-Trp7Cl-aMeS-Phe4COOH-Y-Sar- | | | 121CIF | | | | + |
| | SbMeW7OMe) | | | N21O22 | | | | |
| 199 | cyclo(bhcLeu-Nle-4Pal-Pip2c-W-NMeE-N- | H | 2089.72 | C103H | 2087.84 | 97.4 | 696.8 | [M + 2H] |
| | 4Pal-Trp7Cl-aMeS-F4SO3H-Y-Sar- | | | 122C1N | | | | 3+ |
| | SbMeW7OMe) | | | 21O23S | | | | |
| 200 | cyclo(bhcLeu-Ahp-4Pal-Pip2c-W-dProt4OH- | E | 2041.27 | C106H | 2039.95 | 99.6 | 1021.6 | [M + 2H] |
| | N-4Pal-Trp7CN-aMeS-Phe4CONH2-TyrOMe- | | | 125N2 | | | | 2+ |
| | Sar-SbMeW7OMe) | | | 3O20 | | | | |
| 201 | cyclo(bhcLeu-Nle-4Pal2F-Pip2c-W-NMeE-N- | H | 2071.65 | C104H | 2069.87 | 92.0 | 1036.3 | [M + 2H] |
| | 4Pal-Trp7Cl-aMeS-Phe4COOH-Y-Sar- | | | 121CIF | | | | 2+ |
| | SbMeW7OMe) | | | N21O22 | | | | |
| 202 | cyclo(bhcLeu-Nle-4Pal-Pip-W-CeG-N-4Pal- | F | 2065.67 | C105H | 2063.88 | 98.0 | 1033.6 | [M + 2H] |
| | Trp7Cl-aMeS-Phe4COOH-Phe3CONH2-Sar- | | | 122C1N | | | | 2+ |
| | SbMeW7OMe) | | | 21O22 | | | | |
| 203 | cyclo(ClAc-Nle-4Pal-Pipt5OH-W-dP-N-4Pal- | B | 2071.70 | C103H | 2069.83 | 100.0 | 1036.7 | [M + 2H] |
| | Trp7Cl-aMeS-Phe4COOH-Y-Sar- | | | 120C1N | | | | 2+ |
| | SbMeW7OMe-C)-NH2 | | | 21O22S | | | | |
| 204 | cyclo(bhcLeu-Nle-4Pal-Pipt5OH-3Inda- | F | 2194.34 | C108H | 2192.87 | 90.8 | 1097.9 | [M + 2H] |
| | dProt40AcOH-N-Phe4COOH-Trp7CN-aMeS- | | | 124N2 | | | | 2+ |
| | F4SO2NH2-Phe4COOH-Sar-SbMeW7OMe) | | | 2O27S | | | | |
| 205 | cyclo(ClAc-Nle-Phe4CH2NH2-S-Trp7az- | A | 2075.69 | C101H | 2073.84 | 100.0 | 1038.7 | [M + 2H] |
| | NMeE-N-4Pal-Trp7Cl-aMeS-Phe3CONH2-Y- | | | 120C1N | | | | 2+ |
| | Sar-aMeW-C)-NH2 | | | 23O22S | | | | |
| 206 | cyclo(CLAc-Cit-Phe4CH2NH2-S-SbMeW-dP- | D | 2105.37 | C107H | 2103.95 | 76.7 | 2105.0 | [M + 2H] |
| | N-Phe4CH2NH2-2Nal-aMeS-Phe4COOH- | | | 129N2 | | | | + |
| | Phe4CH2NH2-Sar-SbMeW-C)-NH2 | | | 3O21S | | | | |
| 207 | cyclo(bhcLeu-Nle-4Pal-Pipt5OH-W- | E | 2168.15 | C108H | 2165.83 | 99.0 | 1084.5 | [M + 2H] |
| | dProt4OH-N-Phe3C14COOH-Trp7CN-aMeS- | | | 121C12 | | | | 2+ |
| | Phe4CONH2-Phe3C14COOH-Sar- | | | N21O24 | | | | |
| | SbMeW7OMe) | | | | | | | |
| 208 | cyclo(ClAc-Nle-4Pal-Pip-W-NMeE-N-4Pal- | A | 2085.73 | C104H | 2083.85 | 100.0 | 1043.7 | [M + 2H] |
| | Trp7Cl-aMeS-Phe4COOH-Y-Sar- | | | 122C1N | | | | 2+ |
| | SbMeW7Me-C)-NH2 | | | 21O22S | | | | |
| 209 | cyclo(CLAc-Nle-4Pal-NMeS-Trp7az-NMeE-N- | B | 2134.71 | C103H | 2132.83 | 74.0 | 1068.2 | [M + 2H] |
| | Phe4COOH-Trp7Cl-aMeS-Phe4CONH2-Y- | | | 121C1N | | | | 2+ |
| | Sar-SbMeW40Me-C)-NH2 | | | 22O25S | | | | |
| 210 | cyclo(bhcLeu-Nle-4Pal-Pipt5OH- | H | 2208.36 | C109H | 2206.89 | 89.0 | 2207.9 | [M + 2H] |
| | TrpCH2COOH-dProt4OH-N-Phe4COOH- | | | 126N2 | | | | + |
| | Trp7CN-aMeS-F4SO2NH2- | | | 2O27S | | | | |
| | RbOHPhe4CONH2-Sar-SbMeW7OMe) | | | | | | | |
| 211 | cyclo(ClAc-Nle-4Pal-RbOHPhe-Trp7az- | A | 2154.75 | C106H | 2152.83 | 100.0 | 1078.2 | [M + 2H] |
| | NMeE-N-4Pal-Trp7Cl-aMeS-Phe4COOH-Y- | | | 121C1N | | | | 2+ |
| | Sar-SbMeW7OMe-C)-NH2 | | | 22O24S | | | | |
| 212 | cyclo(ClAc-Nle-4Pal-Pip-W-NMeE-N- | A | 2158.77 | C107H | 2156.85 | 98.0 | 1080.2 | [M + 2H] |
| | F4MCO2H-Trp7Cl-aMeS-Phe4COOH-Y-Sar- | | | 125C1N | | | | 2+ |
| | SbMeW7OMe-C)-NH2 | | | 20O25S | | | | |
| 213 | cyclo(bhcLeu-AhpF3-4Pal-Pip2c-W-NMeE-N- | H | 2121.66 | C105H | 2119.86 | 99.1 | 1061.4 | [M + 2H] |
| | 4Pal-Trp7Cl-aMeS-Phe4COOH-Y-Sar- | | | 121CIF | | | | 2+ |
| | SbMeW7OMe) | | | 3N21O | | | | |
| | | | | 22 | | | | |
| 214 | cyclo(dbhcLeu-Nle-4Pal-Morpip6diMe-W- | G | 2081.71 | C106H | 2079.91 | 94.1 | 1041.9 | [M + 2H] |
| | NMeE-N-4Pal-Trp7Cl-aMeS-Phe4CONH2-Y- | | | 126C1N | | | | 2+ |
| | Sar-SbMeW7OMe) | | | 21O22 | | | | |
| 215 | cyclo(ClAc-Cit-Phe4CH2NH2-S-SbMeW-dP- | A | 2104.69 | C101H | 2102.83 | 98.1 | 1053.0 | [M + 2H] |
| | N-Phe4COOH-Trp7Cl-aMeS-Phe4COOH-H- | | | 119C1N | | | | 2+ |
| | Sar-SbMeW-C)-NH2 | | | 24O23S | | | | |
| 216 | cyclo(CLAc-Nle-4Pal-Pip2c-W-NEtCONH2G- | D | 2087.70 | C102H | 2085.84 | 97.6 | 1043.65 | [M + 2H] |
| | N-4Pal-Trp7Cl-aMeS-Phe4COOH-Y-Sar- | | | 120C1N | | | | 2+ |
| | SbMeW7OMe-C)-NH2 | | | 23O22S | | | | |
| 217 | cyclo(ClAc-Nle-H-Bhv-Trp7az-NMeE-N- | A | 2095.68 | C100H | 2093.83 | 92.7 | 1048.8 | [M + 2H] |
| | 4Pal-Trp7Cl-aMeS-Phe4COOH-Y-Sar- | | | 120C1N | | | | 2+ |
| | SbMeW7OMe-C)-NH2 | | | 23O24S | | | | |
| 218 | cyclo(ClAc-Nle-4Pal-Pip2c-W-NMeE-N- | C | 2131.75 | C105H | 2129.85 | 86.5 | 1066.3 | [M + 2H] |
| | TyrOMe-Trp7Cl-aMeS-Phe4COOH-Y-Sar- | | | 124C1N | | | | 2+ |
| | SbMeW7OMe-C)-NH2 | | | 21O24S | | | | |
| 219 | cyclo(ClAc-Nle-4Pal-Pip2c-Trp7az- | A | 2071.71 | C101H | 2069.86 | 87.0 | 1036.7 | [M + 2H] |
| | dProc4NH2-N-4Pal-Trp7Cl-aMeS- | | | 120C1N | | | | 2+ |
| | Phe4CONH2-Y-Sar-SbMeW7OMe-C)-NH2 | | | 25O20S | | | | |
| 220 | cyclo(ClAc-Nle-4Pal-Pip-Trp7az-CeG-N-4Pal- | A | 2088.69 | C102H | 2086.82 | 93.0 | 1045.2 | [M + 2H] |
| | Trp7Cl-aMeS-Phe4COOH-Y-Sar- | | | 119C1N | | | | 2+ |
| | SbMeW7OMe-C)-NH2 | | | 22O23S | | | | |
| 221 | cyclo(bhcLeu-Nle-4Pal-Pip-W-NMeE-N-4Pal- | G | 2079.70 | C106H | 2077.89 | 97.7 | 1040.4 | [M + 2H] |
| | Trp7Cl-aMeS-Phe4COOH-Phe3CONH2-Sar- | | | 124C1N | | | | 2+ |
| | SbMeW7OMe) | | | 21O22 | | | | |
| 222 | cyclo(CLAc-AlaPent-mAF-S-Trp7Cl-dP- | A | 2178.64 | C106H | 2175.77 | 98.3 | 1089.5 | [M + 2H] |
| | NMeN-Phe4COOH-Trp7Cl-aMeS- | | | 120C13 | | | | 2+ |
| | Phe4COOH-mAF-Sar-Trp4Cl-C)-NH2 | | | N21O2 | | | | |
| | | | | 2S | | | | |
| 223 | cyclo(CIAc-Nle-4Pal-S-Trp7az-NMeE-N-4Pal- | A | 2048.62 | C99H1 | 2046.79 | 97.2 | 1025.1 | [M + 2H] |
| | Trp7Cl-aMeS-Phe4COOH-Y-Sar-Trp1Me-C)- | | | 15C1N2 | | | | 2+ |
| | NH2 | | | 2O23S | | | | |
| 224 | cyclo(ClAc-Nle-Phe4CH2NH2-S-Trp7az- | A | 2090.70 | C102H | 2088.84 | 95.0 | 1046.0 | [M + 2H] |
| | NMeE-N-4Pal-Trp7Cl-aMeS-F4MCO2H-Y- | | | 121C1N | | | | 2+ |
| | Sar-aMeW-C)-NH2 | | | 22O23S | | | | |
| 225 | cyclo(bhcLeu-Nle-4Pal-Pip2c-W-NMeE- | H | 2068.68 | C104H | 2066.89 | 94.0 | 1034.9 | [M + 2H] |
| | DapUrea-4Pal-Trp7Cl-aMeS-Phe4COOH-Y- | | | 123C1N | | | | 2+ |
| | Sar-SbMeW7OMe) | | | 22O22 | | | | |
| 226 | cyclo(ClAc-Ahp-Y-AlaPiperaz-SbMeW-dP-N- | D | 2097.78 | C105H | 2095.90 | 94.0 | 1039.3 | [M + 2H] |
| | 4Pal-Trp7Cl-aMeS-Phe4COOH-3Pal-Sar- | | | 126C1N | | | | 2+ |
| | aMeW-C)-NH2 | | | 23O20S | | | | |
| 227 | cyclo(ClAc-K-4Pal-S-1Nal-NMeA-N-4Pal- | D | 1978.19 | C101H | 1976.83 | 92.0 | 660.3 | [M + 2H] |
| | 2Nal-aMeS-Phe4COOH-Y-Sar-W-C)-NH2 | | | 116N2 | | | | 3+ |
| | | | | 0O21S | | | | |
| 228 | cyclo(ClAc-Nle-4Pal-Pip-Trp7az-NEtCNG-N- | I | 2069.69 | C102H | 2067.83 | 93.5 | 1035.6 | [M + 2H] |
| | 4Pal-Trp7Cl-aMeS-Phe4COOH-Y-Sar- | | | 118C1N | | | | 2+ |
| | SbMeW7OMe-C)-NH2 | | | 23O21S | | | | |
| 229 | cyclo(bhcLeu-Nle-4Pal-Pipt5OH-W-NMeE-N- | F | 2094.71 | C106H | 2092.90 | 99.8 | 1047.8 | [M + 2H] |
| | 4Pal-Trp7Cl-aMeS-Phe4CONH2- | | | 125C1N | | | | 2+ |
| | Phe4CONH2-Sar-SbMeW7OMe) | | | 22O22 | | | | |
| 230 | cyclo(cHxc1acid4NH2-Nle-4Pal-Pip2c-W- | G | 2067.69 | C105H | 2065.89 | 99.0 | 1034.7 | [M + 2H] |
| | NMeE-N-4Pal-Trp7Cl-aMeS-Phe4COOH-Y- | | | 124C1N | | | | 2+ |
| | Sar-SbMeW7OMe) | | | 21O22 | | | | |
| 231 | cyclo(ClAc-Nle-4Pal-NMeDab-Trp7az-NMeE- | A | 2075.69 | C101H | 2073.84 | 95.0 | 1038.72 | [M + 2H] |
| | N-4Pal-Trp7Cl-aMeS-Phe4COOH-Y-Sar- | | | 120C1N | | | | 2+ |
| | aMeW-C)-NH2 | | | 23O22S | | | | |
| 232 | cyclo(CLAc-AlaPent-4Pal-S-Trp7Cl-NEtAla- | A | 2058.62 | C100H | 2056.78 | 95.4 | 1030.1 | [M + 2H] |
| | N-Phe4COOH-Trp7az-aMeS-Phe4COOH-Y- | | | 113C1N | | | | 2+ |
| | Sar-Trp7az-C)-NH2 | | | 22O23S | | | | |
| 233 | cyclo(CLAc-hF-4Pal-Dap-W-dP-N-4Pal-2Nal- | A | 1996.21 | C103H | 1994.84 | 98.0 | 998.8 | [M + 2H] |
| | aMeS-Phe4COOH-3Pal-Sar-W-C)-NH2 | | | 114N2 | | | | 2+ |
| | | | | 2O19S | | | | |
| 234 | cyclo(bhcLeu-Ahp-SbMe4Pal-T-W- | E | 2100.29 | C108H | 2098.94 | 96.0 | 1051.0 | [M + 2H] |
| | dProt4OH-N-Phe4COOH-Trp7CN-aMeS- | | | 126N2 | | | | 2+ |
| | Phe4CONH2-Phe4CONH2-Sar- | | | 2O23 | | | | |
| | SbMeW7OMe) | | | | | | | |
| 235 | cyclo(CLAc-Nle-4Pal-DapMe-Trp7az-NMeE- | A | 2061.67 | C100H | 2059.82 | 94.0 | 1031.6 | [M + 2H] |
| | N-4Pal-Trp7Cl-aMeS-Phe4COOH-Y-Sar- | | | 118C1N | | | | 2+ |
| | aMeW-C)-NH2 | | | 23O22S | | | | |
| 236 | cyclo(ClAc-Nle-4Pal-Pip2c-W-dP-N-Furan- | A | 2045.66 | C101H | 2043.82 | 100.0 | 1023.6 | [M + 2H] |
| | Trp7Cl-aMeS-Phe4COOH-Y-Sar- | | | 118C1N | | | | 2+ |
| | SbMeW7OMe-C)-NH2 | | | 21O22S | | | | |
| 237 | cyclo(ClAc-Ahp-4Pal-S-SbMeW-dP-N- | D | 2O23.22 | C102H | 2021.86 | 91.0 | 1012.0 | [M + 2H] |
| | Phe4COOH-2Nal-aMeS-Phe4COOH-H-Sar- | | | 119N2 | | | | 2+ |
| | aMeW-C)-NH2 | | | 1O22S | | | | |
| 238 | cyclo(bhcLeu-LysMe2-4Pal-Pipt5OH-W- | F | 2110.76 | C107H | 2108.93 | 100.0 | 1055.6 | [M + 2H] |
| | NMeE-N-4Pal-Trp7Cl-aMeS-Phe4CONH2-Y- | | | 129C1N | | | | 2+ |
| | Sar-SbMeW7OMe) | | | 22O22 | | | | |
| 239 | cyclo(ClAc-Q-4Pal-S-Trp7az-NMeE-N-4Pal- | A | 2084.01 | C97H1 | 2081.71 | 100.0 | 1042.7 | [M + 2H] |
| | Trp7Cl-aMeS-Phe4COOH-Y-Sar-Trp4Cl-C)- | | | 09C12N | | | | 2+ |
| | NH2 | | | 23O24S | | | | |
| 240 | cyclo(bhcLeu-Nle-4Pal-Pip2c-W-NMeE-Q- | H | 2123.75 | C108H | 2121.92 | 98.2 | 1061.8 | [M + 2H] |
| | Phe4CONH2-Trp7Cl-aMeS-Phe4COOH- | | | 128C1N | | | | 2+ |
| | TyrOMe-Sar-SbMeW7OMe) | | | 21O23 | | | | |
| 241 | cyclo(bhcLeu-Nle-40Me3Pal-Pip2c-W- | H | 2083.69 | C105H | 2081.89 | 99.7 | 1042.5 | [M + 2H] |
| | NMeE-N-4Pal-Trp7Cl-aMeS-Phe4COOH-Y- | | | 124C1N | | | | 2+ |
| | Sar-SbMeW7OMe) | | | 21O23 | | | | |
| 242 | cyclo(ClAc-K-Y-S-W-dP-Q-4Pal-W-aMeS- | D | 1997.19 | C100H | 1995.84 | 90.0 | 999.8 | [M + 2H] |
| | Phe4COOH-Y-Sar-W-C)-NH2 | | | 117N2 | | | | 2+ |
| | | | | 1O22S | | | | |
| 243 | cyclo(CIAc-Nle-RbMeY-S-Trp7az-NMeE-N- | A | 2107.69 | C102H | 2105.82 | 98.4 | 1054.7 | [M + 2H] |
| | 4Pal-Trp7Cl-aMeS-Phe4COOH-Y-Sar- | | | 120C1N | | | | 2+ |
| | SbMeW7OMe-C)-NH2 | | | 21O25S | | | | |
| 244 | cyclo(cHxc1acid4NH2-Nle-Phe4CH2NH2-S- | G | 2070.69 | C105H | 2068.89 | 97.4 | 1036.5 | [M + 2H] |
| | W-NMeE-N-4Pal-Trp7Cl-aMeS-Phe4COOH- | | | 125C1N | | | | 2+ |
| | Y-Sar-SbMeW7OMe) | | | 20O23 | | | | |
| 245 | cyclo(ClAc-LysN3-4Pal-Dap-SbMeW-dP-N- | D | 2068.71 | C101H | 2066.86 | 76.0 | 689.9 | [M + 2H] |
| | 4Pal-Trp7Cl-aMeS-Phe4COOH- | | | 119C1N | | | | 3+ |
| | Phe4CH2NH2-Sar-aMeW-C)-NH2 | | | 26O19S | | | | |
| 246 | cyclo(2SBrBu-Nle-4Pal-Spip-W-NMeE-N- | A | 2146.83 | C105H | 2144.85 | 98.9 | 1072.7 | [M + 2H] |
| | 4Pal-Trp7Cl-aMeS-Phe4CONH2-Y-Sar- | | | 125C1N | | | | 2+ |
| | SbMeW7OMe-C)-NH2 | | | 22O22 | | | | |
| | | | | S2 | | | | |
| 247 | cyclo(bhcLeu-Nle-4Pal-Pipt5OH-W- | F | 2108.69 | C107H | 2106.87 | 98.0 | 1055.5 | [M + 2H] |
| | dProt4OH-N-Phe4COOH-Trp7Cl-aMeS- | | | 123C1N | | | | 2+ |
| | Phe4CONH2-Phe4COOH-Sar-SbMeW7OMe) | | | 20O24 | | | | |
| 248 | cyclo(1R4SAcpc-hF-4Pal-S-W-NMeA-N- | G | 1938.10 | C102H | 1936.84 | 82.0 | 970.2 | [M + 2H] |
| | 4Pal-W-aMeS-Phe4COOH-Y-Sar-W) | | | 112N2 | | | | 2+ |
| | | | | 0O20 | | | | |
| 249 | cyclo(ClAc-Nle-4Pal-Pip2c-W-NMeE-N-4Pal- | D | 2102.71 | C103H | 2100.84 | 96.9 | 2101.86 | [M + 2H] |
| | Trp7Cl-aMeS-Phe3COOH-Y-Sar- | | | 121C1N | | | | 1+ |
| | SbMeW7OMe-C)-NH2 | | | 22O23S | | | | |
| 250 | cyclo(2SBrPr-Nle-Phe4CH2NH2-S-Trp7az- | A | 2119.74 | C103H | 2117.86 | 90.0 | 1060.6 | [M + 2H] |
| | NMeE-N-4Pal-Trp7Cl-aMeS-Phe4CONH2-Y- | | | 124C1N | | | | 2+ |
| | Sar-SbMeW7OMe-C)-NH2 | | | 23O23S | | | | |
| 251 | cyclo(ClAc-Nle-4Pal-Pip2c-W-NMeE-N-4Pal- | D | 2130.72 | C104H | 2128.83 | 97.8 | 1065.6 | [M + 2H] |
| | Trp7Cl-aMeS-Phe4COOH-Phe4COOH-Sar- | | | 121C1N | | | | 2+ |
| | SbMeW7OMe-C)-NH2 | | | 22O24S | | | | |
| 252 | cyclo(CLAc-Nle-Phe4CH2NH2-S-W-NMeE- | D | 2119.74 | C104H | 2117.85 | 98.0 | 1060.3 | [M + 2H] |
| | N-SbMe4Pal-Trp7Cl-aMeS-Phe4COOH-Y- | | | 124C1N | | | | 2+ |
| | Sar-SbMeW7OMe-C)-NH2 | | | 21O24S | | | | |
| 253 | cyclo(ClAc-Nle-4Pal-Tza-Trp7az-NMeE-N- | A | 2145.76 | C103H | 2143.79 | 100.0 | 1073.7 | [M + 2H] |
| | 4Pal-Trp7Cl-aMeS-Phe4COOH-Y-Sar- | | | 118C1N | | | | 2+ |
| | SbMeW7OMe-C)-NH2 | | | 23O23 | | | | |
| | | | | S2 | | | | |
| 254 | cyclo(bhcLeu-Nle-4Pal-Pipt5OH-Trp7az- | E | 2099.26 | C107H | 2097.92 | 98.3 | 1050.5 | [M + 2H] |
| | dProt4OH-N-Phe4COOH-Trp7CN-aMeS- | | | 123N2 | | | | 2+ |
| | Phe4CONH2-Phe4CONH2-Sar- | | | 3O23 | | | | |
| | SbMeW7OMe) | | | | | | | |
| 255 | cyclo(ClAc-Nle-4Pal-Pip2c-Trp7az-NMeE-N- | A | 2418.98 | C114H | 2416.94 | 99.0 | 1209.8 | [M + 2H] |
| | 4Pal-Trp7Cl-aMeS-Phe4COOH-Y-Sar- | | | 137C1N | | | | 2+ |
| | SbMeW7OMe-C-G-E-E-NH2 | | | 26O30S | | | | |
| 256 | cyclo(CIAc-K-Y-S-W-dP-Cit-4Pal-W-aMeS- | D | 2026.23 | C101H | 2O24.87 | 90.0 | 676.4 | [M + 2H] |
| | Phe4COOH-Y-Sar-W-C)-NH2 | | | 120N2 | | | | 3+ |
| | | | | 2O22S | | | | |
| 257 | cyclo(bhcLeu-Nle-4Pal-Pipt5OH-Trp7az- | F | 2238.46 | C111H | 2236.96 | 95.3 | 1119.2 | [M + 2H] |
| | dProt4NMe3-N-TyrOcm-Trp7CN-aMeS- | | | 134N2 | | | | + |
| | F4SO2NH2-TyrOcm-Sar-SbMeW7OMe) | | | 3O26S | | | | |
| 258 | cyclo(ClAc-Nle-Phe4CH2NH2-S-Trp7az- | A | 2063.68 | C101H | 2061.83 | 90.0 | 1032.4 | [M + 2H] |
| | NMeE-N-4Pal-Trp7Cl-aMeS-Phe4COOH-Y- | | | 120C1N | | | | 2+ |
| | Sar-SbMeW7OMe-Cysteamine) | | | 21O23S | | | | |
| 259 | cyclo(ClAc-Nle-RbOHPhe-S-Trp7az-NMeE- | A | 2093.66 | C101H | 2091.80 | 100.0 | 1047.6 | [M + 2H] |
| | N-4Pal-Trp7Cl-aMeS-Phe4COOH-Y-Sar- | | | 118C1N | | | | 2+ |
| | SbMeW7OMe-C)-NH2 | | | 21O25S | | | | |
| 260 | cyclo(CLAc-hF-Y-S-W-NMeA-N-4Pal-W- | A | 1997.19 | C99H1 | 1995.85 | 94.2 | 999.3 | [M + 2H] |
| | aMeS-Phe4COOH-R-Sar-W-C)-NH2 | | | 17N23 | | | | 2+ |
| | | | | O21S | | | | |
| 261 | cyclo(ClAc-Nle-4Pal-Pip2c-Trp7az- | A | 2073.68 | C101H | 2071.82 | 100.0 | 691.8 | [M + 2H] |
| | dProc4OH-N-4Pal-Trp7Cl-aMeS-Phe4COOH- | | | 118C1N | | | | 3+ |
| | Y-Sar-SbMeW7OMe-C)-NH2 | | | 23O22S | | | | |
| 262 | cyclo(bhcLeu-Nle-4Pal-Pip2c-Trp7Cl-NMeE- | H | 2088.11 | C104H | 2085.84 | 97.2 | 1043.8 | [M + 2H] |
| | N-4Pal-Trp7Cl-aMeS-Phe4COOH-Y-Sar- | | | 121C12 | | | | 2+ |
| | SbMeW7OMe) | | | N21O22 | | | | |
| 263 | cyclo(CLAc-hF-Y-S-Trp7F-NMeA-N-4Pal-W- | A | 2022.17 | C102H | 2020.80 | 100.0 | 1011.8 | [M + 2H] |
| | aMeS-Phe4COOH-Y-Sar-W-C)-NH2 | | | 113FN | | | | 2+ |
| | | | | 20O22S | | | | |
| 264 | cyclo(ClAc-Nle-H-Pip2c-Trp7az-NMeE-N- | A | 2092.68 | C100H | 2090.83 | 98.1 | 1047.2 | [M + 2H] |
| | 4Pal-Trp7Cl-aMeS-Phe4COOH-Y-Sar- | | | 119C1N | | | | 2+ |
| | SbMeW7OMe-C)-NH2 | | | 24O23S | | | | |
| 265 | cyclo(CLAc-hF-Y-S-1Inda-NMeA-N-4Pal-W- | A | 2005.20 | C101H | 2003.81 | 94.2 | 1003.5 | [M + 2H] |
| | aMeS-Phe4COOH-Y-Sar-W-C)-NH2 | | | 113N2 | | | | 2+ |
| | | | | 1O22S | | | | |
| 266 | cyclo(bhcLeu-Nle-4Pal-Pip2c-W-dPro44F2-N- | H | 2043.62 | C103H | 2041.85 | 94.5 | 2043.3 | [M + 2H] |
| | 4Pal-Trp7Cl-aMeS-Phe4COOH-Y-Sar- | | | 118CIF | | | | + |
| | SbMeW7OMe) | | | 2N21O | | | | |
| | | | | 20 | | | | |
| 267 | cyclo(dbhcLeu-Nle-4Pal-Pipt5OH-W-NMeE- | G | 2110.71 | C107H | 2108.89 | 96.1 | 704.4 | [M + 2H] |
| | N-Phe4COOH-Trp7Cl-aMeS-Phe4CONH2-Y- | | | 125C1N | | | | 3+ |
| | Sar-SbMeW7OMe) | | | 20O24 | | | | |
| 268 | cyclo(c3amCb1c-Nle-4Pal-Pip2c-W-NMeE-N- | G | 2053.66 | C104H | 2051.88 | 93.2 | 1O27.7 | [M + 2H] |
| | 4Pal-Trp7Cl-aMeS-Phe4COOH-Y-Sar- | | | 122C1N | | | | 2+ |
| | SbMeW7OMe) | | | 21O22 | | | | |
| 269 | cyclo(bhcLeu-Nle-4Pal-Pip2c-W-NMeE-N- | E | 2066.70 | C105H | 2064.91 | 90.0 | 689.5 | [M + 2H] |
| | 4Pal-Trp7Cl-aMeS-Phe4COOH- | | | 125C1N | | | | 3+ |
| | Phe4CH2NH2-Sar-SbMeW7OMe) | | | 22O21 | | | | |
| 270 | cyclo(CLAc-Nle-4Pal-NMeS-W-NMeE-N- | D | 2118.71 | C103H | 2116.83 | 94.0 | 1059.31 | [M + 2H] |
| | 4Pal-Trp7Cl-aMeS-Phe4COOH-Phe3CONH2- | | | 121C1N | | | | 2+ |
| | Sar-SbMeW7OMe-C)-NH2 | | | 22O24S | | | | |
| 271 | cyclo(ClAc-Nle-4Pal-Pipc4NH2-W-NMeE-N- | A | 2116.74 | C104H | 2114.85 | 100.0 | 1059.2 | [M + 2H] |
| | 4Pal-Trp7Cl-aMeS-Phe4COOH-Y-Sar- | | | 123C1N | | | | 2+ |
| | SbMeW7OMe-C)-NH2 | | | 22O23S | | | | |
| 272 | cyclo(ClAc-Nle-4Pal-Pipt5Me-W-NMeE-N- | A | 2115.75 | C105H | 2113.86 | 100.0 | 1058.6 | [M + 2H] |
| | 4Pal-Trp7Cl-aMeS-Phe4COOH-Y-Sar- | | | 124C1N | | | | 2+ |
| | SbMeW7OMe-C)-NH2 | | | 21O23S | | | | |
| 273 | cyclo(2SBrPr-Nle-4Pal-Bav-Trp7az-NMeE-N- | A | 2118.76 | C103H | 2116.88 | 98.0 | 1060.3 | [M + 2H] |
| | 4Pal-Trp7Cl-aMeS-Phe4CONH2-Y-Sar- | | | 125C1N | | | | 2+ |
| | SbMeW7OMe-C)-NH2 | | | 24O22S | | | | |
| 274 | cyclo(SBrPh-Nle-4Pal-S-Trp7az-NMeE-N- | A | 2124.72 | C105H | 2122.82 | 98.6 | 1062.6 | [M + 2H] |
| | 4Pal-Trp7Cl-aMeS-Phe4COOH-Y-Sar-aMeW- | | | 119C1N | | | | 2+ |
| | C)-NH2 | | | 22O23S | | | | |
| 275 | cyclo(bhcLeu-Nle3F2-4Pal-Pip2c-W-NMeE- | F | 2089.64 | C104H | 2087.86 | 90.0 | 1045.7 | [M + 2H] |
| | N-4Pal-Trp7Cl-aMeS-Phe4COOH-Y-Sar- | | | 120CIF | | | | 2+ |
| | SbMeW7OMe) | | | 2N21O | | | | |
| | | | | 22 | | | | |
| 276 | cyclo(ClAc-Nle-4Pal-Pip2c-W-dP-N-4Pal- | A | 2070.72 | C103H | 2068.85 | 97.4 | 1036.3 | [M + 2H] |
| | Trp7Cl-aMeS-Phe4COOH-Y-dNMeA- | | | 121C1N | | | | 2+ |
| | SbMeW7OMe-C)-NH2 | | | 22O21S | | | | |
| 277 | cyclo(ClAc-Nle-4Pal-Dab-Trp7az-NMeE-N- | A | 2061.67 | C100H | 2059.82 | 99.4 | 1031.8 | [M + 2H] |
| | 4Pal-Trp7Cl-aMeS-Phe4COOH-Y-Sar-aMeW- | | | 118C1N | | | | 2+ |
| | C)-NH2 | | | 23O22S | | | | |
| 278 | cyclo(CLAc-Q-4Pal-S-4Quin-dP-N-4Pal- | A | 2041.59 | C99H1 | 2039.76 | 96.0 | 1021.6 | [M + 2H] |
| | Trp7az-aMeS-Phe4COOH-Y-P-Trp4Cl-C)- | | | 10C1N2 | | | | 2+ |
| | NH2 | | | 3O22S | | | | |
| 279 | cyclo(ClAc-Nle-4Pal-Pip2c-W-dProt4F-N- | A | 2074.68 | C102H | 2072.82 | 100.0 | 1037.9 | [M + 2H] |
| | 4Pal-Trp7Cl-aMeS-Phe4COOH-Y-Sar- | | | 118CIF | | | | 2+ |
| | SbMeW7OMe-C)-NH2 | | | N22O2 | | | | |
| | | | | 1S | | | | |
| 280 | cyclo(bhcLeu-Ahp-SbMe4Pal-E-W- | E | 2128.30 | C109H | 2126.93 | 100.0 | 1065.0 | [M + 2H] |
| | dProt4OH-N-Phe4COOH-Trp7CN-aMeS- | | | 126N2 | | | | 2+ |
| | Phe4CONH2-Phe4CONH2-Sar- | | | 2O24 | | | | |
| | SbMeW7OMe) | | | | | | | |
| 281 | cyclo(ClAc-Nle-4Pal-cPrS-Trp7az-NMeE-N- | A | 2074.66 | C101H | 2072.81 | 97.0 | 1037.8 | [M + 2H] |
| | 4Pal-Trp7Cl-aMeS-Phe4COOH-Y-Sar-aMeW- | | | 117C1N | | | | 2+ |
| | C)-NH2 | | | 22O23S | | | | |
| 282 | cyclo(bhcLeu-Ahp-SbMe4Pal-hE-W- | E | 2142.33 | C110H | 2140.95 | 98.5 | 1071.9 | [M + 2H] |
| | dProt4OH-N-Phe4COOH-Trp7CN-aMeS- | | | 128N2 | | | | 2+ |
| | Phe4CONH2-Phe4CONH2-Sar- | | | 2O24 | | | | |
| | SbMeW7OMe) | | | | | | | |
| 283 | cyclo(ClAc-Nle-Phe4CH2NH2-S-Trp7az- | A | 2106.70 | C102H | 2104.83 | 94.3 | 1054.1 | [M + 2H] |
| | NMeE-N-4Pal-Trp7Cl-aMeS-TyrOcm-Y-Sar- | | | 121C1N | | | | 2+ |
| | aMeW-C)-NH2 | | | 22O24S | | | | |
| 284 | cyclo(bhcLeu-Nle-4Pal-Pipt5OH-Trp7az- | F | 2158.28 | C109H | 2156.91 | 100.0 | 1079.7 | [M + 2H] |
| | dProt40AcOH-N-Phe4COOH-Trp7CN-aMeS- | | | 124N2 | | | | 2+ |
| | Phe4CONH2-Phe4COOH-Sar-SbMeW7OMe) | | | 2O26 | | | | |
| 285 | cyclo(bhcLeu-Nle-4Pal-Pipt5OH-W- | E | 2159.31 | C110H | 2157.93 | 100.0 | 1080.4 | [M + 2H] |
| | dProt4OH-N-TyrOcm-Trp7CN-aMeS- | | | 127N2 | | | | 2+ |
| | Phe4CONH2-TyrOcm-Sar-SbMeW7OMe) | | | 1O26 | | | | |
| 286 | cyclo(bhcLeu-Nle-4Pal-Pipt5OH-1Inda- | E | 2196.35 | C108H | 2194.89 | 98.0 | 1098.9 | [M + 2H] |
| | dProt4OH-N-TyrOcm-Trp7CN-aMeS- | | | 126N2 | | | | 2+ |
| | F4SO2NH2-TyrOcm-Sar-SbMeW7OMe) | | | 2O27S | | | | |
| 287 | cyclo(bhcLeu-Nle-4Pal-Pip2c-Trp7az- | E | 11.00 | C108H | 2126.94 | 90.0 | 1064.8 | [M + 2H] |
| | dProt4NMe2-N-Phe4COOH-Trp7CN-aMeS- | | | 126N2 | | | | 2+ |
| | Phe4COOH-RbOHPhe4CONH2-Sar- | | | 4O23 | | | | |
| | SbMeW7OMe) | | | | | | | |
| 288 | cyclo(bhcLeu-Nle-4Pal-Pip2c-W-NMeE-N- | G | 2096.68 | C106H | 2094.87 | 100.0 | 2097.0 | [M + 2H] |
| | Phe4COOH-Trp7Cl-aMeS-Phe4COOH-Y-Sar- | | | 123C1N | | | | + |
| | SbMeW7OMe) | | | 20O24 | | | | |
| 289 | cyclo(CLAc-F-Y-S-W-Sar-N-Y-W-S-Y-Y-Sar- | A | 2264.38 | C111H | 2262.88 | 95.0 | 1132.9 | [M + 2H] |
| | W-C)-G-E-E-NH2 | | | 126N2 | | | | 2+ |
| | | | | 2O29S | | | | |

TABLE 2
Biological Activities of Example Nos. 1-289 (SEQ ID NOS. 1-289)

| | | | SPR | SPR | |
|---|---|---|---|---|---|
| SEQ | TR-FRET, | TR-FRET | hTNFR1 | hTNFR2 | THP-1 |
| ID NO. | IC₅₀ (nM) | K_{d}^{app} (nM) | K_{D} (nM) | K_{D} (nM) | IC₅₀ (nM) |
| 1 | NA | NA | NA | NA | 226.7 |
| 2 | | 0.07 | 0.07 | >1000 | 0.5 |
| 3 | | 0.11 | 0.04 | >1000 | 0.5 |
| 4 | | 0.09 | 0.10 | >1000 | 0.5 |
| 5 | 0.20 | 0.16 | 0.10 | >1000 | 1.2 |
| 6 | | 0.08 | | | 0.8 |
| 7 | | 0.35 | | | 1.6 |
| 8 | | 0.10 | 0.08 | >1000 | 0.7 |
| 9 | | 0.05 | 0.10 | >1000 | 0.6 |
| 10 | | 0.10 | 0.08 | >1000 | 0.8 |
| 11 | | 0.07 | | | 0.7 |
| 12 | | 0.07 | 0.07 | >1000 | 0.6 |
| 13 | 1.35 | 1.00 | | | NT |
| 14 | | 0.03 | 0.02 | >1000 | 0.8 |
| 15 | | 0.10 | | | 0.5 |
| 16 | | 0.11 | 0.09 | >1000 | 0.6 |
| 17 | | 0.16 | 0.04 | >1000 | 0.8 |
| 18 | | 0.08 | 0.05 | >1000 | 0.6 |
| 19 | | 0.08 | 0.07 | >1000 | 0.6 |
| 20 | | 0.16 | 0.13 | >1000 | 0.8 |
| 21 | | 0.04 | 0.03 | >1000 | 0.9 |
| 22 | | 0.08 | 0.09 | >1000 | 0.6 |
| 23 | | 0.14 | | | 0.8 |
| 24 | | 0.23 | | | 0.7 |
| 25 | | 0.04 | | | 0.8 |
| 26 | | 0.24 | | | 1.5 |
| 27 | | 0.10 | | | 0.5 |
| 28 | | 0.25 | | | 1.6 |
| 29 | 0.94 | 0.65 | 0.28 | >1000 | 3.3 |
| 30 | | 0.07 | 0.05 | >1000 | 0.5 |
| 31 | | 0.04 | | | 0.4 |
| 32 | | 0.12 | | | 0.6 |
| 33 | 0.85 | 1.66 | 0.30 | >1000 | 6.8 |
| 34 | | 0.11 | 0.11 | >1000 | 0.7 |
| 35 | | 0.17 | 0.11 | >1000 | 1.0 |
| 36 | 2.30 | | | | 2.2 |
| 37 | | 0.09 | | | 0.6 |
| 38 | 0.88 | 0.70 | 0.34 | >1000 | 3.1 |
| 39 | 0.33 | 0.17 | 0.05 | >1000 | 0.9 |
| 40 | | 0.37 | | | 1.5 |
| 41 | | 0.07 | 0.12 | >1000 | 0.6 |
| 42 | 1.10 | | | | NT |
| 43 | | 0.09 | | | 0.6 |
| 44 | | 0.14 | 0.16 | >1000 | 0.5 |
| 45 | | 0.05 | 0.01 | >1000 | 0.6 |
| 46 | 0.55 | 0.56 | 0.07 | >1000 | 3.0 |
| 47 | | 0.37 | | | 1.9 |
| 48 | | 0.15 | 0.03 | >1000 | 0.7 |
| 49 | | 0.20 | 0.08 | >1000 | 0.7 |
| 50 | | 0.20 | 0.04 | >1000 | 0.9 |
| 51 | | 0.05 | 0.08 | >1000 | 0.4 |
| 52 | 1.03 | | | | 3.9 |
| 53 | | 0.09 | 0.02 | >1000 | 0.6 |
| 54 | | 0.24 | 0.07 | >1000 | 1.8 |
| 55 | | 0.23 | | | 1.3 |
| 56 | 3.23 | 2.00 | 27.20 | >1000 | 11.0 |
| 57 | | 0.18 | 0.09 | >1000 | 0.8 |
| 58 | 3.34 | 3.14 | | | 16.2 |
| 59 | 0.85 | 1.07 | | | 6.9 |
| 60 | 0.35 | 0.33 | 0.20 | >1000 | 2.3 |
| 61 | 0.73 | 0.65 | | >1000 | 3.6 |
| 62 | 0.75 | 0.64 | | >1000 | 4.4 |
| 63 | | 0.24 | | | 1.2 |
| 64 | | 0.42 | | | 1.1 |
| 65 | 1.50 | | | | 4.0 |
| 66 | 1.33 | | | | NT |
| 67 | | 0.20 | | | 0.4 |
| 68 | 1.03 | | 0.55 | >1000 | 3.0 |
| 69 | | 0.33 | | | 1.6 |
| 70 | | 0.12 | 0.07 | >1000 | 0.9 |
| 71 | 0.45 | 0.27 | | | 2.8 |
| 72 | | 0.09 | 0.05 | >1000 | 0.8 |
| 73 | 0.75 | 0.91 | 0.90 | >1000 | 5.8 |
| 74 | | 0.08 | 0.12 | >1000 | 0.5 |
| 75 | 0.99 | 1.09 | | | 6.1 |
| 76 | 0.65 | | | | 4.7 |
| 77 | | 0.06 | | | 0.6 |
| 78 | | 0.05 | | | 0.7 |
| 79 | | 0.15 | 0.05 | >1000 | 1.3 |
| 80 | | 0.30 | 0.33 | >1000 | 1.2 |
| 81 | | 0.26 | | | 1.1 |
| 82 | | 0.25 | 0.07 | >1000 | 1.1 |
| 83 | | 0.16 | 0.09 | >1000 | 0.6 |
| 84 | | 0.06 | 0.02 | >1000 | 0.6 |
| 85 | | 0.11 | | | 1.1 |
| 86 | | 0.13 | 0.08 | >1000 | 0.5 |
| 87 | | 0.15 | 0.02 | >1000 | 0.7 |
| 88 | | 0.20 | | | 1.9 |
| 89 | | 0.18 | | | 1.2 |
| 90 | | 0.12 | | | 1.2 |
| 91 | | 0.24 | | | 0.9 |
| 92 | | 0.24 | 0.05 | >1000 | 1.1 |
| 93 | 0.65 | 0.55 | 0.78 | >1000 | 2.7 |
| 94 | | 0.18 | 0.07 | >1000 | 0.8 |
| 95 | | 0.14 | 0.20 | >1000 | 0.7 |
| 96 | 0.24 | 0.11 | 0.00 | >1000 | 1.6 |
| 97 | 0.23 | 0.13 | 0.08 | >1000 | 0.9 |
| 98 | | 0.21 | | | 1.5 |
| 99 | 0.55 | 0.56 | 0.15 | >1000 | 3.7 |
| 100 | | 0.28 | | | 1.3 |
| 101 | | 0.05 | 0.05 | >1000 | 0.9 |
| 102 | | 0.16 | 0.20 | >1000 | 0.5 |
| 103 | | 0.05 | | | 0.8 |
| 104 | | 0.09 | 0.02 | >1000 | 1.0 |
| 105 | | 0.12 | | | 0.7 |
| 106 | | 0.05 | 0.04 | >1000 | 0.6 |
| 107 | | 0.30 | | | 1.0 |
| 108 | | 0.27 | | | 1.2 |
| 109 | | 0.11 | 0.05 | >1000 | 0.8 |
| 110 | | 0.14 | 0.14 | | 1.2 |
| 111 | | 0.39 | | | 1.6 |
| 112 | | 0.24 | 0.03 | >1000 | 1.1 |
| 113 | | 0.09 | | | 0.5 |
| 114 | | 0.02 | 0.02 | >1000 | 0.9 |
| 115 | | 0.19 | | | 1.4 |
| 116 | | 0.08 | | | 0.4 |
| 117 | | 0.19 | | | 2.1 |
| 118 | | 0.38 | | | 2.8 |
| 119 | | 0.12 | | | 0.7 |
| 120 | | 0.38 | | | 1.4 |
| 121 | | 0.07 | 0.02 | >1000 | 0.9 |
| 122 | | 0.08 | 0.04 | >1000 | 0.9 |
| 123 | 0.65 | 1.36 | 0.32 | >1000 | 4.1 |
| 124 | 0.30 | 0.59 | 0.12 | >1000 | 1.5 |
| 125 | 1.14 | | | | 4.1 |
| 126 | | 0.08 | | | 0.8 |
| 127 | | 0.22 | 0.18 | >1000 | 0.8 |
| 128 | | 0.09 | 0.02 | >1000 | 0.8 |
| 129 | | 0.48 | | | 3.1 |
| 130 | | 0.01 | 0.06 | >1000 | 0.7 |
| 131 | | 0.15 | 0.06 | >1000 | 0.7 |
| 132 | | 0.13 | 0.08 | >1000 | 0.7 |
| 133 | | 0.13 | | | 1.4 |
| 134 | | 0.06 | 0.05 | >1000 | 0.4 |
| 135 | | 0.07 | | | 0.4 |
| 136 | | 0.07 | | | 0.4 |
| 137 | | 0.21 | | | 1.3 |
| 138 | | 0.38 | | | 2.0 |
| 139 | | 0.13 | | | 1.0 |
| 140 | | 0.06 | 0.05 | >1000 | 0.5 |
| 141 | | 0.17 | 0.06 | >1000 | 1.2 |
| 142 | | 0.33 | | | 1.1 |
| 143 | 0.40 | 0.21 | 0.05 | >1000 | 2.2 |
| 144 | | 0.07 | 0.10 | >1000 | 0.6 |
| 145 | | 0.18 | | | 0.8 |
| 146 | 0.59 | | | | 5.4 |
| 147 | | 0.21 | | | 1.4 |
| 148 | | 0.90 | 1.10 | >1000 | 2.8 |
| 149 | | 0.21 | 0.18 | >1000 | 1.3 |
| 150 | | 0.23 | | | 1.1 |
| 151 | 0.29 | 0.11 | 0.04 | >1000 | 1.1 |
| 152 | 0.99 | 1.19 | | | 5.3 |
| 153 | | 0.23 | 0.16 | >1000 | 2.6 |
| 154 | | 0.17 | | | 1.2 |
| 155 | | 0.10 | 0.02 | >1000 | 0.9 |
| 156 | | 0.12 | | | 0.8 |
| 157 | 0.72 | 0.64 | 0.50 | >1000 | 4.0 |
| 158 | 1.79 | | | | 5.3 |
| 159 | | 0.12 | 0.05 | >1000 | 0.8 |
| 160 | | 0.23 | | | 1.5 |
| 161 | | 0.09 | 0.02 | >1000 | 1.3 |
| 162 | 0.69 | 0.56 | | | 0.1 |
| 163 | | 0.05 | 0.05 | >1000 | 0.7 |
| 164 | 0.60 | 0.50 | | | 3.6 |
| 165 | | 0.18 | | | 1.1 |
| 166 | 0.50 | 0.44 | | | 2.7 |
| 167 | | 0.48 | | | 2.6 |
| 168 | 0.30 | | 0.37 | >1000 | 1.2 |
| 169 | | 0.16 | 0.08 | >1000 | 0.6 |
| 170 | 0.60 | 0.56 | | | NT |
| 171 | | 0.19 | | | 0.9 |
| 172 | 0.63 | 0.57 | 0.20 | >1000 | 3.9 |
| 173 | | 0.26 | | | 1.1 |
| 174 | | 0.06 | 0.01 | >1000 | 0.7 |
| 175 | | 0.22 | | | 0.9 |
| 176 | | 0.22 | | | 1.0 |
| 177 | 0.75 | 0.54 | | | 3.3 |
| 178 | | 0.29 | | | 3.3 |
| 179 | 0.50 | 0.29 | 0.14 | >1000 | 2.4 |
| 180 | 0.30 | 0.18 | 0.05 | >1000 | 2.0 |
| 181 | | 0.34 | | | 1.2 |
| 182 | | 0.20 | | | 0.9 |
| 183 | 0.50 | 0.17 | 0.10 | >1000 | 2.8 |
| 184 | 0.50 | | | | 1.7 |
| 185 | 0.20 | 0.16 | | | 0.9 |
| 186 | 0.50 | 0.42 | | | 1.9 |
| 187 | | 0.09 | 0.04 | >1000 | 0.5 |
| 188 | | 0.10 | 0.03 | >1000 | 0.8 |
| 189 | | 0.01 | 0.10 | >1000 | 0.6 |
| 190 | | 0.33 | | | 1.6 |
| 191 | | 0.18 | | | 1.6 |
| 192 | | 0.24 | | | 1.1 |
| 193 | | 0.15 | | | 1.0 |
| 194 | | 0.05 | 0.04 | >1000 | 1.1 |
| 195 | | 0.28 | | | 1.6 |
| 196 | | 0.09 | 0.05 | >1000 | 0.6 |
| 197 | | 0.36 | | | 0.7 |
| 198 | | 0.31 | | | 0.8 |
| 199 | | 0.32 | | | 1.0 |
| 200 | | 0.09 | 0.02 | >1000 | 1.0 |
| 201 | | 0.21 | | | 0.9 |
| 202 | | 0.23 | | | 1.4 |
| 203 | 0.45 | 0.31 | 0.10 | >1000 | 1.6 |
| 204 | | 0.11 | | | 0.8 |
| 205 | 0.99 | 1.01 | 0.60 | >1000 | 7.2 |
| 206 | 26.0 | | 3.00 | >1000 | 270.6 |
| 207 | | 0.12 | | | 1.0 |
| 208 | 1.31 | | | | 4.0 |
| 209 | 0.60 | 0.67 | 0.40 | >1000 | 2.7 |
| 210 | | 0.12 | | | 0.5 |
| 211 | 0.79 | 0.63 | | | 3.3 |
| 212 | 0.90 | 0.73 | 0.50 | >1000 | 2.8 |
| 213 | | 0.31 | | | 1.0 |
| 214 | 0.95 | 0.61 | | | 3.7 |
| 215 | 2.85 | | 1.80 | >1000 | 9.7 |
| 216 | 0.65 | | | | NT |
| 217 | 1.00 | 0.73 | | | 4.4 |
| 218 | 1.15 | | 0.50 | >1000 | 3.4 |
| 219 | 0.30 | 0.15 | 0.10 | >1000 | 1.6 |
| 220 | 0.75 | 0.55 | 0.25 | >1000 | 2.9 |
| 221 | | 0.31 | 0.14 | >1000 | 1.0 |
| 222 | 1.73 | | 0.18 | >1000 | 5.7 |
| 223 | 0.39 | 0.48 | 0.30 | >1000 | 2.9 |
| 224 | 1.70 | | 0.40 | >1000 | 4.8 |
| 225 | | 0.27 | | | 0.9 |
| 226 | 1.16 | | | | 10.0 |
| 227 | 4.02 | | 2.30 | >1000 | 13.5 |
| 228 | 0.70 | 0.64 | | | 4.0 |
| 229 | | 0.10 | 0.14 | >1000 | 0.5 |
| 230 | 0.59 | 0.43 | 0.21 | >1000 | 2.7 |
| 231 | 2.63 | | 1.90 | >1000 | 6.1 |
| 232 | 3.19 | 2.15 | 0.70 | >1000 | 6.3 |
| 233 | 2.65 | | 0.78 | >1000 | 14.2 |
| 234 | | 0.24 | | | 1.1 |
| 235 | 1.92 | | 1.20 | >1000 | 5.0 |
| 236 | 0.90 | | | | 4.2 |
| 237 | 13.04 | | | | 20.8 |
| 238 | | 0.12 | | | 0.7 |
| 239 | 0.52 | 0.61 | 0.30 | >1000 | 1.4 |
| 240 | | 0.23 | 0.09 | >1000 | 1.0 |
| 241 | | 0.24 | | | 1.4 |
| 242 | 1.21 | | 0.30 | >1000 | 3.0 |
| 243 | 0.55 | 0.49 | 0.06 | >1000 | 3.2 |
| 244 | 0.49 | 0.96 | 0.09 | >1000 | 4.5 |
| 245 | 0.95 | | | | 9.4 |
| 246 | 1.15 | | | | 4.3 |
| 247 | | 0.09 | 0.07 | >1000 | 0.5 |
| 248 | 1.45 | 1.56 | 2.00 | >1000 | 10.6 |
| 249 | 1.00 | | | | 4.4 |
| 250 | 0.27 | 0.17 | | | 2.4 |
| 251 | 0.50 | 1.17 | | | 2.7 |
| 252 | 2.39 | | | | 23.3 |
| 253 | 0.77 | 1.02 | 1.09 | >1000 | 4.4 |
| 254 | | 0.06 | 0.02 | >1000 | 0.4 |
| 255 | 0.70 | 1.01 | | | 6.5 |
| 256 | 1.84 | | 1.40 | >1000 | 6.1 |
| 257 | | 0.08 | | | 0.4 |
| 258 | 1.00 | 1.03 | 0.20 | >1000 | 11.5 |
| 259 | 0.35 | 0.22 | 0.15 | >1000 | 1.9 |
| 260 | 10.15 | | 6.00 | >1000 | 50.1 |
| 261 | 0.40 | 0.30 | 0.20 | >1000 | 2.7 |
| 262 | | 0.39 | 0.24 | >1000 | 1.2 |
| 263 | 2.88 | | 1.60 | >1000 | 4.7 |
| 264 | 1.49 | | | | 8.1 |
| 265 | 1.1 | | 1.00 | >1000 | 4.9 |
| 266 | | 0.45 | 0.06 | >1000 | 0.9 |
| 267 | 0.24 | 0.29 | 0.14 | >1000 | 1.7 |
| 268 | 0.77 | 0.45 | 0.27 | >1000 | 2.0 |
| 269 | | 0.08 | 0.10 | >1000 | 1.0 |
| 270 | 0.55 | | | | 2.2 |
| 271 | 0.94 | 0.95 | 0.28 | >1000 | 2.7 |
| 272 | 0.30 | 0.21 | 0.10 | >1000 | 2.6 |
| 273 | 0.60 | 0.33 | | | 4.1 |
| 274 | 0.86 | | | | 3.2 |
| 275 | | 0.36 | 0.30 | >1000 | 1.4 |
| 276 | 2.41 | | | | NT |
| 277 | 1.12 | | | | 3.0 |
| 278 | 8.69 | | 12.75 | >1000 | 54.5 |
| 279 | | 0.38 | | | 1.4 |
| 280 | | 0.25 | | | 1.1 |
| 281 | 1.40 | | 0.50 | >1000 | 8.4 |
| 282 | | 0.13 | | | 0.9 |
| 283 | 1.45 | | 0.50 | >1000 | 6.4 |
| 284 | | 0.09 | | | 0.8 |
| 285 | | 0.04 | | | 0.6 |
| 286 | | 0.05 | | | 0.4 |
| 287 | | 0.22 | | | 1.7 |
| 288 | 0.20 | 0.11 | 0.10 | >1000 | 4.2 |
| 289 | 9.87 | | 7.43 | >1000 | 36.4 |

What is claimed is:

1. A compound of Formula (I) or a pharmaceutically acceptable salt thereof, wherein 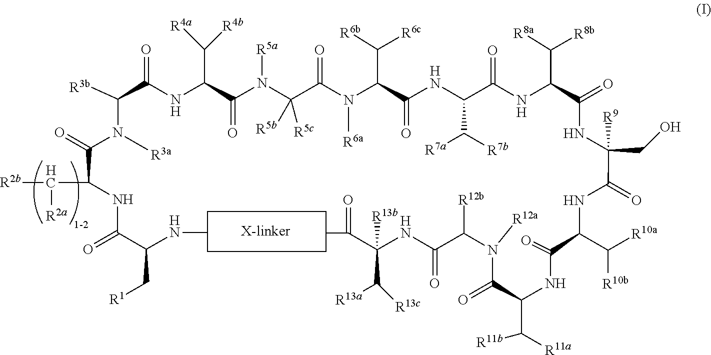
R¹ is selected from hydrogen, C₁₋₁₀ alkyl, (C₁₋₆ alkyl)₀₋₂ amino(C₀₋₁₀ alkyl), (C₁₋₆ alkyl)₀₋₂ amino(C₀₋₁₀ alkyl)oxy(C₀₋₆ alkyl), (C₁₋₆ alkyl)₃N⁺(C₀₋₆ alkyl), aryl(C₀₋₁₀ alkyl), heteroaryl(C₀₋₁₀ alkyl), (C₃₋₁₂)cycloalkyl(C₀₋₁₀ alkyl), heterocycloalkyl(C₀₋₁₀ alkyl), C₁₋₁₀ fluoroalkyl, C₂₋₁₀ alkenyl, (C₀₋₆ alkyl)carbonylamino(C₀₋₆ alkyl), (C₀₋₆ alkyl) 0-2 aminocarbonyl(C₀₋₆ alkyl), (C₁₋₆ alkyl)₀₋₂ aminocarbonylamino(C₀₋₆ alkyl), arylcarbonylamino(C₀₋₆ alkyl), arylaminocarbonyl(C₀₋₆ alkyl), heteroarylcarbonylamino(C₀₋₆ alkyl), heteroarylaminocarbonyl(C₀₋₆ alkyl), C₁₋₆ alkyloxy, (C₁₋₆ alkyl)oxy(C₀₋₆ alkyl), ((C₃₋₁₂)cycloalkyl)oxy(C₀₋₆ alkyl), ((C₃₋₁₂)cycloalkyl C₀₋₆ alkyl)oxy(C₀₋₆ alkyl), (C₀₋₆ alkyl)carboxy(C₀₋₆ alkyl), N⁻═N⁺═N—(C₀₋₆ alkyl), and H₂N—C(═NH)NH—(C₀₋₆ alkyl),
wherein R¹ is substituted by 0, 1, 2, 3, or 4 Ria substituents each independently selected from C₁₋₆ alkyl, amino, cyano, halo, hydroxy, (C₃₋₁₂)cycloalkyloxy, and C₁₋₆ alkyloxy;
each R^{2a} is independently selected from hydrogen, hydroxy, C₁₋₄ alkyl, fluoro, and C₁₋₄ alkyloxy;
R^{2b} is selected from aryl, heteroaryl, cycloalkyl, and heterocycloalkyl, wherein said heteroaryl contains at least one nitrogen atom,
wherein R^{2b} is substituted by 0, 1, or 2 R^{2c} independently selected from C₁₋₆ alkyl, amino(C₀₋₆ alkyl), (C₁₋₆ alkyl)₀₋₂ amino(C₀₋₆ alkyl), (C₁₋₆ alkyl)₃N⁺(C₀₋₆ alkyl), aminocarbonyl(C₀₋₆ alkyl), (C₁₋₆ alkyl)₀₋₂ aminocarbonyl(C₀₋₆ alkyl), hydroxy, C₁₋₆ alkyloxy, halo, (C₁₋₆ alkyl)₀₋₂ amino(C₀₋₆ alkyloxy), (C₁₋₆ alkyl)₃N⁺(C₀₋₆ alkyloxy), (C₁₋₆ alkyloxy)carbonyl(C₀₋₆ alkyl), carboxy(C₀₋₆ alkyl), carboxy(C₁₋₆ alkyl)oxy(C₀₋₆ alkyl), C₁₋₆ haloalkyl, C₁₋₆ haloalkyloxy, and C₁₋₆ alkyloxy;
R^{3a} is selected from hydrogen, hydroxy, hydroxy(C₁₋₆ alkyl), amino, amino(C₁₋₆ alkyl), C₁₋₁₀ alkyl, (C₃₋₁₂)cycloalkyl(C₀₋₁₀ alkyl), (C₀₋₆ alkyl)thio(C₁₋₆ alkyl), and carboxy(C₁₋₆ alkyl), wherein R^{3a} may be substituted by 0, 1, or 2 R^{3c} substituents;
R^{3b} is selected from hydrogen, C₁₋₁₀ alkyl, hydroxy(C₁₋₆ alkyl), amino(C₁₋₁₀ alkyl), (C₁₋₆ alkyl)₀₋₂ amino(C₁₋₁₀ alkyl), (C₁₋₆ alkyl)₃N⁺(C₁₋₆ alkyl), C₁₋₆ haloalkyl, aryl(C₀₋₁₀ alkyl), heteroaryl(C₀₋₁₀ alkyl), (C₃₋₁₂)cycloalkyl(C₀₋₁₀ alkyl), heterocycloalkyl(C₀₋₁₀ alkyl), (C₁₋₆ alkyl)oxy(C₁₋₆ alkyl), (C₃₋₁₂)cycloalkyloxy(C₁₋₆ alkyl), carboxy(C₁₋₆ alkyl), aminocarbonyl(C₁₋₆ alkyl), (C₁₋₆ alkyl)₀₋₂ aminocarbonyl(C₁₋₆ alkyl), aminocarbonylamino(C₁₋₆ alkyl), (C₁₋₆ alkyl)₀₋₂ aminocarbonylamino(C₁₋₆ alkyl), (C₀₋₆ alkyl)thio(C₁₋₆ alkyl), (C₁₋₆ alkyl)SO₂(C₁₋₆ alkyl), and (C₁₋₆ alkyl)sulfinyl(C₁₋₆ alkyl), wherein R^{3b} may be substituted by 0, 1, or 2 R^{3c} substituents;
each R^{3c} is independently selected from halo, C₁₋₆ alkyl, amino, (C₁₋₆ alkyl)₀₋₂ amino(C₀₋₆ alkyl), (C₁₋₆ alkyl)₃N⁺—, (C₁₋₆ alkyl)SO₂(C₀₋₆ alkyl), cyano, cyano(C₁₋₆ alkyl), hydroxy, hydroxy(C₁₋₆ alkyl), (C₁₋₆ alkyl)oxy(C₀₋₆ alkyl), aminocarbonyl(C₀₋₆ alkyl), and (C₀₋₆) carboxy(C₀₋₆ alkyl);
and wherein R^{3a} and R^{3b}, together with the atoms to which they are attached, may form a saturated ring system, wherein said saturated ring system may be substituted by 0, 1, or 2 R^{3c} substituents;
R^{4a} is selected from hydrogen, C₁₋₆ alkyl, hydroxy, C₁₋₄ alkyloxy, and fluoro;
R^{4b} is selected from bicyclic heteroaryl(C₀₋₃ alkyl), and bicyclic aryl(C₀₋₃ alkyl), wherein R^{4b} is substituted with 0, 1, or 2 R^{4c} substituents each independently selected from halo, hydroxy, cyano, nitro, carboxy, carboxy(C₁₋₆ alkyl), (C₁₋₆ alkyloxy)carbonyl(C₀₋₆ alkyl), C₁₋₆ alkyloxy, (C₁₋₆ alkyl)oxy(C₁₋₆ alkyl), C₁₋₆ alkyl, and C₁₋₆ haloalkyl;
R^{5a} is selected from hydrogen, C₁₋₁₀ alkyl, C₁₋₁₀ fluoroalkyl, carboxy(C₁₋₁₀ alkyl), hydroxy, hydroxy(C₁₋₁₀ alkyl), cyano(C₁₋₁₀ alkyl), heterocycloalkyl(C₀₋₁₀ alkyl), aminocarbonyl(C₁₋₆ alkyl), (C₁₋₆ alkyl)₀₋₂ aminocarbonyl(C₁₋₆ alkyl), (carboxy(C₁₋₁₀ alkyl))oxy(C₁₋₆ alkyl), aryl(C₀₋₆ alkyl), (C₃₋₁₂)cycloalkyl(C₀₋₆ alkyl), heteroaryl(C₀₋₆ alkyl), (C₁₋₆ alkyl)oxy(C₁₋₆ alkyl), (C₁₋₆ alkyloxy)carbonyl(C₀₋₆ alkyl), amino, amino(C₁₋₆ alkyl), (C₁₋₆ alkyl)₀₋₂ amino(C₀₋₆ alkyl), amino(C₁₋₆ alkyl)carbonylamino(C₁₋₆ alkyl), (C₁₋₆ alkyl)₀₋₂ amino(C₁₋₆ alkyl)carbonylamino(C₁₋₆ alkyl), (C₁₋₆ alkyl)₃N⁺(C₂₋₁₀ alkyl), (C₁₋₆ alkyl)₃N⁺(C₁₋₆ alkyl)carbonylamino(C₁₋₆ alkyl), and C₁₋₁₀ fluoroalkyl, and wherein R^{5a} is substituted by 0, 1, or 2 R^{5d} substituents;
R^{5b} is selected from hydrogen, C₁₋₁₀ alkyl, hydroxy(C₁₋₁₀ alkyl), carboxy(C₁₋₆ alkyl), (C₁₋₆ alkyl)oxy(C₁₋₆ alkyl), aminocarbonyl(C₁₋₆ alkyl), (C₁₋₆ alkyl)₀₋₂ aminocarbonyl(C₁₋₆ alkyl), carboxy(C₁₋₁₀ alkyl)oxy(C₁₋₆ alkyl), cyano(C₁₋₁₀ alkyl), amino(C₁₋₆ alkyl), (C₁₋₆ alkyl)₀₋₂ amino(C₁₋₆ alkyl), amino(C₁₋₆ alkyl)carbonylamino(C₁₋₆ alkyl), (C₁₋₆ alkyl)₀₋₂ amino(C₁₋₆ alkyl)carbonylamino(C₁₋₆ alkyl), (C₁₋₆ alkyl)₃N⁺(C₂₋₆ alkyl)oxy(C₁₋₆ alkyl), (C₁₋₆ alkyl)₃N⁺(C₁₋₆ alkyl)carbonylamino(C₁₋₆ alkyl), (C₁₋₆ alkyl)₃N⁺(C₁₋₆ alkyl), heterocycloalkyl(C₀₋₁₀ alkyl), (C₃₋₁₂)cycloalkyl(C₀₋₁₀ alkyl), and C₁₋₁₀ haloalkyl, wherein R^{5b} is substituted by 0, 1, 2, or 3 R^{5e} substituents,
wherein R^{5a} and R^{5b}, together with the atoms to which they are attached, form a saturated mono- or bi-cyclic ring, wherein said mono- or bi-cyclic ring is substituted with 0, 1, 2, or 3 R^{5d} and 0, 1, 2, or 3 R^{5e};
each R^{5d} is independently selected from selected from halo, hydroxy, hydroxy(C₁₋₁₀ alkyl), C₁₋₁₀ alkyl, carboxy, carboxy(C₁₋₆ alkyl), C₁₋₆ alkyloxy, (C₁₋₆ alkyl)oxy(C₁₋₆ alkyl), amino, amino(C₁₋₆ alkyl), (C₁₋₆ alkyl)₀₋₂ amino(C₀₋₆ alkyl), amino(C₁₋₆ alkyl)carbonylamino (C₀₋₆ alkyl), (C₁₋₆ alkyl)₀₋₂ amino(C₁₋₆ alkyl)carbonylamino(C₀₋₆ alkyl), (C₁₋₆ alkyl)₃N⁺(C₀₋₆ alkyl), (C₁₋₆ alkyl)₃N⁺(C₂₋₆ alkyl)oxy(C₀₋₆ alkyl), (C₁₋₆ alkyl)₃N⁺(C₁₋₆ alkyl)carbonylamino (C₀₋₆ alkyl), (carboxy(C₁₋₆ alkyl))oxy(C₁₋₆ alkyl), cyano(C₀₋₆ alkyl), tetrazolyl(C₀₋₆ alkyl), and C₁₋₆ haloalkyl, and two R^{5d} substituents together with the atom they are attached to may join together to form a saturated ring;
each R^{5e} is independently selected from halo, hydroxy, hydroxy(C₁₋₁₀ alkyl), C₁₋₁₀ alkyl, carboxy, carboxy(C₁₋₆ alkyl), C₁₋₆ alkyloxy, (C₁₋₆ alkyl)oxy(C₁₋₆ alkyl), amino, amino(C₁₋₆ alkyl), (C₁₋₆ alkyl)₀₋₂ amino(C₀₋₆ alkyl), amino(C₁₋₆ alkyl)carbonylamino(C₀₋₆ alkyl), (C₁₋₆ alkyl)₀₋₂ amino(C₁₋₆ alkyl)carbonylamino(C₀₋₆ alkyl), (C₁₋₆ alkyl)₃N⁺(C₀₋₆ alkyl), (C₁₋₆ alkyl)₃N⁺(C₂₋₆ alkyl)oxy(C₀₋₆ alkyl), (C₁₋₆ alkyl)₃N⁺(C₁₋₆ alkyl)carbonylamino (C₀₋₆ alkyl), (carboxy(C₁₋₆ alkyl))oxy(C₁₋₆ alkyl), cyano(C₀₋₆ alkyl), tetrazolyl(C₀₋₆ alkyl), and C₁₋₆ haloalkyl, and two R^{5e} substituents together with the atom they are attached to may join together to form a saturated ring;
R^{5c} is hydrogen, C₁₋₄ alkyl, hydroxy(C₁₋₄ alkyl), C₁₋₄ alkyloxy, or (C₁₋₄ alkyl)oxy (C₁₋₄ alkyl);
R^{6a} is selected from hydrogen, hydroxy, amino, C₁₋₄ alkyloxy, and C₁₋₆ alkyl;
R^{6b} is selected from hydrogen, C₁₋₆ alkyl, hydroxy, C₁₋₄ alkyloxy, and fluoro;
R^{6c} is selected from C₁₋₁₀ alkyl, C₁₋₆ alkyloxy, (C₁₋₆ alkyl)oxy(C₁₋₆ alkyl), C₁₋₁₀ haloalkyl, aryl(C₀₋₆ alkyl), heteroaryl(C₀₋₁₀ alkyl), (C₁₋₆ alkyl)SO₂(C₀₋₆ alkyl), hydroxy, hydroxy(C₁₋₆ alkyl), amino(C₀₋₆ alkyl), (C₁₋₆ alkyl)₀₋₂ amino(C₀₋₆ alkyl), aminocarbonyl(C₀₋₆ alkyl), (C₁₋₆ alkyl)₀₋₂ aminocarbonyl(C₀₋₆ alkyl), (C₁₋₆ alkyl) carbonylamino(C₀₋₆ alkyl), (C₁₋₆ alkyl)₀₋₂ aminocarbonylamino(C₀₋₆ alkyl), and carboxy(C₀₋₆ alkyl);
R^{7a} is selected from hydrogen, C₁₋₆ alkyl, hydroxy, C₁₋₄ alkyloxy, and fluoro;
R^{7b} is selected from aryl(C₀₋₆ alkyl), heteroaryl(C₀₋₆ alkyl), and (C₃₋₁₂)cycloalkyl(C₀₋₆ alkyl),
wherein R^{7b} is substituted by 0, 1, 2, or 3 R^{7c} substituents each independently selected from C₁₋₆ alkyl, C₁₋₁₀ fluoroalkyl, C₁₋₁₀ fluoroalkyloxy, (C₁₋₆ alkyl)₀₋₂ amino(C₀₋₅ alkyl), (C₁₋₆ alkyl)₃N⁺(C₀₋₅ alkyl), carboxy(C₀₋₆ alkyl), (C₁₋₆ alkyloxy)carbonyl(C₀₋₆ alkyl), carboxy(C₁₋₆ alkyl)oxy(C₀₋₆ alkyl), aminocarbonyl(C₀₋₆ alkyl), (C₁₋₆ alkyl)₀₋₂ aminocarbonyl (C₀₋₆ alkyl), hydroxy, hydroxy(C₁₋₆ alkyl), halo, —(C₀₋₅ alkyl)-(S(═O)₂OH), —(C₀₋₅ alkyl)-(S(═O)₂NH₂), amino(C₀₋₆ alkyl)oxy(C₀₋₆ alkyl), (C₁₋₆ alkyl)₀₋₂ amino(C₀₋₆ alkyl)oxy(C₀₋₆ alkyl), (C₁₋₆ alkyl)₃N⁺(C₀₋₆ alkyl)oxy(C₀₋₆ alkyl), aminocarbonylamino(C₀₋₆ alkyl), (C₁₋₆ alkyl)₀₋₂ aminocarbonylamino(C₀₋₆ alkyl), C₁₋₁₀ haloalkyl, C₁₋₁₀ haloalkyloxy, and (C₁₋₆ alkyl)oxy(C₀₋₆ alkyl);
R^{8a} is selected from hydrogen, C₁₋₆ alkyl, hydroxy, C₁₋₄ alkyloxy, C₁₋₆ fluoroalkyl, C₁₋₄ fluoroalkyloxy, and halo;
R^{8b} is selected from bicyclic aryl(C₀₋₃ alkyl) and bicyclic heteroaryl(C₀₋₃ alkyl),
wherein R^{8b} is substituted by 0, 1, or 2 R^{8c} substituents each independently selected from C₁₋₄ alkyl, halo, cyano, nitro, carboxy, amino, hydroxy, C₁₋₆ fluoroalkyl, C₁₋₆ fluoroalkyloxy, C₁₋₆ alkyloxy, amino(C₁₋₃ alkyl), and hydroxy(C₁₋₆ alkyl);
R⁹ is selected from hydrogen, and C₁₋₄ alkyl;
R^{10a} is selected from hydrogen, hydroxy, C₁₋₆ alkyl, C₁₋₄ alkyloxy, and fluoro;
R^{10b} is selected from (C₃₋₁₂)cycloalkyl(C₀₋₃ alkyl), aryl(C₀₋₃ alkyl), and heteroaryl(C₀₋₃ alkyl), wherein said heteroaryl contains 1, 2, or 3 nitrogen atoms,
wherein R^{10b} is substituted by 0, 1, or 2 R^{10c} wherein each R^{10c} is independently selected from C₁₋₁₀ alkyl, C₁₋₁₀ fluoroalkyl, C₁₋₆ haloalkyl, C₁₋₁₀ fluoroalkyloxy, amino, amino(C₁₋₆ alkyl), (C₁₋₆ alkyl)₀₋₂ amino(C₀₋₆ alkyl), (C₁₋₆ alkyl)₃N⁺(C₀₋₆ alkyl), hydroxy, hydroxy(C₁₋₆ alkyl), cyano, halo, aminocarbonyl, aminocarbonyl(C₁₋₆ alkyl), (C₁₋₆ alkyl)₀₋₂ aminocarbonyl(C₀₋₆ alkyl), (C₁₋₆ alkyl)₀₋₂ aminocarbonylamino(C₀₋₆ alkyl), (C₁₋₆ alkyl) carbonylamino(C₀₋₆ alkyl), carboxy(C₀₋₆ alkyl), (C₁₋₆ alkoxy)carbonyl(C₀₋₆ alkyl), carboxy(C₁₋₆ alkyl)oxy(C₀₋₆ alkyl), —(C₀₋₅ alkyl)-(S(═O)₂OH), —(C₀₋₅ alkyl)-(S(═O)₂NH₂), amino(C₀₋₆ alkyl)oxy(C₀₋₆ alkyl), (C₁₋₆ alkyl)₀₋₂ amino(C₀₋₆ alkyl)oxy(C₀₋₆ alkyl), (C₁₋₆ alkyl)₃N⁺(C₀₋₆ alkyl)oxy(C₀₋₆ alkyl), (C₁₋₆ alkyl)oxy(C₀₋₆ alkyl), C₁₋₆ haloalkyloxy, (C₃₋₁₂) cycloalkyl(C₀₋₆ alkyl), and heterocycloalkyl(C₀₋₆ alkyl);
R^{11a} is selected from hydrogen, hydroxy, C₁₋₆ alkyl, C₁₋₄ alkyloxy, and fluoro;
R^{11b} is selected from aryl(C₀₋₃ alkyl), heteroaryl(C₀₋₃ alkyl), wherein said heteroaryl contains 1, 2, or 3 nitrogen atoms, and H₂N—C(═NH)NH—(C₁₋₆ alkyl),
wherein R^{11b} is substituted by 0, 1, 2, 3, or 4 R^{11c} substituents each independently selected from C₁₋₆ alkyl, amino, amino(C₁₋₆ alkyl), (C₁₋₆ alkyl)₀₋₂ amino(C₀₋₆ alkyl), (C₁₋₆ alkyl)₃N⁺(C₀₋₆ alkyl), hydroxy, hydroxy(C₁₋₆ alkyl), cyano, halo, aminocarbonyl, aminocarbonyl(C₁₋₆ alkyl), (C₁₋₆ alkyl)₀₋₂ aminocarbonyl(C₀₋₆ alkyl), carboxy(C₀₋₆ alkyl), carboxy(C₁₋₆ alkyl)oxy(C₀₋₆ alkyl), amino(C₀₋₆ alkyl)oxy(C₀₋₆ alkyl), (C₁₋₆ alkyl)₀₋₂ amino (C₀₋₆ alkyl)oxy(C₀₋₆ alkyl), (C₁₋₆ alkyl)₃N⁺(C₀₋₆ alkyl)oxy(C₀₋₆ alkyl), (C₁₋₆ alkyl)oxy(C₀₋₆ alkyl), C₁₋₆ haloalkyloxy, C₁₋₆ haloalkyl, (C₃₋₁₂)cycloalkyl(C₀₋₆ alkyl), heterocycloalkyl(C₀₋₆ alkyl), ((C₁₋₆ alkyl)carbonyl)heterocycloalkyl(C₀₋₁₀ alkyl), and ((C₁₋₆ alkyl)carbonyloxy) heterocycloalkyl(C₀₋₁₀ alkyl);
R^{12a} is selected from hydrogen, hydroxy, amino, C₁₋₁₀ alkyl, and (C₃₋₁₂)cycloalkyl (C₀₋₆ alkyl);
R^{12b} is selected from hydrogen, C₁₋₁₀ alkyl, and (C₃₋₁₂)cycloalkyl(C₀₋₆ alkyl);
wherein R^{12a} and R^{12b}, together with the atoms to which they are attached, may form a saturated ring;
R^{13a} is selected from hydrogen, hydroxy, C₁₋₆ alkyl, C₁₋₄ alkyloxy, and fluoro;
R^{13b} is selected from hydrogen, and C₁₋₄ alkyl;
R^{13c} is selected from a bicyclic nitrogen-containing heteroaryl having 1 or 2 nitrogen and bicyclic-aryl and wherein R^{13c} is substituted independently by 0, 1, or 2 R^{13d} substituents each independently selected from halo, C₁₋₆ alkyl, carboxy(C₀₋₄ alkyl), C₁₋₄ haloalkyloxy, and C₁₋₄ alkyloxy; 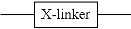
is selected from 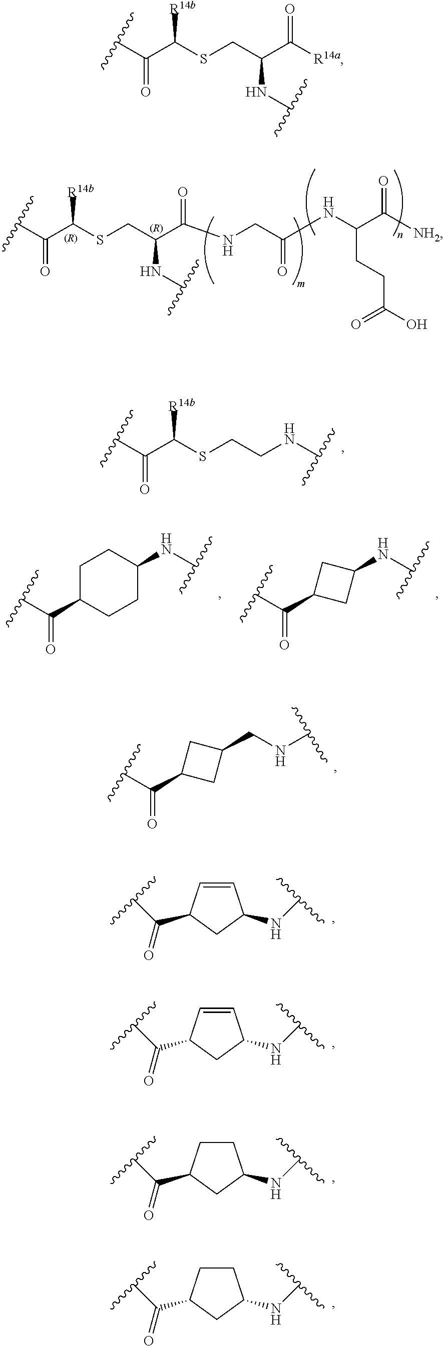 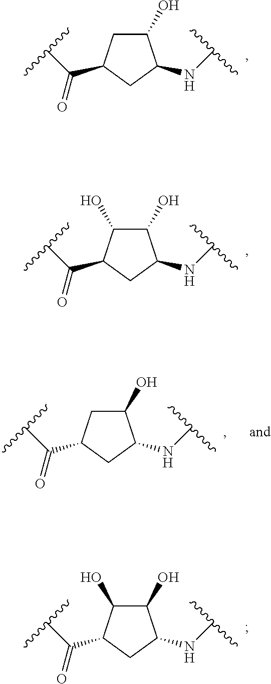
wherein R^{14a} is selected from amino, hydroxy, (C₁₋₆ alkyl)₀₋₂ amino, and C₁₋₆ alkyloxy;
m is selected from 1, 2, 3, or 4;
n is selected from 1, 2, 3, or 4; and
R^{14b} is selected from hydrogen, C₁₋₈ alkyl, aryl(C₀₋₆ alkyl), and heteroaryl(C₀₋₆ alkyl), wherein R^{14b} is substituted by 0, 1, 2, or 3 halo groups.

2. The compound of claim 1 or a pharmaceutically acceptable salt thereof, wherein R 1 is selected from aminomethyl, aminoethyl, aminopropyl, aminobutyl, aminopentyl, phenyl, phenylmethyl, phenylethyl, phenylpropyl, styryl, biphenyl, naphthyl, pyridyl, pyridazinyl, pyrimidyl, pyrazinyl, imidazolyl, pyrazolyl, furyl, thiophenyl, oxazolyl, isoxazolyl, thiazolyl, isothiazolyl, oxadiazolyl, biphenylmethyl, naphthylmethyl, pyridylmethyl, pyridazinylmethyl, pyrimidylmethyl, pyrazinylmethyl, imidazolylmethyl, pyrazolylmethyl, furylmethyl, thiophenylmethyl, oxazolylmethyl, isoxazolylmethyl, thiazolylmethyl, isothiazolylmethyl, oxadiazolylmethyl, bicyclo[1.1.1.]pentyl, (bicyclo[1.1.1.]pentyl)methyl, phenylcarbonylaminoethyl, aminocarbonylmethyl, aminocarbonylethyl, aminocarbonylpropyl, aminocarbonylisopropyl, aminocarbonylbutyl, aminocarbonylaminomethyl, aminocarbonylaminoethyl, aminocarbonylaminopropyl, aminocarbonylaminobutyl, methyl, ethyl, propyl, isopropyl, butyl, sec-butyl, isobutyl, tert-butyl, n-pentyl, isopentyl, neopentyl, n-hexyl, isohexyl, n-heptyl, n-octyl, cyclopropyl, cyclobutyl, cyclopentyl, cyclohexyl, cyclopropylmethyl, cyclobutylmethyl, cyclopentylmethyl, cyclohexylmethyl, adamantyl, carboxy, carboxymethyl, carboxyethyl, azidomethyl, azidoethyl, azidopropyl, phenylaminocarbonylmethyl, pyridylaminocarbonylmethyl, (pyridylcarbonylamino)methyl, guanidino, guanidinomethyl, guanidinoethyl, guanidinopropyl, guanidinobutyl, fluoromethyl, difluoromethyl, trifluoromethyl, fluoroethyl, difluoroethyl, trifluoroethyl, pentafluoroethyl, fluoropropyl, difluoropropyl, trifluoropropyl, pentafluoropropyl, heptafluoropropyl, trifluorobutyl, N,N,N-trimethylmethylammonium, N,N,N-trimethyleth-1-ylammonium, N,N,N-trimethylpropan-1-ylammonium, N,N,N-trimethylbut-1-ylammonium, methylamino, methylaminomethyl, methylaminoethyl, methylaminopropyl, methylaminobutyl, dimethylamino, dimethylaminomethyl, dimethylaminoethyl, dimethylaminopropyl, dimethylaminobutyl, ethylamino, ethylaminomethyl, ethylaminoethyl, ethylaminopropyl, ethylaminobutyl, diethylamino, diethylaminomethyl, diethylaminoethyl, diethylaminopropyl, diethylaminobutyl, aminoethoxy, aminoethoxymethyl, isoxazolylcarbonylaminomethyl, methoxy, ethoxy, propoxy, isopropoxy, cyclopropoxy, cyclopropoxymethyl, cyclopropylmethoxy, cyclopropylmethoxymethyl, cyclopropylmethoxyethyl, methoxymethyl, ethoxymethyl, methoxyethyl, ethoxyethyl, vinyl, prop-2-enyl, but-3-enyl, and pent-4-enyl.

3. The compound of claim 1 or a pharmaceutically acceptable salt thereof, wherein:
each R^{1a} substituent is independently selected from C₁₋₆ alkyl, amino, cyano, halo, and hydroxy;
each R^{2a} substituent is independently selected from hydrogen, hydroxy, methyl, ethyl, methoxy, ethoxy, fluoro;
R^{2b} is selected from phenyl, benzyl, biphenyl, naphthyl, pyridyl, pyridazinylpyrimidyl, pyrazinyl, imidazolyl, pyrazolyl, oxazolyl, isoxazolyl, thiazolyl, isothiazolyl, oxadiazolyl, and bicyclo[1.1.1]pentyl; and
each R^{2c} substituent is independently selected from aminomethyl, hydroxy, methoxy, ethoxy, difluoromethoxy, trifluoromethoxy, fluoromethyl, difluoromethyl, trifluoromethyl, trifluoroethyl, methyl, ethyl, n-propyl, isopropyl, cyclopropyl, n-butyl, tert-butyl, fluoro, chloro, bromo, iodo, aminoethoxy, N-methylaminoethoxy, N-ethylaminoethoxy, N,N-dimethylaminoethoxy, 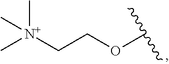 [SMILES: *OCC[N+](C)(C)C]
carboxy, carboxymethoxy, (carboxymethoxy)methyl, aminocarbonyl, N,N-dimethylaminocarbonyl, and aminocarbonylmethyl.

3. The compound of claim 1 or a pharmaceutically acceptable salt thereof, wherein
R^{3a} is selected from hydrogen, methyl, ethyl, n-propyl, isopropyl, n-butyl, 2-methylpropyl, tert-butyl, cyclopropyl, cyclopropylmethyl, cyclopropylethyl, cyclobutyl, cyclobutylmethyl, cyclobutylethyl, aminoethyl, aminopropyl, aminobutyl, hydroxyethyl, hydroxypropyl, hydroxybutyl, carboxymethyl, carboxyethyl, carboxypropyl, carboxybutyl, thioethyl, and thiopropyl, wherein R^{3a} may be substituted by 0, 1, or 2 R^{3c} substituents each independently selected from fluoro, chloro, methyl, ethyl, n-propyl, isopropyl, cyclopropyl, n-butyl, sec-butyl, tert-butyl, amino, aminomethyl, N-methylamino, N-methylaminomethyl, N-ethylamino, N-ethylaminomethyl, N,N-dimethylamino, N,N-dimethylaminomethyl, N,N-diethylamino, N,N-diethylaminomethyl, N,N,N-trimethylammonium, N,N,N-trimethylmethylammonium, hydroxy, hydroxymethyl, —SO₂CH₃, —CH₂SO₂CH₃, —CH₂CH₂SO₂CH₃, cyano, cyanomethyl, methoxy, ethoxy, methoxymethyl, methoxyethyl, carboxy, carboxymethyl, and carboxyethyl; and
R^{3b} is selected from hydrogen, methyl, ethyl, n-propyl, isopropyl, n-butyl, isobutyl, sec-butyl, tert-butyl, n-pentyl, isopentyl, neopentyl, cyclopropyl, cyclobutyl, cyclopentyl, cyclohexyl, cyclopropylmethyl, cyclobutylmetyl, cyclopentylmethyl, cyclohexylmethyl, bicyclo[1.1.1]pentylmethyl, hydroxymethyl, 1-hydroxyethyl, 2-hydroxyethyl, hydroxypropyl, 3-hydroxypropyl, 1-methyl-1-hydroxyethyl, hydroxyisopropyl, hydroxybutyl, methoxymethyl, methoxyethyl, methoxypropyl, ethoxymethyl, ethoxyethyl, aminomethyl, 2-aminoethyl, N-methylaminomethyl, N,N-dimethylaminomethyl, N-methylaminoethyl, N,N-dimethylaminoethyl, N-methylaminopropyl, N,N-dimethylaminopropyl, 1-aminopropyl, 2-aminopropyl, 3-aminopropyl, 2-aminoprop-2-yl, fluoromethyl, difluoromethyl, trifluoromethyl, fluoroethyl, difluoroethyl, trifluoroethyl, benzyl, 3-pyridinylmethyl, 4-pyridinylmethyl, imidazolylmethyl, thiazolylmethyl, oxazolylmethyl, thiophenylmethyl, furanylmethyl, pyrazolylmethyl, N-pyrazolylmethyl, 1-phenylethyl, 1-(4-pyridinyl)ethyl, aminocarbonylmethyl, aminocarbonylethyl, aminocarbonylpropyl, (N,N-dimethyl)aminocarbonylmethyl, (N,N-dimethyl)aminocarbonylethyl, thiomethyl, thioethyl, thiopropyl, —CH₂CH₂SO₂CH₃, carboxymethyl, carboxyethyl, 2-carboxyethyl, carboxypropyl, 3-carboxypropyl, carboxybutyl, 4-carboxybutyl, piperazinylmethyl, morpholinomethyl, piperidinylmethyl, azetidinylmethyl, tetrahydropyranylmethyl, aminocarbonylaminomethyl, aminocarbonylaminoethyl, aminocarbonylaminopropyl, and aminocarbonylaminobutyl, wherein R^{3b} may be substituted by 0, 1, or 2 R^{3c} substituents each independently selected from fluoro, chloro, methyl, ethyl, n-propyl, isopropyl, cyclopropyl, n-butyl, sec-butyl, tert-butyl, amino, aminomethyl, N-methylamino, N-methylaminomethyl, N-ethylamino, N-ethylaminomethyl, N,N-dimethylamino, N,N-dimethylaminomethyl, N,N-diethylamino, N,N-diethylaminomethyl, N,N,N-trimethylammonium, N,N,N-trimethylmethylammonium, hydroxy, hydroxymethyl, —SO₂CH₃, —CH₂SO₂CH₃, —CH₂CH₂SO₂CH₃, cyano, cyanomethyl, methoxy, ethoxy, methoxymethyl, methoxyethyl, carboxy, carboxymethyl, and carboxyethyl.

5. The compound of claim 1 or a pharmaceutically acceptable salt thereof, wherein R^{3a} and R^{3b}, together with the atoms to which they are attached, form a saturated ring system substituted by 0, 1, or 2, R^{3d} substituents, said saturated ring system is selected from:

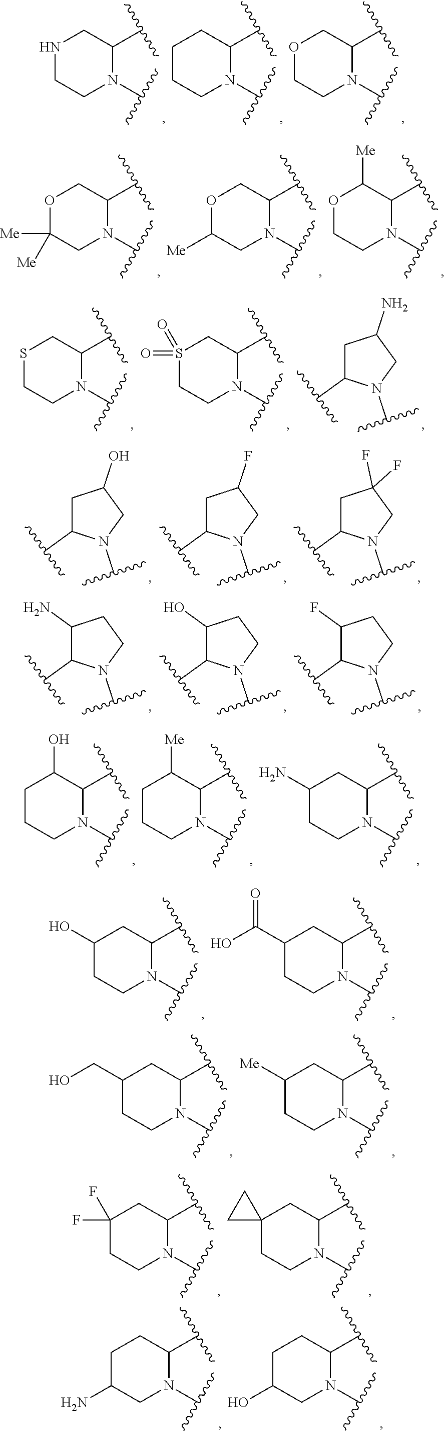

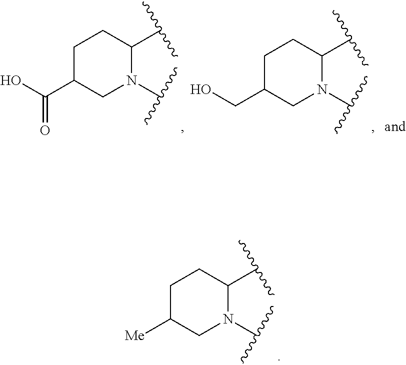 [SMILES: *C1CCC(C(=O)O)CN1* ; *C1CCC(CO)CN1* ; *C1CCC(C)CN1*]

6. The compound of claim 1 or a pharmaceutically acceptable salt thereof, wherein
R^{4a} is selected from hydrogen, methyl, ethyl, propyl, hydroxy, methoxy, and fluoro;
R^{4b} is selected from indolyl, naphthyl, quinolinyl, pyrrolo[2,3-b]pyridinyl, [1,2,4]triazolo[1,5-a]pyridine, 1H-pyrazolo[3,4-b]pyridine, indazolyl, benzothiazolyl, and benzothiophenyl, wherein R^{4b} substituted with 0, 1, or 2 R^{4c} substituents each independently selected from fluoro, chloro, bromo, iodo, cyano, nitro, hydroxy, methyl, ethyl, propyl, isopropyl, carboxy, carboxymethyl, and carboxyethyl;
R^{5a} is selected from hydrogen, methyl, ethyl, n-propyl, isopropyl, cyclopropyl, n-butyl, isobutyl, cyclobutyl, n-pentyl, isopentyl, neopentyl, cyclopentyl, cyclobutyl, cyclopropylmethyl, cyclopropylethyl, cyclobutylmethyl, cyclobutylethyl, cyclopentylmethyl, cyclopentylethyl, cyclohexylmethyl, cyclohexylethyl, phenyl, benzyl, phenylethyl, phenylpropyl, oxazolylmethyl, thiazolylmethyl, imidazolylmethyl, triazolylmethyl, oxadiazolylmethyl, thiadiazolylmethyl, oxazolylethyl, thiazolylethyl, imidazolylethyl, triazolylethyl, oxadiazolylethyl, thiadiazolylethyl, oxazolylpropyl, thiazolylpropyl, imidazolylpropyl, triazolylpropyl, oxadiazolylpropyl, thiadiazolylpropyl, azetidinylmethyl, azetidinylethyl, oxetanylmethyl, oxetanylmethyl, pyrrolidinylmethyl, pyrrolidinylethyl, tetrahydrofuranylmethyl, tetrahydrofuranylethyl, piperidinylmethyl, piperidinylethyl, piperazinylmethyl, piperazinylethyl, tetrahydropyranylmethyl, tetrahydropyranylmethyl, hydroxyethyl, hydroxypropyl, hydroxyisopropyl, hydroxybutyl, 3-hydroxy-2,2-dimethylpropyl, cyclopropylmethyl, 1-hydroxypropan-2-yl, 2-hydroxyethyl, 3-hydroxypropyl, 2-hydroxyisopropyl, methoxyethyl, methoxypropyl, ethoxyethyl, ethoxypropyl, cyanomethyl, cyanoethyl, cyanopropyl, cyanobutyl, carboxymethyl, carboxyethyl, carboxypropyl, carboxybutyl, 2-fluoroethyl, 2,2-difluoroethyl, 2,2,2-trifluoroethyl, 2-aminoethyl, 3-aminopropyl, 3-amino-2,2-dimethylpropyl, cyclopropylmethyl, 4-aminobutyl, aminomethylcarbonylaminoethyl, aminoethylcarbonylaminoethyl, aminomethylcarbonylaminopropyl, aminoethylaminocarbonylmethyl, aminoethylaminocarbonylethyl, aminoethylaminocarbonylpropyl, aminohexylcarbonylaminoethyl, aminohexylcarbonylaminoethyl, (N-methylamino)ethyl, (N-methylamino)propyl, (N-ethylamino)ethyl, (N,N-diethylamino)propyl, (N,N-dimethylamino)ethyl, (N,N-dimethylamino)propyl, (N,N-diethylamino)ethyl, (N,N-diethylamino)propyl, (N,N,N-trimethylammonium)ethyl, (N,N,N-trimethylammonium)propyl, (N,N,N-triethylammonium)ethyl, (N,N,N-triethylammonium)propyl, (N-methylamino)methylcarbonylaminoethyl, (N-methylamino)ethylcarbonylaminoethyl, (N-methylamino)methylcarbonylaminopropyl, (N-ethylamino)methylcarbonylaminoethyl, (N-ethylamino)ethylcarbonylaminoethyl, (N-ethylamino)methylcarbonylaminopropyl, (N-methylamino)pentylcarbonylaminoethyl, (N-methylamino)pentylcarbonylaminoethyl, (N-methylamino)pentylcarbonylaminopropyl, (N,N-dimethylamino)methylcarbonylaminoethyl, (N,N-dimethylamino)ethylcarbonylaminoethyl, (N,N-dimethylamino)methylcarbonylaminopropyl, (N,N-diethylamino)methylcarbonylaminoethyl, (N,N-diethylamino)ethylcarbonylaminoethyl, (N,N-diethylamino)methylcarbonylaminopropyl, (N,N-dimethylamino)pentylcarbonylaminoethyl, (N,N-dimethylamino)pentylcarbonylaminoethyl, (N,N-dimethylamino)pentylcarbonylaminopropyl, N,N,N-trimethyl-ethan-1-ammonium, N,N,N-trimethyl-propan-1-ammonium, (N,N,N-trimethylammonium)methylcarbonylaminoethyl, (N,N,N-trimethylammonium)ethylcarbonylaminoethyl, (N,N,N-trimethylammonium)methylcarbonylaminopropyl, (N,N,N-trimethylammonium)ethylcarbonylaminopropyl, (N,N,N-trimethylammonium)pentylcarbonylaminoethyl, (N,N,N-trimethylammonium)pentylcarbonylaminopropyl, (carboxymethyl)oxyethyl, and (carboxymethyl)oxypropyl, wherein R^{5a} is substituted by 0, 1, 2, or 3 R^{5d} substituents each independently selected from chloro, fluoro, hydroxy, hydroxymethyl, hydroxyethyl, methyl, ethyl, n-propyl, isopropyl, n-butyl, isobutyl, cyclobutyl, n-pentyl, isopentyl, neopentyl, carboxy, carboxymethyl, carboxyethyl, methoxy, ethoxy, methoxymethyl, methoxyethyl, methoxypropyl, amino, aminomethyl, aminoethyl, N-methylamino, (N-methylamino)methyl, (N-methylamino)ethyl, N,N-dimethylamino, (N,N-dimethylamino)methyl, (N,N-dimethylamino)ethyl, N,N-diethylamino, (N,N-diethylamino)methyl, (N,N-diethylamino)ethyl, aminomethylcarbonylamino, aminoethylcarbonylamino, aminopentylcarbonylamino, aminomethylcarbonylaminomethyl, aminoethylcarbonylaminomethyl, (N-methylamino)methylcarbonylamino, (N-methylamino)ethylcarbonylamino, (N-methylamino)methylcarbonylaminomethyl, (N-methylamino)ethylcarbonylaminomethyl, (N,N-dimethylamino)methylcarbonylamino, (N,N-dimethylamino)ethylcarbonylamino, (N,N-dimethylamino)methylcarbonylaminomethyl, (N,N-dimethylamino)ethylcarbonylaminomethyl, (N,N-diethylamino)methylcarbonylamino, (N,N-diethylamino)ethylcarbonylamino, (N,N-diethylamino)methylcarbonylaminomethyl, (N,N-diethylamino)ethylcarbonylaminomethyl, N,N,N-trimethylammonium, (N,N,N-trimethylammonium)methyl, (N,N,N-trimethylammonium)ethyl, (N,N,N-trimethylammonium)ethoxy, (N,N,N-trimethylammonium)ethoxymethyl, (N,N,N-trimethylammonium)methylcarbonylamino, (N,N,N-triethylammonium)methylcarbonylamino, (N,N,N-trimethylammonium)ethylcarbonylamino, (N,N,N-trimethylammonium)pentylcarbonylamino, (N,N,N-trimethylammonium)methylcarbonylaminomethyl, (N,N,N-trimethylammonium)ethylcarbonylaminomethyl, cyano, cyanomethyl, cyanoethyl, tetrazoyl, tetrazoylmethyl, tetrazoylethyl, carboxymethoxy, carboxyethoxy, carboxymethoxymethyl, and carboxyethoxymethyl;
R^{5b} is selected from hydrogen, methyl, ethyl, isopropyl, n-propyl, cyclopropyl, isobutyl, n-butyl, sec-butyl, isobutyl, tert-butyl, cyclobutyl, cyclopropylmethyl, oxetanylmethyl, tetrahydrofurylmethyl, tetrahydropyranylmethyl, hydroxymethyl, hydroxyethyl, hydroxypropyl, methoxymethyl, methoxyethyl, methoxypropyl, carboxymethyl, carboxyethyl, carboxypropyl, carboxybutyl, (carboxymethoxy)methyl, (carboxymethoxy)ethyl, (carboxyethoxy)methyl, (carboxyethoxy)ethyl, aminomethyl, aminoethyl, aminopropyl, aminobutyl, (N-methylamino)methyl, (N-methylamino)ethyl, (N-methylamino)propyl, (N-methylamino)butyl, (N,N-dimethylamino)methyl, (N,N-dimethylamino)ethyl, (N,N-dimethylamino)propyl, (N,N-dimethylamino)butyl, (N,N-diethylamino)methyl, (N,N-diethylamino)ethyl, (N,N-diethylamino)propyl, (N,N-diethylamino)butyl, N,N,N-trimethylmethan-1-ylammonium, N,N,N-trimethylethan-1-ylammonium, N,N,N-triethylethan-1-ylammonium, N,N,N-trimethylpropan-1-ylammonium, N,N,N-trimethylbutan-1-ylammonium, (N,N,N-trimethylammonium)ethoxymethyl, (N,N,N-trimethylammonium)ethoxyethyl, (N,N,N-triethylammonium)ethoxyethyl, (N,N,N-trimethylammonium)ethoxypropyl, (N,N,N-trimethylammonium)ethoxybutyl, aminocarbonylmethyl, aminocarbonylethyl, aminocarbonylpropyl, aminocarbonylbutyl, (N-methylamino)carbonylmethyl, (N-methylamino)carbonylethyl, (N-methylamino)carbonylpropyl, (N-methylamino)carbonylbutyl, (N,N-dimethylamino)carbonylmethyl, (N,N-dimethylamino)carbonylethyl, (N,N-dimethylamino)carbonylpropyl, (N,N-dimethylamino)carbonylbutyl, aminomethylcarbonylaminoethyl, aminomethylcarbonylaminopropyl, aminoethylcarbonylaminoethyl, aminoethylcarbonylaminopropyl, (N,N-dimethylamino)methylcarbonylaminoethyl, (N,N-dimethylamino)methylcarbonylaminopropyl, (N,N-dimethylamino)ethylcarbonylaminoethyl, (N,N-dimethylamino)ethylcarbonylaminopropyl, (N,N,N-trimethylammonium)methylcarbonylaminoethyl, (N,N,N-triethylammonium)methylcarbonylaminoethyl, (N,N,N-trimethylammonium)methylcarbonylaminopropyl, (N,N,N-trimethylammonium)ethylcarbonylaminoethyl, (N,N,N-trimethylammonium)ethylcarbonylaminopropyl, (N,N,N-trimethylammonium)ethoxyethyl, (N,N,N-trimethylammonium)ethoxypropyl, 2-fluoroethyl, 2,2-difluoroethyl, 2,2,2-trifluoroethyl, 3,3,3-trifluoropropyl, cyanomethyl, cyanoethyl, cyanopropyl, cyanoisopropyl, and cyanobutyl, wherein R^{5b} is substituted by 0, 1, 2, or 3 R^{5e} substituents each independently selected from chloro, fluoro, hydroxy, hydroxymethyl, hydroxyethyl, methyl, ethyl, n-propyl, isopropyl, n-butyl, isobutyl, cyclobutyl, n-pentyl, isopentyl, neopentyl, carboxy, carboxymethyl, carboxyethyl, methoxy, ethoxy, methoxymethyl, methoxyethyl, methoxypropyl, amino, aminomethyl, aminoethyl, N-methylamino, (N-methylamino)methyl, (N-methylamino)ethyl, N,N-dimethylamino, (N,N-dimethylamino)methyl, (N,N-dimethylamino)ethyl, N,N-diethylamino, (N,N-diethylamino)methyl, (N,N-diethylamino)ethyl, aminomethylcarbonylamino, aminoethylcarbonylamino, aminopentylcarbonylamino, aminomethylcarbonylaminomethyl, aminoethylcarbonylaminomethyl, (N-methylamino)methylcarbonylamino, (N-methylamino)ethylcarbonylamino, (N-methylamino)methylcarbonylaminomethyl, (N-methylamino)ethylcarbonylaminomethyl, (N,N-dimethylamino)methylcarbonylamino, (N,N-dimethylamino)ethylcarbonylamino, (N,N-dimethylamino)methylcarbonylaminomethyl, (N,N-dimethylamino)ethylcarbonylaminomethyl, (N,N-diethylamino)methylcarbonylamino, (N,N-diethylamino)ethylcarbonylamino, (N,N-diethylamino)methylcarbonylaminomethyl, (N,N-diethylamino)ethylcarbonylaminomethyl, N,N,N-trimethylammonium, (N,N,N-trimethylammonium)methyl, (N,N,N-trimethylammonium)ethyl, (N,N,N-trimethylammonium)ethoxy, (N,N,N-trimethylammonium)ethoxymethyl, (N,N,N-trimethylammonium)methylcarbonylamino, (N,N,N-triethylammonium)methylcarbonylamino, (N,N,N-trimethylammonium)ethylcarbonylamino, (N,N,N-trimethylammonium)pentylcarbonylamino, (N,N,N-trimethylammonium)methylcarbonylaminomethyl, (N,N,N-trimethylammonium)ethylcarbonylaminomethyl, cyano, cyanomethyl, cyanoethyl, tetrazoyl, tetrazoylmethyl, tetrazoylethyl, carboxymethoxy, carboxyethoxy, carboxymethoxymethyl, and carboxyethoxymethyl; and
R^{5c} is hydrogen, methyl, or ethyl.

7. The compound of claim 1 or a pharmaceutically acceptable salt thereof, wherein
R^{5a} and R^{5b}, together with the atoms to which they are attached, form a saturated mono- or bi-cyclic ring system substituted with 0, 1, 2, or 3 R^{5d} and 0, 1, 2, or 3 R^{5e} substituents, wherein said mono- or bi-cyclic ring system is selected from:

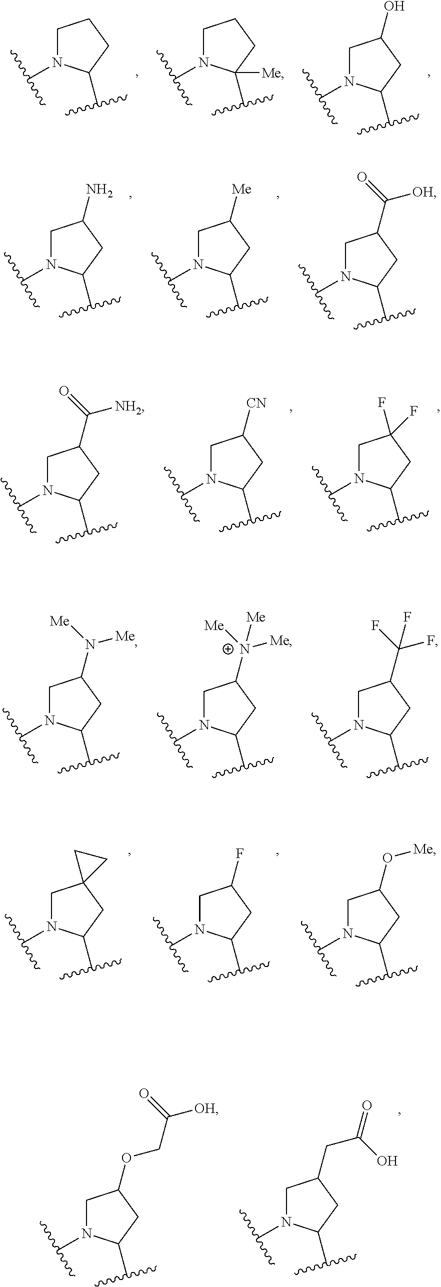

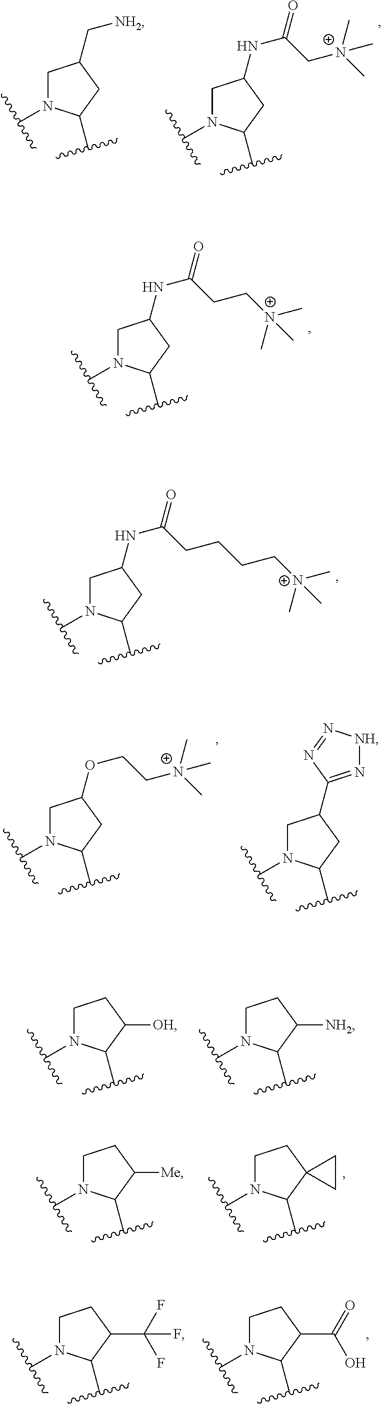

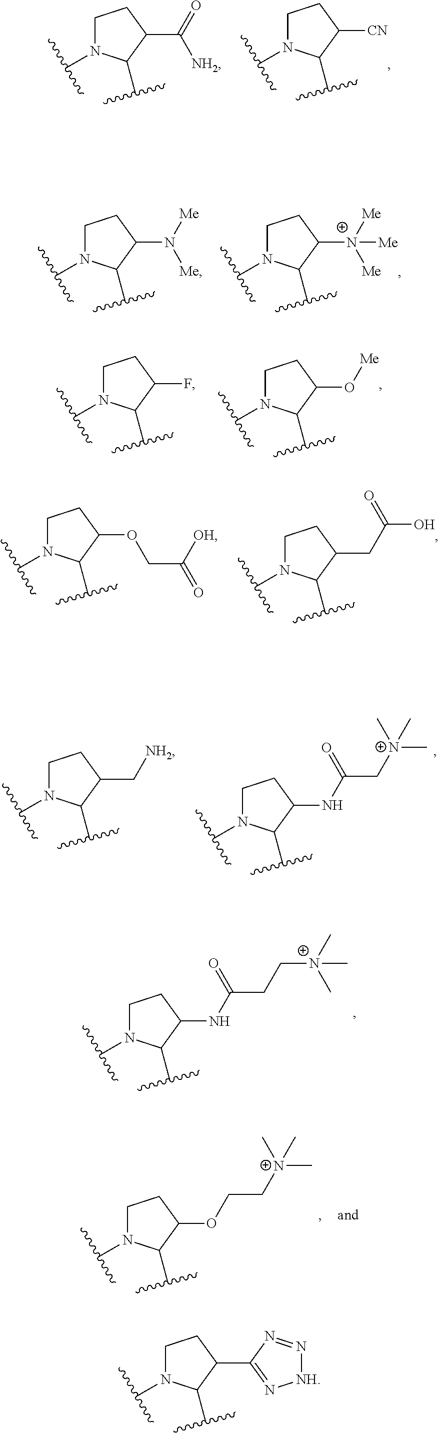

8. The compound of claim 1 or a pharmaceutically acceptable salt thereof, wherein
R^{6a} is selected from hydrogen, hydroxy, methyl, ethyl, propyl, and methoxy;
R^{6b} is selected from hydrogen, hydroxy, methyl, ethyl, propyl, methoxy, and ethoxy;
R^{6c} is selected from aminocarbonyl, (aminocarbonyl)methyl, (aminocarbonyl)ethyl, (aminocarbonyl)propyl, (N-methylamino)carbonyl, (N-methylamino)carbonylmethyl, (N-methylamino)carbonylethyl, (N-methylamino)carbonylpropyl, (N,N-dimethylamino)carbonyl, (N,N-dimethylamino)carbonylmethyl, (N,N-dimethylamino)carbonylethyl, (N,N-dimethylamino) carbonylpropyl, (N,N-diethylamino)carbonyl, (N,N-diethylamino)carbonylmethyl, (N,N-diethylamino)carbonylethyl, (N,N-diethylamino)carbonylpropyl, aminocarbonylamino, (aminocarbonylamino)methyl, (aminocarbonylamino)ethyl, (aminocarbonylamino)propyl, methoxy, methoxymethyl, methoxyethyl, ethoxy, ethoxymethyl, ethoxyethyl, methylsulfonyl, (methylsulfonyl)methyl, (methylsulfonyl)ethyl, (methylsulfonyl)propyl, amino, aminomethyl, aminoethyl, aminopropyl, aminoisopropyl, aminobutyl, carboxy, carboxymethyl, carboxyethyl, hydroxy, hydroxymethyl, hydroxyethyl, hydroxypropyl, hydroxyisopropyl, trifluoromethyl, 2,2,2-trifluoroethyl, methyl, ethyl, isopropyl, n-propyl, isobutyl, n-butyl, sec-butyl, isobutyl, tert-butyl, phenyl, and benzyl;
R^{7a} is selected from hydrogen, methyl, ethyl, hydroxy, methoxy, and ethoxy;
R^{7b} is selected from phenyl, biphenyl, naphthyl, pyridyl, pyridazinyl, pyrimidyl, pyrazinyl, imidazolyl, pyrazolyl, furyl, thiophenyl, oxazolyl, isoxazolyl, thiazolyl, isothiazolyl, oxadiazolyl, 3-oxoisoindolinyl, and bicyclo[1.1.1]pentyl;
R^{7c} is selected from fluoro, chloro, bromo, iodo, hydroxy, methoxy, ethoxy, difluoromethoxy, trifluoromethoxy, carboxy, carboxymethyl, methoxycarbonyl, methoxycarbonylmethyl, ethoxycarbonyl, carboxymethoxy, carboxyethoxy, carboxymethoxymethyl, aminocarbonyl, aminocarbonylmethyl, (N,N-dimethylamino)carbonyl, (N,N-dimethylamino)carbonylmethyl, amino, aminomethyl, N,N-dimethylamino, (N,N-dimethylamino)methyl, (N,N-diethylamino)methyl, N,N,N-trimethylammonium, N,N,N-trimethylmethylammonium, N,N,N-triethylmethylammonium, methyl, ethyl, propyl, isopropyl, difluoromethyl, trifluoromethyl, —SO₂OH, —CH₂SO₂OH, —SO₂NH₂, —CH₂SO₂NH₂, aminoethoxy, aminoethoxymethyl, (N,N-dimethylamino)ethoxy, N,N,N-trimethyleth-1-oxy-ammonium, aminocarbonylamino, and aminocarbonylaminomethyl;
R^{8a} is selected from hydrogen, methyl, ethyl, hydroxy, methoxy, and ethoxy;
R^{8b} is selected from indolyl, naphthyl, indolylmethyl, naphthylmethyl, quinolinyl, pyrrolo[2,3-b]pyridinyl, indazolyl, benzothiazolyl, and benzothiophenyl;
each R^{8c} independently is selected from methyl, ethyl, isopropyl, fluoro, chloro, bromo, iodo, cyano, amino, aminomethyl, nitro, hydroxy, hydroxymethyl, carboxy, fluoromethyl, difluoromethyl, trifluoromethyl, trichloromethyl, methoxy, ethoxy, and trifluoromethoxy; and
R⁹ is selected from hydrogen, methyl, ethyl, and propyl.

9. The compound of claim 1 or a pharmaceutically acceptable salt thereof, wherein
R^{10a} is selected from hydrogen, hydroxy, methyl, ethyl, propyl, methoxy, ethoxy, and propoxy;
R^{10b} is selected from phenyl, benzyl, biphenyl, biphenylmethyl, pyridyl, pyridylmethyl, pyridazinyl, pyrimidyl, pyrazinyl, imidazolyl, imidazolylmethyl, pyrazolyl, furyl, furylmethyl, oxazolyl, oxazolylmethyl, thiazolyl, indolyl, [1,2,4]triazolo[1,5-a]pyridine, and bicyclo[1.1.1]pentyl;
R^{10c} is independently selected from methyl, ethyl, n-propyl, isopropyl, cyclopropyl, n-butyl, isobutyl, cyclobutyl, n-pentyl, isopentyl, neopentyl, cyclopropylmethyl, cyclopropylethyl, cyclobutylmethyl, cyclopentyl, cyclohexyl, cyclopentylmethyl, cyclohexylmethyl, fluoromethyl, difluoromethyl, trifluoromethyl, 2,2,2-trifluoroethyl, trifluoromethoxy, 2,2,2-trifluoroethoxy, fluoro, chloro, bromo, iodo, hydroxy, hydroxymethyl, hydroxyethyl, cyano, amino, aminomethyl, aminoethyl, N-methylamino, (N-methylamino)methyl, (N-methylamino)ethyl, N,N-dimethylamino, (N,N-dimethylamino)methyl, (N,N-dimethylamino)ethyl, N,N,N-trimethylammonium, N,N,N-trimethylmethan-ylammonium, aminocarbonyl, aminocarbonylmethyl, aminocarbonylethyl, (N-methylamino)carbonyl, (N-methylamino) carbonylmethyl, (N,N-dimethylamino)carbonyl, (N,N-dimethylamino)carbonylmethyl, aminocarbonylamino, aminocarbonylaminomethyl, methylcarbonylamino, methylcarbonylaminomethyl, carboxy, carboxymethyl, carboxyethyl, methoxycarboxy, carboxymethoxy, carboxyethoxy, carboxymethoxymethyl, carboxyethoxymethyl, —S(═O)₂OH, —CH₂(S(═O)₂OH), —S(═O)₂NH₂, —CH₂(S(═O)₂NH₂), aminoethoxy, aminopropoxy, (N-methylamino)ethoxy, (N-ethylamino)ethoxy, (N,N-dimethylamino)ethoxy, (N,N-diethylamino) ethoxy, (N,N,N-trimethylammonium)ethoxy, methoxy, ethoxy, methoxymethyl, ethoxymethyl, difluoromethoxy, trifluoromethoxy, and 2,2,2-trifluoroethoxy;
R^{11a} is selected from hydrogen, hydroxy, methyl, ethyl, methoxy, and ethoxy;
R^{11b} is selected from (H₂N—C(═NH)—NH)methyl, (H₂N—C(═NH)—NH)ethyl, (H₂N—C(═NH)—NH)propyl, (H₂N—C(═NH)—NH)butyl, phenyl, benzyl, pyridinyl, pyridinylmethyl, indolyl, indolylmethyl, pyridazinyl, pyridazinylmethyl, pyrimidyl, pyrimidylmethyl, pyrazinyl, pyrazinylmethyl, imidazolyl, imidazolylmethyl, pyrazolyl, pyrazolylmethyl, [1,2,4]triazolo[1,5-a]pyridine, oxazolyl, oxazolylmethyl, thiazolyl, and thiazolylmethyl;
each R^{11c} is independently selected from fluoro, chloro, bromo, iodo, (carboxymethyl)oxy, (carboxymethyl)oxymethyl, (carboxyethyl)oxy, hydroxy, hydroxymethyl, hydroxyethyl, methoxy, ethoxy, difluoromethoxy, trifluoromethoxy, carboxy, carboxymethyl, aminocarbonyl, aminocarbonylmethyl, (N-methylamino)carbonyl, (N-methylamino) carbonylmethyl, (N,N-dimethylamino)carbonyl, (N,N-dimethylamino)carbonylmethyl, amino, N-methylamino, N,N-dimethylamino, N,N-diethylamino, aminomethyl, (N,N-dimethylamino) methyl, (N,N-diethylamino)methyl, N,N,N-trimethylammonium, N,N,N-trimethylmeth-1-yl-ammonium, N,N,N-triethylammonium, N,N,N-triethylmeth-1-yl-ammonium, methyl, ethyl, isopropyl, cyclopropyl, cyclobutyl, difluoromethyl, trifluoromethyl, aminoethoxy, aminopropoxy, aminoethoxymethyl, (N,N-dimethylamino)ethoxy, N,N,N-trimethyleth-1-yloxy-ammonium, cyano, and methylcarbonylpiperazyl [(N-acetyl)piperazyl;
R^{12a} is selected from hydrogen, methyl, ethyl, propyl, cyclopropyl, and isopropyl; and
R^{12b} is selected from hydrogen, methyl, ethyl, and propyl.

10. The compound of claim 1 or a pharmaceutically acceptable salt thereof, wherein
R^{12a} and R^{12b}, together with the atoms to which they are attached form a saturated ring selected from

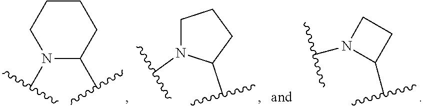 [SMILES: *C1CCCCN1* ; *C1CCCN1* ; *C1CCN1*]

11. The compound of claim 1 or a pharmaceutically acceptable salt thereof, wherein:
R^{13a} is hydrogen, methyl, or propyl;
R^{13b} is hydrogen or methyl;
R^{13c} is selected from indolyl, pyrrolo[2,3-b]pyridinyl, quinolinyl, indazolyl, and naphthyl;
R^{13c} is selected from indolyl, and pyrrolo[2,3-b]pyridinyl;
each R^{13d} independently is selected from fluoro, chloro, bromo, iodo, methyl, ethyl, propyl, isopropyl, carboxy, carboxymethyl, methoxy, difluoromethoxy, trifluoromethoxy, and ethoxy;
each R^{14a} independently is selected from amino, hydroxy, N-methylamino, N,N-dimethylamino, N-ethylamino, methoxy, and ethoxy; and
each R^{14b} independently is selected from hydrogen, methyl, ethyl, propyl, isopropyl, n-butyl, sec-butyl, isobutyl, n-pentyl, isopentyl, neopentyl, trifluoromethyl, trifluoroethyl, phenyl, benzyl, chlorophenyl, dichlorophenyl, fluorophenyl, difluorophenyl, bromophenyl, iodophenyl, chlorobenzyl, dichlorobenzyl, fluorobenzyl, difluorobenzyl, bromobenzyl, iodobenzyl, naphthyl, naphthylmethyl, pyrazolyl, pyrazolylmethyl, indolyl, indolylmethyl, imidazolyl, imidazolylmethyl, pyridyl, and pyridylmethyl.

12. The compound of claim 1 selected from the group consisting of SEQ ID NOS: 1-289, or a pharmaceutically acceptable salt thereof.

13. The compound of claim 1 selected from the group consisting of (SEQ ID NOS 5, 10, 18, 22, 30, 31, 41, 45, 48, 51, 52, 53, 60, 68, 77, 83, 87, 104, 183, 196, 206, 210, 214, 221, 229, 237, 247, 250, 254, 257, and 265 respectively, in order of appearance): 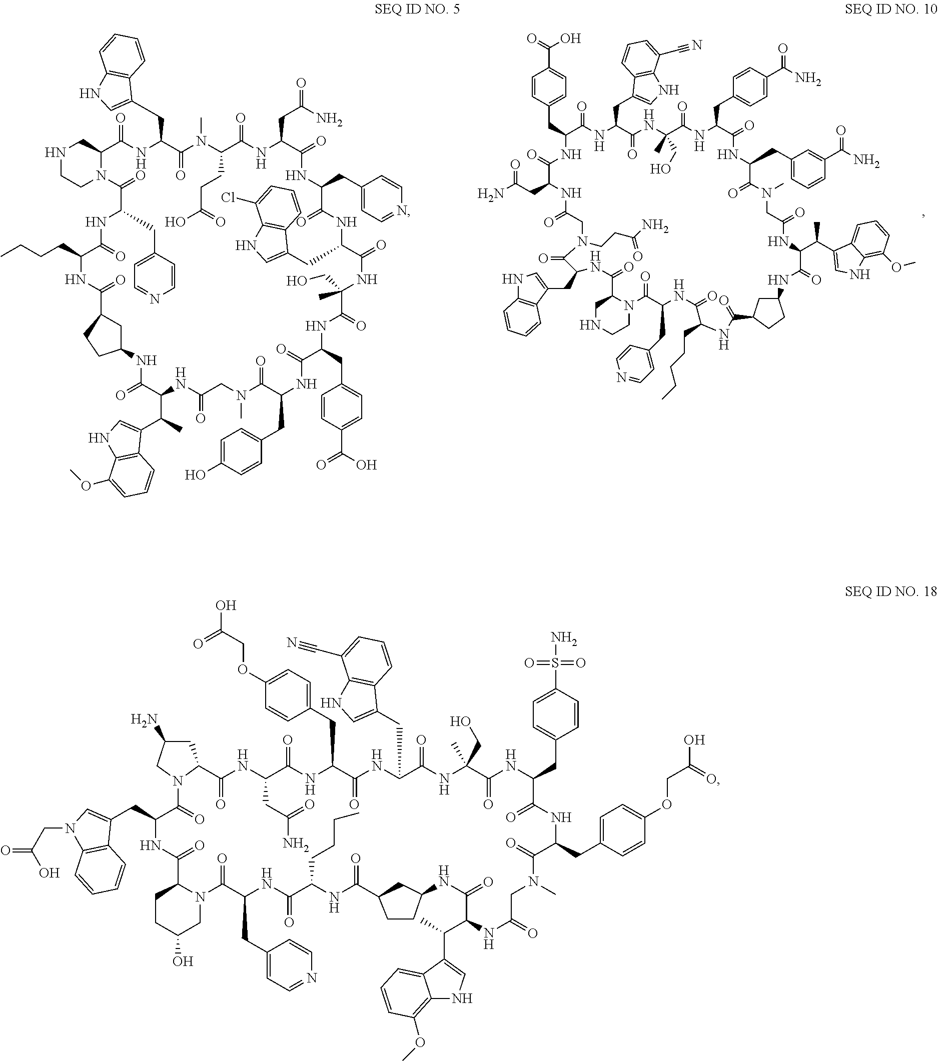 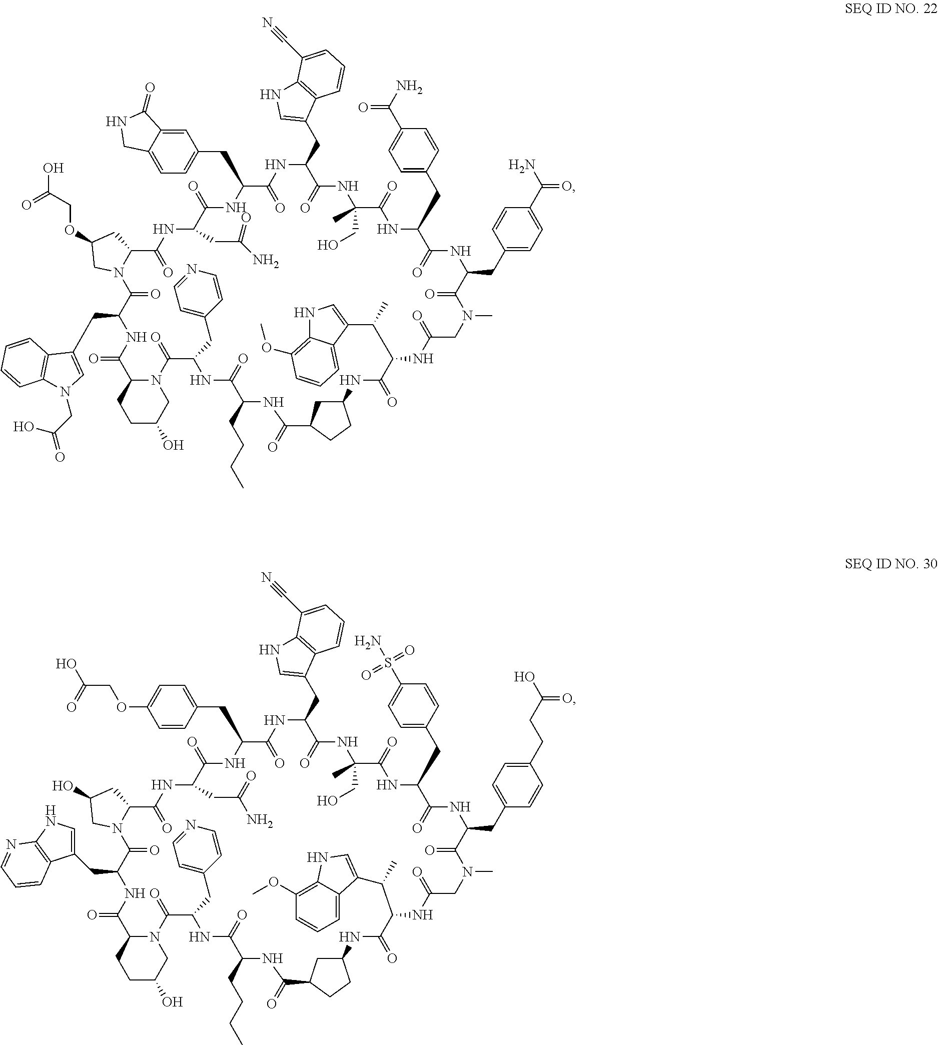 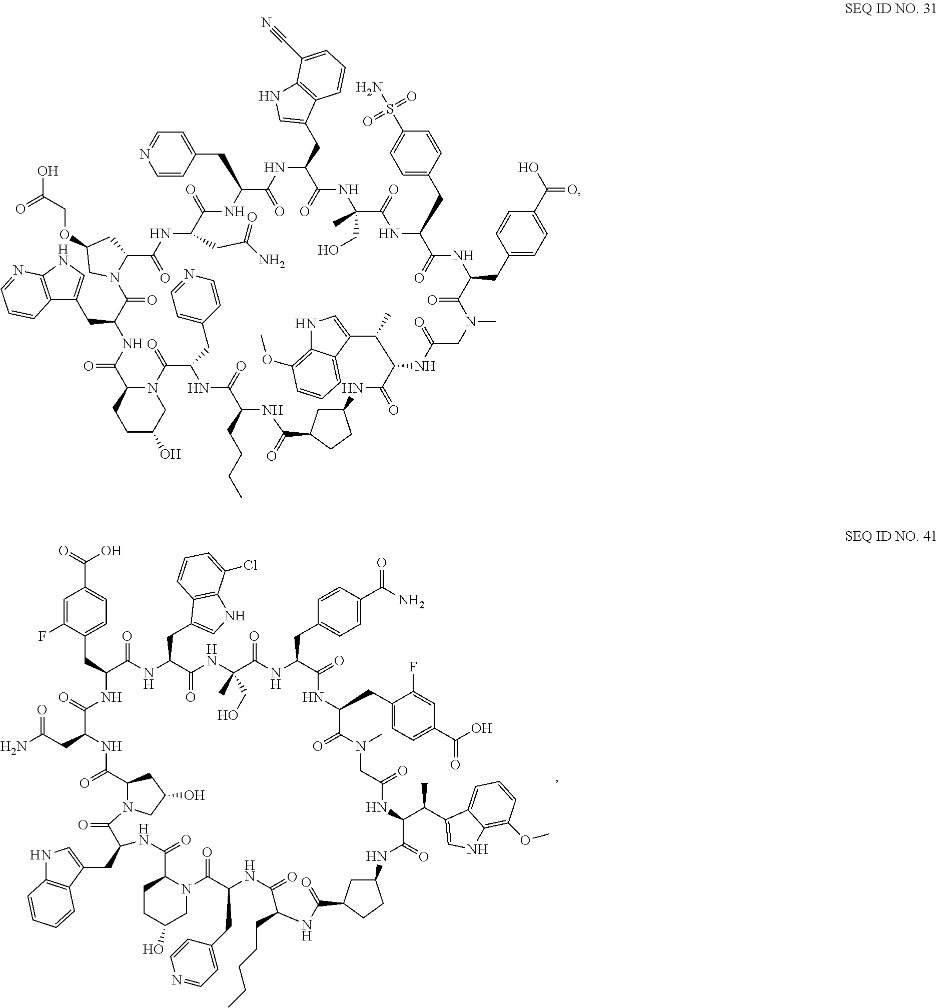 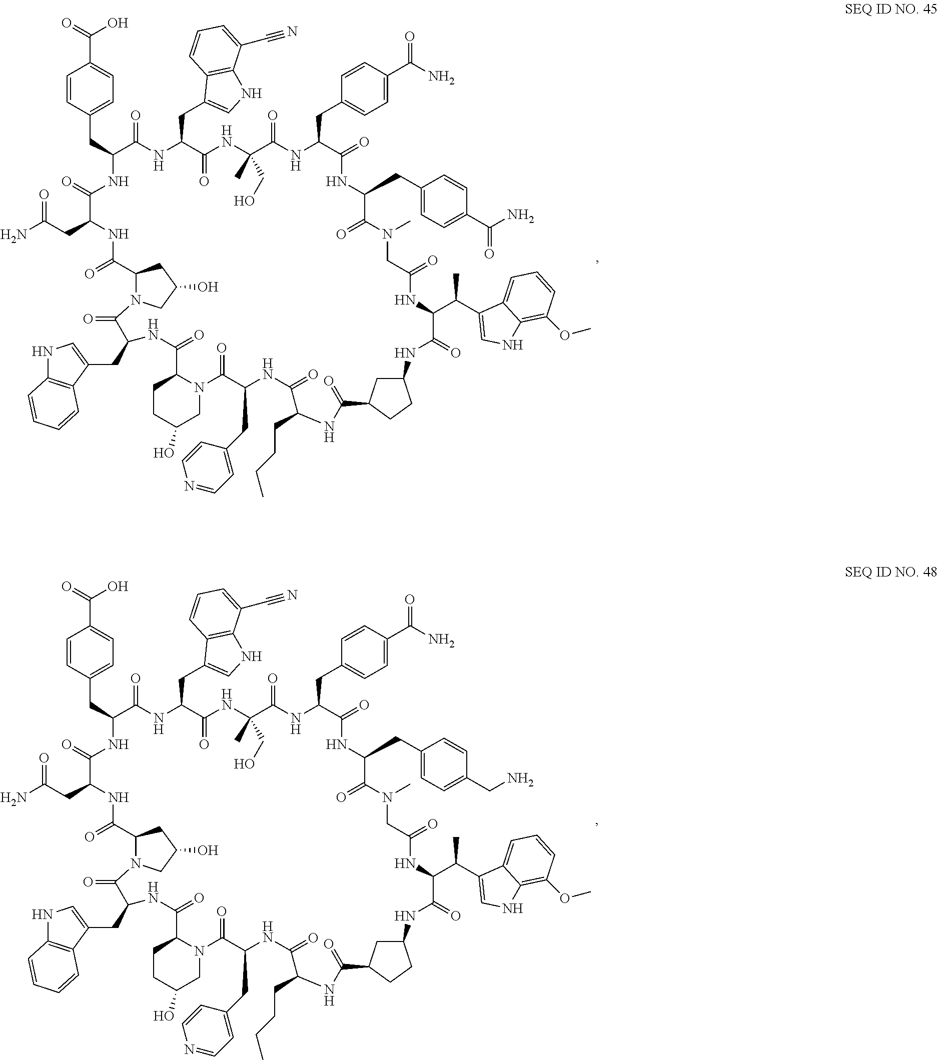 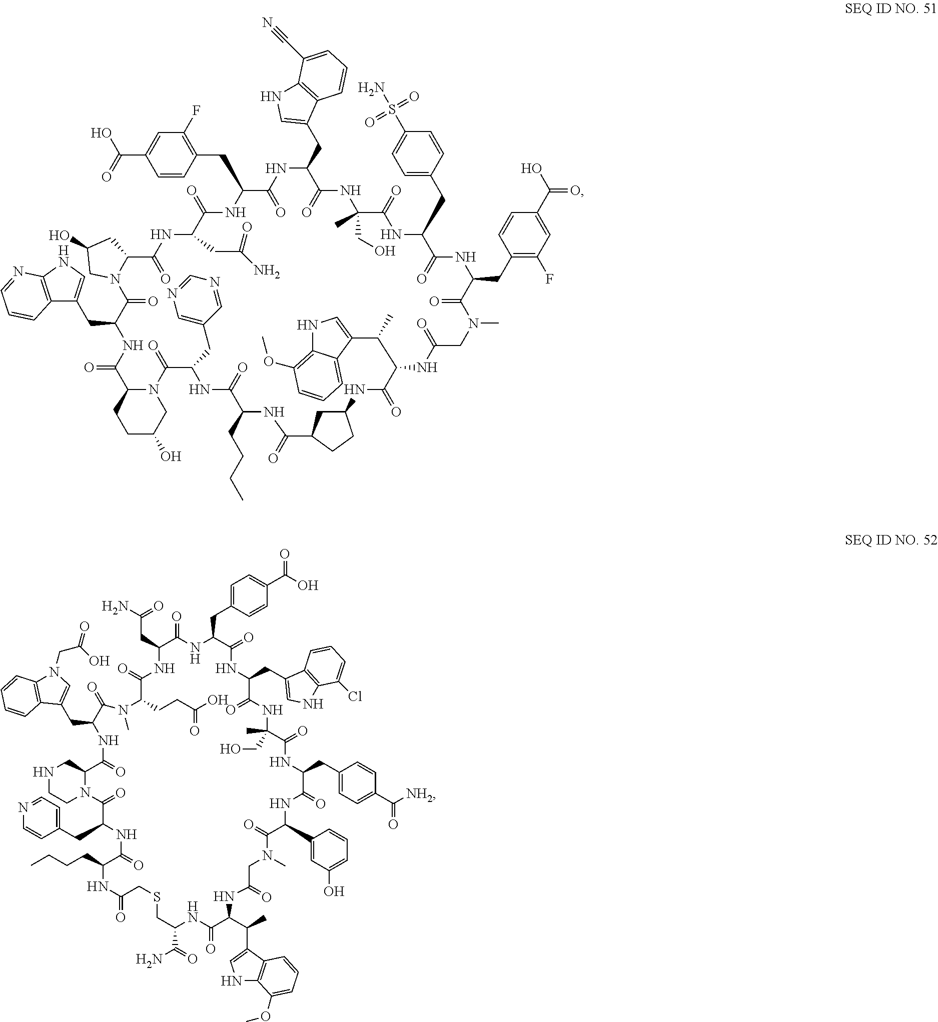 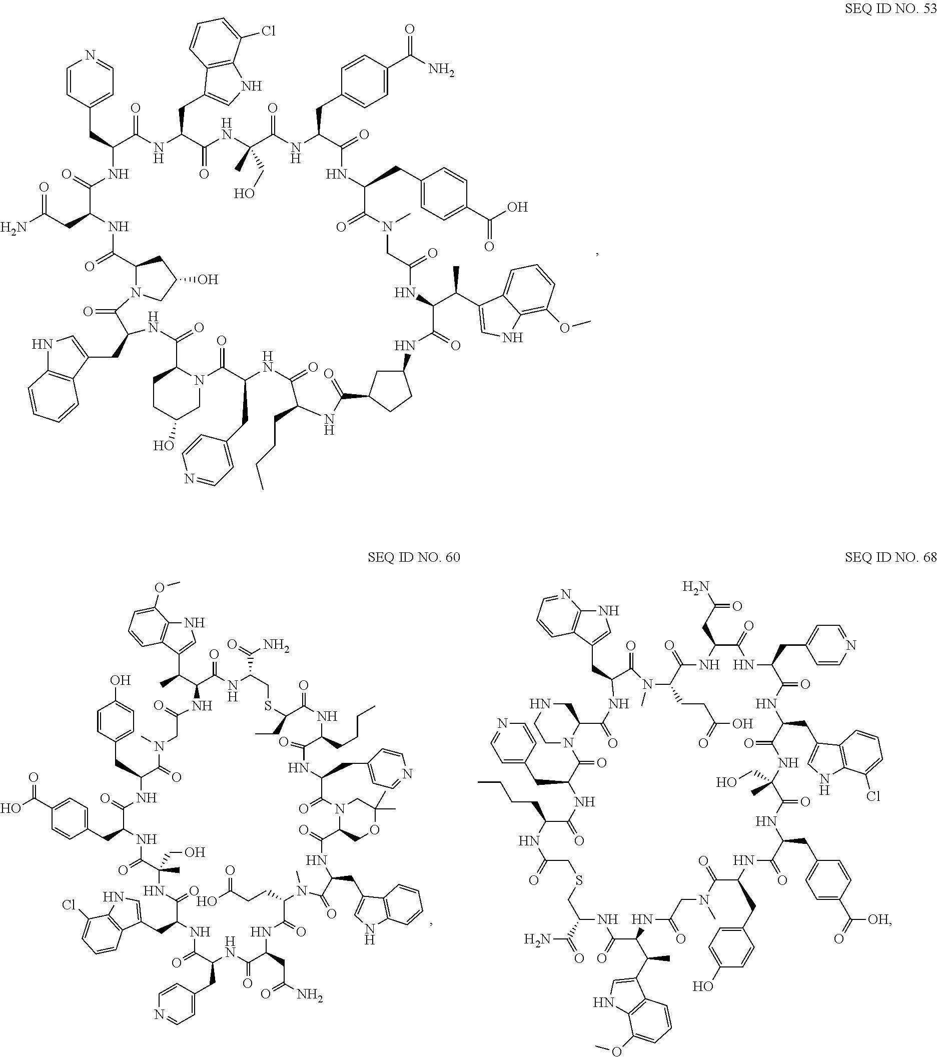 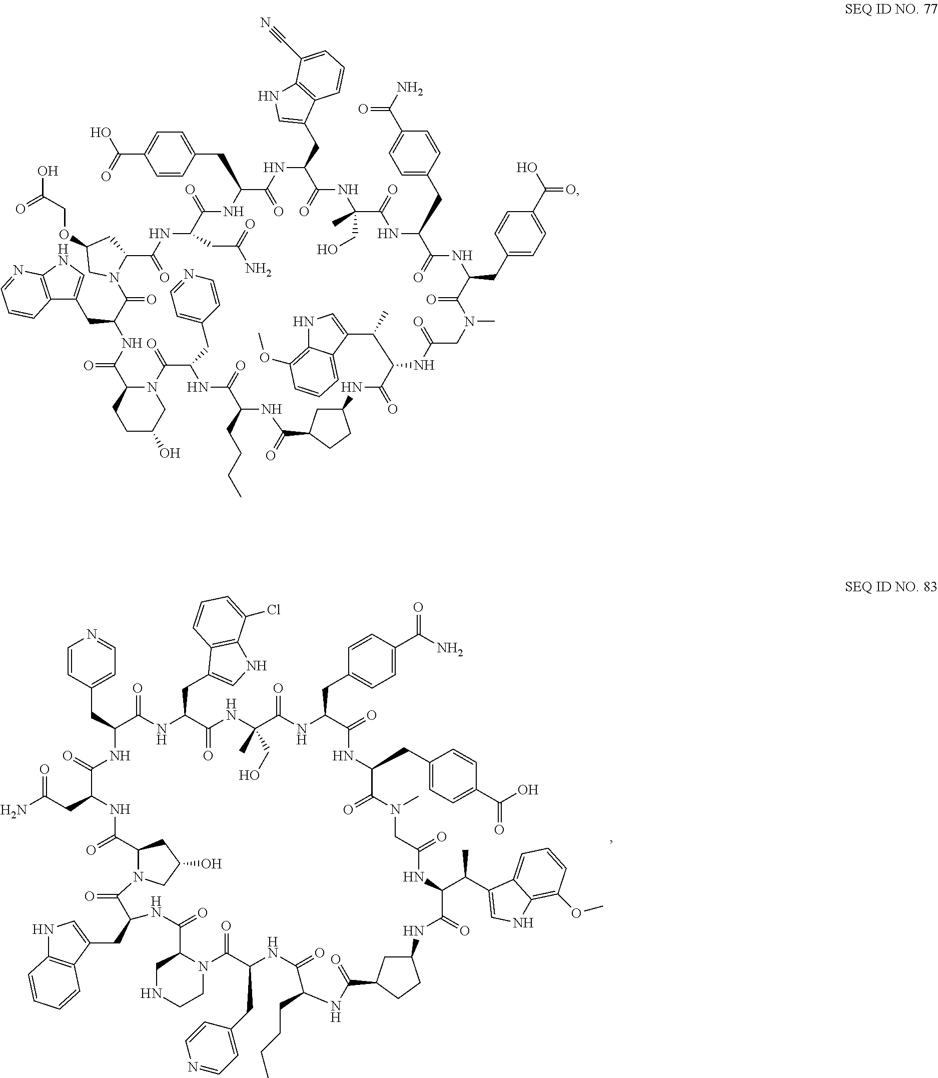 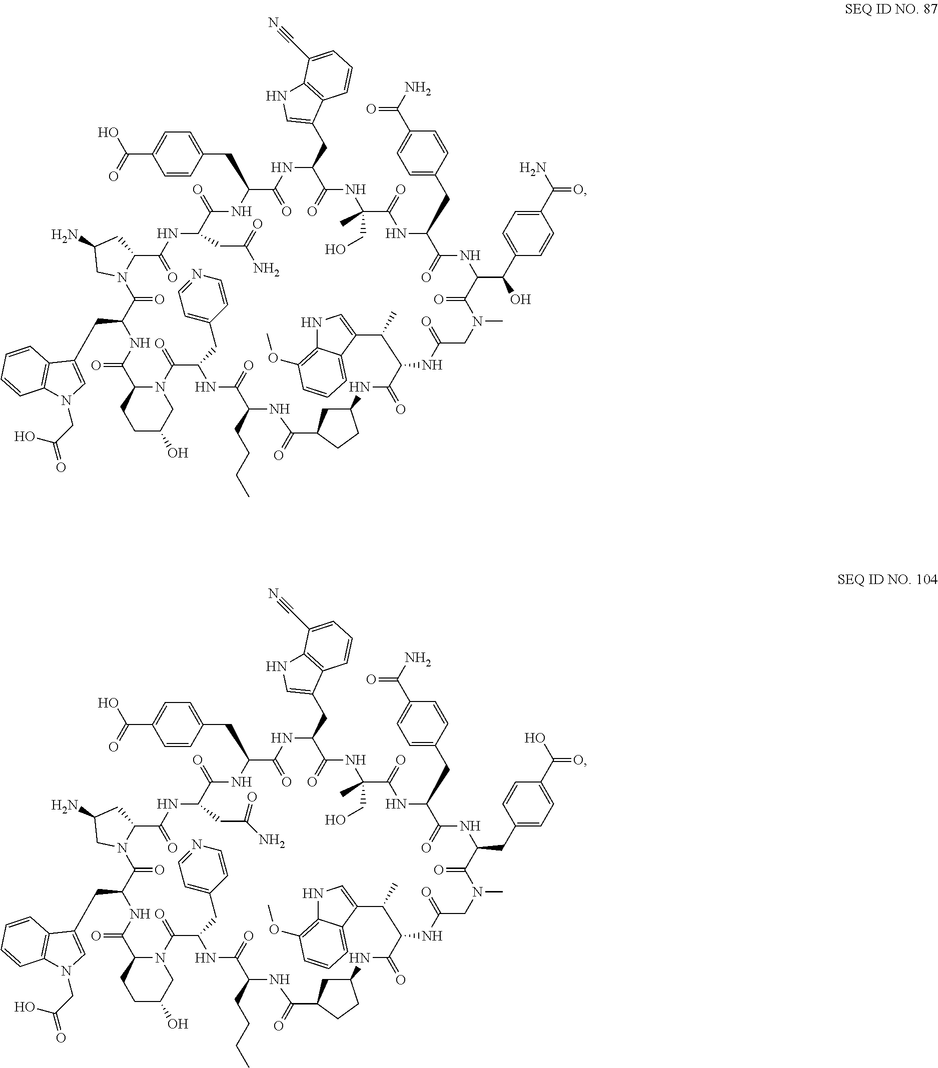 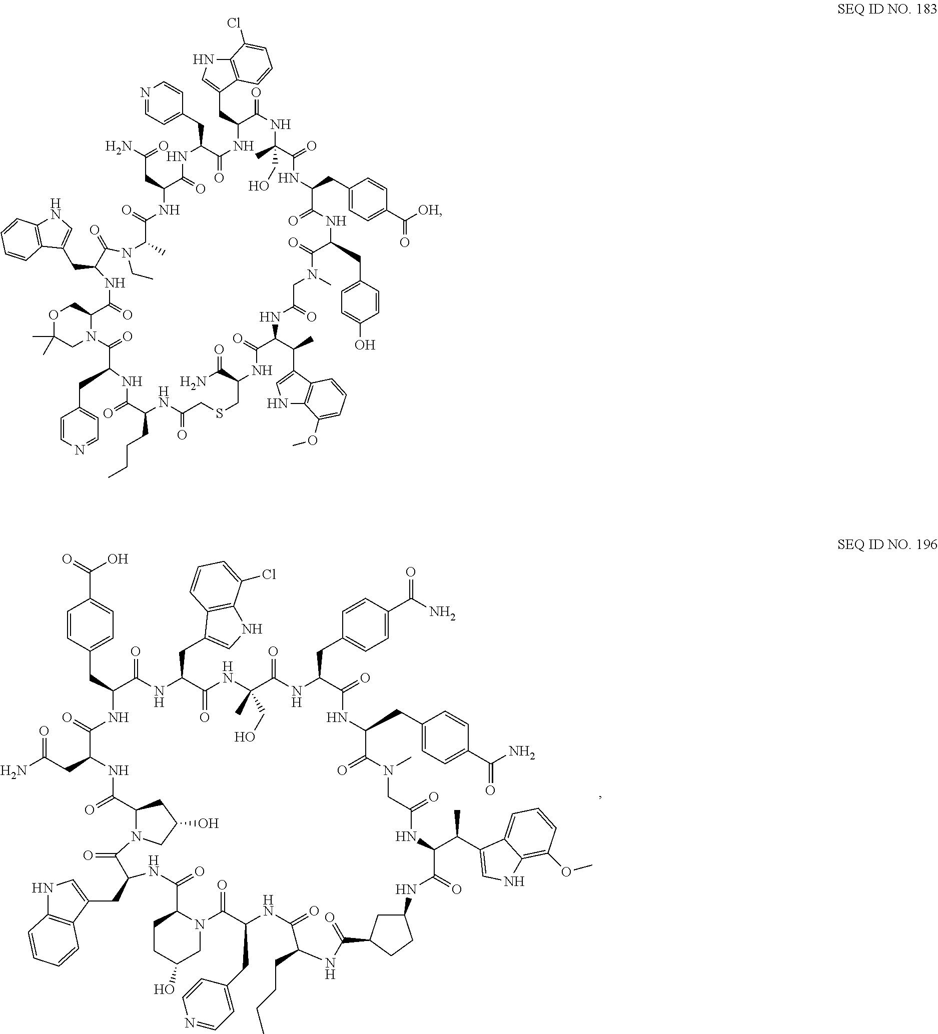 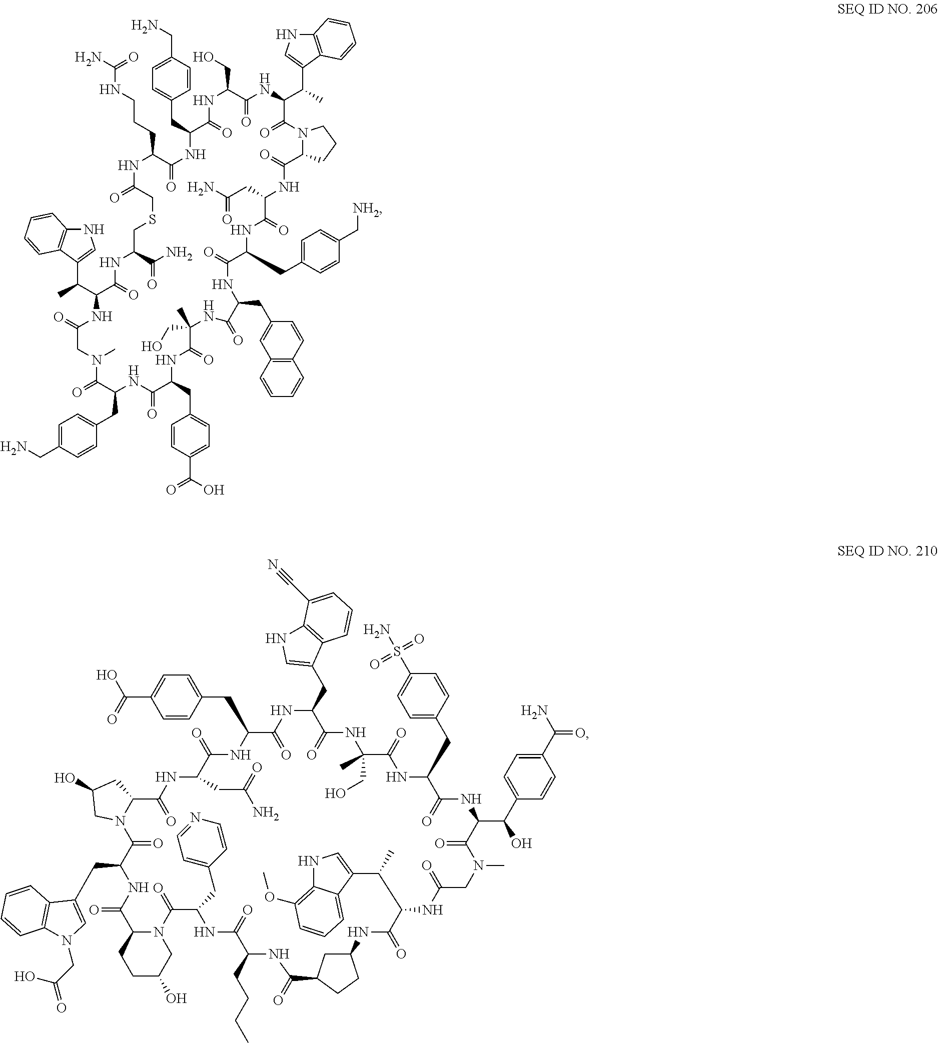 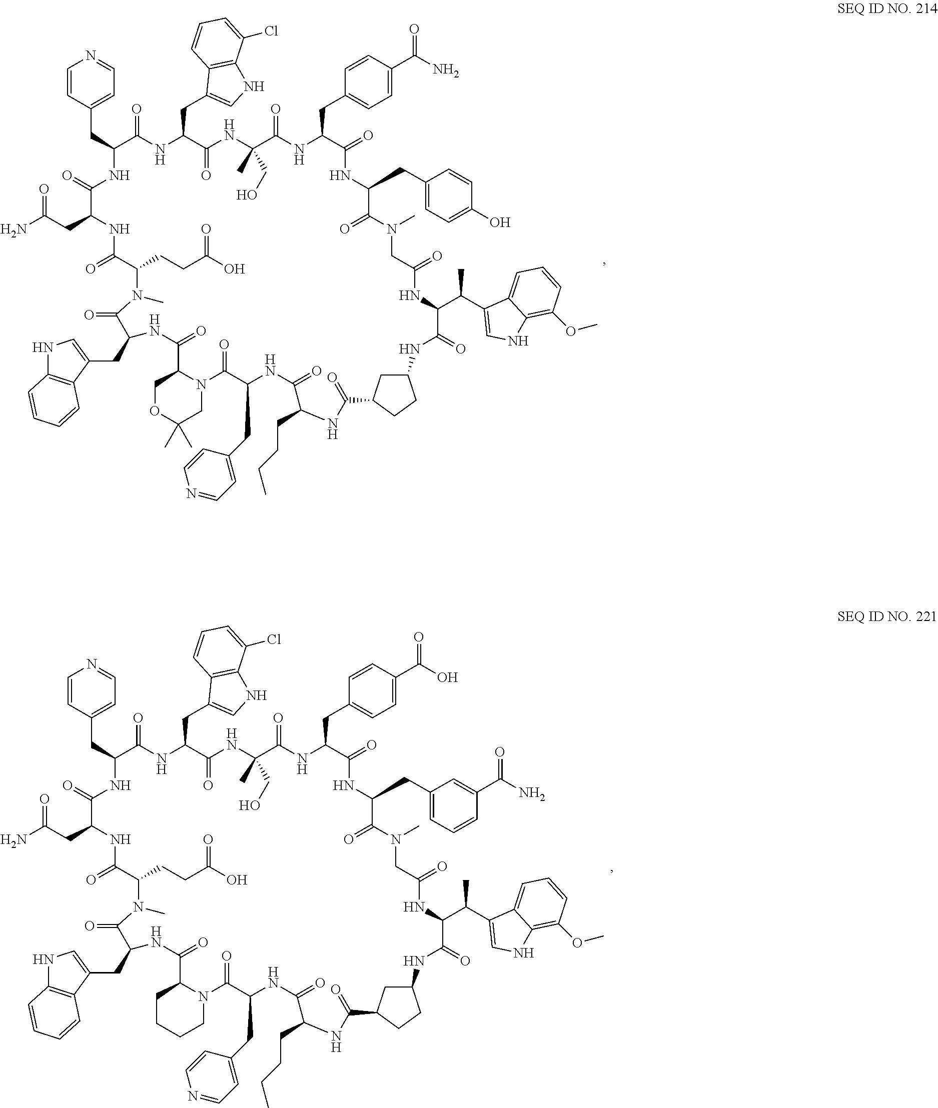 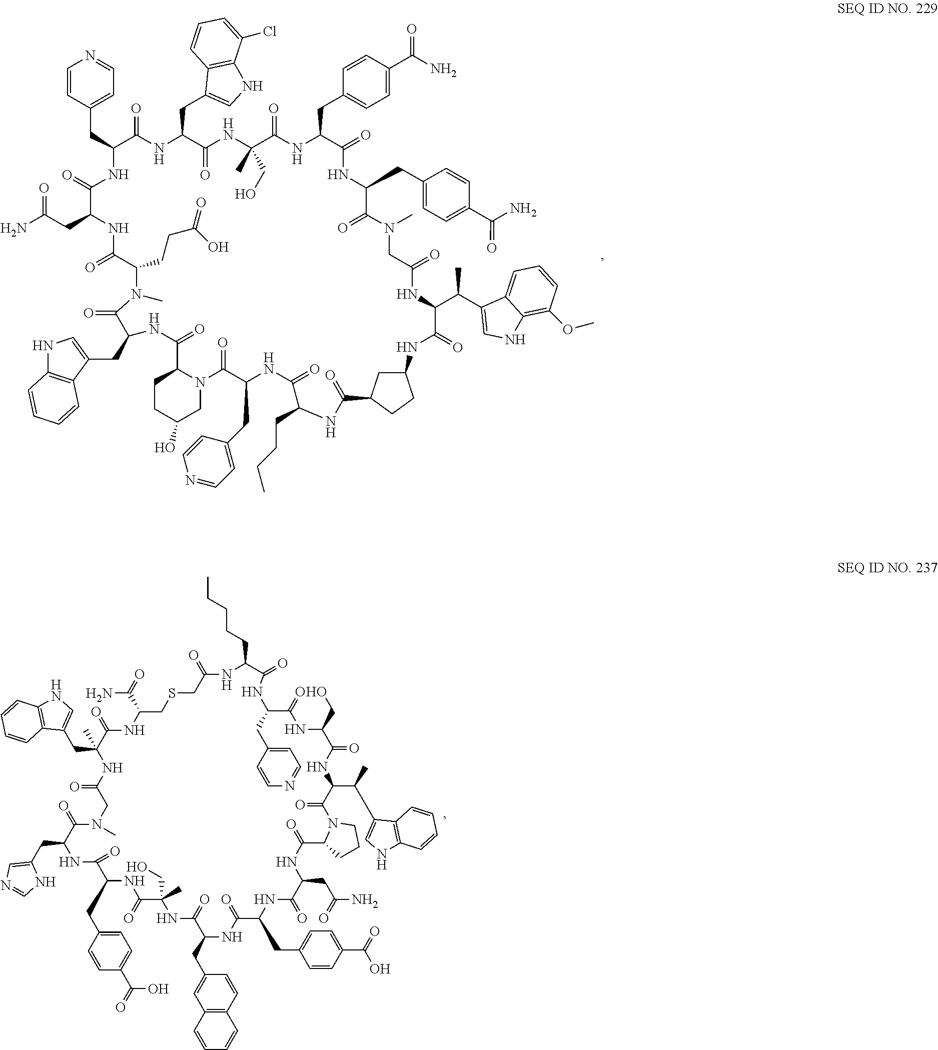 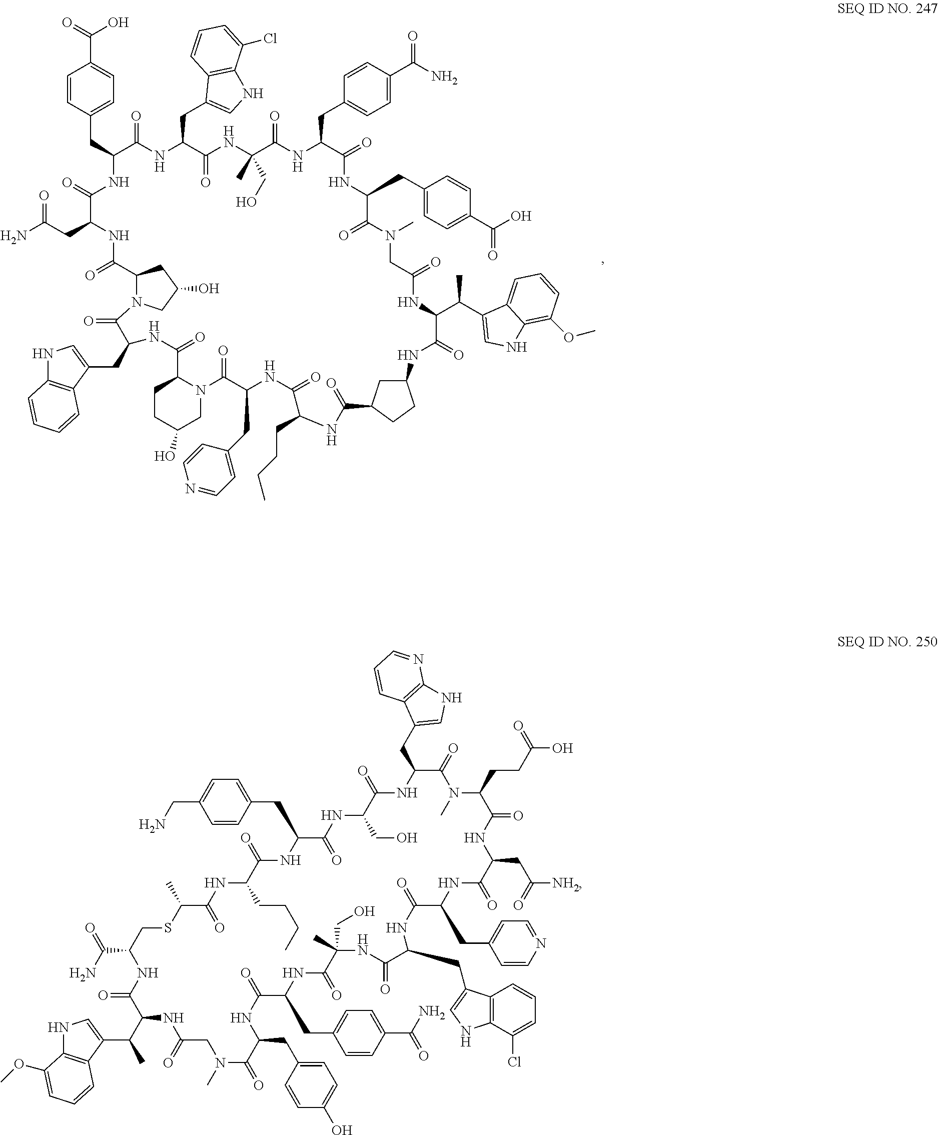 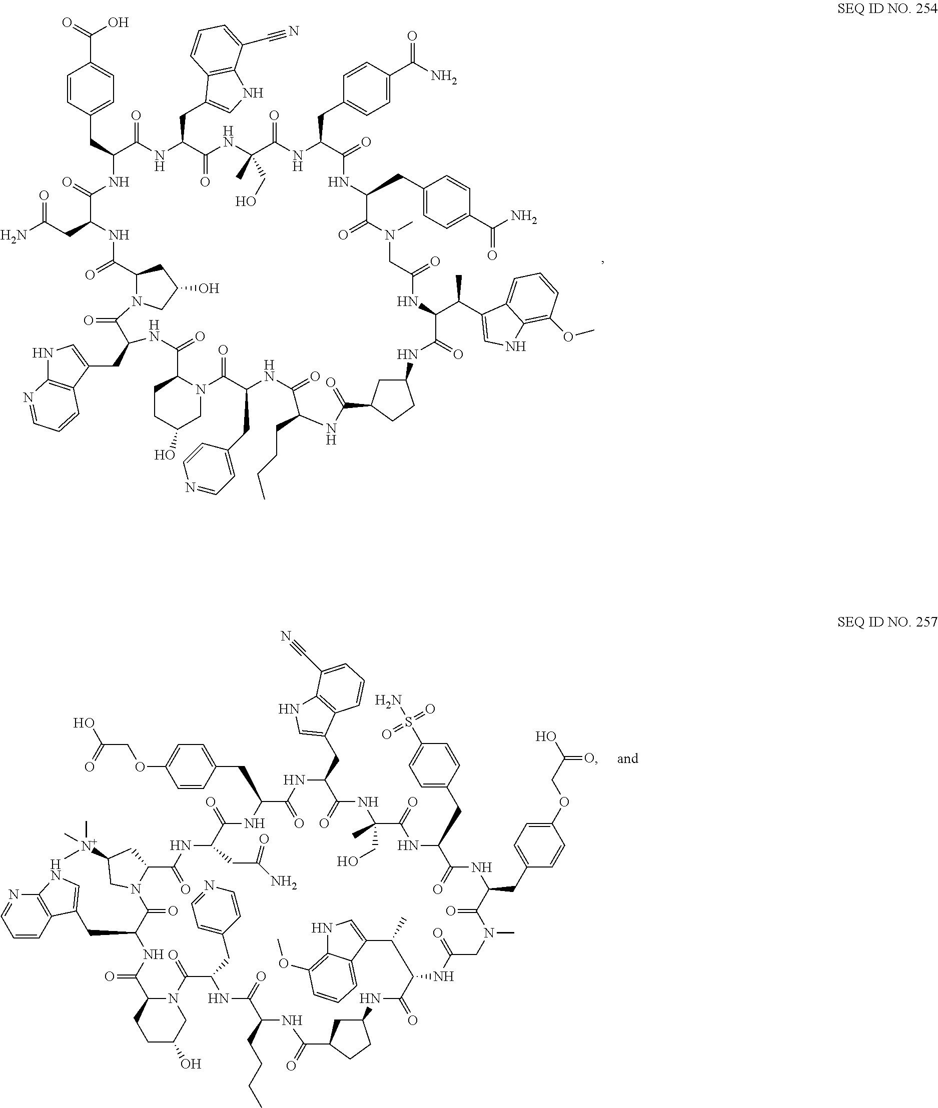 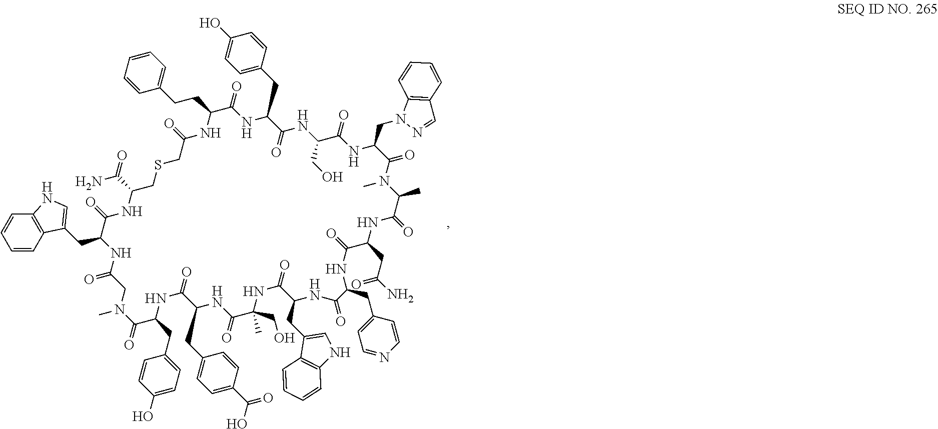 [SMILES: C[C@H]1C(=O)N[C@@H](CC(N)=O)C(=O)N[C@@H](Cc2ccncc2)C(=O)N[C@@H](Cc2c[nH]c3ccccc23)C(=O)N[C@@](C)(CO)C(=O)N[C@@H](Cc2ccc(C(=O)O)cc2)C(=O)N[C@@H](Cc2ccc(O)cc2)C(=O)N(C)CC(=O)N[C@@H](Cc2c[nH]c3ccccc23)C(=O)N[C@H](C(N)=O)CSCC(=O)N[C@@H](CCc2ccccc2)C(=O)N[C@@H](Cc2ccc(O)cc2)C(=O)N[C@@H](CO)C(=O)N[C@@H](Cn2ncc3ccccc32)C(=O)N1C]
or a pharmaceutically acceptable salt thereof.

14. A pharmaceutical composition comprising the compound of claim 1 or a pharmaceutically acceptable salt thereof, and a pharmaceutically acceptable carrier.

15. A method of treating IBD and other TNFα-driven inflammatory diseases comprising administering a therapeutically effective amount of a compound of claim 1 or a pharmaceutically acceptable salt thereof, to a subject in need of such treatment, wherein said IBD and other TNFα-driven inflammatory diseases are selected from ulcerative colitis, Crohn's disease, rheumatoid arthritis, juvenile rheumatoid arthritis, psoriasis, psoriatic arthritis, ankylosing spondylitis, non-radiographic axial spondyloarthritis, and hidradenitis suppurativa.

16. The method according to claim 15, wherein said IBD and other TNFα-driven inflammatory diseases are selected from ulcerative colitis and Crohn's disease.

17. The method according to claim 15, wherein said IBD and other TNFα-driven inflammatory diseases are selected from rheumatoid arthritis, juvenile rheumatoid arthritis, psoriasis, psoriatic arthritis, ankylosing spondylitis, non-radiographic axial spondyloarthritis, and hidradenitis suppurativa.

18. The method of claim 15 wherein the effective amount of the compound or a pharmaceutically acceptable salt thereof is administered orally to the subject.


SEQUENCE LISTING (301 sequences)

SEQ ID NO: 1 - protein, 17 aa, synthetic construct
  SITE 1: note = Acetylated residue
  SITE 5: note = Sarcosine
  SITE 12: note = Sarcosine
  SITE 16: note = 2-(2-Aminoethoxy)acetic acid
  SITE 17: note = Lysine modified with biotin
  CROSSLNK 1..14: note = Position 1 connected to position 14 via an Acetyl cross-linker
FYSWGNYWSYYGWCGXK

SEQ ID NO: 2 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 5: note = (S)-2-Amino-3-[1-(carboxymethyl)indol-3-yl]propanoi c acid
  SITE 6: note = (2R,4S)-4-(Dimethylamino)pyrrolidine-2-carboxylic acid
  SITE 8: note = l-Phenylalanine-4-carboxylic acid
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-sulfamoylphenyl)propanoic acid
  SITE 12: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
XXXXXXNFXSXXGX

SEQ ID NO: 3 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 6: note = (2R,4S)-4-Hydroxypyrrolidine-2-carboxylic acid
  SITE 8: note = (S)-2-Amino-3-(4-(carboxymethoxy)phenyl)propanoic acid
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = (S)-2-Amino-3-(4-(carboxymethoxy)phenyl)propanoic acid
  SITE 13: note = Sarcosine
  SITE 14: note = (2S,3S)-2-Amino-3-(7-methoxy-1H-indol-3-yl)butanoic acid
XXXXWXNXXSXXGX

SEQ ID NO: 4 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 5: note = (S)-2-Amino-3-(1H-pyrrolo[2,3-b]pyridin-3-yl)propan oic acid
  SITE 6: note = (2R,4S)-4-(Carboxymethoxy)pyrrolidine-2-carboxylic acid
  SITE 8: note = l-Phenylalanine-4-carboxylic acid
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-sulfamoylphenyl)propanoic acid
  SITE 12: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
XXXXXXNFXSXFGX

SEQ ID NO: 5 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 6 - skipped

SEQ ID NO: 7 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = 4-[(S)-2-Amino-2-carboxyethyl]-3-fluorobenzoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 8 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 5: note = (S)-2-Amino-3-(1H-pyrrolo[2,3-b]pyridin-3-yl)propan oic acid
  SITE 6: note = (2R,4S)-4-Hydroxypyrrolidine-2-carboxylic acid
  SITE 8: note = (S)-4-(2-Amino-2-carboxyethyl)-2-fluorobenzoic acid
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
XXXXXXNXXSXFGX

SEQ ID NO: 9 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 5: note = (S)-2-Amino-3-[1-(carboxymethyl)indol-3-yl]propanoi c acid
  SITE 6: note = (3S,5R)-5-Carboxy-N,N,N-trimethylpyrrolidin-3-amini um
  SITE 8: note = l-Phenylalanine-4-carboxylic acid
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-sulfamoylphenyl)propanoic acid
  SITE 12: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
XXXXXXNFXSXXGX

SEQ ID NO: 10 - skipped

SEQ ID NO: 11 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 6: note = (2R,4S)-4-(2H-Tetrazol-5-yl)pyrrolidine-2-carboxyli c acid
  SITE 8: note = l-Phenylalanine-4-carboxylic acid
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
XXXXWXNFXSXFGX

SEQ ID NO: 12 - protein, 14 aa, synthetic construct
  SITE 1: note = (1S,2R,3S,4R)-4-Amino-2,3-dihydroxycyclopentane-1-c arboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 5: note = (S)-2-Amino-3-(1H-pyrrolo[2,3-b]pyridin-3-yl)propan oic acid
  SITE 6: note = (2R,4S)-4-Hydroxypyrrolidine-2-carboxylic acid
  SITE 8: note = l-Phenylalanine-4-carboxylic acid
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
XXXXXXNFXSXFGX

SEQ ID NO: 13 - protein, 14 aa, synthetic construct
  SITE 1: note = Acetyl Norleucine
  SITE 2: note = 4-Pyridylalanine
  SITE 3: note = (S)-Piperidine-2-carboxylic acid
  SITE 5: note = 2-[(1-Hydroxycyclopropyl)methylamino]acetic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXWXNXXXXYGXC

SEQ ID NO: 14 - skipped

SEQ ID NO: 15 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 6: note = (2R,4R)-4-(Aminomethyl)pyrrolidine-2-carboxylic acid
  SITE 8: note = l-Phenylalanine-4-carboxylic acid
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
XXXXWXNFXSXFGX

SEQ ID NO: 16 - protein, 14 aa, synthetic construct
  SITE 1: note = (1R,3S)-3-aminocyclopentane-1-carboxylic acid
  SITE 2: note = (S)-2-aminoheptanoic acid
  SITE 3: note = (2S,3R)-2-Amino-3-hydroxy-3-pyridin-4-ylpropanoic acid
  SITE 6: note = (2R,4S)-4-Hydroxypyrrolidine-2-carboxylic acid
  SITE 8: note = l-Phenylalanine-4-carboxylic acid
  SITE 9: note = (S)-2-amino-3-(7-cyanoindolyl)propionic acid
  SITE 10: note = alpha-Methyl-l-serine
  SITE 11: note = (S)-2-Amino-3-(4-carbamoylphenyl)propanoic acid
  SITE 12: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXSWXNXXXXXGX

SEQ ID NO: 17-18 - skipped (2 entries)

SEQ ID NO: 19 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 6: note = N-Methyl-l-glutamic acid
  SITE 8: note = l-Phenylalanine-4-carboxylic acid
  SITE 9: note = (S)-2-amino-3-(7-cyanoindolyl)propionic acid
  SITE 10: note = alpha-Methyl-l-serine
  SITE 11: note = (S)-2-Amino-3-(4-carbamoylphenyl)propanoic acid
  SITE 12: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXXGX

SEQ ID NO: 20 - protein, 14 aa, synthetic construct
  SITE 1: note = (1S,2R,3S,4R)-4-amino-2,3-dihydroxycyclopentane-1-c arboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1S,2R,3S,4 R)-4-amino-2,3-dihydroxycyclopentane-1-carboxylic acid
XXXXWENXXXXYGX

SEQ ID NO: 21-22 - skipped (2 entries)

SEQ ID NO: 23 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 5: note = (S)-2-Amino-3-(1H-pyrrolo[2,3-b]pyridin-3-yl)propan oic acid
  SITE 6: note = (S)-(1-Hydroxypropan-2-yl)glycine
  SITE 8: note = l-Phenylalanine-4-carboxylic acid
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
XXXXXXNFXSXXGX

SEQ ID NO: 24 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 12: note = (S)-2-Amino-3-(4-hydroxy-3-methoxyphenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXXGX

SEQ ID NO: 25 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 6: note = (2R,4S)-4-Hydroxypyrrolidine-2-carboxylic acid
  SITE 7: note = (2S,3S)-2,4-Diamino-3-hydroxy-4-oxobutanoic acid
  SITE 8: note = 4-Pyridylalanine
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
XXXXWXXXXSXFGX

SEQ ID NO: 26 - skipped

SEQ ID NO: 27 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 6: note = (2R,4S)-4-Hydroxypyrrolidine-2-carboxylic acid
  SITE 8: note = l-Phenylalanine-4-carboxylic acid
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = l-Phenylalanine-4-carboxylic acid
  SITE 13: note = Sarcosine
  SITE 14: note = (2S,3S)-2-Amino-3-(1H-indol-3-yl)hexanoic acid
XXXXWXNFXSXFGX

SEQ ID NO: 28 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = (S)-3-(2-Amino-2-carboxyethyl)benzoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 29 - protein, 14 aa, synthetic construct
  SITE 1: note = Acetyl Norleucine
  SITE 2: note = 4-Pyridylalanine
  SITE 3: note = N-Methyl-l-threonine
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXTXENXXXXYGXC

SEQ ID NO: 30 - skipped

SEQ ID NO: 31 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 5: note = (S)-2-Amino-3-(1H-pyrrolo[2,3-b]pyridin-3-yl)propan oic acid
  SITE 6: note = (2R,4S)-4-(Carboxymethoxy)pyrrolidine-2-carboxylic acid
  SITE 8: note = 4-Pyridylalanine
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-sulfamoylphenyl)propanoic acid
  SITE 12: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
XXXXXXNXXSXFGX

SEQ ID NO: 32 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 5: note = (S)-2-Amino-3-[1-(carboxymethyl)pyrrolo[2,3-b]pyrid in-3-yl]propanoic acid
  SITE 6: note = (2R,4S)-4-(Dimethylamino)pyrrolidine-2-carboxylic acid
  SITE 8: note = l-Phenylalanine-4-carboxylic acid
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
XXXXXXNFXSXXGX

SEQ ID NO: 33 - protein, 14 aa, synthetic construct
  SITE 1: note = Acetyl Norleucine
  SITE 2: note = (S)-2-amino-3-(1-bicyclo[1.1.1]pentanyl)propanoic acid
  SITE 3: note = (S)-Piperazine-2-carboxylic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXXENXXXXYGXC

SEQ ID NO: 34 - protein, 14 aa, synthetic construct
  SITE 1: note = (1S,3R,4R)-3-amino-4-hydroxycyclopentane-1-carboxyl ic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1S,3R,4R)-3-amino-4-hydroxycyclopentane-1-carboxylic acid
XXXXWENXXXXYGX

SEQ ID NO: 35 - protein, 14 aa, synthetic construct
  SITE 1: note = (1R,3S)-3-aminocyclopentane-1-carboxylic acid
  SITE 2: note = l-Homoarginine
  SITE 3: note = 4-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 36 - protein, 14 aa, synthetic construct
  SITE 1: note = Acetylated residue
  SITE 4: note = (2S,3S)-2-Amino-3-(1H-indol-3-yl)butanoic acid
  SITE 5: note = N-Methyl-l-alanine
  SITE 7: note = 4-Pyridylalanine
  SITE 9: note = alpha-Methyl-l-serine
  SITE 10: note = l-Phenylalanine-4-carboxylic acid
  SITE 12: note = Sarcosine
  SITE 14: note = Amidated residue
  CROSSLNK 1..14: note = Position 1 connected to position 14 via an Acetyl cross-linker
KYSXANXWXXYGWC

SEQ ID NO: 37 - protein, 14 aa, synthetic construct
  SITE 1: note = (1R,3S)-3-Aminocyclopentane-1-carboxylic acid
  SITE 2: note = l-Norleucine
  SITE 3: note = (S)-2-Amino-3-(pyrimidin-5-yl)propanoic acid
  SITE 4: note = (2S,5R)-5-Hydroxypiperidine-2-carboxylic acid
  SITE 6: note = (2R,4S)-4-Aminopyrrolidine-2-carboxylic acid
  SITE 8: note = (S)-2-Amino-3-(4-(carboxymethoxy)phenyl)propanoic acid
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-sulfamoylphenyl)propanoic acid
  SITE 12: note = (S)-2-Amino-3-(4-(carboxymethoxy)phenyl)propanoic acid
  SITE 13: note = Sarcosine
  SITE 14: note = (2S,3S)-2-Amino-3-(7-methoxy-1H-indol-3-yl)butanoic acid
XXXXWXNXXSXXGX

SEQ ID NO: 38 - protein, 14 aa, synthetic construct
  SITE 1: note = Acetyl Norleucine
  SITE 2: note = 4-Pyridylalanine
  SITE 3: note = N-Ethyl-l-alanine
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXAXENXXXXYGXC

SEQ ID NO: 39 - protein, 14 aa, synthetic construct
  SITE 1: note = Acetyl Norleucine
  SITE 2: note = 4-Pyridylalanine
  SITE 3: note = (2S,5R)-5-Hydroxypiperidine-2-carboxylic acid
  SITE 5: note = N-Methyl-l-glutamic acid
  SITE 7: note = 4-Pyridylalanine
  SITE 8: note = (S)-2-Amino-3-(7-bromo-1H-indol-3-yl)propanoic acid
  SITE 9: note = alpha-Methyl-l-serine
  SITE 10: note = (S)-2-Amino-3-(4-carbamoylphenyl)propanoic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXWENXXXXYGXC

SEQ ID NO: 40 - protein, 14 aa, synthetic construct
  SITE 1: note = (1R,3S)-3-aminocyclopentane-1-carboxylic acid
  SITE 2: note = l-Norleucine
  SITE 3: note = 3-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 41 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 6: note = (2R,4S)-4-Hydroxypyrrolidine-2-carboxylic acid
  SITE 8: note = (S)-4-(2-Amino-2-carboxyethyl)-3-fluorobenzoic acid
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = (S)-4-(2-Amino-2-carboxyethyl)-3-fluorobenzoic acid
  SITE 13: note = Sarcosine
  SITE 14: note = (2S,3S)-2-Amino-3-(7-methoxy-1H-indol-3-yl)butanoic acid
XXXXWXNXXSXXGX

SEQ ID NO: 42 - protein, 14 aa, synthetic construct
  SITE 1: note = Acetyl Norleucine
  SITE 2: note = 4-Pyridylalanine
  SITE 3: note = (S)-Piperidine-2-carboxylic acid
  SITE 4: note = (S)-2-Amino-3-(1H-pyrrolo[2,3-b]pyridin-3-yl)propan oic acid
  SITE 5: note = 2-(3-Methoxypropylamino)acetic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXXXNXXXXYGXC

SEQ ID NO: 43 - skipped

SEQ ID NO: 44 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 6: note = N-Methyl-l-glutamic acid
  SITE 8: note = (S)-2-Amino-3-(4-(carboxymethoxy)phenyl)propanoic acid
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 45 - skipped

SEQ ID NO: 46 - protein, 14 aa, synthetic construct
  SITE 1: note = Acetyl Norleucine
  SITE 2: note = (2S,3S)-2-Amino-3-(4-hydroxyphenyl)butanoic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXSXENXXXXYGXC

SEQ ID NO: 47 - protein, 14 aa, synthetic construct
  SITE 1: note = (1R,3S)-3-aminocyclopentane-1-carboxylic acid
  SITE 2: note = [(S)-5-Amino-5-carboxypentyl]-trimethylazanium
  SITE 3: note = 4-Pyridylalanine
  SITE 4: note = (S)-Piperidine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 48-51 - skipped (4 entries)

SEQ ID NO: 52 - protein, 14 aa, synthetic construct
  SITE 1: note = Acetyl Norleucine
  SITE 2: note = 4-Pyridylalanine
  SITE 3: note = (S)-Piperazine-2-carboxylic acid
  SITE 4: note = (S)-2-Amino-3-[1-(carboxymethyl)indol-3-yl]propanoi c acid
  SITE 5: note = N-Methyl-l-glutamic acid
  SITE 7: note = l-Phenylalanine-4-carboxylic acid
  SITE 8: note = (S)-2-Amino-3-(7-chloro-1H-indol-3-yl)propanoic acid
  SITE 9: note = alpha-Methyl-l-serine
  SITE 10: note = (S)-2-Amino-3-(4-carbamoylphenyl)propanoic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXXENXXXXYGXC

SEQ ID NO: 53-54 - skipped (2 entries)

SEQ ID NO: 55 - protein, 14 aa, synthetic construct
  SITE 1: note = (1R,3S)-3-aminocyclopentane-1-carboxylic acid
  SITE 2: note = (S)-2-aminoheptanoic acid
  SITE 3: note = (2S,3S)-2-Amino-3-pyridin-4-ylbutanoic acid
  SITE 6: note = (2R,4S)-4-Hydroxypyrrolidine-2-carboxylic acid
  SITE 8: note = l-Phenylalanine-4-carboxylic acid
  SITE 9: note = (S)-2-amino-3-(7-cyanoindolyl)propionic acid
  SITE 10: note = alpha-Methyl-l-serine
  SITE 11: note = (S)-2-Amino-3-(4-carbamoylphenyl)propanoic acid
  SITE 12: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXDWXNXXXXXGX

SEQ ID NO: 56 - protein, 14 aa, synthetic construct
  SITE 1: note = Acetyl Norleucine
  SITE 2: note = 4-Pyridylalanine
  SITE 3: note = l-Cyclobutylglycine
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = alpha-Methyl-l-tryptophan
  SITE 14: note = Amidated residue
  CROSSLNK 1..14: note = Position 1 connected to position 14 via an Acetyl cross-linker
XXXXENXXXXYGXC

SEQ ID NO: 57 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 5: note = (S)-2-Amino-3-(1H-pyrrolo[2,3-b]pyridin-3-yl)propan oic acid
  SITE 6: note = (2R,4S)-4-Hydroxypyrrolidine-2-carboxylic acid
  SITE 8: note = (S)-5-(2-Amino-2-carboxyethyl)picolinic acid
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
XXXXXXNXXSXFGX

SEQ ID NO: 58 - protein, 14 aa, synthetic construct
  SITE 1: note = Acetyl Norleucine
  SITE 2: note = 4-Pyridylalanine
  SITE 3: note = (S)-Piperidine-2-carboxylic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = (S)-2-Amino-3-(4-(aminomethyl)phenyl)propanoic acid
  SITE 8: note = (S)-2-Amino-3-(7-chloro-1H-indol-3-yl)propanoic acid
  SITE 9: note = alpha-Methyl-l-serine
  SITE 10: note = (S)-2-Amino-3-(4-(aminomethyl)phenyl)propanoic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXXENXXXXYGXC

SEQ ID NO: 59 - protein, 14 aa, synthetic construct
  SITE 1: note = Acetyl Norleucine
  SITE 2: note = (S)-2-Amino-3-(4-(aminomethyl)phenyl)propanoic acid
  SITE 3: note = N-Methyl-l-serine
  SITE 4: note = (S)-2-Amino-3-(1H-pyrrolo[2,3-b]pyridin-3-yl)propan oic acid
  SITE 5: note = N-Ethyl-l-alanine
  SITE 7: note = l-Phenylalanine-4-carboxylic acid
  SITE 8: note = (S)-2-Amino-3-(7-chloro-1H-indol-3-yl)propanoic acid
  SITE 9: note = alpha-Methyl-l-serine
  SITE 10: note = (S)-2-Amino-3-(4-carbamoylphenyl)propanoic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXSXANXXXXYGXC

SEQ ID NO: 60 - protein, 14 aa, synthetic construct
  SITE 1: note = (S)-2-Ethylacetyl Norleucine
  SITE 2: note = 4-Pyridylalanine
  SITE 3: note = (S)-6,6-dimethylmorpholine-3-carboxylic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via a (S)-2-Ethylacetyl cross-linker
XXXWENXXXXYGXC

SEQ ID NO: 61 - protein, 14 aa, synthetic construct
  SITE 1: note = Acetyl Norleucine
  SITE 2: note = 4-Pyridylalanine
  SITE 3: note = (S)-Piperidine-2-carboxylic acid
  SITE 4: note = (S)-2-Amino-3-(1H-pyrrolo[2,3-b]pyridin-3-yl)propan oic acid
  SITE 5: note = 2-[(3-Hydroxy-2,2-dimethylpropyl)amino]acetic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXXXNXXXXYGXC

SEQ ID NO: 62 - protein, 14 aa, synthetic construct
  SITE 1: note = Acetyl Norleucine
  SITE 2: note = 4-Pyridylalanine
  SITE 3: note = (S)-Piperidine-2-carboxylic acid
  SITE 4: note = (S)-2-Amino-3-(1H-pyrrolo[2,3-b]pyridin-3-yl)propan oic acid
  SITE 5: note = 2-(2-Methoxyethylamino)acetic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXXXNXXXXYGXC

SEQ ID NO: 63 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 12: note = (S)-3-[4-(4-Acetylpiperazin-1-yl)phenyl]-2-aminopro panoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXXGX

SEQ ID NO: 64 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 7: note = l-Methionine sulfone
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWEXXXXXYGX

SEQ ID NO: 65 - protein, 14 aa, synthetic construct
  SITE 1: note = Acetyl Norleucine
  SITE 2: note = (S)-2-Amino-3-(pyrimidin-5-yl)propanoic acid
  SITE 3: note = (S)-Piperazine-2-carboxylic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXXENXXXXYGXC

SEQ ID NO: 66 - protein, 14 aa, synthetic construct
  SITE 1: note = Acetyl Norleucine
  SITE 2: note = (S)-2-amino-3-(3-hydroxy-1-bicyclo[1.1.1]pentanyl)p ropanoic acid
  SITE 3: note = l-Homoserine
  SITE 4: note = 1-Methyl- l-tryptophan
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXXENXXXXYGXC

SEQ ID NO: 67 - skipped

SEQ ID NO: 68 - protein, 14 aa, synthetic construct
  SITE 1: note = Acetyl Norleucine
  SITE 2: note = 4-Pyridylalanine
  SITE 3: note = (S)-Piperazine-2-carboxylic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXXENXXXXYGXC

SEQ ID NO: 69 - protein, 14 aa, synthetic construct
  SITE 1: note = cis-3-Aminocyclobutane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via cis-3-Aminocyclobutane-1-carboxylic acid
XXXXWENXXXXYGX

SEQ ID NO: 70 - protein, 14 aa, synthetic construct
  SITE 1: note = (1R,3S)-3-aminocyclopentane-1-carboxylic acid
  SITE 2: note = (S)-2-aminoheptanoic acid
  SITE 3: note = (2S,3S)-2-Amino-3-pyridin-4-ylbutanoic acid
  SITE 6: note = (2R,4S)-4-Hydroxypyrrolidine-2-carboxylic acid
  SITE 8: note = l-Phenylalanine-4-carboxylic acid
  SITE 9: note = (S)-2-amino-3-(7-cyanoindolyl)propionic acid
  SITE 10: note = alpha-Methyl-l-serine
  SITE 11: note = (S)-2-Amino-3-(4-carbamoylphenyl)propanoic acid
  SITE 12: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXQWXNXXXXXGX

SEQ ID NO: 71 - protein, 14 aa, synthetic construct
  SITE 1: note = Acetyl Norleucine
  SITE 2: note = 4-Pyridylalanine
  SITE 3: note = (2S,4R)-4-Hydroxypiperidine-2-carboxylic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXWENXXXXYGXC

SEQ ID NO: 72 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = 2-(2-Hydroxyethylamino)acetic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWXNXXXXYGX

SEQ ID NO: 73 - protein, 14 aa, synthetic construct
  SITE 1: note = (1S,4R)-4-aminocyclopent-2-ene-1-carboxylic acid
  SITE 2: note = l-Homophenylalanine
  SITE 3: note = 4-Pyridylalanine
  SITE 6: note = N-Methyl-l-alanine
  SITE 8: note = 4-Pyridylalanine
  SITE 10: note = alpha-Methyl-l-serine
  SITE 11: note = l-Phenylalanine-4-carboxylic acid
  SITE 13: note = Sarcosine
  SITE 1..14: note = Position 1 connected to position 14 via (1S,4R)-4-aminocyclopent-2-ene-1-carboxylic acid
XXXSWANXWXXYGW

SEQ ID NO: 74 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 5: note = (S)-2-amino-3-(1H-indazol-1-yl)propanoic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXXENXXXXYGX

SEQ ID NO: 75 - protein, 14 aa, synthetic construct
  SITE 1: note = Acetyl Norleucine
  SITE 2: note = 4-Pyridylalanine
  SITE 3: note = (S)-Piperazine-2-carboxylic acid
  SITE 5: note = N-Methyl-l-glutamic acid
  SITE 6: note = N5,N5-Dimethyl- l-glutamine
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXWEQXXXXYGXC

SEQ ID NO: 76 - protein, 14 aa, synthetic construct
  SITE 1: note = Acetyl Norleucine
  SITE 2: note = 4-Pyridylalanine
  SITE 3: note = (S)-Piperazine-2-carboxylic acid
  SITE 5: note = alpha-methyl-d-proline
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXWXNXXXXYGXC

SEQ ID NO: 77 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 5: note = (S)-2-Amino-3-(1H-pyrrolo[2,3-b]pyridin-3-yl)propan oic acid
  SITE 6: note = (2R,4S)-4-(Carboxymethoxy)pyrrolidine-2-carboxylic acid
  SITE 8: note = l-Phenylalanine-4-carboxylic acid
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
XXXXXXNFXSXXGX

SEQ ID NO: 78 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 6: note = (2R,4S)-4-Hydroxypyrrolidine-2-carboxylic acid
  SITE 7: note = (2S,3R)-2,4-Diamino-3-hydroxy-4-oxobutanoic acid
  SITE 8: note = 4-Pyridylalanine
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
XXXXWXXXXSXFGX

SEQ ID NO: 79 - skipped

SEQ ID NO: 80 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-(carboxymethoxy)phenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 81 - protein, 14 aa, synthetic construct
  SITE 1: note = (1R,3S)-3-aminocyclopentane-1-carboxylic acid
  SITE 2: note = (S)-2-aminononanoic acid
  SITE 3: note = 4-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 82 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = 2-(2-Methoxyethylamino)acetic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWXNXXXXYGX

SEQ ID NO: 83-85 - skipped (3 entries)

SEQ ID NO: 86 - protein, 14 aa, synthetic construct
  SITE 1: note = (1R,3S)-3-aminocyclopentane-1-carboxylic acid
  SITE 2: note = (S)-2-Amino-6-(methylamino)hexanoic acid
  SITE 3: note = 4-Pyridylalanine
  SITE 4: note = (2S,5R)-5-Hydroxypiperidine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 87 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 5: note = (S)-2-Amino-3-[1-(carboxymethyl)indol-3-yl]propanoi c acid
  SITE 6: note = (2R,4S)-4-Aminopyrrolidine-2-carboxylic acid
  SITE 8: note = l-Phenylalanine-4-carboxylic acid
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = (2S,3R)-2-Amino-3-(4-carbamoylphenyl)-3-hydroxyprop anoic acid
  SITE 13: note = Sarcosine
  SITE 14: note = (2S,3S)-2-Amino-3-(7-methoxy-1H-indol-3-yl)butanoic acid
XXXXXXNFXSXXGX

SEQ ID NO: 88 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 6: note = (2R,4S)-4-Hydroxypyrrolidine-2-carboxylic acid
  SITE 8: note = (S)-5-(2-Amino-2-carboxyethyl)pyrimidine-2-carboxyl ic acid
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
XXXXWXNXXSXFGX

SEQ ID NO: 89 - skipped

SEQ ID NO: 90 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 5: note = (S)-2-Amino-3-(1H-pyrrolo[2,3-b]pyridin-3-yl)propan oic acid
  SITE 6: note = (2R,4S)-4-Hydroxypyrrolidine-2-carboxylic acid
  SITE 8: note = l-Phenylalanine-4-carboxylic acid
  SITE 9: note = (S)-2-Amino-3-(7-cyano-1H-indol-3-yl)propanoic acid
  SITE 10: note = alpha-Methyl-l-serine
  SITE 11: note = (2S,3S)-2-Amino-3-(4-carbamoylphenyl)-3-hydroxyprop anoic acid
  SITE 12: note = (2S,3R)-2-Amino-3-(4-carbamoylphenyl)-3-hydroxyprop anoic acid
  SITE 13: note = Sarcosine
  SITE 14: note = (2S,3S)-2-Amino-3-(7-methoxy-1H-indol-3-yl)butanoic acid
XXXXXXNFXSXXGX

SEQ ID NO: 91 - skipped

SEQ ID NO: 92 - protein, 14 aa, synthetic construct
  SITE 1: note = (1R,3S)-3-aminocyclopentane-1-carboxylic acid
  SITE 2: note = (S)-2-aminoheptanoic acid
  SITE 3: note = (2S,3S)-2-Amino-3-pyridin-4-ylbutanoic acid
  SITE 6: note = (2R,4S)-4-Hydroxypyrrolidine-2-carboxylic acid
  SITE 8: note = l-Phenylalanine-4-carboxylic acid
  SITE 9: note = (S)-2-amino-3-(7-cyanoindolyl)propionic acid
  SITE 10: note = alpha-Methyl-l-serine
  SITE 11: note = (S)-2-Amino-3-(4-carbamoylphenyl)propanoic acid
  SITE 12: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXNWXNXXXXXGX

SEQ ID NO: 93 - protein, 14 aa, synthetic construct
  SITE 1: note = Acetyl (S)-2-Amino-6-methylheptanoic acid
  SITE 2: note = 4-Pyridylalanine
  SITE 3: note = (S)-Piperazine-2-carboxylic acid
  SITE 5: note = N-Methyl-l-glutamic acid
  SITE 7: note = l-Phenylalanine-4-carboxylic acid
  SITE 8: note = (S)-2-Amino-3-(7-chloro-1H-indol-3-yl)propanoic acid
  SITE 9: note = alpha-Methyl-l-serine
  SITE 10: note = (S)-2-Amino-3-(4-carbamoylphenyl)propanoic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXWENXXXXYGXC

SEQ ID NO: 94 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = 2-(Ethylamino)acetic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWXNXXXXYGX

SEQ ID NO: 95 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = (2R,4S)-4-(Carboxymethoxy)pyrrolidine-2-carboxylic acid
  SITE 8: note = l-Phenylalanine-4-carboxylic acid
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWXNXXXXYGX

SEQ ID NO: 96 - protein, 14 aa, synthetic construct
  SITE 1: note = Acetyl Norleucine
  SITE 2: note = 4-Pyridylalanine
  SITE 3: note = (2S,5R)-5-Hydroxypiperidine-2-carboxylic acid
  SITE 5: note = N-Methyl-l-glutamic acid
  SITE 7: note = (S)-2-Amino-3-(4-(aminomethyl)phenyl)propanoic acid
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXWENXXXXYGXC

SEQ ID NO: 97 - protein, 14 aa, synthetic construct
  SITE 1: note = Acetyl Norleucine
  SITE 2: note = 4-Pyridylalanine
  SITE 3: note = (2S,5R)-5-Hydroxypiperidine-2-carboxylic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = (S)-2-Amino-3-(4-carbamoylphenyl)propanoic acid
  SITE 12: note = Sarcosine
  SITE 13: note = (2S,3S)-2-Amino-3-(2-methyl-1H-indol-3-yl)butanoic acid
  SITE 14: note = Amidated residue
  CROSSLNK 1..14: note = Position 1 connected to position 14 via an Acetyl cross-linker
XXXWENXXXXYGXC

SEQ ID NO: 98 - protein, 14 aa, synthetic construct
  SITE 1: note = (1R,3S)-3-aminocyclopentane-1-carboxylic acid
  SITE 2: note = (S)-2-aminoheptanoic acid
  SITE 3: note = (2S,3S)-2-Amino-3-pyridin-4-ylbutanoic acid
  SITE 6: note = (2R,4S)-4-Hydroxypyrrolidine-2-carboxylic acid
  SITE 8: note = l-Phenylalanine-4-carboxylic acid
  SITE 9: note = (S)-2-amino-3-(7-cyanoindolyl)propionic acid
  SITE 10: note = alpha-Methyl-l-serine
  SITE 11: note = (S)-2-Amino-3-(4-carbamoylphenyl)propanoic acid
  SITE 12: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXHWXNXXXXXGX

SEQ ID NO: 99 - protein, 14 aa, synthetic construct
  SITE 1: note = Acetyl Norleucine
  SITE 2: note = 4-Pyridylalanine
  SITE 3: note = (S)-4-methoxy-2-(methylamino)butanoic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXXENXXXXYGXC

SEQ ID NO: 100 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = (S)-2-amino-3-(6-methoxypyridin-3-yl)propanoic acid
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 101 - skipped

SEQ ID NO: 102 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 6: note = N-Methyl-l-glutamic acid
  SITE 8: note = (S)-2-Amino-3-(pyrimidin-5-yl)propanoic acid
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 103 - skipped

SEQ ID NO: 104 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 5: note = (S)-2-Amino-3-[1-(carboxymethyl)indol-3-yl]propanoi c acid
  SITE 6: note = (2R,4S)-4-Aminopyrrolidine-2-carboxylic acid
  SITE 8: note = l-Phenylalanine-4-carboxylic acid
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
XXXXXXNFXSXFGX

SEQ ID NO: 105 - protein, 14 aa, synthetic construct
  SITE 1: note = (1R,3S)-3-Aminocyclopentane-1-carboxylic acid
  SITE 2: note = l-Norleucine
  SITE 3: note = (S)-2-Amino-3-(pyridazin-4-yl)propanoic acid
  SITE 4: note = (2S,5R)-5-Hydroxypiperidine-2-carboxylic acid
  SITE 6: note = (2R,4S)-4-Hydroxypyrrolidine-2-carboxylic acid
  SITE 8: note = l-Phenylalanine-4-carboxylic acid
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
XXXXWXNFXSXFGX

SEQ ID NO: 106 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 6: note = (2R,4S)-4-Hydroxypyrrolidine-2-carboxylic acid
  SITE 8: note = l-Phenylalanine-4-carboxylic acid
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = l-Phenylalanine-4-carboxylic acid
  SITE 13: note = Sarcosine
  SITE 14: note = (2S,3S)-2-Amino-3-(7-methoxy-2-methyl-1H-indol-3-yl )butanoic acid
XXXXWXNFXSXFGX

SEQ ID NO: 107 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 12: note = 4-[(S)-2-Amino-2-carboxyethyl]-3-fluorobenzoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXXGX

SEQ ID NO: 108 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = 4-[(S)-2-Amino-2-carboxyethyl]-3-fluorobenzoic acid
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 109-110 - skipped (2 entries)

SEQ ID NO: 111 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-(carboxymethyl)phenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 112 - skipped

SEQ ID NO: 113 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 5: note = (S)-2-Amino-3-(1H-pyrrolo[2,3-b]pyridin-3-yl)propan oic acid
  SITE 6: note = 2-(((3S,5R)-5-Carboxypyrrolidin-3-yl)amino)-N,N, -trimethyl-2-oxoethan-1-aminium
  SITE 8: note = l-Phenylalanine-4-carboxylic acid
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = (S)-4-(2-Amino-2-carboxyethyl)-2-fluorobenzoic acid
  SITE 13: note = Sarcosine
  SITE 14: note = (2S,3S)-2-Amino-3-(7-methoxy-1H-indol-3-yl)butanoic acid
XXXXXXNFXSXXGX

SEQ ID NO: 114-115 - skipped (2 entries)

SEQ ID NO: 116 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 5: note = (S)-2-Amino-3-(1H-pyrrolo[2,3-b]pyridin-3-yl)propan oic acid
  SITE 6: note = 2-(((3S,5R)-5-Carboxypyrrolidin-3-yl)amino)-N,N, -trimethyl-2-oxoethan-1-aminium
  SITE 8: note = (S)-4-(2-Amino-2-carboxyethyl)-2-fluorobenzoic acid
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
XXXXXXNXXSXFGX

SEQ ID NO: 117 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = (S)-2-Amino-3-(4-methoxycarbonylphenyl)propanoic acid
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 118 - protein, 14 aa, synthetic construct
  SITE 1: note = (1R,3S)-3-Aminocyclopentane-1-carboxylic acid
  SITE 2: note = l-Norleucine
  SITE 3: note = 4-((1S,2S)-1-Amino-1-carboxypropan-2-yl)benzoic acid
  SITE 4: note = l-Citrulline
  SITE 5: note = (S)-2-Amino-3-(1H-pyrrolo[2,3-b]pyridin-3-yl)propan oic acid
  SITE 6: note = (2R,4S)-4-Hydroxypyrrolidine-2-carboxylic acid
  SITE 8: note = l-Phenylalanine-4-carboxylic acid
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
XXXXXXNFXSXXGX

SEQ ID NO: 119 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 5: note = (S)-2-Amino-3-[1-(carboxymethyl)indol-3-yl]propanoi c acid
  SITE 6: note = (2R,4S)-4-Hydroxypyrrolidine-2-carboxylic acid
  SITE 8: note = (S)-2-Amino-3-(4-sulfamoylphenyl)propanoic acid
  SITE 9: note = (S)-2-Amino-3-(7-cyano-1H-indol-3-yl)propanoic acid
  SITE 10: note = alpha-Methyl-l-serine
  SITE 11: note = (2S,3R)-2-Amino-3-(4-carbamoylphenyl)-3-hydroxyprop anoic acid
  SITE 12: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
XXXXXXNXXSXFGX

SEQ ID NO: 120 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (2S,5S)-Piperidine-2,5-dicarboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 121 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 5: note = (S)-2-Amino-3-(1H-pyrrolo[2,3-b]pyridin-3-yl)propan oic acid
  SITE 6: note = (2-(1,3,4-Oxadiazol-2-yl)ethyl)glycine
  SITE 8: note = 4-Pyridylalanine
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
XXXXXXNXXSXFGX

SEQ ID NO: 122 - skipped

SEQ ID NO: 123 - protein, 14 aa, synthetic construct
  SITE 1: note = Acetyl Norleucine
  SITE 2: note = (S)-2-Amino-3-(4-carbamoylphenyl)propanoic acid
  SITE 3: note = N-Methyl-l-serine
  SITE 5: note = N-Methyl-l-glutamic acid
  SITE 7: note = l-Phenylalanine-4-carboxylic acid
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXSWENXXXXYGXC

SEQ ID NO: 124 - protein, 14 aa, synthetic construct
  SITE 1: note = Acetyl Norleucine
  SITE 2: note = 4-Pyridylalanine
  SITE 3: note = (S)-Piperazine-2-carboxylic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 11: note = (S)-2-Amino-3-(4-carbamoylphenyl)propanoic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXWENXXXXXGXC

SEQ ID NO: 125 - protein, 14 aa, synthetic construct
  SITE 1: note = Acetyl Norleucine
  SITE 2: note = 4-Pyridylalanine
  SITE 3: note = (2S,4R)-4-Aminopyrrolidine-2-carboxylic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXXENXXXXYGXC

SEQ ID NO: 126 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = (S)-2-Amino-3-(4-(carboxymethoxy)phenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXXGX

SEQ ID NO: 127 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-ureidophenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 128-130 - skipped (3 entries)

SEQ ID NO: 131 - protein, 14 aa, synthetic construct
  SITE 1: note = (1R,3S)-3-aminocyclopentane-1-carboxylic acid
  SITE 2: note = l-Homolysine
  SITE 3: note = 4-Pyridylalanine
  SITE 4: note = (2S,5R)-5-Hydroxypiperidine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 132 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 6: note = (2R,4S)-4-(Carboxymethyl)pyrrolidine-2-carboxylic acid
  SITE 8: note = l-Phenylalanine-4-carboxylic acid
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
XXXXWXNFXSXFGX

SEQ ID NO: 133 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 5: note = (S)-2-Amino-3-(1H-pyrrolo[2,3-b]pyridin-3-yl)propan oic acid
  SITE 6: note = N-Methyl-d-glutamic acid
  SITE 8: note = (S)-2-Amino-3-(4-sulfamoylphenyl)propanoic acid
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
XXXXXENXXSXFGX

SEQ ID NO: 134 - skipped

SEQ ID NO: 135 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 6: note = (2R,4S)-4-Hydroxypyrrolidine-2-carboxylic acid
  SITE 8: note = (S)-2-Amino-3-(4-(carboxymethoxy)phenyl)propanoic acid
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-sulfamoylphenyl)propanoic acid
  SITE 12: note = (S)-2-Amino-3-(4-(carboxymethoxy)phenyl)propanoic acid
  SITE 13: note = Sarcosine
  SITE 14: note = (2S,3S)-2-Amino-3-(7-methoxy-1H-indol-3-yl)butanoic acid
XXXXWXNXXSXXGX

SEQ ID NO: 136 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 5: note = (S)-2-Amino-3-[1-(carboxymethyl)indol-3-yl]propanoi c acid
  SITE 6: note = 2-(((3S,5R)-5-Carboxypyrrolidin-3-yl)oxy)-N,N,N-tri methylethan-1-aminium
  SITE 8: note = l-Phenylalanine-4-carboxylic acid
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-sulfamoylphenyl)propanoic acid
  SITE 12: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
XXXXXXNFXSXXGX

SEQ ID NO: 137 - protein, 14 aa, synthetic construct
  SITE 1: note = (1R,3S)-3-aminocyclopentane-1-carboxylic acid
  SITE 2: note = l-Homoleucine
  SITE 3: note = 4-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 138 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = (S)-2-Amino-3-(1,3-thiazol-5-yl)propanoic acid
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 139 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 5: note = (S)-2-Amino-3-[1-(carboxymethyl)indol-3-yl]propanoi c acid
  SITE 6: note = 2-(((3S,5R)-5-Carboxypyrrolidin-3-yl)amino)-N,N, -trimethyl-2-oxoethan-1-aminium
  SITE 8: note = l-Phenylalanine-4-carboxylic acid
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
XXXXXXNFXSXFGX

SEQ ID NO: 140 - skipped

SEQ ID NO: 141 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = Sarcosine
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWGNXXXXYGX

SEQ ID NO: 142 - protein, 14 aa, synthetic construct
  SITE 1: note = (1R,3S)-3-aminocyclopentane-1-carboxylic acid
  SITE 2: note = O-Ethyl-l-serine
  SITE 3: note = 4-Pyridylalanine
  SITE 4: note = (2S,5R)-5-Hydroxypiperidine-2-carboxylic acid
  SITE 6: note = (2R,4S)-4-Hydroxypyrrolidine-2-carboxylic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XSXXWXNXXXXXGX

SEQ ID NO: 143 - protein, 14 aa, synthetic construct
  SITE 1: note = Acetyl Norleucine
  SITE 2: note = (2S,3R)-2-Amino-3-pyridin-4-ylbutanoic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXSXENXXXXYGXC

SEQ ID NO: 144 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 6: note = (2R,4S)-4-Aminopyrrolidine-2-carboxylic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWXNXXXXYGX

SEQ ID NO: 145 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 6: note = N-Methyl-l-glutamic acid
  SITE 8: note = (S)-2-amino-3-(6-(2-aminoethoxy)pyridin-3-yl)propan oic acid
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 146 - protein, 14 aa, synthetic construct
  SITE 1: note = Acetyl Ornithine
  SITE 3: note = l-2,3-Diaminopropionic acid
  SITE 4: note = (2S,3S)-2-Amino-3-(1H-indol-3-yl)butanoic acid
  SITE 5: note = d-Proline
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = alpha-Methyl-l-tryptophan
  SITE 14: note = Amidated residue
  CROSSLNK 1..14: note = Position 1 connected to position 14 via an Acetyl cross-linker
XYXXPNXXXXHGXC

SEQ ID NO: 147 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 5: note = (S)-2-Amino-3-(1H-pyrrolo[2,3-b]pyridin-3-yl)propan oic acid
  SITE 6: note = (3-Amino-3-oxopropyl)-l-alanine
  SITE 8: note = l-Phenylalanine-4-carboxylic acid
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
XXXXXXNFXSXXGX

SEQ ID NO: 148 - protein, 14 aa, synthetic construct
  SITE 1: note = (1R,3S)-3-Aminocyclopentane-1-carboxylic acid
  SITE 2: note = O-(2-Aminoethyl)-l-serine
  SITE 3: note = 4-Pyridylalanine
  SITE 4: note = (2S,5R)-5-Hydroxypiperidine-2-carboxylic acid
  SITE 5: note = (S)-2-Amino-3-(1H-pyrrolo[2,3-b]pyridin-3-yl)propan oic acid
  SITE 6: note = (2R,4S)-4-Hydroxypyrrolidine-2-carboxylic acid
  SITE 8: note = l-Phenylalanine-4-carboxylic acid
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
XXXXXXNFXSXFGX

SEQ ID NO: 149 - protein, 14 aa, synthetic construct
  SITE 1: note = (1R,3S)-3-Aminocyclopentane-1-carboxylic acid
  SITE 2: note = O-(Cyclopropylmethyl)-l-serine
  SITE 3: note = 4-Pyridylalanine
  SITE 4: note = (2S,5R)-5-Hydroxypiperidine-2-carboxylic acid
  SITE 5: note = (S)-2-Amino-3-(1H-pyrrolo[2,3-b]pyridin-3-yl)propan oic acid
  SITE 6: note = (2R,4S)-4-Hydroxypyrrolidine-2-carboxylic acid
  SITE 8: note = l-Phenylalanine-4-carboxylic acid
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
XXXXXXNFXSXFGX

SEQ ID NO: 150 - skipped

SEQ ID NO: 151 - protein, 14 aa, synthetic construct
  SITE 1: note = Acetyl Norleucine
  SITE 2: note = 4-Pyridylalanine
  SITE 3: note = (2S,5R)-5-Hydroxypiperidine-2-carboxylic acid
  SITE 4: note = 1-Methyl- l-tryptophan
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = (S)-2-Amino-3-(4-carbamoylphenyl)propanoic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXXENXXXXYGXC

SEQ ID NO: 152 - protein, 14 aa, synthetic construct
  SITE 1: note = Acetyl Norleucine
  SITE 2: note = (S)-2-amino-3-(3-hydroxy-1-bicyclo[1.1.1]pentanyl)p ropanoic acid
  SITE 4: note = (S)-2-amino-3-(1H-indazol-1-yl)propanoic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXTXENXXXXYGXC

SEQ ID NO: 153 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 12: note = (S)-2-Amino-3-(3-(2-amino-2-oxoethyl)phenyl)propano ic acid
  SITE 13: note = Sarcosine
  SITE 14: note = (2S,3S)-2-Amino-3-(7-methoxy-1H-indol-3-yl)butanoic acid
XXXXWENXXSFXGX

SEQ ID NO: 154 - skipped

SEQ ID NO: 155 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 6: note = (2R,4S)-4-Hydroxypyrrolidine-2-carboxylic acid
  SITE 8: note = 4-Pyridylalanine
  SITE 9: note = (S)-2-Amino-3-(7-cyano-1H-indol-3-yl)propanoic acid
  SITE 10: note = alpha-Methyl-l-serine
  SITE 11: note = (2S,3S)-2-Amino-3-(4-carbamoylphenyl)-3-hydroxyprop anoic acid
  SITE 12: note = 4-((1R,2S)-2-Amino-2-carboxy-1-hydroxyethyl)benzoic acid
  SITE 13: note = Sarcosine
  SITE 14: note = (2S,3S)-2-Amino-3-(7-methoxy-1H-indol-3-yl)butanoic acid
XXXXWXNXXSXXGX

SEQ ID NO: 156 - skipped

SEQ ID NO: 157 - protein, 14 aa, synthetic construct
  SITE 1: note = Acetyl Norleucine
  SITE 2: note = 4-Pyridylalanine
  SITE 3: note = (S)-Piperidine-2-carboxylic acid
  SITE 4: note = (S)-2-Amino-3-(1H-pyrrolo[2,3-b]pyridin-3-yl)propan oic acid
  SITE 5: note = 4-(Carboxymethylamino)butanoic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXXXNXXXXYGXC

SEQ ID NO: 158 - protein, 14 aa, synthetic construct
  SITE 1: note = Acetyl Norleucine
  SITE 2: note = 4-Pyridylalanine
  SITE 3: note = (S)-Piperazine-2-carboxylic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 11: note = (S)-2-Amino-3-[4-(trifluoromethoxy)phenyl]propanoic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXXENXXXXXGXC

SEQ ID NO: 159 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 5: note = (S)-2-Amino-3-(1H-pyrrolo[2,3-b]pyridin-3-yl)propan oic acid
  SITE 6: note = (2R,4S)-4-Hydroxypyrrolidine-2-carboxylic acid
  SITE 8: note = (S)-2-Amino-3-(pyrimidin-4-yl)propanoic acid
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
XXXXXXNXXSXFGX

SEQ ID NO: 160 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 12: note = (S)-2-amino-3-(6-(2-aminoethoxy)pyridin-3-yl)propan oic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXXGX

SEQ ID NO: 161 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = (2R,4S)-4-Aminopyrrolidine-2-carboxylic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWXNXXXXYGX

SEQ ID NO: 162 - protein, 14 aa, synthetic construct
  SITE 1: note = Acetyl Norleucine
  SITE 2: note = 4-Pyridylalanine
  SITE 3: note = (S)-Piperazine-2-carboxylic acid
  SITE 5: note = N-Methyl-l-glutamic acid
  SITE 7: note = (S)-2-Amino-3-(4-carbamoylphenyl)propanoic acid
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 11: note = (S)-2-Amino-3-(4-methoxyphenyl)propanoic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXWENXXXXXGXC

SEQ ID NO: 163 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 164 - protein, 14 aa, synthetic construct
  SITE 1: note = Acetyl Norleucine
  SITE 2: note = (S)-2-Amino-3-(4-carbamoylphenyl)propanoic acid
  SITE 3: note = (S)-2-amino-3-hydroxy-3-methylbutanoic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXXENXXXXYGXC

SEQ ID NO: 165 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 6: note = N-Methyl-l-glutamic acid
  SITE 8: note = (S)-2-Amino-3-(4-ureidophenyl)propanoic acid
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 166 - protein, 14 aa, synthetic construct
  SITE 1: note = (1R,3S)-3-aminocyclopentane-1-carboxylic acid
  SITE 2: note = N5-(pyridin-3-yl)-l-glutamine
  SITE 3: note = 4-Pyridylalanine
  SITE 4: note = N-Methyl-l-serine
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXSWENXXXXYGX

SEQ ID NO: 167 - protein, 14 aa, synthetic construct
  SITE 1: note = Acetyl Norleucine
  SITE 2: note = 4-Pyridylalanine
  SITE 3: note = (S)-Piperazine-2-carboxylic acid
  SITE 5: note = (R)-5-Azaspiro[2.4]heptane-6-carboxylic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXWXNXXXXYGXC

SEQ ID NO: 168 - protein, 14 aa, synthetic construct
  SITE 1: note = (S)-2-Ethylacetyl Norleucine
  SITE 2: note = 4-Pyridylalanine
  SITE 3: note = (2S,5R)-5-Hydroxypiperidine-2-carboxylic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = l-Phenylalanine-4-carboxylic acid
  SITE 8: note = (S)-2-Amino-3-(7-chloro-1H-indol-3-yl)propanoic acid
  SITE 9: note = alpha-Methyl-l-serine
  SITE 10: note = (S)-2-Amino-3-(4-carbamoylphenyl)propanoic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via a (S)-2-Ethylacetyl cross-linker
XXXXENXXXXYGXC

SEQ ID NO: 169 - skipped

SEQ ID NO: 170 - protein, 14 aa, synthetic construct
  SITE 1: note = Acetyl Norleucine
  SITE 2: note = 4-Pyridylalanine
  SITE 3: note = (S)-Piperidine-2-carboxylic acid
  SITE 4: note = (S)-2-Amino-3-(1H-pyrrolo[2,3-b]pyridin-3-yl)propan oic acid
  SITE 5: note = 2-(3-Hydroxypropylamino)acetic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXXXNXXXXYGXC

SEQ ID NO: 171 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = (S)-2-Amino-3-[4-(sulfomethyl)phenyl]propanoic acid
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 172 - protein, 14 aa, synthetic construct
  SITE 1: note = Acetyl Norleucine
  SITE 2: note = l-Homophenylalanine
  SITE 3: note = (S)-Piperazine-2-carboxylic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXXENXXXXYGXC

SEQ ID NO: 173 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = (S)-2-Amino-3-(4-sulfophenyl)propanoic acid
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 174 - skipped

SEQ ID NO: 175 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 12: note = (S)-2-Amino-3-(2-fluoro-4-hydroxyphenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXXGX

SEQ ID NO: 176 - skipped

SEQ ID NO: 177 - protein, 14 aa, synthetic construct
  SITE 1: note = Acetyl Norleucine
  SITE 2: note = (S)-2-Amino-3-(pyrimidin-5-yl)propanoic acid
  SITE 3: note = (S)-2-amino-3-hydroxy-3-methylbutanoic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXXENXXXXYGXC

SEQ ID NO: 178 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXHGX

SEQ ID NO: 179 - protein, 14 aa, synthetic construct
  SITE 1: note = Acetyl Norleucine
  SITE 2: note = 4-Pyridylalanine
  SITE 3: note = (S)-6,6-dimethylmorpholine-3-carboxylic acid
  SITE 5: note = N-Methyl-l-glutamic acid
  SITE 7: note = (S)-2-Amino-3-(4-(carboxymethoxy)phenyl)propanoic acid
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXWENXXXXYGXC

SEQ ID NO: 180 - protein, 14 aa, synthetic construct
  SITE 1: note = Acetyl Norleucine
  SITE 2: note = (2S,3S)-2-Amino-3-pyridin-4-ylbutanoic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXSXENXXXXYGXC

SEQ ID NO: 181 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 7: note = (S)-2-Amino-4-methoxybutanoic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWEXXXXXYGX

SEQ ID NO: 182 - protein, 14 aa, synthetic construct
  SITE 1: note = (1S,4R)-4-aminocyclopent-2-ene-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1S,4R)-4-aminocyclopent-2-ene-1-carboxylic acid
XXXXWENXXXXYGX

SEQ ID NO: 183 - protein, 14 aa, synthetic construct
  SITE 1: note = Acetyl Norleucine
  SITE 2: note = 4-Pyridylalanine
  SITE 3: note = (S)-6,6-dimethylmorpholine-3-carboxylic acid
  SITE 5: note = N-Ethyl-l-alanine
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXWANXXXXYGXC

SEQ ID NO: 184 - protein, 14 aa, synthetic construct
  SITE 1: note = Acetyl Norleucine
  SITE 2: note = 4-Pyridylalanine
  SITE 3: note = (S)-Piperazine-2-carboxylic acid
  SITE 5: note = N-Methyl-l-glutamic acid
  SITE 7: note = (S)-2-Amino-3-(4-carbamoylphenyl)propanoic acid
  SITE 8: note = (S)-2-Amino-3-(7-chloro-1H-indol-3-yl)propanoic acid
  SITE 9: note = alpha-Methyl-l-serine
  SITE 10: note = (S)-2-Amino-3-(4-carbamoylphenyl)propanoic acid
  SITE 11: note = (S)-3-(2-Amino-2-carboxyethyl)benzoic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXWENXXXXXGXC

SEQ ID NO: 185 - protein, 14 aa, synthetic construct
  SITE 1: note = Acetyl Norleucine
  SITE 2: note = 4-Pyridylalanine
  SITE 3: note = (2S,5R)-5-Hydroxypiperidine-2-carboxylic acid
  SITE 5: note = (2R,4S)-4-Hydroxypyrrolidine-2-carboxylic acid
  SITE 7: note = l-Phenylalanine-4-carboxylic acid
  SITE 8: note = (S)-2-Amino-3-(7-chloro-1H-indol-3-yl)propanoic acid
  SITE 9: note = alpha-Methyl-l-serine
  SITE 10: note = (S)-2-Amino-3-(4-carbamoylphenyl)propanoic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXWXNXXXXYGXC

SEQ ID NO: 186 - protein, 14 aa, synthetic construct
  SITE 1: note = Acetyl Norleucine
  SITE 2: note = 4-Pyridylalanine
  SITE 3: note = (2S,5R)-5-Hydroxypiperidine-2-carboxylic acid
  SITE 5: note = N-Ethyl-d-alanine
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = (S)-2-Amino-3-(4-carbamoylphenyl)propanoic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXWANXXXXYGXC

SEQ ID NO: 187-189 - skipped (3 entries)

SEQ ID NO: 190 - protein, 14 aa, synthetic construct
  SITE 1: note = (1R,3S)-3-aminocyclopentane-1-carboxylic acid
  SITE 2: note = (S)-2-amino-3-(1-bicyclo[1.1.1]pentanyl)propanoic acid
  SITE 3: note = 4-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 191 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 6: note = (2R,4S)-4-Hydroxypyrrolidine-2-carboxylic acid
  SITE 8: note = l-Phenylalanine-4-carboxylic acid
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = (S)-5-(2-Amino-2-carboxyethyl)pyrimidine-2-carboxyl ic acid
  SITE 13: note = Sarcosine
  SITE 14: note = (2S,3S)-2-Amino-3-(7-methoxy-1H-indol-3-yl)butanoic acid
XXXXWXNFXSXXGX

SEQ ID NO: 192 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = (2R,3S)-3-Hydroxypyrrolidine-2-carboxylic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWXNXXXXYGX

SEQ ID NO: 193-194 - skipped (2 entries)

SEQ ID NO: 195 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 12: note = (S)-2-Amino-3-[4-[2-(dimethylamino)ethoxy]phenyl]pr opanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXXGX

SEQ ID NO: 196 - skipped

SEQ ID NO: 197 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 5: note = (S)-2-Amino-3-[1-(carboxymethyl)indol-3-yl]propanoi c acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXXENXXXXYGX

SEQ ID NO: 198 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = (S)-2-amino-3-(3-fluoropyridin-4-yl)propanoic acid
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 199 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-sulfophenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 200 - skipped

SEQ ID NO: 201 - protein, 14 aa, synthetic construct
  SITE 1: note = (1R,3S)-3-aminocyclopentane-1-carboxylic acid
  SITE 2: note = l-Norleucine
  SITE 3: note = (S)-2-amino-3-(3-fluoropyridin-4-yl)propanoic acid
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 202 - skipped

SEQ ID NO: 203 - protein, 14 aa, synthetic construct
  SITE 1: note = Acetyl Norleucine
  SITE 2: note = 4-Pyridylalanine
  SITE 3: note = (2S,5R)-5-Hydroxypiperidine-2-carboxylic acid
  SITE 5: note = d-Proline
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXWPNXXXXYGXC

SEQ ID NO: 204 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 5: note = (S)-2-Amino-3-(2H-indazol-3-yl)propanoic acid
  SITE 6: note = (2R,4S)-4-(Carboxymethoxy)pyrrolidine-2-carboxylic acid
  SITE 8: note = l-Phenylalanine-4-carboxylic acid
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-sulfamoylphenyl)propanoic acid
  SITE 12: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
XXXXXXNFXSXFGX

SEQ ID NO: 205 - protein, 14 aa, synthetic construct
  SITE 1: note = Acetyl Norleucine
  SITE 2: note = (S)-2-Amino-3-(4-(aminomethyl)phenyl)propanoic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = (S)-2-Amino-3-(3-carbamoylphenyl)propanoic acid
  SITE 12: note = Sarcosine
  SITE 13: note = alpha-Methyl-l-tryptophan
  SITE 14: note = Amidated residue
  CROSSLNK 1..14: note = Position 1 connected to position 14 via an Acetyl cross-linker
XXSXENXXXXYGXC

SEQ ID NO: 206 - protein, 14 aa, synthetic construct
  SITE 1: note = Acetyl l-Citrulline
  SITE 2: note = (S)-2-Amino-3-(4-(aminomethyl)phenyl)propanoic acid
  SITE 4: note = (2S,3S)-2-Amino-3-(1H-indol-3-yl)butanoic acid
  SITE 5: note = d-Proline
  SITE 7: note = (S)-2-Amino-3-(4-(aminomethyl)phenyl)propanoic acid
  SITE 8: note = 3-(2-Naphthyl)-l-alanine
  SITE 9: note = alpha-Methyl-l-serine
  SITE 10: note = l-Phenylalanine-4-carboxylic acid
  SITE 11: note = (S)-2-Amino-3-(4-(aminomethyl)phenyl)propanoic acid
  SITE 12: note = Sarcosine
  SITE 13: note = (2S,3S)-2-Amino-3-(1H-indol-3-yl)butanoic acid
  SITE 14: note = Amidated residue
  CROSSLNK 1..14: note = Position 1 connected to position 14 via an Acetyl cross-linker
XXSXPNXXXXXGXC

SEQ ID NO: 207 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 6: note = (2R,4S)-4-Hydroxypyrrolidine-2-carboxylic acid
  SITE 8: note = (S)-4-(2-Amino-2-carboxyethyl)-2-chlorobenzoic acid
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = (S)-4-(2-Amino-2-carboxyethyl)-2-chlorobenzoic acid
  SITE 13: note = Sarcosine
  SITE 14: note = (2S,3S)-2-Amino-3-(7-methoxy-1H-indol-3-yl)butanoic acid
XXXXWXNXXSXXGX

SEQ ID NO: 208 - protein, 14 aa, synthetic construct
  SITE 1: note = Acetyl Norleucine
  SITE 2: note = 4-Pyridylalanine
  SITE 3: note = (S)-Piperidine-2-carboxylic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methyl-1H-indol-3-yl)butanoic acid
  SITE 14: note = Amidated residue
  CROSSLNK 1..14: note = Position 1 connected to position 14 via an Acetyl cross-linker
XXXWENXXXXYGXC

SEQ ID NO: 209 - protein, 14 aa, synthetic construct
  SITE 1: note = Acetyl Norleucine
  SITE 2: note = 4-Pyridylalanine
  SITE 3: note = N-Methyl-l-serine
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = l-Phenylalanine-4-carboxylic acid
  SITE 8: note = (S)-2-Amino-3-(7-chloro-1H-indol-3-yl)propanoic acid
  SITE 9: note = alpha-Methyl-l-serine
  SITE 10: note = (S)-2-Amino-3-(4-carbamoylphenyl)propanoic acid
  SITE 12: note = Sarcosine
  SITE 13: note = (2S,3S)-2-Amino-3-(4-methoxy-1H-indol-3-yl)butanoic acid
  SITE 14: note = Amidated residue
  CROSSLNK 1..14: note = Position 1 connected to position 14 via an Acetyl cross-linker
XXSXENXXXXYGXC

SEQ ID NO: 210 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 5: note = (S)-2-Amino-3-[1-(carboxymethyl)indol-3-yl]propanoi c acid
  SITE 6: note = (2R,4S)-4-Hydroxypyrrolidine-2-carboxylic acid
  SITE 8: note = l-Phenylalanine-4-carboxylic acid
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-sulfamoylphenyl)propanoic acid
  SITE 12: note = (2S,3R)-2-Amino-3-(4-carbamoylphenyl)-3-hydroxyprop anoic acid
  SITE 13: note = Sarcosine
  SITE 14: note = (2S,3S)-2-Amino-3-(7-methoxy-1H-indol-3-yl)butanoic acid
XXXXXXNFXSXXGX

SEQ ID NO: 211 - protein, 14 aa, synthetic construct
  SITE 1: note = Acetyl Norleucine
  SITE 2: note = 4-Pyridylalanine
  SITE 3: note = (2S,3R)-2-Amino-3-hydroxy-3-phenylpropanoic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXXENXXXXYGXC

SEQ ID NO: 212 - protein, 14 aa, synthetic construct
  SITE 1: note = Acetyl Norleucine
  SITE 2: note = 4-Pyridylalanine
  SITE 3: note = (S)-Piperidine-2-carboxylic acid
  SITE 5: note = N-Methyl-l-glutamic acid
  SITE 7: note = (S)-2-Amino-3-(4-(carboxymethyl)phenyl)propanoic acid
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXWENXXXXYGXC

SEQ ID NO: 213 - protein, 14 aa, synthetic construct
  SITE 1: note = (1R,3S)-3-aminocyclopentane-1-carboxylic acid
  SITE 2: note = (S)-2-amino-7,7,7-trifluoroheptanoic acid
  SITE 3: note = 4-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 214 - protein, 14 aa, synthetic construct
  SITE 1: note = (1S,3R)-3-aminocyclopentane-1-carboxylic acid
  SITE 2: note = l-Norleucine
  SITE 3: note = 4-Pyridylalanine
  SITE 4: note = (S)-6,6-dimethylmorpholine-3-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1S,3R)-3-aminocyclopentane-1-carboxylic acid
XXXXWENXXXXYGX

SEQ ID NO: 215 - protein, 14 aa, synthetic construct
  SITE 1: note = Acetyl l-Citrulline
  SITE 2: note = (S)-2-Amino-3-(4-(aminomethyl)phenyl)propanoic acid
  SITE 4: note = (2S,3S)-2-Amino-3-(1H-indol-3-yl)butanoic acid
  SITE 5: note = d-Proline
  SITE 7: note = l-Phenylalanine-4-carboxylic acid
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(1H-indol-3-yl)butanoic acid
  SITE 14: note = Amidated residue
  CROSSLNK 1..14: note = Position 1 connected to position 14 via an Acetyl cross-linker
XXSXPNXXXXHGXC

SEQ ID NO: 216 - protein, 14 aa, synthetic construct
  SITE 1: note = Acetyl Norleucine
  SITE 2: note = 4-Pyridylalanine
  SITE 3: note = (S)-Piperazine-2-carboxylic acid
  SITE 5: note = 2-[(3-Amino-3-oxopropyl)amino]acetic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXWXNXXXXYGXC

SEQ ID NO: 217 - protein, 14 aa, synthetic construct
  SITE 1: note = Acetyl Norleucine
  SITE 3: note = (S)-2-amino-3-hydroxy-3-methylbutanoic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XHXXENXXXXYGXC

SEQ ID NO: 218 - protein, 14 aa, synthetic construct
  SITE 1: note = Acetyl Norleucine
  SITE 2: note = 4-Pyridylalanine
  SITE 3: note = (S)-Piperazine-2-carboxylic acid
  SITE 5: note = N-Methyl-l-glutamic acid
  SITE 7: note = (S)-2-Amino-3-(4-methoxyphenyl)propanoic acid
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXWENXXXXYGXC

SEQ ID NO: 219 - protein, 14 aa, synthetic construct
  SITE 1: note = Acetyl Norleucine
  SITE 2: note = 4-Pyridylalanine
  SITE 3: note = (S)-Piperazine-2-carboxylic acid
  SITE 4: note = (S)-2-Amino-3-(1H-pyrrolo[2,3-b]pyridin-3-yl)propan oic acid
  SITE 5: note = (2R,4R)-4-Aminopyrrolidine-2-carboxylic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = (S)-2-Amino-3-(4-carbamoylphenyl)propanoic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXXXNXXXXYGXC

SEQ ID NO: 220 - protein, 14 aa, synthetic construct
  SITE 1: note = Acetyl Norleucine
  SITE 2: note = 4-Pyridylalanine
  SITE 3: note = (S)-Piperidine-2-carboxylic acid
  SITE 4: note = (S)-2-Amino-3-(1H-pyrrolo[2,3-b]pyridin-3-yl)propan oic acid
  SITE 5: note = 3-(Carboxymethylamino)propanoic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXXXNXXXXYGXC

SEQ ID NO: 221 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperidine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 12: note = (S)-2-Amino-3-(3-carbamoylphenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXXGX

SEQ ID NO: 222 - protein, 14 aa, synthetic construct
  SITE 1: note = Acetyl (S)-2-amino-3-(1-bicyclo[1.1.1]pentanyl)propanoic acid
  SITE 2: note = (S)-2-Amino-3-(3-(aminomethyl)phenyl)propanoic acid
  SITE 4: note = (S)-2-Amino-3-(7-chloro-1H-indol-3-yl)propanoic acid
  SITE 5: note = d-Proline
  SITE 6: note = N-Methyl-l-asparagine
  SITE 7: note = l-Phenylalanine-4-carboxylic acid
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 11: note = (S)-2-Amino-3-(3-(aminomethyl)phenyl)propanoic acid
  SITE 12: note = Sarcosine
  SITE 13: note = (S)-2-Amino-3-(4-chloro-1H-indol-3-yl)propanoic acid
  SITE 14: note = Amidated residue
  CROSSLNK 1..14: note = Position 1 connected to position 14 via an Acetyl cross-linker
XXSXPNXXXXXGXC

SEQ ID NO: 223 - protein, 14 aa, synthetic construct
  SITE 1: note = Acetyl Norleucine
  SITE 2: note = 4-Pyridylalanine
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = 1-Methyl- l-tryptophan
  SITE 14: note = Amidated residue
  CROSSLNK 1..14: note = Position 1 connected to position 14 via an Acetyl cross-linker
XXSXENXXXXYGXC

SEQ ID NO: 224 - protein, 14 aa, synthetic construct
  SITE 1: note = Acetyl Norleucine
  SITE 2: note = (S)-2-Amino-3-(4-(aminomethyl)phenyl)propanoic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = (S)-2-Amino-3-(4-(carboxymethyl)phenyl)propanoic acid
  SITE 12: note = Sarcosine
  SITE 13: note = alpha-Methyl-l-tryptophan
  SITE 14: note = Amidated residue
  CROSSLNK 1..14: note = Position 1 connected to position 14 via an Acetyl cross-linker
XXSXENXXXXYGXC

SEQ ID NO: 225 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 7: note = (S)-2-Amino-3-(carbamoylamino)propanoic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWEXXXXXYGX

SEQ ID NO: 226 - protein, 14 aa, synthetic construct
  SITE 1: note = Acetyl (S)-2-aminoheptanoic acid
  SITE 3: note = (S)-2-Amino-3-(piperazin-1-yl)propanoic acid
  SITE 4: note = (2S,3S)-2-Amino-3-(1H-indol-3-yl)butanoic acid
  SITE 5: note = d-Proline
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 11: note = 3-Pyridylalanine
  SITE 12: note = Sarcosine
  SITE 13: note = alpha-Methyl-l-tryptophan
  SITE 14: note = Amidated residue
  CROSSLNK 1..14: note = Position 1 connected to position 14 via an Acetyl cross-linker
XYXXPNXXXXXGXC

SEQ ID NO: 227 - protein, 14 aa, synthetic construct
  SITE 1: note = Acetylated residue
  SITE 2: note = 4-Pyridylalanine
  SITE 4: note = 3-(1-Naphthyl)-l-alanine
  SITE 5: note = N-Methyl-l-alanine
  SITE 7: note = 4-Pyridylalanine
  SITE 8: note = 3-(2-Naphthyl)-l-alanine
  SITE 9: note = alpha-Methyl-l-serine
  SITE 10: note = l-Phenylalanine-4-carboxylic acid
  SITE 12: note = Sarcosine
  SITE 14: note = Amidated residue
  CROSSLNK 1..14: note = Position 1 connected to position 14 via an Acetyl cross-linker
KXSXANXXXXYGWC

SEQ ID NO: 228 - protein, 14 aa, synthetic construct
  SITE 1: note = Acetyl Norleucine
  SITE 2: note = 4-Pyridylalanine
  SITE 3: note = (S)-Piperidine-2-carboxylic acid
  SITE 4: note = (S)-2-Amino-3-(1H-pyrrolo[2,3-b]pyridin-3-yl)propan oic acid
  SITE 5: note = 2-(2-Cyanoethylamino)acetic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXXXNXXXXYGXC

SEQ ID NO: 229 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXXGX

SEQ ID NO: 230 - protein, 14 aa, synthetic construct
  SITE 1: note = cis-4-aminocyclohexane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via cis-4-aminocyclohexane-1-carboxylic acid
XXXXWENXXXXYGX

SEQ ID NO: 231 - protein, 14 aa, synthetic construct
  SITE 1: note = Acetyl Norleucine
  SITE 2: note = 4-Pyridylalanine
  SITE 3: note = (S)-4-Amino-2-(methylamino)butanoic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = alpha-Methyl-l-tryptophan
  SITE 14: note = Amidated residue
  CROSSLNK 1..14: note = Position 1 connected to position 14 via an Acetyl cross-linker
XXXXENXXXXYGXC

SEQ ID NO: 232 - protein, 14 aa, synthetic construct
  SITE 1: note = Acetyl (S)-2-amino-3-(1-bicyclo[1.1.1]pentanyl)propanoic acid
  SITE 2: note = 4-Pyridylalanine
  SITE 4: note = (S)-2-Amino-3-(7-chloro-1H-indol-3-yl)propanoic acid
  SITE 5: note = N-Ethyl-l-alanine
  SITE 7: note = l-Phenylalanine-4-carboxylic acid
  SITE 8: note = (S)-2-Amino-3-(1H-pyrrolo[2,3-b]pyridin-3-yl)propan oic acid
  SITE 9: note = alpha-Methyl-l-serine
  SITE 10: note = l-Phenylalanine-4-carboxylic acid
  SITE 12: note = Sarcosine
  SITE 13: note = (S)-2-Amino-3-(1H-pyrrolo[2,3-b]pyridin-3-yl)propan oic acid
  SITE 14: note = Amidated residue
  CROSSLNK 1..14: note = Position 1 connected to position 14 via an Acetyl cross-linker
XXSXANXXXXYGXC

SEQ ID NO: 233 - protein, 14 aa, synthetic construct
  SITE 1: note = Acetyl L-Homophenylalanine
  SITE 2: note = 4-Pyridylalanine
  SITE 3: note = l-2,3-Diaminopropionic acid
  SITE 5: note = d-Proline
  SITE 7: note = 4-Pyridylalanine
  SITE 8: note = 3-(2-Naphthyl)-l-alanine
  SITE 9: note = alpha-Methyl-l-serine
  SITE 10: note = l-Phenylalanine-4-carboxylic acid
  SITE 11: note = 3-Pyridylalanine
  SITE 12: note = Sarcosine
  SITE 14: note = Amidated residue
  CROSSLNK 1..14: note = Position 1 connected to position 14 via an Acetyl cross-linker
XXXWPNXXXXXGWC

SEQ ID NO: 234 - protein, 14 aa, synthetic construct
  SITE 1: note = (1R,3S)-3-aminocyclopentane-1-carboxylic acid
  SITE 2: note = (S)-2-aminoheptanoic acid
  SITE 3: note = (2S,3S)-2-Amino-3-pyridin-4-ylbutanoic acid
  SITE 6: note = (2R,4S)-4-Hydroxypyrrolidine-2-carboxylic acid
  SITE 8: note = l-Phenylalanine-4-carboxylic acid
  SITE 9: note = (S)-2-amino-3-(7-cyanoindolyl)propionic acid
  SITE 10: note = alpha-Methyl-l-serine
  SITE 11: note = (S)-2-Amino-3-(4-carbamoylphenyl)propanoic acid
  SITE 12: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXTWXNXXXXXGX

SEQ ID NO: 235 - protein, 14 aa, synthetic construct
  SITE 1: note = Acetyl Norleucine
  SITE 2: note = 4-Pyridylalanine
  SITE 3: note = (S)-2-Amino-3-(methylamino)propanoic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = alpha-Methyl-l-tryptophan
  SITE 14: note = Amidated residue
  CROSSLNK 1..14: note = Position 1 connected to position 14 via an Acetyl cross-linker
XXXXENXXXXYGXC

SEQ ID NO: 236 - protein, 14 aa, synthetic construct
  SITE 1: note = Acetyl Norleucine
  SITE 2: note = 4-Pyridylalanine
  SITE 3: note = (S)-Piperazine-2-carboxylic acid
  SITE 5: note = d-Proline
  SITE 7: note = (S)-2-Amino-3-(furan-2-yl)propanoic acid
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXWPNXXXXYGXC

SEQ ID NO: 237 - protein, 14 aa, synthetic construct
  SITE 1: note = Acetyl (S)-2-aminoheptanoic acid
  SITE 2: note = 4-Pyridylalanine
  SITE 4: note = (2S,3S)-2-Amino-3-(1H-indol-3-yl)butanoic acid
  SITE 5: note = d-Proline
  SITE 7: note = l-Phenylalanine-4-carboxylic acid
  SITE 8: note = 3-(2-Naphthyl)-l-alanine
  SITE 9: note = alpha-Methyl-l-serine
  SITE 10: note = l-Phenylalanine-4-carboxylic acid
  SITE 12: note = Sarcosine
  SITE 13: note = alpha-Methyl-l-tryptophan
  SITE 14: note = Amidated residue
  CROSSLNK 1..14: note = Position 1 connected to position 14 via an Acetyl cross-linker
XXSXPNXXXXHGXC

SEQ ID NO: 238 - protein, 14 aa, synthetic construct
  SITE 1: note = (1R,3S)-3-aminocyclopentane-1-carboxylic acid
  SITE 2: note = (S)-2-Amino-6-(dimethylamino)hexanoic acid
  SITE 3: note = 4-Pyridylalanine
  SITE 4: note = (2S,5R)-5-Hydroxypiperidine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 239 - protein, 14 aa, synthetic construct
  SITE 1: note = Acetylated residue
  SITE 2: note = 4-Pyridylalanine
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (S)-2-Amino-3-(4-chloro-1H-indol-3-yl)propanoic acid
  SITE 14: note = Amidated residue
  CROSSLNK 1..14: note = Position 1 connected to position 14 via an Acetyl cross-linker
QXSXENXXXXYGXC

SEQ ID NO: 240 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = (S)-2-Amino-3-(4-carbamoylphenyl)propanoic acid
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 12: note = (S)-2-Amino-3-(4-methoxyphenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWEQXXXXXGX

SEQ ID NO: 241 - protein, 14 aa, synthetic construct
  SITE 1: note = (1R,3S)-3-aminocyclopentane-1-carboxylic acid
  SITE 2: note = l-Norleucine
  SITE 3: note = (S)-2-amino-3-(6-methoxypyridin-3-yl)propanoic acid
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 242 - protein, 14 aa, synthetic construct
  SITE 1: note = Acetylated residue
  SITE 5: note = d-Proline
  SITE 7: note = 4-Pyridylalanine
  SITE 9: note = alpha-Methyl-l-serine
  SITE 10: note = l-Phenylalanine-4-carboxylic acid
  SITE 12: note = Sarcosine
  SITE 14: note = Amidated residue
  CROSSLNK 1..14: note = Position 1 connected to position 14 via an Acetyl cross-linker
KYSWPQXWXXYGWC

SEQ ID NO: 243 - protein, 14 aa, synthetic construct
  SITE 1: note = Acetyl Norleucine
  SITE 2: note = (2S,3R)-2-Amino-3-(4-hydroxyphenyl)butanoic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXSXENXXXXYGXC

SEQ ID NO: 244 - protein, 14 aa, synthetic construct
  SITE 1: note = cis-4-aminocyclohexane-1-carboxylic acid
  SITE 2: note = l-Norleucine
  SITE 3: note = (S)-2-Amino-3-(4-(aminomethyl)phenyl)propanoic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via cis-4-aminocyclohexane-1-carboxylic acid
XXXSWENXXXXYGX

SEQ ID NO: 245 - protein, 14 aa, synthetic construct
  SITE 1: note = Acetyl (S)-2-Amino-6-azidohexanoic acid
  SITE 2: note = 4-Pyridylalanine
  SITE 3: note = l-2,3-Diaminopropionic acid
  SITE 4: note = (2S,3S)-2-Amino-3-(1H-indol-3-yl)butanoic acid
  SITE 5: note = d-Proline
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 11: note = (S)-2-Amino-3-(4-(aminomethyl)phenyl)propanoic acid
  SITE 12: note = Sarcosine
  SITE 13: note = alpha-Methyl-l-tryptophan
  SITE 14: note = Amidated residue
  CROSSLNK 1..14: note = Position 1 connected to position 14 via an Acetyl cross-linker
XXXXPNXXXXXGXC

SEQ ID NO: 246 - protein, 14 aa, synthetic construct
  SITE 1: note = (S)-2-Ethylacetyl Norleucine
  SITE 2: note = 4-Pyridylalanine
  SITE 3: note = (R)-thiomorpholine-3-carboxylic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = (S)-2-Amino-3-(4-carbamoylphenyl)propanoic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via a (S)-2-Ethylacetyl cross-linker
XXXWENXXXXYGXC

SEQ ID NO: 247 - skipped

SEQ ID NO: 248 - protein, 14 aa, synthetic construct
  SITE 1: note = (1R,4S)-4-aminocyclopent-2-ene-1-carboxylic acid
  SITE 2: note = l-Homophenylalanine
  SITE 3: note = 4-Pyridylalanine
  SITE 6: note = N-Methyl-l-alanine
  SITE 8: note = 4-Pyridylalanine
  SITE 10: note = alpha-Methyl-l-serine
  SITE 11: note = l-Phenylalanine-4-carboxylic acid
  SITE 13: note = Sarcosine
  SITE 1..14: note = Position 1 connected to position 14 via (1R,4S)-4-aminocyclopent-2-ene-1-carboxylic acid
XXXSWANXWXXYGW

SEQ ID NO: 249 - protein, 14 aa, synthetic construct
  SITE 1: note = Acetyl Norleucine
  SITE 2: note = 4-Pyridylalanine
  SITE 3: note = (S)-Piperazine-2-carboxylic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = (S)-3-(2-Amino-2-carboxyethyl)benzoic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXWENXXXXYGXC

SEQ ID NO: 250 - protein, 14 aa, synthetic construct
  SITE 1: note = (S)-2-Methylacetyl Norleucine
  SITE 2: note = (S)-2-Amino-3-(4-(aminomethyl)phenyl)propanoic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = (S)-2-Amino-3-(4-carbamoylphenyl)propanoic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via a (S)-2-Methylacetyl cross-linker
XXSXENXXXXYGXC

SEQ ID NO: 251 - protein, 14 aa, synthetic construct
  SITE 1: note = Acetyl Norleucine
  SITE 2: note = 4-Pyridylalanine
  SITE 3: note = (S)-Piperazine-2-carboxylic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 11: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXWENXXXXXGXC

SEQ ID NO: 252 - protein, 14 aa, synthetic construct
  SITE 1: note = Acetyl Norleucine
  SITE 2: note = (S)-2-Amino-3-(4-(aminomethyl)phenyl)propanoic acid
  SITE 5: note = N-Methyl-l-glutamic acid
  SITE 7: note = (2S,3S)-2-Amino-3-pyridin-4-ylbutanoic acid
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXSWENXXXXYGXC

SEQ ID NO: 253 - protein, 14 aa, synthetic construct
  SITE 1: note = Acetyl Norleucine
  SITE 2: note = 4-Pyridylalanine
  SITE 3: note = (S)-2-Amino-3-(1,3-thiazol-4-yl)propanoic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXXENXXXXYGXC

SEQ ID NO: 254 - skipped

SEQ ID NO: 255 - protein, 17 aa, synthetic construct
  SITE 1: note = Acetyl Norleucine
  SITE 2: note = 4-Pyridylalanine
  SITE 3: note = (S)-Piperazine-2-carboxylic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 17: note = Amidated residue
  CROSSLNK 1..14: note = Position 1 connected to position 14 via an Acetyl cross-linker
XXXXENXXXXYGXCGEE

SEQ ID NO: 256 - protein, 14 aa, synthetic construct
  SITE 1: note = Acetylated residue
  SITE 5: note = d-Proline
  SITE 6: note = l-Citrulline
  SITE 7: note = 4-Pyridylalanine
  SITE 9: note = alpha-Methyl-l-serine
  SITE 10: note = l-Phenylalanine-4-carboxylic acid
  SITE 12: note = Sarcosine
  SITE 14: note = Amidated residue
  CROSSLNK 1..14: note = Position 1 connected to position 14 via an Acetyl cross-linker
KYSWPXXWXXYGWC

SEQ ID NO: 257 - skipped

SEQ ID NO: 258 - protein, 14 aa, synthetic construct
  SITE 1: note = Acetyl Norleucine
  SITE 2: note = (S)-2-Amino-3-(4-(aminomethyl)phenyl)propanoic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Cysteamine
  CROSSLNK 1..14: note = Position 1 connected to position 14 via an Acetyl cross-linker
XXSXENXXXXYGXX

SEQ ID NO: 259 - protein, 14 aa, synthetic construct
  SITE 1: note = Acetyl Norleucine
  SITE 2: note = (2S,3R)-2-Amino-3-hydroxy-3-phenylpropanoic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXSXENXXXXYGXC

SEQ ID NO: 260 - protein, 14 aa, synthetic construct
  SITE 1: note = Acetyl L-Homophenylalanine
  SITE 5: note = N-Methyl-l-alanine
  SITE 7: note = 4-Pyridylalanine
  SITE 9: note = alpha-Methyl-l-serine
  SITE 10: note = l-Phenylalanine-4-carboxylic acid
  SITE 12: note = Sarcosine
  SITE 14: note = Amidated residue
  CROSSLNK 1..14: note = Position 1 connected to position 14 via an Acetyl cross-linker
XYSWANXWXXRGWC

SEQ ID NO: 261 - protein, 14 aa, synthetic construct
  SITE 1: note = Acetyl Norleucine
  SITE 2: note = 4-Pyridylalanine
  SITE 3: note = (S)-Piperazine-2-carboxylic acid
  SITE 4: note = (S)-2-Amino-3-(1H-pyrrolo[2,3-b]pyridin-3-yl)propan oic acid
  SITE 5: note = (2R,4R)-4-Hydroxypyrrolidine-2-carboxylic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXXXNXXXXYGXC

SEQ ID NO: 262 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 5: note = (S)-2-Amino-3-(7-chloro-1H-indol-3-yl)propanoic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXXENXXXXYGX

SEQ ID NO: 263 - protein, 14 aa, synthetic construct
  SITE 1: note = Acetyl L-Homophenylalanine
  SITE 4: note = (S)-2-Amino-3-(7-fluoro-1H-indol-3-yl)propanoic acid
  SITE 5: note = N-Methyl-l-alanine
  SITE 7: note = 4-Pyridylalanine
  SITE 9: note = alpha-Methyl-l-serine
  SITE 10: note = l-Phenylalanine-4-carboxylic acid
  SITE 12: note = Sarcosine
  SITE 14: note = Amidated residue
  CROSSLNK 1..14: note = Position 1 connected to position 14 via an Acetyl cross-linker
XYSXANXWXXYGWC

SEQ ID NO: 264 - protein, 14 aa, synthetic construct
  SITE 1: note = Acetyl Norleucine
  SITE 3: note = (S)-Piperazine-2-carboxylic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XHXXENXXXXYGXC

SEQ ID NO: 265 - protein, 14 aa, synthetic construct
  SITE 1: note = Acetyl L-Homophenylalanine
  SITE 4: note = (S)-2-amino-3-(1H-indazol-1-yl)propanoic acid
  SITE 5: note = N-Methyl-l-alanine
  SITE 7: note = 4-Pyridylalanine
  SITE 9: note = alpha-Methyl-l-serine
  SITE 10: note = l-Phenylalanine-4-carboxylic acid
  SITE 12: note = Sarcosine
  SITE 14: note = Amidated residue
  CROSSLNK 1..14: note = Position 1 connected to position 14 via an Acetyl cross-linker
XYSXANXWXXYGWC

SEQ ID NO: 266 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = (R)-4,4-Difluoropyrrolidine-2-carboxylic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWXNXXXXYGX

SEQ ID NO: 267 - protein, 14 aa, synthetic construct
  SITE 1: note = (1S,3R)-3-aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 6: note = N-Methyl-l-glutamic acid
  SITE 8: note = l-Phenylalanine-4-carboxylic acid
  SITE 9: note = (S)-2-Amino-3-(7-chloro-1H-indol-3-yl)propanoic acid
  SITE 10: note = alpha-Methyl-l-serine
  SITE 11: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1S,3R)-3-aminocyclopentane-1-carboxylic acid
XXXXWENXXXXYGX

SEQ ID NO: 268 - protein, 14 aa, synthetic construct
  SITE 1: note = cis-3-(aminomethyl)cyclobutane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via cis-3-(aminomethyl)cyclobutane-1-carboxylic acid
XXXXWENXXXXYGX

SEQ ID NO: 269 - protein, 14 aa, synthetic construct
  SITE 1: note = (1R,3S)-3-aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 12: note = (S)-2-Amino-3-(4-(aminomethyl)phenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXXGX

SEQ ID NO: 270 - protein, 14 aa, synthetic construct
  SITE 1: note = Acetyl Norleucine
  SITE 2: note = 4-Pyridylalanine
  SITE 3: note = N-Methyl-l-serine
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 11: note = (S)-2-Amino-3-(3-carbamoylphenyl)propanoic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXSWENXXXXXGXC

SEQ ID NO: 271 - protein, 14 aa, synthetic construct
  SITE 1: note = Acetyl Norleucine
  SITE 2: note = 4-Pyridylalanine
  SITE 3: note = (2S,4R)-4-Aminopiperidine-2-carboxylic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXWENXXXXYGXC

SEQ ID NO: 272 - protein, 14 aa, synthetic construct
  SITE 1: note = Acetyl Norleucine
  SITE 2: note = 4-Pyridylalanine
  SITE 3: note = (2S,5R)-5-Methylpiperidine-2-carboxylic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXWENXXXXYGXC

SEQ ID NO: 273 - protein, 14 aa, synthetic construct
  SITE 1: note = (S)-2-Methylacetyl Norleucine
  SITE 2: note = 4-Pyridylalanine
  SITE 3: note = (S)-2,3-diamino-3-methylbutanoic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = (S)-2-Amino-3-(4-carbamoylphenyl)propanoic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via a (S)-2-Methylacetyl cross-linker
XXXXENXXXXYGXC

SEQ ID NO: 274 - protein, 14 aa, synthetic construct
  SITE 1: note = (S)-2-Phenylacetyl l-Norleucine
  SITE 2: note = 4-Pyridylalanine
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = alpha-Methyl-l-tryptophan
  SITE 14: note = Amidated residue
  CROSSLNK 1..14: note = Position 1 connected to position 14 via a (S)-2-Phenylacetyl cross-linker
XXSXENXXXXYGXC

SEQ ID NO: 275 - protein, 14 aa, synthetic construct
  SITE 1: note = (1R,3S)-3-aminocyclopentane-1-carboxylic acid
  SITE 2: note = (S)-2-Amino-5,5-difluorohexanoic acid
  SITE 3: note = 4-Pyridylalanine
  SITE 4: note = (S)-Piperazine-2-carboxylic acid
  SITE 6: note = N-Methyl-l-glutamic acid
  SITE 8: note = 4-Pyridylalanine
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXXWENXXXXYGX

SEQ ID NO: 276 - protein, 14 aa, synthetic construct
  SITE 1: note = Acetyl Norleucine
  SITE 2: note = 4-Pyridylalanine
  SITE 3: note = (S)-Piperazine-2-carboxylic acid
  SITE 5: note = d-Proline
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = N-Methyl-d-alanine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXWPNXXXXYAXC

SEQ ID NO: 277 - protein, 14 aa, synthetic construct
  SITE 1: note = Acetyl Norleucine
  SITE 2: note = 4-Pyridylalanine
  SITE 3: note = l-2,4-diaminobutyric acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = alpha-Methyl-l-tryptophan
  SITE 14: note = Amidated residue
  CROSSLNK 1..14: note = Position 1 connected to position 14 via an Acetyl cross-linker
XXXXENXXXXYGXC

SEQ ID NO: 278 - protein, 14 aa, synthetic construct
  SITE 1: note = Acetylated residue
  SITE 2: note = 4-Pyridylalanine
  SITE 4: note = (S)-2-amino-3-(quinolin-4-yl)propanoic acid
  SITE 5: note = d-Proline
  SITE 7: note = 4-Pyridylalanine
  SITE 8: note = (S)-2-Amino-3-(1H-pyrrolo[2,3-b]pyridin-3-yl)propan oic acid
  SITE 9: note = alpha-Methyl-l-serine
  SITE 10: note = l-Phenylalanine-4-carboxylic acid
  SITE 13: note = (S)-2-Amino-3-(4-chloro-1H-indol-3-yl)propanoic acid
  SITE 14: note = Amidated residue
  CROSSLNK 1..14: note = Position 1 connected to position 14 via an Acetyl cross-linker
QXSXPNXXXXYPXC

SEQ ID NO: 279 - protein, 14 aa, synthetic construct
  SITE 1: note = Acetyl Norleucine
  SITE 2: note = 4-Pyridylalanine
  SITE 3: note = (S)-Piperazine-2-carboxylic acid
  SITE 5: note = (2R,4S)-4-Fluoropyrrolidine-2-carboxylic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = (2S,3S)-2-Amino-3-(7-methoxy-1H-indol-3-yl)butanoic acid
  SITE 14: note = Amidated residue
  CROSSLNK 1..14: note = Position 1 connected to position 14 via an Acetyl cross-linker
XXXWXNXXXXYGXC

SEQ ID NO: 280 - protein, 14 aa, synthetic construct
  SITE 1: note = (1R,3S)-3-aminocyclopentane-1-carboxylic acid
  SITE 2: note = (S)-2-aminoheptanoic acid
  SITE 3: note = (2S,3S)-2-Amino-3-pyridin-4-ylbutanoic acid
  SITE 6: note = (2R,4S)-4-Hydroxypyrrolidine-2-carboxylic acid
  SITE 8: note = l-Phenylalanine-4-carboxylic acid
  SITE 9: note = (S)-2-amino-3-(7-cyanoindolyl)propionic acid
  SITE 10: note = alpha-Methyl-l-serine
  SITE 11: note = (S)-2-Amino-3-(4-carbamoylphenyl)propanoic acid
  SITE 12: note = (S)-2-Amino-3-(4-carbamoylphenyl)propanoic acid
  SITE 13: note = Sarcosine
  SITE 14: note = (2S,3S)-2-Amino-3-(7-methoxy-1H-indol-3-yl)butanoic acid
  SITE 1..14: note = Position 1 connected to position 14 via (1R,3S)-3-aminocyclopentane-1-carboxylic acid
XXXEWXNXXXXXGX

SEQ ID NO: 281 - protein, 14 aa, synthetic construct
  SITE 1: note = Acetyl Norleucine
  SITE 2: note = 4-Pyridylalanine
  SITE 3: note = (S)-2-amino-2-(1-hydroxycyclopropyl)acetic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = l-Phenylalanine-4-carboxylic acid
  SITE 12: note = Sarcosine
  SITE 13: note = alpha-Methyl-l-tryptophan
  SITE 14: note = Amidated residue
  CROSSLNK 1..14: note = Position 1 connected to position 14 via an Acetyl cross-linker
XXXXENXXXXYGXC

SEQ ID NO: 282 - skipped

SEQ ID NO: 283 - protein, 14 aa, synthetic construct
  SITE 1: note = Acetyl Norleucine
  SITE 2: note = (S)-2-Amino-3-(4-(aminomethyl)phenyl)propanoic acid
  SITE 4: note = (S)-2-Amino-3-(1H-pyrrolo[2,3-b]pyridin-3-yl)propan oic acid
  SITE 5: note = N-Methyl-l-glutamic acid
  SITE 7: note = 4-Pyridylalanine
  SITE 8: note = (S)-2-Amino-3-(7-chloro-1H-indol-3-yl)propanoic acid
  SITE 9: note = alpha-Methyl-l-serine
  SITE 10: note = (S)-2-Amino-3-(4-(carboxymethoxy)phenyl)propanoic acid
  SITE 12: note = Sarcosine
  SITE 13: note = alpha-Methyl-l-tryptophan
  SITE 14: note = Amidated residue
  CROSSLNK 1..14: note = Position 1 connected to position 14 via an Acetyl cross-linker
XXSXENXXXXYGXC

SEQ ID NO: 284 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 5: note = (S)-2-Amino-3-(1H-pyrrolo[2,3-b]pyridin-3-yl)propan oic acid
  SITE 6: note = (2R,4S)-4-(Carboxymethoxy)pyrrolidine-2-carboxylic acid
  SITE 8: note = l-Phenylalanine-4-carboxylic acid
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
XXXXXXNFXSXFGX

SEQ ID NO: 285 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (2S,5R)-5-Hydroxypiperidine-2-carboxylic acid
  SITE 6: note = (2R,4S)-4-Hydroxypyrrolidine-2-carboxylic acid
  SITE 8: note = (S)-2-Amino-3-(4-(carboxymethoxy)phenyl)propanoic acid
  SITE 9: note = (S)-2-Amino-3-(7-cyano-1H-indol-3-yl)propanoic acid
  SITE 10: note = alpha-Methyl-l-serine
  SITE 11: note = (S)-2-Amino-3-(4-carbamoylphenyl)propanoic acid
  SITE 12: note = (S)-2-Amino-3-(4-(carboxymethoxy)phenyl)propanoic acid
  SITE 13: note = Sarcosine
  SITE 14: note = (2S,3S)-2-Amino-3-(7-methoxy-1H-indol-3-yl)butanoic acid
XXXXWXNXXSXXGX

SEQ ID NO: 286 - skipped

SEQ ID NO: 287 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 5: note = (S)-2-Amino-3-(1H-pyrrolo[2,3-b]pyridin-3-yl)propan oic acid
  SITE 6: note = (2R,4S)-4-(Dimethylamino)pyrrolidine-2-carboxylic acid
  SITE 8: note = l-Phenylalanine-4-carboxylic acid
  SITE 9: note = (S)-2-Amino-3-(7-cyano-1H-indol-3-yl)propanoic acid
  SITE 10: note = alpha-Methyl-l-serine
  SITE 11: note = l-Phenylalanine-4-carboxylic acid
  SITE 12: note = (2S,3R)-2-Amino-3-(4-carbamoylphenyl)-3-hydroxyprop anoic acid
  SITE 13: note = Sarcosine
  SITE 14: note = (2S,3S)-2-Amino-3-(7-methoxy-1H-indol-3-yl)butanoic acid
XXXXXXNFXSFXGX

SEQ ID NO: 288 - protein, 14 aa, synthetic construct
  SITE 1: note = (1R,3S)-3-Aminocyclopentane-1-carboxylic acid
  SITE 2: note = l-Norleucine
  SITE 3: note = 4-Pyridylalanine
  SITE 4: note = (S)-Piperazine-2-carboxylic acid
  SITE 6: note = N-Methyl-l-glutamic acid
  SITE 8: note = l-Phenylalanine-4-carboxylic acid
  SITE 9: note = (S)-2-Amino-3-(7-chloro-1H-indol-3-yl)propanoic acid
  SITE 10: note = alpha-Methyl-l-serine
  SITE 11: note = l-Phenylalanine-4-carboxylic acid
  SITE 13: note = Sarcosine
  SITE 14: note = (2S,3S)-2-Amino-3-(7-methoxy-1H-indol-3-yl)butanoic acid
XXXXWENFXSFYGX

SEQ ID NO: 289 - protein, 17 aa, synthetic construct
  SITE 5: note = Sarcosine
  SITE 12: note = Sarcosine
  SITE 17: note = Amidated Residue
  CROSSLNK 1..14: note = Position 1 connected to position 14 via an Acetyl cross-linker
FYSWGNYWSYYGWCGEE

SEQ ID NO: 290 - protein, 223 aa, unidentified
LVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTGGGLNDIFEAQKIEWHELEVLFQGPHHHHHHGSWSHPQFEK

SEQ ID NO: 291 - protein, 252 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTGGGLNDIFEAQKIEWHELEVLFQGPHHHHHHGSWSHPQFEK

SEQ ID NO: 292 - protein, 298 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDGGGLNDIFEAQKIEWHELEVLFQGPHHHHHHGSWSHPQFEK

SEQ ID NO: 293 - protein, 6 aa, synthetic construct
HHHHHH

SEQ ID NO: 294 - protein, 13 aa, synthetic construct
  SITE 1: note = 4-Pyridylalanine
  SITE 2: note = N-Methyl-l-serine t-butyl ester
  SITE 3: note = tert-Butoxy-carbonyl tryptophan
  SITE 4: note = N-Methyl-l-glutamic acid t-butyl ester
  SITE 5: note = Trityl-Asparagine
  SITE 6: note = 4-Pyridylalanine
  SITE 7: note = (S)-2-Amino-3-(7-chloro-1H-indol-3-yl)propanoic acid
  SITE 8: note = alpha-Methyl-l-serine t-butyl ester
  SITE 9: note = l-Phenylalanine-4-carboxylic acid t-butyl ester
  SITE 10: note = Tyrosine tert-Butyl
  SITE 11: note = Sarcosine
  SITE 12: note = (2S,3S)-2-Amino-3-(7-methoxy-1H-indol-3-yl)butanoic acid
  SITE 13: note = (1R,3S)-3-aminocyclopentane-1-carboxylic acid
  SITE 1..13: note = Position 1 connected to position 13 via an undefined amino acid compound
XSWENXXXXYGXX

SEQ ID NO: 295 - protein, 13 aa, synthetic construct
  SITE 1: note = 4-Pyridylalanine
  SITE 2: note = N-Methyl-l-serine t-butyl ester
  SITE 3: note = tert-Butoxy-carbonyl tryptophan
  SITE 4: note = N-Methyl-l-glutamic acid t-butyl ester
  SITE 5: note = Trityl-Asparagine
  SITE 6: note = 4-Pyridylalanine
  SITE 7: note = (S)-2-Amino-3-(7-chloro-1H-indol-3-yl)propanoic acid
  SITE 8: note = alpha-Methyl-l-serine t-butyl ester
  SITE 9: note = l-Phenylalanine-4-carboxylic acid t-butyl ester
  SITE 10: note = Tyrosine tert-Butyl
  SITE 11: note = Sarcosine
  SITE 12: note = (2S,3S)-2-Amino-3-(7-methoxy-1H-indol-3-yl)butanoic acid
  SITE 13: note = (1R,3S)-3-aminocyclopentane-1-carboxylic acid
  SITE 1..13: note = Position 1 connected to position 13 via an undefined amino acid compound
XSWENXXXXYGXX

SEQ ID NO: 296 - protein, 13 aa, synthetic construct
  SITE 1: note = 4-Pyridylalanine
  SITE 2: note = N-Methyl-l-serine t-butyl ester
  SITE 3: note = tert-Butoxy-carbonyl tryptophan
  SITE 4: note = N-Methyl-l-glutamic acid t-butyl ester
  SITE 5: note = Trityl-Asparagine
  SITE 6: note = 4-Pyridylalanine
  SITE 7: note = (S)-2-Amino-3-(7-chloro-1H-indol-3-yl)propanoic acid
  SITE 8: note = alpha-Methyl-l-serine t-butyl ester
  SITE 9: note = l-Phenylalanine-4-carboxylic acid t-butyl ester
  SITE 10: note = Tyrosine tert-Butyl
  SITE 11: note = Sarcosine
  SITE 12: note = (2S,3S)-2-Amino-3-(7-methoxy-1H-indol-3-yl)butanoic acid
  SITE 13: note = (1R,3S)-3-aminocyclopentane-1-carboxylic acid
  SITE 1..13: note = Position 1 connected to position 13 via an undefined amino acid compound
XSWENXXXXYGXX

SEQ ID NO: 297 - protein, 13 aa, synthetic construct
  SITE 1: note = 4-Pyridylalanine
  SITE 2: note = N-Methyl-l-serine
  SITE 4: note = N-Methyl-l-glutamic acid
  SITE 6: note = 4-Pyridylalanine
  SITE 7: note = (S)-2-Amino-3-(7-chloro-1H-indol-3-yl)propanoic acid
  SITE 8: note = alpha-Methyl-l-serine
  SITE 9: note = l-Phenylalanine-4-carboxylic acid
  SITE 11: note = Sarcosine
  SITE 12: note = (2S,3S)-2-Amino-3-(7-methoxy-1H-indol-3-yl)butanoic acid
  SITE 13: note = (1R,3S)-3-aminocyclopentane-1-carboxylic acid
  SITE 1..13: note = Position 1 connected to position 13 via an undefined amino acid compound
XSWENXXXXYGXX

SEQ ID NO: 298 - protein, 13 aa, synthetic construct
  SITE 1: note = 4-Pyridylalanine
  SITE 2: note = N-Methyl-l-serine t-butyl ester
  SITE 3: note = tert-Butoxy-carbonyl tryptophan
  SITE 4: note = N-Methyl-l-glutamic acid t-butyl ester
  SITE 5: note = Trityl-Asparagine
  SITE 6: note = 4-Pyridylalanine
  SITE 7: note = (S)-2-Amino-3-(7-chloro-1H-indol-3-yl)propanoic acid
  SITE 8: note = alpha-Methyl-l-serine t-butyl ester
  SITE 9: note = l-Phenylalanine-4-carboxylic acid t-butyl ester
  SITE 10: note = Tyrosine tert-Butyl
  SITE 11: note = Sarcosine
  SITE 12: note = (2S,3S)-2-Amino-3-(7-methoxy-1H-indol-3-yl)butanoic acid
  SITE 13: note = (1R,3S)-3-aminocyclopentane-1-carboxylic acid
  SITE 1..13: note = Position 1 connected to position 13 via an undefined amino acid compound
XSWENXXXXYGXX

SEQ ID NO: 299 - protein, 13 aa, synthetic construct
  SITE 1: note = 4-Pyridylalanine
  SITE 2: note = N-Methyl-l-serine t-butyl ester
  SITE 3: note = tert-Butoxy-carbonyl tryptophan
  SITE 4: note = N-Methyl-l-glutamic acid t-butyl ester
  SITE 5: note = Trityl-Asparagine
  SITE 6: note = 4-Pyridylalanine
  SITE 7: note = (S)-2-Amino-3-(7-chloro-1H-indol-3-yl)propanoic acid
  SITE 8: note = alpha-Methyl-l-serine t-butyl ester
  SITE 9: note = l-Phenylalanine-4-carboxylic acid t-butyl ester
  SITE 10: note = Tyrosine tert-Butyl
  SITE 11: note = Sarcosine
  SITE 12: note = (2S,3S)-2-Amino-3-(7-methoxy-1H-indol-3-yl)butanoic acid
  SITE 13: note = (1R,3S)-3-aminocyclopentane-1-carboxylic acid
  SITE 1..13: note = Position 1 connected to position 13 via an undefined amino acid compound
XSWENXXXXYGXX

SEQ ID NO: 300 - protein, 13 aa, synthetic construct
  SITE 1: note = 4-Pyridylalanine
  SITE 2: note = N-Methyl-l-serine t-butyl ester
  SITE 3: note = tert-Butoxy-carbonyl tryptophan
  SITE 4: note = N-Methyl-l-glutamic acid t-butyl ester
  SITE 5: note = Trityl-Asparagine
  SITE 6: note = 4-Pyridylalanine
  SITE 7: note = (S)-2-Amino-3-(7-chloro-1H-indol-3-yl)propanoic acid
  SITE 8: note = alpha-Methyl-l-serine t-butyl ester
  SITE 9: note = l-Phenylalanine-4-carboxylic acid t-butyl ester
  SITE 10: note = Tyrosine tert-Butyl
  SITE 11: note = Sarcosine
  SITE 12: note = (2S,3S)-2-Amino-3-(7-methoxy-1H-indol-3-yl)butanoic acid
  SITE 13: note = (1R,3S)-3-aminocyclopentane-1-carboxylic acid
  SITE 1..13: note = Position 1 connected to position 13 via an undefined amino acid compound
XSWENXXXXYGXX

SEQ ID NO: 301 - protein, 13 aa, synthetic construct
  SITE 1: note = 4-Pyridylalanine
  SITE 2: note = N-Methyl-l-serine
  SITE 4: note = N-Methyl-l-glutamic acid
  SITE 6: note = 4-Pyridylalanine
  SITE 7: note = (S)-2-Amino-3-(7-chloro-1H-indol-3-yl)propanoic acid
  SITE 8: note = alpha-Methyl-l-serine
  SITE 9: note = l-Phenylalanine-4-carboxylic acid
  SITE 11: note = Sarcosine
  SITE 12: note = (2S,3S)-2-Amino-3-(7-methoxy-1H-indol-3-yl)butanoic acid
  SITE 13: note = (1R,3S)-3-aminocyclopentane-1-carboxylic acid
  SITE 1..13: note = Position 1 connected to position 13 via an undefined amino acid compound
XSWENXXXXYGXX